(19) United States
(12) Patent Application Publication
(10) Pub. No.: US 2026/0265826 A1
(43) Pub. Date: Sep. 10, 2026

(54) COMPOSITIONS AND METHODS TO MODULATE TRANSPOSABLE ELEMENTS GOVERNING CELL-STATE TRANSITIONS

(71) Applicant: HAYA Therapeutics SA, Epalinges (CH)

(72) Inventors: Alexandra IOURANOVA, Chavannes-près-Renens (CH); Daniel Roman BLESSING, Saint-Sulpice (CH); Samir OUNZAIN, Lausanne (CH); Marco MINA, Epalinges (CH); René DREOS, Aran (CH); Diana Ivette CRUZ DÁVALOS, Ecublens (CH); Rudi MICHELETTI, San Diego, CA (US)

(21) Appl. No.: 19/394,421

(22) Filed: Nov. 19, 2025

Related U.S. Application Data
(63) Continuation of application No. PCT/IB2024/000260, filed on May 21, 2024.
(60) Provisional application No. 63/468,122, filed on May 22, 2023.
(60) Provisional application No. 63/536,192, filed on Sep. 1, 2023.

(51) Int. Cl. C12Q 1/6883; A61P 11/00; C12N 15/113

(52) U.S. Cl. CPC C12Q 1/6883; A61P 11/00; C12N 15/113; C12N 2310/11; C12N 2310/315; C12N 2310/321; C12N 2310/3231; C12N 2310/341; C12Q 2600/158

(57) ABSTRACT

Disclosed herein are modulators of a transposable element or a transposable element transcript, wherein the modulators induce or facilitate a change of a state of a cell from a first cellular state to a second cellular state. Further provided herein are synthetic polynucleic acids comprising a nucleic acid sequence of a transposable element or a portion thereof, wherein the synthetic polynucleic acids induce or facilitate a change of a state of a cell from a first cellular state to a second cellular state. Further provided herein are methods of preventing, alleviating, or treating pulmonary fibrosis in a subject in need thereof. Further provided herein are methods of cellular reprograming and reversing aging in a pulmonary tissue. Further provided herein are methods of diagnosing or prognosing pulmonary fibrosis.

### CROSS REFERENCE

This application is a continuation of International Application No. PCT/IB2024/000260 filed May 21, 2024, which claims the benefit of U.S. Provisional Application No. 63/468,122, filed May 22, 2023, and U.S. Provisional Application No. 63/536,192, filed Sep. 1, 2023, all of which are incorporated herein by reference in their entireties.

### SEQUENCE LISTING

The instant application contains a Sequence Listing which has been submitted electronically in XML format and is hereby incorporated by reference in its entirety. Said XML copy, created on Jun. 26, 2024, is named 60448-703-301_SL.xml and is 229,393 bytes in size.

### BACKGROUND OF THE DISCLOSURE

Transposable elements are DNA sequences that move from one location in the genome to another. Transposable element-derived sequences expressed alone or incorporated in other RNA transcripts are thought to play a role in gene regulatory networks. However, little has been dissected out regarding their role in governing cell-state transitions, which can be dynamically associated with disease progression in numerous clinical settings.

Therefore, there is a need for a better understanding of the transposable elements that drive cell-state transition and how to modulate the expression or activity of such transposable elements to prevent or treat the disease progression or symptoms related to the cell-state transitions.

### SUMMARY OF THE DISCLOSURE

The present disclosure provides the identification of exemplary transposable elements that play a role in cell-state transitions and disease progression and provides compositions and methods for treating, alleviating, or preventing diseases or symptoms thereof (e.g., lung fibrosis, heart fibrosis, or dermal fibrosis) by modulating (e.g., activating, suppressing, or supplementing) the expression or activity of the transposable elements. The present disclosure also teaches methods for reprogramming myofibroblasts or evaluating or monitoring efficacy of a treatment to induce pulmonary reprogramming. The present disclosure also teaches methods for methods of slowing or reversing aging of a pulmonary tissue or evaluating or monitoring pulmonary tissue regeneration. The present disclosure also teaches methods for methods of diagnosing or prognosing pulmonary fibrosis or for monitoring an efficacy of a pulmonary therapy.

Provided herein are modulators of a transposable element transcript, wherein the transposable element transcript is transcribed from a transposable element, wherein the modulator induces or facilitates a change of a state of a cell from a first cellular state to a second cellular state. In some instances, the first cellular state or the second cellular state comprises an extracellular-cellular-matrix synthesizing state, a cytokine-secreting state, an inflammatory state, a cell dividing state, a cell pausing state.

In some instances, the transposable element transcript modulates a functional RNA. In some instances, the functional RNA comprises a messenger RNA (mRNA), a long noncoding RNA (lncRNA), a circular RNA, a small nuclear RNAs (snRNAs), a small nucleolar RNAs (snoRNAs), a microRNA, an X-inactive specific transcript (xist) RNA, or a combination thereof.

In some instances, the transposable element or the transposable element transcript is associated with pulmonary fibrosis. In some instances, an RNA expression level of the transposable element transcript is increased in a cell affected by pulmonary fibrosis compared to a healthy cell.

In some instances, the transposable element belongs to ERV1, ERV2, or MaLR families. In some instances, the transposable element belongs to LTR16, LTR16A, LTR16A1, LTR16A2, LTR16B, LTR16B1, LTR16B2, LTR16C, LTR16D, LTR16D1, LTR16D2, LTR16E1, LTR16E2, ERV3-16A3_LTR, MER39, THE1A, THE1B, THE1C, THE1D, MSTA, MSTA1, MSTB, MSTB1, MSTB2, MSTC, MSTD, MLTIA0, MLTIA1, MLT1C, MLT1D, MLT1E, MLT1E1, MLT1E2, MLT1F, MLT1F1, MLT1F2, MLT1G1, MLT1G3, MLT1H, MLT1H1, MLT1I, MLT1J, MLT1J1, MLT1J2, MLT1K, MLT1L, MLT1M, MLT1N2, MLT1O, MLT2A1, MLT2A2, MLT2B1, MLT2B2, MLT2B3, MLT2B4, MLT2B5, MLT2C1, MLT2C2, MLT2D, MLT2E, MLT2F, MLT1A, MLT1B, MLT1E1A, MLT1E3, MLT1G, MLT1H2, MLT1C2, MLT1F-int, MST-int, MLT-int, MLT1H-int, MLT1J-int, THE1-int, HERV16, ERVL-E, HERVL, ERVL-B4, ERV3-16A3_I, LTR33, LTR50, LTR78, LTR41, LTR41B, HERV3-int, LTR13, HERVL18-int, MER66C, MER66D, or LTR19C subfamilies.

In some instances, the transposable element comprises a sequence that is at least 60%, at least 70%, at least 80%, at least 90%, or at least 95% identical to at least a fragment of a sequence selected from of SEQ ID NOs: 4, 10, 14, 15, 18, 27-32, and 34-38. In some instances, the transposable element comprises a sequence at least 60%, at least 70%, at least 80%, at least 90%, or at least 95% identical to at least a fragment of SEQ ID NO: 4. In some instances, the transposable element comprises a sequence of SEQ ID NO: 5, SEQ ID NO: 6, or wherein the transposable element or a fragment thereof is located in chr3: 45,818,736-45,818,762 (hg38) or chr3: 45,818,689-45,818,712 (hg38).

In some instances, the modulator comprises a nucleic-acid guided endonuclease complex, and wherein the nucleic acid targets the transposable element. In some instances, the modulator comprises a nucleic acid molecule that hybridizes to the transposable element, optionally wherein the nucleic acid molecule is a small interfering RNA (siRNA), a microRNA (miRNA), an inhibitory double stranded RNA (dsRNA), a small or short hairpin RNA (shRNA), an antisense oligonucleotide (ASO), a piwi-interacting RNA (piRNA), a heterogeneous nuclear RNA (hnRNA), a small nuclear RNA (snRNA), or an enzymatically-prepared siRNA (esiRNA) or the precursors thereof. In some instances, the nucleic acid molecule is an ASO.

In some instances, the ASO comprises at least 9 consecutive nucleotides with no more than 3 mismatches from SEQ ID NOs: 1-3. In some instances, the ASO comprises a nucleic acid sequence at least 80%, at least 85%, at least 90%, at least 95%, identical to a sequence selected from SEQ ID NOs: 1-3. In some instances, the ASO is a Gapmer with three consecutive LNAs in a 5′-wing region and two consecutive LNAs in a 3′-wing region. In some instances, one or more phosphodiester backbone is a phosphorothioate backbone.

Also provided herein are pharmaceutical compositions comprising the modulators described herein and a pharmaceutically acceptable salt, excipient, or derivative thereof. Also provided herein are kits comprising the modulators described herein or the pharmaceutical compositions.

Also provided herein are methods of preventing, alleviating, or treating pulmonary fibrosis in a subject in need thereof, the method comprising administering to the subject an effective amount of the modulators described herein or the pharmaceutical composition described herein. In some instances, the modulators described herein or the pharmaceutical composition described herein reduces expression of a fibrosis-related marker in a fibroblast affected by the pulmonary fibrosis. In some instances, the fibrosis-related marker comprises smooth muscle α actin (ACTA2), alpha 1 chain of collagen type I (COL1A1), alpha 1 chain of collagen type 3 (COL3A1), fibroblast activation protein (FAP), fibronectin 1 (FN1), periostin (POSTN), collagen triple helix repeat containing 1 (CTHRC1), alpha 1 chain of collagen type 10 (COL10A1), matrix metalloproteinase 11 (MMP11), thrombospondin 2 (THBS2), latent transforming growth factor beta binding protein 2 (LTBP2), Integrin-α11 (ITGA11), or a combination thereof. In some instances, the modulators described herein or the pharmaceutical composition described herein restore an expression of a set of genes that are aberrantly expressed in idiopathic pulmonary fibrosis (IPF) comparable to a healthy cell. In some instances, the set of genes comprises one or more genes identified in Table 3. In some instances, the modulators described herein or the pharmaceutical composition described herein release a cell from the subject from quiescence. In some instances, a percentage of fibrotic lesion surface is decreased, and such percentage of fibrotic lesion surface is measured by picrosirius red (PSR) staining.

Also provided herein are methods of reprogramming a myofibroblast into an early fibroblast precursor cell, a cell that is Top2a, Cenpa or ki67 positive, or a proliferating fibroblast, the method comprising contacting the myofibroblast with an effective amount of the modulators described herein or the pharmaceutical composition described herein. In some instances, the early fibroblast precursor cell is a mesothelial cell, an early fibroblast cell, a mid fibroblast cell, or a mesenchymal cell. In some instances, the cell that is Top2a, Cenpa or ki67 positive is a mesenchymal progenitor cell. In some instances, the mesenchymal progenitor cell is a bronchioalveolar stem cells (BASC, or called epithelial progenitor cell), an endothelial progenitor cell, or a fibroblast progenitor cell.

Also provided herein are methods of slowing or reversing aging of a pulmonary tissue in a subject, the method comprising administering to the subject an effective amount of the modulators described herein or the pharmaceutical composition described herein. In some instances, an upregulated proteostasis is observed in the pulmonary tissue. In some instances, an expression of Zmpste24 or Lamp2 is increased in the pulmonary tissue. In some instances, an upregulated macro-autophagy is observed in the pulmonary tissue. In some instances, the expression of Atg5 or Atg7 is increased in the pulmonary tissue. In some instances, a more active telomerase is observed in the pulmonary tissue. In some instances, an expression of Tert or Terc is increased in the pulmonary tissue. In some instances, an age-related inflammation or an age-related tissue repair capability is improved in the pulmonary tissue. In some instances, more M0 macrophages or fewer profibrotic M2 macrophages are observed in the pulmonary tissue. In some instances, In some instances, more M0 macrophages or fewer profibrotic M2 macrophages are observed in bronchoalveolar lavage (BAL) of the subject. In some instances, fewer white blood cells or fewer macrophages are observed in a bronchioalveolar lavage of the subject. In some instances, a number of total T cells or activated T cells is decreased in the pulmonary tissue. In some instances, a number of immunosuppressive Ctla4-expressing cells is increased in the pulmonary tissue. In some instances, a cell-cell communication between a fibroblast and an immune cell (e.g., a T cell, an M0, M1, or M2 macrophage) is decreased in the pulmonary tissue. In some instances, a cell-cell communication between a fibroblast and an immune cell (e.g., a T cell, an M0, M1, or M2 macrophage) is decreased in blood of the subject. In some instances, a cell-cell communication between a fibroblast and an immune cell (e.g., a T cell, an M0, M1, or M2 macrophage) is decreased in bronchoalveolar lavage (BAL) of the subject.

Also provided herein are methods for diagnosing or monitoring pulmonary fibrosis in a subject, the method comprising: (a) obtaining a biological sample derived from the subject; (b) detecting an amount and/or an activity of a plurality of biomarkers from the biological sample, wherein the plurality of biomarkers comprise a transposable element transcript transcribed from a transposable element, wherein the transposable element comprises a sequence that is at least 60%, at least 70%, at least 80%, at least 90%, or at least 95% identical to at least a portion of a sequence selected from of SEQ ID NOs: 4, 10, 14, 15, 18, 27-32, and 34-38; and (c) diagnosing the subject with pulmonary fibrosis or to have a high/higher chance to contract pulmonary fibrosis if the amount and/or the activity of the plurality of biomarkers is at least 50%, at least 60%, at least 70%, at least 80%, at least 90% higher when compared to a control. In some instances, the pulmonary fibrosis is idiopathic pulmonary fibrosis (IPF). In some instances, the sample is an alveolar type 2 cell (AT2 cell) or a lung fibroblast. In some instances, the methods further comprise d) administering to the subject the modulator described herein or the pharmaceutical composition described herein. In some instances, the modulator described herein or the pharmaceutical composition described herein is administered intratracheally, oropharyngeally, orally, nasally, intravenously, intraperitoneally, or intramuscularly. In some instances, the plurality of biomarkers comprise one or more fibrosis-related markers (e.g., smooth muscle α actin (ACTA2), alpha 1 chain of collagen type I (COL1A1), alpha 1 chain of collagen type 3 (COL3A1), fibroblast activation protein (FAP), fibronectin 1 (FN1), periostin (POSTN), collagen triple helix repeat containing 1 (CTHRC1), alpha 1 chain of collagen type 10 (COL10A1), matrix metalloproteinase 11 (MMP11), thrombospondin 2 (THBS2), latent transforming growth factor beta binding protein 2 (LTBP2), Integrin-α11 (ITGA11), or a combination thereof, or one or more genes identified in Table 3 and Table 5).

Also provided herein are methods of predicting severity and progression of pulmonary fibrosis in a subject, the method comprising: (a) obtaining a biological sample derived from the subject; (b) detecting an amount and/or an activity of a plurality of biomarkers from the biological sample, wherein the plurality of biomarkers comprise a transposable element transcript transcribed from a transposable element, wherein the transposable element comprises a sequence that is at least 60%, at least 70%, at least 80%, at least 90%, or at least 95% identical to at least a portion of a sequence selected from of SEQ ID NOs: 4, 10, 14, 15, 18, 27-32, and 34-38; and (c) predicting the subject to have a more severe or a progression of pulmonary fibrosis if the amount and/or the activity of the plurality of biomarkers is at least 50%, at least 60%, at least 70%, at least 80%, at least 90% higher when compared to a control. In some instances, the pulmonary fibrosis is idiopathic pulmonary fibrosis (IPF). In some instances, the sample is an alveolar type 2 cell (AT2 cell) or a lung fibroblast. In some instances, the methods further comprise d) administering to the subject the modulator described herein or the pharmaceutical composition described herein. In some instances, the modulator described herein or the pharmaceutical composition described herein is administered intratracheally, oropharyngeally, orally, nasally, intravenously, intraperitoneally, or intramuscularly. In some instances, the plurality of biomarkers comprise one or more fibrosis-related markers (e.g., smooth muscle α actin (ACTA2), alpha 1 chain of collagen type I (COL1A1), alpha 1 chain of collagen type 3 (COL3A1), fibroblast activation protein (FAP), fibronectin 1 (FN1), periostin (POSTN), collagen triple helix repeat containing 1 (CTHRC1), alpha 1 chain of collagen type 10 (COL10A1), matrix metalloproteinase 11 (MMP11), thrombospondin 2 (THBS2), latent transforming growth factor beta binding protein 2 (LTBP2), Integrin-α11 (ITGA11), or a combination thereof, or one or more genes identified in Table 3 and Table 5).

Also provided herein are methods for monitoring an efficacy of a pulmonary therapy in a subject, the method comprising: (a) obtaining a biological sample derived from the subject before and after the pulmonary therapy; (b) detecting an amount and/or an activity of a plurality of biomarkers from the biological sample, wherein the plurality of biomarkers comprise a transposable element transcript transcribed from a transposable element, wherein the transposable element comprises a sequence that is at least 60%, at least 70%, at least 80%, at least 90%, or at least 95% identical to at least a portion of a sequence selected from of SEQ ID NOs: 4, 10, 14, 15, 18, 27-32, and 34-38; and (c) concluding the pulmonary therapy is effective if the amount and/or the activity of the plurality of biomarkers after the pulmonary therapy is at least 50%, at least 60%, at least 70%, at least 80%, at least 90% lower when compared to the amount and/or the activity of the plurality of biomarkers before the pulmonary therapy. In some instances, the sample is an alveolar type 2 cell (AT2 cell) or a lung fibroblast. In some instances, if the pulmonary therapy is not effective as expected, the methods further comprise d) administering to the subject the modulator described herein or the pharmaceutical composition described herein. In some instances, the modulator described herein or the pharmaceutical composition described herein is administered intratracheally, oropharyngeally, orally, nasally, intravenously, intraperitoneally, or intramuscularly. In some instances, the plurality of biomarkers comprise one or more fibrosis-related markers (e.g., smooth muscle α actin (ACTA2), alpha 1 chain of collagen type I (COL1A1), alpha 1 chain of collagen type 3 (COL3A1), fibroblast activation protein (FAP), fibronectin 1 (FN1), periostin (POSTN), collagen triple helix repeat containing 1 (CTHRC1), alpha 1 chain of collagen type 10 (COL10A1), matrix metalloproteinase 11 (MMP11), thrombospondin 2 (THBS2), latent transforming growth factor beta binding protein 2 (LTBP2), Integrin-α11 (ITGA11), or a combination thereof, or one or more genes identified in Table 3 and Table 5).

Also provided herein are methods for evaluating or monitoring pulmonary tissue regeneration in a subject, the method comprising: (a) obtaining a biological sample derived from the subject at an earlier time point and a later time point; (b) detecting an amount and/or an activity of a plurality of biomarkers from the biological sample, wherein the plurality of biomarkers comprise a transposable element transcript transcribed from a transposable element, wherein the transposable element comprises a sequence that is at least 60%, at least 70%, at least 80%, at least 90%, or at least 95% identical to at least a portion of a sequence selected from of SEQ ID NOs: 4, 10, 14, 15, 18, 27-32, and 34-38; and (c) concluding with a positive pulmonary tissue regeneration if the amount and/or the activity of the plurality of biomarkers at the later time point is at least 50%, at least 60%, at least 70%, at least 80%, at least 90% lower when compared to the amount and/or the activity of the plurality of biomarkers at the earlier time point. In some instances, the sample is an alveolar type 2 cell (AT2 cell) or a lung fibroblast. In some instances, if the pulmonary tissue regeneration is not as expected (e.g., not enough regeneration), the methods further comprise d) administering to the subject the modulator described herein or the pharmaceutical composition described herein. In some instances, the modulator described herein or the pharmaceutical composition described herein is administered intratracheally, oropharyngeally, orally, nasally, intravenously, intraperitoneally, or intramuscularly. In some instances, the plurality of biomarkers comprise one or more fibrosis-related markers (e.g., smooth muscle α actin (ACTA2), alpha 1 chain of collagen type I (COL1A1), alpha 1 chain of collagen type 3 (COL3A1), fibroblast activation protein (FAP), fibronectin 1 (FN1), periostin (POSTN), collagen triple helix repeat containing 1 (CTHRC1), alpha 1 chain of collagen type 10 (COL10A1), matrix metalloproteinase 11 (MMP11), thrombospondin 2 (THBS2), latent transforming growth factor beta binding protein 2 (LTBP2), Integrin-α11 (ITGA11), or a combination thereof, or one or more genes identified in Table 3 and Table 5).

Also provided herein are methods for evaluating or monitoring efficacy of a treatment to induce pulmonary reprogramming in a subject, the method comprising: (a) obtaining a biological sample derived from the subject before and after the treatment; (b) detecting an amount and/or an activity of a plurality of biomarkers from the biological sample, wherein the plurality of biomarkers comprise a transposable element transcript transcribed from a transposable element, wherein the transposable element comprises a sequence that is at least 60%, at least 70%, at least 80%, at least 90%, or at least 95% identical to at least a portion of a sequence selected from of SEQ ID NOs: 4, 10, 14, 15, 18, 27-32, and 34-38; and (c) concluding the treatment is effective if the amount and/or the activity after the treatment is at least 50%, at least 60%, at least 70%, at least 80%, at least 90% lower when compared to the amount and/or the activity before the treatment. In some instances, the sample is an alveolar type 2 cell (AT2 cell) or a lung fibroblast. In some instances, if the pulmonary reprogramming is not sufficient, the methods further comprise d) administering to the subject the modulator described herein or the pharmaceutical composition described herein. In some instances, the modulator described herein or the pharmaceutical composition described herein is administered intratracheally, oropharyngeally, orally, nasally, intravenously, intraperitoneally, or intramuscularly. In some instances, the plurality of biomarkers comprise one or more fibrosis-related markers (e.g., smooth muscle α actin (ACTA2), alpha 1 chain of collagen type I (COL1A1), alpha 1 chain of collagen type 3 (COL3A1), fibroblast activation protein (FAP), fibronectin 1 (FN1), periostin (POSTN), collagen triple helix repeat containing 1 (CTHRC1), alpha 1 chain of collagen type 10 (COL10A1), matrix metalloproteinase 11 (MMP11), thrombospondin 2 (THBS2), latent transforming growth factor beta binding protein 2 (LTBP2), Integrin-α11 (ITGA11), or a combination thereof, or one or more genes identified in Table 3 and Table 5).

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The application contains at least one drawing executed in color. Copies of this patent or patent application publication with color drawing(s) will be provided by the Office upon request and payment of the necessary fee.

The novel features of the disclosure are set forth with particularity in the appended claims. A better understanding of the features and advantages of the present disclosure will be obtained by reference to the following detailed description that sets forth illustrative instances, in which the principles of the disclosure are utilized, and the accompanying drawings (also “Figure” and “FIG.” herein), of which:

FIGS. 1A-1M illustrate the transposable elements (TEs) that are associated with development and/or prognosis of pulmonary fibrosis. FIG. 1A shows that different TEs from different superfamilies/families/subfamilies exhibit different expression patterns in fibroblasts and myofibroblasts. FIG. 1B shows a metaplot of transcript factors binding. FIG. 1C shows leading TEs from the LTR/ERV family that have enriched binding with transcription factors EBF1 and SPI1. FIG. 1D shows LTR16C exhibits enriched binding with transcription factors EBF1 and SPI1. FIG. 1E shows the genomic position of a TE comprising a full length ERV3-16A3_I-int provirus flanked by two LTR16C terminal repeats. FIG. 1F shows the genomic position of a TE comprising LTR16E1. FIG. 1G shows reads from PRO-seq and deep RNA-seq data in and near the TE comprising two LTR16C and ERV3-16A3_I-int elements. FIG. 1H shows H3K27ac enrichment in LTR16C. FIG. 1I shows enrichment for EBF1 and SPI1 co-binding with different TEs from the LTR/ERV family. FIG. 1J shows expression of LTR16C in in vitro human lung fibroblasts cultured in serum-free medium containing 5 ng/ml TGFβ, measured via bulk RNA-sequencing. FIG. 1K shows EBF1 and SPI motifs in enhancer regions of various transposable elements. FIG. 1L shows transposable elements differentially expressed in myofibroblasts across different datasets. FIG. 1M shows filters that were excluded from the different datasets obtained to measure transposable element expression.

FIGS. 2A-2N illustrate the design of ASO-1, ASO-2, and ASO-3. FIG. 2A shows the hybridization/target regions of ASO-1, ASO-2, and ASO-3. FIG. 2B shows ACTA2 expression levels measured by qPCR in TGFβ-treated and serum starved fibroblasts transfected with an ASO (ASO-scrambled, ASO-1, or ASO-2) in comparison with control fibroblasts. FIG. 2C shows COL1A1 expression levels measured by qPCR in TGFβ-treated and serum starved fibroblasts with transfection of an ASO (ASO-scrambled, ASO-1, or ASO-2) in comparison with control fibroblasts. FIG. 2D shows COL3A1 expression levels measured by qPCR in TGFβ-treated and serum starved fibroblasts with transfection of an ASO (ASO-scrambled, ASO-1, or ASO-2) in comparison with control fibroblasts. FIG. 2E shows FAP expression levels measured by qPCR in TGFβ treated-fibroblasts with transfection of an ASO (ASO-scrambled, ASO-1, or ASO-2) in comparison with control fibroblasts. FIG. 2F shows FN1 expression levels measured by qPCR in TGFβ-treated and serum starved fibroblasts with transfection of an ASO (ASO-scrambled, ASO-1, or ASO-2) in comparison with control fibroblasts. FIG. 2G shows POSTN expression levels measured by qPCR in TGFβ-treated and serum starved fibroblasts with transfection of an ASO (ASO-scrambled, ASO-1, or ASO-2) in comparison with control fibroblasts. FIG. 2H shows ACTA2 expression levels measured by RNA-seq in TGFβ-treated and serum starved fibroblasts with transfection of an ASO (ASO-scrambled, ASO-1, or ASO-2) in comparison with control fibroblasts. FIG. 2I shows COL1A1 expression levels measured by RNA-seq in TGFβ-treated and serum starved fibroblasts with transfection of an ASO (ASO-scrambled, ASO-1, or ASO-2) in comparison with control fibroblasts. FIG. 2J shows COL3A1 expression levels measured by RNA-seq in TGFβ-treated and serum starved fibroblasts with transfection of an ASO (ASO-scrambled, ASO-1, or ASO-2) in comparison with control fibroblasts. FIG. 2K shows FAP expression levels measured by qPCR in TGFβ-treated and serum starved fibroblasts with transfection of an ASO (ASO-scrambled, ASO-1, or ASO-2) in comparison with control fibroblasts. FIG. 2L shows FN1 expression levels measured by RNA-seq in TGFβ-treated and serum starved fibroblasts with transfection of an ASO (ASO-scrambled, ASO-1, or ASO-2) in comparison with control fibroblasts. FIG. 2M shows POSTN expression levels measured by qPCR in TGFβ-treated and serum starved fibroblasts with transfection of an ASO (ASO-scrambled, ASO-1, or ASO-2) in comparison with control fibroblasts. FIG. 2N shows mismatch analysis of ASO-1 and ASO-3 to LTR16X subfamilies.

FIGS. 3A-3D illustrate the identification of gene signature of idiopathic pulmonary fibrosis (IPF) and evaluating the efficacy ASOs using the gene signature. FIG. 3A shows a workflow for identifying a group of genes that are differentially expressed in IPF. FIG. 3B shows the evaluation of ASOs using the IPF gene signature. FIG. 3C shows the evaluation of ASO-1 in human lung samples using the IPF gene signature. FIG. 3D shows gene ontology (GO) analysis of genes associated with IPF.

FIGS. 4A-4G illustrate the validation of ASO-3 that targets LTR16C. FIG. 4A shows the efficacy of ASO-3 in knocking down LTR16C. FIG. 4B shows ACTA2 expression levels measured by RNA-seq in TGFβ-treated and serum starved fibroblasts with transfection of an ASO (ASO-scrambled, ASO-1, or ASO-3) in comparison with control fibroblasts. FIG. 4C shows COL1A1 expression levels measured by RNA-seq in TGFβ-treated and serum starved fibroblasts with transfection of an ASO (ASO-scrambled, ASO-1, or ASO-3) in comparison with control fibroblasts. FIG. 4D shows COL3A1 expression levels measured by RNA-seq in TGFβ-treated and serum starved fibroblasts with transfection of an ASO (ASO-scrambled, ASO-1, or ASO-3) in comparison with control fibroblasts. FIG. 4E shows FAP expression levels measured by RNA-seq in TGFβ-treated and serum starved fibroblasts with transfection of an ASO (ASO-scrambled, ASO-1, or ASO-3) in comparison with control fibroblasts. FIG. 4F shows FN1 expression levels measured by RNA-seq in TGFβ-treated and serum starved fibroblasts with transfection of an ASO (ASO-scrambled, ASO-1, or ASO-3) in comparison with control fibroblasts. FIG. 4G shows POSTN expression levels measured by RNA-seq in TGFβ-treated and serum starved fibroblasts with transfection of an ASO (ASO-scrambled, ASO-1, or ASO-3) in comparison with control fibroblasts.

FIGS. 5A-5J illustrate dose-response analysis of ASO-1 and ASO-3 treatment. FIG. 5A shows qPCR results for COL3A1 across different doses of scrambled ASO, ASO-1, and ASO-3. FIG. 5B shows qPCR results for FN1 across different doses of scrambled ASO, ASO-1, and ASO-3. FIG. 5C shows qPCR results for POSTN across different doses of scrambled ASO, ASO-1, and ASO-3. FIG. 5D shows RNA-seq results for ACTA2 across different doses of scrambled ASO, ASO-1, and ASO-3. FIG. 5E shows RNA-seq results for COL1A1 across different doses of scrambled ASO, ASO-1, and ASO-3. FIG. 5F shows RNA-seq results for COL3A1 across different doses of scrambled ASO, ASO-1, and ASO-3. FIG. 5G shows RNA-seq results for FAP across different doses of scrambled ASO, ASO-1, and ASO-3. FIG. 5H shows RNA-seq results for FN1 across different doses of scrambled ASO, ASO-1, and ASO-3. FIG. 5I shows RNA-seq results for POSTN across different doses of scrambled ASO, ASO-1, and ASO-3. FIG. 5J shows RNA-seq results for the IPF gene signature across different doses of a scrambled ASO, ASO-1, and ASO-3.

FIGS. 6A-6V illustrate single nucleus RNA-sequencing (snRNA-seq) analysis upon ASO-1 and ASO-3 treatment. FIG. 6A shows Uniform Manifold Approximation and Projection (UMAP) analysis among fibroblasts, myofibroblasts, and myofibroblasts treated with scrambled ASO. FIG. 6B shows snRNA-seq results of COL3A1 expression in fibroblasts and myofibroblasts. FIG. 6C shows snRNA-seq results of COL1A1 expression in fibroblasts and myofibroblasts. FIG. 6D shows snRNA-seq results of ACTA2 expression in fibroblasts and myofibroblasts. FIG. 6E shows snRNA-seq results of POSTN expression in fibroblasts and myofibroblasts. FIG. 6F shows snRNA-seq results of FN1 expression in fibroblasts and myofibroblasts. FIG. 6G shows snRNA-seq results of FAP expression in fibroblasts and myofibroblasts. FIG. 6H shows cell cycle analysis of fibroblasts, myofibroblasts, and myofibroblasts treated with scrambled ASO. FIG. 6I shows UMAP analysis of myofibroblasts treated with a scrambled ASO, ASO-1, or ASO-3. FIG. 6J shows a violin plot of snRNA-seq results of COL3A1 expression in myofibroblasts treated with a scrambled ASO, ASO-1, or ASO-3. FIG. 6K shows a violin plot of snRNA-seq results of COL1A1 expression in myofibroblasts treated with a scrambled ASO, ASO-1, or ASO-3. FIG. 6L shows a violin plot of snRNA-seq results of POSTN expression in myofibroblasts treated with a scrambled ASO, ASO-1, or ASO-3. FIG. 6M shows a violin plot of snRNA-seq results of FN1 expression in myofibroblasts treated with a scrambled ASO, ASO-1, or ASO-3. FIG. 6N shows a violin plot of snRNA-seq results of FAP expression in myofibroblasts treated with a scrambled ASO, ASO-1, or ASO-3. FIG. 6O shows a heat map of snRNA-seq results of COL3A1 expression in myofibroblasts treated with a scrambled ASO, ASO-1, or ASO-3. FIG. 6P shows a heat map of snRNA-seq results of COL1A1 expression in myofibroblasts treated with a scrambled ASO, ASO-1, or ASO-3. FIG. 6Q shows a heat map of snRNA-seq results of FN1 expression in myofibroblasts treated with a scrambled ASO, ASO-1, or ASO-3. FIG. 6R shows a heat map of snRNA-seq results of FAP expression in myofibroblasts treated with a scrambled ASO, ASO-1, or ASO-3. FIG. 6S shows a heat map of snRNA-seq results of POSTN expression in myofibroblasts treated with a scrambled ASO, ASO-1, or ASO-3. FIG. 6T shows gene set enrichment analysis (GSEA) of the snRNA-seq data of the up-regulated pathways. FIG. 6U shows gene set enrichment analysis (GSEA) of the snRNA-seq data of the down-regulated pathways. FIG. 6V shows cell cycle analysis of myofibroblasts treated with a scrambled ASO, ASO-1, or ASO-3.

FIGS. 7A-7G illustrate anti-fibrotic effects of ASOs in aged mouse lung fibroblasts. FIG. 7A shows ACTA2 expression levels measured by RNA-seq in aged mouse lung fibroblasts. FIG. 7B shows COL1A1 expression levels measured by RNA-seq in aged mouse lung fibroblasts. FIG. 7C shows COL3A1 expression levels measured by RNA-seq in aged mouse lung fibroblasts. FIG. 7D shows FAP expression levels measured by RNA-seq in aged mouse lung fibroblasts. FIG. 7E shows FN1 expression levels measured by RNA-seq in aged mouse lung fibroblasts. FIG. 7F shows POSTN expression levels measured by RNA-seq in aged mouse lung fibroblasts. FIG. 7G shows Singscore IPF gene signature in aged mouse lung fibroblasts.

FIGS. 8A-8C illustrate an in vivo mouse model to test the effect of ASOs. FIG. 8A shows an experimental design of treating mice with ASOs. FIG. 8B shows body weight changes of the treated mice across 10 days. FIG. 8C shows body weight changes of the treated mice across 8 days.

FIGS. 9A-9I illustrate functional examination on lungs in the in vivo model. FIG. 9A shows bronchoalveolar lavage (BAL) counts in mice treated with vehicle, scrambled RNA, ASO-1, or ASO-3. FIG. 9B shows lung weights in mice treated with vehicle, scrambled RNA, ASO-1, or ASO-3. FIG. 9C shows lung to body weight ratio in mice treated with vehicle, scrambled RNA, ASO-1, or ASO-3. FIG. 9D shows enhanced pause in whole body plethysmography in mice treated with vehicle, scrambled RNA, ASO-1, or ASO-3. FIG. 9E shows lung resistance in mice treated with vehicle, scrambled RNA, ASO-1, or ASO-3. FIG. 9F shows lung elastance in mice treated with vehicle, scrambled RNA, ASO-1, or ASO-3. FIG. 9G shows lung compliance in mice treated with vehicle, scrambled RNA, ASO-1, or ASO-3. FIG. 9H shows liver weights in mice treated with vehicle, scrambled RNA, ASO-1, or ASO-3. FIG. 9I shows lung to body weight ratio in mice treated with vehicle, scrambled RNA, ASO-1, or ASO-3.

FIGS. 10A-11Z and 10AA-10CC illustrate various inflammatory markers in response to ASO treatment in the in vivo model. FIG. 10A shows IFNγ level in mice treated with vehicle, scrambled RNA, ASO-1, or ASO-3. FIG. 10B shows IL-1β level in mice treated with vehicle, scrambled RNA, ASO-1, or ASO-3. FIG. 10C shows IL-2 level in mice treated with vehicle, scrambled RNA, ASO-1, or ASO-3. FIG. 10D shows IL-4 level in mice treated with vehicle, scrambled RNA, ASO-1, or ASO-3. FIG. 10E shows IL-5 level in mice treated with vehicle, scrambled RNA, ASO-1, or ASO-3. FIG. 10F shows IL-6 level in mice treated with vehicle, scrambled RNA, ASO-1, or ASO-3. FIG. 10G shows KC/GRO (CXCL1) level in mice treated with vehicle, scrambled RNA, ASO-1, or ASO-3. FIG. 10H shows IL-10 level in mice treated with vehicle, scrambled RNA, ASO-1, or ASO-3. FIG. 10I shows IL-12p70 level in mice treated with vehicle, scrambled RNA, ASO-1, or ASO-3. FIG. 10J shows TNFα level in mice treated with vehicle, scrambled RNA, ASO-1, or ASO-3. FIG. 10K shows IL-9 level in mice treated with vehicle, scrambled RNA, ASO-1, or ASO-3. FIG. 10L shows MCP-1 level in mice treated with vehicle, scrambled RNA, ASO-1, or ASO-3. FIG. 10M shows IL-33 level in mice treated with vehicle, scrambled RNA, ASO-1, or ASO-3. FIG. 10N shows IL27p28/IL-30 level in mice treated with vehicle, scrambled RNA, ASO-1, or ASO-3. FIG. 10O shows IL-15 level in mice treated with vehicle, scrambled RNA, ASO-1, or ASO-3. FIG. 10P shows IL-17A/F level in mice treated with vehicle, scrambled RNA, ASO-1, or ASO-3. FIG. 10Q shows MIP-1a level in mice treated with vehicle, scrambled RNA, ASO-1, or ASO-3. FIG. 10R shows IP-10 level in mice treated with vehicle, scrambled RNA, ASO-1, or ASO-3. FIG. 10S shows MIP-2 level in mice treated with vehicle, scrambled RNA, ASO-1, or ASO-3. FIG. 10T shows plasma blood urea nitrogen level in mice treated with vehicle, scrambled RNA, ASO-1, or ASO-3. FIG. 10U shows plasma creatinine level in mice treated with vehicle, scrambled RNA, ASO-1, or ASO-3. FIG. 10V shows plasma phosphorus level in mice treated with vehicle, scrambled RNA, ASO-1, or ASO-3. FIG. 10W shows plasma calcium level in mice treated with vehicle, scrambled RNA, ASO-1, or ASO-3. FIG. 10X shows total plasma protein level in mice treated with vehicle, scrambled RNA, ASO-1, or ASO-3. FIG. 10Y shows plasma albumin level in mice treated with vehicle, scrambled RNA, ASO-1, or ASO-3. FIG. 10Z shows plasma globulin level in mice treated with vehicle, scrambled RNA, ASO-1, or ASO-3. FIG. 10AA shows plasma alanine transaminase level in mice treated with vehicle, scrambled RNA, ASO-1, or ASO-3. FIG. 10BB shows plasma aspartate transaminase level in mice treated with vehicle, scrambled RNA, ASO-1, or ASO-3. FIG. 10CC shows plasma alkaline phosphatase level in mice treated with vehicle, scrambled RNA, ASO-1, or ASO-3.

FIGS. 11A-11K illustrate anti-fibrotic effects of ASOs in the in vivo model. FIG. 11A shows down-regulated pathways in mice treated with ASO-1 compared to the ones treated with scrambled RNA. FIG. 11B shows gene ontology pathways in mice treated with ASO-1 compared to the ones treated with scrambled RNA. FIG. 11C shows up-regulated pathways in mice treated with ASO-1 compared to the ones treated with scrambled RNA. FIG. 11D shows down-regulated pathways in mice treated with ASO-3 compared to the ones treated with scrambled RNA. FIG. 11E shows up-regulated pathways in mice treated with ASO-1 compared to the ones treated with scrambled RNA. FIG. 11F shows Cend1 expression levels by RNA-seq in mice untreated (ctrl), or treated with ASO-scrambled (scr), ASO-1 or ASO-3. FIG. 11G shows Cdk1 expression levels by RNA-seq in mice untreated (ctrl), or treated with ASO-scrambled (scr), ASO-1 or ASO-3. FIG. 11H shows Clspn expression levels by RNA-seq in mice untreated (ctrl), or treated with ASO-scrambled (scr), ASO-1 or ASO-3. FIG. 11I shows Dt1 expression levels by RNA-seq in mice untreated (ctrl), or treated with ASO-scrambled (scr), ASO-1 or ASO-3. FIG. 11J shows Mad211 expression levels by RNA-seq in mice untreated (ctrl), or treated with ASO-scrambled (scr), ASO-1 or ASO-3. FIG. 11K shows Zwilch expression levels by RNA-seq in mice untreated (ctrl), or treated with ASO-scrambled (scr), ASO-1 or ASO-3.

FIG. 12 shows deeptools analysis illustrating H3K27ac+H3K4me1+ and H3K4me3+ transposable element groups in IPF in vitro model, indicating cluster 2 as an interesting target class.

FIGS. 13A-13B illustrates analysis of snATAC-seq of fibroblasts, myofibroblasts treated with scramble ASO, and myofibroblasts treated with ASO-1 or ASO-3. FIG. 13A shows example of LTR16C targeted by ASO-1 or ASO3 with loss of snATAC-seq peak. FIG. 13B shows example of LTR16A targeted by ASO-1 or ASO3 with loss of snATAC-seq peak.

FIG. 14 shows expression of IPF-associated genes profiled by bulk RNA-seq was decreased upon ASO treatment.

FIG. 15 shows FACS analysis of FAP protein expression on HLF treated with ASOs and differentiated to MyoFB.

FIGS. 16A-16D illustrate snRNA-seq analysis of primary HLF treated with ASOs and differentiated to MyoFB, integrated with human lung fetal atlas. FIG. 16A shows UMAP of snRNA-seq analysis of primary HLF treated with ASOs and differentiated to MyoFB, integrated with human lung fetal atlas. FIG. 16B shows UMAP of snRNA-seq analysis of primary HLF treated with ASOs and differentiated to MyoFB, integrated with human lung fetal atlas. FIG. 16C shows the different cell types that arise across the development progression. FIG. 16D shows an unsupervised pseudotime analysis.

FIG. 17 shows number of differentially open or differentially closed peaks obtained by snATAC-seq analysis of primary HLF treated with ASOs and differentiated to MyoFB. FIG. 17 also shows motif enrichment for transcription factors found in the corresponding open or closed peaks.

FIG. 18 shows cell type annotation (scType) on snRNA-seq performed on lung biopsies from Scramble- or LTR16 ASO-treated mice.

FIGS. 19A-19B illustrate expression of genes in mesenchymal progenitors. FIG. 19A shows expression heatmap of top 3 marker genes characteristic for each identified population, indicating that mesenchymal progenitors express Mki67, Top2a and Cenpa. FIG. 19B shows marker genes ranked by z-score, showing Mki67, Top2a and Cenpa to be highly expressed in mesenchymal progenitors.

FIGS. 20A-20B illustrate analysis of snRNA-seq data obtained from lung biopsies integrated with a mouse lung reference public dataset. FIG. 20A shows snRNA-seq performed on lung biopsies (n=2 animals/group) integrated with a mouse lung reference public dataset. Top section shows individual sample distribution on integrated UMAP. Bottom, left section shows cluster annotation: sample of origin, published cell type, cluster number. Bottom, right section shows cluster composition in percentages: sample of origin, published cell type. FIG. 20B shows UMAP generated with data obtained from snRNA-seq performed on lung biopsies integrated with a mouse lung reference public dataset.

FIGS. 21A-21D illustrate analysis of snRNA-seq data obtained from lung biopsies integrated published mouse mesenchymal cell type dataset. FIG. 21A shows UMAP analysis of selected annotated cell types indicating that the putative mesenchymal progenitor (Clusters 5, 7, 9) population appears transitional between fibroblast (Clusters 0, 3, 4) and AT2 (alveolar type II) (Clusters 1, 2, 6) population. Bottom portion shows increased expression of mouse orthologs of gene sets derived from human cell analysis in the putative mesenchymal progenitor population: LTR16 ASO-1 gene signature and early fibroblast gene signature. FIG. 21B shows UMAP analysis of selected annotated cell types indicating that the putative mesenchymal progenitor population appears transitional between fibroblast and AT2. FIG. 21C shows how each cluster of cell types were composed of myofibroblast treated with scramble, ASO-1, or ASO-3. FIG. 21D shows increased expression of mouse orthologs of gene sets derived from human cell analysis in the putative mesenchymal progenitor population: LTR16 ASO-1 gene signature and early fibroblast gene signature.

FIG. 22 shows the gene ontology analysis of gene identified for the bleomycin or mouse lung fibroblast signature.

FIG. 23 show high expression of bleomycin signature in bleomycin mice and mouse lung myofibroblasts.

FIG. 24A-24D illustrate snRNA-seq analysis of human lung fibroblasts treated with ASO-scramble control, ASO-1 or ASO-3. FIG. 24A shows UMAP of the snRNA-seq analysis.

FIG. 24B shows violin plots of snRNA-seq results for IPF gene signature, and expression of COL16A1, COL5A1, FAP, and FN1 among fibroblast or myofibroblasts treated with a scrambled ASO, ASOs. FIG. 24C shows UMAP of the snRNA-seq analysis, and gene ontology terms associated with genes downregulated by ASOs. FIG. 24D shows violin plots of snRNA-seq results for expression of COL1A1, COL3A1, FAP, and FN1 in each cell clusters.

FIG. 25 shows snRNA-seq based transposable element expression analysis on human lung myofibroblasts treated ASOs (ASO-1, ASO-3) or myofibroblasts with no treatment.

FIG. 26 shows UMAP of snATAC-seq analysis performed on fibroblasts, myofibroblasts, and myofibroblasts treated with scramble control (scr), ASO-1, or ASO-3.

FIG. 27 shows integration of snATAC-seq and snRNA-seq data, revealing distinct clusters representing fibroblasts, myofibroblasts, and myofibroblasts treated with ASOs.

FIG. 28 shows LTR16C expression in fibroblasts, ASO treated cells, and myofibroblasts.

FIG. 29 shows myofibroblast- and fibroblast-specific peak analysis of fibroblasts, myofibroblasts treated with scramble control, and myofibroblasts treated with ASO-1 or ASO-3.

FIGS. 30A-30B illustrates micro-C analysis of fibroblasts, myofibroblasts, and myofibroblast treated with scramble control, ASO-1 or ASO-3. FIG. 30A shows the percentage of how many 10 kb bins out of the total in the genome have transitioned from an HLF fibroblast to myofibroblast or HLF fibroblast to ASO-treated myofibroblast. FIG. 30B shows the percentage of how many 10 kb bins out of the total in the genome have transitioned from one cell state to another cell state.

FIG. 31 shows mean insulation score of fibroblast, myofibroblast, and myofibroblast treated scramble control, ASO-1, or ASO-3.

FIG. 32 shows snRNA-seq analysis of human lung fibroblasts, myofibroblasts, and myofibroblasts treated scramble control, ASO-1, or ASO-3 for expression of old signature and young signature.

FIGS. 33A-33C illustrate analysis of snRNA-seq data obtained from lung biopsies Scramble- or ASO-treated mice. FIG. 33A shows greater percentage of mesenchymal progenitors in lung biopsies of ASO-treated mice compared to scramble-treated mice. FIG. 33B shows quantification of mesenchymal progenitors in lung biopsies of ASO-treated mice compared to scramble-treated mice. FIG. 33C shows increased expression of a proliferating fibroblast gene signature in mesenchymal progenitors compared to other cell types.

FIGS. 34A-34C illustrate analysis of snRNA-seq data obtained from lung biopsies Scramble- or ASO-treated mice. FIG. 34A shows the activity of transcription factors, Ebfl and Sox2, in fibroblasts, mesenchymal progenitors, alveolar type II (AT2) upon ASO-1 or ASO-3 treatment. FIG. 34B shows the analysis of snRNA-seq data for the expression of old signature in different cell types. FIG. 34C shows the analysis of snRNA-seq data for the expression of young signature in different cell types.

FIG. 35 shows the analysis of analysis of snRNA-seq data for the expression of old and young signature in fibroblasts and AT2 upon scramble control, ASO-1 or ASO-3 treatment.

FIG. 36 shows expression of old and young signature in mouse lung upon scramble control, ASO-1 or ASO-3 treatment.

FIG. 37 shows aging rate of mice treated with scramble control, ASO-1 or ASO-3.

FIGS. 38A-38B illustrate expression of LTR16C in different cell types. FIG. 38A shows expression of LTR16C in smooth muscle cells, myofibroblasts, fibroblasts, PLIN2+ fibroblasts, mesothelial cells, and HAS1 high fibroblasts of publicly available idiopathic pulmonary fibrosis cell atlas. FIG. 38B shows expression of LTR16C in in vitro human lung fibroblasts cultured in serum-free medium containing 5 ng/ml TGFβ, measured via snRNA-seq. “DNA”=DNA transposons, “RC”=Rolling Circle, “LTR?”=LTRs annotated with some uncertainty, “DNA?”=DNA transposons with some uncertainty, “RC?”=Rolling Circle with some uncertainty.

FIGS. 39A-39B illustrate ChromHMM analysis of LTR16C elements in a lung fibroblast cell line (IMR90). FIG. 39A shows overlap of transposable elements and regulatory elements in a lung fibroblast cell line (IMR90). FIG. 39B shows LTR integrants overlapping with enhancers.

FIGS. 40A-40B illustrate snRNA-seq analysis of LTR16 coverage and chromatin state of myofibroblasts treated with scramble control, ASO-1, or ASO-3. FIG. 40A shows pseudobulk snATAC-seq analysis measuring LTR16 elements in ASO specific target space and all LTR16 elements in myofibroblasts treated with scramble control, ASO-1, or ASO-3. FIG. 40B shows snATAC-seq analysis measuring chromatin state in ASO specific target space and non-target ASO space in myofibroblasts treated with scramble control, ASO-1, or ASO-3.

FIG. 41 shows analysis of most response gene cluster in myofibroblast treated varying doses of ASO-1 or ASO-3.

FIG. 42 shows snRNA-seq analysis of LTR16C expression in bleomycin mouse model treated with scramble control compared to naive mice treated with vehicle control.

FIG. 43 shows experimental design of treating mice with vehicle, scramble control (non-LTR16 targeting oligonucleotide, 100 μg/mouse), ASO-1 (100 μg/mouse), or ASO-3 (100 μg/mouse).

FIGS. 44A-44F illustrate expression of Zmpste24, Atg5, and Tert in lung biopsies of mice treated with scramble control, ASO-1 or ASO-3, measured via snRNA-seq analysis. FIG. 44A shows UMAP of Zmpste24 expression in different cell types in lung biopsies of mice treated with scramble control, ASO-1 or ASO-3. FIG. 44B shows quantification of Zmpste24 expression in AT1, AT2, fibroblasts and mesenchymal progenitors in lung biopsies of mice treated with scramble control, ASO-1 or ASO-3. FIG. 44C shows UMAP of Atg5 expression in different cell types in lung biopsies of mice treated with scramble control, ASO-1 or ASO-3. FIG. 44D shows quantification of Atg5 expression in AT1, AT2, fibroblasts and mesenchymal progenitors in lung biopsies of mice treated with scramble control, ASO-1 or ASO-3. FIG. 44E shows UMAP of Tert expression in different cell types in lung biopsies of mice treated with scramble control, ASO-1 or ASO-3. FIG. 44F shows quantification of Tert expression in AT1, AT2, fibroblasts and mesenchymal progenitors in lung biopsies of mice treated with scramble control, ASO-1 or ASO-3.

FIG. 45 shows experimental design of treating naive mice with vehicle, scramble control (non-LTR16 targeting oligonucleotide, 4 mg/kg), ASO-1 (4 mg/kg), or ASO-3 (4 mg/kg) at day-1 and day 0 via oropharyngeal administration.

FIG. 46 shows Ki67 median score of ASO-treated mice compared to vehicle or scramble control treated mice at days 3, 5, and 7 of treatment.

FIGS. 47A-47F illustrate various readout of naive mice treated with vehicle, and bleomycin mouse model treated with scramble control, ASO-1, or ASO-3. FIG. 47A shows experimental design of treating naive mice with vehicle, and treating naive mice with bleomycin (1.35 mg/kg) at day 1, followed by treatment of scramble control (non-LTR16 targeting oligonucleotide, 4 mg/kg), ASO-1 (4 mg/kg), or ASO-3 (4 mg/kg) at day 11 and day 12 via oropharyngeal administration. FIG. 47B shows experimental design of treating naive mice with vehicle, and treating naive mice with bleomycin (1.35 mg/kg) at day 1, followed by treatment of scramble control (4 mg/kg), ASO-1 (4 mg/kg), or ASO-3 (4 mg/kg) at day 11 and day 12 via oropharyngeal administration. FIG. 47C shows probability of survival of mice treated with vehicle and bleomycin mouse model treated with scramble control, ASO-1, or ASO-3. FIG. 47D shows percentage of fibrosis in lungs of mice treated with vehicle and bleomycin mouse model treated with scramble control. FIG. 47E shows body weight (BW) change in mice treated with vehicle and bleomycin mouse model treated with scramble control over time. FIG. 47F shows percentage of fibrosis in lungs of mice treated with vehicle and bleomycin mouse model treated with scramble control, measured by quantifying picrosirius red (PSR) staining.

FIGS. 48A-48D illustrate analysis of snRNA-seq of lungs of bleomycin mouse model treated with ASOs. FIG. 48A shows emergence of three different subtypes of mesenchymal progenitors (bronchioalveolar stem cells (BASC), MESprog1, and MESprog2) in bleomycin mouse lungs treated with ASOs. BASC is also called epithelial progenitor cells. “MESprog1” is an internal label for endothelial progenitor cells. “MESprog2” is an internal label for fibroblast progenitor cells. FIG. 48B shows quantification of different cell types and all mesenchymal progenitors in lungs of mice treated with vehicle, and lungs of bleomycin mouse model treated with scramble control, ASO-1, or ASO-3. FIG. 48C shows quantification of BASC, endothelial progenitors, and fibroblast progenitors in lungs of mice treated with vehicle, and lungs of bleomycin mouse model treated with scramble control, ASO-1, or ASO-3. FIG. 48D shows composition of BASC, endothelial progenitors, and fibroblast progenitors in lungs of mice treated with vehicle, and lungs of bleomycin mouse model treated with scramble control, ASO-1, or ASO-3.

FIGS. 49A-49B illustrate quantification of macrophage populations in lungs of mice treated with vehicle, and lungs of bleomycin mouse model treated with scramble control, ASO-1, or ASO-3, measured via snRNA-seq analysis. FIG. 49A shows UMAP of macrophage clusters (M0, M1, M2 macrophages) of lungs of mice treated with vehicle, and lungs of bleomycin mouse model treated with scramble control, ASO-1, or ASO-3. FIG. 49B shows percentage of M0, M1, and M2 macrophages in lungs of treated with vehicle, and lungs of bleomycin mouse model treated with scramble control, ASO-1, or ASO-3.

FIGS. 50A-50B illustrate total white blood cells and macrophage numbers in bronchioalveolar lavage (BAL) of mice treated with vehicle, and bleomycin mouse model treated with scramble control, ASO-1 or ASO-3. FIG. 50A shows total white blood cells bronchioalveolar lavage (BAL) counts of mice treated with vehicle, and bleomycin mouse model treated with scramble control, ASO-1 or ASO-3. FIG. 50B shows macrophage numbers in bronchioalveolar lavage (BAL) of mice treated with vehicle, and bleomycin mouse model treated with scramble control, ASO-1 or ASO-3.

FIGS. 51A-51B illustrate absolute body weights and body weight changes over time of mice treated with vehicle, and bleomycin mouse model treated with scramble control, ASO-1 or ASO-3. FIG. 51A shows absolute body weights of mice treated with vehicle, and bleomycin mouse model treated with scramble control, ASO-1 or ASO-3. FIG. 51B shows body weight changes over time of mice treated with vehicle, and bleomycin mouse model treated with scramble control, ASO-1 or ASO-3.

FIG. 52 shows the quantification of total T cells, activated T cells, and immunosuppressive Ctla4-expressing cells in lungs of mice treated with vehicle, and lungs of bleomycin mouse model treated with scramble control, ASO-1, or ASO-3, measured via snRNA-seq analysis.

FIGS. 53A-53B illustrate analysis of cell-cell communication of mice treated with vehicle, and bleomycin mouse model treated with scramble control, ASO-1 or ASO-3. FIG. 53A shows fibroblast and immune cells (e.g., T cells, M0, M1, M2 macrophages) cell-cell communication in mice treated with bleomycin and scramble control compared to mice treated with vehicle or mice treated with bleomycin and ASO-3. FIG. 53B shows total number of ligand receptor pairs for fibroblast and immune cell communications in mice treated with vehicle and bleomycin mouse model treated with scramble control, ASO-1 or ASO-3.

FIGS. 54A-54F illustrate expression of Zmpste24, Atg5, and Tert in lung biopsies of mice treated with vehicle, and bleomycin mouse model treated with scramble control, ASO-1 or ASO-3, measured via snRNA-seq analysis. FIG. 54A shows UMAP of Tert expression in different cell types in lung biopsies of mice treated with scramble control, ASO-1 or ASO-3. FIG. 54B shows quantification of Tert expression in AT1, AT2, and fibroblasts in lung biopsies of mice treated with vehicle, and bleomycin mouse model treated with scramble control, ASO-1 or ASO-3. FIG. 54C shows UMAP of Atg5 expression in different cell types in lung biopsies of mice treated with scramble control, ASO-1 or ASO-3. FIG. 54D shows quantification of Atg5 expression in AT1, AT2, and fibroblasts in lung biopsies of mice treated with vehicle, and bleomycin mouse model treated with scramble control, ASO-1 or ASO-3. FIG. 54E shows UMAP of Zmpste24 expression in different cell types in lung biopsies of mice treated with scramble control, ASO-1 or ASO-3. FIG. 54F shows quantification of Zmpste24 expression in AT1, AT2, and fibroblasts in lung biopsies of mice treated with vehicle, and bleomycin mouse model treated with scramble control, ASO-1 or ASO-3.

FIGS. 55A-55D illustrate analysis of aging and fibrosis in lungs of bleomycin mouse model treated with scramble control, ASO-1 or ASO-3. FIG. 55A shows expression of young signature and old signature in bleomycin mouse model treated with scramble control, ASO-1 or ASO-3. FIG. 55B shows aging rate of bleomycin mouse model treated with scramble control, ASO-1 or ASO-3. FIG. 55C shows fibrosis area in lungs of bleomycin mouse model treated with scramble control, ASO-1 or ASO-3. FIG. 55D shows fibrosis severity in lungs of bleomycin mouse model treated with scramble control, ASO-1 or ASO-3.

FIG. 56 shows experimental design of treating naive mice with vehicle, and treating mice with bleomycin (1.35 mg/kg) at day 0, followed by treatment of scramble control (non-LTR16 targeting oligonucleotide, 4 mg/kg), ASO-1 (4 mg/kg), or ASO-3 (4 mg/kg) at days 11, 15, and 18 via oropharyngeal administration.

FIGS. 57A-57B illustrate gene expression signature and gene ontology of genes in aged mouse lung fibroblasts (MLF) treated with TGFβ and starvation, and with ASOs or scramble control. FIG. 57A shows Singscore bleomycin/mouse lung fibroblast (MLF) signature in aged fibroblast, aged myofibroblasts treated with scramble control, and aged myofibroblasts treated with ASO-3. FIG. 57B shows gene ontology analysis of genes upregulated in aged myofibroblasts treated with ASOs.

FIG. 58 shows experimental design of treating aged naive mice with vehicle, and treating aged naive mice with scramble control (non-LTR16 targeting oligonucleotide, 4 mg/kg), ASO-1 (4 mg/kg), or ASO-3 (4 mg/kg) at day-1 and day 0 via oropharyngeal administration. FIG. 58 also shows experimental design of treating aged mice with bleomycin (1.35 mg/kg) at day 0, followed by treatment of scramble control (non-LTR16 targeting oligonucleotide, 4 mg/kg), ASO-1 (4 mg/kg), or ASO-3 (4 mg/kg) at days 11, 15, and 18 via oropharyngeal administration.

FIG. 59 shows lung weights and lung weight to body weight ratio of mice treated with vehicle, and mice treated with bleomycin and scramble control, ASO-1, or ASO-3.

FIGS. 60A-60B illustrate functional lung capacity (enhanced pause (Penh)) of mice treated with vehicle, and mice treated with bleomycin and scramble control, ASO-1, or ASO-3, measured by performing whole body plethysmography (WBP). FIG. 60A shows Penh (dimensionless) of mice treated with vehicle, and mice treated with bleomycin and scramble control, ASO-1, or ASO-3. FIG. 60B shows Penh (area under curve) of mice treated with vehicle, and mice treated with bleomycin and scramble control, ASO-1, or ASO-3.

FIG. 61 shows tidal volume at day 14 and day 20 of mice treated with vehicle, and mice treated with bleomycin and scramble control, ASO-1, or ASO-3.

FIGS. 62A-62S illustrate measurement of various lung functions in mice treated with vehicle, and mice treated with bleomycin and scramble control, ASO-1, or ASO-3. FIG. 62A shows lung resistance of mice treated with vehicle, and mice treated with bleomycin and scramble control, ASO-1, or ASO-3. FIG. 62B shows lung compliance of mice treated with vehicle, and mice treated with bleomycin and scramble control, ASO-1, or ASO-3. FIG. 62C shows normalized functional residual capacity (FRC) of mice treated with vehicle, and mice treated with bleomycin and scramble control, ASO-1, or ASO-3. FIG. 62D shows normalized inspiratory capacity (IC) of mice treated with vehicle, and mice treated with bleomycin and scramble control, ASO-1, or ASO-3. FIG. 62E shows normalized vital capacity (VC) of mice treated with vehicle, and mice treated with bleomycin and scramble control, ASO-1, or ASO-3.

FIG. 62F shows normalized forced vital capacity (FVC) of mice treated with vehicle, and mice treated with bleomycin and scramble control, ASO-1, or ASO-3. FIG. 62G shows normalized forced expiratory volume over 100 ms (FEV100) of mice treated with vehicle, and mice treated with bleomycin and scramble control, ASO-1, or ASO-3. FIG. 62H shows FRC of mice treated with vehicle, and mice treated with bleomycin and scramble control, ASO-1, or ASO-3. FIG. 62I shows IC of mice treated with vehicle, and mice treated with bleomycin and scramble control, ASO-1, or ASO-3. FIG. 62J shows VC of mice treated with vehicle, and mice treated with bleomycin and scramble control, ASO-1, or ASO-3. FIG. 62K shows FVC of mice treated with vehicle, and mice treated with bleomycin and scramble control, ASO-1, or ASO-3. FIG. 62L shows FEV100 of mice treated with vehicle, and mice treated with bleomycin and scramble control, ASO-1, or ASO-3. FIG. 62M shows FEV100/FVC of mice treated with vehicle, and mice treated with bleomycin and scramble control, ASO-1, or ASO-3. FIG. 62N shows total lung capacity (TLC) of mice treated with vehicle, and mice treated with bleomycin and scramble control, ASO-1, or ASO-3. FIG. 62O shows Te of mice treated with vehicle, and mice treated with bleomycin and scramble control, ASO-1, or ASO-3. FIG. 62P shows Tocclude of mice treated with vehicle, and mice treated with bleomycin and scramble control, ASO-1, or ASO-3.

FIG. 62Q shows Cchord of mice treated with vehicle, and mice treated with bleomycin and scramble control, ASO-1, or ASO-3. FIG. 62R shows Cfvc50 of mice treated with vehicle, and mice treated with bleomycin and scramble control, ASO-1, or ASO-3. FIG. 62S shows residual volume (RV) of mice treated with vehicle, and mice treated with bleomycin and scramble control, ASO-1, or ASO-3.

FIG. 63 shows aspartate aminotransferase (AST), alanine transaminase (ALT), alkaline phosphatase (ALP) in plasma of mice treated with vehicle, and mice treated with bleomycin and scramble control, ASO-1, or ASO-3.

### DETAILED DESCRIPTION

The present disclosure includes that certain transposable elements (TEs) are associated with cell-state transition, and such TEs are key elements in regulating various elements in signaling pathways of cell-state transitions. As such, the present disclosure further includes that by modulating a transposable element (TE) associated with cell-state transition, the cell state of a diseased cell can be reverted to a cell state similar to a healthy counterpart.

Certain TEs play a role as gene regulatory elements, i.e., regulatory element for transcription. In some instances, certain TEs can regulate gene expression that are cell-, stage-, development-specific of an organism. Consistently, its expression and/or activity can be cell-, stage-, specific-, development-specific of an organism. The nature of TEs, having multiple copies of the same, also provides a golden opportunity for concerted regulation of the functional group by synchronizing all relevant transcriptional networks under the control of the transposable elements of the same subfamily.

However, one of the biggest challenges lies in the sheer volume of TEs, which takes up about 50% of the Human Genome. For example, using traditional methods, it is hardly possible to narrow down the TEs that exhibit precise, specific, and potent functions as cell-state reprogramming in fibrosis associated disease-driving cell states. In some instances, the classification and identification of the transposable element is based on Dfam, a source of consensus models and sequences. In some instances, the classification and identification of the transposable element is based on Repbase, a source of consensus models and sequences of transposable elements and other repeats. In some instances, the classification and identification of the transposable element is based on Repeatmasker, which includes a program that analyzes consensus models from outsides sources (e.g., Dfam or Repbase) and runs them on genomes for annotation, with a list of loci with coordinates as an output.

Described herein are modulators of a transposable element, wherein the modulators induce or facilitate a change of a state of a cell from a first cellular state to a second cellular state. Further described herein is a modulator comprising an inhibitory oligonucleotide (e.g., an antisense oligonucleotide (ASO)). In some instances, the ASO comprises at least 9 consecutive nucleotides with no more than 3 mismatches from 5′-CGGAGGCATGAGGTAG-3′ (SEQ ID NO: 1). Further described herein is a modulator comprising an antisense oligonucleotide (ASO), wherein the ASO comprises at least 9 consecutive nucleotides with no more than 3 mismatches from 5′-TGAGCAGGTTAGCACT-3′ (SEQ ID NO: 2). Further described herein in details is a modulator comprising an antisense oligonucleotide (ASO), wherein the ASO comprises at least 9 consecutive nucleotides with no more than 3 mismatches from 5′-CTACCTCATGCCTCCG-3′ (SEQ ID NO: 3). Further described herein are synthetic polynucleic acids comprising a nucleic acid sequence of a transposable element or a portion thereof, wherein the synthetic polynucleic acids induce or facilitate a change of a state of a cell from a first cellular state to a second cellular state. Further described herein are pharmaceutical compositions comprising the modulator described herein or the synthetic polynucleic acid described herein, and a pharmaceutically acceptable salt or derivative thereof. Further described herein are kits comprising the modulator described herein or the synthetic polynucleic acid described herein, or the pharmaceutical composition described herein. Further described herein are methods of preventing, alleviating, or treating pulmonary fibrosis in a subject in need thereof. Further described herein are methods of reprogramming a myofibroblast into a new cell type. Further described herein are methods of slowing or reversing aging of a pulmonary tissue in a subject. Further described herein are methods for diagnosing or monitoring pulmonary fibrosis in a subject. Further described herein are methods of predicting severity and progression of pulmonary fibrosis in a subject. Further described herein are methods of monitoring an efficacy of a pulmonary therapy in a subject. Further described herein are methods of evaluating or monitoring pulmonary tissue regeneration in a subject. Further described herein are methods of evaluating or monitoring efficacy of a treatment to induce pulmonary reprogramming.

### Regulatory Transposable Elements

Presence of certain TEs in the mammalian (e.g., human) genome are the results of ancient viral infections where a portion of the virus genome was integrated into the host (mammalian, human) genome. In some instances, the transposable element described herein is a class I transposable element, which comprises transposable elements that transpose through RNA intermediates. In some instances, the transposable element described herein is a class II transposable element, which comprises transposable elements that do not use RNA as a transposition intermediate.

There is a plurality of superfamilies of TEs (e.g., LINE and LTR), each of which comprises a plurality of families. For example, the superfamily of LINE comprises L1 (LINE-1) family, L2 (LINE-2) family, etc. LTR superfamily comprises LTR/ERV1, LTR-ERVL, LTR/ERVK families, etc. Each family further comprises a plurality of subfamilies. For example, L1 Hs and LIMd5 are two exemplary subfamilies of the family of L1 (LINE-1). L2c is an exemplary subfamily of the family of L2 (LINE-2). HERVH-int and LTR12 are two exemplary subfamilies of the family of LTR/ERV1. LTR16C is an exemplary subfamily of the family of LTR/ERVL. LTR5_Hs is an exemplary subfamily of the family of LTR/ERVK. See Carey et al., Mobile DNA volume 12, Article number: 4 (2021). In some instances, the leading candidates of transposable elements described herein are examined on the level of subfamily.

Expression of the certain transposable elements described herein may be tissue specific. In some instances, the transposable elements described herein are highly, exclusively, or distinctly expressed in one or more tissues or sub-tissues in the musculoskeletal system. In some instances, the transposable elements described herein are highly, exclusively, or distinctly expressed in one or more tissues in the digestive system (e.g., stomach, intestine, liver, pancreas, etc.) or one or more sub-tissues thereof. In some instances, the transposable elements described herein are highly, exclusively, or distinctly expressed in one or more tissues in the respiratory system (e.g., lungs) or one or more sub-tissues thereof. In some instances, the transposable elements described herein are highly, exclusively, or distinctly expressed in one or more tissues in the urinary system (e.g., kidney, bladder, etc.) or one or more sub-tissues thereof. In some instances, the transposable elements described herein are highly, exclusively, or distinctly expressed in one or more tissues in the reproductive system (e.g., ovaries, testes, etc.) or one or more sub-tissues thereof. In some instances, the transposable elements described herein are highly, exclusively, or distinctly expressed in one or more tissues in the endocrine system (e.g., pituitary gland, thyroid gland, etc.) or one or more sub-tissues thereof. In some instances, the transposable elements described herein are highly, exclusively, or distinctly expressed in one or more tissues in the circulatory system (e.g., hearts, arteries, veins, etc.) or one or more sub-tissues thereof. In some instances, the transposable elements described herein are highly, exclusively, or distinctly expressed in one or more tissues in the lymphatic system (e.g., lymph nodes, thymus, spleens, etc.) or one or more sub-tissues thereof. In some instances, the transposable elements described herein are highly, exclusively, or distinctly expressed in one or more tissues in the nervous system (e.g., brains, spinal cords, etc.) or one or more sub-tissues thereof. In some instances, the transposable elements described herein are highly, exclusively, or distinctly expressed in one or more tissues in the dermal system (e.g., skin, epithelium, etc.) or one or more sub-tissues thereof. As used herein, sub-tissues include one or more specific cell types of the tissue, or a fraction of the tissue. In some instances, sub-tissues may be distinguished from the rest of the tissue by its location, cell types, or functions.

In some instances, the transposable element modulates the regulatory networks associated with cell-state maintenance. In other instances, the transposable element modulates the regulatory networks associated with cell-state transition. In other instances, the transposable element induces a phenotypical change of a cell. In other instances, the transposable element modulates the regulatory networks associated with onset, development/progress, or prognosis of a fibrosis or symptoms thereof.

Accordingly, in some instances, a modulator modulating expression or function of the transposable element induces or facilitates a change of a state of a cell from a first cellular state to a second cellular state. In some instances, the modulator induces or facilitates a change of a state of a cell from a pathological cell state to a non-pathological cell state. In some instances, the pathological cell state is driven by environmental signals. In some instances, environmental signals include physiological stress, starvation, inflammatory cytokine signaling, differentiation signals, dedifferentiation signals, paracrine signaling, mechanical stress, or other signals thereof.

In some instances, the first cellular state or the second cellular state comprises an extracellular-cellular-matrix synthesizing state, a cytokine-secreting state, an inflammatory state, a cell dividing state, or a cell pausing state. In some instances, the first cellular state is the extracellular-cellular-matrix synthesizing state. In some instances, the second cellular state is the inflammatory state. Accordingly, in some instances, the modulators induce or facilitate a change of a state of a cell from the extracellular-cellular-matrix synthesizing state to the inflammatory state. In some instances, the modulators induce or facilitate a change of a state of a cell from the extracellular-cellular-matrix synthesizing state to the extracellular-cellular-matrix non-synthesizing state. In some instances, the modulators induce or facilitate a change of a state of a cell from the non-inflammatory state to the inflammatory state. In some instances, the modulators induce or facilitate a change of a state of a cell from the cell pausing state to the cell diving state.

In some instances, the first cellular state is a state that phenocopies a state of a fibroblast, an interstitial fibroblast, a resident fibroblast, a proliferative fibroblast, a EBF1+ fibroblast, a chondrocyte, a myofibroblast, a lipofibroblast, an alveolar fibroblast, a contractile myofibroblast, a quiescent myofibroblast, a pulmonary fibroblast progenitor, a cardiopulmonary fibroblast progenitor, a regeneration-competent fibroblast, an adventitial fibroblast, a peribronchial fibroblast, an airway fibroblast, an alveolar fibroblast, an early fibroblast, an early mesothelial cell, an intermediate fibroblast, a late mesothelial cell, a mesenchymal progenitor, a mesenchymal cell, a mid airway smooth muscle cell, a late airway smooth muscle cell, a mid airway fibroblast, or a pericyte. In some instances, the second cellular state is a state that phenocopies a state of a fibroblast, an interstitial fibroblast, a resident fibroblast, a proliferative fibroblast, a EBF1+ fibroblast, a chondrocyte, a myofibroblast, a lipofibroblast, an alveolar fibroblast, a contractile myofibroblast, a quiescent myofibroblast, a pulmonary fibroblast progenitor, a cardiopulmonary fibroblast progenitor, a regeneration-competent fibroblast, an adventitial fibroblast, a peribronchial fibroblast, an airway fibroblast, an alveolar fibroblast, an early fibroblast, an early mesothelial cell, an intermediate fibroblast, a late mesothelial cell, a mesenchymal progenitor, a mesenchymal cell, a mid airway smooth muscle cell, a late airway smooth muscle cell, a mid airway fibroblast, or a pericyte.

To maintain a cell state, to induce a cell-state transition, to induce a phenotypical change of a cell, to interfere with the onset of a fibrosis or symptoms thereof, to control the development/progress of a fibrosis or symptoms thereof, or to serve as a prognosis of a fibrosis or symptoms thereof, in some instances, the transposable element is differentially expressed in one cell state described herein. In some instances, the transposable element is differentially expressed in a fibrotic cell. In some instances, the transposable element is differentially expressed along with a differential expression of a marker gene associated with fibrosis.

To maintain a cell state, to induce a cell-state transition, to induce a phenotypical change of a cell, to interfere with the onset of a fibrosis or symptoms thereof, to control the development/progress of a fibrosis or symptoms thereof, or to serve as a prognosis of a fibrosis or symptoms thereof, in some instances, the transposable element modulates a functional RNA. In some instances, the functional RNA comprises a messenger RNA (mRNA), a long noncoding RNA (lncRNA), a circular RNA, a small nuclear RNAs (snRNAs), a small nucleolar RNAs (snoRNAs), a microRNA, an X-inactive specific transcript (xist) RNA, or a combination thereof. In some instances, the functional RNA comprises the lncRNA. Accordingly, in some instances, the transposable element modulates a lncRNA.

In some instances, the transposable element modulates expression or activity of a lncRNA. In some instances, the transposable element is transcribed from the same strand of the genome with the strand transcribing lncRNA modulated by the transposable element. In some instances, the transposable element is transcribed from a different strand (e.g., opposite strand) of the genome from the strand transcribing the lncRNA modulated by the transposable element.

To maintain a cell state, to induce a cell-state transition, to induce a phenotypical change of a cell, to interfere with the onset of a fibrosis or symptoms thereof, to control the development/progress of a fibrosis or symptoms thereof, or to serve as a prognosis of a fibrosis or symptoms thereof, in some instances, the transposable element is associated with certain enhancers and/or transcription factors. In some instances, the transposable element comprises an epigenetic modification or an epitranscriptomic modification. In some instances, the epigenetic modification comprises H3K27Ac, H3K4me3, H3K4me1, H4K16ac, H3K27me3, H3K79me2, H3K36me3, H2AFZ, H3K9ac, H3K4me2, H4K20me1, H2BK120ac, H3K56ac, H2AK9ac, H3K18ac, H4K5ac, H2AK5ac, H3K9me1, H3K4ac, H2BK5ac, H3K14ac, H3K79me1, H3K23ac, H2BK15ac, H3K4me2, H2BK12ac, H4K91ac, H4K20me1, H2BK20ac, H4K8ac, or a combination thereof. In some instances, an enhancer-binding transcriptional activator (e.g., bromodomain-containing protein 4 (BRD4)) binds to the epigenetic modification. In some instances, the epitranscriptomic modification comprises N6-methyladenine (m6A), N1-methyladenosine (m1A), inosine (I), pseudouridine (Ψ), 5-methylcytosine (m5C), methylguanosine (m1G), N6,N6-dimethyladenosine (m62A), 4-thiouridine (s4U), or a combination thereof. In some instances, the DNA sequence (e.g., genomic sequence) of the transposable element is bound by or bears a binding motif being bound by a transcription factor that is associated with cell-state maintenance, transition, phenotypical changes, onset, development/progress, or prognosis of a fibrosis or symptoms thereof.

To maintain a cell state, to induce a cell-state transition, to induce a phenotypical change of a cell, to interfere with the onset of a fibrosis or symptoms thereof, to control the development/progress of a fibrosis or symptoms thereof, or to serve as a prognosis of a fibrosis or symptoms thereof, in some instances, the transposable element directly binds to a target DNA, RNA, or protein to modulate the expression or activity of the target DNA, RNA, or protein. In other instances, the transposable element indirectly binds to a target DNA, RNA, or protein to modulate the expression or activity of the target DNA, RNA, or protein.

In some instances, the DNA copies of the transposable element described herein are identified by Nanopore direct RNA sequencing. In some instances, the DNA copies of the transposable element described herein are identified by total RNA-Seq. In some instances, the DNA copies of the transposable element described herein are identified by poly A-selected RNA-seq. In some instances, the DNA copies of the transposable element described herein are identified by GWAS and common variant calling. In some instances, the DNA copies of the transposable element described herein are identified by CUT and RUN probing, for example, histone modifications or transcription factors. In some instances, the DNA copies of the transposable element described herein are identified by ChIP-Seq probing, for example, histone modifications or transcription factors. In some instances, the DNA copies of the transposable element described herein are identified by Pro-Seq examining nascent transcription as a proxy for enhancer association. In some instances, the DNA copies of the transposable element described herein are identified by MicroC examining enhancer promoter contacts in 3D for gene regulation network. In some instances, the DNA copies of the transposable element described herein are identified by snATAC-Seq. In some instances, the DNA copies of the transposable element described herein are identified by bulk ATAC-seq. In some instances, the DNA copies of the transposable element described herein are identified by DNase-Seq.

In some instances, the transposable element described herein is a genetic component regulating any functional RNAs associated with an onset, development, or progress of a fibrosis as a master regulator. In some instances, the expression mapping of the transposable element described herein is assessed by total RNA-Seq. In some instances, the expression mapping of the transposable element described herein is assessed by Pro-Seq. In some instances, the expression mapping of the transposable element described herein is assessed by single-cell RNA (scRNA)-Seq. In some instances, the expression mapping of the transposable element described herein is assessed by single nuclear RNA (snRNA)-Seq.

In some instances, the RNA modifications and edits of the transposable element described herein is assessed by Nanopore direct RNA sequencing. In some instances, the RNA modifications and edits of the transposable element described herein is assessed by total RNA-Seq. In some instances, the RNA structure features of the transposable element described herein are assessed by Nanopore direct RNA sequencing. In some instances, the RNA structure features of the transposable element described herein are assessed by total RNA-Seq. In some instances, the RNA structure features of the transposable element described herein are assessed by poly A RNA-Seq. In some instances, the RNA structure features of the transposable element described herein are assessed by in silico predictive structural models.

In some instances, the binding of the transposable element described herein to one or more transcription factors and/or enhancers is assessed by CUT and RUN probing, for example, histone modifications or transcription factors. In some instances, the binding of the transposable element described herein to one or more transcription factors and/or enhancers is assessed by ChIP-Seq probing, for example, histone modifications or transcription factors. In some instances, the binding of the transposable element described herein to one or more transcription factors and/or enhancers is assessed by Pro-Seq examining nascent transcription as a proxy for enhancer association. In some instances, the binding of the transposable element described herein to one or more transcription factors and/or enhancers is assessed by MicroC examining enhancer promoter contacts in 3D for gene regulation network. In some instances, the binding of the transposable element described herein to one or more transcription factors and/or enhancers is assessed by snATAC-Seq. In some instances, the binding of the transposable element described herein to one or more RNA-binding proteins is assessed by enhanced crosslinking and immunoprecipitation followed by high-throughput sequencing (eCLIP), irCLIP, iCLIP, spyCLIP, HITS-CLIP, TLC-CLIP, or other variations thereof. See e.g., Van Nostrand et al., Nature Methods volume 13, pp. 508-514 (2016).

In some instances, the binding of the transposable element described herein to one or more RNA-binding protein is assessed by dataset mining focusing on RNA-binding protein such as T-cell intracellular antigen 1 related protein (TIAR).

In some instances, the enrichment of the transposable element described herein in one or more lncRNAs is assessed by investigating lncRNA catalogues developed from one or more databases. In some instances, the enrichment of the transposable element described herein in one or more lncRNAs is assessed by poly A RNASeq. In some instances, the enrichment of the transposable element described herein in one or more lncRNAs is assessed by total RNA-Seq. In some instances, the enrichment of the transposable element described herein in one or more lncRNAs is assessed by scRNA-Seq. In some instances, the enrichment of the transposable element described herein in one or more lncRNAs is assessed by snRNA-Seq.

Regulatory Transposable Elements Associated with Fibrosis

Provided herein are transposable elements identified by the methodologies described herein exhibit one or more features that are associated with fibrosis (e.g., DNA copies specifically expressed in fibrosis, RNA expression specifically associated with fibrosis, interaction with transcription factors and/or enhancers that are specifically associated with fibrosis, interaction with RNA-binding proteins that are specifically associated with fibrosis).

Idiopathic pulmonary fibrosis (IPF) is marked by uncontrolled proliferation and survival of activated fibroblasts, known as myofibroblasts, causing excessive extracellular matrix deposition, leading to lung fibrosis and damage to alveoli and bronchioles. Therefore, in some instances, targeting the lung fibrosis driving cell state, i.e. myofibroblasts, can significantly reduce IPF burden and promote lung rejuvenation.

In some instances, the transposable element is differentially expressed in a cell where a fibrosis is induced. In some instances, the transposable element is differentially expressed in a cell where a fibrosis progresses. In some instances, the transposable element is differentially expressed in a tissue where a fibrosis is induced. In some instances, the transposable element is differentially expressed in a tissue where a fibrosis progresses. In some instances, the differential expression of the transposable element is associated with the differential expression of a RNA, a protein or an element of a signaling pathway related to the initiation, development, progress, or prognosis of a fibrosis. In some instances, the transposable element regulates the differential expression of a RNA, a protein or an element of a signaling pathway related to the initiation, development, progress, or prognosis of a fibrosis. Therefore, in some instances, modulating the transposable element induces a cell-state transition in a cell affected by fibrosis. In some instances, modulating the transposable element induces a cell-state transition in a cell expected to be affected by fibrosis. In some instances, modulating the transposable element prevents the initiation and/or progression of fibrosis or its symptoms thereof. In some instances, modulating the transposable element reverses the progression of fibrosis. In some instances, modulating the transposable element alleviates or cure the symptoms of the fibrosis.

In some instances, the transposable element is associated with pulmonary fibrosis. In some instances, the pulmonary fibrosis is idiopathic pulmonary fibrosis. In some instances, an RNA expression level of the transposable element is altered in a cell affected by pulmonary fibrosis compared to a healthy cell. In some instances, the RNA expression level of the transposable element is increased in a cell affected by pulmonary fibrosis compared to a healthy cell. In some instances, the RNA expression level of the transposable element is at least 10%, 20%, 30%, 40%, 50%, 60%, 70, 80%, or 90% more than in a cell affected by pulmonary fibrosis compared to a healthy cell. In some instances, the RNA expression level of the transposable element is at least 1.5 folds, 2 folds, 3 folds, 4 folds, 5 folds, 6 folds, 7 folds, 8 folds, 9 folds, or 10 folds higher in a cell affected by pulmonary fibrosis compared to a healthy cell. In some instances, the RNA expression level of the transposable element is decreased in a cell affected by pulmonary fibrosis compared to a healthy cell. In some instances, the RNA expression level of the transposable element is at least 10%, 20%, 30%, 40%, 50%, 60%, 70, 80%, or 90% lower than in a cell affected by pulmonary fibrosis compared to a healthy cell.

In some instances, the transposable element is associated with a differential expression of a marker gene of pulmonary fibrosis. In some instances, the marker genes of pulmonary fibrosis include smooth muscle α actin (ACTA2), alpha 1 chain of collagen type I (COL1A1), alpha 1 chain of collagen type 3 (COL3A1), fibroblast activation protein (FAP), fibronectin 1 (FN1), periostin (POSTN), collagen triple helix repeat containing 1 (CTHRC1), alpha 1 chain of collagen type 10 (COL10A1), matrix metalloproteinase 11 (MMP11), thrombospondin 2 (THBS2), latent transforming growth factor beta binding protein 2 (LTBP2), Integrin-α11 (ITGA11), or a combination thereof.

In some instances, the DNA sequence (e.g., genomic DNA sequence) of the transposable element is bound by or bears a binding motif being bound by a pulmonary-fibrosis-related transcription factor. In some instances, the DNA sequence (e.g., genomic DNA sequence) of the transposable element contains a domain or a binding motif that binds to a pulmonary-fibrosis-related transcription factor. In some instances, the binding motif is a common motif or any consensus motif. In some instances, the pulmonary-fibrosis-related transcription factor comprises friend leukemia integration 1 transcription factor (FLI1), interferon regulatory factor 3 (IRF3), interferon regulatory factor 4 (IRF4), (ETS like-1 protein (ELK1), activator protein 1 (AP-1), C-terminal binding protein 2 (CTBP2), Spi-1 Proto-Oncogene (SPI1/PU.1), transcription factor 7 (TCF7), transcription factor 7-like 1 (TCF7L1), transcription factor 7-like 2 (TCF7L2), transcription Factor 21 (TCF21), CCAAT enhancer binding protein beta (CEBPB), forkhead box protein A1 (FOXA1), forkhead box protein A2 (FOXA2), forkhead box protein 04 (FOX04), forkhead box protein L1 (FOXL1), forkhead box protein L2 (FOXL2), forkhead Box M1 (FOXM1), T-box transcription factor 4 (TBX4), T-box transcription factor 5 (TBX5), odd-skipped related 1 (OSR1), early B-cell factor 1 (EBF1), early B-cell factor 2 (EBF2), early B-cell factor 3 (EBF3), twist-related protein 1 (TWIST1), SMAD family member 2 (SMAD2), SMAD family member 3 (SMAD3), nuclear factor-1 (NF1), NK6 homeobox 1 (NKX6-1), NK2 homeobox 1 (NKX2.1), RUNX family transcription factor 2 (RUNX2), POU class 2 homeobox associating factor 1 (POU2AF1), high mobility group AT-hook 2 (HMGA2), lymphoid enhancer-binding factor 1 (LEF1), paired related homeobox 1 (PRRX1), tumor protein p63 (TP63), forkhead box protein P1 (FOXP1), Meis homeobox 1 (MEIS1), TGFβ induced factor homeobox 1 (TGIF1), paired related homeobox 2 (PRRX2), TEA domain family member 1 (TEAD1), TEA domain family member 2 (TEAD2), TEA domain family member 3 (TEAD3), TEA domain family member 4 (TEAD4), signal transducer and activator of transcription 1 (STAT1), signal transducer and activator of transcription 3 (STAT3), signal transducer and activator of transcription 6 (STAT6), nuclear factor-kappa B1 (NFKB1), nuclear factor-kappa B2 (NFKB2), AT-rich interactive domain-containing protein 3A (ARID3A), forkhead box protein S1 (FOXS1), forkhead box protein J2 (FOXJ2), forkhead box protein I1 (FOXI1), forkhead box protein F1 (FOXF1), forkhead box protein E1 (FOXE1), forkhead box protein A3 (FOXA3), forkhead box protein D3 (FOXD3), MYC-Associated Zinc Finger Protein (MAZ), Zinc Finger Protein X-Linked (ZFX), Zinc Finger Protein Y-Linked (ZFY), transcription factor 12 (TCF12), transcription factor AP-2 gamma (TFAP2C), CCCTC-Binding Factor (CTCF), CCCTC-Binding Factor Like (CTCFL or BORIS), Myogenin (MYOG), Nuclear transcription factor Y subunit alpha (NFYA), Nuclear transcription factor Y subunit beta (NFYB), Nuclear transcription factor Y subunit gamma (NFYC), Upstream stimulatory factor 1 (USF1), Upstream stimulatory factor 2 (USF2), Jun Proto-Oncogene/AP-1 Transcription Factor Subunit (JUN), JunD Proto-Oncogene/AP-1 Transcription Factor Subunit (JUND), Fos Proto-Oncogene/AP-1 Transcription Factor Subunit (FOS), FOS Like 1/AP-1 Transcription Factor Subunit (FOSL1), MYC Associated Factor X (MAX), MAF BZIP Transcription Factor F (MAFF), or a combination thereof. In some instances, the DNA sequence (e.g., genomic sequence) of the transposable element is bound by SPI1/PU.1. In some instances, the DNA sequence (e.g., genomic sequence) of the transposable element is bound by EBF1.

In some instances, the transposable element is bound by or targeted by an RNA-binding protein, wherein the transposable element refers to its RNA transcript. In some instances, the RNA-binding protein comprises of DDX3, RBM3, HuR, or a combination thereof.

In some instances, the transposable element is associated with cardiac fibrosis. In some instances, an RNA expression level of the transposable element is altered in a cell affected by cardiac fibrosis compared to a healthy cell (e.g., healthy cardiac cell of the same individual, healthy cardiac cell of a healthy individual). In some instances, the RNA expression level of the transposable element is increased in a cell affected by cardiac fibrosis compared to a healthy cell. In some instances, the RNA expression level of the transposable element is at least 10%, 20%, 30%, 40%, 50%, 60%, 70, 80%, or 90% more than in a cell affected by cardiac fibrosis compared to a healthy cell. In some instances, the RNA expression level of the transposable element is at least 1.5 folds, 2 folds, 3 folds, 4 folds, 5 folds, 6 folds, 7 folds, 8 folds, 9 folds, or 10 folds higher in a cell affected by cardiac fibrosis compared to a healthy cell. In some instances, the RNA expression level of the transposable element is decreased in a cell affected by cardiac fibrosis compared to a healthy cell. In some instances, the RNA expression level of the transposable element is at least 10%, 20%, 30%, 40%, 50%, 60%, 70, 80%, or 90% lower than in a cell affected by cardiac fibrosis compared to a healthy cell. In some instances, the transposable element is associated with a differential expression of a marker gene of cardiac fibrosis. In some instances, the marker genes of cardiac fibrosis include smooth muscle α actin (ACTA2), alpha 1 chain of collagen type I (COL1A1), alpha 1 chain of collagen type 3 (COL3A1), fibroblast activation protein (FAP), fibronectin 1 (FN1), periostin (POSTN), collagen triple helix repeat containing 1 (CTHRC1), alpha 1 chain of collagen type 10 (COL10A1), matrix metalloproteinase 11 (MMP11), thrombospondin 2 (THBS2), latent transforming growth factor beta binding protein 2 (LTBP2), Integrin-α11 (ITGA11), or a combination thereof.

In some instances, the transposable element is associated with dermal fibrosis. In some instances, an RNA expression level of the transposable element is altered in a cell affected by dermal fibrosis compared to a healthy cell. In some instances, the RNA expression level of the transposable element is increased in a cell affected by dermal fibrosis compared to a healthy cell. In some instances, the RNA expression level of the transposable element is at least 10%, 20%, 30%, 40%, 50%, 60%, 70, 80%, or 90% more than in a cell affected by dermal fibrosis compared to a healthy cell. In some instances, the RNA expression level of the transposable element is at least 1.5 folds, 2 folds, 3 folds, 4 folds, 5 folds, 6 folds, 7 folds, 8 folds, 9 folds, or 10 folds higher in a cell affected by dermal fibrosis compared to a healthy cell. In some instances, the RNA expression level of the transposable element is decreased in a cell affected by dermal fibrosis compared to a healthy cell. In some instances, the RNA expression level of the transposable element is at least 10%, 20%, 30%, 40%, 50%, 60%, 70, 80%, or 90% lower than in a cell affected by dermal fibrosis compared to a healthy cell. In some instances, the transposable element is associated with a differential expression of a marker gene of dermal fibrosis. In some instances, the marker genes of dermal fibrosis include smooth muscle α actin (ACTA2), alpha 1 chain of collagen type I (COL1A1), alpha 1 chain of collagen type 3 (COL3A1), fibroblast activation protein (FAP), fibronectin 1 (FN1), periostin (POSTN), collagen triple helix repeat containing 1 (CTHRC1), alpha 1 chain of collagen type 10 (COL10A1), matrix metalloproteinase 11 (MMP11), thrombospondin 2 (THBS2), latent transforming growth factor beta binding protein 2 (LTBP2), Integrin-α11 (ITGA11), or a combination thereof.

In some instances, the transposable element associated with fibrosis belongs to ERV1, ERV2, or MaLR families. In some instances, the transposable element associated with fibrosis belongs to transposable elements identified in Table 1 or Table 2. In some instances, the transposable element belongs to LTR16, LTR16A, LTR16A1, LTR16A2, LTR16B, LTR16B1, LTR16B2, LTR16C, LTR16D, LTR16D1, LTR16D2, LTR16E1, LTR16E2, ERV3-16A3_LTR, MER39, THE1A, THE1B, THE1C, THE1D, MSTA, MSTA1, MSTB, MSTB1, MSTB2, MSTC, MSTD, MLT1A0, MLT1A1, MLT1C, MLT1D, MLT1E, MLTIEI, MLT1E2, MLT1F, MLT1F1, MLT1F2, MLT1G1, MLT1G3, MLT1H, MLT1H1, MLT1I, MLT1J, MLT1J1, MLT1J2, MLT1K, MLT1L, MLT1M, MLT1N2, MLT1O, MLT2A1, MLT2A2, MLT2B1, MLT2B2, MLT2B3, MLT2B4, MLT2B5, MLT2C1, MLT2C2, MLT2D, MLT2E, MLT2F, MLT1A, MLT1B, MLT1E1A, MLT1E3, MLT1G, MLT1H2, MLT1C2, MLT1F-int, MST-int, MLT-int, MLT1H-int, MLT1J-int, THE1-int, HERV16, ERVL-E, HERVL, ERVL-B4, ERV3-16A3_I, HERV3, HERVK3, HERVL18, LTR10A, LTR10E, LTR24C, LTR26, LTR40c, LTR44, LTR47B4, LTR8B, LTR90A, MamGypsy2-I, MamRep605, MER21C, MER41B, MER57E3, MER68, MER77B, THE1D-int, LTR33, LTR50, LTR78, LTR41, LTR41B, HERV3-int, LTR13, HERVL18-int, MER66C, MER66D, or LTR19C subfamilies.

In some instances, the transposable element transcript is transcribed from a sequence comprising at least 60%, at least 70%, at least 80%, at least 90%, or at least 95% identical to at least a portion of a consensus sequence selected from any one of SEQ ID NOs: 4, 7-11, and 14-121. In some instances, the transposable element associated with fibrosis comprises a sequence that is at least 60%, at least 70%, at least 80%, at least 90%, or at least 95% identical to at least a portion of a sequence selected fromSEQ ID NOs: 4, 7-11, and 14-121. In some instances, the transposable element transcript is transcribed from a sequence comprising at least 60%, at least 70%, at least 80%, at least 90%, or at least 95% identical to at least a portion of a consensus sequence selected from any one of SEQ ID NOs: 4, 10, 14, 15, 18, 27-32, and 34-38. In some instances, the transposable element associated with fibrosis comprises a sequence that is at least 60%, at least 70%, at least 80%, at least 90%, or at least 95% identical to at least a portion of a sequence selected from SEQ ID NOs: 4, 10, 14, 15, 18, 27-32, and 34-38. In some instances, the transposable element associated with fibrosis comprises a sequence of at least 50%, 60%, at least 70%, at least 80%, at least 90%, or at least 95% identical to SEQ ID NO: 4 or a portion thereof. In some instances, “a portion” as used herein refers to any segments that take up at least 10%, 20%, 30%, 40%, 50%, 60%, 70%, 80%, or 90% the full sequence.

In some instances, the transposable element associated with fibrosis comprises a sequence of SEQ ID NO: 5. In some instances, the transposable element associated with fibrosis is located around chr3: 45818666-45818847. In some instances where the transposable element associated with fibrosis is located around chr3: 45818666-45818847, the transposable element associated with fibrosis aligns with the 299th to 489th base pairs of its consensus sequence (see SEQ ID NO: 4) with about 70% identity and gaps that take up about 8% of the sequence of the transposable element. In some instances, the transposable element associated with fibrosis comprises a sequence of SEQ ID NO: 6. In some instances, the transposable element is located around chr21: 42041909-42042242.

TABLE 1
Table of Sequences Disclosed

| SEQ ID | | |
|---|---|---|
| No | Sequences | |
| 1 | 5′-CGGAGGCATGAGGTAG-3′ | ASO-1 targeting |
| | | LTR16C on |
| | | chr3: 45,818,666- |
| | | 45,818,847 |
| 2 | 5′-TGAGCAGGTTAGCACT-3′ | ASO-2 targeting |
| | | LTR16C on |
| | | chr3: 45,818,666- |
| | | 45,818,847 |
| 3 | 5′-CTACCTCATGCCTCCG-3′ | ASO-3 targeting |
| | | LTR16C on |
| | | chr3: 45,818,666- |
| | | 45,818,847 |
| 4 | TGTAGCAGACGCTGTCGGTGCCCCGCCCATATCCCCTCGGCCCTTA | Consensus sequence |
| | CCATTTCAGTGCACGCCGGCCCGACTTCCAACTGCCAGCACCTGCA | for transposable |
| | TCTCTTTGCCTGAGGGCTTTCTCTGGCCGCCGGAGCCCGCTCTGCC | elements that belong |
| | CACGCGCANGGCAGGCCGGAAGTGCCGGGGAATTAACGCCCCCCG | to LTR16C |
| | GGAGCAGCCCTCAACCAATGACTGACGGGAGTTGGTGNATAAATA | subfamily |
| | CCCCAGCTCCCTCGCCCCTCGGGTGGGATAACTCTGAGGCGCGTGT | |
| | TCTACACTGTCTCCCAGAGTTCCCCAGCGGGATTGAGCTCCAGTTG | |
| | CCCACAGTGGTAACTTGCTCGATAACGCACCCTTTATTGGCTNCCT | |
| | TCCCTTCCCTGTCTCACTTCCCCACTCCCCTACCGGTGTTTCCTGGG | |
| | ATCACCTCCCAAATAAACTACTTGCACTCGAATCCTTGTCTCAGGG | |
| | TCTGCTTCTGGGGGAACCCAAACTAAGACA | |
| 5 | TCAGTGGGATTGAGCGTGGTGGTCCACAGTGCTAACCTGCTCATTA | LTR16C within |
| | ACACACATTTTATCTCCTTTCTTCCCTTTCCTACCTCATGCCTCCGT | chr3: 45,818,666- |
| | ACCCTCACCCCACTTTCTGGGATCCCCTCCCCACTTCAACCAAGAT | 45,818,847 |
| | CCCTGCCTCAGGGTTTTGCTTTTGGGGGAGCTGAAATAAAACA | |
| 6 | CTGCCTGTCTTTCCTGAAGACGTTCTCTCTCTGGCCCTGTTCCCAGG | LTR16E1 within |
| | GCAGGCTGAAGGACAAAGGACAGGAAGGTAAGGTCCCAGGAGTG | chr21: 42,041,909- |
| | ACCCCCAGTGTGGAGGATGAGAGTTGTTGGATAAATGTCCCAGCT | 42,042,242 |
| | CCCTCCTCCCTCCATGGGGCAGTTCTGGGGCATGTTCTGCTCAGAC | |
| | CTCAATGCAGTCCCAGTGGGATGAACCGAAGTTCCCTCTGGCAGG | |
| | GATCCTCTCTCTAGGGTACCTGCACTCCTGCCTTCACTGTGCTTCCT | |
| | GAGATCACCCCCAAAATCAACTGCTTGCCCCCAAATCTTTGTCATA | |
| | GGGTTTTCAAGAGA | |
| 7 | TGTAGCAGACGCCTCTGGTGCCCCGCGTCACATCCCCTCGGCCCAC | Consensus sequence |
| | CTCTGATTTCAGCCGCAGCTGCGGTGGACAGTTCCGTGCGAGCTCA | for transposable |
| | GACTCACCTTTGCTGACAGCGTCCCACCTCAAGCGCGCGCCGTGCG | elements that belong |
| | TCTTTCTGCTTTCTGCCCCAGGGCCTTCTCCGACGCCGCGGGAGCC | to LTR16E1 |
| | CGCTCGGCCCGCGCGCAAGCGCAGCCCGGAAGTGCGGGGGAGTTA | subfamily |
| | ACGCCCCCGGGGGCAACCCTCAACCAATGGGGGACGGGAGCCGGT | |
| | GGATAAATGCCCCAGCCTCCCGTCCTTCAGGTGGACAATTCTGGGA | |
| | GGCATTCTGTACGCTTCTCAGGAGGTCCCAGCGGGATCGAGCCCC | |
| | NGTTGCCCACAGCGGCGACCTCGATAACGCACCCTTATATTGGCTT | |
| | TTCCTCCTTCCCTGTCTCACTTCCCCGCTCCCTCACTCCTGCTTCCT | |
| | GGGATCACCTCCCAAATAAACTACCTGCACCCAAGTCCTTGTCTCA | |
| | GGCTCTGCTTTCGGGGGAACCCAAACTAAGACAG | |
| 8 | TGATATGGTTTGGCTGTGTCCCCACCCAAATCTCATCTTGAATTGT | Consensus sequence |
| | AGCTCCCATAATTCCCACGTGTCGTGGGAGGGACCCGGTGGGAGG | for transposable |
| | TAATTGAATCATGGGGGCGGGTCTTTCCCGTGCTGTTCTCGTGATA | elements that belong |
| | GTGAATAAGTCTCACGAGATCTGATGGTTTTATAAAGGGGAGTTCC | to THE1B subfamily |
| | CCTGCACANGCTCTCTTGCCTGCCGCCATGTAAGACGTGNCTTTGC | |
| | TCCTCCTTCGCCTTCCGCCATGATTGTGAGGCCTCCCCAGCCACGT | |
| | GGAACTGTGAGTCCATTAAACCTCTTTCCTTTATAAATTACCCAGT | |
| | CTCGGGTATGTCTTTATCAGCAGCGTGAAAACGGACTAATACA | |
| 9 | GTAAATTGGTACCAGTAGAGTGGGGCGCTGCTGAAAAGATACCCG | Consensus sequence |
| | AAAATGTGGAAGCGACTTTGGAACTGGGTAACAGGCAGAGGTTGG | for transposable |
| | AACAGTTTGGAGGGCTCAGAAGAAGACAGGAAAATGTGGGAAAG | elements that belong |
| | TTTGGAACTTCCTAGAGACTTGTTGAATGGCTTTGACCAAAATGCT | to THE1-int |
| | GATAGTGATATGGACAATAAAGTCCAGGCTGAGGTGGTCTCAGAT | subfamily |
| | GGAGATGAGGAACTTGTTGGGAACTGGAGCAAAGGTGACTCTTGT | |
| | TATGTTTTAGCAAAGAGACTGGCGGCATTTTGCCCCTGCCCTAGAG | |
| | ATTTGTGGAACTTTGAACTTGAGAGAGATGATTTAGGGTATCTGGC | |
| | GGAAGAAATTTCTAAGCAGCAAAGCATTCAAGAGGTGACTTGGGT | |
| | GCTGTTAAAGGCATTCAGTTTTANAAGGGAAGCAGAGCATAAAAG | |
| | TTCGGAAAATTTGCAGCCTGACGATGCGATAGAAAAGAAAANCCC | |
| | ATTTTCTGAGGAGAAATTCAAGCCGGCTGCAGAAATTTGCATAAG | |
| | TAACGAGGAGCCGAATGTTAATCCCCAAGACAATGGGGAAAATGT | |
| | CTCCAGGGCATGTCAGAGGCCTTCGCGGCAGCCCCTCCCATCACA | |
| | GGCCCGGAGGCCTAGGAGGAAAAAATGGTTTCGTGGGCCGGGCCC | |
| | AGGGTCCCCGTGCTGTGTGCAGCCTAGGGACTTGGTGCCCTGCGTC | |
| | CCAGCCGCTCCAGCCGTGGCTGAAAGGGGCCAACGTACAGCTCGG | |
| | GCCGTGGCTTCAGAGGGTGCAAGCCCCAAGCCTTGGCAGCTTCCA | |
| | CGTGGTGTTGAGCCTGCGGGTGCACAGAAGTCAAGAATTGAGGTT | |
| | TGGGAACCTCCGCCTAGATTTCAGAGGATGTATGGAAACGCCTGG | |
| | ATGCCCAGGCAGAAGTTTGCTGCAGGGGCGGGGCCCTCATGGAGA | |
| | ACCTCTGCTAGGGCAGTGCGGAAGGGAAATGTGGGGTCGGAGCCC | |
| | CCACACAGAGTCCCTACTGGGGCACCGCCTAGTGGAGCTGTGAGA | |
| | AGAGGGCCACCGTCCTCCAGACCCCAGAATGGTAGATCCACCGAC | |
| | AGCTTGCACCGTGCGCCTGGAAAAGCCGCAGACACTCAACGCCAG | |
| | CCCGTGAAAGCAGCCGGGAGGGAGGCTGTACCCTGCAAAGCCACA | |
| | GGGGCGGAGCTGCCCAAGACCATGGGAACCCACCTCTTGCATCAG | |
| | CGTGACCTGGATGTGAGACATGGAGTCAAAGGAGATCATTTTGGA | |
| | GCTTTAAGATTTGACTGCCCCGCTGGATTTCGGACTTGCATGGGGC | |
| | CTGTAGCCCCTTTGTTTTGGCCAATTTCTCCCATTTGGAATGGCTGT | |
| | ATTTACCCAATGCCTGTACCCCCATTGTATCTAGGAAGTAACTAAC | |
| | TTGCTTTTGATTTTACAGGCTCATAGGCGGAAGGGACTTGCCTTGT | |
| | CTCAGATGAGACTTTGGACTGTGGACTTTTGAGTTAATGCTGAAAT | |
| | GAGTTAAGACTTTGGGGGACTGTTGGGAAGGCATGATTGGTTTTG | |
| | AAATGTGAGGACATGAGATTTGGGAGGGGCCAGGGGCGGAA | |
| 10 | TGTAGTGGATGCTGTGGTGCGCCGCCCAGATCGNNCCCCCCTTCAG | Consensus sequence |
| | GACCGAGGCACTCATTCCCCCAGCTGCCGGGAGTGTTGGCTGCTG | for transposable |
| | ACGGCTCNCAGCTGAGTCCCTCTCCGGGAATTGCCCTCGGCCGAA | elements that belong |
| | GGGAGCTGCCTCGCCCAAGGTTACGCCCCCTTCCCGGGGGCAGCC | to LTR16 subfamily |
| | CGCATCCAATGACTGGTCGATGCGGGGGTACAAAGGCCTCAGTCC | |
| | TCAATTCGGGACAACTCTGAAGGGCCATCCCAGCTCCAGAGCTCC | |
| | CCGTGGGATCGGCTGAGGCCTCTGTTGCGACTGCATCGCAGTTCAA | |
| | CTTCTCCCTCTGCCCAATCCTGCTTCCTTCACTCCCTCACAGGTGTT | |
| | GNTCCCGAGAGCACTCCCCAATAAACCTCCTGCACGCAAATCTCC | |
| | GTCTCAGAGTCTGTTTCCCGGGGAACCCA | |
| 11 | CATGAGTGGTACCGGGAGTGGTCCGNAGAAAACAGGCGGTAAGAT | Consensus sequence |
| | GGGGTTTTGGGACTGGCTCACTCACCGCCCGGCGGACAGAGGATA | for transposable |
| | CCATCCTGGCACAAGGTGGCGGCCCAGTTGCTAAAGATTTCACCG | elements that belong |
| | GTGGTGACCTGGGAAAATGTGTCCCAGNNNTTGGAAGGGGAATGC | to ERV3-16A3_I |
| | CCTTGCAGGTGCAATGATTCAGGCATTTGAAAGATATGGGGAGAN | subfamily |
| | AATGCCTACAAGGACAGCGGAGTTGGCTGGTTATTGCTAAGTTGT | |
| | ATTGATGCCCTGCAGAGGGATAATGAGAAACTGAGGGCNGTTAAC | |
| | AAGCAGTTAAAGGCTAAGTGTGAGAGCCAGAGGGCCTCTTTGNTG | |
| | GTAGCTTACAAAGAGGCCCTTATCTCCTGCAGTGGAAGAGCAGAC | |
| | ANAGCTGAGGAGCAGACTCAGGATCTAATAGTTAGAGTCGCAGAG | |
| | CTCCAGAGACGTTTGAATGCTCAGCCAAGGCGAGGTCTGTTATGCT | |
| | AAAGNTAAGGCCCTTGGTTGGGAAAACCTGGGACGGAACACGGG | |
| | ATGGGGACATCTGGGTGGATGCCCCCGAAGATNTTGGCTCTCCAG | |
| | ACTCCTCTGAACCCTCNGAGCCTGCAGAAGTGGCCCACCCCTCCCT | |
| | ANTAAGAGCTAGCACTCCTCTTGCGCCCGCGCTGGAAGACGNTGC | |
| | AGAGGCCTCTCCCCGCAAGGCAACAGGTGCCCCCTCTCAGGANCT | |
| | GCCCCCACCTCCCCTCCTGGCCGCCAGGCCGATAACTAGGGTTAA | |
| | GTCNCAGCATAACCCGGCTGGGGACGTGCTGGGCCTGATAAGGGA | |
| | GGAAAGGGACTATACCCCAAAGGAGCTGCAAGAATTAGCCAGCAT | |
| | GTACCGGCAGGAGCCAGGGGAGTATACNCNTGGGACTGGATTTTG | |
| | AGGGTGCTTGATCAAGGGGGCCGGAATATAAGCGGTAAGACTGGA | |
| | TAANGGAAGAATTTATTGACTTGGGAGCACTTTCTCGGGATACAG | |
| | GATTTAACACCCTGGCAAGGACCCCAGGAGATGGTGCAAACTCGC | |
| | TGCTAGGATGGCTCCTAGAAGCGAGAAAAATGGCCCACGCTGAGC | |
| | GAAGTNGAAATGCCNGAATTGCCNTGGCAGACGGTAGAGGAAGG | |
| | GATTAAAGGCTCAGGGAAGTGGGCATGCTGGAATGGATATNATTA | |
| | TGTAAGGCCGGAAGACCCACCAGANGATTATGTTCCACGGGAGGG | |
| | CCCACACCATTCACCAAGGCCATNAGGAATGCGCTGGTGAGAGGG | |
| | GCACCAGCANNTCACTAAGAAGTTCAGTGGTGGCTCTCCTCTGCA | |
| | GGCCAGGGCTGACGGTAGGAGAGGCCGTTACAGAACTGGGCTCGC | |
| | TGATAGCAATGGGGATGATAGGACCCCGAAACAATAGAGGCCAG | |
| | GTGGCGGCGCTTAACCGCCAGAAGCCAGGNGGNCGCAATTATCGT | |
| | AATGACCGGAAGGTCGGAGTGGCAGCCAAGGGGGCCTGACCCAA | |
| | GAGAGTTGTGGAGATGGTTAATAGAACACGGCGTCCCTAGGGGCA | |
| | AAATAGATNGACAAGGGTGCTGCTTAACTTGTAACAGCAGAAAAA | |
| | ATCTGGGAGGCTGAGGGCGGTCGCCCCAATAAAAAAGTCACGATC | |
| | CCTTGCCCAGTTTCCGGACCTGAGCCAGTTTTCAGACCCGGAACCC | |
| | ATTGACTGAAGAGGNGGCCGGGTCCCCAGGAGGAAGGACACTGC | |
| | AATGATTCTTCTCNTTCCTCCCGNAGGGACCTATGGCCATTTACTC | |
| | GGGTAACTGTACACTGGGGAAAGGGAATACCCAGACATTTCGAGG | |
| | ACTGTTGGACACAGGGTCTGAGTTGACATTGATACCCGGAGACCC | |
| | GAAGCGTCATCATGGCCCCCTGTTAGAGTGGGGGCATATGGGGGC | |
| | CAGGTAATAAATGGAGTCCTGGCCAAGGTCCGGCTCACAGTGGGT | |
| | CCACTGGGTCCANGAACCCAGTGGTCATTTCCCCGGTCCCCGAATG | |
| | TATAATTGGGTAGTTGGAGTAACCCCCACATTGGNTCCTTGGCCTG | |
| | TGGGGTAAGAGCTATCATAGTGGGGAAGGCCAAGTGGAAGCCTCT | |
| | GAAACTGCCCNCCNCCNCCATTCCTAGCCAAGATAGTAAATTATTA | |
| | TATCCTCTCGAGGAAGTGGCGGATGCAGATTAGTGCCACCNTTAA | |
| | AGACCTAAAGGATGCAGGGGTGGTGGTCCCCATCATATCTCCATTT | |
| | AATTCACCAGTCTGGCCCCTGCAGAAACCGGATGGATCCTGGAGA | |
| | ATGACNGTAGACTACCGCAAACTCAACCAAGTAGTAGCCCCGATT | |
| | GCAGCTGCTGTGCCAGATGTGGTATCTTTGCTAGAGCAGATTAACA | |
| | CGGCCTCAGGTACATGGTATGCGGCCATTGATTTGGCGAATGCGTT | |
| | CTTTTCCATTCCNATCAGAAAAGAGGATCAGAAACATTCACATGA | |
| | ACAGGAAANAATATACATTTACAGTTTTGCCCCAGGGCTATGTTAT | |
| | CTTCTCCTCTGTCATAATAAGTCCGAAGAGATCTGGACCGTCTGGA | |
| | CATCCCGCAGAACATCACATTGATCCATTACATCGATGACATCATG | |
| | CTGATCGGACNGGATGAGCAAGAGGTGGCTACGTTAGGCCTTGGT | |
| | AAGACACATGCGCTGAGGGTGGAGATAACCCTACGAAGATTCAGC | |
| | GNNNGGACCTGCCACNTCAGTAAAGTTTTTAGGGGTCCAGTGGTC | |
| | NGGGGCATGCCGGGACATCCCCTCCAAAGTAAAAGACAAATTGTT | |
| | GCATCTTGCATCTCCTACCACGAAGAAGGAAGCACAACGCCTGGT | |
| | AGGCCTCTTTGGGTTCTGGAGGCAGCATATTCCACACCTGGGAATA | |
| | CTGCTCCGGCCCATNTACCGGGTGACACGGAAGGCTGCCAGCTTT | |
| | GAGTGGGCCCAGAGCAGGAAAGGGCTCTGCAGCAGGTCCAGGCTG | |
| | CGGTGCAAGCAGCCCTGCCGCTTGGGCCATACGACCCGGCAGACC | |
| | CTATGGTGTTGGAGGTGTCAGTGGTGGGAAAAGATGCNGTGTGGA | |
| | GTTTATGGCAAGCCCCAGTGGGAGAATCACAACGCAGGCCCCTGG | |
| | GGTTCTGGAGCAAGGCTGCAAGTCTTTTGAAAAACAGCTCTTNNN | |
| | ATGCTACTGGTAGAGANGGAACGCTTACCATGGGACACCAAGTGA | |
| | CACGNANCAAAATGTACCCCGTCATGAGCTGGGTTCTGTCGGACC | |
| | CACCAAGTCATAAGGTCGGGCGGGCCCAGCAGCAATCCATCGTAA | |
| | GATGGAAGTGGTACATCCGGGATCGAGCACGAGGAGGACCACAA | |
| | GTAAGCTGCATGAGCAGGTAGCCCAGACCCCCATGTCACCCACCA | |
| | CNGTTGCACCAGCGCTCCTTCAGCTCACACCTATGGCCGTATGGAG | |
| | NAGAGGTGTGNTCCTGATCGACCAGCTGAAGGAGGAGGAAAAAN | |
| | GCCCGAGCTTGGTTCACGGATGGGTCGGCTCGGTATGTGGGTGCA | |
| | AGCCGAAAATGGACGGCGGCTTGCACTACAGCCNCACTCAGGGGT | |
| | GGCCTTGAAAGACAGTGGNGAGGGAAAATCTTCCCAATGGGCAGA | |
| | GCTTCGGGCGGTGCACCTGGTCATCCACTTTGTGTGGAAGGAGAA | |
| | GTGGCCCGAGGTNAGAATATATACGGACTCATGGGCAGTGGCGAA | |
| | TGGCTTGGCCGGCTGGTCAGGGGCCTGGAAGGAGAAAGATTGGAA | |
| | GATCGGAGACAAGGAGGTCTGGGGANTAGAGGCATGTGGATGGA | |
| | CNTATGGGAGTGGGCACGAAGTGTGAAGATCTTTGTATCACATGTT | |
| | AACGCCCACCAGAGAGCATCCACCACGGAAGAGGCACTAAACAA | |
| | CCAAGTAGACAAAATGACTCGGCCAGTTGACGTCAGCCAGCCTCT | |
| | GTCATCGGCCACCCCANGTGCTGGCACAATGGGCACATGAACGGA | |
| | GTGGCCACGGTGGCAGAGATGGAGGCTACGCATGGGCCCAACAGC | |
| | ATGGACTCCCACTCACCAAGGCTGATCTAGCTACTTGCTGCCNCNN | |
| | CTGAATGTCCAACCTGCCAGCAACAGAGACCGATCCCCGATATGG | |
| | CACCATTCCTCGAGGAGACCAACCAGCCACTTGGTGGCAAGTTGA | |
| | CTACATTGGGCCCCTTCCATCCTGGAAGGGNCAGCGGTTCATCCTN | |
| | ACAGGAATACATATTCCGGGTATGGGTTTGCCTTTCCTGCCCGCAG | |
| | GGCCTCAGCCAGCACCACTATCCGAGGGCTTACGGAGTGCCTGAT | |
| | CCACNGGCATGGGATCCCACACAACATNGCATCNGACCAGGGACC | |
| | CACTTTACAGCAAAGGAGGTGCGGGAGTGGGCCCATGACCATGGG | |
| | ATCCACTGGTCNTATCACATACCGCACCATCCAGAAGCTGCCGGCC | |
| | TGATAGAGCGNTGGAACGGCCTNCTGAAGGCACAGCTGAAGCGCC | |
| | AGCTCGGAGACGATACTCTGCGAGGATGGGGCGCCATCCTTCAGG | |
| | ATGCAGTGTCCCCAATAGGAAGAATACATGGGTCCGGGAACCAAG | |
| | GGGTGGAAGCAGGAGTGTNGCCCCACTTACCATCACTCCCAGTGA | |
| | CCCACTGGGGGANTTTGTGCTTCCCGTCCCCGCAACTCTGGGCTCT | |
| | GCAGGNTTAGAGGTCCTGGTCCCCAAAGGGGGNACGCTCTCGCCA | |
| | GGGGACACAGCGTCCCATTGAACTNTANAGCTACGGCTGCCGCCT | |
| | GGGCACTTTGGGCTCCTTGTGTCCAGGGACCAGCAGGCAAGAAGA | |
| | GGAGTCACCATCTTGGCAGGGGTAATTGACCCTGATCATCAGGAG | |
| | GAGGTAGGGCTGCTNTTACACAATGGGGGCAGGGAGGAATACGTN | |
| | TGGAACCCAGGTGATCCACTTGGGCGCCTCTTGGTACTCCCTTGCC | |
| | CAATTNTGACTGTAAATGGACAAGTGCAGCAACCCCGGCCTGAGA | |
| | AGGGCATGGTNACCAGGGGCTCAGACCCCTCAGGAATGAGGGTCT | |
| | GGGTCACGCCACCAGGTAAGCCACCGAGACCAGCAGAGTGNTAGC | |
| | TGAGGGTGAGGGGAATCTAGAATGGATAGTGGAGGAGGGAGACG | |
| | ATGAGTATCAGTTGCGGCCCCGAGACCAACTGAGCGACGGGGGCT | |
| | GTAGTTCGTCCCACTAACCTCCCTCTTCTAAGTTTCCCTCAGGAAG | |
| | AGAGGCCCACNGGAATCCTGGAGGAGCTGCTCCTGAACCGAACGT | |
| | GTATGGAGAAGTGGATCCGNGCGGCGCAAGGGGTGGAC | |
| 12 | CCACAGTGCTAACCTGCTCA | Primer for LTR16C |
| | | within |
| | | chr3: 45,818,666- |
| | | 45,818,847 |
| 13 | TCCCCCAAAAGCAAAACCCT | Primer for LTR16C |
| | | within |
| | | chr3: 45,818,666- |
| | | 45,818,847 |
| 14 | CATGAGTGGTACCGGGAGTGGTCCGNAGAAAACAGGCGGTAAGAT | Consensus sequence |
| | GGGGTTTTGGGACTGGCTCACTCACCGCCCGGCGGACAGAGGATA | for transposable |
| | CCATCCTGGCACAAGGTGGCGGCCCAGTTGCTAAAGATTTCACCG | elements that belong |
| | GTGGTGACCTGGGAAAATGTGTCCCAGNNNTTGGAAGGGGAATGC | to ERV3-16A3_I |
| | CCTTGCAGGTGCAATGATTCAGGCATTTGAAAGATATGGGGAGAN | subfamily |
| | AATGCCTACAAGGACAGCGGAGTTGGCTGGTTATTGCTAAGTTGT | |
| | ATTGATGCCCTGCAGAGGGATAATGAGAAACTGAGGGCNGTTAAC | |
| | AAGCAGTTAAAGGCTAAGTGTGAGAGCCAGAGGGCCTCTTTGNTG | |
| | GTAGCTTACAAAGAGGCCCTTATCTCCTGCAGTGGAAGAGCAGAC | |
| | ANAGCTGAGGAGCAGACTCAGGATCTAATAGTTAGAGTCGCAGAG | |
| | CTCCAGAGACGTTTGAATGCTCAGCCAAGGCGAGGTCTGTTATGCT | |
| | AAAGNTAAGGCCCTTGGTTGGGAAAACCTGGGACGGAACACGGG | |
| | ATGGGGACATCTGGGTGGATGCCCCCGAAGATNTTGGCTCTCCAG | |
| | ACTCCTCTGAACCCTCNGAGCCTGCAGAAGTGGCCCACCCCTCCCT | |
| | ANTAAGAGCTAGCACTCCTCTTGCGCCCGCGCTGGAAGACGNTGC | |
| | AGAGGCCTCTCCCCGCAAGGCAACAGGTGCCCCCTCTCAGGANC | |
| | TGCCCCCACCTCCCCTCCTGGCCGCCAGGCCGATAACTAGGGTTAA | |
| | GTCNCAGCATAACCCGGCTGGGGACGTGCTGGGCCTGATAAGGGA | |
| | GGAAAGGGACTATACCCCAAAGGAGCTGCAAGAATTAGCCAGCAT | |
| | GTACCGGCAGGAGCCAGGGGAGTATACNCNTGGGACTGGATTTTG | |
| | AGGGTGCTTGATCAAGGGGGCCGGAATATAAGCGGTAAGACTGGA | |
| | TAANGGAAGAATTTATTGACTTGGGAGCACTTTCTCGGGATACAG | |
| | GATTTAACACCCTGGCAAGGACCCCAGGAGATGGTGCAAACTCGC | |
| | TGCTAGGATGGCTCCTAGAAGCGAGAAAAATGGCCCACGCTGAG | |
| | CGAAGTNGAAATGCCNGAATTGCCNTGGCAGACGGTAGAGGAAG | |
| | GGATTAAAGGCTCAGGGAAGTGGGCATGCTGGAATGGATATNATT | |
| | ATGTAAGGCCGGAAGACCCACCAGANGATTATGTTCCACGGGAGG | |
| | GCCCACACCATTCACCAAGGCCATNAGGAATGCGCTGGTGAGAGG | |
| | GGCACCAGCANNTCACTAAGAAGTTCAGTGGTGGCTCTCCTCTGC | |
| | AGGCCAGGGCTGACGGTAGGAGAGGCCGTTACAGAACTGGGCTCG | |
| | CTGATAGCAATGGGGATGATAGGACCCCGAAACAATAGAGGCCAG | |
| | GTGGCGGCGCTTAACCGCCAGAAGCCAGGNGGNCGCAATTATCGT | |
| | AATGACCGGAAGGTCGGAGTGGCAGCCAAGGGGGCCTGACCCAA | |
| | GAGAGTTGTGGAGATGGTTAATAGAACACGGCGTCCCTAGGGGCA | |
| | AAATAGATNGACAAGGGTGCTGCTTAACTTGTAACAGCAGAAAAA | |
| | ATCTGGGAGGCTGAGGGCGGTCGCCCCAATAAAAAAGTCACGATC | |
| | CCTTGCCCAGTTTCCGGACCTGAGCCAGTTTTCAGACCCGGAACCC | |
| | ATTGACTGAAGAGGNGGCCGGGTCCCCAGGAGGAAGGACACTGC | |
| | AATGATTCTTCTCNTTCCTCCCGNAGGGACCTATGGCCATTTACTC | |
| | GGGTAACTGTACACTGGGGAAAGGGAATACCCAGACATTTCGAGG | |
| | ACTGTTGGACACAGGGTCTGAGTTGACATTGATACCCGGAGACCC | |
| | GAAGCGTCATCATGGCCCCCTGTTAGAGTGGGGGCATATGGGGGC | |
| | CAGGTAATAAATGGAGTCCTGGCCAAGGTCCGGCTCACAGTGGGT | |
| | CCACTGGGTCCANGAACCCAGTGGTCATTTCCCCGGTCCCCGAATG | |
| | TATAATTGGGTAGTTGGAGTAACCCCCACATTGGNTCCTTGGCCTG | |
| | TGGGGTAAGAGCTATCATAGTGGGGAAGGCCAAGTGGAAGCCTCT | |
| | GAAACTGCCCNCCNCCNCCATTCCTAGCCAAGATAGTAAATTATTA | |
| | TATCCTCTCGAGGAAGTGGCGGATGCAGATTAGTGCCACCNTT | |
| | AAAGACCTAAAGGATGCAGGGGTGGTGGTCCCCATCATATCTCCA | |
| | TTTAATTCACCAGTCTGGCCCCTGCAGAAACCGGATGGATCCTGGA | |
| | GAATGACNGTAGACTACCGCAAACTCAACCAAGTAGTAGCCCCGA | |
| | TTGCAGCTGCTGTGCCAGATGTGGTATCTTTGCTAGAGCAGATTAA | |
| | CACGGCCTCAGGTACATGGTATGCGGCCATTGATTTGGCGAATGC | |
| | GTTCTTTTCCATTCCNATCAGAAAAGAGGATCAGAAACATTCACAT | |
| | GAACAGGAAANAATATACATTTACAGTTTTGCCCCAGGGCTATGTT | |
| | ATCTTCTCCTCTGTCATAATAAGTCCGAAGAGATCTGGACC | |
| | GTCTGGACATCCCGCAGAACATCACATTGATCCATTACATCGATGA | |
| | CATCATGCTGATCGGACNGGATGAGCAAGAGGTGGCTACGTTAGG | |
| | CCTTGGTAAGACACATGCGCTGAGGGTGGAGATAACCCTACGAAG | |
| | ATTCAGCGNNNGGACCTGCCACNTCAGTAAAGTTTTTAGGGGTCC | |
| | AGTGGTCNGGGGCATGCCGGGACATCCCCTCCAAAGTAAAAGACA | |
| | AATTGTTGCATCTTGCATCTCCTACCACGAAGAAGGAAGCACAAC | |
| | GCCTGGTAGGCCTCTTTGGGTTCTGGAGGCAGCATATTCCACACCT | |
| | GGGAATACTGCTCCGGCCCATNTACCGGGTGACACGGAAGGCT | |
| | GCCAGCTTTGAGTGGGCCCAGAGCAGGAAAGGGCTCTGCAGCAGG | |
| | TCCAGGCTGCGGTGCAAGCAGCCCTGCCGCTTGGGCCATACGACC | |
| | CGGCAGACCCTATGGTGTTGGAGGTGTCAGTGGTGGGAAAAGATG | |
| | CNGTGTGGAGTTTATGGCAAGCCCCAGTGGGAGAATCACAACGCA | |
| | GGCCCCTGGGGTTCTGGAGCAAGGCTGCAAGTCTTTTGAAAAACA | |
| | GCTCTTNNNATGCTACTGGTAGAGANGGAACGCTTACCATGGGAC | |
| | ACCAAGTGACACGNANCAAAATGTACCCCGTCATGAGCTGGGTTC | |
| | TGTCGGACCCACCAAGTCATAAGGTCGGGCGGGCCCAGCAGCAAT | |
| | CCATCGTAAGATGGAAGTGGTACATCCGGGATCGAGCACGAGGAG | |
| | GACCACAAGTAAGCTGCATGAGCAGGTAGCCCAGACCCCCATGTC | |
| | ACCCACCACNGTTGCACCAGCGCTCCTTCAGCTCACACCTATGGCC | |
| | GTATGGAGNAGAGGTGTGNTCCTGATCGACCAGCTGAAGGAGGAG | |
| | GAAAAANGCCCGAGCTTGGTTCACGGATGGGTCGGCTCGGTATGT | |
| | GGGTGCAAGCCGAAAATGGACGGCGGCTTGCACTACAGCCNCACT | |
| | CAGGGGTGGCCTTGAAAGACAGTGGNGAGGGAAAATCTTCCCAAT | |
| | GGGCAGAGCTTCGGGCGGTGCACCTGGTCATCCACTTTGTGTGGA | |
| | AGGAGAAGTGGCCCGAGGTNAGAATATATACGGACTCATGGGCAG | |
| | TGGCGAATGGCTTGGCCGGCTGGTCAGGGGCCTGGAAGGAGAAA | |
| | GATTGGAAGATCGGAGACAAGGAGGTCTGGGGANTAGAGGCATG | |
| | TGGATGGACNTATGGGAGTGGGCACGAAGTGTGAAGATCTTTGTA | |
| | TCACATGTTAACGCCCACCAGAGAGCATCCACCACGGAAGAGGCA | |
| | CTAAACAACCAAGTAGACAAAATGACTCGGCCAGTTGACGTCAGC | |
| | CAGCCTCTGTCATCGGCCACCCCANGTGCTGGCACAATGGGCACA | |
| | TGAACGGAGTGGCCACGGTGGCAGAGATGGAGGCTACGCATGGGC | |
| | CCAACAGCATGGACTCCCACTCACCAAGGCTGATCTAGCTACTTGC | |
| | TGCCNCNNCTGAATGTCCAACCTGCCAGCAACAGAGACCGATCCC | |
| | CGATATGGCACCATTCCTCGAGGAGACCAACCAGCCACTTGGTGG | |
| | CAAGTTGACTACATTGGGCCCCTTCCATCCTGGAAGGGNCAGCGG | |
| | TTCATCCTNACAGGAATACATATTCCGGGTATGGGTTTGCCTTTCC | |
| | TGCCCGCAGGGCCTCAGCCAGCACCACTATCCGAGGGCTTACGG | |
| | AGTGCCTGATCCACNGGCATGGGATCCCACACAACATNGCATCNG | |
| | ACCAGGGACCCACTTTACAGCAAAGGAGGTGCGGGAGTGGGCCCA | |
| | TGACCATGGGATCCACTGGTCNTATCACATACCGCACCATCCAGA | |
| | AGCTGCCGGCCTGATAGAGCGNTGGAACGGCCTNCTGAAGGCACA | |
| | GCTGAAGCGCCAGCTCGGAGACGATACTCTGCGAGGATGGGGCGC | |
| | CATCCTTCAGGATGCAGTGTCCCCAATAGGAAGAATACATGGGTC | |
| | CGGGAACCAAGGGGTGGAAGCAGGAGTGTNGCCCCACTTACCATC | |
| | ACTCCCAGTGACCCACTGGGGGANTTTGTGCTTCCCGTCCCCGCA | |
| | ACTCTGGGCTCTGCAGGNTTAGAGGTCCTGGTCCCCAAAGGGGGN | |
| | ACGCTCTCGCCAGGGGACACAGCGTCCCATTGAACTNTANAGCTA | |
| | CGGCTGCCGCCTGGGCACTTTGGGCTCCTTGTGTCCAGGGACCAGC | |
| | AGGCAAGAAGAGGAGTCACCATCTTGGCAGGGGTAATTGACCCT | |
| | GATCATCAGGAGGAGGTAGGGCTGCTNTTACACAATGGGGGCAGG | |
| | GAGGAATACGTNTGGAACCCAGGTGATCCACTTGGGCGCCTCTTG | |
| | GTACTCCCTTGCCCAATTNTGACTGTAAATGGACAAGTGCAGCAAC | |
| | CCCGGCCTGAGAAGGGCATGGTNACCAGGGGCTCAGACCCCTCA | |
| | GGAATGAGGGTCTGGGTCACGCCACCAGGTAAGCCACCGAGACCA | |
| | GCAGAGTGNTAGCTGAGGGTGAGGGGAATCTAGAATGGATAGTGG | |
| | AGGAGGGAGACGATGAGTATCAGTTGCGGCCCCGAGACCAACTGA | |
| | GCGACGGGGGCTGTAGTTCGTCCCACTAACCTCCCTCTTCTAAGT | |
| | TTCCCTCAGGAAGAGAGGCCCACNGGAATCCTGGAGGAGCTGCTC | |
| | CTGAACCGAACGTGTATGGAGAAGTGGATCCGNGCGGCGCAAGGG | |
| | GTGGAC | |
| 15 | TGTGGCAGCCACGGAGGTGCGCCGCTCGGATCTCCCTTCAAGAAA | Consensus sequence |
| | GAACTTGCCGTTCAGCTGCGAGGAGTGCGGTTAGCTGACAGCCTC | for transposable |
| | CAGCTGTTAGCGCCTTCAGGATCCGCCTCAGCTTTCGAGCCGAGGC | elements that belong |
| | CACGCTCTTCCCGGGCAGCCCCCAGCCAATGACTGAGCANGGCG | to ERV3-16A3_LTR |
| | GGGGTACNAGGGCCTGGCCATTTCTGCCCAACGCGGGACTCCTCT | subfamily |
| | AACGGGCAATCTTTGCTCCGGAAGAGCTCCCCGTTGGGTTGGCCG | |
| | AGACTTTGTCAGATCTGCATCGCGGTCTGAGGCTCTCCCTGCCCAA | |
| | TCCTGCTTCCTCTCCTTTTNTCTTTCACAGGCGTTACCCCCCAA | |
| | TAAACCTCTTGCACTCCTAACTCCGTCTCAGCGTCTGCTTCCCGGA | |
| | GGACCCAACTGACACA | |
| 16 | GATTTTGGTACCGAGAGTGGTTCTAGAGGAACAGAATTTTAAGGA | Consensus sequence |
| | TGAGTTTTCTGAATTGGTTCTGGGGTTTCTGGAATTGGCTCTCTA | for transposable |
| | ATCTGATTAGATTTAAAGACGCTAATGACTCTATTTCCAGTAGTAA | elements that belong |
| | AGAGAGCACTGATAGTCCATGGCGTGATCTGGCAATAGAGATAC | to ERVL-B4 |
| | GCAAAATATCNCCATTGGATACTCCTAATCAACCACTTATAAGAA | subfamily |
| | GCAAGGANCTGGGTGACTNTGTATATGATACTTTCGAACATTTTT | |
| | GGNAAACTAACGAATATAATGAGATTGGCTGGTTGCTCCTAATGT | |
| | CGCTGGACAAAGTGGNGAAAGAAAAGGATGAGCTCAGGGATTCG | |
| | AATTCCCAGCTCAAGCGCCGCATAAATGACCTGAAAGCTTCTATGT | |
| | GTGCCCTGAAGGAGACCCTTATCTCCTGTAGCCGCAGGGCTGAGA | |
| | TTGCTGAAAATCAAACGCAGAATCTCATCCTGCGACTGGCTGAATT | |
| | ACAACGCAAGTTGAACTCCCAGCCTCGCAGGGTGTCTACTGTTA | |
| | AAGTGAGGGCATTGATTGGGAAAGAATGGGATCCTGNAAGTTGGA | |
| | ATGGGGACGTGTGGGAAGACCCTGATGAAGCTGGGGACATTGAGC | |
| | CCCTAAATTCTGATGAGTCTTCTTTGCCAGTGGAAGNGGCCTCCCC | |
| | ACCCCCAGTGGAAGCGGCCTCCCCACCCCCAGTGGTAGCGGCCT | |
| | CTCCACCCCCGTCTGAGGGGATTAACCCTGCATTGCCTGAGGAAAC | |
| | TGTAATGGCCTCCCCTGAGGCAGTTGCCATGCAAGACAATGCTG | |
| | ATTCTCCTCAGGACCCACCCCCACCACCCCTCTTTGCTTCTAGACC | |
| | TATAACTAGACTCAAGTCCCAGCAGGCCCCTAAAGGTGAGGTAC | |
| | AAAGTGTGACCCATGAGGAGGTGCGCTACACTCCAAAAGAACTAC | |
| | TTGAGTTTTCTAATTTATACAGACAGAAATCCGGGGAACATGTGT | |
| | GGGAATGGATATTAAGGGTGTGGGATAATGGTGGAAGGAACATAA | |
| | AGTTGGATCAGGCCGAATTTATTGATATGGGCCCACTAAGCAGAG | |
| | ATTCTGCATTTAATGTTGCAGCTCGGGGAGTTAGAAAGGGCTCTAA | |
| | CAGTTTGTTTGGTTGGTTGGCTGAAACATGGACCAAAAGGTGGC | |
| | CCACAGTGAGCGAATTGGAAATGCCGGACCTGCCTTGGTTTAATGT | |
| | AGAGGAAGGGATTCAAAGGCTTAGGGAGATTGGAATGTTAGAGT | |
| | GGATTTGTCATTTAAGACCTACTCACCCACACTGGGAGGGTCCAGA | |
| | AGACATACCTTTCACCANNACTGTGAGAAATAAATTTGTGAGGG | |
| | GAGCCCCAGCATCCTTGAAGAGCTCTGTGATCGCTCTTCTCTGTAG | |
| | GCCAGACCTTACAGTGGGAACTGCAGCCACTGAATTGGGAAACC | |
| | TAAATGCAATGGGAGTAATTGGATCCCGGGGTGGCAGGGGCCAAG | |
| | TGGCGGCACTCAACCGCCAAAGGCAAGGTGGGCGTGGTTACCGTA | |
| | ATGGACAGCAGAGTCAAAGCAGCAATCAGAATAGTCTGACTCGCG | |
| | CAGACCTATGGCGTTGGCTAGTTGATCATGGTGTTCCTAGAAGTG | |
| | AAATAGATAGGAAGCCTACTAAATTCTTACTTGATCTGTATAAGCA | |
| | GAAAAGTTCTAGGTCAAGTGAACAAAAGTCTAACTTGAATCATA | |
| | AAAACAGAGAGTCACGGCCCCTCAATCAATTCCCAGACTTGAGCC | |
| | AGTTTACAGACCCAGAACCCCTTGAATGAAGGGGAGGCCGGGTCC | |
| | CCTTGAGGAAGGACCCCGGTACACTGCCAAAAATTTATACTGTTA | |
| | ATCTTTCTCCCAGCCTTCCCCAAAGGGACCTACGGCCTTTTACCA | |
| | GGGTAACTGTGCATTGGGGAAAAGGAAATAATCAGACCTTTCGGG | |
| | GACTACTGGACACTGGCTCTGAACTGACACTAATTCCAGGAGACC | |
| | CAAAACGTCACTGTGGTCCACCAGTCAGAGTAGGGGCTTATGGAG | |
| | GTCAGGTGATCAATGGAGTTTTAGCTCAGGTCCGTCTCACAGTGG | |
| | GCCCAGTGGGTCCCCGAACCCATCCTGTGGTTATTTCCCCAGTTCC | |
| | GGAATGCATAATTGGAATAGACATACTCAGCAGCTGGCAGAATC | |
| | CCCACATTGGTTCCCTGACCTGTGGAGTGAGGGCTATTATGGTGGG | |
| | AAAGGCCAAGTGGAAGCCACTAGAACTGCCTCTACCTAGGAAAA | |
| | TAGTAAACCAAAAGCAATACCGCATTCCTGGAGGGATTGCAGAGA | |
| | TTAGTGCCACCATCAAGGACTTGAAAGATGCAGGGGTGGTGATTC | |
| | CCACCACATCCCCATTCAACTCGCCTATTTGGCCTGTGCAGAAGAC | |
| | AGATGGATCTTGGAGAATGACAGTGGATTATCGTAAGCTTAACC | |
| | AGGTGGTGACTCCAATTGCAGCTGCTGTACCAGATGTGGTTTCATT | |
| | GCTTGAGCAAATTAACACATCCCCTGGTACCTGGTATGCAGCTA | |
| | TTGATCTGGCAAATGCCTTTTTCTCCATACCTGTCAATAAGGNCCA | |
| | CCAGAAGCAGTTTGCTTTCAGCTGGCAAGGCCAGCAATACACCT | |
| | TCACTGTCCTACCTCAGGGGTATATCAACTCTCCAGCCCTATGTCA | |
| | TAATTTAGTTCGCAGGGATCTTGATCGCCTTTCCCTTCCACAAG | |
| | ATATCACACTGGTCCATTACATTGATGACATTATGCTGATTGGACC | |
| | TAGTGAGCAAGAAGTAGCAACTACTCTAGACTTATTGGTAAGAC | |
| | ATTTGCGTGTCAGAGGGTGGGAAATAAATCCGACAAAAATTCAGG | |
| | GGCCTTCTACCTCAGTGAAATTTCTAGGGGTCCAGTGGTGTGGGG | |
| | CATGTCGAGATATCCCTTCTAAGGTGAAGGATAAGTTGTTGCATCT | |
| | GGCCCCTCCTACAACCAAAAAAGAGGCACAATGCCTAGTGGGCC | |
| | TCTTTGGATTTTGGAGGCAACATATTCCTCATTTGGGTGTGTTACTC | |
| | CGGCCCATTTACCGAGTGACCCGAAAAGCTGCTAGTTTTGAGT | |
| | GGGGCCCAGAACAAGAGAAGGCTCTGCAACAGGTCCAGGCTGCTG | |
| | TGCAAGCTGCTCTGCCACTTGGGCCATATGATCCAGCAGATCCAA | |
| | TGGTGCTTGAAGTGTCAGTGGCAGATAGGGATGCTGTTTGGAGCCT | |
| | TTGGCAGGCCCCTATAGGTGAATCGCAGCGCAGGCCCTTAGGAT | |
| | TTTGGAGCAAAGCCCTGCCATCCTCTGCAGATAACTACTCTCCTTT | |
| | TGAGAAACAGCTCTTGGCCTGCTACTGGGCCTTAGTAGAGACTG | |
| | AACGCTTAACCATGGGCCACCAAGTTACCATGCGACCTGAGCTGC | |
| | CCATCATGAACTGGGTGTTATCTGACCCACCAAGCCATAAAGTTG | |
| | GGCGTGCACAGCAGCACTCCATCATCAAATGGAAGTGGTATATAC | |
| | GTGATCGGGCCCGAGCAGGCCCTGAAGGCACAAGTAAGTTACATG | |
| | AAGAAGTGGCCCAAATGCCCATGGTCCCCACTCCTGCTACACTGCC | |
| | TTCTCTCTCCCAGCCTGCACCTATGGCCTCATGGGGAGTTCCCT | |
| | ACGATCAGTTGACAGAGGAAGAGAAGACTCGGGCCTGGTTTACAG | |
| | ATGGTTCTGCACGATATGCAGGCACCACCCGAAAGTGGACAGCTG | |
| | CAGCACTACAGCCCCTTTCTGGGACATCCCTGAAGGACAGTGGTG | |
| | AAGGGAAATCCTCCCAGTGGGCAGAACTTCGAGCAGTGCACCTGG | |
| | TTGTTCACTTTGCTTGGAAGGAGAAATGGCCAGACGTGCGATTATA | |
| | TACCGATTCATGGGCTGTGGCCAATGGTTTGGCTGGATGGTCAG | |
| | GGACTTGGAAGGAACATGATTGGAAAATTGGTGACAAGGAAATTT | |
| | GGGGAAGAGGTATGTGGATAGACCTCTCTGAATGGGCAAAAAACG | |
| | TGAAGATATTTGTGTCCCATGTGAATGCTCACCAAAGGGTGACCTC | |
| | AGCAGAGGAGGATTTTAATAATCAAGTGGATAGGATGACCCGTT | |
| | CTGTGGATACCAGTCAGCCTCTTTCCCCAGCCACCCCTGTCATCGC | |
| | CCAATGGGCTCATGAACAAAGTGGCCATGGTGGCAGGGATGGAG | |
| | GTTATGCATGGGCTCAGCAACATGGACTTCCACTCACCAAGGCCG | |
| | ACCTGGCTACGGCCACCGCTGAGTGCCCAATCTGCCAGCAGCAGA | |
| | GACCAACACTGAGTCCCCGATATGGCACCATTCCCCGGGGTGATC | |
| | AGCCAGCTACCTGGTGGCAGGTTGATTACATTGGACCGCTTCCAT | |
| | CATGGAAGGGGCAGCGTTTTGTTCTTACTGGAATAGACACTTACTC | |
| | TGGATACGGATTTGCCTTCCCTGCACGCAATGCTTCTGCCAAAA | |
| | CTACCATCCGTGGACTTACAGAATGCCTTATCCACCGTCATGGTAT | |
| | TCCACACAGCATTGCTTCTGATCAAGGAACTCACTTCACAGCAA | |
| | ANGAAGTGCGGCAATGGGCCCATGCTCATGGAATTCACTGGTCTT | |
| | ACCATGTTCCCCACCATCCTGAAGCAGCTGGCTTGATAGAACGGT | |
| | GGAATGGCCTTTTGAAGACTCAGTTACAGCGCCAGCTAGGTGGCA | |
| | ATACCTTGCAGGGCTGGGGCAAGGTTCTCCAGAAGGCTGTATATG | |
| | CTCTGAATCAGCGTCCAATATATGGTGCTGTTTCTCCCATAGCCAG | |
| | GATTCACGGGTCCAGGAATCAAGGGGTGGAAATGGGAGTGGCAC | |
| | CACTCACTATTACCCCTAGTGACCCACTAGCAAAATTTTTGCTTCC | |
| | TGTTCCCGCGACCTTATGCTCTGCTGGCCTAGAGGTCTTAGTTC | |
| | CAAAGGGAGGAATGCTTCCACCAGGAGACACAACAATGATTCCAT | |
| | TGAACTGGAAGTTAAGACTGCCACCCGGCCACTTTGGGCTCCTCA | |
| | TGCCTCTGAATCAACAGGCAAAGAAGGGAGTTACTGTGCTGGCTG | |
| | GGGTGATTGATCCTGACTACCAAGGGGAAATTGGACTGCTACTCC | |
| | ACAATGGAGGTAAGGAAGAGTATGTCTGGAATACAGGAGATCCCT | |
| | TAGGGCGTCTCTTAGTATTACCATGCCCTGTGATTAAGGTCAATG | |
| | GAAAACTACAACAACCCAATCCAGGCAGGACTACTAATGGCCCAG | |
| | ACCCTTCAGGAATGAAGGTTTGGGTCACCCCACCAGGTAAAGAAC | |
| | CACGACCAGCTGAGGTGCTTGCTGAAGGCAAAGGGAATACGGAAT | |
| | GGGTAGTGGAAGAAGGTAGTTATAAATACCAGCTACGACCACGTG | |
| | ACCAGTTACAGAAACGAGGACTGTAATTGTCATGAGTATTTCCTCC | |
| | TTATTTTGTTATGAATATGTTTGTGTGTATATATACATATATTA | |
| | AGCAAATATCTTTGTTTTCTTTCCTCTCTTATTCCCTTATCATGTAA | |
| | CATAAGATGTATTGACTTTATATCATAGTATTTAAGTATTGTT | |
| | AATTTTACATCATAGTATTTAAGTTACGGGATATCAAGGAGAAGA | |
| | GTAAACATCACTCAAGGACTTTACCTCCTCTTCTGGGGAAGGGGT | |
| | TAGTGCGTTTTCGGTTGTACGCAGGATAGTTGTATCATGTTAGGCG | |
| | GAATTATGACCTTGTTATTGTCTTTATTTGGAGATTAAGTATGG | |
| | TTTAAGGAGATGCGTATGGGTGCCAAGTTGACAAGGGGTGGACT | |
| 17 | ATTATTGGTACCGGGAGTGGTTCCAGGGGAACAGAACCTTAAGGA | Consensus sequence |
| | TGGGAATCTGGAATTGGTTCTCTGATCTGATTAGATTTAAAGGCG | for transposable |
| | CTAATGACCCTGTTTCCAGTGGTAAAGGGGACACTGGTAGTCCATG | elements that belong |
| | GCATGCAGTGGCAAAAAGTTACTCAAATTATCACCTGTGGNCAC | to ERVL-E |
| | CTGTAATCAAGTGCCTATAGAAGGCAAGGCTTTGGGTGACCANGT | subfamily |
| | ANTTGCTGCCNTAGAACATTTTAGTGGAAATAAGGAGTATAATGA | |
| | NGTTGGTTGGTTGCTTCTAANTGCGCTGGAGAACTTGGAGAAAGA | |
| | AAATGATGAGCTCAGGGCTTTAAATTCCCAGCTCAAGNTCCGGGT | |
| | AAGGGACCNGAAAGCTTCTATGACTGCCCTGAAAGAAACCCTTAT | |
| | CTCCTGTAGCCGCAGGGCTGAGATTTCTGAAAACCAAACCCAAAG | |
| | TCTNATCCTGCGGGTGGCTGAATTACAACGCAAATTGAATTCNCAA | |
| | CCTCGCAGGGTNTCTTNTGTTAAAGTTAGGGCATTGATTGGGAA | |
| | GGAATGGGACCCTGAAAATTGGAATGGGGACATNTGGGCGGATTC | |
| | CGATGAAGCTGGGGACCTTGAACCCCTAAATTCTGCCGAGCCTTC | |
| | TTTGCCAGTAGAAGCAGCCCTTTCNCCCCTGTCTGAGGAGGTTAGT | |
| | CTCCCCTTGCCTGAAGAANCTGTAATGGCCTCCCCTGAGGTAGT | |
| | TGCCTTGCAAGGNANTGCTGATTCTCCTCAGGACCTACCCCCACCA | |
| | CCTCTCNTTGCTTCTAGACCTATAACTAGACTCAAGTCCCAGCA | |
| | GGCCCCNAGGGGTNAGGTACAAAGTGTGACCCATGAGGAGGTAN | |
| | NNTACACACCAAAAGAATTGCAAGATTTTNCCAATTTATATCGAC | |
| | AGAAACCTGGGGAATATGTGTGGGAATGGATNCTAAGGGTGTTGG | |
| | ATCANGGTGGAAGGAATATAACGTTGGATCGGGCCGAATTTATTG | |
| | ATATGGGTNCACTAAGCAGAGATTCTGGATTCAATGTGTTAGCTCG | |
| | AGNAGCTGGAAGTGGCTCTAACAGTTTGCTTGGTTGGTTGACTGA | |
| | AACNTGGACTCAAAGGTGGCCTACANTNAATGAAGTTGAGATGCC | |
| | AGAACTTCCTTGGTATANTGTAGAGGAAGGNATCCAAAGGCTTAG | |
| | GGAGATNGGAATGTTGGAGTGGATTTATCATGTAAGACCTGCTCA | |
| | CCTACACCCTCTAACTATGTCCCCGGGAGGGTCCAGGGACACTCC | |
| | CTTCACCAAGGCNTTGAGAAATACATTNGTGAGGGGAGCACCAGC | |
| | ATCCTTGAAGAGCTCTGTGGTGGCTNTTCTCTGTAGGCCAGGNAT | |
| | GACGGTGGGAGATGCTGCCATTGAAATGGGCTCCCTGANTTCAAT | |
| | GGGGATGATGGGATCCCGGGGTGGCAGAGGCCAAGTGGCGGCACT | |
| | TAACCGCCAGAGACAAGGTGGGCGCGGTTACCGTAATGGGCAGCA | |
| | GAGCCAAAGCGGTAATCAGAATGGTTTGACCCGCAGAGATCTTTG | |
| | GCGNTGGCTAATTGATCATGGTGTCCCTAGGANTGAAATAGATGG | |
| | GCAGCCTACTAAAGTCTTACTTGATTTGTATAAGCAGAAAAGCTC | |
| | TAGGTCTGGTGAACAGAAGTCTGACTTGAGTCACCAACGGAGAGT | |
| | CACGGCCCCTCANCCAGTTCCCAGACTTGAGCCAGTTCACAGCCA | |
| | GACCCCTTGAATGAAGGGGAGGCCGGGTCCCCTTGAGGAAGGACC | |
| | CTGCNACACTGCCAAAAATNTATACTGTAAATCTTCCTCCNAGCC | |
| | TTCCCCAAAGGGACCTGCGGCCATTTACCAGGGTGACTGTGCACTG | |
| | GGGAAAGGGAAATANCCAGACTTTTNAGGGATTACTGGACACTG | |
| | GCTCTGAACTGACGCTAATTCCTGGAGACCCAAAACGCCACTGTG | |
| | GTCCACCAGTCAGAGTAGGGGCTTATGGAGGTCAGGTGATNAATG | |
| | GAGTTTTGGCTCGAGTCCGTCTCACAGTGGGCCCAGTGGGTCCNCG | |
| | AACCCACCCTGTGGTTATTTCCCCAGTTCCNGAATGCATAGTTG | |
| | GAATAGACATACTCAGCAACTGGCAGAATCCCCACATTGGTTCCCT | |
| | GACCCGTGGAGTGAGGGCTATTATGGTAGGAAAGGCCAAGTGGA | |
| | AGCCNCTGGAACTGCCTCTNCCTACCAAAATAGTAAACCAAAAGC | |
| | AATACCGCATCCCTGGAGGAATTGCAGAGATTAGTGCCACCATCA | |
| | AAGACTTGAAAGATGCAGGGGTGGTGATTCCTACCACATCCCCAT | |
| | TTAACTCGCCTGTTTGGCCTGTGCAGAAGACAGATGGATCTTGGA | |
| | GAATGACAGTGGATTATCGTAAACTTAATCAGGTGGTGACTCCAA | |
| | TTGCAGCTGCTGTTCCAGATGTGGTNTCTTTACTGGAGCAAATCA | |
| | ACACANCCCCTGGCACCTGGTATGCAGCTATTGATCTGGCAAATGC | |
| | TTTTTTCTCTATACCTGTTAGTAAAGACCACCAGAAGCAGTTTG | |
| | CTTTCACCTGGCAGGGNCAGCAGTACACCTTCACTGTCTTGCCTCA | |
| | GGGCTATGTCAACTCTCCNGCTCTCTGTCATAATNTAGTCCGCA | |
| | GGGACCTTGATCGTCTTNNCATTCCACAGGACATCACGCTGGTCCA | |
| | CTACATTGATGACATCATGCTGATTGGACCTGGTGAGCAGGAAG | |
| | TAGCAAGTACTCTAGACGCCTTGGTAAGACACATGCGTGCCAGAG | |
| | GGTGGGAGATAAATCCCACGAAAATTCAGGGGCCTGCCACCTCGG | |
| | TGAAGTTTCTAGGGGTCCAGTGGTCTGGGGCATGTCGAGATATCCC | |
| | TTCCAAGGTGAAGGACAAGTTGCTGCATCTNGCNCCTCCTACCA | |
| | CTAAGAAAGAGGCACAATGCTTGGTGGGCCTCTTTGGATTTTGGA | |
| | GGCAACATATACCNCATTTGGGCGTGCTGCTCCGACCCATTTACC | |
| | GAGTAACCCGNAAGGCTGCCAGTTTTGAGTGGGGCCCAGAGCAAG | |
| | AGAAGGCTCTGCAGCAGGTCCAGGCTGCNGTGCAAGCTGCTCTGC | |
| | CACTTGGGCCNTATGACCCAGCAGATCCAATGGTGCTCGAAGTGT | |
| | CTGTGGCAGATAGGGATGCTGTATGGAGCCTCTGGCAAGCCCCNA | |
| | TAGGNGAATCACAGCGCAGACCCCTAGGATTTTGGAGCAAAGCCA | |
| | TGCCATCTTCTGCAGATAACTATTCTCCTTTTGAGAAACAGCTCC | |
| | TGGCTTGCTACTGGGCCCTGGTAGAGACTGAACGCCTGACCATGG | |
| | GNCACCAAGTNACCATGCGACCTGAGCTGCCCATCATGAACTGGG | |
| | TGTTATCTGACCCACCAAGCCATAAAGTTGGGCGTGCACAGCAGC | |
| | ANTCCATCATCAAGTGGAAGTGGTATATACGAGATCGGGCTCGAG | |
| | CAGGTCCNGAAGGCACAAGTAAGTTGCATGAGCAGGTGGCTCAGA | |
| | CTCCCATGGCNCCTACTCCTGCTGCATTGCCTCCTCTCCCTCAAC | |
| | CCGCACCTATGGCCTCATGGGGAGTTCCCTATGACCAGTTGACTGA | |
| | GGAAGAAAAAACTCGGGCCTGGTTTACAGATGGTTCTGCACGAT | |
| | ATGCTGGCACCANCCGAAAGTGGACGGCTGCAGCACTACAGCCCC | |
| | ACTCAGGGGTGGCCCTGAAGGACAGTGGTGAAGGGAAATCCTCCC | |
| | AGTGGGCAGAACTTCGAGCAGTGCACCTGGTTGTCCACTTTGCCTG | |
| | GAAGGAGAGATGGCCAGAGGTACGGATCTACACTGATTCATGGG | |
| | CAGTGGCTAACGGTTTGGCTGGATGGTCAGGGACTTGGAAGGAAC | |
| | ANGATTGGAAGATTGGTGACAAGGAGGTCTGGGGAAGAGGTATGT | |
| | GGATGGACCTCTCGGAATGGGCACAGAGTGTGAAGATATTTGTGT | |
| | CCCATGTGAATGCTCACCAAAGGGCANCCNCNGCAGAGGAGGNTC | |
| | TCAATAATCAGGTGGACAAGATGACCCGTTCTGTGGATGTCAGTC | |
| | AGCCTCTTTCCCCAGCCACCCCNGTGCTTGCTCAATGGGCTCATG | |
| | AACAAAGTGGCCATGGTGGCAGGGATGGAGGCTATGCATGGGCTC | |
| | AGCAACATGGACTTCCNCTCACCAAGGCTGATCTGGCTACNGCCA | |
| | CTGCTGAGTGCCCAACCTGCCAACAGCAGAGACCAACGCTGAGCC | |
| | CCCGATATGGCACCATTCCCCGGGGGGACCAGCCAGCCACCTGGT | |
| | GGCAGGTTGATTACATTGGACCNCTTCCATCATGGAAGGGGCAGC | |
| | GATTTGTCCTCACTGGAATAGACACTTATTCTGGATATGGATTTG | |
| | CCTTCCCTGCCCGCAATGCTTCTGCCAGNACCACCATCCGTGGACT | |
| | TACAGAATGCCTTATTCACCGTCATGGTATTCCACACAGCATTG | |
| | CTTCTGACCAAGGAACTCATTTTACAGCAAANGAAGTGCGGCAAT | |
| | GGGCTCATGCCCATGGAATTCACTGGTCTTACCACGTNCCCCATC | |
| | ACCCNGAAGCAGCTGGCCTGATAGAACGGTGGAATGGCCTNTTGA | |
| | AGACTCAGTTACGGCGCCAGCTGGGNGACAACACCTTGNAGGGCT | |
| | GGGGTNNTGTCCTNCAGGATGCGGTATATGCTCTGAATCAGCGAC | |
| | CAATATATGGTGCTGTTTCTCCCATAGCCAGAATNCACGGGTCCG | |
| | GGAATCAAGGGGTGGAAGTGGGAGTGGCTCCTCTCACTATTACNC | |
| | CTAATGACCCACTNGCAAAATTTTTGCTTCCCGTCCCCGCAACTT | |
| | TGGGCTCTGCTGGTTTAGAGGTCTTAGTTCCCAAGGGAGGAATGCT | |
| | TCCACCAGGGGACACAACAATGGTTCCATTGAACTGGAAGCTGA | |
| | GACTGCCACCTGGCCACTTTGGGCTCCTCATGCCACTGAACCAACA | |
| | GGCAAAGAAGGGAGTTACTGTACTGGCTGGGGTGATTGATCCTG | |
| | ATTATCAAGGGGAAATTGGGTTGCTGCTACACAATGGGGGCAAGG | |
| | AGGANTATGTCTGGAATNCAGGAGATCCTCTGGGGCGCCTCTTAG | |
| | TACTCCCATGTCCNGTGATAAAAGTTAATGGAAAACTACAGCAAC | |
| | CCAATANAGGCAGGACCGCTAANGGCTCAGACCCTTCAGGAATGA | |
| | AGGTTTGGGTCACCCCACCAGGCAAAGAACCACGACCAGCTGAGG | |
| | TGCTNGCTGAGGGCAAAGGGAATATGGAATGGGTAGTGGAAGAA | |
| | GGAAGTTATAAATACCAGCTACGACCNCGTGACCAGTTGCAGAAA | |
| | CGAGGACTGTAGTAGTTATGNGTATTTCTTCCTTGCTTTGATATGA | |
| | ATATATTTGTGATATATATATTAACNAATATCTTTNTTTTCTTTCCT | |
| | CTCTCATTCCCCTACTATCTAACATAAGATGTGTTAATAGTAG | |
| | TTAACCTTATATCTCAGTATTTAAGTTACAGGATATCAAAGGGGGA | |
| | NTGTGACTCAGCTAGAAGAGNAATGAACATCACCCAGAGATGGA | |
| | TAAAGTGACNTNTGGGACTTTGTATCCTCTTTTGGGGAGAGGGTTA | |
| | GCGTGTTTTCGGTTGTACGAGGGATAGTTGCATCATGTTAGGCG | |
| | GAAGCATGATTTTGCTATTGTCTTTATTTGGAAGTTAAATATGGTT | |
| | NAAAGAGGTGTGTATGGATGCCGAGTTGACAAGGGGTGGAC | |
| 18 | GTTGGTACCAGGAGTGGTCCGAGAAAGCAGACGNTNCTAAGATGG | Consensus sequence |
| | GATTTTGGAGCTGGATCACCCGCCGNCCGGCTGGCAATGAGGACC | for transposable |
| | CCATCACTGGTGGTAGGTGGAGCACGGATAGCCCCTGGCACGAGG | elements that belong |
| | TAGCGGTGCAATTGTTAAAACTTTCACCGGTGGTGAACTGGGATG | to HERV16 |
| | GNATACCGGTGGAAAGAGAATGCACTGGCNGGTGCAATGTNTCAG | subfamily |
| | GCGTTTGAGAAATATGGGANGAATTAANTACATGNAAGGACAATG | |
| | GAATTGGATGGCTGTTGCTAAGCNCGACTGACGCTCTGGAAAAAG | |
| | ACAATGAAAGGCTGAGAGCGATTAATCGNCAATTNAAAGCTAAGT | |
| | GTGAAAGCCAGAGGGCCTCCTTGGCAGCATATAAAGAGACTCTCA | |
| | TCTCCTGCAGCCGGAGGGCAGAGAAAGCTGAGGATCAGGCCCAGG | |
| | ACTTAATCAGAGTAGCAGAGCTCCAGAGAAGGTTGAATTCTCAAC | |
| | CNAGGCAGGTCTGCTATGCCAAGGTCAGGGCCCTGGTTGGGAAAG | |
| | AATGGGACCCTGANACNTGGGATGGGGACATCTGGGTCGATGCNC | |
| | CTGAAAATCTTGAATCCCCAGATTCCCCTGAACCCTCTGGGCCTG | |
| | CAGAAGTGGCCCACTCCTCCCTGTTAAGGGCTAGCACTCNCTCCTT | |
| | GCTTCGCGNGAAGACGATGCAGAGGCCTCTNCCTTNGCAAGACA | |
| | ACATGCGCCCCCCTCAGGATCTGCCCCCACCTCCCCTCCTGGCCAC | |
| | CAGACCAATAACTAGGGTTAAGTCACAGCATAACCCGGCCGGGG | |
| | AAGTGCTGGGCCTGNTAAGGGAGGAAAGGGACTATACCCCAAAG | |
| | GAGCTGCAGGACNCGNCTAGCCAGCATGTACCGGCAGGACCGGGA | |
| | GAGTACGCATGGGACTGGATTCTGAGGGTGCTGGATCAAGGGGGN | |
| | CGGAACATAAAGTTGGATAAGGGAGAGTTTATCGATNTGGGAGCA | |
| | CTCTCCCGNGATACAGGATTTAACACCCTGGCAAGGACCCCGGGA | |
| | GATGGTGCNAACACGCTGCTAGGATGGCTCCTGGAAGCNTGGAGA | |
| | AAGCGATGGCCCACACTAAGTGAAGTAGAAATGCCAGAACTGCCG | |
| | TGGCAGACGGTGGAAGAAGGGATCAAAAGGCTCAGAGAAGTGGG | |
| | CATGCTAGAGTGGATATACTATGTAAGGCCGGAAAACCCACCAGA | |
| | TGACTATGTTCCGCGGGAGGGCCCAGAGGACACNCCATTTACCAA | |
| | AGCNATAAGGAATGCGCTGGTGAGAGGGGCACCAGCATCACTGAG | |
| | AAGCTCAGTGGTGGCTNTCCTCTGCAGGCCAGGGCTGACGGTAGG | |
| | AGANGCCGTTACAGAACTGGGCTCNCTGATAGCAATGGGGATGAT | |
| | AGGACCCCGAAATAATAGAGGCCAGGTGGCGGCGCTTAACCGTCA | |
| | GAAGCAAGGTGGGCGCAATTATCGTAATGNACGGCAAGGTCGGAG | |
| | TGGCAGCCGGGGGGGCCTGACCCGCAGAGAGCTATGGAGATGGTT | |
| | AATAGAACACGGCGTCCCTAGGGGCAAGATAGATGGGCAGCCAAC | |
| | AGGAAGNCATGGACAGTTAATAGAACATGGCGTCCTAGGGGCAAG | |
| | ATAGATGGGCAGCCAACAAGGGTGTATTGCTCAACTNAAACAANC | |
| | AAAAGAAATCAAGGATGGATGANCAGGAGGCTGAGGGCAGTCGC | |
| | CCCAATAAAAAGTCACGATCCCTTGCCCAGTTTCCGGACCTGAGCC | |
| | AGTTTTCAGACCCGGAACCCATTGACTGAAGGAGAGGCCGGGTCC | |
| | CCAGGAGGAAGGACCCTGCAACACCACGGCAAGTGTACACGGTAA | |
| | TGATTCCCCCAGTCCTTCCCCAAAGGGACCTACGGCCATTTACTCT | |
| | GGGTAACCGTACACTGGGGAAAGGGGAATACCCAGACATTTCGAG | |
| | GACTGTTGGACACAGGGTCCGAGTTGACATTGATACCCGGAGACC | |
| | CGAAGCGTCATCATGGCCCTCCCGTTAGAGTGGGGGCATATGGGG | |
| | GCCAGGTAATAAATGGAGTCCTGGCCCAGGTCCGGCTCACAGTGG | |
| | GTCCACTGGGTCCACGGACCCACAGTGGTCATTTCCCCGGTCCCCG | |
| | AATGTATAATTGGAATGGACATACTTGGTAGTTGGCANAACCCCC | |
| | ACATTGGTTCCTTGGCCTGTGGGGTAAGAGCTATCATAGTGGGGA | |
| | AGGCCAAGTGGAAGCCTCTGAAACTGCCCTCACCTTCTCCGGNCA | |
| | AGATAGTAAATCAAAAACAATATCGCATCCTCGGGGTGGAGAATG | |
| | GCAGAGATTAGTGCCACCCTTAAAGACCTAAAGGATGCAGGAGNT | |
| | GTGGTCCCCATCATATCTCCATTTAATTCACCAGTNCNGGCCCCTG | |
| | CAAAAACCAGACGGATCCTGGANGACGACAGTGGACTACCGCAA | |
| | ACTCAACCAAGTAGTANCCCAATTGCAGCTGCTGTGCCAGATGTG | |
| | GTATCTTTGCTAGAGCAGATTAACACGGCCTCAGGTACGTGGTAT | |
| | GCGGCCATTGATCTGGCGAATGCGTTCTTTTCCATCCCTATCAGAA | |
| | AGGAGGATCAGAAGCAGTTCGCATTCACNTGGAACGGACAACAG | |
| | TATACATTTACAGTCTTGCCCCAGGGCTNTGTTAACTCTCCTGCCCT | |
| | CTGTCATAATATAGTCCGAAGGGACCTGGACCATCTGGACATT | |
| | CCGCAGAACATCACATTGGTCCACTATATCGATGACATCATGCTAA | |
| | TCGGACCGGAATGAGCAAGAAGTGGCAAGTACGTTGGAGGCCTT | |
| | GGTAAGACACATGCGCTCCAGAGGGTGGGAGATAAACCCTACGAA | |
| | GATTCAGGGGCCTGCCACATCAGTGAAGTTTTTAGGGGTCCAGTG | |
| | GTCTGGGGCATGCCGGGACATCCCCCCAAAGTAAAGGACAAATTA | |
| | TTGCATCTTGCACCTCCCACCACNAAGAAGGAAGCACAACGCCTG | |
| | GTAGGCCTCTTCGGGTTCTGGAGGCAGCATATTCCACACTTGGGAA | |
| | TACTGCTCCGACCCATNTACCGGGTGACGCGAAAGGCTGCCAGC | |
| | TTTGAGTGGGGCCCAAGCAGGAAAGGGCTCTGTAGCTNACCGCAT | |
| | ATCGTGTNCGAANCGCANANNCGGNCCTCCAAGTCATAAAGTNGG | |
| | GCGGGCCCAGCAGCAGTCCATCATAAGGTGGAAATAGNATATCCG | |
| | AGATTAAGCCCGAGNAGGACCAGAGGGCGCAGGTNAGNTNCATG | |
| | AGCAGGTAGCCCAGACCCCCATGNCACCCACCACNGTTGCACCAG | |
| | CGCCTCTCCTTCGGCTCGCACCTATGGCCATATNGAGGNNTGTGGT | |
| | CCCGTATGACCAGCTGAAGGAAAGCTCGAGCTTGGTTTACAGGAT | |
| | CAGCTCGGTATGTGGGCGCAAGCCAAAATANGTGGTGGCTGCACT | |
| | ACAGCCCCATTCAGGGGTGGCCCTGAAAGGCAGCAGGGAGTGAAA | |
| | ATATCTTCCCAGTGGGCAGAGCTGCGAGCAGTGCACCTGGTCATC | |
| | CACTTTNCGTGGAAGGAGAAGTGGCCCGAGATGGTAATATATACG | |
| | GATTNCTGGACAGTGGCCAATGGCCTGGTCGGCTGGTCAGGAGCT | |
| | TGGAAGGAAAAGATTAGAGANAGGGAAGTCTGGGGTAGAGGCAT | |
| | GTGGATAGACATATNGGAAGTGGCACGAAATACGAAGATTTTTGT | |
| | ATCGNTNNTATGTTAACGCTCACANGAAAGCATCNACCATGAAAG | |
| | AGNCACTGAACAACCAAGTAGACAAAGTGACTTCGACCACTGACG | |
| | TTAGCCAGCCTTCGTCCCGGCCTCCTCAGANCTGGTNCAACGGACA | |
| | TCTCAANAAAGTAGCCATGGTGGCAGAGATGGAGGCTACGCATGG | |
| | GCCCAACAGCATGGACTCCTATNCACCAAGNCGATCTAGCTGCTG | |
| | CCCCGTNTGAATGTCCAACCTGCAGCAACAGAGACCAATGCTCTA | |
| | CCTCNAATACGGCACTATTTATGGAGACCACTTAGCGGCGAATTG | |
| | ACTATATTGGACGCCTTCCATCCTGGAANGGNCAGNGATTCATTC | |
| | TCACAGGAATANATACNTATTCCGGGTATGGGTTTGCCTTTCCTGC | |
| | CTGCANAGCCTCATCNAGCATCTGAGGGNCTTAGGAGTGCCTGA | |
| | TCCACAGGCATGGAATCCCACACAACATAGCATCTGANCAGGGCG | |
| | CTCACTTCACAGCAAAGAAGNGGCACAAGTGGGTCCANAATAACG | |
| | GGATCCACTTATCNTATCACGTACCGCACCATCCAGAAGNAGCTG | |
| | ACCTNATAGAACGCTTCTGAAGGCACAGNTGAAGCACCAGTNGAG | |
| | GCGAACTCTGAAAGAATTGGGTGCCATCCTCCAGGATGCAGTATA | |
| | TACATTGAATCANAGGCCTATNCGTNGGGCNTTGNNTCTAATNGG | |
| | GAGAATGGGATTAGGAATAAGAGGTGAAAGCAGGAGTGTGCCTA | |
| | CTCCCCATCGTAGTGGCCCACAAGGATCACAGCAGTGGTTTGAAC | |
| | ACGGATTGGACTACTGATATCCAGGAATCGCCGCACAAGAAGGNG | |
| | AATCCACATTTGGCAGGAGTAATTGACCNTGATCANCAGGAGGGG | |
| | TAGGGCTGCTNTCGCANAATGAGGAAGAAAGGAATATGTNTGGAA | |
| | CCAGGTGATCCNCTTGGGTGCCTCCTGGTACTCCCTTGCCCCATTT | |
| | AACTGTAAATGGACAATNCCACCGAAAAGAGCTCGGACCNCTCCG | |
| | GAATGAAGGTTTGGNTCACATCGCCNGGTAAGCCACCAAGACCTG | |
| | TCGAAGTGATAGCTGNGTGAGGGANATCTAGAATGGATAGTAGAA | |
| | GAGGGAGACGATGAGTACCAGTTNCGGCCCCGAGACCAACTGCAG | |
| | CAANNGGGGCTGTAGTTCGTCCCACTAACCTCCCTCTTCTAAGTTT | |
| | CGCTTCAGAAAGAGAAGCTTAAAGGAATAATGGAGGAACTGCTC | |
| | CNNGAACCTGNGTGGAGAAGTAGATCCGTCGAGTACAAAGGTGGA | |
| | C | |
| 19 | TTTCTGGCGAGCCAGCCAGGAGTGGAGACGACAGGTTTGCTGTCT | Consensus sequence |
| | CCTTTGCCTGTGGGNCTGGAGCCCCGGGCCGGGGGAGACCTGTGA | for transposable |
| | CCCCAGGCGCCGCCTGGGAGAACTTCAGCCCGGAGGGGAGATCGG | elements that belong |
| | CTCTCCCGTGACCCGGCGCCCCTNCCCGGCAGCGCAACGGAACCT | to HERV3 |
| | AAGGGGCTACAGGACGATTCCAGGAACAGCGCGCTNCAGGACCGC | subfamily |
| | GGTAAGGTTTGGGGCCCAAGGCAGGACCCGTCCCATAAGGACGGA | |
| | AGGGGAGCCTGATCACCTCCCGGGGTGTANCTAGTAGTCCGACCC | |
| | AGGAGCTGGGGGCGGCGAGAGTGGCTCGCCAATTCGGATGAAACT | |
| | TACACCCCAACCAAGAANAGGAACTGGGAGTGGGGAAGTGTGTG | |
| | AATGCGTGTGAAAGAGGCGGTTCCAGGAGGAGCCAATGCGGGGA | |
| | GTGACGTGTGGGGCCGCAGGTCTCTTAGCGTGGACCGTACGCTCC | |
| | GAGCGAAGTGTGGGGCCGACCGGGACTAGTGGCGAACGTCCTCCG | |
| | NNNCCGCCGCATACGGCTNAGGGAGGCGCCCCACAACTCAGTGAT | |
| | TGTGGTGGTCCGGGTTCGGGGNTNATACGAACCCTCCANTAAAGC | |
| | TAAGCGGCGTCTGAAAACTCCCGTAAGGGAGACGGTCTAATCGGT | |
| | CTGAAGCGAAAGTAAAAGAGTGAGTGTGTTGCGCCGTAACTGGGA | |
| | GGAAATGGGAGGGAAGTCGTCAAAACCCACCCCATTGGAATGCAT | |
| | GTTAAAGAACTTTAAGAAAGGTTNTGCAGGGGATTATGGAGTCAA | |
| | GTTGACCCCCCAGAGGTTGAGAACTCTCTGTGAATTAGAATGGCCC | |
| | TCTTTTGGTGTCGGATGGCCGGCCGAAGGAACTATAGATAGGGAA | |
| | ACAATTGGCCGTGTATTTAAGGTGGTGACNGGGGTCGGAGGACAG | |
| | CCAGGGCACCCAGACCAATTTCCTTATATTGACTCATGGCTAAATA | |
| | TAGTNCAGACNCGACCAGCATGGNTNCAGCCCTGCCTAGCGGCTT | |
| | ATTGCAAAACGCTCGTGGCTCGAGCCGAGCCNAAAGTGAAAGAAA | |
| | AATCAGCTTCGCCGGCAGCCACGGAGNTAAAGGGAAAGCCACAG | |
| | GAAGGCAAGAGAAGCCAGTTTTGCAGGAGCCGCCAGAGGAAACA | |
| | GAAATTCCTCCTCCNTATGTCCCAGCCTACCCCCCTTTACCGAGGC | |
| | CAACGGCCCCCGAGGAGCCAGATTCAGGNGNTAACACGCCCCAGG | |
| | TCTCACCCCGAAGGGAAGGATCGGAGCCTCGGGAGGCCAAGGAG | |
| | GGAAGTCAAGATAGTCAAGCGGGCCGCCTCNGATCTGGCCGCGCC | |
| | CGAGCTATGCAAATGCCTCTCAGGGAGACGCGAGGACCCGTCTAT | |
| | TATGATGANCAGGGCCANGTCCAAGGGGGGCAACGGACTTTCATC | |
| | TACCAGCCCTTTTCAACCACTGATCTCCTAAACTGGAAACACCATA | |
| | CTCCCTCCTACACGGAGAAGCCCCAGGCCCTCATAGATCTGATGCA | |
| | GTCCATCTTTCTGACACACAATCCAACCTGGCCAGACTGCAAGCAG | |
| | CTCCTCCTGACGCTGTTTAACACCGAGGAGCGCCGGAGGGTGAC | |
| | CCAGGCAGCCCTCCGCTGGCTAGAAGCCAATGCGCCAGAAGGCGC | |
| | ANTTAATGCCCAGGCATACGCTCAGGGCCAGTTCCCAGAAGCAGA | |
| | CCCCNACTGGGACCCAGATGATGCAACCCAGCTTCAGCGTCTGCA | |
| | GAGGTACCGAGAGGCACTCCTGCAAGGGCTAAGAGANGGCGGAA | |
| | AAAAGGCAATCAATATAGGAAAGATCTCAGAGGTGCTTCAGGGAG | |
| | CTGACGAGAGCCCNAGCCAGTTTTATGAGAGACTCTGTGAGGCAT | |
| | TCCGGCTTTACACCCCGTTTGACCCTGAGGCTGCTGAAAATCAGCG | |
| | CATGGTGAATACGGCATTTGTAGGGCAAGCCCAGGGTGACATCAG | |
| | GCGGAAGCTGCAGAAGCTAGAAGGTTTCGCAGGCATGAATGCCAC | |
| | CCAGCTTATAGAAGTGGCCACCAAGGTGTACGTTAACCGTGACCA | |
| | GGAGGCAAAAAGGGAAGCTGATCGGAGGCTTAGGAAAAAGGCCG | |
| | ATCTGCTGGCGGCAGCCCTCACGGAAAGGGAAGCTAGCATCGCGA | |
| | GGGGACGCGGACGCGGACGCGGACGTGGAAGGGGCCAAGCTGGG | |
| | CAGAGACCCGAGAGCCGGCCGAGGCTAGANAGGGATCAATGTGC | |
| | GCGGTGCAAAAAGAAGGGACACTGGAAGGATGAGTGTCCAGAGG | |
| | GCAATGAAGGAAATGGCCAAGGCCGTGAGACGAGAAGGCCGCCG | |
| | GCCAAGGGCTGCCGCACCCTGGGGGAACCAGACACCGACCTGATC | |
| | GGGCTGGCAGGGGCTGAAGGATATGAGGACTAGGACAGACCGGG | |
| | CTCCNTCTCCTTAGGCCCCCAGGAGCCCATGGTCACATTAGAAGTN | |
| | GGGGGCCAACTGATGGACTTTATGGTAGACACCGGGGCTGAACAC | |
| | TCGGTAGTGACCCGGCCCATAGGGCCACTATCCAAGAACTATACA | |
| | ACTATTGTNGGGGCTACNGGGGTCCCAGAGAAGAGGCCATTCTGC | |
| | CGGCCAAGGAGGTGTGTCATAGGAGGACGAGAAGTCCAACATGA | |
| | ATTCCTATACCTCCCAAATTGCCCAGTTCCCTTGCTGGGAAGAGAC | |
| | CTACTCCAAAAACTGCAAGCACAGATTGCTTTTGGGCCGCAAGGG | |
| | GATATGACTTTAAACCTGACTCACCCAAAGGCCATGGTGTTAACCC | |
| | TTACCGTCCCGCAGGCTGAGGAATGGAGACTATACGCAAAANAGN | |
| | CGCCAGAACCGGGANTAAATGAANTGTNTGGGCTACTTAGTAAAA | |
| | TTCCTGGAGTATGGGCTGAAGATAACCCACCCGGGCTGGCTGTAA | |
| | ATCAGGCACCGGTGGTAGTAGAGCTAAAACCGGGAGCAACTCCGG | |
| | TTCGGGTTCGCCAGTACCCGCTTCCCCGAGAGGCCGTACGGGGCAT | |
| | TCGCAAACACTTAGAGCGGCTCTNCAAACACGGAATCTTAGTCCG | |
| | ATGCCAGTCACCCTGGAACACTCCACTCTTGCCAGTACGGAAGCC | |
| | AGGGTCTGGTGAATATAGGCCGGTGCAGGACTTGCGTGCTGTAAA | |
| | CCAGGCCACGGTGACCATCCACCCGGTGGTACCAAACCCGTATAC | |
| | TTTAATGGGACTCATTCCGGCAAGTGCCGCCTGGTTTACNNGCCTA | |
| | GACTTAAAGGATGCNTTCTTCTGTCTCCGCCTGGCACCAATTAGTC | |
| | AGCCCATCTTTGCATTTCAATGGGACGATTNAGTCACAGGCACAG | |
| | NGGNGCAGCTCACCTGGACTAGACTCCCACAAGGGTTCAAAAACT | |
| | CTCCCACAATCTTTGGAGAAGCACTGGCCTCAGACCTCAAGGCCTA | |
| | CACCCCGCCAAATGACAACTGCGCCTTGCTNCAGTACGTAGACGA | |
| | CCTTCTTCTGGCAGCCCCAACCCGAGAGGACTGCTACCAAGGAAC | |
| | CCAAGACCTCCTCCACCTCCTGTGGAAGGCAGGNTATAAAGTGTC | |
| | CAGGAAGAAGGCTCAAATCTGCCNNGAAAGNGTCAAGTATTTAGG | |
| | CTTCATAGTAAGCCAAGGGGAACGNCGGCTTGGCAGTGAACGAAA | |
| | GCAGGCTGTTTGTGCACTTCCNACTCCAACCACCCGGCGTCAAAT | |
| | AAGAGAGTTCTTAGGGGCAGCAGGGTTCTGCCGCATCTGGATCCC | |
| | AAATTTCTCGCTNATGGCTAAGCCATTATATGAAGCCACAAAGAG | |
| | GGGGGAAAAGGAGCCCCTCCTCTGGGAGGCCGACCAGGAGAAGG | |
| | CNTTTAAACAAATCAAAGAAGCCTTAACTCAGGCCCCAGCCTTAG | |
| | GACTGCCAGATNTAACTAAGCCTTTCTTTCTGTATGTCCACGAGCG | |
| | AAAGGGAATGGCTATAGGGGTCCTGACTCAAGTCATAGGATCATG | |
| | GCATCGCCCGGTGGCGTACTTATCCAGGCAATTGGACTCCGTGGCG | |
| | CTNGGATGGCCTCCTTGCCTTAAGGCACTAGCTGCCACCGCCCT | |
| | ACTGGCACAAGAAGCTAACAAACTGACTCTAGGACAGCAACTGAC | |
| | CATCCGGGTACGCCGCACTCGGTTATAACTTTAATGGACCAGAGA | |
| | GGGCACCATTGGTTATCAAATCCGAGAATGACTCGGTACCAGGGG | |
| | CTCCTATGCGAAAATCCCCGCATAACTTTAGAAACNGTGAACACC | |
| | CTNAACCCGGCTACCTTGCTCCCGATCGAACCGGGAGNCCCCCTTC | |
| | ATNACTGTGTGGANACGGTAGATGAGGTGTTCTCAAGCCGGAGA | |
| | GATCTNACAGACCGGCCCCTCGGGGACCCGGACGTTGAATACTTC | |
| | ACAGATGGAAGCAGTTTCGTACTAGAAGGGGTCCGCCGAGCCGGG | |
| | TATGCAGTGGTGACNTTGGACTCAGTGGTAGAGGCNCAGCCTCTG | |
| | CCTACCGGAACNTCNGCCCAGAAGGCAGAGCTAATAGCCCTGACA | |
| | AGAGCTCTTTTGCTAGCAAAAGACAAAAAGGTCAATATTTACACN | |
| | GATTCCAAATATGCTTTTGCCACNTTGCATGTTCATGGGGCTATA | |
| | TACAAAGAAAGAGGACTCTTAACTGCGGAGGGAAAAAGAAATAA | |
| | AGTACAAGGAAGAAATTCTACAGCTCTTAGATGCTGTATGGGCCC | |
| | CAAAGAAGGTGGCTGTNATGCACTGCAGGGGGCACCAAAAGGCA | |
| | GGAACACTAGAGGCCAAAGGAAACAGAAAGGCAGACAGGGAGGC | |
| | AAAGCGGGCAGCAATGACTACTCCGCATTCTAAAGAGGAAGCCCT | |
| | AGCTATGCCTCTCCTCCCGGAGNCTCCCCTCCCGGAGATCCCAAGT | |
| | TACNCTCCAAATGAAAGGGCCTGGTTTGCCCAAGAANCTGGAAAN | |
| | TACATTGAAGGAGGATGGTGGAAATTCTCCGATGGGAGGCTAGCC | |
| | ATNCCTGAAATGGTGGCCCCCAAATTTGTAAAACAATTCCACCAA | |
| | GGAACTCACATGGGAAAAACGGCACTAGAAACGCTACTAGGACGC | |
| | CATTTCTATGTGCCACGGCTCACTGCCATCACTCGAGCCGTTTGCG | |
| | AACAATGTCTAACTTGTGCCCAGAACAACCCACGACAAGGGCCCA | |
| | CTCGGCCCCCCGGGAATTCAGGAAATAGGAGCCACGCCCTGTGAA | |
| | AACCTGCTTATGGACTTCACCGAACTGCCCCGAGCNGGGGGCTAT | |
| | CGGTACATGCTGGTGCTCGTTTGCACCTTTTCAGGATGGGTCGAGG | |
| | CTTTCCCCACCAGGACAGAGAAAGCACGAGAAGTGACTAAAGTAC | |
| | TGTTAAGAGACATTATCCCCAGATTTGGACTGCCTCTAACTCTAGG | |
| | GTCAGACAATGGACCGGCATTTGTAGCTGAAATAGTTCAGGANC | |
| | TAACACGGCTGTTAAAAATAAAATGGAAGTTACACACAGCCTACC | |
| | GGCCGCAGAGCTCAGGAAAAGTGGAGCGCATGAACCGGACACTC | |
| | AAGCAGCTACTGAAGAAATATTGCCAGGAAACTCATCTGAGATGG | |
| | GATCAGGTCTTGCCCATGGTCCTCCTCCGAGTCAGGTGCACCCCCA | |
| | CCAAACAAACTGGGTATTCGCCCTATGAGATTTTGTTCGGCCGGCC | |
| | ACCCCCAATCATAGGTCAAATTAAAGGTGATCTCCGNGAACTAG | |
| | GGGAATTAACCTTAAGAAGGCAAATGCAGGCTTTAGGGATAGCCA | |
| | TGCAAAANGTCCATGGCTGGGTACGGGAAAGAATGCCTATAAGCC | |
| | TGACAGACCCAGCACACCCCTTTAAACCTGGGGACTCTGTTTAGGT | |
| | TAAGAAATGGAATCCAACCACTCTAGGACCCATATGGGATGGGC | |
| | CCCATACTGTAATCTTGTCCACTCCCACTGCTGTTAAAGTTGCAGG | |
| | NATCGTGCCTTGGATCCACCACAGTCGGCTGAAACCGGCAGCCC | |
| | AGGACAAGTGGACCAGCCAGCAGGACCCAGACCATCCGACCCGG | |
| | CTGATCCTGCGACGAGACCGAGTTGCCGCTGAGAGACGACGACAG | |
| | CCCTGCTCTGGTCACTCCGGAAGCTGACCAGTCTACGCACGGCTGA | |
| | AGCTTGAGGAGACAACAGCCCTGCTCTAGTCACCCCGGAAGCTGA | |
| | CTAGTCTACGCACGGCCGAAGCTTGAGTANTCATCAAGNAAGTAA | |
| | ATGTGGTTAGAAATCTTAAGNCCAGTAGTTTTCCTTGTAATACTA | |
| | ATTGTTTTACTATTGTTCTGTCGCTTTGCTCAACCTCCTCCCCCGGG | |
| | TAAAGACCTCTTCTGTCCNTGCTGGGTATAAANATGCTACTCT | |
| | TTACTTNNTTGTTCCGGCGNNCGNNGCTACNNNTTTCCTCCTNATT | |
| | ATGNCACTCCCCTTANCCGTGTCAGAAGAAGAGCCCATAGAAGG | |
| | GTGCCCCCACTGCACNCACACTACNTGGTCAGGGAGCACNATAAC | |
| | TAGAACCCTGTTGTACCATACTTATTATGAGTGTACAGGGACCCG | |
| | CCTAGGAACTTGTACTCACAACCAGACGACCTACTCAGTCTGTGAC | |
| | CCAGGAAATGGCCAGCCTTATATATGTTATGACCCCAAGTCCTT | |
| | ACCTGGGACCTGGTTTGAAATTCATGTGGGTCAAAAGAAGGAAAC | |
| | CTTCTAAACCAAACCAAGGTCTCTCCCTCCCACGGGGGGGCTATN | |
| | TCCTTGTACTTTGATGTTTGCCAGNTAACATCCATGGGCTCAACCT | |
| | TTCCCGTAATCTCTAGTTCCGNAGAGTACTATAGNAGCTGCCAC | |
| | AAAAATATATGCGCACCCCCTGCTTGCTCCGCCAGGTCCCCAGAA | |
| | ACAGCTTGCTGGGACTGCACAATCGGTCCNCTGACCCGCAATCAC | |
| | CGGGGCCAGTCATGCTTACCAAAATGCCAGCAAAACCAGATTGTA | |
| | AGACAAGCACTTGCAATCCTGTAAATCTCACCATCTTAAAGCCGG | |
| | ATCTGCCCATATGGACTACAGGTTTANNNGCACCCCGGGANTACA | |
| | AGTCAGCGGTCAGAAAACNGACCCAGAAGCCTATTTATATATTAT | |
| | CAAGAAANCTCGGACCCGTCCGCCCGANAATTCCAGTCTTTAAGT | |
| | CATTCTNTGAGCATATNAACCAGAAGTTGCCNGAGCCCCCTCCTT | |
| | TGCCAGAAACCTATTCGNCGCGNNNCGTTTGCNTGCTCAGCTGGCT | |
| | GAAAACATNGCCGGCAGCCTAGGCGTCTCCTCATGTTATGTTTG | |
| | TGGAGGGACCAACATGGGAGACCAATGGCCNTGGGAAGCAAAAG | |
| | AGTTAATGCCCCAAGATAACTTCACTCTACTGNCTCTTCCCCCGAA | |
| | CCGACGCCCACAAGTTCGAGCGTCTGGCTCTTAAAAACCTCTATTA | |
| | TCGGGAGATNCTGCGTTGCTCGCTGGGGAAAAGCCTTTACAGAC | |
| | CCAGTAGGAGAATTAACCTGCCTAGGACAGCAATATTACAATGAA | |
| | ACACTAGGAAAAACTTTATGGCGGGGCAAAAATAATTCCAAATCG | |
| | CCCCANCCAAGCCCATTCTCCCGTTTCCCTTCTTTAAACCACTCTTG | |
| | GTACCAACTTGAAGCTCCAAATACCTGGCAGGCGCCCTCTGGC | |
| | CTCTACTGGATCTGTGGGCCACGGGCATATCGGCAGTTGCCGGCTA | |
| | AATGGACAGGGGCCTGTGTACTNGGAACAATTAGGCCGTCTTTC | |
| | TTCCTAATCCCNCTGNAACAGGGAGAAGCTTTAGGGTACCCCGTCT | |
| | ATGATGAAACTAAAAGGAGAAACAAAAAGAGACATAACCATAGG | |
| | AAATTGGAAAGACAATNAATGGCCCCCTGAAAGAATAATCCAATA | |
| | CTATGGGCCAGCCACCTGGGCAGAAGATGGGNCGTGGGGATACCG | |
| | CACCCCTATTTACATGCTCAACCGCATCATAAGGTTGCAGGCAGTG | |
| | CTTGAAATCATCACTAATGANACNGCAAANGCCTTAGATCTGCT | |
| | GGCCCAGCAAGCCACAAAAATGAGGAACGCTATCTATCAAAATAG | |
| | ACTGGCTTTAGACTACCTCCTAGCCCAGGAAGGAGGAGTATGTGG | |
| | AAAGTTCAATCTAACTAATTGCTGCCTAGAAATCGATGACAACGG | |
| | AAAGGCCATTANGGANATAACTGCAAGAATNCGAAAATTAGCCCA | |
| | TGTTCCAGTCCAGACTTGGAAAGGGTGGTCTCCAGATTCCCTCTTC | |
| | GGAGGCTGGTTTTCATCCTTCGGAGGATTCAAGACCTTAATAGG | |
| | AGTAGTTCTGGCCATACTAGGAGNCTGCCTAATACTCCCTTGTCTC | |
| | TTACCCCTCCTTGTTAGAAGCATTCAATCAGCCATAGAGGCNNT | |
| | TGTAGCCAGGCAAACTACCACTCAGCTAATGGCTCTAACTAAATAT | |
| | CAACCTTTGCCAAANGAAGAAAACTGCCTCTTCATGAAGAATTA | |
| | AGTAATAGTGATGCTTTCTATTAAACTTCATTTATAAAAAGCATCA | |
| | AAGGGGGGAA | |
| 20 | AGTGGCGTCCGAACACAGGGACTTCGAGGACGTGAACGAAGAAG | Consensus sequence |
| | GTCTGCTGGAGCAGAGGAACTGAAATTGACAAGGCGAACGGGGA | for transposable |
| | CCCCGGGACGAGTCTGCCGGCAGCGGATATAAGGTCAGTGCCCTA | elements that belong |
| | AAGAGGTACTGGGAGCAATATAAGGTCAGTGCCCTAAAGAGGTAC | to HERVK3 |
| | TGGGAACGGGAAGTTTTCTGAATCAGNGGTAACATGGGGCAGAAT | subfamily |
| | TTGTCTATTGAAGAAAAACATTATCGTGCAGTTGCTTAAAGTTCTG | |
| | TTGAGACAGTCTGGNGCTCAGGTTAGTTCNCAGACACTAACTAAG | |
| | ATGCCGCAGGAGGTTATTACGCATAACCCATGGTTTCCACAGGCA | |
| | GGCACTCTTGATGTGGAAAATTGGGACAGAGCAGGAGAAGGATTA | |
| | AAACGGGCTCATCAAAAAGGTCTTAAAGTTGATTCTTCTGTTTTCT | |
| | CCACTTGGAGTTTAGTTCGTACTGTNCTTCTGCCATTATCTCCTTNT | |
| | TATTCTGCNGGACAGCAGGAGTCATGTTCTGAGTCTAAAAATC | |
| | TGAAAGAATCTGTTGTCCCACCCACAGCTCCAATTGAAAATAAAA | |
| | AACAGGAGAGGGAGGATAAAAATTGGCCTATACCGCCTCCTCCAG | |
| | TTGCAGAAACATCTGTACCGCCTCCTTCGGTAGCAGAAATAGAGA | |
| | CCCCAATACAAAGAATTTTACGCTCTGCTGCCATAGCTGGAGAGC | |
| | CCTTAGGACCTNTGCGCTTTTCCTATTTCCGTAAGGCCTGATCCAA | |
| | ATAATCCACAGCAGNTTATTCATGAACACACCCCACTAGAGTTT | |
| | AAGTTGTTGAAGGAATTAAAAGCTAAGTGTGGTNAATAATGGCGT | |
| | ACAGAGCCCATTCACTTTAGGATTGCTAGAATCTGTGTTTGGTGC | |
| | TATGCGTCTTCTACCCTTTGATGTAAAACACTTGGCNCGAACTTGC | |
| | TTGTCTGCTAGTGCATATCTGACATGGAATTTAAATTGGCAAGA | |
| | AATGTGTGCAGACCAGGCTAGACAGAACCGTGCTGCTGGACACGG | |
| | AGACATTACAGAGGATATGCTGTTAGGTAATGGCCCTTNATTCAG | |
| | ACCTGGAACGTCAAATGGCACTCCCAGACGCTGCTTATCAGCAGT | |
| | GTGCACAGGCCGCTAAACGCGCCTGGGCCACAATTCCTGAAGAGG | |
| | GAGTCCCAGTACAATCCTTTTTACATATCATGCAAGGGTCGCAGGA | |
| | ACCCTATGCGCAATTTCTTGCAAGATTACAAGAGGCAGTGAAGC | |
| | GTCAGATTCCTCATACCGCTGCCGCAGAAATGCTAACCTTAACTCT | |
| | AGCTTTTGAGAATGCAAACGCGGATTGTAAACGTGCACTGGCAC | |
| | CTGTGAGGTGTAAAAAAACTTGGGAAATTTTCTCAGAGCTTGTCAG | |
| | GATGTAGGAACTGAGCTTCATCGCTCTGCAATGTTAGCNCAAGC | |
| | AATGGCTAATTTAGCAGTTGACAAATCTAAAAGGAGCCAAGGGTC | |
| | AAACCCTAAAATGGGAAAATGTTATAATTGTGGAAAAACTGGACA | |
| | TTTTAAAAAGGAATGCCGCCAGATCTCAGGACAGAAAGGACCTTA | |
| | CAATGCAGTNCCCCCCACCCCCGCGNNCCAGCGGAAAAAAACGCC | |
| | AGGACTTTGTCCTCGCTGTAACAAAGGAAATCACTGGGCTAATCA | |
| | GTGCCGCTCAAAATTTCATCAGAATGGCACCCCCCTGTCGGGAAA | |
| | CGAGANGGGGGCCTGGACCCGGGCCCCTCAAACAATGAGGGCATT | |
| | CCCAGTCCAGACCACAACCCCGTTTCAGGGATGGGTTCCCGGAGG | |
| | NACATTGATTCCCTCACCCCAGGAACACCAGGAAGTGCAGGATTA | |
| | GATCTCCCCGCCAGAGAAAGAATTACGTTAGTTGGNGGAGACAAA | |
| | CCCACCAAAGTTCCCACTGGCATTTGGGGACCTTTACCAGCAGGAT | |
| | ACATGGGACTAATTTTAGGCAAAAGCCGCCTTAACTTGCAAGGC | |
| | ATTACTGTAGTCCCAGGAGTNGTTGACTCCGATTATGAAGGAGAA | |
| | ATTCAAGTAGTTTTAATGTCACAAGATCTTTGGGTTTTTGAACCG | |
| | GGAGAATATATTGCTCAATTATTGCTTATTCCCTGCAAATTACACC | |
| | CTTCTCCACGAAAGGAGAAACGAGGAAATAAAGGGTTTGGGAGC | |
| | ACAACTACATGGGAAATCTATCTATCCNCAACCCATAGCCTCTAAT | |
| | AGACCCACCTGTGTAGTACAAATTAAAGGAAAGAAATTTTATGG | |
| | GCTTATGGATACGGGAGCTGATGTGTCAGTAATATCTAGNAACGA | |
| | CTGGCCCCCATCCTGGCCCCTGCGATTAACTTCTACATCCCTAGT | |
| | GGGAGTAGGAACAGCTCAAAGTGTTCAACAGAGTGCTGAGATTTT | |
| | ACCTTGTCTTGGTCCGGATGGACAGTCATGTACTTTTCAGCCTTA | |
| | TGTTGCAAATATAGCTATCAATTTATGGGGTCGAGACTTACTTACA | |
| | GCATGGGATATGAGACTTACAAATGAAAACTTTGATAACCCAGG | |
| | ATTTAAAATGTTGAAGGACATGGGATATCAGAGTGGAAAAGGTTT | |
| | AGGGAAATTCCTACAAGGAAACCCTAACCCGATATCAGTAACTGG | |
| | AAAAACAGATAGAAAAGGGCTAGGACGTCAGGATTTCTGACGGG | |
| | GGTCATTGATATTTCTCCTCCGCCCACTGCCTTACCATTAGAATGG | |
| | CTNAGTGACAAACCTGTGTGGGTGGATCAATGGCCCCTANCACAG | |
| | GAGAAGCTAGNTCAACTTCATCNGCTAGTAAAAGAGCAATTGGAT | |
| | GCAGGACATATAGAGAAGAGTTNCAGCCCCTGGAATTCACCGGTG | |
| | TTTGTTATTCCAAAAAAGTCCGGAAGATGGTGACTGCTGCATGAT | |
| | TTGAGAGCTATTAATGCACAAATTAAACCGATGGGTGCATTACAG | |
| | CAAGGTCTGCCATCCCCAGCGGCCATTCCAAGAGACTGGCCTCTC | |
| | GTAGTAATAGATCTTAAGGATTGTTTCTTTACTATACCNTTACACG | |
| | AGAAGGATAAGCCTCGATTTGCCTTCTCTGTGCCTTCTATTAAT | |
| | CAAAGAGAACCTGTTTCTCGTTATCAATGGAAAGTTTTACCCCAAG | |
| | GCATGCTTAACAGTCCTACGCTATGTCAGCATTTTGTAGGACGG | |
| | GCATTAAAGGAGCCTCGGAATATGTTTCCCACTGCTTACATCATTC | |
| | ATTNTATGGATGATATTCTTTTGGCCGCTCCTACAGATCAAATC | |
| | TTACATCAGTTATTCAGAGAAACAAAGCGGGCTTTGACTAAATGG | |
| | AATCTCAAAATNGCTCCAGAGAAGGTGCAAACAACTTCCCCATAC | |
| | CANTACTTAGGAACTATTGTTACGGAGAGAAGTGTACGGCCTCAG | |
| | AAAGTAGTTCTCCGTAAAGACAGGTTACAGACTTTAAATGATTTT | |
| | CAACAATTATTAGGGGATATTAATTGGCTGCGCCCGATGCTAGGTA | |
| | TTGCTACCTATCAACTTACACATCTTTACCAAACCCTGCAAGGA | |
| | GATTCTTCNTTAAATTCCCCGCGGCAACTNACTAAAGAGGCAGAA | |
| | GCCGAGTTACGGCTTGTAGAGCAGATGCTTCAGCAGAGACATGCC | |
| | TCNCGGCTACAGCCGCAAAAACCTTTGCTTTTGTTTATTCTTCCTAC | |
| | CCCCCACTCTCCAACAGGACTTTTGGGCCAGTTCATAGACAAG | |
| | TCTGTAACAGTAATAGAATGGCTCTTTCTACCTAATCAGTCAAAAC | |
| | CTTGCAAGTTTATCTTTCTTTAATTACACAAATTGTGACTATGG | |
| | GCAGGCATAGGTCAAAAATGCTTACGGGATATGATCCNGACAAAA | |
| | TTATTGTTCCCTTAGACTCCCAGCAACAGGCCGCAGCNTGGGAAA | |
| | TGTCGACTGCNTGGCAAATCGCTTTCGCAGACTTCGTGGGAATAAT | |
| | AGATAACCACTATCCCTCAGACAAAATTTTGCAGTTTTATAAAG | |
| | TCCATTCTTTCATTCTTCCTGTGATTACTCATCACAAGCCTATTCCA | |
| | GGTGGACAGACTTATTTTACTGATGGCTCTTCCAAAGGTCGTG | |
| | CAGCTATCTATGGACCTAAACATACTCAAACAATAATGACCTCTGG | |
| | GGTTTCAGCTCAACGCTCAGAGCTAATTGCAGTCATTCAGGTTT | |
| | TACAGCTCACAGCTTCAGATCCTATCAACATTGTCTGTGATTCAGC | |
| | TTATGTTGTAAATGTAGCCAGTCGCATAGAAACTGCTACAATTA | |
| | AAAGTACACTAGACCCAGAACTGCTTAATTTGTTTCTAAGACTTCA | |
| | NACAGCTATTCGCTCTCGTGCAGCTCCTTTTCATATTTCTCATA | |
| | TTCGCTCTCACACACAACTTCCTGGACCACTATCTCTAGGTAATGA | |
| | TAGAGCAGATAAACTGATTGGTTCTGTGTTTCAGCAAGCTCAAG | |
| | CNTCTCNATGCGCTACTGCACCAAAACACCTCCGCCCTTACTCGCA | |
| | TGTTTCATCTGCCTCGCAGCCAAGCTAGGGCTATNGTACAAGCC | |
| | TGTCCTACTTGCCAGCATGTCCCTGGNGCCGCACCTGTAGAAGGNT | |
| | GTAACCCACGAGGTTTGGCTCCAAATGAAATCTGGCAAATGGAT | |
| | GTTACACACATAGCAGCCTTTGGCAAGCTTAGCTATGTTCTGTGAN | |
| | CTATAGACACTTATTCTCATATGCTGCATGCTACATGCCAAACA | |
| | GGTGAGACAGCTGGTCATGTACGGCGACATTGTCTGTCATCATTTG | |
| | CTCATATGGGGATACCTAAACAATTAAAAACTGACAATGGACCC | |
| | GCTTATACTAGTCATGCTTTTCAAAATTTCTTACAGCTTTGGGCTAT | |
| | AACCCATAAAACAGGAATTCCTTATAATCCTAGAGGACAAGGC | |
| | ATTATAGAGCGGGCACATCAAACATTACAACGCATGTTGAAAAAA | |
| | CAAAAAGGGNGGTATAGGAGGCCAACTACCACCTCAATCAAAACT | |
| | ACATTTAGCCTTATTTACTTTAAATTTTTTNGACTCCTGGTACGGAT | |
| | GGTAAGACTCCAGCAGAAAGACATTGGCAAGTGTTAGAGGAAA | |
| | AGAGGAAAGTTTATCCGAAAGTGTTATGGAAATCCCCGGAAGAAG | |
| | NGACAATGGAAAGGTCCGGTGGATTTACTGACGTGGGGANGAGGG | |
| | TATGCTTGTGTTTTTACAGGAGATGGACAAACCGTGTGGGTGCCCT | |
| | CAAGGTGCGTGCGACCATGGAACGGGAGACTGGAGGAACCCAGG | |
| | GTGGCCAACCATGGGCCCGGTCCCTCCGGTACGAGCCATGAGCCA | |
| | GCTGAGCCTGAGTGCAAAGACGGAGAGAAGGCCGACCGGAGTCA | |
| | CGACGACATCAACCCCCATAACCTGGGGACAACTCAAGAAAACCA | |
| | CGCAGGAAGCTGAGAAACTACTGGAGCGTCAGGGNCAGGCAAAA | |
| | ACCCCTGATTCCATGTTCTTGGCCATGTTAGCCATAATGTCCTGTG | |
| | CGGTATGTTTTCCCTGTGCAGAGGCAAAAACATATTGGGCATATGT | |
| | TCCCAATCCCCCAGCAGTACGACCTGTACTTTGGAGTGACACTCCT | |
| | CCTGAGATTTATCATGATCAGGGAGCGTGGGCTCCAGGACCCCT | |
| | AACTCCCCTGACANTAGAACAGTTAGACTCTCAGAACAATGTCAT | |
| | CAATTATACCGCTCCACTGGAAGGACTCCCTTTGTGTATCACCAC | |
| | AAAGACGTCGCTCAGCCGTAGCTGTCTTACAATTCAAGCTCAAGC | |
| | ATGGTTGAGTCACTATGGAAAAGTCATGTACTTATTAGGTCTTGG | |
| | TTCTATTAATGTAACTGGTGTGCTAACCAACCATTCCCGGCCCAAT | |
| | CGCCCTAATTGTGCTGACTATACGGAATGGATTCCCTTCAATAG | |
| | TTCCTACCCCCCCTCNCGTGGACCCAGTGTCTTGGCCCACTGGCTA | |
| | GAAAACAATCTATGTTAACTGGAGACATTGTGGATTGGGGACCT | |
| | AAAGGTCAATTAGATGGAAAAGATGAAAATCAGAAATCATGGCAC | |
| | AAACTTCGCTGGCATTGGTGGCAAGCTTTTAATGCTTCTTCTTTA | |
| | TACNACACCGGGATCCAATCCCAGTCTGCCGCCCAGATTGCTTGGC | |
| | ATGGAGCAGGCTTTAGCCCGCCTCTTCCTCAGTGGCATTATCTA | |
| | GGGAGGAAAGGACCAATTCAAGAGACGATATGGAAGGCAGCACT | |
| | CCCATTTACGAATGGAGCATCTGGGTTNGGGATACTATCCAATAAT | |
| | AGCAATAGTAAGCGACACAGTCTTAATGTTACATTTGTAAAGAAT | |
| | ATCACCACTCAATTTACGGTTTGTGTTTTTAATCCTTATGTCTTT | |
| | TTGGCAGCTAAGAAGGACCAGCTCCAGGTAAACAATACCCAATTG | |
| | ACCTGTAAATCTTGCCAGTTATATCACTGCATTAATCATAGCACA | |
| | TTGCAAACACATAATATCTCTACTTTGATGATTTTGGGTCGCATCC | |
| | CTGGGCTATGGATTCCTGTTAATCTGTCCGAGCCTTGGGCTGCC | |
| | ACACCTGCTTTGCATTTTGTGAAACTTCTTCTAACTCAGCTTACTCA | |
| | TCGTGTCCGTAGAGCCTTAGGCATGATAATTTTTGCTATTGTT | |
| | TCCTTGGTCACACTAATAACTTCTGTTGTGATGTCCTCTGTAGCTTT | |
| | GCATAGTTCTGTTCAAACAGCTCAGTACGTGGAGAACTGGACG | |
| | CGCACAGCCGACCAAGCGTGGCTACTTCAGAATAAAATTAACACT | |
| | GAGTTACAAACTGAAGTGGCAATGTTGAAATCCACGGTTCTATGG | |
| | TTAGGGGAACAAGTACAAAGCTTGCAGTTGCAGCAGCAATTGCGT | |
| | TGTCATTTTAATCACACTCATATTTGTGTAACCAACTTAGAATAT | |
| | AACCAAAGTGAGTATCCGTGGGACCTTGTGAAAGCCCATTTGCAG | |
| | GGAGCTTTCACATCCAACATCACCTTTGATATTGGTGAATTACAA | |
| | AACAAAATTCTTGATTTAAATAGGCAAACTCAAGAGTTTCAGCCTT | |
| | CTTTAGAAGACTGGACCGAATTCCAGCAAGGCCTGGAGAGCCTC | |
| | AACCCTTGGACCTATCTAAGGCACCACATTAACATCTTATATGTAG | |
| | TTCTTGGAATAATGTTGTTTTGTCTCTGTCTTCTGTTCATAGTC | |
| | TGTAAAATCGGATGGACCGCCAATCGGAGAATGAGAGCTGCCCAG | |
| | CCTGGCCTTACATTCTTTCAATTAATNCATAAACAGAAAGGGGGA | |
| | TA | |
| 21 | GATTTTGGTACCAGGAGTGGTTCTAGAGGAACAGAATATTAAGGA | Consensus sequence |
| | TGGAGTTCTTTCGTTGGTTTTGGGGTTTCTGGAGTTGGCTGCTTA | for transposable |
| | ATATGATTAGACCCAAAAATGCTAAGGACTCTACTTCTAATAGTAT | elements that belong |
| | GGAGAACACTGATAGTCCTTGGCGTGAACTGTTTAGAGAGTTAT | to HERVL |
| | GCAAAATAAATGCATTTGACACTCCTGATTCACCGCTCGTGAGAG | subfamily |
| | GCAAGGAGTTTAGTGACTCTATACATAATACCTTTGACCATATGT | |
| | GGAGAACCAAGGAACATAATGAAGCTGGTTGGTTGCTCCTAAGTT | |
| | CAGTGGACAAAGTGATGAAAGAAAATGATGAACTCAGGGATTCTA | |
| | TCTCCCGGCTTCAGAAGCAGATACTGAGCCTCAAATCTGCTAAGAT | |
| | TGCCCTGAGTGAGAGTCTTATCTCCTGTAGAGAAAGAGCTGAAA | |
| | TTGTGGAAAAACAGACACAAGCTCTTATCATGCGAGTGGCTGACC | |
| | TGCAACGAAAGGTGCATGCACAGCCTCGCCAGGTGTCTACTGTTA | |
| | AAGTGAGGGCATTGATTGGAAAAGAATGGGACCCTGCAACTTGGA | |
| | ATGGGGACGTGTGGGAGGACCCTGATGAAGCTGGGGACACTGAGT | |
| | TTGTAAACTCTGATGAACCTTTTTTGCCAGAAGAAACAGCTTCCCC | |
| | ATCCCCAGTAGTGGCAACATCCCCTCCCCGACCCATGCTGCCAT | |
| | CAGCCTTTCCACCTTTGTCTGAGGAGATAAACCCTGCGCTGCCTGA | |
| | GGCAACAGTGATGGCCTCCCCTGAGGCAGTTGCCAGGCAAGATA | |
| | ATGTTGATTCTCCTCAGGAGCCACCCCCAACACCCCTGTTTGCTTC | |
| | TAGACCTATAACTAGACTAAAGTCCCGGCGGGCCCCTAGAGGTG | |
| | AGGTTGAGAGTGTGACCCATGAGGAGGTGCGCTACACTCGAAAAG | |
| | AACTGCTTGAGTTTTCTAATTTATATAAACAGAAATCTGGAGAAC | |
| | AGGCATGGGAATGGATATTAAGGGTGTGGGATAATGGTGGAAGGA | |
| | ACATAGAGTTGGATCAGGCTGAATTTATTGATTTGGGCCCACTAA | |
| | GTAGGGACTCTGCATTTAATGTTGCAGCTCGGGGAGTTAAAAAAG | |
| | GTTCTAATAGTTTATTTGCTTGGTTAGCTGAAATATGGATTAAAA | |
| | GATGGCCCACTGTGAGCGAGCTGGAAATGCCTGATCTCCCTTGGTT | |
| | TAATGTAGAGGAAGGGATCCAAAGGCTTAGGGAGATTGGGATGG | |
| | TGGAGTGGATTAGTCACTTTAGACCTACTCATCCCAGCTGGGAGGG | |
| | TCCAGAAGATATACCCTTGACCAATGCCTTGCGAAATAGATTTG | |
| | TGAGGGCAGCACCTGCATCTTTGAAGAGCCCTGTAATTGCTCTTCT | |
| | CTGTATGTCAGATCTAACAGTGGGAACCGCAGTCACTCAACTAC | |
| | AAAATTTAAATACAATGGGAATAATTGGATCCCGAGGTGGCAGGG | |
| | GCCAAGTGGCGGCACTCAACCGTCAAAGGCAAGGTGGGCGTAGCT | |
| | ACCGTAATGGACAGCAGAGGCAAAGCGGCAATCAGAATAGTCTGA | |
| | CTCGTGTAGAGCTCTGGCATTGGCTAATTAATCACGGTGTTCCTA | |
| | GAAGTGAAATTGATAGGAAGCCTACTGCATTCCTACTTAATTTATA | |
| | TAAGCAGAAAACTTCTAGGTCGAATGGACAAAAGACTAATTTGA | |
| | ATTATAAAAACAGAGAATCACGGCCCCTCAATCAATTTCCAGACTT | |
| | GAGCCAGTTTACAGACCCAGAACCCCTTGAATGAAGGGGAGGCC | |
| | GGGTCCCCTTGAGGAAGGACCCCACTACACTACCGACAATTTATG | |
| | CNGTGAATCTTTCTCCCATCCTTCCCCAAGGAGACCTCCGGCCTT | |
| | TTACCAGGGTAACTGTGCACTGGGGAAAGGGAAATGATCAGACAT | |
| | TTCGGGGACTACTGGACACTGGCTCTGAGCTGACGTTGATTCCAG | |
| | GGGACCCAAAACGTCATTGTGGTCCTCCAGTTAAAGTAGGGGCTT | |
| | ATGGAGGTCAGGTAATTAATGGAGTTTTAGCTCAGGTCCGACTTA | |
| | CAGTGGGTCCAGTGGGTCCCCGGACTCATCCTGTGGTCATTTCCCC | |
| | AGTGCCAGAATGCATAATTGGCATAGACATACTTAGCAGCTGGC | |
| | AGAACCCCCACATTGGCTCCCTGACTGGTAGGGTGAGGGCTATTAT | |
| | GGTGGGAAAGGCCAAATGGAAGCCATTAGAGCTGCCTCTACCTA | |
| | GAAAAATAGTAAATCAAAAACAATATCGCATCCCTGGAGGGATTG | |
| | CGGAGATTAGTGCCACCATCAAGGACTTGAAAGACGCAGGGGTGG | |
| | TGATTCCCACCACATCCCCGTTCAACTCTCCCATTTGGCCTGTGCA | |
| | GAAGACAGATGGATCTTGGAGAATGACAGTGGATTATCGTAAGC | |
| | TTAACCAAGTGGTGACTCCAATTGCAGCTGCTGTACCAGATGTGGT | |
| | TTCATTGCTTGAGCAAATTAACACATCTCCTGGTACCTGGTATG | |
| | CAGCCATTGACTTGGCAAATGCCTTTTTCTCCATTCCTGTCCATAA | |
| | GGCCCACCAGAAGCAATTTGCCTTCAGCTGGCAAGGCCAGCAAT | |
| | ATACCTTTACTGTCCTACCTCAGGGGTATATCAACTCTCCGGCTTT | |
| | GTGTCATAATCTTATTCGGAGAGACCTTGATCGCTTTTCGCTTC | |
| | CGCAAGATATCACACTGGTCCATTACATTGATGACATTATGCTGAT | |
| | TGGATCCAGTGAGCAAGAAGTAGCAAACACACTGGACTTATTGG | |
| | TGAGACATTTGCGTGCCAGAGGATGGGAAATAAATCCGACTAAAA | |
| | TTCAGGGACCTTCTACCTCAGTAAAATTTCTAGGGGTCCAGTGGT | |
| | GTGGGGCCTGTCGAGATATTCCTTCTAAGGTGAAGGATAAGTTGCT | |
| | GCATTTGGCCCCTCCTACAACCAAGAAAGAGGCACAACGCCTAG | |
| | TGGGCCTATTTGGATTTTGGAGGCAACACATTCCTCATTTGGGTGT | |
| | GTTACTCCGGCCCATTTATCGAGTGACCCGAAAGGCTGCCAGTT | |
| | TTGAGTGGGGTCCAGAACAGGAGAAGGCTCTGCAACAGGTCCAGG | |
| | CTGCTGTGCAAGCTGCTCTGCCACTTGGGCCATATGACCCAGCAG | |
| | ATCCAATGGTGCTTGAGGTGTCAGTGGCAGATAGGGATGCTGTTTG | |
| | GAGCCTTTGGCAGGCCCCCATAGGTGAATCACAGCGGAGGCCTC | |
| | TAGGATTTTGGAGCAAGGCCCTGCCATCTTCTGCAGATAACTACTC | |
| | TCCTTTTGAGAGACAGCTCTTGGCCTGTTACTGGGCTTTGGTGG | |
| | AAACTGAACGTTTGACTATGGGTCATCAAGTCACCATGCGACCTG | |
| | AACTGCCTATCATGAACTGGGTGCTTTCTGACCCATCTAGCCATA | |
| | AAGTGGGTCGTGCACAGCAGCATTCCATCATCAAATGGAAGTGGT | |
| | ATATACGTGATCGGGCTCGAGCAGGTCCTGAAGGCACAAGTAAGT | |
| | TACATGAGGAAGTGGCTCAAATGCCCATGGTCTCCACTCCTGCCAC | |
| | CCTGCCTTCTCTCCCCCAGCCTGCACCGATGGCCTCATGGGGAG | |
| | TTCCCTATGATCAGTTGACAGAGGAAGAGAAGACTAGGGCCTGGT | |
| | TCACAGATGGTTCTGCACGATATGCAGGCACCACCCGAAAGTGGA | |
| | CAGCTGCAGCACTACAGCCCCTTTCTAGGACATCCCTGAAGGACA | |
| | GCGGTGAAGGGAAATCTTCCCAGTGGGCAGAACTTCGAGCAGTGC | |
| | ACCTGGTTGTGCACTTTGCATGGAAGGAGAAATGGCCAGATGTGC | |
| | GATTATATACTGATTCATGGGCTGTAGCCAATGGTTTGGCTGGAT | |
| | GGTCAGGGACTTGGAAGAAGCATGATTGGAAAATTGGTGACAAAG | |
| | AAATTTGGGGAAGAGGTATGTGGATGGACCTCTCTGAGTGGTCAA | |
| | AAACTGTGAAGATATTTGTATCCCATGTGAGTGCTCACCAACGGGT | |
| | GACCTCAGCAGAGGAGGATTTTAATAATCAAGTGGATAGGATGA | |
| | CCCGTTCTGTGGACACCACTCAGCCTCTTTCCCCAGCCACCCCTGT | |
| | CATCGCCCAATGGGCCCATGAACAAAGTGGCCATGGTGGCAGGG | |
| | ATGGAGGTTACGCATGGGCTCAGCAACATGGACTTCCACTCACCA | |
| | AGGCTGACCTGGCTACGGCCACTGCTGAGTGCCCAATTTGCCAGC | |
| | AGCAGAGACCAACACTGAGCCCTCGATATGGCACCATTCCTCGGG | |
| | GTGATCAGCCAGCTACCTGGTGGCAGGTTGATTATATTGGACCTC | |
| | TTCCATCATGGAAAGGGCAGAGGTTTGTCCTCACTGGAATAGACA | |
| | CTTACTCCGGATATGGGTTTGCCTATCCTGCACGCAATGCTTCTG | |
| | CCAAGACTACCATCCGTGGACTCACGGAATGCCTTATCCACCGTCA | |
| | TGGTATTCCACACAGCATTGCCTCTGACCAAGGCACTCACTTTA | |
| | CGGCTAAAGAAGTGCGGCAGTGGGCTCATGCTCATGGAATTCACT | |
| | GGTCTTACCATGTTCCCCATCATCCTGAAGCAGCTGGATTGATAG | |
| | AACGGTGGAATGGCCTTTTGAAGTCACAATTACAACGCCAACTAG | |
| | GTGACAATACTTTGCAGGGCTGGGGCAAAGTTCTCCAGAAGGCCG | |
| | TGTATGCTCTGAATCAGCGTCCAATATATGGTACTGTTTCTCCCAT | |
| | AGCCAGGATTCACGGGTCCAGGAATCAAGGGGTGGAAGTGGAAG | |
| | TGGCACCACTCACCATCACCCCTAGTGATCCACTAGCAAAATTTTT | |
| | GCTTCCTGTTCCCGCGACATTACGTTCTGCTGGCCTAGAGGTCT | |
| | TAGTTCCAGAGGGAGGAACGCTGCCACCAGGAGACACAACAACG | |
| | ATTCCATTAAACTGGAAGTTAAGATTGCCACCTGGACACTTTGGGC | |
| | TCCTCCTACCTTTAAGTCAACAGGCTAAGAAGGGAGTTACAGTGTT | |
| | GGCTGGGGTGATTGACCCGGACTATCAAGATGAAATCAGTCTAC | |
| | TACTCCACAACGGAGGTAAGGAAGAGTATGCATGGAATACAGGAG | |
| | ATCCATTAGGGCGTCTCTTAGTATTACCATGCCCTGTGATTAAGG | |
| | TCAATGGGAAACTACAACAGCCCAATCCAGGCAGGACTACAAATG | |
| | GCCCAGACCCTTCAGGAATGAAGGTTTGGGTCACTCCACCAGGAA | |
| | AAAAACCACGACCTGCTGAGGTGCTTGCTGAAGGCAAAGGGAATA | |
| | CAGAATGGGTAGTAGAAGAAGGTAGTCATCAATACCAGCTACGAC | |
| | CACGTGACCAGCTGCAGAAACGAGGACTGTAATTGTCATGAGTAT | |
| | TTCCTCCTTCTTTTGTTAAAAACATGTTTGTGCATGTATACACTT | |
| | GTACTAAGAAAATATCTTCATTTTATTTCCTTTCTCCTTTATCATGT | |
| | GACATAAGATTTATTGACTTCATATCAGCATTTAAGTATTGTT | |
| | AACTTTATGTAATAGTATTTGGGTTGGGGATTGGTGCGTTTCCGGT | |
| | TGTACGAAGGATAGTTGTATTATGTTAGGCGTAATTATGACCTT | |
| | ATTATTGTCTTTATTTGAAGATTATGTATGATCTCAGGAGATGTGT | |
| | ATGGGTTCAAGTTGACAAGGGGTGGACT | |
| 22 | ATAGTCGGCAGGATCCCGAGGTGAGTGAGCCTTCGGCCCCCGATG | Consensus sequence |
| | ATCCCGGGTCGGCCATGTGGCCGCAGCATGGGCTGTGGTACCCGG | for transposable |
| | TGGCAGCCGTGCTGCTCGGATGGGCCCCGGTGGAAACNTGGGCGG | elements that belong |
| | CGGTGGACGGGTCCCCCGCGAGCGTGGAGAAGGCGCTGAAGCAGC | to HERVL18 |
| | TGGAAGCGCAGAGCACCGAGAAGGAGCGCGCCTTTGCCGGCAGA | subfamily |
| | GTCGGATGGGCGTTTTTGACTGCGCTACGGGAAGTACACGCCCAG | |
| | TCCCTGCGGGATGCAGCGCAGGTAAGGGACCTCCAGGCGCAAGCG | |
| | GGGCGCCTGGAGGCCCGGNTACACAGCTCGGAANAAGANTTGGG | |
| | GGTAAGGGACCTCCAGGCGCAGGCGGGGCGCCTGGAGGCCCGGNT | |
| | ACACAGCTTGGAANAGGAATTAGAGGCTGCCGTNAGTGCGGGCCT | |
| | GGGCCCGTCGTCCCGGCCGGAGACCCCCGCTCGGTCTGATACCGA | |
| | GGAGGAAGAACCCCCCGTTGCGGGCTCGCCCAGTGGTCCGTCAGA | |
| | AGGTAGANCATGAACAGCCGNTGGGGCCCCAAGGGCGGGCTCAG | |
| | GGACCCCCCACCGTGACGGAACACACTTCATATAGTGCCTATACCC | |
| | CAACTGAGTTGCGGGAATTAGGCAAGCAGTGCCGGCAGCGTCCGG | |
| | GGGAACCCCTGCCCGCCTGGATGCTTCGTCTNTGGGACGAGGGAG | |
| | CNGACAGTATTTCCTGCTCCGCCTCTGAGATGGAAAAGCTGGCCTC | |
| | TATCACGACTCACCCCTCCCTCCGTCAGCGGTTGCAGGTGAGCAGG | |
| | CGGTTGGCACAAGGGCAAGGTGACCACACCCTGATTGAGTGGCTG | |
| | ATGGCAGCCATACGGACTGTGTGGAACGACGCCGGNGAAATACCA | |
| | GAAACTGTGAGTAAATGGCAGTCGTATACCGATTTGGTGCAAGTN | |
| | ATCCGGGAGATGGGTATGCGGCAGGCTATGTTTGATCTGAATACC | |
| | CGGGGGCCAGATGATGAACGCTTTACCTCCCACATGAGGGATCTC | |
| | GTGTTGGGCTCTGCGCCCCCGAGTGCCTTTGGCTCCCTAGCCGCT | |
| | GTCCTCACCCCGTACGTGGGGCGCCGCATACATGAAGTGACTACT | |
| | GCTATGGCGGCCCTCGGGGAAGCAGAAGGCCATCGGCGGGACCGG | |
| | GGGGTCCGCGCCATAAAGAAGGGGAAGGTGCCCCNCCCGCAGGG | |
| | GGCCACCCCACGGGANAAAAGGGGGCCCCAGCGGGTGACNCGCN | |
| | CGCAGATGTGGATNGATTTGATTTCGGCCGGGGTTGACCGAGAGA | |
| | AAATTGATAGGCAGCCCAATGAAGTGCTGTTAACTTTGTGGAGGC | |
| | AATTGTCCCCGGAGCAGCAATTCCGGAAAATGCCCAAGGGGGGGN | |
| | AGGACANTGCTGCGCGACCCAGTCCCGCCCGGGCGCTCCAGCTCA | |
| | AGGACTACTTGCAGNCGGGCGGAGATGTAGAGCCTTTTCTGTTTGA | |
| | TTAGGGAACTGGCCGAGGTGCCCGGCTTGGGGGGACACCGGACGA | |
| | CCGGAGGCCACATGTGGAATTGGCAATCCACTGGTCCCCCACCAA | |
| | TGTACAGCGGGTGCTGGCGCTGGTAGATACTGGCGCAGATTGCAG | |
| | CCTCGTTTATGGGAACCCGGATAAGTTTCCGGGCAAAGCTGCATAC | |
| | ATTGACGGTTATGGAGGCCGGTCAGTGAAAGTGAAACCTGTATCT | |
| | CTGCACCTTGGCATCGGCCGCTTGGCTCCCCGCTTATACACTGTGT | |
| | ATGTCTCTCCCATACCTGAATACATTCTGGGGGTGGACGTTTTA | |
| | CACGGCTTGGNCTTACAAACCACGGCCGGAGAATTCAGACTCCGA | |
| | GTNCGTGTGGTGAAGCCGGTGCTGCGTGGACATACGCATCACCAG | |
| | CCCCAGGTCCTGCCACAACCCCGACGGGTTACTTCCACCCGTCAAT | |
| | ACCGTTTGCCGGGTGGGCATACGGAGATAACTGAGACNATTAAG | |
| | AAGCTGGAGGAGGTGCAGATAGTGCGTGGCACCCACAGCCCCTAC | |
| | AATTCTCCGGTATGGCCAGTCAGAAAGCCTGATGGAACTTGGCGG | |
| | ATGACGGTGGACTATCGGGAACTGAATAAAGTAACACCCCCTTTG | |
| | CATGCAGCTGTGCCGTCTATCACGGATTTGATGGACCGCTTGACG | |
| | ACGGAACTGGGACAGTACCACTATGTGGTGGACTTGGCCAATGCA | |
| | TTCTTCTCCATCGACATTGCTCCAGAGAGCCAGGAACAGTTTGCC | |
| | TTCACGTGGGAGGGGCGACAATGGACTTTCACAGTGTTGCCGCAG | |
| | GGCTATGTGCATAGCCCCACCATATGTCATGGTCTTGTTGCCACG | |
| | GATTTAGCCGCCTGGNAATGTCCAGAAGGGGTCCGCCTATTCCATT | |
| | ATATTGATGATATTATGTTAACCTCTGATTCTCTTGCAGATTTA | |
| | GAAGCGGCGGCGCCCCTCTTGCGGCAACATTTGGCAGCATGCGGT | |
| | TGGGCCGTCAACGAATCCAAGGTCCAAGGGCCTGGATTGTCTGCC | |
| | AAATTCTTGGGAGTTATCTGGTCGGGTAAGACGAAGGCCATACCA | |
| | GAGGCCATCATTGATAAAATTCAGGCATATCCCCGGCCCACCACG | |
| | GTGAGGCAGCTGCAGACTTTTGTGGGCCTCCTGGGATATTGGCGG | |
| | GCATTTGTGCCCCATTTGGCTCAAATGATAAAACCGTTGTATCGG | |
| | TTAACAAAGAAGGGAGCTACCTGGGATTGGGATGATGCGGCTGAG | |
| | ACNGCCTTCCTGGCAGCCAAGCGGGCTATTCAGCAGGCACAAGCC | |
| | CTACGGGTAGTTGACCGGGGCGCCCATTTGAGCTGGATGTGCATGT | |
| | GACCACAGATGGTTTCGGCTGGGGCCTATGGCAGCGCACGGAGC | |
| | GCTTGAGAACGCCAGTAGGCTTTTGGTCCCAACTATGGAAGGGAG | |
| | CTGAGCTCCGGTATTCNTTGATAGAGAAGCAGTTAGCAGCTGCAT | |
| | ATGCCGCCCTTCAGGCTCGTGAGAGCGTGACAGGACGGGCTACAG | |
| | TCGTCGTGCGGACGACTTACCCAATAGCGGGATGGGTGCGTTCAT | |
| | GGGTAACGACCCCCCGGACTGGGACGGCGCAGACATCCACTTTAG | |
| | CAAAGTGGGGCGCCTACTTGGAGCAGCGGAGTACGCTGAGTACAA | |
| | GTCCCTTAGCAGCAGAGTTGCAAGAGGTCTTGGGACCTGTAGTCCT | |
| | AATGCAAGATAAGGCCATGGGGCCTGAGGCACCCCTAGACCCTG | |
| | AGCCTTCACCGTTTAAGGAAGGGCGTCCCCCCATTCCCGATGGGGC | |
| | ATGGTACACAGATGGGTCTAGCCGGGGTGCTACTGCTGCCTGGA | |
| | CCGCTGTCGCAGTCCAGCCTAGTACTGACACCATATGGTTTGANAC | |
| | CGGGTGTGGACAAAGTAGCCAATGGGCTGAACTCAGAGCAGTGT | |
| | GGATGGTGATCACCAAGGAGGTGACACCTATGGTAATCTGCACCG | |
| | ATAGCTGGGCAGTTTATCGAGGCTTAACTTGTGGTTAACTACCTG | |
| | GAAGTTACAGAANTGGCTAGTTGGCCACCGGCCCATNTGGGGCCA | |
| | AGCCATGTGGCAAGACCTATGGGAAATGGGTCATCAGAAAGANGT | |
| | AACTATTTATCATGTGTCAGGCCATATGCCTTTGGCCACCCCCGGC | |
| | AATGATGAGGCAGATGCCTTGGCCAAGGTCCGATGGTTAGAGTC | |
| | GGCACCTACACGAGATGTGGCCTTGTGGCTACACCGGAAACTGGG | |
| | ACATGCGGGGGGTAAACTGATGCAACAGGTCAATAAGCGTTGGGG | |
| | TCTGTCCCTGCCCACGCAAGACATTTGGGAGGCTTGTCAGAAATGC | |
| | CCGGCATGTGCTCAGGCATACCCTAAACGGAGGCAGCTGCCCAG | |
| | TGTTACACAACAAGTGACGATAGGGCGGGTGCCCTTGACCAGGTG | |
| | GCAAGTAGACTACATCGGGCCGCTGCCGAAGTCGCAGGGGTATAC | |
| | GCATGCGCTGACGGCTGTGGACACGGCCACAGGCCTGTTGTTCGC | |
| | CTACCCTTGCAGGGTGGCCGACCAACAGCACACCATCCGGGCCCT | |
| | GCAACACTTATGTGCCCTGTATGGTCGCCCTCTGGCCGTTGAGAGT | |
| | GATAGGGGAACACATTTCACTGGACAACAGGTACAACAGTGGGC | |
| | ACAACAGATGGACATAAAGTGGGGATTCCATGTGCCATACAACCC | |
| | GCAAGCCGCGGGTATGATTGAGCGATATAACGGGCTCTTGAAGAA | |
| | TGGGTTACGCTTGCATGTCACNCCCCCGTCTTTGCGGGGCTGGAGT | |
| | TCCAGGCTGGACCTGGTGCTCCAAACCTTGAATGAACGGCCACG | |
| | GAAAGGCGGCCCGGCCCCGGTGGAGGCNTTGTTACACCGGGCCGC | |
| | CGCCCCCATTCAGTTGCAGATACACACCAAGGATGACCTCCTCCG | |
| | ACCAGGTATGGGGACGAACGGTAACCTGTTGTTGCCTGCCCCAAC | |
| | GCCCCTGAAGGCAGGGGAACAGAAAACCTGGCTTTGGCCATGGAC | |
| | CCTCCAAGCCCCCCACTGCCGGTGGTTGGCCATCGTAGCCCCCTGG | |
| | GGGGAGGGCCTACAGTATGACTTGCATGTCACTCCTTGGGTATT | |
| | CAATGCGTGGCCCCCGCGGTTGACCGTTCGTAGGGGAACGGCCAG | |
| | GGAAGGAACCCTCCTCCGGGGGACATATGTACTGTCTGTGTGGCC | |
| | TATTATGAGCTCCCCCGTGACTTTGGCACGGATACAGGACCCAAA | |
| | GGAACCATGGGGAGCTGAGAAGGTGTGGTACCATCGCCCAGGGCA | |
| | GAAGCCCTTGGCGGCTGCATTGTTATCCAGGGATGAAAGGTTAGC | |
| | CTGTATTTTGCCTGAGGGACGTGATTTACCCCTGTTAGTACCTGT | |
| | GCCTGCTCTGTCGTTTCGGCCGTAGGTTGACATGCTCCAACNGCAT | |
| | TGTGGACTGGGCCCACACCTACGCTGAGGTGACCAATGTTTCCA | |
| | ACTGTTGGATCTGCACCGCCCTTCCAGCAGCAGCTGCGGACGGCTT | |
| | GCCCTGGCACATACATCCAGCNTCTGCGGAGAACTGGACATGGC | |
| | TGGAGACTTGGGGTCCCATGGCCGACGCCTGGAATGCAACGCGGC | |
| | AAGCTTTGGACAGGGGGCGCCGCAAGACCCATGGCANCGCCCGCC | |
| | CCCTGGCTGGCCCGTAGCGTCTATGATGGGTGGGGCTGGCTAGTG | |
| | GGGGAACACGTGGTGCCCCCANCGCAGGTACCACGGTGCATAGAG | |
| | CAACACTGGGGTAACGCCACTGTGGGATGGNTGCCCGCCGCGGCC | |
| | TGTGCAAACATAACACGTGTCACCACACCGAAGGTGTGGTGGAAC | |
| | AAGCGGCCCCACCAAGGCCGGGCCCCGATGGACTTTGTGCCCCCT | |
| | GGGAGTTTATGGGTCTGTGGGGACACAGGGTGGCCNTACCTGCCA | |
| | GCGAACTGGACTGGACGTTGTACCTGGGGGTGGCCTTATGTGCCTG | |
| | CCACTGTTCTCCCCACATTGCCCAGACGCCCGCATAACTGGGAG | |
| | GCGCTACGCTCCCGGTTTTTGCGAGTGCGGCGAGCCCCCTGGTGGT | |
| | TCTACCCCTTGGCAATGACTATCCCTGGAGCGGGTGTCATNACT | |
| | GTAGAAGCGCAAGTTACNGCCCTTGCAGAGCACACCGCTCGGGCC | |
| | CTGAATTACACCCGAGTNGCCCTCCTCCTGTTAACNGATGAGGTT | |
| | GATCAGATCAGAAAGGTGGTGCTGCAAAACCGGATGGCCTTAGAC | |
| | ATAGTAACTGCTGCCCAAGGNGGCACCTGTGCCCTTTTAGGAACA | |
| | CAATGTTGTACCTTTATCCCTGACAATCGGCAGAACATAACAGCAG | |
| | CCCTGCAAGGGGTNTCACGGGAGATTAAGGCGGTCGAGAGCCTT | |
| | ACTGACGACCCCCTGCAGAGATGGTGGGCATCCCTGGGCTCTGGC | |
| | CTACGCTGGGCCCTAATAGTCATAGGTAGCATAGCNGGGATNCTA | |
| | GTAGTGAGCTGTTGCTCTCTGTATTGTTGCTGTGGCCTATGGGTCC | |
| | AGGGCTCCGCCCTATGGGCACGTGTCCCCGCCNAGAGGACNCCC | |
| | TCGGCCTAGGGGGTGGA | |
| 23 | TGTTAAATACAGTGAGTTCCGAGTTTCTCTTCAAAGAACCAGTATG | Consensus sequence |
| | TCAGTATGTTCAGCTCCCCTGTTCTTTGTTCTCCATTTTAAAGT | for transposable |
| | TTAACTTCCGTNNNTTCGNCTCNCCTGCTCCTTGTTCTCCATTNNAA | elements that belong |
| | ANTTTNNCCCGCCAGTTCTAATCAGTAGTTCACATCTGTTCCC | to LTR10A |
| | CTGGTCACCTGCTCCGTCCTGAGTCACCCCTGGTCACCTGCTCTGA | subfamily |
| | CCTGAGTCACCCTTAGTCACCTGTTCTGTAACCGTCCTTCCCGC | |
| | CAAACTACTCACCCCGCCACTCCGGCTCGTACCCCTGCTCTCTTTA | |
| | AAATAGCCAATCGGAATTAGCTTAGACTGTGCGGTCCAACCCTA | |
| | GCCAATAGGGGAACGACACAGCAGTAGGGGCTACCTGCGTCAGGA | |
| | ATAAGAACCCCTTCCCCTCCCTTGTCCAGGTGTGCTCTCGCCATT | |
| | GCTCCATCCGCGAGNCGCACCCTTCTATAGAAGTAAAATTGCCTTG | |
| | CTGAGAAAATTAAATTTATGTTCGAGTGCTATTTCTTTTGCGGC | |
| | ACCGAAACAAGCATTAAATTTATGTTCAAGTGCTATTTCTTTCGCG | |
| | GCACCGAAACTTTACGTATAACA | |
| 24 | TGTAGAAAGTAAAAAGTTTCCTCTTCAAAGTTTCCCTTCTTGTTAA | Consensus sequence |
| | AGAATAAATCATAAGTGTTAGAAATAATAGTTTCTTTTAAAGAC | for transposable |
| | TAACTTCCTTCAAGCCTCCTTGCTTTGTGCTAATAACTCTTTGTTAA | elements that belong |
| | GCCCTATCCTATGTAGCTGTTAGACATAGGGAATAAGTACATT | to LTR10E |
| | CTATGTCCTTGTACTTTAACCAAGATATTTGTGCTGGACGTGCTCA | subfamily |
| | CAGGCACGTCCCAGCTCGCAGCCTATGCCCCTTCCTTATTTGGA | |
| | AATGTTATTGCTTCTCTAAGTCCTTTCGTAAGCAACTTCCTCTTTTC | |
| | CTTTGTTCTCCCTTGCNTTTACCTATTTAGGAAAGTTTTAAGT | |
| | TATTAGCCAATCGGGTNTCAGTTTAGATTGTGAGGTCCGGCTCCAG | |
| | CCAATGGAGACAGGACACAGCAGCAAGGACAAACTGCGTAAGGG | |
| | ATAAANATTGCTTCCCTCCTTTGTTCAGGTGTGCTCTCGCCATTGTT | |
| | CCATCTGCGANGAGCACCCTTTCTGCAGAAAGTAAAGATTGCC | |
| | TTGCTGAGAGAACTTTTTGTCTGAGTGCTGACTTTTCTTTGCGGCAC | |
| | CGAGGAACAAGCATTCTGTTTCTAAATAAACATTTTACTTATA | |
| | ACA | |
| 25 | TGTGGGCGGCAAGCCACCCAGGCGCCGAGGCAAGAGACCGAGGG | Consensus sequence |
| | CACGAGCTGTTCCAGTATAATAAAATATATAAAACAAGAATAGTT | for transposable |
| | ATACCAGATATAGATCTTAGATATGATTATATATGAATATCATTAA | elements that belong |
| | TCATTAGTTTGTAGCAATTACTCTTTATTCCAATATTATAATAAT | to LTR13 subfamily |
| | CCTCGCTCTATAATCATAACCTAGGAAAAACCAGGCCATACAGAG | |
| | ATAGGAGCTGAGGGGACATAGTGAGGAGTGACCAGAAGACAAGA | |
| | GTGCGAGCCTTCTGTTATGCCCGGACAGGGCCACCAGAGGGCTCC | |
| | TTGGTCTAGCGGTAACGCCAGCGTCTGGGAAGACGCCCGTTGCCA | |
| | GGCGGACCGTGGTCTAGCGGTAGCGTNAGTGTCAAGGAAAAACAC | |
| | CCGCTACTTAGCAGACCGGGAAAGGGAGTCTCCCTTTCCCCGGGG | |
| | GAGTTTAGAGAAGACTCTGCTCCTCCACCTCTTGTGGAGGGCCTGA | |
| | CATCAGTCAGGCCCGCCCGCAGTTATCCGGAGGCCTAACCGTCTC | |
| | CCTGTGATGCTGTGCTTCAGTGGTCACGCTCCTAGTCCGCCTTCAT | |
| | GTTCCATCCTGTACACCTGGCTCTGCCTTCTAGATAGCAGTAGC | |
| | AAATTAGTGAAAGTACTAAAAGTCTCTGATATGCAGAAATAATGG | |
| | CGTAAGCTGTCTCTCTCTCTCTCCTCTCTCTCTCTGCCTCGGCTG | |
| | CCAGGCAGGGAAGGGCCCCCTGTCCAGTGGACACGTGACCCACGT | |
| | GACCTTACCTATCATTGGAGATGGCTCACACTCCTTACCCTGCCC | |
| | CTTTGCCTTGTATCCAATAAATANCAGCGCAGCCNGGCATTCGGGG | |
| | CCACTACCGGTCTCCGCGCCTTGGTGGTAGTGGTCCCCCGGGCC | |
| | CAGCTGTCTTTTCTTTTATCTCTTTGTCTTGTGTCTTTATTTCTACAC | |
| | TCTCTCGTCTCCGCACACGGGGAGAAAANCCCACCGACCCTG | |
| | TGGGGCTGGTCCCTACA | |
| 26 | TGTAGTGGATGCTGTGGTGCGCCGCCCAGATCGNNCCCCCCTTCAG | Consensus sequence |
| | GACCGAGGCACTCATTCCCCCAGCTGCCGGGAGTGTTGGCTGCT | for transposable |
| | GACGGCTCNCAGCTGAGTCCCTCTCCGGGAATTGCCCTCGGCCGA | elements that belong |
| | AGGGAGCTGCCTCGCCCAAGGTTACGCCCCCTTCCCGGGGGCAGC | to LTR16 subfamily |
| | CCGCATCCAATGACTGGTCGATGCGGGGGTACAAAGGCCTCAGTC | |
| | CTCAATTCGGGACAACTCTGAAGGGCCATCCCAGCTCCAGAGCTC | |
| | CCCGTGGGATCGGCTGAGGCCTCTGTTGCGACTGCATCGCAGTTCA | |
| | ACTTCTCCCTCTGCCCAATCCTGCTTCCTTCACTCCCTCACAGG | |
| | TGTTGNTCCCGAGAGCACTCCCCAATAAACCTCCTGCACGCAAATC | |
| | TCCGTCTCAGAGTCTGTTTCCCGGGGAACCCA | |
| 27 | TGTGGTGGACGCTGTGATGCGCCGCCCAGATCCCCCTTCAGGANTG | Consensus sequence |
| | AAGGACTTATTCCCCCAGCTGCTGGGAGTGCTGCCGGCAGACAG | for transposable |
| | CCCTCAGCTGTCAGCCCTCTTCGGGGATTGCCTCAGCTGAAGAGAG | elements that belong |
| | CCGCCTCGCCCAAGGTCACGCCCCCTTCCCGGGGCAGCCCGCAT | to LTR16A |
| | CCAATGACTGATCGATGCGGGGGTATAAAGGCCCGGCCCCCTCGC | subfamily |
| | CCCAACTCGGGACAACTCTGAAGGGCCATCCCAGCTCCAGAGCTC | |
| | CCCGTGGGGTCGGCTGAGGCCTTCGTTGGGACTGCATCGCAGCTCA | |
| | ACTTCTCCCTCTGCCCAATCCTGCTTCCTTCCCTTCCCTTCCAC | |
| | AGGTGTTGATCCCAAGAGCACTCCCTAATAAACNTCCTGCACGCTA | |
| | ATCTCCGTCTCAGAGTCTGCTTCCCGGGGAACCCAACCTGCGAC | |
| | A | |
| 28 | TGTAGTAGATGCTGTTGGTGCCCCGCCCAGATCCCCTTTACCGGGC | Consensus sequence |
| | CGGTGCACCCATCCCCCAGCTGCTGTGAGTGTTGGCTGCTAACG | for transposable |
| | GCTCACAGCTGCCCCCTTCTCCGGAGAATTGCCCTCGGCCGACGGG | elements that belong |
| | AGCCGCCTCGCCTGGGAGGTTACCCCCCACCGGGCAGCCCGCAG | to LTR16A1 |
| | CCAATGACTGACTGATACGGGGGTACAAAAGGCCGGCCCCCTTGC | subfamily |
| | CTCAAGGTGGGACAACTCACTCTGTGGTGCAATTCATGCTCCAGA | |
| | GCTCCCCGTGGGATCAGGCTGAGGCTAGACTTCAGCTGAGACCAC | |
| | ATCCTTGCTTAGCTTCTTCCCCTGCCCTATCCTGCTTCCCTCACT | |
| | CCCTTACAGGTTTCTCCTGAGAGCACTCCCTCAATAAATCACTTGC | |
| | ACAAGAATCCCCGTCTCAGGCTCTGCTTCTAGGGAACCCGACCT | |
| | AAGACA | |
| 29 | TGTAGCGGACACTGTCGGTGCCCGCCCAGATCCCCTCGGATCCCTT | Consensus sequence |
| | TTACCGTTTCTGTGCGCCCCTTCCCCAGCTTCTGTGTGCTTTTG | for transposable |
| | CTTCTAACGGCCCGCACCTGCGACTCTTTTCGGAGGACTGCCCTCG | elements that belong |
| | GGCTACTGGAGCCGCTTTGCCCGCATGCAGAGAGCCGGAAGTGC | to LTR16A2 |
| | CTGGGAGTTTATGNCCTCTTTCCCTCCAGGGCGGCCCTTAGCCAAT | subfamily |
| | GACTGACTGGTGCGGGAGTATGAAAGCCCAGCTCCCTTGCCTCG | |
| | AGTCGGGACAAACTCTGAGGCGTAATTTACACTCCAGAGCTCCCCT | |
| | GCGGGATCAGGCTGAAGCTGGGACTTTGCCTGAAATCGCACCCT | |
| | TGCTTGGCTTCTTCCCCTTCCCTGTCCTGCTTCCCCACTCCCTTACC | |
| | GGTTTCTCCTGGGAGCACTTCCTTAATAAATCACTTGCACACG | |
| | AATCCTCGTCTCAGGGTCTGCTTCTGGGGAACCCGACCTAAGACA | |
| 30 | TGTGGCGGCCATGAAAATGCGCCGCTCAGATCTCCTGCTGCGGGG | Consensus sequence |
| | AGCATAGTTGACTGACGGCCCCAGCTGCTGCCCCTCTGGATCCAC | for transposable |
| | CACCGCGTTCGCGCCGAGGCCACGCTTCCCNCGGGCTGCTCCCAG | elements that belong |
| | CCAATGACTGAGCACGGCGGGGGTACTAATGCAGGCCCATTCCTG | to LTR16B |
| | CGAGACGCGGGACTCCTCTAACGGGCGACTTTGGCTCGAGGACTC | subfamily |
| | CCCATCGGCCTGGCCGAAACTTTCTTAGAACTGCGCTGCAGTCTG | |
| | AGACTCTTCCTACCCAATCCTCCTTCCTTCCCCCTCTCCTTCACAGG | |
| | TGTCAGACCTGCATCGCGGTCTGAAGGCTCTCCCTGCCTNCTC | |
| | CTGCTCCCTCCCCTTTATCCTTCACAGGCGTTTCCCCCAATAAATCT | |
| | CTTGCACGTCTAATCCCGTCTTGGCGTCTGCTTCTCGGAGGAC | |
| | CCGAACTAACACA | |
| 31 | TGTGGCGGCCATGGAGGTGCGCCGCTCAGATCTCCCTTCAAGAGA | Consensus sequence |
| | ACCTGCTGCGGGGAGCGTAGTTGGCTGACAGCCTCCAGCTGCCGC | for transposable |
| | ACCTTCGGATCCGCCGCAGCGTTCACGCCGAGGCCACGCTTCCCCC | elements that belong |
| | GGGCTGCTCCCAGCCAATGACTGAGCACGGCGGGGGTACTAGAG | to LTR16B1 |
| | CCGGGCCATTCCTGCCCGACGCGGGACTCCTCTAACGGGCAATCTT | subfamily |
| | TGCTCNGGGACTCCCCATCGGCCTGGCCGAGACTTTCTCAGAGC | |
| | TGCGCTGCAGTCTGAGGCTCTTCCTACCCAATCCTCCTTCCTTCCCT | |
| | CTCTCCTTTCACAGGTGTCAGACCTGCATCGCGGTCTGAAGGC | |
| | TCTCCCTGCCTACTCCTGCTCCCTCTCCCCTTTATCCTTCACAGGCG | |
| | TTTCCCCCAATAAATCTCTTGCACGTCTAATTCCGTCTTGGCG | |
| | TCTGCTTCTCGGAGGACCCGAACTGACACA | |
| 32 | TGTGGCGGCCATGAGAATGCGCCTCTCAGATCTCCNACTGCAGGG | Consensus sequence |
| | AGCGTAATTGACCGANGGCCCCAGCTGCTGCGCTCTGAATCCATC | for transposable |
| | ACCGCGTTTGCGCCGAGGCCACGCTTCCCACGGGCTGCTCCCAGCC | elements that belong |
| | AATGACTGAGCGCGGCAGGGATACTAAGGCAGGCCCGTTCCTGG | to LTR16B2 |
| | GAGACGCGGGACTCCTCTGACGGGCGACTTTGGCTCGAGGACTCC | subfamily |
| | CCGNCGGCCTTGCCGAANCTTCCTTAGAACTGCACNGCAGTCTAA | |
| | GACGCTTCCTACCCAACCTTCCTTCCTTCCCTCTCTCCTTCACNGGG | |
| | GTCAGACCTGCATCGCGGTCTGACGGCTCTCCCAGCCTCCTCC | |
| | GGCTCCCTCCCCATTTTCCCTCACAGGCGTTTCCCCTAATAAATCTC | |
| | TTGCACGTTTAATCCCGTCTTGGCGTCTGCTTCTCGGAGGACC | |
| | CGGACTAACACA | |
| 33 | TGTAGCAGACGCTGTCGGTGCCCCGCCCATATCCCCTCGGCCCTTA | Consensus sequence |
| | CCATTTCAGTGCACGCCGGCCCGACTTCCAACTGCCAGCACCTG | for transposable |
| | CATCTCTTTGCCTGAGGGCTTTCTCTGGCCGCCGGAGCCCGCTCTG | elements that belong |
| | CCCACGCGCANGGCAGGCCGGAAGTGCCGGGGAATTAACGCCCC | to LTR16C |
| | CCGGGAGCAGCCCTCAACCAATGACTGACGGGAGTTGGTGNATAA | subfamily |
| | ATACCCCAGCTCCCTCGCCCCTCGGGTGGGATAACTCTGAGGCGC | |
| | GTGTTCTACACTGTCTCCCAGAGTTCCCCAGCGGGATTGAGCTCCA | |
| | GTTGCCCACAGTGGTAACTTGCTCGATAACGCACCCTTTATTGG | |
| | CTNCCTTCCCTTCCCTGTCTCACTTCCCCACTCCCCTACCGGTGTTT | |
| | CCTGGGATCACCTCCCAAATAAACTACTTGCACTCGAATCCTT | |
| | GTCTCAGGGTCTGCTTCTGGGGGAACCCAAACTAAGACA | |
| 34 | TGTATCGGACACATCTTATGCGCCTCCTCAGATTCTCTCGGCCTCA | Consensus sequence |
| | CCTGTCTCCCGGACCCTCGGCCGCTTGCTCTGCCTCAGCCGCCG | for transposable |
| | CCGCGGCCGACCGACTCCGTGCGGGCCCATGCGGTTCCGCCGCCT | elements that belong |
| | GAGGCCGACCAGCCACATGCCCGGAGTCTCTGGCTCCTCCCGCCA | to LTR16D |
| | CTTCCGGACTCGGATGAAACGCCACACCGCAGGGCGTGGGATCTG | subfamily |
| | GCTTCCCNAGCGGCTGCCGAAGGGGCCGGATGACGCAACCCGGAA | |
| | GTGCGGGGGAGTTAACTCCCCGTGGGGCGAACTTTGACCAATGGG | |
| | AGACAGGAGACGGGAGGGAGCCGGGCAGATAAATTCCCTCTCCTT | |
| | CCCTCCCTCCATGGACTGTTCCGAGGCACGGTTTCTCCGTACAGCC | |
| | TGTCCGGAGACGTCCCGCGTGGCCGAGCGGATGCACCTGCCGAG | |
| | CGACCGGCTGTGTCTCTTCGCGGCTCGTCGTGAAGCNGTGGCCAGC | |
| | NCGGTAACGCATCACCTTGCATTGCTTCCCNTCCTTCCCTGCCT | |
| | CACTTCCCTTTTCCCTCACTCTCGCTGCCCTGGGATTGCACCTCCCA | |
| | AATAAAGCATTAGCACNTAANCTTTGCCTCAGGCTCTGTTTTC | |
| | TAGGGAACCCGGGCTAAGACA | |
| 35 | TGTATCGGACACAAACCTTGTGCGCCTTCTCAGATTCCCTCGACTG | Consensus sequence |
| | CCTCCTTCTTCCGAAGGTGCCGCCTCCTNCCCGGGCCACTCCGC | for transposable |
| | TGCCACCGCCTTTGCTGAGCTCCCTGAGCTGGCTGGAGTCCGAGTT | elements that belong |
| | GAAGCCCCGGGCCTGCATGGCCCTCAGTCCCACACGCCGAGCCC | to LTR16D1 |
| | CATCCGCGCCGAGTCGCCCCTCCACTTCCGGACTTGGATGAAACGC | subfamily |
| | CACACCGCGGGGCATGGGATCTGGCTTCCTGAGCGGCCGCCGAA | |
| | GGGGCCGGATGACGCAACCCGGAAGTGTAGGGGAGTTAGCTCCCC | |
| | GTGGGGTGAACCTTGACCAATGGGAAACGGGAGACGGGAGGGAG | |
| | CCGGGCAGATAAATTCCCCCTCCTTTCTCCCTTCCGTGGACTGCTC | |
| | CGAGGTGTGGTTTCTCCTTGCAGCCCTTCCGGAGAAGTCCCGCGT | |
| | GCCGAGCGAACACGCCTGCCGAGCGACCTGCTGTGTCTCTTCGCG | |
| | GCTCGTCGTGAAGCGGTGGCCAGCGCGGTAACGCATCGCATCGCA | |
| | TTGCTTCGCATCTTTCCTTGCCTCACTTCCCTTTTTCCTCACCCTCAC | |
| | CGCCCTGGGCTTGCACCTCCCAAATAAAGTGTCAGCACTTTA | |
| | ATCCTTGCCTCAGGCTCTGCTTTCTAGAGGACCCGGGCTAAGACA | |
| 36 | TGTATTGGACACAAACCTTGTGCGCCTTCTCAGATTCCCTCGACCG | Consensus sequence |
| | CCTCCTTCTTTCTCTCTCTTGGCCGGCGCCCCACCCGCGCCGGG | for transposable |
| | TCGCCCCTCCACTTTCGGACTTGGATGAAACGCCACACCGCGGGGC | elements that belong |
| | ATGGGATCTGGCTTCCTGAGCGGCCGCCGAAGGGGCCGGATGAC | to LTR16D2 |
| | GCAACCCGGAAGTGCAGGGGAGTTAGCTCCCCGTGGGGTGAACCT | subfamily |
| | TGACCAATGGGAAACGGGAGACGGGAGGGAGCCGGGCAGATAAA | |
| | TTCCCCCTCCTTTCTCTCTTCCGTGGACTACTCCGAGGTGCGGTTCC | |
| | TCCTTGCAACCCTTCCGGAGAAGTCCCGCGTGCCGAGCGAACAC | |
| | GCCTGCTGAGCGACCTGCTGTGTCTCTTCGCGGCTCGTTGTGAAGC | |
| | GGTAGCCAGCGCGGTAACGCATCGCATCGCATTGCTTCGCATCT | |
| | TTCCTTGCCTCACTTCCCTTTTTCCTCACCCTCGCCGCCCTGGGCTT | |
| | GCACCTCCCAAATAAAGTGTTAGCACCTTAATCCTTGCCTCAG | |
| | GCTCTGCTTTCTAGAGGACCCGGGCTAAGACA | |
| 37 | TGTAGCAGACGCCTCTGGTGCCCCGCGTCACATCCCCTCGGCCCAC | Consensus sequence |
| | CTCTGATTTCAGCCGCAGCTGCGGTGGACAGTTCCGTGCGAGCT | for transposable |
| | CAGACTCACCTTTGCTGACAGCGTCCCACCTCAAGCGCGCGCCGTG | elements that belong |
| | CGTCTTTCTGCTTTCTGCCCCAGGGCCTTCTCCGACGCCGCGGG | to LTR16E1 |
| | AGCCCGCTCGGCCCGCGCGCAAGCGCAGCCCGGAAGTGCGGGGG | subfamily |
| | AGTTAACGCCCCCGGGGGCAACCCTCAACCAATGGGGGACGGGAG | |
| | CCGGTGGATAAATGCCCCAGCCTCCCGTCCTTCAGGTGGACAATTC | |
| | TGGGAGGCATTCTGTACGCTTCTCAGGAGGTCCCAGCGGGATCGA | |
| | GCCCCNGTTGCCCACAGCGGCGACCTCGATAACGCACCCTTATATT | |
| | GGCTTTTCCTCCTTCCCTGTCTCACTTCCCCGCTCCCTCACTCC | |
| | TGCTTCCTGGGATCACCTCCCAAATAAACTACCTGCACCCAAGTCC | |
| | TTGTCTCAGGCTCTGCTTTCGGGGGAACCCAAACTAAGACAG | |
| 38 | TGTATCGGACACCTCTTGTGCACCACTTCANATCCTCTCGGCCCCA | Consensus sequence |
| | CCTCTTACTCCAGCCACTGCTGCGGCGACCAGTTCCGCGCAGGC | for transposable |
| | TTCGACCAGCTTCGCGCAGGTGCAACCTGACAGCGCCTCGCCTCAG | elements that belong |
| | GCCCGCGCCGCGTACCTCTCGCTTCCTGCCCCGGGGCTTCTCTG | to LTR16E2 |
| | ACGCCGCGGCGTGGGACGCCTGCGGGAACCCGCTCGGCACTCACG | subfamily |
| | CATGCGCAACCCGGAAGTGCGGGGGAGTTAACGCCCCGTGGGGCA | |
| | ACCCTTGACCAATGGGGGACGGGAGCCGATGGATAAATGCTTCCC | |
| | CCTTTCGTCCCCCGGGCGGACAGTTCTGAGACGCATTTCATANGG | |
| | CTCCTCAGAAGGTCCCGGCGGGATCGAGCACCAGTCGCCCACAGC | |
| | GGTGGCCAACTCGATAACGCATCCTTGTATTGGCTTTCCCTCCTT | |
| | CCCTGTTTCACTCCCCCTGTCCCTCACTCCTGCTCCCTGGGATCACT | |
| | TCCCAAAATAAACTACCTGCACGCAAGCCTTTGTCTCAGGCTC | |
| | TGCTTTCGGGGGAACCCAGGCTAAGACA | |
| 39 | TGTTATAGGAGATAGAAAGAAATTATTTAGGTAGACAGTTAGGGT | Consensus sequence |
| | AAAGNGAGTCCCCGGCAGAAAACTTTCCTTCTAACAAAAAGCAGC | for transposable |
| | TCAGAAATAGCTCCCTTTCTAACCNCACGCAGTTCAAAGAAATCAC | elements that belong |
| | TTCTCTTCTAACAAAGAGCAGCCTGGAAGATCAGGCTGTAAAAC | to LTR19C |
| | ACAGATAAGCAACTCNGGCACAGAAGGAGNGGGGAGTCTCCTGG | subfamily |
| | GTAATCACCAAACTTCACACNCATACGATGGGCCCCAGTAAAAAC | |
| | AGTGGGCCTTAATAAGCACATTCCTTTCCCTTTAGGCACACTAAGA | |
| | TAGGGAAGCTGGAAGCGGACTGGGGGGGGATGCCTGCAGCTGCA | |
| | AGAAGATGCCTGGGAACAGGCACGGAAACTCTCCCTCCCAGATAA | |
| | GCAAGACAAAGCAGCGCGGAGCAGCAGACTAAGAGCCCGCCTGC | |
| | GTGATCAAGGAATGGGGTGGGGGCTGNTAGAAAACTCTGCTCTAT | |
| | GCAGATGGCACACCTGGTCCCAACCGAATCTTCGGGCCCTANGNG | |
| | GATAAGACACCCCCTCCTCACTAGCCCCCTCCTCACTAGCCCATTT | |
| | ATAAAAACCCTGACATTTTTACTGCAGCNCGGCAACCCGTTCGGG | |
| | ACCCCTCTCTGTGACAGAGAGCTGTTCTTTCCTTTCGCCTATTAAAC | |
| | TCCTGCTCCAANCTCACCCTGTGTGTGTGTGTCCGCGNCCTCGA | |
| | TTTCCTTGGCCGTGAGACCAAGAACCTTGGTATTTACCCCAGACAA | |
| | CGAGGCTGCTTCA | |
| 40 | TGTGAAAATAAGTAATTCAAAATCTAAGCTGTTGGAACTTTAAATT | Consensus sequence |
| | ATTTTGAGCCTTAAAGGAATGTGATTATGGGGCCTGAGTCACGT | for transposable |
| | GACAGGCAGCTGTAACCTAGGCAGCTGTAACCTTTGTTTCTCTGAT | elements that belong |
| | TATAGATTANGCCTTCTTCCTTACCTACATTGTTTTGTAAAATG | to LTR24C |
| | TTGTAAATGACTAAAGGGCGCCAGGGAAGACCCCTTCCCTCTTCAC | subfamily |
| | TGTTGATCTTCATTATAGATTAACTTCCCTCTTACCTCTCTCAC | |
| | ACAAAGACTTCATGACTATCACATTGTCTTAAGATGGAATGTTAAA | |
| | TACACTCTTTTAAATTGGAAAGGAAATGAAAACAAGCTGTAAGG | |
| | AAAAGAAAACAAGCTGTACGGAAAAGAAAANAAANCAAACTGTA | |
| | ACTAACTAATTAAATTGTTGTAACTCATAAACCAGCCTTGTATAGA | |
| | AAATGTTATAATCCTGCTAAATTTCTTTGTTTTCTGCCTATATAAGC | |
| | AAGACCTTAACTTTTAACTTCGGAGCACTGACCCCATTTCTCT | |
| | GGAGTCTGTGTTTCCCGGATGGCCATTCCCAGCTTTTCGCTTGAAT | |
| | AAACTCTTTAAAACTGGATTCTGATCCTTTCGATTATTTCAGGT | |
| | TGACA | |
| 41 | TGAAACCGTCCCTATAAACTTTATAAAATTAATCAGGGAAGAAGG | Consensus sequence |
| | GAGGGGGAGAAACGAAAATAAACCAAGCTTGCAGCACATTCAGC | for transposable |
| | ATTAATCATTAGGTCAGCTTGCTCTCTGACCTGCTTCCTCATAGTTG | elements that belong |
| | TTTGGTGCCTATTGCCCCAGAATCACGTAGACCCTGTTACAAGA | to LTR26 subfamily |
| | TTATAGTTCCCCTTAACTGCTCTATAGATAACAACTTGAACATTAT | |
| | GAAACGTTAAGTTTTCCCTTTGAGATATTCTTTCAGGTCCTGCA | |
| | TACCGGTGAAACTACTGACANNCGNCGTCAGCTGGTCTGAAGGAC | |
| | CCCACGAGGAGCTGACTCACCAAAGAATGCAGTTTCCACATCCTG | |
| | ATGATTTCATCCCCCTTACCCCGACCAATCAACGACCCCAATTTTC | |
| | CAGCCCCTCGCCCTCCACGATCCCCTTAAAAACCCCAGCCCAGA | |
| | ACTCCTCGGGGAGATGGATTTGAGGGTCTCCTCCCATCTCCTCGCT | |
| | CGGCGCCCTGCGATCATTAAACTCTTTCTCTGCTGCAAACCCTG | |
| | CTGTCTCAGTGTAATTGGTCTGTTACTGCGCAGCGGGCATACGAAC | |
| | CTGTTGGTCCTATAACA | |
| 42 | TGTGCTGGATATTTTCCGTTTGCCCCTCCAGATCCACTCTCCACCCT | Consensus sequence |
| | TCTCCACCCTGCTCTGTGCCCCGGGAGGCTGACCTCTATGGAC | for transposable |
| | TGCATCAACGGGCTCCCTTGCCCTCTGGCTTCCGGTTGGGTTCGGC | elements that belong |
| | CAATGGGAGGCACCGGCAGGAGATCGGAGGGCGGGAGGAGAGTG | to LTR33 subfamily |
| | AGGTCGGGGTATTTATTCCCCCGGCTCCCTCCCTGCCGGGCCGCGG | |
| | NTTGGCAGTGGCTGCGTTCCTCTACCGAAGGCCACAGCTCCTGT | |
| | CGGGCGGCCCTCTCCTACAGCTACAGCTCTCTCCGGGTTCCGGTAA | |
| | CCGCTCCCTCCCCTTGCCCCTTCAGGCCTAGGGGTGGTAACGGC | |
| | TCCCCGCTGTTGCTAGCCCCGGGGTGCTTCACCATCCCTTGTTGGTT | |
| | TCCCTTAACCCTGCCCACACCTTTGTAAATAGTCCCTTCATTA | |
| | AACTCTCCTCAATTACCCCCGTTTGAGTGTGCCATCTGTTTCCTGCC | |
| | GGGACCCTGACTGATACA | |
| 43 | TGTTGGGAGACAATCCTCCATGGGCCCCTNGCGCTCCTGCACGTCT | Consensus sequence |
| | TGCTGGGTATGCCAAGAATGCAAGGCCCTGACCGCTCTTTACCT | for transposable |
| | CGGGCCATTTCTCAGGGTTGTGTTTGCAGCGAGCAACCTTGAGGGA | elements that belong |
| | TGAGGTAATGTCTCCCTCCGGGACAAAGAGCAGGCTTGCTTACT | to LTR40c |
| | GCTTGCTATAAAAGCGGTGGATTCCCCAAGCTCAGTGTTCCTCNGC | subfamily |
| | TGTAACGCAAACCCACTGCGTGCGCAGCATCCATCTGGGCCCTC | |
| | CGCGTCGCCCCCGTGGGACTTGGGGGGNCANGGGGAACCGACGCA | |
| | AACATGCTGATGCTCATGCTGCTTGCTGTGCCGTGAGTAATAAAG | |
| | TCCTTTGTCTCTGACCCAGGAGTCTCGTGTCTTCTGCCAGCATCCAT | |
| | GAAACAGTAACAGGCTAACTTATTAGCTTGTAAGTAGGGTAAA | |
| | ATCCCAGACCTGACA | |
| 44 | GGTGTGCTTCTCTCACTTGGCAGGCTCCTGCAGCCAGGCACGGCTT | Consensus sequence |
| | CTCCAGCGCCCGGCTCCTGCAGCGCGCGCAGCTTCTCCAGCGCC | for transposable |
| | CGGCTCCTGCAGCATGGCACTTCTCAGCATGCAGCTCCGCAGCAGC | elements that belong |
| | AGTGCCAGCAGCTTCCCCCGGCACCCTCCTCGGGCGGTTTTGTA | to LTR41 subfamily |
| | GCGGAGTGCCTCCGGTGAGACACCTCCCCGTGAACAGCTTTCCCCG | |
| | GCACCCTAGAGGGCGGATTTCCGGCAAGTTCCAGAGGGCGGATT | |
| | TCCAGCAAGTTCCGCCGGCGCGGCACCACAGCGACTTCTCTGCCAT | |
| | CCAGTGAGCCACGGCCGTGCCCTCTCCAACAAGGTCTGGATCTC | |
| | AGCCCTGGGGGGAGGGGGCTCTTCCTTGGGCGCTCTATCTCAGCCC | |
| | TAGGGGTAGTGGCTGCTCCTTATATCTGCTATTCCTATATTCTT | |
| | TAGAGTTCTCTTTACTTCTTACTAGCCAATCCCTCGTTACTCCAATC | |
| | CCCTGTTATAGTTAATAATTCTTTATATTAAACTTTCCCTGTT | |
| | CAAATTACTGTGTGGTTTCTNTCTCCTGATTGGACCCNGACTGATA | |
| | CA | |
| 45 | TGTGCCAGTTATCAATTTATTGCCTCTCAGCTCCAAATTCACCCTTC | Consensus sequence |
| | AATACCTGCTCTGCGATAATGGACTGGACTCTTTAAGCATTTC | for transposable |
| | TCCTTTACAGTGAGCACGATGTTAAGCTTTNTCAGTAGAGGGCGCT | elements that belong |
| | GGAGGGACATTGCAGGAGGAAGGGGGCTTCTCTTCCTGGTTCCG | to LTR41B |
| | GTGTGCTGCGTTTTGCTTTTTCTTGCTCCTGCTGCACGGTCGGTCAG | subfamily |
| | CGGTGCGGGTGTGTGGGGACATCCGGTGGTGCTCTGCCCCAGC | |
| | CGCGCGCCCAGAGCGCGCAGTCCCTCGGCGACCTCGCAGCCCCGG | |
| | CCTGGGCCCGGTGACCACCTTCCCGCGGCCCTCCCGACGCGGACA | |
| | CCGCGTGCTCCAGGCCTCGCGCCCGCAGCGGCGCCCCGACTCCCTC | |
| | TGCGCGCCCGCCCACCAGCCTCGGCTCGCCTGCACCCCGGAGGG | |
| | TTGCTTCCTGCTTGCCCAGCGACTGCGGACCAGCTCTGGCCCGGGC | |
| | AANCCAGCGAACTTCTCTGCCATCCAGTGGGCTGCAACCACACC | |
| | TTCTCCAACGAGGTCTGAACCCCAGCCTTGGGGAGGGGGCCCCCC | |
| | TTCCAAGTTTGTCCTTCCTTGGGTACTCTCCCTCAGCCCTAGGGT | |
| | ACCCTTTAGAGTTCTCTTTACATCTTATAGTTACTCTCCTATCATAG | |
| | CTTAATAATTCTTTATATTAAACTTCCCCTGTTTAAATTACTG | |
| | TGTGGTTTCTGTCTCCTGATTGGACCCAGACTGATACA | |
| 46 | TGATAACCTACAGGTCACATTTGGCAGGCTTCCAAATTAACCCGCC | Consensus sequence |
| | TNGGGGAGGTCTTGTGATTCATGGCNACATCCTGTCCCTGAGTA | for transposable |
| | AAGAATCTTGTGAGTCCCTCAAATCTTATCGTGAGTTCCTCAAACT | elements that belong |
| | GTTGACGTACTGATTAATACGTAACCTACTGACACTGAAAAGGA | to LTR44 subfamily |
| | CGCTGATTTGTTTCTGAATCATGAAGTTTTGCTGATTTNTTTCTGAA | |
| | TCATGAAGTTTTACTGATTGTCTTGCACGTAGACATTTTAGCC | |
| | TGTATGTTGCAATCTGTAGCCAATGATTGTAACCTCTGTATTGTAC | |
| | CCTCCAATGAAAAAGGACAACTCCGATATGAGGAGTCCCCCTCC | |
| | CTTCTCCTAAACTTTCTTATAAAAGCCTTCCAACTTGTAACAGACT | |
| | CTGGAACACGCCCAACTTTGTTGGTGTGTCTTCCCGGGTCGATC | |
| | CTCACATTTGGCTTCCAATAAACCTTTATCAAATTATTTCTGCCTCA | |
| | ACAGCCTTAATTTCGGTCGACA | |
| 47 | TGTGGGGACAAGAGTGACTTTATTTTAAATGCTAATCCGCCATGTA | Consensus sequence |
| | ACTTCTGACTAACCCCGAGTCCGGGAATGCCTCCAAAATGTCTA | for transposable |
| | GTTGATGTATTACTCTTTATGTAGGAACACCTATTCACTGTAAGTTT | elements that belong |
| | CCTCCAAAACAACCCTTGATGCTGTTGCAGAAATCATAGGCTG | to LTR47B4 |
| | TGACGCCCGTAGCCACCTACACATTCCTTCCAGAGCACGTATACTT | subfamily |
| | TTTCCCCAAGATATAAGCCCTGGGTCTGGGGGGTTGCGGTGCGG | |
| | AGATCTACCTGTCTTGCGGCCGCCCAAGACCACGCTTCTGTCTGTA | |
| | AGTTCCCCTAATAAATCACCCNANACCGACAAACTGGATTTGTC | |
| | TGCCTCCTTCTTTGGTTTCTCGGCTCCTTCGGCATTTGGGGGTCGCT | |
| | TTGCATATACGGCCCTTTCACGGAACA | |
| 48 | TGTGCCGGTTATTAAGCTATTGTCTCTCAGCTCCAAACCCACCCTT | Consensus sequence |
| | CTATACTCTGCTTTGTGATGCTGGGGCTGGGACTCGNNNNTGCA | for transposable |
| | AACCACATTTCTGCTTTGCCAGCTGGCTCTCCCTGTTAGGCTCTGCC | elements that belong |
| | AATAGGGGGCGCTAGAGGGAGACTGCAAGGGGCTGGAGGGGAG | to LTR50 subfamily |
| | GAAGAAGGGACTTGCTCCTTCCTGTCTGCTNCTGTTTCCTGTCTGCT | |
| | TCCTGTTCCTGTCAGCGTCACCCCAGCAACGCTTCTTCACCCT | |
| | GGCAGCAGINGCTTCCAGTTNNTTCCAGCAGCAGCAGTTGANTCC | |
| | AGTTTGCGNNGTTCCAGTTTCCANCANTTTTTCCCAACACTCNCA | |
| | GAACCAGCCTCATCGCGCCCCCTCNNNNNCCTCAGAGACACCAGC | |
| | ACCAGCCGGGCAGCGCCCCCTCCTCAGAGGTCTGGGTCCCAGCTC | |
| | CGCGGGGCCCCTCCTCCGAGCTCAGAGACACCAGCACCAGCCGGG | |
| | CAGCGCCCCCTCCTCAGAGGTCTGAGNTCCCAGCTCCGCGGGGCC | |
| | CCTCCTCCAAGCTTCTAAGTTTTAATAATTCCAACCTCTTCCCTTTT | |
| | GTTCCCCCAGCCCTAGGGGNCGNTGGTAGCTGCTTCCTGCAGT | |
| | TGCTACCTCCGTGATACCTTAGTGTTCTCTTTTTGCCTTTTCAGTTC | |
| | TCTAATNNACAACTTTATACCTAGTTAACCCGNNTTAACAATT | |
| | CTTTATATTAAATTCTCTCTGTTAAAATAACTGGTGTGGTTTCTGTC | |
| | TTCTCCTGACTGGACCCTGACTGATACA | |
| 49 | TGTAACAGGATGTTAAANTGGAAGTTTCGGCTGAGGCACCAAGAT | Consensus sequence |
| | ACAATAGATACCANATTCCAAAGTGAGGTGCCAGACCACNATGCA | for transposable |
| | GANTTTCAAAGGAGGTGGCCGAAAACCGGTTNNACGACTCTTAAA | elements that belong |
| | CCCCTCACCCACATCGAGGGTATAAAAGGGTCGGGANGTTGGAGA | to LTR78 subfamily |
| | TGAGGGGAGATTTGTGAAGAGGATTTCTTTGGAGAGAGCTGTTGG | |
| | TGTGCTGTGAGTCCCACCCCACCCCCAAGGAGAGAGGAGGGGGGG | |
| | TGCTCCCCCTTCACCTCAAGCCNAGNGAGAGGGGCTTCAACGGGA | |
| | CCACTCGGAGAGCTTGATATGTGTCCCCTGCAGTTTGGGGGACAC | |
| | AGTGGACTGGTGTCTGACTCNCGCCTGGGAAATCTAAAGGGCGAG | |
| | AGACGGGCTGGCCAGCTGCTCTGACGGCGGAGCAAAGGAGAGGT | |
| | GGCTGCGCTGGGATCGGCCTGCACTCCCAGAGTTTGTCGGGGCAA | |
| | AGGATGCGTGAGTGTTTCCCGTGGACCAGATGTGGGCCACGCGNG | |
| | AGAGAGAGCCGGCGTGGGGTCGTCTTAGGTCCTGTCCAAGAGGGC | |
| | CGCCTGGAGGGGCGAAGGGACCTCAGCAGAGAGAAGCTGGAGGT | |
| | ACCGCCGGATTCCCAGGGGCTGAGGAAGGAGTAAGCGACCAGCCG | |
| | GAAGAGAGATGCATTCGCCCGCGTCAAGGGAACTGCAGGTGAGAG | |
| | ATCCCCAGCGATGGGGGGGGGGATCTCCGAAGAACCCACGAAAGC | |
| | GCCCACGAGAGAAAGAGTCAGCTTTAAACACCTGCCAGGCCCAGA | |
| | GAGCGCGAAGCCAGCTCACAGACAGTACCAGTCAAGTAAGAACTT | |
| | TCCTGCTCCCCTTNCCTCTCCTCCCTCCCCNCGCTCCAACCCTGGAG | |
| | GGGTCAGAAACCGCGGTTAGCAAGTGGGGGAGGAGGAGNAGGGA | |
| | AAGAGAAAAAAGAAGCCACCACACCCCCTTCCCCGGCTGCAGGCT | |
| | TCCNGCCTGCAGCAGGCCCGAGCTGGGGGAGGGGAGAAGCTTTAA | |
| | CTTTAAATCAAGTTCGGAGTTTTGATTATTACATGGGACTGGACAT | |
| | TTTAATTACTGAATTGAGACTGTGTTTTGTGACTTAAAGTGACCGT | |
| | AGGACTTTTTATTACCTAAGAGTGACCAGAAAAGTCATGGGACC | |
| | TGCCCGAGTTTTCATCCAGGGGCAGGGGAAGAACTANCCCCACTG | |
| | AATAAATTTAAAGGGACAGTGGGAGACAAAAATAAAGTTGCTTTN | |
| | TGATTACATCCCATGAGTCNTGCTTGTTCAACGTACCGGTTACA | |
| 50 | TGAAACCGCCTTTGCAAAGATTATGACAGTGAGAGAAATCTGACA | Consensus sequence |
| | TGGCTGACTCCATCTTGCTTCTAGCCTCACAGGCTGGCTGTCCTC | for transposable |
| | GCTCATTCCTGGGCGTAGGCCAAGCTAACCNTGGGAGGAATTTAG | elements that belong |
| | TTTATAGTTTAACTTTGAAGCAAGGATGATAATAGTCCCTCCCTA | to LTR8B subfamily |
| | AAACTAACCCCCTCCTTGCTCGGGGACCGAAACCGCCTTTGTAAGA | Consensus sequence |
| | CTAATGAAAGGCCACAAGATTAGGATTATGGGAGGGGCCTGAAT | |
| | TCTGCTAAGATGTAGGCATAGTTAAACGATAACCAGCCATTGTNCC | |
| | CGNNNCGNNTTTCTATAATCCCTTACTGCTCAGGAGTCATGTGG | |
| | CCAGAGGTCACAAGATTTGTGACTTCCCCAATTGCTCCTATAGATA | |
| | ACATCACTATTGTAGAACCTAAGATTGGTCTTTTGAGATGTTTT | |
| | TCAGACTTTTGCATTCTGGCGACCGACTGACCCCACCCGGACCCGT | |
| | GACTCATGACTCAACCGGTCCTGTGGCCGNNNNCGNNNCCCACC | |
| | CAGAGGCGGACTCAGCGCACGAGGACCCGTTTTCCACACCCCTAT | |
| | GATTTNCATCCCCAACCAATCAGCAGCACCCATTCCCTAGCCCCC | |
| | TGCCCACCAAATTATCCNTGAAAAACCCTAGCCTCCGAGCCTTCGG | |
| | GGAGACTGATTTGAGTAATAANCTCCAGTCCTCCCGCNTGGCCG | |
| | GCCTCGCGTTAATTAAACTCTTTCTCTACTGCAATACCGCGGTCTC | |
| | AGTGAATTGGTTTTGTCTGTGCAGCGGGCAGGAAGAACCCGTCG | |
| | GGCGATTACA | |
| 51 | TGTTAGAAATTTGATCGGGGATTTCCTGTGCAGTCTGGAGCTGAGA | for transposable |
| | ANAAGGAGATCATGACTTGGGCTTTGCAGCCCAAATATCTTTCC | elements that belong |
| | TTCCCCATCCCCCAAAATACACCCAATCAAATAAGGTTAGGATGAT | to LTR90A |
| | GGTTACCAGGCAGCTTGCAAAAGCAAGGGCATTCCACCCTCCGA | subfamily |
| | ATTTCTGGTCAGAGANAGTANATATGTAAAACAGAATATGCTCCA | |
| | TTGTTAAGCAAACACCCTNTTTATTGATAGTGGGTCAACAAGAGC | |
| | ATAGGCAGGTTTAGCTATACAGACAACTAACAGAATTTGAGATGC | |
| | AGAATATTAAAGCTAAGAATCCAAGATCAATCATTCAAAATACAA | |
| | GTAGATACGCAGAGAAAATATCACAGCATAAGTAGCAGTNCTAAA | |
| | ACGCAAAGGGCTAGAGTCCTGCNAAGGCCTTTCCTACGCAGAAGN | |
| | GCCAAAGCGGAGATTCAGGATCAACGCTGTTAAAATGCAAAATAT | |
| | AATCAGGAGCAAGCNCTANCGAATCCTGCAAAGGCCTTTCTTAAT | |
| | TAAGCANTTCTGANATACATNCAACTCCTGGTTATAGAAGGNNNN | |
| | NNNNNNNNNNNNNNNNNNNNNNNNNNNNCCAAGGGAAAAGCGG | |
| | NGAGAAAGCAGAGAGAAAGAGNAGCCGGAAAATNCCAGCTCCTT | |
| | AAAAGCTCTAAACAAGGGGCGGAGACTCGCTCCTCCCTGGCCAAT | |
| | CCTTGATGGCTCCGGAGAGGGCGCCCTGATTAATACTTTGTTACGT | |
| | CATTAAAAGGGGTCAGCTTAAGGTAAGGTAATGGAGNTCCCATGA | |
| | CCTATTGACCTTGTTACGTCATTAGAGAGGCCANCTTTGGCCCAGA | |
| | AAATGGCGGATTCCTTCCCTGTGATTCACACATTACCCAGAAGCCT | |
| | CTGGCTTCCTGGAATTCTTAATAAAAATTCCAAATCTCTCGGCAAT | |
| | AACTTTCAAACACCACAATTTTCGCTAACA | |
| 52 | ACATTTTCTGGCGACGAGGATGGGATCCTGTTAAAAAGAAAGCTG | Consensus sequence |
| | GACAACAGCAGGCAGTGTAAATCCACTCTCAAGTACTACTGGTAA | for transposable |
| | GCAATTGAATACGGCCTCCACCCTGGAACTTGAGCAGACCCTGTG | elements that belong |
| | CTTAAAATGGCTGCTTTGGAGCATATTGAAGAGTTTGATGTCAGT | to Mamgypsy2-I |
| | CANCCTGCTTCCTGGGATTCATATGCTGAGAGGCTTTCATTCTATC | subfamily |
| | TTGAAGCAAACAAAATAGAGGGCCCTGAACAGAAGCATGCTGTG | |
| | CTCCTGACNGTCTGTGGGGCCCAAACATNTGCCATAATAAGGTCTC | |
| | TCACCGTCCCTGCTTCACCTAAGACTAGGACATTTGATGAACTT | |
| | GTGGGATTATTGAAGGCGCACTTCTCACCAGCACCATCAGTCATTG | |
| | TTCAGAGATTTAAATTTCACAAATGGAATCAACAGCCTGGTGAG | |
| | GGCATTGCTGCTTATATTGCAGAGCTGCGCCATCTGTCTCAACACT | |
| | GCAATTTTGGGGATTTCTTAGATGACATGTTGCGGGATCGCCTT | |
| | GTATGCGGAGTTCACGATGAAGCCCTCCAACGGCGCCTCTTGACTG | |
| | AACCAAATTTGACATTTATAATGGCCCAAGAAAAGGCCCTAGCC | |
| | AGTGAGACTGCATCACTACATGTCACAGAAATTAAAGGAGCAGCT | |
| | TCTAATATAAATGCTGTCCATAACACGCAGGCAGAGAGCTCTTGC | |
| | CAACAGGGTCAGCAGAACACAGGAAATGCNCGTTCCAGAATATCT | |
| | GCAGATCAGTTCAGACATACCAACCCCTGCTCAGGCTGCGGAGGG | |
| | TTACACCAACGCTCTGCTTGCAGATTCAAGGATGCGGAGTGCAGA | |
| | TTCTGCAAGAGGAAGGGCCACCTTGAGAGAGTATGCCGTTCAAAA | |
| | TCCATTAGAATGAAGTCCAGGAACTGTCGTAGCAGTTCACATCACC | |
| | TGGCATCAGGTGATCTTGATTATTCACATGACAGTGAAGCCCAG | |
| | GAATTGTATCATATTCAGAGGGTCCAGGGCAAGATGCCTCAGGAT | |
| | ACTGCAGTATTTGCAACAGTACTCATAAATGGCCATGAGTGCAAG | |
| | ATGGAAGTCGACTCCGGTTGTGGTATATCAATAATCGGTAAAGAT | |
| | ACCTTCCACAATATTTTCTCACAAGGCCCTTTGCCAAAGTTGTTG | |
| | AGGCCACCATGCATTCTTCAAGATTACAATGATCACACAGTTGATG | |
| | TTGCTGGAAGCTGTGTTGTGAATGTGCAGCGTGGCTCCTTCACG | |
| | GAGAAGCTTCCACTTATCATCGCAAGGGGGCAGCGCAAAAGCCTC | |
| | TTGGGAAGGAACTGGTTCACACCACTAGGAATCAGTGTCCAAGGC | |
| | ATTCATACCATTAGAACAGCCCGACTACAGGAAGTTGTCAACAGA | |
| | TTTCCCAAGGTATTCTCCGAGGAACTGGGAGCTTACAAGAGGGAG | |
| | CCAGTGTCATTCCATCTGGATCCAGCCATTGCACCAATTCACTTGA | |
| | AAGCTAGAAATGTACCTTTTGCTCTAAGGAAAAAAANTGAAGAA | |
| | GAGCTTGAGCATCTAATCAAACAAGGTGCCCTCGAACCAGTTACA | |
| | CATACCCGGTGGGCTATACCAATTGCGCCGGTACTAAAGCCTAAT | |
| | GGTAAGGTGAGACTTTGTGGAGACTACAAGTGCACTGTGAATAAG | |
| | GCACTTAAGCAGCACCCATATCCAGTGCCTGCAGTCAATCAACTC | |
| | CTTGCTGTTCTTGCTGGGGAAAAGGTCTTTGCGAAACTTGACCTTG | |
| | CCCAAGCTTATCAACAGCTGATCGTCAGTGATGCTACTGCAGAC | |
| | GCCCAAACAATCATCACTCATCGAGGGGCATTCCGAGTTAAGCGC | |
| | CTTCAGTTCGGAATTTCTGTTGCCCCTGGAATTTTCCAGTGCTTC | |
| | ATGGAGACCCTGCTTTCTAGGATTCCAGGTGTGGTACCATATTTCG | |
| | ATGATGTCCTGATCATGGGATCAACAGAGGATGAACTCGCGGAA | |
| | AGACTTCAGGAAGTCCTACATCGTTTCGATACATCAGGTATCCGAG | |
| | TGAAAAAGGAGAAATGTGAAATCGGAACTTCCAGTGTCACCTTC | |
| | TTAGGCTACCGCATCAATGCTGCTGGTATTCACCCCACTCAAGATA | |
| | AGGTACGTGCAATCCATGACGCGCCTACTCCAAAAACAAAGCAA | |
| | GAGCTTCAAGCCTTCCTGGGGCTACTCAATTTCTACCATGTTTTTCT | |
| | TCCACAAAAGGCAACCATTGCTGAGCCACTTCATCGCCTACTG | |
| | GGAAAAGGCGTTCCGTGGCGTTGGACTCGCCAGCATGAGGACGCT | |
| | TTCCGGAATGTCAAGCAGTTATTGACTTCCGAGTCTGTGCTAGTA | |
| | CATTACGATGAACATAAACCACTGTTTCTTACCACTGACGCTTCAC | |
| | CTTACGGTGCTGGAGCTGTGCTTAGCCACAAAATGCCAGATGGC | |
| | ACTGAGGCACCAATTGCTTACTACTCAAGAACAATGTCATCTACAG | |
| | AATGGAACTACGCACAAATTGACAAAGAAGCCCTTGCTGTTATC | |
| | GCGGGAGTCAAGAAGTTCCACAACTACCTCTACGGCCGTGCCTTTA | |
| | CAATTTGTACTGATCACAAGCCTCTTTGGGGACTCTTTTCTAAG | |
| | GATAAGCCTACGCCCTTAATTATGTCACCACGTATGCAGCGCTGGA | |
| | GTCTTTTGCTCAGTGCTTATGACTACGCGCTTGTCTACAAGCCT | |
| | GGAAGAGCAATATCGAATGCTGATGCCCTGAGCCGTCTGCCACTG | |
| | CAGATCCCAGATTATGTTGTGCCACTTCCACTGGAAGTTTTGATG | |
| | TTAGAAGACCTCCCTGACCCACCTCTTCAGGCTGATCAAATCGCAC | |
| | GATTAACTGCGTGGGATCCCAGATTGGTCCGTGTCTTAAACTGG | |
| | GTGTGGAGGGGGTGTCCAGTAGAGAAACTGCCTGAGGAATTCAAG | |
| | CCATTCACAAGCCATCAACATGAGTTATCTGCCCATAAAGGATGC | |
| | CTTTTATGGGGAAATCGCATTGTTATTCCAGAAGGCGGTCATCACG | |
| | TTATCTTGGCCACACTACATGCAGCACATCCGGGAATTGTTCGC | |
| | ATGAAAGCATTGGCTAGAAGCTATGTCTGGTGGCCTGGAATTGATT | |
| | CTGATATTGAAAAGACTGTAAAGGAGTGTAGCACATGCCAGGCT | |
| | ACTCGGCATAACCCGCCAAAGGCACCTACTTTTCCATGGAAAGTG | |
| | ACCAAGAAGCCTTGGTCAAGAATCCATATTGATTTTGCAGGACCA | |
| | TTCCAGGGGAAGAACTTCTTGATTGTGGTAGACTCCTTCTCAAAAT | |
| | GGCTCGAAGTGCTCCTAGTTCCATCTCAAACCTCTGCTGCAACA | |
| | ATCACAGCTCTGCGCCAGTTATTTGCAACACACGGATTGCCAGACA | |
| | CTATCGTTTCTGACAATGGGTCTGCATTTACCTCAGTTGAGTTT | |
| | AAGGAATTTGTGGACAGGAACCTTATCCGAGCTGTCACCATCGCG | |
| | CCTCATCACCCTCAAGCAAATGGTCAGGCTGAAAGGATGGTGCAG | |
| | ACTACTAAGGATGCCCTGAAGCGGATTGTTGGAGGAGATTGGCCA | |
| | ACAAGACTTGCCAGGTTTCTCATCGCTCAACATGTCACTCCATGT | |
| | TCAACAACAGGAGTCAGTCCTGCTGAATTACTAATGGGGCGACGT | |
| | CTCAAGACTTGCCTCGACCGTCTCCATCCAGACTTAGATGAGGAC | |
| | TTACAAGACAAACAAGAATGTATTCTGGATGCTGCTCTGTCTGCAC | |
| | CTACTCTTCGCTCATTTTTACCACAAGAGTTAGTCTTTGCTAGG | |
| | AACTATGGAGCTGGACCAAGGTGGGTTCCTGCCACTATAATTGAG | |
| | GTCACGGGGCCTGTGTCATACAAGGTCCAGAGTGCAGATGGGCCG | |
| | GTGTGGCATCGCCGTGTTGATCAACTGCGAAGGCGTTCCTCCCAGG | |
| | GTGACTTGACAACAGCAGAACCATTAGAAGCATCTCCTCTAGAG | |
| | CCATCTGGCGTTGATTTTTCAGCTATACCTCCTGAGGACTCACCAG | |
| | AGCCACAAAGTGAAGGAAGCAATAATTCTTCTCTGGCCACCGCA | |
| | ACTGCTGAAGTGGAGTCACCTGGTATTTCTACACCCACTAGTATAA | |
| | CACAGAGACAGCCTCGCAAAAGGGTACCCCCTTGCTGGTTAAAA | |
| | GATTATGTTAGCTAAGGGGGAAGGGG | |
| 53 | TGTTGTCAGTACTTGGGGGCTACAGTGAGGCGTCCCCCAAGAACTT | Consensus sequence |
| | GGAAATAAGGGAAATAAGATTGCCCTCTGAGAGTTAAACTATAA | for transposable |
| | AATCAACACATAATGGATCAGAGCAGAGATATGCAAAGTCACCTT | elements that belong |
| | AAAGAGACAGAATGGCTCCCAGGCCAGCAAGACTTGACTGTAACC | to MamRep605 |
| | TGATTGAATGTATTAACATATCTAAAGAAAGAATGTGTCAATCAG | subfamily |
| | AAGGGAGGTTGGTCAGCCATGGAAGCATAGAAAAAGGAGAGTCA | |
| | CAGAGGCAAACGTTGGCAGTGCCCATCCAAGCACTGTCCAGGGGA | |
| | GAGGGAGCCTTGGCCAACGGACTTCGAGTATTCCGGTCCCATTGG | |
| | CAAGGCCATCCCTACTGTGGAGATGGATGCATCAGGCTATCTTAGC | |
| | CTGATGCCATTAAACATTCGAATCATNTAACTAACTCCCCTTGTT | |
| | CTGATTCCATGCTCACCAAGCAACCTTCTGATTATAATTTGGCTTC | |
| | CCATGTAGGTCTGCCTACAGGGAGAGAATGCGCTGGTCATGCNG | |
| | ACNCGAGTATTCTGGTCCNCGTGACAGTTATATTTCTCATTGTAAA | |
| | TTAATAAATTGGCATTCTGGTTTTATACATCAGTCTCGTGAGCT | |
| | TGGTTAATGTGAGTCCGCCCCTGAGAGATGGATCCGCTCTCGGTGT | |
| | GCTGACAAAATCAGGTTTCTGAGCCTGCAAGGCTGAGGGCCAGA | |
| | CCCTGGACCGTGGTAAATTCACCAAGGTAATGCCACCCTGTGAGG | |
| | CCTCTTGGGAGACAGCCAGCCGGCCTTTCCCCTGGTGACACTCCA | |
| | NTCNCCACGATGTTGCCATTGGCATTTTAAATTCTTACA | |
| 54 | TGTGATATTGTGATATAATAAGAAATATATATTTGGTCTCTGCCCC | Consensus sequence |
| | CGGTTCCTGGCACAGAGCTCCTAAAACCCTTGTAATTTCCTGAG | for transposable |
| | CGATAGGGGTGCTAGGAGCATCTTTTGTTCTAATATTTGGTCTTTG | elements that belong |
| | NCCCCGGTTCCTGACACAGAGCTCCTAAAACCCTTGGAATTTCC | to MER21C |
| | TGGGTGATAGGAGCGTCTTTTGTTCTAATGAGGTGACTCTTGGCTG | subfamily |
| | GGGGCTCCTGGATAGCCTCAGGATGGGGGCTGGTCGCCAGAGGA | |
| | ACCAACCATGTGATTAGAGGGTTGGAACTTTCAGCCCCACCCCCG | |
| | ACCTCCGGGGAGGGGAGAGGGGCTGGAGATTGAGTTAATCACCAA | |
| | TGGCCAATGATTTAATCAATCATGCCTACGTAATGAAGCCTCCATA | |
| | AAAACCCTAAACGACGGGGTTCGGAGAGCTTCCGGGTTGGTGAA | |
| | CACATNGAGGTGCCGGGAGGGTGGCGCGCCCGGAGAGGGCACGG | |
| | AAGCTCCGCGCCCCTTCCCACATACCTTGCCCTATGCATCTCTTCC | |
| | ATCTGGCTGTTCATCTGTATCCTTTGTAATATCCTTTATAATAAACC | |
| | GGTAAACGTAAGTAAAGTGTTTCCCTGAGTTCTGTGAGCCGTT | |
| | CTAGCAAATTATCGAACCCGAGGAGGGGGTCGTGGGAACCCCCGA | |
| | TTTATAGCCGGTCGGTCAGAAGTACGGGTNACAACCTGGGACTTG | |
| | CGACTGGCGTCTGAAGTGGGGGCAGTCTTGTGGGACTGAGCCCTT | |
| | AACCTGTGGGGTCTGACGCTAACTCCAGGTAGNTAGTGTCAGAAT | |
| | TGAATTGAATTGTAGGACACCCAGTTGGTGTCCGCTGGAGAATTG | |
| | NTTGGTGTGTGGGAAAAACCCCACACATTTGGTGTCAGAAGTGTT | |
| | GTGAGAGTAGAGAAAAACAGTTTGTTTTTTCCTACACA | |
| 55 | TGTTGGGGCTCAGAANACGATACCCCAAAGTATGGCGCCTTGGCA | Consensus sequence |
| | TGCTGAGTACTTTGAACTGAAGGAGATTGGAAGGCCTCAGAAGCA | for transposable |
| | GCCTCAGAAGCAAAGTCTCTCTCTGACCTTCTCCCGCCCTCCTGTC | elements that belong |
| | TCCCGCCCCCGTTCTCTCCTCCCCGAAGCGAGTCATAGAAACCA | to MER39 subfamily |
| | GAATTCCTCTTCCCCAAGGCGGGTCATAGAAACTAGAACCCCTCTC | |
| | CCCCAAAGCAAGCCATAAAACCTAGAAANGTCACTCTGACCTTC | |
| | CCCCGCCTTTCTGTNTNGGAGCNGGCCATAAAGAAATTCTCTGACC | |
| | TNCCTTGTCTGANAGTAGGTCATAAGACCCTCATTCCAGAAGGG | |
| | GTCCTGCCCTATACCCGGGAGGAAGGAATGCTACACAGAGAGGCC | |
| | AAGAAGAATCTGAACAGACAGGCCTTGCTGGGTTTCCCCNCTCAG | |
| | TTTATTACCATTAGATCATACCCCTTTTGTCCAATCACATTTCTNCA | |
| | CGACTGTCCATTCTTCATCGAACCTAAGCATAAAAATANAGTT | |
| | TTCCCTGNGTCTTTGGGTCTTCATTTCTGAAGGCTCCCGTGTCACGT | |
| | AAAACTTTGATTAAATAAATTTGTTATGCTTTTCTCTTGTTAA | |
| | TCTGTCTTTTGTTATAGGGGTGTCGGCCGTGANCCTTGCGATGGGT | |
| | AGGAAAGGTATCACACCTTTCTNCCCCTACA | |
| 56 | TGTCAGAGGCGTTTGAACCAGAGCGACTCCATCTTGAATAGGGGC | Consensus sequence |
| | TGGGTAAAATAAGGCTGAGACCTACTGGGCTGCATTCCCAGGAGG | for transposable |
| | TTAGGCATTCTNAGTCACAGGATGAGATAGGAGGTCGGCACAAGA | elements that belong |
| | TACAGGTCACAAAGACCTTGCTGATAAAACAGGNTGCGGTAAAGA | to MER41B |
| | AGCCGGCCAAAACCCACCAAAACCAAGATGGCGACGAAAGTGAC | subfamily |
| | CTCTGGTCGTCCTCACTGCTCATTATACGCTAATTATAATGCATTA | |
| | GCATGCTAAAAGACACTCCCACCAGCGCCATGACAGTTTACAAAT | |
| | GCCATGGCAACGTCAGGAAGTTACCCTATATGGTCTAAAAAGGGG | |
| | AGGAACCCTCAGTTCCGGGAATTGCCCACCCCTTTCCCGGAAAACT | |
| | CATGAATAATCCACCCCTTGTTTAGCATATAATCAAGAAATAAC | |
| | CATAAGNATAGNCAGCCAGCAGCCCACGCCGCTGCTCTGCCTATG | |
| | GAGTAGCCATTCTTTTATTCCTTTACTTTCTTAATAAACTTGCTT | |
| | TCACTTTACTCTATGGACTCGCCCCGAATTCTTTCTTGCGCGAGATC | |
| | CAAGAACCCTCTCTTGGGGTCTGGATCGGGACCCCTTTCCGGT | |
| | AACA | |
| 57 | TGAGACCCTGCTTGNGACACATGTGAAAAATGCAGGGGAAAACCA | Consensus sequence |
| | GTCCCCTGTGGAGTGTGAAAATAATTAAGTGGCAGGCAATTAGAC | for transposable |
| | TGAGGTGGCTCTAGTGCCCTGGGTTCCTACTTAAAAAAAAATCTAA | elements that belong |
| | CTCAAATGCATTTTTTGTAAATTACTACNTTAGGGGAAAACAAA | to MER57E3 |
| | ATTCAGGCTTAACCAACCATAAACCGCCAATTAANCTCTGATTACA | subfamily |
| | TAACCAGGAAATTTCCACCTGGATNGTACAAATNAAGAAACTAC | |
| | GTAACTGTACCTAACCAATTATTGAATTTGGTTTGCTTCNTCATGC | |
| | ACCTTATAAAAGCCTTTCCTTCAAGCCCCTCCCGTGGACCACAA | |
| | ACTACAAACCATAGCTGGGTGCTCTACGATTCNTGAATCACTCTTT | |
| | GATTAAATTCTTTAATATTTTTGCGGTGACTCCCATAAATTTTT | |
| | AACAGGAGAAAGGAGGGACTGGGGACCCCACGGACCA | |
| 58 | TGAGGTAGGAGACCGGCAGGACTTGTTTTCTGGTCACAACCCTGCT | Consensus sequence |
| | GACCAAAACAGGATCTGGTCCAGACAGGATAAAGTGAAGAAACC | for transposable |
| | GGCAGGAACCAGCAGATGGCGACGAAAGCGATCCCTAGCTGCCCT | elements that belong |
| | CATTGCTCATTAGCATAAGACACTCCCACCAGCGCCATGACAGTT | to MER66C |
| | TACAAATGCCATGGCAACGACCCGGAAGTTACCACCCCTTTCCATG | subfamily |
| | GCAACGACCCGGAAGTTACCGCCCCTTTCCTAGAAAGTTCTAAA | |
| | TAACCCGCCCCTCAATTTGCATTGACCCGCCCCTTAATTTGCATGT | |
| | AATTGAAAGTGGGTNTAAGTGAGTATAAATACAGTTGCCAAGAG | |
| | CCCATACGTTGCCGACTCTGGGCGCACTGCCTATGAGTTAGCCCTG | |
| | CTCCGCAAGGAGCAGTACCGTTCAATAAAAGATTGCTGTCTAAC | |
| | ACCACCGGCTCGCCCTTGAATTCTTTCCTGGGCGAAGCCAAGAACC | |
| | CTCCCGGGCTAAGCCCCAATTTTGGGGCTCGCCTGTCCTGCATC | |
| | ATCTGGCTACCATCA | |
| 59 | TGAGGTAGGAGATCAGCAGGACTTGTTTTCCGAGCACCGGTCACG | Consensus sequence |
| | ACCCTGCTGATCAAAACAGGATGTAGCAAAGAAACCGGCCAAAAC | for transposable |
| | CAGCTAGGACCAGGAATTATAATGCATTNGCATAAGACACTCCCA | elements that belong |
| | CCAGCGCCATGACAGTTTACAAATGCCATGGCAACGACCCGGAAG | to MER66D |
| | TTACCTTATATGGTTCCGGGAACTCCCCGCCCCTTTTCCAGAAAGT | subfamily |
| | TCGTGAATAACCCGCCCCTTATTTAGCATATAATTAAGAGTAGG | |
| | TATAAATATAGCTAGCCAGCAATCCACGAGTGCTACTCTGGGCCG | |
| | CTCTGCCTATGGGGTAGCCCTGCTCTGTCTATGGAGCAGCCATTT | |
| | TGCTGTACACTGTTGCTCTAATAAACTTGCTTTCTTTCACTGTCGGC | |
| | TCGCTCTTGAATTCTTTCCTGNGCGAAGCCAAGAACCCTCCCG | |
| | GGCTGAGCCCCAATTTTGGGGTTCGCCTGCATCA | |
| 60 | TGTGCAGAAAAGAGTTAACATAGCAGGCCTGAGACTGCTATCCTT | Consensus sequence |
| | AGAAAGGCCTGCTTGCAAGGTTGGCCCTTGGCTGGCGTCTGGGAA | for transposable |
| | CTTGGATTTCGGGAGGGTTCCCACCATTCCCGNAACTGATAAGAGT | elements that belong |
| | GGCTCACTGTGCCTAAACTGTTTGTACAAACAATGTGGTTTATG | to MER68 subfamily |
| | CTGAACACCTGCTTTCCTTCTGGGAGTCTGGAATTTTGGTACGTGC | |
| | TAGGCAGAGGGTGCCTACGTGACCAGCCCCCAATAAAAACCCTG | |
| | GGCACTGAGTCTCTAATGAGCTTCCCTGGTAGACAACATTTCACAC | |
| | GTGTTGTCACAACTCGTTGCTGGAGGAATTAAGCGCGTCCTGTG | |
| | TGACTCCACTGGGAGAGGACTCTTGGAAGCTTGCGCCTGGTTTCCT | |
| | CCGGACTTCGCCCCATGCGCCTTTTCCCTTTGCTGATTTTGCTT | |
| | TGTATCCTTTCGCTGTAATAAATCATAGCCGTGAGTACGACTATAT | |
| | GCTGAGTCCTGTGAGTCCTCCTAGCGAATCACCGAACCTGGGGG | |
| | TGGTCTTGGGGACCCCCGACACA | |
| 61 | TGTGTAGTAAAGAATTTAACCTTGCCCAAAGAGAGGTCTGGCCTTT | Consensus sequence |
| | GCCCTCGGCTCCTGGGAGGTAATCTCTAAGCCCTTGGAATGTCN | for transposable |
| | TGCCTGATAGGAGTGTCTTTGTTTGCCTGGGGGCCTTGGGCCACGC | elements that belong |
| | CGGATAGTCTAACAATGTGATTTAGGGTGGGGGCTGGCCACGCC | to MER77B |
| | AGAAAGACCAACNATGTGATTTAGGGTGGGGGCTTTGGGTCACGC | subfamily |
| | GGTATCAGCCCGACTCCGGAGGGNCTGGAGACTGAGNTCAGCCAC | |
| | GTGGGCAATCAATCAATCATGCCTACGTGATGGAGCCCCAATAAA | |
| | AACTCTGGACACCGAGGCTCGGGTGAGCTTCCCTGGTTGGCAATA | |
| | CTCCGTGCGTATTGTCACACATCGATGCCGGGAGAGTAACGCTGTC | |
| | CCTGACTCCACGGGGAGAGGACAACTGGAAGCTCCGCGTTTGGA | |
| | ACCCTCCCGGACTCTGCCCTATGTACCGCGCGCCTCTTCCCTTGGC | |
| | TGATTTTAATCTGTATCCTTTCCCTGTAATAAACCGTAACCGTG | |
| | AGTATAACAGCTTTCAGTGAGTTCTNCGTGAGTCCTTCTAGCGAAT | |
| | TATCGAACCTGAGGGTGGTTTTGGGAACCCCCCGAACTTGCAGT | |
| | TGGTGTCAGAAGTGAGGGCGGTCTTGTGTGGACTGTGTCCCTCTAA | |
| | CTTTGCAGTTGGCTAACTTTCACA | |
| 62 | GAAATTGGTACCGAGAGTGGGGGTGCTGCTATAACAAATACCTAA | Consensus sequence |
| | AAATGTGGAAGCGGCTTTGGAACTGGGTAATGGGTAGAGGCTGGA | for transposable |
| | AGAGTTTNGAGGTGCATGCTAGAAAAAGCCTANATTGCCGTGAAC | elements that belong |
| | GGANCGTTAAGGGCGATTCTGGTGAGGGCTCAGAAGAAGAGGAG | to MLT-int |
| | AGCTGTAGAGAAAGCCTCAATCTTCTTAGAGATTACCTAAGTGGTC | subfamily |
| | GTGANCAGAATGTTGGTAGAAATATGGACGGTAAAGGCCATTCTG | |
| | ATGAGGTCTCAGACGGAAATGAGGAACATGTTATTGGAAACTGGA | |
| | GGAAAGGCCATCCTTGTTATAAAGTGGCAAAGAACTTGGCTGAAT | |
| | TGTGTCCGTGTCCTAGNGCTTTGTGGAAGGCAGAACTTANGAGCG | |
| | ATGAANTAGGATATTTGGCGGAAGAAATNTCTAAGCAAAGTGTTG | |
| | AGGGTGCNGCGTGGCTTCTCTTGACTGCTTATAGTAAAATGCGAGA | |
| | AGAGAGAAATGANTTAAAGACGGAATTTATAATCAAAAGGGAAG | |
| | CAGAACNTAAAGATTTGGAAAATTCTCAGCCTGGCCATGTTGNAA | |
| | AGAATGAAAAAGCGTGTTCGGGAGAGAANACCAAGGGTGTGGCC | |
| | AAGCGACCGTTTGATAAGGAGATTAGTATGGATAGAAGGAAGCCA | |
| | GGTGCTATTCATCAAGACAATGGAAGAATGACCCCGAAGGCATTT | |
| | CGGAGATCTTCGAGGCTGCCCCTCCCATCACAGGCCCAGAGTGCN | |
| | AGGGCCTNGAGGGCAGAACGGTTTCAAGGGCAGGNNCCANTCCCC | |
| | ACTGCCCAGNGCCGCCTCAGTCTGCTCCCCGTCTTCGGCCGCCCGN | |
| | NCAGCTGTGGCTCAAGCGGGCCCAGGTGCGGCTCGGGCCGCCGCT | |
| | CCGGAAGGCACAAGCCGTAAACCTTGGCGGCGTCCACGTGGTGCT | |
| | AACTCTGCAGGCGCGCAGAGTGCANGAGCTGTGGGGGCATGGCTN | |
| | CCTCCACCTAGATTTCAAAGATGCGAGACCTGGGGCCCAGGCAGA | |
| | GANCTCGCGGGGCAGGGCCACCGCAGAGAGCCCCCACTAGGGCA | |
| | ATGCCCAGTGGAGCCGTGGGGTCGGGNCTGCAGAGAGCCCCCACT | |
| | AGGGCAATGCCTAGTGGAGCCGTGGGGGCGGGGCCGCCCCCGAGA | |
| | CCCCAGAACTGTAGAGCCACCAGCGTGCAACNCCAGCCTGGGAGA | |
| | GCCGCAGGCACGNGACTCCAACCCGTGAGAGCTGCCGCGTGGGCT | |
| | GCGCCCAGCAAAGCCATGGGGGCGGGGCTGCCCGGGGCCTTGGGG | |
| | GCCCAACCCCCGCCCCAGTGTGTCCGGAAGGCGGGACATGGAGTC | |
| | AAAGAAGATTATTCTCGAGCCTTAAGATTTAATGTTGTTTGCCCTG | |
| | TTGGGTTTTGGACTTACTTGGGACCTGTTACCCCTTTCTTCTTTC | |
| | CTATTTCTCCCTTTTGGAATGGGAATGTCTATCCTATGCCTGTCCCA | |
| | CCATTGTATTTTGGAAGCACATAACTTGTTTGATTTCACAGGC | |
| | TCACAGCTGGAGAGGAATTTTGCCTCAGGATGAATCGTACCTTGA | |
| | GTCTCACCCATATCTGATTTAGATGATATTTAGATGAGACTTTGG | |
| | ACTTTAGACTTTNGAGTTGATGCTGGAACGAGTTAAGACTTTNGGG | |
| | GCTATTGGGATGGAATGAATGTATTTTGCATGTGAGAAGGACAT | |
| | GAATTTTGGGGGGCCAGGGGCGGAA | |
| 63 | TGCTATGGACTGAATGTTTGTGTCCCCCCAAAATTCATATGTTGAA | Consensus sequence |
| | GCCCTAACCCCCAATGTGATGGTATTTGGAGGTGGGGCCTTTGG | for transposable |
| | GAGGTAATTAGGNTTAGATGAGGTCATGAGGGTGGGGCCCTCATG | elements that belong |
| | ATGGGATTAGTGCCCTTATAAAAGAGACACCAGAGAGCTCTCTTN | to MLT1A |
| | CCCCTTCCGCCATGTGAGGACACAGCGAGAAGGCGCCGTCTGCAA | subfamily |
| | GCCAGGAAGAGAGCCCTCACCAGAAACCGAATCTGCCGGCACCTT | |
| | GATCTTGGACTTCCCAGCCTCCAGAACTGTGAGAAATAAATTTCTG | |
| | TTGTTTAAGCCACCCAGTCTATGGTATTTTGTTATGGCAGCCCG | |
| | AGCNGACTAAGACA | |
| 64 | TGCTATGGTCTGAATGTTTGTGTCCCCCCAAAATTCATATGTTGAA | Consensus sequence |
| | ACCTAACCCCCAANGTGATGGTATTAGGAGGTGGGGCCTTTGGG | for transposable |
| | AGGTGATTAGGTCATGAGGGCGGAGCCCTCATGAATGGGATTAGT | elements that belong |
| | GCCCTTATAAAAGAGGCCCCAGAGAGCTCCCTCGCCCCTTCCGCC | to MLT1A0 |
| | ATGTGAGGACACAGCGAGAAGGCGCCGTCTATGAACCAGGAAGC | subfamily |
| | GGGCCCTCACCAGACACCGAATCTGCCGGCGCCTTGATCTTGGACT | |
| | TCCCAGCCTCCAGAACTGTGAGAAATAAATTTCTGTTGTTTATAAG | |
| | CCACCCAGTCTATGGTATTTTGTTATAGCAGCCCGAACGGACTA | |
| | AGACA | |
| 65 | TGCTATGGTTTGAATGTTTGTGTCCCCTCCAAAATTCATGTGTTGA | Consensus sequence |
| | AACTTAATCCCCAATGCGATAGTATTAAGAGGTGGGGCCTTTAG | for transposable |
| | GAGGTGATTAGGTCATGAGGGCTCCGCCCTCATGAATGGGATTAG | elements that belong |
| | TGCCCTTATAAAAGGGGCTTGAGGGAGCGAGTTCGGCCCTTCTTG | to MLT1A1 |
| | CCCTTCCGCTCTTCCGCCATGTGAGGACACAGCGTTCCTCCCCTCC | subfamily |
| | GGAGGATGCAGCAACAAGGCGCCATCTTGGAAGCAGAGAGCAGG | |
| | CCCTCACCAGACACCGAACCTGCCGGCGCCTTGATCTTGGACTTCC | |
| | CAGCCTCCAGAACTGTGAGAAATAAATTTCTGTTCTTTATAAAT | |
| | TACCCAGTCTCAGGTATTTTGTTATAGCAGCACAAACGGACTAAGA | |
| | CA | |
| 66 | TGTTATGGGCTGAATTGTGTCCCCCCAAAATTCATATGTTGAAGTC | Consensus sequence |
| | CTAACCCCCAGTACCTCAGAATGTGACTGTATTTGGAGATAGGG | for transposable |
| | CCTTTAAAGAGGTAATTAAGTTAAAATGAGGTCATTAGGGTGGGC | elements that belong |
| | CCTAATCCAATATGACTGGTGTCCTTATAAGAAGAGGAGATTAGG | to MLT1B |
| | ACACAGACACGCACAGAGGGAAGACCATGTGAGGACACAGGGAG | subfamily |
| | AAGGCGGCCATCTGCAAGCCAAGGAGAGAGGCCTCAGAAGAAAC | |
| | CAACCCTGCCGACACCTTGATCTCGGACTTCCAGCCTCCAGAACTG | |
| | TGAGAAAATAAATTTCTGTTGTTTAAGCCACCCAGTCTGTGGTACT | |
| | TTGTTATGGCAGCCCTAGCAAACTAATACA | |
| 67 | TGTTATGGGTTGAATTGTGTCCCCCCAAAATTCATATGTTGAAGTC | Consensus sequence |
| | CTAACCCCCAGTACCTCAGAATGTGACCTTATTTGGAAATAGGG | for transposable |
| | TCNTTGCAGATGTAATTAGTTAAGATGAGGTCATACTGGAGTAGG | elements that belong |
| | GTGGGCCCCTAATCCAATATGACTGGTGTCCTTATAAAAAGGGGA | to MLT1C |
| | AATTTGGACACAGACACGCACACAGGGAGAACGCCATGTGAAGAT | subfamily |
| | GAAGGCAGAGATCGGGGTGATGCNTCTACAAGCCAAGGAACGCC | |
| | AAAGATTGCCAGCAAACCACCAGAAGCTAGGAGAGAGGCATGGA | |
| | ACAGATTCTCCCTCACAGCCCTCAGAAGGAACCAACCCTGCCGAC | |
| | ACCTTGATCTCGGACTTCTAGCCTCCAGAACTGTGAGACAATAAAT | |
| | TTCTGTTGTTTAAGCCACCCAGTTTGTGGTACTTTGTTACGGCAGC | |
| | CCTAGNAAACTAATACA | |
| 68 | TGTTGTGGGTTGAATTGTGTCCCCCAAAAAGATATGTTCAAGTCCT | Consensus sequence |
| | AACCCCCGGNACCTGTGAATGTGACCTTATTTGGAAATAGGGTC | for transposable |
| | TTTGCAGATGTAATCAAGTTAAGATGAGGTCATACTGGATTAGGGT | elements that belong |
| | GGGCCCTAATCCAATGACTGGTGTCCTTATAAGAAGAGGGAAAT | to MLT1C2 |
| | NTGGACACAGAGACACACAGGGAGAAGGCCATGTGAAGACGGAG | subfamily |
| | GCAGAGATTGGAGTGATGCGGCTACAAGCCAAGGAACGCCAAGG | |
| | ATTGCCGGCAACCACCAGAAGCTAGGAAGAGGCAAGGAAGGATT | |
| | CTCCCCTAGAGCCTTCAGAGGGAGCACGGCCCTGCCGACACCTTG | |
| | ATTTCGGACTTCTGGCCTCCAGAACTGTGAGAGAATAAATTTCTGT | |
| | TGTTTTAAGCCACCCAGTTTGTGGTANTTTGTTACGGCAGCCCTAG | |
| | GAAACTAATACA | |
| 69 | TGTGGTAGGCAGAATAATGGCCCCCCAAAGATGTCCACGTCCTAA | Consensus sequence |
| | TCCCCGGAACCTGTGAATATGTTACCTTACATGGCAAAAGGGACT | for transposable |
| | TTGCAGATGTGATTAAGTTAAGGATCTTGAGATGGGGAGATTATCC | elements that belong |
| | TGGATTATCCGGGTGGGCCCAATGTAATCACAAGGGTCCTTATA | to MLT1D |
| | AGAGGGAGGCAGGAGGGTCAGAGTCAGAGAAGGAGATGTGACGA | subfamily |
| | CGGAAGCAGAGGTCGGAGTGACGACGTTGCTGGCTTTGAAGATGG | |
| | AGGAAGGGGCCACGAGCCAAGGAATGCGGGCGGCCTCTAGAAGC | |
| | TGGAAAAGGCAAGGAAACGGATTCTCCCCTAGAGCCTCCAGAAGG | |
| | AACGCAGCCCTGCCGACACCTTGATTTTAGCCCAGTGAGACCCATT | |
| | TCGGACTTCTGACCTCCAGAACTGTAAGATAATAAATTTGTGTTGT | |
| | TTTAAGCCACTAAGTTTGTGGTAATTTGTTACAGCAGCAATAGGAA | |
| | ACTAATACA | |
| 70 | TGTGGTAGGCAGAATTCTAAGATGGCCCCCAAGATTCCCGCCCCCT | Consensus sequence |
| | GGTGTACACGCCCTGTATAATCCCCTCCCCTTGAGTGTGGGCGG | for transposable |
| | GACCTGTGAATATGATGGGATNTCACTCCCGTGATTAGGTTACGTT | elements that belong |
| | ATATGGCAAAGGTGAAGGGATTTTGCAGATGTAATTAAGGTCCC | to MLT1E |
| | TAATCAGTTGACTTTGAGTTAATCAAAAGGGAGATTATCCTGGGTG | subfamily |
| | GGCCTGACCTAATCAGGTGAGCCCTTTAAAAGAGGGTCTAGAGG | |
| | TCAGAGACGGAAGAAGTCAGAGAGATTCGAAGCAGCAGAGACGC | |
| | TCTCCTGCTGGCCTTGAAGAAGCAAACTGCCATGTTGTGGAGAGG | |
| | GCCACGTGGCAGGGAACGGCGGGCGGCCTCTAGGAGCTGAGGGCC | |
| | TCAGTCCTACAACCGCAAGGAACTGAATTCTGCCAACAACCAGTG | |
| | AGCTTGGAAGAGGACCCCGAGCCTCAGATGAGACCGCAGCCCCGG | |
| | CCGACACCTTGATTTCAGCCTNGTGAGACCCTGAGCAGAGGACCC | |
| | AGCTAAGCCGTGCCCGGACTCCTGACCCACGGAAACTGTGAGATA | |
| | ATAAATGTGTGTTGTTTTAAGCCGCTAAGTTTGTGGTAATTTGTTA | |
| | CGCAGCAATAGAAAACTAATACA | |
| 71 | TGTGGTAGGCAGAATTCTAAGATGGCCCCCAAGATTCCCACCCCCT | Consensus sequence |
| | GGTGTACATGCCCTGTATAATCCCCTCCCCTTGAGTGTGGGCGG | for transposable |
| | GACCTGTGAATATGATGGGATATCACTCCTGTGATTAGGTTACGTT | elements that belong |
| | ATATGGCAAAGGTGAAGGGATTTTGCAGATGTAATTAAGGTCCC | to MLT1E1 |
| | TAATCAGTTGACTTTGAGTTAATCAAAAGGGAGATTATCCTGGGTG | subfamily |
| | GGCCTGACCTAATCAGGTGAGCCCTTTAAAAGAGGGTCTGGAGG | |
| | TCTTTCTGAAGAAGTCAGAGAGATTCGAAGCAGCAGAGATGCTCT | |
| | CCTGCTGGCCTTGAAGAAGCAAGCTGCCATGTTGTGGAGAGGGCC | |
| | ACGTGGCAGGGAACNGCGGGCGGCCTCTAGGAGCTGAGGGCCTCA | |
| | GTCCTACAGCCGCAAGGAANTGAATTCTGCCAACAACCTGAGTGA | |
| | GCTTGGAAGNGGACCCTGAGCCTCCAGATGAGAACGCAGCCCCGG | |
| | CCGACACCTTGATTNCAGCCTTGTGAGACCCTGAGCAGAGGACCC | |
| | AGCTAAGCCGTGCCCGGACTCCTGACCCACGGAAACTGTGAGATA | |
| | ATAAATGTGTGTTGTTTTAAGCCGCTAAGTTTGTGGTAATTTGTT | |
| | ACGCAGCAATAGAAAACTAATACA | |
| 72 | TGTGGTAGGCAGAATTCTAAGATGGCCCCCAAGATTCCCGCCCCCT | Consensus sequence |
| | GGTGTACACGCCCTGTATAATCCCCTCCCCTTGAGTGTGGGCGG | for transposable |
| | GACCTGTGAATATGATGGGATATCACTCCCGTGATTAGGTTACATT | elements that belong |
| | ATATGGCAAAGGTGAAGGGATTTTGCAGATGTAATTAAGGTCCC | to MLT1E1A |
| | TAATCAGTTGACTTTGAGTTAATCAAAAGGGAGATTATCCTGGGTG | subfamily |
| | GGCCTGACCTAATCAGGTGAGCCCTTAAAAGAGGCATGGGCCCT | |
| | CCAGAGAGAAGAACAGAGAGATTCTCCTGCTGGCCTTGAAGAAGC | |
| | AAGCTGCCATGTTGTGAGAGGGCCTATGGAGAGGGCCACGTGGCA | |
| | AGGANCTGNGGGCGGCCTCTAGGAGCTGAGAGCGGCCCCCGGCCG | |
| | ACAGCCAGCAAGAAAACGGGGACCTCAGTCCTACAGCCGCAAGG | |
| | AANTGAATTCTGCCAACAACCTGAATGAGCTTGGAAGNGGACCCT | |
| | AAGCCTCAGATGAGAACGCAGCCCTAGCCGACACCTTGATTNCAG | |
| | CCTTGTGAGACCCTGAGCAGAGGACCCAGCTAAGCCGTGCCCGGA | |
| | CTCCTGACCCACAGAAACTGTGAGATAATAAATGTGTGTTGTTTTA | |
| | AGCCGCTAAGTTTGTGGTAATTTGTTACGCAGCAATAGAAAACTA | |
| | ATACA | |
| 73 | TGTGGTAGGCAGAATGGCCCCCCAAAGATGTCCACGCCCTAATCC | Consensus sequence |
| | CCGGAACCTGTGAATATGTTACGTTACATGGCAAAAGGGACTTTG | for transposable |
| | CAGATGTAATTAAGGTTACGGACCTTAAAATAGGGAGATTATCCT | elements that belong |
| | GGATTATCCGGGTGGGCCCAATCTAATCACATGAGCCCTTAAAAG | to MLT1E2 |
| | CAGAGAACTTTCTCCGGCTGGAGTCAGAGAGATGCGGCAGAAGGG | subfamily |
| | GAAGTCAGAGAGATTCGAAGCGTGAGAAGGACTCGACGCGCCGTT | |
| | GCTGGCTTTGAAGATGGAGGGGGCCACGTGNCAAGGAATGCGGGC | |
| | GGCCTCTAGGAGCTGAGAGCGGCCCCCGGCTGACAGCCAGCAAGG | |
| | AAACGGGGACCTCAGTCCTACAACCGCAAGGAACTGAATTCTGCC | |
| | AACAACCTGAATGAGCTTGGAAGCGGATTCTTCCCCAGAGCCTCC | |
| | AGANAAGAGCNCAGCCCNGCCGACACCTTGATTTCGGCCTTGTGA | |
| | GACCCTGAGCAGAGAANCCAGCCGAGCCCACCCGGACTTCTGACC | |
| | TACAGAACTGTGAGATAATAAATNTGTGTTGTTTTAAGCCGCTAAG | |
| | TTTGTGGTAATTTGTTACGGCAGCAATAGAAAACTAATACA | |
| 74 | TGTGGTAGGCAGAATTCTAAGATGGCCCCCAAGATTCCCGGCCCCT | Consensus sequence |
| | GGTGTACACACACCTTCTCCCAGTTATTCAATCAAACACTAATC | for transposable |
| | TAGGTGCTGCTGTGAAGGGATTTTGCAGATGTAATTAAGGTCCCAA | elements that belong |
| | ATCAGTTGACCTTAAGATAGGGAGATTATCCTGGGTGGGCCTGA | to MLT1E3 |
| | CCTAATCACGTGAGCCCTTAAAAGGGACTGGGCTCTTCCTGGAGA | subfamily |
| | AAGAGATTCGAAGCGTGAGAGGGATTCGACGCGAGGGAGATTCTC | |
| | CGTTGCTGGCTTTGAAGATGGAGGGGGCCACGTGGCAAGGAATGC | |
| | GGGCGGCCTCTAGGAGCTGAGAGCGGCCCCCGGCTGACAGCCAGC | |
| | AAGGAAACGGGGACCTCAGTCCTACAACCGCAAGGAACTGAATTC | |
| | TGCCAACAACCTGAATGAGCTTGGAAGCGGACCCTGAGCTCCAGA | |
| | TGAGAACNCAGCCCGGCCGACACCTTGATTTCAGCCTTGTGAGAC | |
| | CCTGAGCAGAGAACCCAGCCACGCCGTGCCCGGACTTCTGACCTA | |
| | CAGAACTGTGAGCTAATAAATGGGTGTTGTTTTAAGCCGCTAAGTT | |
| | TGTGGTAATTTGTTACGCAGCAATAGAAAACTAATACA | |
| 75 | TGTGGTAGCCAGCCTCCAAGATGGCCCCCAATGATCCCCGCCTCCT | Consensus sequence |
| | GGTATTCACGCCCTTGTGTAGTCCCCTCCCACACTGAATAGGGC | for transposable |
| | TGGCCTGTGTGACCAATAGGATATNGCGGAAGTGACGGTGTGTGA | elements that belong |
| | CTTCCGAGGCTAGGTCATAAAAGACATTGCGGCTTCCGCCTTGCT | to MLT1F subfamily |
| | CTCTCTTGGATCACTCGCTCTGGGGGAAGCCAGCTGCCATGTCGTG | |
| | AGGACACTCAAGCAGCCCTGTGGAGAGGCCCACGTGGCGAGGAA | |
| | CTGAGGCCTCCTGCCAACAGCCAGCACCAACTTGCCAGCCATGTG | |
| | AGTGAGCCATCTTGGAAGCGGATCCTCCAGCCCCAGTCAAGCCTT | |
| | CAGATGACTGCAGCCCCGGCCGACATCTTGACTGCAACCTCATGA | |
| | GAGACCCTGAGCCAGAACCACCCAGCTAAGCCGCTCCCGAATTCC | |
| | TGACCCACAGAAACTGTGTGAGATAATAAATGTTTATTGTTGTTTT | |
| | AAGCCGCTAAGTTTTGGGGTAATTTGTTACGCAGCAATAGATAA | |
| | CTAATACA | |
| 76 | GATTTTGGTACCTGGAAGTGGGGTGCTGCCGTAACAAATACCTAA | Consensus sequence |
| | AAATGTGGGAGTGGCTTTGGAACCGGGCAGTGGGCGGAAGCTGGA | for transposable |
| | AGGATTTTGAGGAGCGTGNTAGNGAAAGCCTAAANTGCCTTGAAC | elements that belong |
| | AGACTGTTAGTAGAAATCTGGACTTTGAGGANGCTGCCGGTGAGG | to MLT1F-int |
| | GCTCAAAGGGAAGTGAGGAANATGTTATTGGAAACTGGAGGAAA | subfamily |
| | GGGGATCCTTGTTATGTAGTGGCAGAAAGCTTAGCAANACTGTCG | |
| | CCTGCAGTTATGTGGAAAGTAGAAAATGTANCTAATGAACTNGGT | |
| | GATCTAGCTAAGGAGATTTCCAAGCAAAGTGTTGAAGGTGCCGCC | |
| | TGGTTTCTTCTTGCTGCTTATAGTAAAATGCGAGAGGAGAGAGATA | |
| | AACTGAGGGAAGAACTGTTAAACAAAAAGGAGCCAGGACTTGAT | |
| | GGTTTTGAAAATTCTCAGCCTCTCCAGATGGCAAANGATGCTAAA | |
| | ATTAAGAAATGGCTTCCGAGCAAAGATCAAATCCAGGGCACTGTC | |
| | AGGAAAACATGGTCTAAAGATGAAGCCGAGGGTGTGACTGTAAAA | |
| | TCCTTTGTTAAGACCTCAGAAAGATCAAAGGTGGTGCCTCAGAGT | |
| | ACTATTCAGTCANACAAAAGGCCCTTTAAAGAGATTAAGGGTGTG | |
| | CCTCACAGATCCTCTCAATCAAACAATAGGGCTTCTAAGAAGCTTA | |
| | AGGGCGTTGTCCCTCAGCCGTCTCAGCAGAAGCCCAAGGTAGAGA | |
| | AGGGCTTATCTCGAAGAGATTTGTGGGTGTGGCTTTTGTCTAATG | |
| | GAGTGAACCCCAGTAAGATTCACAGGAGACCCACAAAGTTTTTAA | |
| | GAGAATTATATCAGCAGAAACACTGCCAGCTTGGACTGAAAGGGA | |
| | CAGAGACAGTACAAAATGAAAAGAGGCCTTTGGACCCCCAAANTT | |
| | CTACNGGCAGGAAGCAGGCTGANAAAACTACTCAGCTGCAAACAC | |
| | GTGCTACCTTTCATGAAAAAGGAAGGATGACTCAGAGGGCGGAAC | |
| | CAAGAGCCCAGAGGGCGGAGCCAAGAGCCACGGAGAATTATTCCC | |
| | AGGCCTTGAGACCTAATCAAGGAACTTCCAACATTTGCCCGGCTG | |
| | GATTTCAGAATTGCTATGGACCAGTGACTCCTTTGTGCCTCCCAT | |
| | TTTCCCCCTTTTTGAACGGGAATGTCTATAGCGGTTATCCTATGCCT | |
| | GTCCCACCATTGTATGTTGGGTGTGTNGGGGGCAGATAACTTG | |
| | TCTCTTTAGTTTCACAGGTCTNCAGATCGAGAGGAACTGTACTCGA | |
| | GGAGCTGTACTTAAGGAACTACACCCGAGGAGCCTCATCCACAC | |
| | CTGGACCTGATTTAGATGATGAGATTCTGGACTTTGAGCTGATGCT | |
| | GTAATGGGATGAGACTTTTGGGGATCTTGGGAGGGGGGTGAGTG | |
| | TATTTTGCATGTGGGAGGGACGTGAATCATTGGGGGCCAGAGGGC | |
| | AGAC | |
| 77 | TGTGGTGGTTTTAAAATATGTCCACAAATTCTTTGATACTCCTCCCT | Consensus sequence |
| | TCAAGAGGTGGAGCCTAATTCCCCTCCCCTTGAGTGTGGGCTG | for transposable |
| | GACTTAGTGACTCGCTTCTAACGAATAGAATATGGCGGAAGTGAC | elements that belong |
| | GGTGTGTGACTTCCGAGACTAGGTCATAAAAGGCATTGCGGCTTC | to MLT1F1 |
| | CTCCTTGCTCTCTCTCTTGGATCACTCGCTCTGGGGGAAGCCAGCT | subfamily |
| | GCCATGTCGTGAGGACACTCAAGCAGCCCTATGGAGAGGCCCAC | |
| | GTGGCGAGGAACTGAGGCCTCCTGCCAACAGCCAGCAAGGAACTG | |
| | AGGCCTCCTGCCAACAGCCATGTGAGTGAGCCATCTTGGAAGCGG | |
| | ATCCTCCAGCCCCAGTCAAGCCTTCAGATGACTGCAGCCCCGGCC | |
| | GACATCTTGACTGCAACCTCATGAGAGACCCTGAGCCAGAACCAC | |
| | CCAGCTAAGCCGCTCCCGAATTCCTGACCCACAGAAACTGTGTGA | |
| | GATAATAAATGTTTGTTGTTTTAAGCCGCTAAGTTTTGGGGTAAT | |
| | TTGTTACGCAGCAATAGATAACTAATACA | |
| 78 | TGTGGTAGGCAGCCTCTAAGATGGCCCCCAATGATCCCCGCCTCCT | Consensus sequence |
| | GGTATTCACGCCCTTGTGTAATCCCCTCCCCTTGAGTGTGGGCT | for transposable |
| | GGACCTAGTGACTCGCTTCTAACGAATAGAATACGGCAAAAGTGA | elements that belong |
| | TGGGATGTCACTTCCGAGATTAGGTTACAAAAAGACTGTGGCTTC | to MLT1F2 |
| | CGTCTTGCGCGCCCTCTCTTGCTCTCTCGCTTGCTCGCTCTGANGGA | subfamily |
| | AGCNAGCTGCCATGTTGTGAGCTGCCCTATGGAGAGGCCCACG | |
| | TGGCAAGGAACTGAGGGCGGCCTCCGGCCAACAGCCAGCGAGGA | |
| | ACTGAGNCCTGCCAACAACCACGTGAGTGAGCTTGGAAGCGGATC | |
| | CTCCCCCAGTCGAGCCTTNAGATGACCGCAGCCCCGGCCGACACC | |
| | TTGATTGCAGCCTCGTGAGAGACCCTGAGCCAGAGGCACCCAGCT | |
| | AAGCCGCGCCCGGATTCCTGACCCACAGAAACTGTGAGATAATAA | |
| | ATGTNTGTTGTTTTAAGCCGCTAAGTTTTGGGGTAATTTGTTACGC | |
| | AGCAATAGATAACTAATACA | |
| 79 | TGTGGCGGTTTTTAAAACATGGCCGCAAATTCTTTGACACTCCTCC | Consensus sequence |
| | CATCGAGAGGTGGGGTCTATGTCCCCTCCCCTTGAATCTGGGCG | for transposable |
| | GGCTTGTGACTGCTTCGACCAATAGAGTACGGCGGAAGTGACGCT | elements that belong |
| | GTGTGACTTCCGAGGCTAGGTCATAAAAGGCCATGCAGCTTCCGC | to MLT1G |
| | CTTGTTCGCTGGAACACTCGCTCTTGGAGCCCTGAGCCGCCATGTA | subfamily |
| | AGAAGTCCGACTACCCTGAGGCCGCCATGCTGTGAGGAAGCCCA | |
| | AGCCACATGGAGAGGCCACGTGTAGGCGCTCCGGTCGACAGTCCC | |
| | AGCTGAGCCCAGCCTTCGAGTCATCCCAGCCCAGGCGCCAGACAT | |
| | GTGAGTGAAGAAGCCTCCAGATGATTCCAGCCCCCAGCCGTTCGA | |
| | GTCACCCCCAGCCGTTCGAGTCTTCCCAGCTGAGGCCCCAGACAT | |
| | CGTGGAGCAGAGACAAGCCATCCCCGCTGTGCCCTGTCCGAATTC | |
| | CTGACCCACAGAATCCGTGAGCATAATAAAATGGTTGTTGTTTTA | |
| | NGCCACTAAGTTTTGGGGTGGTTTGTTACGCAGCAATAGATAACTG | |
| | GAACA | |
| 80 | TGTGGCAGATTGTATTTTCCAAAGATGGCCGCAACAATATCTCCCA | Consensus sequence |
| | TCCCACATGCTCTTCTTACAATGTGACNTTGACACTCCTCCCAT | for transposable |
| | CGAGAGGTGGGGTCTATGTTCCCTCCCCTTGAATCTGGGCGGGCCT | elements that belong |
| | TTGTGACTGCCTCGACCAATAGAATGCGGCGGAAGTGACGCTGT | to MLT1G1 |
| | GTGACTTCCGAGGCTAGGTCATAAAAGTGCCATGCAGCTTCCGCCT | subfamily |
| | TGCTCTCTTGGGACGCTCGCTCTTGGAACCCAGCCGCCATGCTG | |
| | TGAGGAAGCCCAAGCAGCCCATGGAGAGGCCCACGTGGAGAGGA | |
| | ACTGAGGCTTCCGGCCGACAGCCCCAGCTGAGNTCCCAGCCGACA | |
| | GCCAGCATCAACTTGCCAGNCATGTGAGTGAGCCATCTTGNAGGT | |
| | GATTCCAGCCCCCAGCCTTTGAGCCGCCCCAGCTGACGCCGCGTG | |
| | GAGCAGAGACGAGCCGTCCCCGCCGAGCCCTGCCCAAATTGCAGA | |
| | TTCGTGAGCAAAATAAATGATTGTTGTTGTTTTAAGCCACTAAGTT | |
| | TTGGGGTGGTTTGTTACGCAGCAATAGATAACCGGAACA | |
| 81 | TGTGGCAGATTGTATTTTCCAAAGATGGCCGCAACAATATCTCCCA | Consensus sequence |
| | TCCCACATGCTCTTCTTACAATGTGACCTTGACACTCCTCCCAT | for transposable |
| | CGAGAGGTGGGGTCTATGTCCCCTCCCCTTGAATCTGGGCGGGCCT | elements that belong |
| | TTGTGACTGCCTCGACCAATAGAATACGGCGGAAGTGACGCTGT | to MLT1G3 |
| | GTGACTTCCGAGGCTAGGTCATAAAAAGGCCATGCAGCTTCCGCC | subfamily |
| | TTGCTCTCTTGGGACGCTCGCTCTTGGAACCCAGCCGCCATGCTG | |
| | TGAGGAAGCCCAAGCCACATGGAGAGGCCACGTGTAGGTGTTCCG | |
| | GCCGACAGCCCCAGCTGAGGTCCCAGCCGACAGCCAGCATCAACC | |
| | GCCAGACATGTGAGTGAAGAAGCCTCCAGATGATTCCAGCCCCCA | |
| | GCCGTCGAGTCACCCCCAGCCTTCGAGTCTTCCCAGCTGAGGCCC | |
| | CAGACATCGTGGAGCAGAGACAAGCCATCCCCGCTGTGCCCTGTC | |
| | CGAATTCCTGACCCACAGAATCCGTGAGCATAATAAAATGGTTGT | |
| | TTTANGCCACTAAGTTTTGGGGTGGTTTGTTACGCAGCAATAGATA | |
| | ACTGGAACA | |
| 82 | TGTGGTAGATTGATTGCAAAAATGGCCNCAATTCTTCACCCCTCCC | Consensus sequence |
| | TGTATCCACGCCCTTTGCAATGTGACTTTGCAGCTCCTCCCATC | for transposable |
| | AAGAGGTGGAGTCTATTTCCCCACCCCTTGAATCTGGGCTGGCCTT | elements that belong |
| | GTGACTTGCTTTGGCCAATAGAATGCGGCGGAAGTGACGNTGTG | to MLT1H |
| | CCAGTTCCGAGCCTAGGCCTCAAGAGGCCTTGCACGCTTCCGCTCT | subfamily |
| | CTCTCTTGGAACCCTGCCACCGCCATGTGAACAAGCCCGGGCTA | |
| | GCCTGCTGGAGGATGAGAGACCACGTGGAGCAGAGCCGAGCCGTC | |
| | CCAGCTGAGGCCATCCTAGACCAGCCAGCCCCCAGCCAAGATCAG | |
| | CAGAGCCGCCTACCCGACCCGCAGCTGACCGCAGACGCATGAGTG | |
| | AGCCCAGCCGAGACCAGAAGAACCGCCCAGCTGAGCCCAGCCCA | |
| | AATTGCCGACCCACAGAATCGTGAGCTAAAATAAATGGTTGTTGTT | |
| | TTAAGCCACTAAGTTTTGGGGTGGTTTGTTACGCAGCAATAGCTA | |
| | ACTGATACA | |
| 83 | GAAATTGGTACCTAGAAGTGGGGTGCTGCCGTAACAAAAACCTAA | Consensus sequence |
| | AACATGTGGCATTGGCTTTGGGACCGGGCGGCGGGCAGAGGCTGG | for transposable |
| | AAAAGCGNCGAGGAGACTGTTAGTGGAGGCTGGAAGAGCAGTGA | elements that belong |
| | GGAAACTGCTATTGGAGGCTGGAAAAANGGCGACCCGTGTTATGT | to MLT1H-int |
| | AGTGGCGGAACANTTGGCAAAACTGTCGCCTGCGGTAACNTGGAA | subfamily |
| | GATAGAAAATGTACCTAATGAACTTGTGGATCTGGCTAAGGAGAT | |
| | TTCCAGGCAGAATGTTGAAAGTGCCAACTGGCTTCTTTTAGCTGCG | |
| | TATGATAAGGTACGGNAAGAGAGAGATGAGCTAAAGAAGGAACT | |
| | GTTCAGTTTNCAAGCAGAATTTAGAGGAAATATAGAGGANCCAGG | |
| | ACTTGCTGGGTTGGAAAATAAAACTGTTTCTCATCTCCAGTCTCTC | |
| | CAGCCGGCAAAAGATTCTCAAAGTAAGAAATGGCCTCAGGGCAAA | |
| | GATCAAATCAAGGGTGTGGCTGTAAGACCCTTTGTTAAGACCTCA | |
| | GAAAGATTTAAGGCGGTGCCTNGTAGACCCTCTCAGCTAGACAAA | |
| | AGGGCTTCTAAGAATCTTAAGGGCGTTGTCCCACAGCAGCCTGAC | |
| | ACGCAGCCCAAAGTAGAGAGAGGCCTGTCTCGAAAAGAATTGTGG | |
| | GTGTGGCTTTTGTCNAATGGAGTGGACTNNAATNNGATNCATAGG | |
| | AAACCCACAAAGTTTTTAAGAGAATTGTATTGGCAAAAGCACCGC | |
| | CAGCTTGGACTAAAAGGGACAGAGACAGTTCAAAATGAAAAGAG | |
| | GCCTCTGGGCCCCCAACTTTCTACGGGCAGGAAGCAGGCTGAGAA | |
| | AGCTACTCAGCTGCAAACACGGGCCATTTCTTATGGAAAAGGAAG | |
| | GACGNCTCAGAGGGCAGAGCCAAGAGCCCAGAGGGCGGAGCCAA | |
| | GAGCCANGGAGAACAATGGACTAGGGAACCACTCCCAGGGAGCA | |
| | GAACCGGGCCCTAATCAAGGAACATTCCCTGCCCCCGGAGTAGGG | |
| | GAACCTGGCAACATGTGCCCGGCTGGATTTCAGAATTGCTATGGA | |
| | CCAGTGACTGCTATGTGCCTCCCATTCTTCCCCTTTTTGAATGGGA | |
| | GTGTCTATTGCGGTTATCCTGTCCCTGTCTCACCATTGTATGTTGGG | |
| | TGTGTGGGGGGCAGATAACTTGTCTTTTTAGTTCACAGGTCTCCGG | |
| | ATCAAGAGGAGCCACACCCTGAGGAGCTGCACCCGAGGAGCCTC | |
| | ATCCGCATCCGGACCTGATNTAGATNACGAGATCCTGGACTTCGA | |
| | GCCTGATGCCGTAATTGGATGAGACTTTTGGGGGTCTTGGGANGG | |
| | GGGTGAGCGTATTTTGCATGTGGGAGGAATGTGAATAATTTGTGG | |
| | CCAGAGGGCAGACT | |
| 84 | TGTGGTAGATTAAAGATGGCCGCAAATTCTTTGACACTCCTCCCAT | Consensus sequence |
| | CGAGAGGTGGGGTCTATNTCCCCTCCCCTTGAATCTGGGCTGGC | for transposable |
| | CTTAGTGACTGCTTTGACCAATAGAATGCGGCGGAAGTGACGCTG | elements that belong |
| | TGCGACTTCCGAGGCTAGGTCNTAAGAAGCCTTGCAGCTTCCGCC | to MLT1H1 |
| | TNGGTCTCTTGGAACGCTCGCTCTGGGAGCCCTGAGCCGCCATGTA | subfamily |
| | AGAAGTCCGACTACCCTGAGACCGCCATGCTGGAGAGGCCACGT | |
| | GNAGGCGCTCTGGTCGACAGTCCCAGCTGAGCCCAGCCTTCCAGC | |
| | CATCCCCGCCAAGGCGCCAGACATGTGAGTGAAGCCGTCTTGGAC | |
| | CCTCCAGACCAGCCCANCCGCCAGCTGAATACCACCGAGTGACCT | |
| | CAGTCGACGCCACGTGGAGCAGAAGAACCGCCCAGCTGAGCCCTG | |
| | CCCGAATTCCTGACCCACAAAATCGTGAGANATAATAAAATGGTT | |
| | GTTGTTTTAAGCCACTAAGTTTTGGGGTAGTTTGTTACGCAGCAA | |
| | TAGATAACCGGAACA | |
| 85 | TGTGGTAGACTGTTACATTGGTGGCCCCCAATGAACCACGCCTCCC | Consensus sequence |
| | GGTATTCACGCCCTTGTGTAGTCCCCTCCCACATTGACTCTGGG | for transposable |
| | CTTGGCCATGTGACTTGCTTTGGCCAATGGGACATTAGCAAACGTG | elements that belong |
| | ATGCAAGCAGAGGCTTGANAAGCGCTTGCGCATTGGGGCTTGTC | to MLT1H2 |
| | CTCTTGGAACGCTCCCTCTTGGAANCCAGCTGCCATGCTGTGAAGA | subfamily |
| | AGCCCAGGCTAGNCTGCTGGANGATGAGAGGCCACGTGGAGAGA | |
| | GGCCCTGGAGGATGAGAGGCCATCTTGGACGTTCCAGCCCCAGCC | |
| | GAGCTCCCAGCTGAATGCAGCCACATGAGTGACCCCAGCTANACC | |
| | ACGTGGAGCAGAAGAACCGCCCAGCTGAGCCCAGCCAACCCACA | |
| | GAATCGTGAGAAATAATAAATCGTTGTTGTTTTAAGCCACTAAGTT | |
| | TTGGGGTGGTTTGTTACGCAGCAATAGATAACTGAAACA | |
| 86 | TGTGGTAGTCATTAGTGCTGTTCACCAAATATTTCCGGCTCTCTNC | Consensus sequence |
| | CTTCCGGGCACATGGTAGGATTGCACTTCCCCGCCCCCTTGAAG | for transposable |
| | TTAGGCGTGGCCATGTGACTTGCTTTGGCCAATGAAATGTGAGCGG | elements that belong |
| | AAGTGACGTGTGTCACTTCCGGGCGGAAGCTTTAAGAGCCAGTG | to MLT1I subfamily |
| | CGTGATTCGCCACGTTCCCCTTTCCCTCTGCCACGGCGACCGGCAA | |
| | TGTTCCAGATGGTGGCTGCTCCGTCAGCCTGGGTCCCGGAGTGA | |
| | GGACGACGTGGAGCAGAGCCCCCAGCCGACCCGCGATGGACATGT | |
| | AGCATGAGCGAGAAATAAACCTTTGTTGTTTTAAGCCACTGAGAT | |
| | TTGGGGGTTGTTTGTTACCGCAGCATAACCTAGCCTATCCTGACTG | |
| | ATACA | |
| 87 | TGTGGCAGAGACTGCTAGTTGTCCCCCAATATCCATTCTCCCCTTC | Consensus sequence |
| | TTCCTTAGTAATAGAACCCCCGATTTTTAGCTGGGCACATGGCC | for transposable |
| | GCCCAGAATAAAGACTACATTTCCCAGCCTCCCTTGCAGCTAGGTG | elements that belong |
| | TGGCCATGTGACTAAGTTCTGGCCAATGGGATGTAAGCGGAAGT | to MLT1J subfamily |
| | GNCGTGTGCAACTTCCGGGAAGTGTCCTTAAAGGGAGGGGGCGTG | |
| | CCCTTCTTTTCCCCTTCCTCCTTCCTGCTGNCTGGAATGCGGACG | |
| | TGATGGCTGGAGCTCNAGCAGCCATCTTGGACCATGAGGTGGAAG | |
| | CCACGTGCTGAGGATGGCGGAGCAACAAGATAGAAGGAGCCTGG | |
| | GTCCCTGATGACCGTGGAGCCGCCATACCAGCCCTGGACTGCCTAC | |
| | CTCCGGACTTCTTTTACGTGAGAGAGAAATAAACTTCTATCTTGT | |
| | TTAAGCCACTGTTATTTTGGGTTTTCTGTTACTCGCAGCCGAACCTA | |
| | ATCCTAACTGATACA | |
| 88 | GAAATTGGTACCTAGAAGTGGGGTGCTGCCATAACAAAAACCTAA | Consensus sequence |
| | AATATGTGGCATTGGCTTAGCGGTCGGGCGGCGGGCGGCGAGGAA | for transposable |
| | ACNGATATCGNAGGCTGGAAAGNTGGNGACCCNTGTTATGCAGTG | elements that belong |
| | GCAAAACATTTGGTAAAACTGTCGCCTGCGATAACTTGGAAGGCA | to MLT1J-int |
| | GACCACGTGCCTACTGAGCCTGTAGCTCTAGGGGAAGNGGTTGGA | subfamily |
| | AAAANTCAGAATGTTAGTGTGTGTTGGCTGCTNCTNGCTGCNTTT | |
| | AGCAAGGTATTACAAGAAAGAGATGAGCTCAGGAAAGAATTGGC | |
| | CGGTTTGCAAGCAGAAATGAAAGGGAATAGAGAGAGTCCAGAAA | |
| | TTTGGGGCCTTGCAGGGTTGGAAAAGCCAACTGCTTCTGNACCCCA | |
| | AACAGTAAGAGATAAGACTGAAAAAGGCTTTGAGCAACAAAGGC | |
| | CCATTAAGACTTCTCGCCAGACAAAGGGACTCAGCCCTGCGGCAA | |
| | AGATCAGATTAAGGGTGTTGCCTTCCCACCCAAGCCTATTGTTTCA | |
| | GATGGCCTCAAGGTAGCCGCCATTAAGTTGAGAGAGAGGGGATGG | |
| | GCAGAGCACAGAGGCCAGNAAATAAAAGANTAAAGCAGGCTTGA | |
| | GAACTATGTCTAGGAAAGAACTTTGGNTGTGGTTACTGGCACATG | |
| | GAACTGACTGGAAGCAAATAGATCAGAAGCCTACTAAGTTTTTGA | |
| | GGGAATTGTATTGCCAAAGAAACCACAAGCCTGGCCTGNAAAAGC | |
| | CTGTGACTGTTCGANCCCTAAAACAACCCTTGGGCCCCCAAACTTG | |
| | CACCAGCAGGAAGCGGGCTGCGAAAGCTGTGCAGCCCCCAAGGA | |
| | GGGCATACTCCCCAACGCCCACTTCAGATGTGGCCANGGAGGATA | |
| | ATGGACAAGGAAGAACCTCCCAGAGGGCGGAGCCAGGGGCCACG | |
| | GAGAACAATGGACAAGGGAGTTCCTCCCAGAGAGCAGAATCAGG | |
| | GTCTAATCAAGGAACTTCCCCCACTGCCAGGGCAGGGGGTCTTCA | |
| | CAATNCCTGCCCAGCAGGATTTCANAATTGCTATGGACCAGTGACT | |
| | GCTGTGTGTCTCCCATTCTTCCCTTTTCCGAATGGGAGTTTTTATTG | |
| | CGGTTATCCTGTCCCTGCTCCACCATTGTATATTGGGTGTGTGGGG | |
| | GGCAGATAACTTGTCTTTTAGTTCATAGGTCGCCGGACCACGAGGA | |
| | GCCACATCCGGACCTGATGGAGAGGACTGCGCATCACCCAGAGA | |
| | TCCTGGACTTTGAGCTGGATGCAGTAACTGGATGGGACTTTGGGTT | |
| | GTCTCCCTTGGGGAGGGGGTGAGTGTGTTCTATGTGTGGGAAGA | |
| | AGGGTGNAACGGATATTTGGTGGCCAGAGGGGCAGAC | |
| 89 | TGTGGCAGAGACTGGCTAGCTGTTCACCAAACCCGTTTCCTCTTCT | Consensus sequence |
| | TCCTGGGCACACAGCTAGACTACATTTCCCAGCCTCCCTTGCAG | for transposable |
| | TTAGGTGTGGCCATGTGACTGAGTTCTGGCCAATGGAATGTGAGC | elements that belong |
| | AGAAGTGATGTGTGCCACTTCCAGGCCTGGCCCATAAAAACCTCC | to MLT1J1 |
| | CATGCATNCTCCTCTCTATTCTCTCTTCCCTCTCTGCTGGCTGGATG | subfamily |
| | TCGACGCCCAGGGCGACCTTGGAAGCCACGTGTTGAAGATGGC | |
| | AGAGCCTCCGTCAGCCTGGGTCCCTGAATGACTGCGTGGAGCAGA | |
| | GCCCCCCCCNACCCCCCCAGCACATCCAATTGGACTTTACGTGAG | |
| | CGAGAAATAAACTTCTATTGTGTTAAGCCACTGAGATTTTGGGGTT | |
| | TATCTGTTACAGCAGCTAGCGTTACCTTAACTAATACA | |
| 90 | TGTGGCAGAGACTGGCTAGNTGTTCACCAAANCCCGTTTCCTTTTC | Consensus sequence |
| | CTCCTGGGCACACAGCTAGACTACATTTCCCAGCCTCCCTTGCA | for transposable |
| | GTTAGGTGNGGCCATGTGACTGAGTTCTGGCCAATGGAATGTGGG | elements that belong |
| | CGGAAGTGATGTGCGCCACTTCCAGGCCTGGCCCATAAAAACCTC | to MLT1J2 |
| | CCACGCGATCCTCCACGCTCTCTCTCTTTCCCCATCTGCCGGCTGG | subfamily |
| | ATGCAGAGGATCCAGTGGAGGACTCCGAGGCCCTAGGGGATGGC | |
| | GGAGCCACAAGATGGAAGGAGCCTGGGTCCCTGAATGACCGCGTG | |
| | GAGCAGAGCCGCCCACCCCCCAGAACCACCCGCATTGGACTGTGA | |
| | CGTGAGCGAGAAATAAACTTTTATTGTGTTAAGCCACTGAGATTTG | |
| | GGGGTTGTTTGTTACAGCAGTTAGCCTACCCTGACTAATACA | |
| 91 | TGTAGTGGACATCTGTTGTTTTTGCCTGCCCAGCATCCATTCCCCCT | Consensus sequence |
| | TCTTCTGGTAACAGCACCCCGATTTTCCTTTGGGGAACCACCC | for transposable |
| | CTCCCCCACTCTCAGTCCATGTGGTTCGGGTGGGGCTGACCCCACC | elements that belong |
| | CCTCGGCTCCAGGGGTGGGCACGTGACCCAGGCCTGGCCAATCA | to MLT1K |
| | GAGCATTCCATCCCCCTGGCCACAGTGATTGGTTCAGGGATGGGC | subfamily |
| | ACGTGACCCAAGCCGGGCCAATGAGAGTCAGCCCTGGGACTTTTG | |
| | CTGGAACTATTGGGAAAGAGANGCTCTCTTTCCGCTGGGGTTGCTA | |
| | AGCTGGNAGGATGTAAGCCTGGAGCTGCTGGCGGCCATCTTGCC | |
| | ACCACGTGGGGAGAGCCTGCCTGAGAATGAAGCCAACACAGAGG | |
| | AAAGCAGAGCCGAGAGATGGAGAGAGACAGANTCCTGATGACAT | |
| | CGTTTGAGCNCCTGGATCCAGCCGTGCCTGAAGCCAGANCTACCC | |
| | CTGGACTTTTCAGTTACGTGAGCCAATAAATTCCCTTTTTTGCTTAA | |
| | GCCAGTTTGAGTTGGGTTTCTGTCACTTGCAACCGAAAGAGTCCTG | |
| | ACTAATACA | |
| 92 | TGTGGCAGACACTGTTGGTTGCCTACCCAACAGCCATTCTCCCCTT | Consensus sequence |
| | CTTCCTTGCTAACAGAACCCCGATTTTGTTCAGGTATCNGGCGG | for transposable |
| | CCATGTGCTTCAGGGGAGGCTGGNCCCCTCCCCAGCCCCAGGGGG | elements that belong |
| | TGAATCNTGATTGGTCTAAGCCAATCATGGTAATCCCATTCCCCT | to MLT1L |
| | TGCCAGTGATTGGTTTAGGAATGGGCATGTGACNCAATTCTGGCCA | subfamily |
| | ATGAGACGTGAGGGGAAGTCTGCTGGGGGGCTTCTGGGAAAGNT | |
| | TTCCTCGCTCTTAAAAAAAGGACATAAGGAAGAGATGTCCTCTTTT | |
| | CTTGCCTCTGGACGTTGTCGTGTGNGGATGTGATGCCTGGAGCT | |
| | GCNGCAGCCATCTTGCGACCATGAGGGGACAAGCCTGAGAGGAAA | |
| | AGCCAACACGCTGAGGATGGCAGAGCGGAAAGATGGAAAGAACC | |
| | TGGGTCCTTGATGACATCGTTGAGCCGCTGAATTAACCAACCCTGG | |
| | AACCGCCCTACCTCCGGACTTCTTGTTATGTGAGATAATAAATTT | |
| | TCCTTATTGTTTAAGCCANTTTGAGTTGGGTTTTCTGTTACTTGCAG | |
| | CCGAAAGCATCCTAACTGATACA | |
| 93 | TGTACTAGACATGTCAATCACGGTGCCTGCCACGCCCACAGCCCCT | Consensus sequence |
| | TCTAAGGGAAACTGCCCCGCCCACAGCCCCTGCTGAAGGGGCAG | for transposable |
| | CCCTAGGCGGCCATGTTTTGTACCACGTGACCCTGCCCTCCCCTGG | elements that belong |
| | CCACAGCTGATTGGACCAGGGGTGGGCACCTGACCCAAGGGCAG | to MLT1M |
| | CCAATCCATAGGCTGGCCAGCGACCTATGACGTGGCCTGGCGCGA | subfamily |
| | AAAGATGAGCTGGGCCAATCAGATTCTCTCTCTCGGGAATTTGAA | |
| | CTGGGAAACACGGAGAGAATGAGGCAGTTAGCAGCGGGAGCTGA | |
| | AGCTGAAAGGATGCATAGAGAGAAGCCATGAGGTAGAGTCGGGG | |
| | CCATGATGGGCCATGTGCAAGCCGAAGTTATGAGGAAGCAGAAAC | |
| | TATGAGTAAGCAGAGGAAGCCGGTCGGTAGAGAGAAGAGAACGG | |
| | AGCAGACGCGCAGAGAGAAGCNGAGACGCGTGATGAGAGAGGGA | |
| | CCGGACGAGAGCNGCNGAGGTCCCTAGAGCTGCCCCGGTTCCGGC | |
| | CGCTTCCAGTCCCTGTTCCTGGCGNTTTCCCAGTTCCAGTTCCGGTC | |
| | CACGTGTATCCTTACAATAAACCCCCCTTTTCTTGAGNTAACTTGA | |
| | GTGAGTCTCTGTTCCTTGCAACCAAAAGAGCCTAACTAAAACA | |
| 94 | TGTAGTGGACATTTGTCATTCTTTTTGGCCGCCCAGCATCTGAACN | Consensus sequence |
| | CCCTTCCTATGTTTGGGGAATTCCCCACCTTATGAGTCCCGCCT | for transposable |
| | CCCCAAGGTAGAAGCCAGAAACTCGCTTTCCCAGCCTCCCTTGCAG | elements that belong |
| | CTAGGGCGCGGGCACGTGACCTAGGCTCCGCCAATCAGACGCAC | to MLT1N2 |
| | CCGCNCCAGACTTTGAATCGGAAGCTAGTGACGCAAGGAAGCAGG | subfamily |
| | GACCGCGCGGAATCCATTCTCTGGCGAGGGTGGCGGCAGCTGGCA | |
| | TCCAGTTTCCAGAGGCAGCAGTGGCAGNGGTTCTAGCGGCGGCGT | |
| | CCAGCGCTCATGTGTGGTGCAAGCTGCGGTGTCTGTGCCCAGCGG | |
| | CGGCAGCAGTGGTGTCCTCACCGGACCAGTTCTGCGGCGTGATTTT | |
| | GGGCATTGTTCCTGGCTGCGTAGCCTCCAAGCCTGGTTCTCCGG | |
| | CCCTCCCGGAGATTCTGTGAGCTACCCAATATCCTTTCAATAAATT | |
| | CCTTTTCTGCTTAAATTAGCCAGAGTCGGTTTCTGTTGCTTGCA | |
| | ACTAAGAACCCTGACTGATACA | |
| 95 | TGTAGTGGACACCTTTTGGGGTGTCTGCCCAGCCCCCCTTCTCTGG | Consensus sequence |
| | GAACCGCCCCCCCACCCACAGGGGTGGGCCCCCGGCAGCCATGT | for transposable |
| | TTGTACTACGTGACCCCGCCCCCCTGGCCACAGCTGATTGGACCAG | elements that belong |
| | GGGTGGACACCTGACCCAAGCTGGGCCAATCAGATTCTCTCTCC | to MLT1O |
| | CGGGAATTTGGAATTGGGACTNAGAGATNCTAGTCAGTCTCTGCT | subfamily |
| | GGTCGCTTGAACTGGGGANATGTAAACTCGGGAGCTGTGGGGCGG | |
| | CCATNTTCCGCCATGTGNACGGAGAAGCAGAGAAAGCCGGTCTGC | |
| | AGAGAGAGGAGAATGAAGCAGACGCGCAGAGAGAAGCAGAGACG | |
| | AGAGACCGGGTGGCCTCAGAGACAGAGAAAGATGGAGCCGATTCT | |
| | CTCCTGAGGCCCGGCTGCATTCCTGCCCTTGGGTTCCGTGAGACAC | |
| | CCCTGTATCCTTATAATAAATTCCCCTTTTTTGCTTAAGCTAGCTCG | |
| | AGTGGGTTTCTGTTACTTGCAACCAAAAGAGCCTTGACTAAGA | |
| | CA | |
| 96 | TGTGATGGTTAATACTGAGTGTCAACTTGATTGGATTGAAGGATAC | Consensus sequence |
| | AAAGTATTGATCCTGGGTGTGTCTGTGAGGGTGTTGCCAAAGGA | for transposable |
| | GATTAACATTTGAGTCAGTGGGCTGGGAAAGGCAGACCCACCCTT | elements that belong |
| | AATCTGGGTGGGCACNATCTAATCAGCTGCCAGCGCGGCTAGAAT | to MLT2A1 |
| | ATAAGCAGGCAGAAAAACGTGAAAAGAGAGACTGGCCTAGCCTC | subfamily |
| | CCAGCCTACATCTTTCTCCCGTGCTGGATGCTTCCTGCCCTCGAAC | |
| | ATCGGACTCCAAGTTCTTCAGTTTTGGGACTCGGACTGGCTCTCCT | |
| | TGCTCCTCAGCTTGCAGACGGCCTATTGTGGGACCTTGTGATCG | |
| | TGTGAGTTAATACTTAATAAACTCCCCTTTATATATATATNTATTCC | |
| | ATTAGTTCTGTCCCTCTAGAGAACCCTGACTAATACA | |
| 97 | TGTGATGGTTAATACTGAGTGTCAACTTGATTGGATTGAAGGATGC | Consensus sequence |
| | AAAGTATTGTTCCTGGGTGTGTCTGTGAGGGTGTTGCCAAAGGA | for transposable |
| | GATTAACATTTGAGTCAGTGGACTGGGAGAGGCAGACCCACCCTC | elements that belong |
| | AATCTGGGTGGGCACCATCTAATCAGCTGCCAGCGCGGCCAGAAT | to MLT2A2 |
| | ANAAAGCAGGCAGAAGAACGTGGAAAGACTAGACTGGCTNAGTC | subfamily |
| | TTCCGGCCTNCATCTTTCTCCCGTGCTGGATGCTTCCTGCCCTCGA | |
| | ACATCGGACTCCAAGTTCTTCAGCTTTGGGACTCGGACTGGCTTCC | |
| | TTGCTCCTCAGCTTGCAGACGGCCTATTGTGGGACCTCACCTTG | |
| | TGATCGTGTGAGTCAATACTCCTTAATAAACTCCCCTTTATATATA | |
| | CATCTATCCTATTAGTTCTGTCCCTCTAGAGAACCCTGACTAAT | |
| | ACA | |
| 98 | TGTGATGGTTAATTTTATGTGTCAACTTGACTGGGCCANGGGATGC | Consensus sequence |
| | CCAGATAGCTGGTNAAACATTATTTCTGGGTGTGTCTGTGAGGG | for transposable |
| | TGTTTCCGGAAGAGATTAGCATTTGAATCGGTAGACTGAGTAAAG | elements that belong |
| | NAGATCGCCCTCCCCAATGTGGGTGGGCATCATCCAATCCGTTGA | to MLT2B1 |
| | GGGCCTGAATAGAACAAAAAGGCGGAGGAAGGGNGAATTCGCTC | subfamily |
| | TCTCTGCTTGAGCTGGGACATCCGTCTTCTCCTGCCCTCGGACATC | |
| | GGCGCTCCTGGTTCTCGGGCCTTCGGACTCGGACTGGGACTTACAC | |
| | CATCGGCTCCCCTGGTTCTCAGGCCTTCGGACTCGGACTGGAAC | |
| | TACACCACCGGCTTTCCTGGGTCTCCAGCTTGCAGACGGCAGATCG | |
| | TGGGACTTCTCAGCCTCCATAATCGCGTGAGCCAATTCCTCATA | |
| | ATAAATCTCTTTCTATATATCTATATATATCCTATTGGTTCTGTTTC | |
| | TCTGGAGAACCCTGACTAATACA | |
| 99 | TGTGATGGTTAATTTTATGTGTCAACTTGACTGGGCTAAGGGATGC | Consensus sequence |
| | CCAGATAGCTGGTAAAACATTATTTCTGGGTGTGTCTGTGAGGG | for transposable |
| | TGTTTCCGGAAGAGATTAGCATTTGAATCAGTAGACTGAGTAAAG | elements that belong |
| | AAGATCCGCCCTCACCAATGTGGGCGGGCATCATCCAATCCGTTG | to MLT2B2 |
| | AGGGCCCGAATAGAACAAAAAGGCGGAGGAAGGGCGAATTCNCT | subfamily |
| | CTCTCTTCTTGAGCTGGGACATCCATCTTCTCCTGCCCTCGGACAT | |
| | CGGAGCTCCTGGTTCTCGGGCCTTCGGACTCCGGGACTTACACCAG | |
| | CGGCCCCCCTGGTTCTCAGGCCTTCGGACTCGGACTGAATTACA | |
| | CCACCGGCTTTCCTGGTTCTCCAGCTTGCAGACGGCANATCGTGGG | |
| | ACTTCTCGGCCTCCATAATCGCGTGAGCCAATTCCCATAATAAA | |
| | TCTCCTCTTATATATCTCTATATATCCTATTGGTTCTGTTTCTCTGG | |
| | AGAACCCTGACTAATACA | |
| 100 | TGTGATGGTTAATATTAGGTGTCAACTTGATTGGATTGAAGGATGC | Consensus sequence |
| | CNAGATAGCTGGTAAAGTATTGTTTCTGGGTGTGTCTGTGAGGG | for transposable |
| | TGTTGCCAGAGGAGATTAACATTTGAGTCAGTGGACTGGGAGAGG | elements that belong |
| | AAGACCCACCCTCANCGATGTGGGTGGGCACCATCCAATCGGCTG | to MLT2B3 |
| | CCGGCNCGGCTAGAACAAAAAGGCAGAGGAAGGGCGAATTTGCT | subfamily |
| | CTCTCTCCTGGAGCTGGGACACCCTTCTCCTCCTGCCCTCGGACAT | |
| | CAGANCTCCAGGTTCTTCGGCCTTTGGACTCTNGGACTTACACCAG | |
| | CGGTTTNCCGGGCTCTCGGGCCTTCGGCCACAGACTGAAGGCTG | |
| | CACCGTCGGCTTCCCTGCTTTTGAGGCTTTNGGACTCGGACTGAGC | |
| | CACGCTACCGGCTTCCCTGGCTCCCCAGCTTGCAGACGGCCTAT | |
| | CGTGGGACTTCNCGGCCTTCGTGATCGTGTGAGCCAATTCCCCTAA | |
| | TAAATCCCCTTTATCTATCCTATTAGTTCTGTCCCTCTGGAGAA | |
| | CCCTGACTAATACA | |
| 101 | TGTGATGGTTAATTTTATGTGTCAACTTGACTGGGCCACGGGGTGC | Consensus sequence |
| | CCAGATATTTGGTCAAACATTATTCTGGGTGTGTCTGTGAGGGT | for transposable |
| | GTTTCTGGNATGAGATTAACATTTGAATCGGTAGACTGAGTAAAG | elements that belong |
| | CAGATTGCCCTCCCTAATGTGGGTGGGCCTCATCCAATCAGTTGA | to MLT2B4 |
| | AGGCCTGAATAGAACAAAAAGGCTGACCCTCCCCCGAGTAAGAGG | subfamily |
| | GAATTCCTCCTGCCTGACTGCCTTCGAGCTGGGACATCGGTCTTT | |
| | TTCCTGCCTTCGGACTCGAACTGAAACATCGGCTCTTCCTGGGTCT | |
| | CGAGCCTGCCGGCCTTCGGACTGGAACTACACCATCGGCTCTCC | |
| | TGGTTCTCAGGCCTTCGGACTCGGACTGGAACTACACCATCGGCTC | |
| | TCCTGGGTCTCCAGCTTGCCGACTCGNNNTGCAGATCTTGGGAC | |
| | TTNTCAGCCTCCATAATCGCGTGAGCCAATTCCTTATAATAAATCT | |
| | CTCTCTTNTATATATATACCACATCCTATTGGTTCTGTTTCTCT | |
| | GGAGAACCCTAATACA | |
| 102 | TGTGATGGTTGATTTTGGGTGTCAACTTGACTGGATTAAGGGATAC | Consensus sequence |
| | CCAGATAGCTGGTAAAGCATTATTTATTCTCAATCANCGCATNA | for transposable |
| | ATTATTCTCAATGCTTCAGTAGGCACTGAGCCCGTCCCTCTTCTGCT | elements that belong |
| | GAAAGGGAAACCCAGGTGGTTTGGCATTTGATTAGAATGATTG | to MLT2B5 |
| | GGCTGCCCCAGGTGTGTCTGTGAGGGTGTTTCCGGAGGAGATTGG | subfamily |
| | CATGTGAGTCGGTGGACTGAGTGGGGAAGATCCGCCCTCAATGTG | |
| | GGCGGGCACCATCCAATCGGCTGGGGGCCCGGATGGAACAAAAA | |
| | GGCAGAGGAAGGGCGAATTCTTGCNCTCTCTCTTCCGGAGCCGGG | |
| | ACGCCCTTCTTCTCCTGCCCTTGGACGTCAGAACTCCAGGTTCTCC | |
| | GGCCTTTGGACTCCGGGACTTGCACCAGCGGCCCCCGGGCTCTCA | |
| | GGCCTTCGGCCTCGGACTGAGNNACTGNGAGTTACACCATCGGCT | |
| | TCCCTGGTTCTNAGGCCTTCGGACTTGGACTGAGCCACGCCACCG | |
| | GCTTCCCTGGNTCTCCAGCTTGCAGATGCAGNCGGCCTATCGTGGG | |
| | ACTTCTCAGCCTCCGTAATCGCGTGAGCCAATTCCCCTAATAAA | |
| | TCCCTTCTCATATATCTNTCTATATATATATATATNTNTNTATATAT | |
| | CCTATTGGTTCTGTCTCTCTGGAGAACCCTGACTAATACA | |
| 103 | TGTGATGGTTAGTTTTATGTGTCAACTTGGCTAGGCTATAGTACCC | Consensus sequence |
| | AGTTATTTAATCAAACACTAATCTAGGTGTTGCTGTGAAGGTAT | for transposable |
| | TTTGTAGATGTGGTTAACATCTACAATCAGTTGACTTTAAGTAAAG | elements that belong |
| | GAGATTACCCTCGATAATGTGGGTGGGCCTCATCCAATCAGTTG | to MLT2C1 |
| | AAGGCCTTAAGAGCAAAAACTGAGGTTTCCCGGAGAAGAAGAAAT | subfamily |
| | TCTGCCTCAAGACTGCAGCATCAACTCCTGCCTGAGTTTCCAGCC | |
| | TGCCGGCCTGCCCTACGGATTTCGGACTTGCCAGCCCCCACAATCG | |
| | CGTGAGCCAATTCCTTAAAATAAATCTCTTTCTACACACATCCT | |
| | ATTGGTTCTGTTTCTCTGGAGAACCCTGACTGATACA | |
| 104 | TGTGATGGTTAATTTTATGTGTCAACTTGGCTAGGCTATGGTGCCC | Consensus sequence |
| | AGTTGTTTGGTCAAACACTAGTCTAGATGTTGCTGTGAAGGTAT | for transposable |
| | TTTTTAGATGTGATTAACATTTACAATCAGTNGACTTTGAGTAAAG | elements that belong |
| | CAGATTACCCTCCATAATGTGGGTGGGCCTCATCCAATCAGTTG | to MLT2C2 |
| | AAGGCCTTAAGAGCAAAGACTGAGGTTTCCCGAAGAAGAAGGAAT | subfamily |
| | TCTGCCTCAAGACTGCAACATAGAAATCCTGCCTGAGTTTCCAGC | |
| | CTGCTGGCCTGCCCTGCGGATTTCGGACTCAAGACTGCAACATCAA | |
| | CTCTTACCTGAATTTCCAGCCTGCCGGCCTGCCCTACAGATTTC | |
| | GGACTTGCCAGCCCCCACAATCGCGTGAGCCAATTCCTTAAAATA | |
| | AATCTCTCTCTACACATATCCTATTGGTTCTGTTTCTCTGGAGAA | |
| | CCCTGACTAATACA | |
| 105 | TGTGATGGTTAATTTTATGTGTCAACTTGGCTAGGCCACGGTGCCC | Consensus sequence |
| | AGATATTTGGTCAAACATTANTCTGGATGTTTCTGTGAAGGTAT | for transposable |
| | TTTTTAGATGAGATTAACATTTAAATCAGTAGACTTTGAGTAAAGC | elements that belong |
| | AGATTACCCTCCATAATGTGGGTGGGCCTCATCCAATCAGTTGA | to MLT2D |
| | AGGCCTTAAGAGAAAAAGACTGANNTCCCCCGAGGAAGAGGGAA | subfamily |
| | TTCTGCCNNCAGACTGCCTTCGGACTCGAGCTGCAACATCAACTCT | |
| | TCCCTGGGTCTCCAGCCTGCCGGCCTACCCTGCAGATTTTGGACTT | |
| | GCCAGCCTCCACAATCGCGTGAGCCAATTCCTTAAAATAAATCT | |
| | CTCTCTACACACATCCTATTGGTTCTGTTTCTCTGGAGAACCCTGAC | |
| | TAATACA | |
| 106 | TGTGCTGGATTGTNCTGTTGTCAACTTGGTTAAGCTGGGAACTACG | Consensus sequence |
| | TTTCCCAGAATCCCCTTCCCTGTATGGTTCTGGGTTAGAGTTGG | for transposable |
| | CCAAAGAGGAACTTGCGCGAGATTTGGGAGGCGGAAGTGAAGCA | elements that belong |
| | GCAGCCATTACTCTCTGAAGGTCGTCGCGGTCAGACGCGGTGAGA | to MLT2E |
| | GACAGACGCAGAGGTGCCCAGCGGGTTCCAGCTTGTCCTCGCTCTC | subfamily |
| | CCCCGCTCCGCGTCCAGCTCTTCTTCCCGACTGCCGGCCCTGCTG | |
| | ACCAACAGCGGCCCCAGGCCCACCACCAGACGCTTGGCTGCGGAC | |
| | CCACAGAGGCGGTAGCCACGCAGAGGCAACAGCTTTCCATAGACC | |
| | TCTCCACNAGCTCCCCTTCATGGTCCCACTTCAGCGGCTGGACGTG | |
| | CCTGGCTTCTCAGATTTCCCCGCAAGCTCCGACTCGTCCACCCG | |
| | CGCCAGTGCTTCAGGAGGGGCTGGTTAGTGACTTTCTCTGATCCTC | |
| | CAACTCCCCCTTCCAGACCTTCACTTCCCCAGCTCCTCCCACAA | |
| | TTGTGTAAGGTCTAATTCCTATAATAAATCCCTTATCCCATAACAC | |
| | TCATAGTGGCTCTGCTTCCCTGACTGAACCCTGACTGATACA | |
| 107 | TGTGGTGGCTTTGTAATGTGTCAACTTGGCTAGGCTGAACTACGTT | Consensus sequence |
| | TCCCAGAATTCCCTTCCCTGTATGTTTCCGGTTAGGGTGGGCCA | for transposable |
| | CAAGAGANATTCTGCGCGAGATTTGGAGGGCGGAAGTGAAGCAGC | elements that belong |
| | AGCCATNTTGTTTTTNATGCTCGGAAGGTCGGNGCAGGGNCCGCA | to MLT2F subfamily |
| | GGCGCTNTTGCAGCTCACGCACGTTGTCGCTNATCTGCTGGCTCAC | |
| | CTCGTTGGCGTGGGGCAGCAGCCGGGCCTGCAGCTGCTCCACCT | |
| | TCCCCTGGATCCTCCTTCAGCTTCTCCGACTCCTGGGCCAGGTGTG | |
| | TGTTTAGCTCCGTGACGAAGGGCGCCAGCTTCTCCTGCAGGACA | |
| | CCCGCATCATCAAGGTCGGAGGCAGCGAGAGACTGACACGGGTTC | |
| | CAGTCCGTCCTCGTGGGTTCCAGCTCGTGCTCGTGGGTTCCAGCT | |
| | TGTCCTTGCTCTCCCCCACTTCACATCCATCTTCCCTTCCCGACTGC | |
| | CTGCCCTGCGGACTTCAAGCTCCAGCATCAGACGCAAAGACAA | |
| | CAGCCTTACAGAGACTGCTTAACCAGCTCCCACAATTGCGTAAGGT | |
| | CAAATCCCTGTAACAAATCCCTTATTATATATATCTCCTAGTGG | |
| | TTCTGCTTCTCTGATTGAACCCTGACTGATACA | |
| 108 | GAAAATTGGTACCGAGGAGTGGGGCATTGCTATAAAGATACCTGA | Consensus sequence |
| | AAATGTGGAAGCGGCTTTGGAACTGGGTAACGGGCAGAGGTTGGA | for transposable |
| | AGAGTTTGGAGGGCTCAGAAGAAGACAGGAAGATGAGGGAAAGT | elements that belong |
| | TTGGAACTTCTTAGAGACTGGTTAAATGGTTGTGACCAAAATGCTG | to MST-int |
| | ATAGTGATATGGACAGTGAAGNCCAGGCTGACGAGGTCTCAGATG | subfamily |
| | GAAATGAGGAACTTATTGGGAACTGGAGCAAAGGTCACCCNTGTT | |
| | ATGCCTTAGCAAAGAACTTGGCTGCATTGTGTCCATGCCCTAGGGA | |
| | TCTGTGGAAGTTTGAACTTAAGAGTGATGACTTAGGGTATCTGG | |
| | CGGAAGAAATTTCTAAGCAGCAAAGCGTTCAAGATGTGGCCTGGC | |
| | TGCTTCTAACAGCCTACGNTCAGATGCGGGAGCAAAGAAATGACT | |
| | TAAAGTTGGAACTTATATTTAAAAGGGAAGCAGAGCGTAAAAGTT | |
| | TGGAAAATTTGCAGCCTGGCCATGTGGCAGAAAAGAAAAANCCAT | |
| | TTTCAGGAGAGGAATTCAAGCAGGCTGCGGAGCAACCACTTGCTA | |
| | GAGAGATTTGCATGACTAAAAGGGAGCCAAGTGCTAATANCCAAG | |
| | ACAATGGGAAAAAGGCCTCGAAGGCATTTCAGAGATCTTCGAGGC | |
| | AGCCCCTCCCATCACAGGCCCAGAGGCCTAGGAGGAAAGAATGGT | |
| | TTCGTGGGCCAGGCCCAGGGCCCCGCTGCCCTGCGCAGCCTCGGG | |
| | ACACTGCTCCCCGCATCCCGGCCGCTCCGGCTCCAGCCGCGGCTC | |
| | AAAGGGCCCCAGGTACAGCTCGGGCCGCCGCTTCGGAGGGCGCAA | |
| | GCCATAAGCCTTGGCGGCTTCCACGTGGTGTTAAGCCTGCAGGCG | |
| | CACAGAATGCAAGAGTGAAGGAGGCTTGGCAGCCTCCGCCTAGAT | |
| | TTCAGAGGATGTATGGGAAAGCCTGGGTGCCCAGGCAGAAGCCTG | |
| | CTGCAGGGGCGGAGCCCTCACAGAGAACCTCTACTAGGGCAGTGC | |
| | CGAGGGGAAATGTGGGGTTGGAGCCCCCACACAGAGTCCCCACCG | |
| | GGGCACTGCCTAGTGGAGCTGTGGGAAGGGGGCCACCGCCCTCCA | |
| | GACCCCAGAATGGTAGANCCACCGGCAGCTTGCACCCTGAGCCTG | |
| | GAAAAGCCGCAGGCACTCAACTCCAACCCGTGAGAGCAGCCACGG | |
| | GGGCTGNACCCTGCAAAGCCACAGGGGCGGAGCTGCCCAAGGCCT | |
| | TGGGAGCCCACCCCTTGCACCAGTGTGCCCTGGATGCGGGACATG | |
| | GAGTCAAAGGAGATTATTTTGGAGCTTTAAGATTTAATGACTGCC | |
| | CTGCTGGGTTTCGGACTTGCGTGGGGCCTGTAGCCCCTTTCTTTTG | |
| | GCCGATTTCTCCCTTTTGGAATGGGAATGTTTACCCAATGCCTG | |
| | TACCNCCATTGTATCTTGGAAGTAAATAACTTGTTTTTGATTTTACA | |
| | GGCTCATAGGTGGAAGGAACTTGCCTTGTCTCAGATGAGACTT | |
| | TGGACTTTGGACTTTTGAGTTAATGCTGGAATGAGTTAAGACTTTG | |
| | GGGGACTATTGGGAAGGCATGATTGTATTTTGCAATGTGAGAAG | |
| | GACATGAGATTTGGGGGGGCCAGGGGCGGAA | |
| 109 | TGATATGGTTTGGATCTGTGTCCCCGCCCAAATCTCATGTCGAATT | Consensus sequence |
| | GTAATCCCCAGTGTTGGAGGNGGGGCCTGGTGGGAGGTGATTGG | for transposable |
| | ATCATGGGGGCGGATTTCTCATGANCGGTTTAGCACCATCCCCTTG | elements that belong |
| | GTGCTGTTCTCGTGATAGTGAGTGAGTTCTCACGAGATCTGGTT | to MSTA subfamily |
| | GTTTAAAAGTGTGTGGCACCTCCCCCCTCGCTCTCTCTTNCTCCTGC | |
| | TCCGGCCATGTGACGTGCCTGCTTCCCCTTCGCCTTCCGCCAT | |
| | GATTGTAAGTTTCCTGAGGCCTCCCCAGAAGCCGANNAGATGCCA | |
| | NCGCCATGCTTCCTGTACAGCCTGCGGAACCGTGAGCCAATTAAA | |
| | CCTCTTTTCTTTATAAATTACCCAGTCTCAGGTATTTCTTTATAGCA | |
| | GTGCGAGAACGGACTAATACA | |
| 110 | TGCTATGGTTTGGATGTTTGTCCCCGCCAAAACTCATGTTGAAATT | Consensus sequence |
| | TGATCCCCAATGTGGCAGTGTTGGAGGTGGGGCCTAGTGGGAGG | for transposable |
| | TGTTTGGGTCATGGGGGCGGATCCCTCATGAATAGATTAATGCCCT | elements that belong |
| | CCCTCGNGGTGGGNGTGAGTGAGTTCTCGCTCTNNCGCGGGAAT | to MSTA1 |
| | GGATTAGTTCCCGCGAGAGCGGGTTGTTAAAAAGAGTCTGGCGNC | subfamily |
| | TCCCTCCTCTCTCTCTCTTGCTTGCTTCCTCTCTCGCCATGTGAT | |
| | CTCTGCACACGCCCGCTCCCCTTCCNCTTCACTTTCCGCCATGAGT | |
| | NGAAGCAGCCTGAGGCCCTCACCAGATGCAGCTGCCCGNACCNT | |
| | GCTTTTTNNCCAGCCACCAGAATCGTGAGCCAAATAAACCTCTTTT | |
| | CTTTATAAATTACCCAGCCTCAGGTATTCCGTTATAGCAACACA | |
| | AAACGGACTAAGACA | |
| 111 | TGATATGGTTTGGATGTTTGTCCCCTCCAAATCTCATGTTGAAATG | Consensus sequence |
| | TGATCCCCAGTGTTGGAGGTGGGGCCTGGTGGGAGGTGTTTGGG | for transposable |
| | TCATGGGGGCGGATCCCTCATGAATGGCTTGGCGCCGTCCCCNTGG | elements that belong |
| | TGATGAGTGAGTTCTCGCTCTGTTAGTTCACGCGAGATCTGGTT | to MSTB subfamily |
| | GTTTAAAAGAGTNTGGCACCTCCCCCCTCTCTCTCTTGCTCCCGCTC | |
| | TCGCCATGTGACGCGCCTGCTCCCCCTTCGCCTTCCGCCATGA | |
| | TTGNAAGCTTCCTGAGGCCTCACCAGAAGCCGAGCAGATGCCGGC | |
| | GCCATGCTTCCTGTACAGCCTGCAGAACCGTGAGCCAANTAAACC | |
| | TCTTTTCTTTATAAATTACCCAGCCTCAGGTATTCCTTTATAGCAAC | |
| | GCAAGAACGGACTAACACA | |
| 112 | TGCTATAGTTTGGATGTTTGTCCCCTCCAAACCTCATGTTGAAATTT | Consensus sequence |
| | GATCCCCAGTGTTGGAGGTGGGGCCTAATGGGAGGTGTTTGGG | for transposable |
| | TCATGGGGGCGGATCCCTCATGAATGGNTTGGTGCCCTCCCTCGGG | elements that belong |
| | GTGGGGATGAGTGAGTTCTCGCTCTATTAGTTCCCGCGAGAGCT | to MSTB1 |
| | GGTTGTTAAAAAGAGCCTGGCACCTCCCTCCTCTCTCTCTTGCTTCC | subfamily |
| | TCTCTCGCCATGTGATCTCTGCACACGCCGGCTCCCCTTCGCC | |
| | TTCCGCCATGAGTGGAAGCAGCCTGAGGCCCTCACCAGAAGCAGA | |
| | TGCTGGCGCCATGCTTCTTGTACAGCCTGCAGAACCGTGAGCCAA | |
| | ATAAACCTCTTTTCTTTATAAATTACCCAGCCTCAGGTATTCCTTTA | |
| | TAGCAACACAAAACGGACTAAGACA | |
| 113 | TGCTATGGTTTGGATATGGTTTGTTTGTCCCCACCAAAACTCATGTT | Consensus sequence |
| | GAAATTTGATCCCCAATGTGGCGGTGTTGGGAGGTGGGGCCTA | for transposable |
| | GTGGGAGGTGTTTGGGTCATGGGGGCGGATCCCTCATGAATGGCT | elements that belong |
| | TGGTGCCGTTCTCGCGGTAGTGAGTGAGTGAGTTCTCGCTCTCGC | to MSTB2 |
| | GAGACTGGATTAGTTCTCGCGGGAATGGATTAGTTCCCGCGAGAG | subfamily |
| | TGGGTTGTTATAAAGCCAGGACGCCCCTCGGGTTTTGCCTCTTCG | |
| | CACGTGTCCGCTTCCCCTTTGACCTTCTCCGCCATGTTNTGACGCA | |
| | GCACGAAAGCCCTCACCAGAAGCCAGGGCCATGCCCTTGAACTT | |
| | CCCAGCCTGCAGAACCGTGAGCTAAATAAACCTCTTTTCTTTATAA | |
| | ATTACCCAGTCTCAGGTATTCTGTTATAGCAACACAAAACGGAC | |
| | TAAGACA | |
| 114 | TGCTATGGTTTGAATGTTTGTCCCCTCCAAAACTCATGTTGAAACT | Consensus sequence |
| | TAATCCCCAATGTGGCAGTATTGAGAGGTGGGGCCTTTAAGAGG | for transposable |
| | TGATTGGGTCATGAGGGCTCTGCCCTCATGAATGGATTAATCCATT | elements that belong |
| | CATGGATTAATGGATTAATGGATTAATGGGTTATCATGGGAGTG | to MSTC subfamily |
| | GGACTGGTGGCTTTATAAGAAGAGGAAGAGAGACCTGAGCTAGCA | |
| | CGCTCAGCCCCCTCGCCATGTGATGCCCTGCGCCGCCTCGGGACT | |
| | CTGCAGAGAGTCCCCACCAGCAAGAAGGCCCTCACCAGATGCGGC | |
| | CCCTCGACCTTGGACTTCTCAGCCTCCANAACTGTAAGAAATAAA | |
| | TTCCTTTTCTTTATAAATTACCCAGTTTCAGGTATTCTGTTATAAGC | |
| | AACAGAAAACGGACTAAGACA | |
| 115 | TGCTATGGTTTGAATGTGTCCCCCAAAGTTCATGTGTTGGAAACTT | Consensus sequence |
| | AATCCCCAATGCAACAGTGTTGGGAGGTGGGGCCTNNTAAGAGG | for transposable |
| | TGATTAGGTCATGAGGGCTCCGCCCTCATGAATGGATTAATGCCGT | elements that belong |
| | TATCGCGGGAGTGGGTTAGTTATCGCGGGAGTGGGTTCGTTATA | to MSTD subfamily |
| | AAAGNAAGTTCGGCCCCCTTTTGCTCTCTCTCTCTCGCTCTCTTGCC | |
| | CTTCCGCCTTCCGCCATGGGATGACGCAGCAAGAAGGCCCTCG | |
| | CCAGATGCCGGCNCCTCGATCTTGGACTTCCCAGCCTCCAGAACCG | |
| | TGAGCCAAATAAATTTCTGTTCTTTATAAATTACCCAGTCTGTG | |
| | GTATTCTGTTATAGCAGCACAAAACGGACTAAGACA | |
| 116 | GTAAATTGGTACCAGTAGAGTGGGGCGCTGCTGAAAAGATACCCG | Consensus sequence |
| | AAAATGTGGAAGCGACTTTGGAACTGGGTAACAGGCAGAGGTTGG | for transposable |
| | AACAGTTTGGAGGGCTCAGAAGAAGACAGGAAAATGTGGGAAAG | elements that belong |
| | TTTGGAACTTCCTAGAGACTTGTTGAATGGCTTTGACCAAAATGCT | to THE1-int |
| | GATAGTGATATGGACAATAAAGTCCAGGCTGAGGTGGTCTCAGAT | subfamily |
| | GGAGATGAGGAACTTGTTGGGAACTGGAGCAAAGGTGACTCTTGT | |
| | TATGTTTTAGCAAAGAGACTGGCGGCATTTTGCCCCTGCCCTAGAG | |
| | ATTTGTGGAACTTTGAACTTGAGAGAGATGATTTAGGGTATCTG | |
| | GCGGAAGAAATTTCTAAGCAGCAAAGCATTCAAGAGGTGACTTGG | |
| | GTGCTGTTAAAGGCATTCAGTTTTANAAGGGAAGCAGAGCATAAA | |
| | AGTTCGGAAAATTTGCAGCCTGACGATGCGATAGAAAAGAAAANC | |
| | CCATTTTCTGAGGAGAAATTCAAGCCGGCTGCAGAAATTTGCATA | |
| | AGTAACGAGGAGCCGAATGTTAATCCCCAAGACAATGGGGAAAAT | |
| | GTCTCCAGGGCATGTCAGAGGCCTTCGCGGCAGCCCCTCCCATCA | |
| | CAGGCCCGGAGGCCTAGGAGGAAAAAATGGTTTCGTGGGCCGGGC | |
| | CCAGGGTCCCCGTGCTGTGTGCAGCCTAGGGACTTGGTGCCCTGC | |
| | GTCCCAGCCGCTCCAGCCGTGGCTGAAAGGGGCCAACGTACAGCT | |
| | CGGGCCGTGGCTTCAGAGGGTGCAAGCCCCAAGCCTTGGCAGCTT | |
| | CCACGTGGTGTTGAGCCTGCGGGTGCACAGAAGTCAAGAATTGAG | |
| | GTTTGGGAACCTCCGCCTAGATTTCAGAGGATGTATGGAAACGCC | |
| | TGGATGCCCAGGCAGAAGTTTGCTGCAGGGGCGGGGCCCTCATGG | |
| | AGAACCTCTGCTAGGGCAGTGCGGAAGGGAAATGTGGGGTCGGAG | |
| | CCCCCACACAGAGTCCCTACTGGGGCACCGCCTAGTGGAGCTGTG | |
| | AGAAGAGGGCCACCGTCCTCCAGACCCCAGAATGGTAGATCCACC | |
| | GACAGCTTGCACCGTGCGCCTGGAAAAGCCGCAGACACTCAACGC | |
| | CAGCCCGTGAAAGCAGCCGGGAGGGAGGCTGTACCCTGCAAAGCC | |
| | ACAGGGGCGGAGCTGCCCAAGACCATGGGAACCCACCTCTTGCAT | |
| | CAGCGTGACCTGGATGTGAGACATGGAGTCAAAGGAGATCATTTT | |
| | GGAGCTTTAAGATTTGACTGCCCCGCTGGATTTCGGACTTGCATGG | |
| | GGCCTGTAGCCCCTTTGTTTTGGCCAATTTCTCCCATTTGGAAT | |
| | GGCTGTATTTACCCAATGCCTGTACCCCCATTGTATCTAGGAAGTA | |
| | ACTAACTTGCTTTTGATTTTACAGGCTCATAGGCGGAAGGGACT | |
| | TGCCTTGTCTCAGATGAGACTTTGGACTGTGGACTTTTGAGTTAAT | |
| | GCTGAAATGAGTTAAGACTTTGGGGGACTGTTGGGAAGGCATGA | |
| | TTGGTTTTGAAATGTGAGGACATGAGATTTGGGAGGGGCCAGGGG | |
| | CGGAA | |
| 117 | TGATATGGTTTGGCTGTGTCCCCACCCAAATCTCAACTTGAATTGT | Consensus sequence |
| | ATCTCCCAGAATTCCCACGTGTTGTGGGAGGGACCCAGGGGGAG | for transposable |
| | GTAATTGAATCATGGGGGCCGGTCTTTCCCGTGCTATTCTCGTGAT | elements that belong |
| | AGTGAATAAGTCTCACGAGATCTGATGGGTTTATCAGGGGTTTC | to THE1A subfamily |
| | CGCTTTTGCTTCTTCCTCATTTTCCTCTTGCCGCCGCCATGTAAGAA | |
| | GTGCCTTTCGCCTCCCGCCATGATTCTGAGGCCTCCCCAGCCA | |
| | TGTGGAACTGTAAGTCCAATTAAACCTCTTTTTCTTCCCAGTCTCG | |
| | GGTATGTCTTTATCAGCAGCGTGAAAACGGACTAATACA | |
| 118 | TGATATGGTTTGGCTGTGTCCCCACCCAAATCTCATCTTGAATTGT | Consensus sequence |
| | AGCTCCCATAATTCCCACGTGTCGTGGGAGGGACCCGGTGGGAG | for transposable |
| | GTAATTGAATCATGGGGGCGGGTCTTTCCCGTGCTGTTCTCGTGAT | elements that belong |
| | AGTGAATAAGTCTCACGAGATCTGATGGTTTTATAAAGGGGAGT | to THE1B subfamily |
| | TCCCCTGCACANGCTCTCTTGCCTGCCGCCATGTAAGACGTGNCTT | |
| | TGCTCCTCCTTCGCCTTCCGCCATGATTGTGAGGCCTCCCCAGC | |
| | CACGTGGAACTGTGAGTCCATTAAACCTCTTTCCTTTATAAATTAC | |
| | CCAGTCTCGGGTATGTCTTTATCAGCAGCGTGAAAACGGACTAA | |
| | TACA | |
| 119 | TGATATGGTTTGGCTGTGTCCCCACCCAAATCTCATCTTGAATTGT | Consensus sequence |
| | AGTTCCCATAATCCCCACGTGTCGTGGGAGGGACCCGGTGGGAG | for transposable |
| | GTAATTGAATCATGGGGGCGGTTACCCCCATGCTGTTCTCGTGATA | elements that belong |
| | GTGAGTGAGTTCTCACGAGATCTGATGGTTTTATAAGGGGCTTT | to THE1C subfamily |
| | TCCCCCTTCGCTCGGCACTTCTCCTTCCTGCCGCCATGTGAAGAAG | |
| | GACGTGTTTGCTTCCCCTTCCGCCATGATTGTAAGTTTCCTGAG | |
| | GCCTCCCCAGCCATGCNGAACTGTGAGTCAATTAAACCTCTTTCCT | |
| | TTATAAATTACCCAGTCTCGGGTATGTCTTTATTAGCAGCGTGA | |
| | GAACGGACTAATACA | |
| 120 | TGATATGGTTTGGCTGTGTCCCCACCCAAANTCTCATCTCGAATTG | Consensus sequence |
| | TAATCCCCATAATCCCCACGTGTCGAGGGAGGGACCTGGTGGAG | for transposable |
| | GTGATTGGATCATGGGGGCGGTTTCCCCCATGCTGTTCTCGTGATA | elements that belong |
| | GTGAGTGAGTTCTCACGAGATCTGATGGTTTTATAAGTGTCTGG | to THE1D subfamily |
| | CAGNTTTCCCCTGCNCTCACNCTTCTCTCTCCTGCCGCCNTGTGAA | |
| | GAAGGTGCTTGCTTCCCCTTCGCCTTCCGCCATGATTGTAAGTT | |
| | TCCTGAGGCCTCCCCAGCCATGCGGAACTGTGAGTCAATTAAACCT | |
| | CTTTCCTTTATAAATTACCCAGTCTCGGGTAGTTCTTTATAGCA | |
| | GTGTGAAAACGGACTAATACA | |
| 121 | GAAAATTGGTACCGGGAGAGTGGGGCACTGCTATAAAGATACCTG | Consensus sequence |
| | AAAATGTGGAAGCGACTTTGGAACTGGGTAACGGGCAGAGGTTGG | for transposable |
| | AACAGTTTGGAGGGCTCAGAAGAAGACAGGAAGATGTGGGAAAG | elements that belong |
| | TTTGGAACTTCCTAGAGACTTGTTGAATGGTTTTGACCAAAATGCT | to THE1D-int |
| | GATAGTGATATGGACAATGAAGTCCAGGCTGAGGTGGTCTCAGAT | subfamily |
| | GGAGATGAGGAACTTATTGGGAACTGGAGTAAAGGTCACTCTTGC | |
| | TATGCTTTAGCAAAGAGACTGGCGGCATTTTGCCCCTGCCCTAGAG | |
| | ATCTGTGGAACTTTGAACTTGAGAGAGATGATTTAGGGTATCTG | |
| | GCGGAAGAAATTTCTAAGCAGCAAAGCATTCAAGATGTGACCTGG | |
| | CTGCTTCTAAAAGCGTACGGTCATATGCGTTCACAAAGAGATGGT | |
| | CTGAAATTGGAACTTATGTTTAAAAGGGAAGCAGAGCATAAAAGT | |
| | TTGGAAAATTTGCAGCCTGACCATGCGGTAGAAAAGAAAAACCCA | |
| | TTTTCTGGGGAGAAATTCAAGCCGGCTGCAGAAATTTGCATAAGT | |
| | AACGAGGAGCCGAATGTTAATAGCCAAGACAATGGGGAAAATGTC | |
| | TCCAGGGCATNTCAGAGACCTTCGCGGCAGCCCCTCCCATCACAG | |
| | GCCCGGAGGCCTAGGAGGGAAAAATGGTTTCGTGGGCCGGGCCCA | |
| | GGGCCCCGCTGCTCTGTGCAGCCTCGGGACATGGCGCCCTGCGTCC | |
| | CAGCCGCTCCAGCTCCAGCCGTGGCTAAAAGGGGCCAAGGTACA | |
| | GCTCGGGCCGTTGCTTCAGAGGGTGCAAGCCCCAAGCCTTGGCGG | |
| | CTTCCACGTGGTGTTGGGCCTGCGGGTGCGCAGAAGNCAAGAGTT | |
| | GAGGTTTGGGAACCTCCGCCTAGATTTCAGAGGATGTATGGAAAC | |
| | GCCTGGATGTCCAGGCAGAAGTCTGCTGCAGGGGCGGAGCCCTCA | |
| | TGGAGAACCTCTGCTAGGGCAGTGCGGAGGGGAAATGTGGGGTTG | |
| | GAGCCCCCACACAGAGTCCCCACTGGGGCACTGCCTAGTGGAGCT | |
| | GTGAGAAGAGGGCCACCGTCCTCCAGACCCCAGAATGGTAGATCC | |
| | ACCGACAGCTTGCACCGTGCGCCTGGAAAAGCCGCAGGCACTCAA | |
| | CGCCAGCCCGTGAAAGCAGCCGCGGGGGCTGTACCCTGCAGAGCC | |
| | ACAGGGGCGGAGCTGCCCAAGGCCNTGGGAGCCCACCCCTTGCAT | |
| | CAGCGTGNCCTGGATGTGAGACATGGAGTCAAAGGAGATTATTTT | |
| | GGAGCTTTAAGATTTAATGACTGCCCTGCTGGGTTTCGGACTTGC | |
| | ATGGGGCCTGTAGCCCCTTTGTTTTGGCCAATTTCTCCCATTTGGA | |
| | ATGGGAGCATTTACCCAATGCCTGTACCCCCATTGTATCTTGGA | |
| | AGTAACTAACTTGCTTTTGATTTTACAGGCTCATAGGCGGAAGGGA | |
| | CTTGCCTTGTCTCAGATGAGACTTTGGACTTGGACTTTTGAGTT | |
| | AATGCTGGAATGAGTTAAGACTTTGGGGGACTGTTGGGAAGGCAT | |
| | GATTGTGTTTTGAAATGTGAGAAGGACATGAGATTTGGGAGGGGC | |
| | CAGGGGCGGAA | |

TABLE 2
Genomic positions of a list of integrants that belong to LTR16C subfamily

| Chr | Strand | End | Strand | Chr | Start | End | Strand | Chr | Start | End | Strand |
|---|---|---|---|---|---|---|---|---|---|---|---|
| chr1 | 40736 | 40878 | − | chr19 | 6988164 | 6988399 | + | chr4 | 24198639 | 24198810 | − |
| chr1 | 836414 | 836851 | + | chr19 | 7641434 | 7641674 | − | chr4 | 24356970 | 24357276 | + |
| chr1 | 2223082 | 2223263 | + | chr19 | 7739334 | 7739560 | − | chr4 | 24477337 | 24477781 | − |
| chr1 | 2223722 | 2223767 | + | chr19 | 7771351 | 7771570 | + | chr4 | 24507529 | 24507818 | − |
| chr1 | 4285609 | 4285801 | − | chr19 | 7883410 | 7883510 | + | chr4 | 24666854 | 24667206 | + |
| chr1 | 5768693 | 5768829 | + | chr19 | 8545814 | 8546056 | + | chr4 | 24978953 | 24979409 | − |
| chr1 | 6941475 | 6941619 | − | chr19 | 8546979 | 8547015 | + | chr4 | 25942543 | 25942804 | + |
| chr1 | 7245757 | 7246110 | − | chr19 | 8547621 | 8547740 | + | chr4 | 25968344 | 25968750 | + |
| chr1 | 9520553 | 9520801 | + | chr19 | 9864954 | 9865039 | + | chr4 | 26023780 | 26024006 | − |
| chr1 | 10869022 | 10869253 | − | chr19 | 9962989 | 9963205 | + | chr4 | 26059494 | 26059885 | − |
| chr1 | 11580502 | 11580771 | + | chr19 | 11279754 | 11279861 | + | chr4 | 26060271 | 26060358 | − |
| chr1 | 11850603 | 11850689 | − | chr19 | 11300179 | 11300421 | − | chr4 | 26577564 | 26577795 | − |
| chr1 | 11851949 | 11852186 | − | chr19 | 11300451 | 11300535 | − | chr4 | 26809534 | 26809734 | − |
| chr1 | 11852489 | 11852517 | − | chr19 | 13247865 | 13248184 | + | chr4 | 26810004 | 26810347 | + |
| chr1 | 11852835 | 11853011 | − | chr19 | 13305001 | 13305406 | − | chr4 | 27131103 | 27131492 | − |
| chr1 | 12090793 | 12090823 | − | chr19 | 13354604 | 13354642 | − | chr4 | 27223481 | 27223803 | − |
| chr1 | 12091215 | 12091514 | − | chr19 | 13355443 | 13355646 | − | chr4 | 29133476 | 29133546 | + |
| chr1 | 14415588 | 14415858 | + | chr19 | 13364500 | 13364625 | + | chr4 | 29134050 | 29134081 | + |
| chr1 | 14435349 | 14435644 | + | chr19 | 13364940 | 13365137 | + | chr4 | 30217557 | 30217654 | + |
| chr1 | 14445656 | 14445848 | − | chr19 | 13373428 | 13373717 | + | chr4 | 30217667 | 30217735 | + |
| chr1 | 14685052 | 14685515 | − | chr19 | 13439039 | 13439108 | + | chr4 | 30217775 | 30217899 | + |
| chr1 | 14765980 | 14766276 | − | chr19 | 13439352 | 13439386 | + | chr4 | 30257958 | 30258185 | − |
| chr1 | 14833295 | 14833561 | − | chr19 | 13442130 | 13442455 | − | chr4 | 30677052 | 30677163 | − |
| chr1 | 17003049 | 17003267 | + | chr19 | 13526521 | 13526926 | + | chr4 | 30679451 | 30679731 | − |
| chr1 | 17295845 | 17296296 | + | chr19 | 13559527 | 13559920 | − | chr4 | 31918347 | 31918561 | + |
| chr1 | 17703050 | 17703247 | − | chr19 | 13560298 | 13560627 | + | chr4 | 32178064 | 32178285 | + |
| chr1 | 17843620 | 17843770 | + | chr19 | 13617957 | 13618094 | + | chr4 | 32788058 | 32788194 | − |
| chr1 | 18061544 | 18062005 | + | chr19 | 13643514 | 13643968 | − | chr4 | 32789123 | 32789222 | − |
| chr1 | 18139728 | 18139957 | + | chr19 | 13647368 | 13647426 | − | chr4 | 33408324 | 33408616 | + |
| chr1 | 18140224 | 18140456 | + | chr19 | 13693801 | 13694258 | − | chr4 | 35974084 | 35974442 | − |
| chr1 | 18174525 | 18174900 | + | chr19 | 13696806 | 13697193 | − | chr4 | 36552845 | 36553253 | + |
| chr1 | 18261782 | 18261984 | − | chr19 | 13705172 | 13705361 | + | chr4 | 37714389 | 37714608 | − |
| chr1 | 18262151 | 18262280 | − | chr19 | 13941426 | 13941516 | − | chr4 | 37714690 | 37714800 | − |
| chr1 | 18295699 | 18295892 | + | chr19 | 14048480 | 14048917 | + | chr4 | 38795627 | 38796011 | − |
| chr1 | 18296122 | 18296253 | + | chr19 | 14237316 | 14237697 | − | chr4 | 40650182 | 40650427 | + |
| chr1 | 18387620 | 18387852 | − | chr19 | 14306839 | 14306962 | + | chr4 | 42927819 | 42928105 | − |
| chr1 | 18404435 | 18404551 | + | chr19 | 14307274 | 14307463 | + | chr4 | 43352020 | 43352437 | + |
| chr1 | 18405020 | 18405277 | + | chr19 | 14307847 | 14307915 | + | chr4 | 44524156 | 44524624 | − |
| chr1 | 18490539 | 18490722 | + | chr19 | 14339836 | 14340105 | + | chr4 | 46345338 | 46345753 | + |
| chr1 | 18499017 | 18499144 | − | chr19 | 14345474 | 14345733 | + | chr4 | 46944311 | 46944553 | + |
| chr1 | 18499927 | 18500136 | − | chr19 | 14615301 | 14615428 | + | chr4 | 47732442 | 47732565 | + |
| chr1 | 18976205 | 18976553 | − | chr19 | 14615925 | 14615966 | + | chr4 | 47732863 | 47732981 | + |
| chr1 | 20359630 | 20359650 | − | chr19 | 14616270 | 14616379 | + | chr4 | 48094525 | 48094824 | − |
| chr1 | 20359762 | 20360140 | − | chr19 | 14626574 | 14627049 | − | chr4 | 52134200 | 52134677 | + |
| chr1 | 20360432 | 20360525 | − | chr19 | 14648333 | 14648555 | + | chr4 | 52570059 | 52570341 | − |
| chr1 | 21582945 | 21583283 | − | chr19 | 14650762 | 14650848 | + | chr4 | 52918778 | 52918830 | − |
| chr1 | 21858681 | 21859013 | + | chr19 | 14662276 | 14662393 | − | chr4 | 52921810 | 52922140 | + |
| chr1 | 21966580 | 21966789 | + | chr19 | 15078047 | 15078125 | + | chr4 | 53946641 | 53946926 | − |
| chr1 | 22230295 | 22230598 | − | chr19 | 15078203 | 15078486 | + | chr4 | 54553344 | 54553778 | − |
| chr1 | 22314494 | 22314606 | − | chr19 | 15101485 | 15101748 | − | chr4 | 55626964 | 55627087 | − |
| chr1 | 22315150 | 22315514 | − | chr19 | 15142605 | 15143000 | − | chr4 | 55657597 | 55657823 | − |
| chr1 | 23409935 | 23410131 | + | chr19 | 15153992 | 15154444 | − | chr4 | 57112263 | 57112416 | − |
| chr1 | 24153209 | 24153434 | + | chr19 | 15157354 | 15157691 | − | chr4 | 57237003 | 57237110 | − |
| chr1 | 24157955 | 24158368 | − | chr19 | 15467524 | 15467785 | + | chr4 | 57730587 | 57730913 | + |
| chr1 | 24973238 | 24973612 | − | chr19 | 15467773 | 15467838 | + | chr4 | 57731484 | 57731604 | + |
| chr1 | 25040560 | 25040877 | + | chr19 | 15467924 | 15467940 | + | chr4 | 58163159 | 58163562 | − |
| chr1 | 25063721 | 25064178 | − | chr19 | 15468539 | 15468618 | + | chr4 | 58556311 | 58556392 | + |
| chr1 | 25524398 | 25524799 | − | chr19 | 15523674 | 15523768 | − | chr4 | 58556760 | 58556954 | + |
| chr1 | 25547065 | 25547201 | − | chr19 | 15524885 | 15525087 | − | chr4 | 58735725 | 58735800 | − |
| chr1 | 25547529 | 25547626 | − | chr19 | 15528038 | 15528158 | + | chr4 | 58736080 | 58736408 | − |
| chr1 | 26004373 | 26004579 | + | chr19 | 15528373 | 15528681 | + | chr4 | 59309992 | 59310470 | − |
| chr1 | 29343184 | 29343629 | + | chr19 | 15556899 | 15557220 | + | chr4 | 59979196 | 59979530 | + |
| chr1 | 29446510 | 29446648 | − | chr19 | 15557211 | 15557292 | + | chr4 | 60272051 | 60272443 | − |
| chr1 | 29849253 | 29849656 | − | chr19 | 15590386 | 15590728 | + | chr4 | 60399355 | 60399398 | + |
| chr1 | 30244552 | 30244818 | + | chr19 | 15869221 | 15869514 | − | chr4 | 60860625 | 60860755 | − |
| chr1 | 30447131 | 30447357 | − | chr19 | 16050692 | 16050903 | − | chr4 | 60861050 | 60861332 | − |
| chr1 | 30476464 | 30476633 | − | chr19 | 16062626 | 16063062 | + | chr4 | 62853500 | 62853774 | − |
| chr1 | 30476645 | 30476749 | − | chr19 | 16526101 | 16526212 | + | chr4 | 62853895 | 62853952 | − |
| chr1 | 30598765 | 30598837 | − | chr19 | 16792093 | 16792248 | − | chr4 | 63375354 | 63375803 | + |
| chr1 | 31506570 | 31507022 | − | chr19 | 17584492 | 17584609 | − | chr4 | 63623224 | 63623526 | − |
| chr1 | 31849465 | 31849798 | + | chr19 | 17646801 | 17647062 | + | chr4 | 63799025 | 63799489 | − |
| chr1 | 31877693 | 31877942 | + | chr19 | 17838461 | 17838516 | − | chr4 | 63894304 | 63894506 | − |
| chr1 | 33186883 | 33187349 | − | chr19 | 17839017 | 17839205 | − | chr4 | 63976112 | 63976153 | + |
| chr1 | 33192507 | 33192705 | − | chr19 | 18412935 | 18413138 | − | chr4 | 65761745 | 65761856 | − |
| chr1 | 33192719 | 33192763 | − | chr19 | 18984337 | 18984489 | − | chr4 | 66737976 | 66738292 | − |
| chr1 | 33894271 | 33894739 | + | chr19 | 19116020 | 19116303 | + | chr4 | 66914963 | 66915371 | + |
| chr1 | 34056733 | 34057040 | − | chr19 | 22551916 | 22552323 | − | chr4 | 67032851 | 67033193 | − |
| chr1 | 34126092 | 34126251 | + | chr19 | 28158677 | 28158818 | − | chr4 | 67036681 | 67036973 | − |
| chr1 | 34433492 | 34433901 | − | chr19 | 28159109 | 28159152 | − | chr4 | 69623317 | 69623483 | − |
| chr1 | 34484020 | 34484374 | + | chr19 | 28193405 | 28193853 | − | chr4 | 69683822 | 69683988 | + |
| chr1 | 34531907 | 34532351 | − | chr19 | 28231358 | 28231716 | − | chr4 | 71650206 | 71650622 | + |
| chr1 | 34750175 | 34750594 | − | chr19 | 28250818 | 28251200 | − | chr4 | 72143669 | 72143907 | + |
| chr1 | 34826422 | 34826947 | − | chr19 | 28293191 | 28293452 | − | chr4 | 72144477 | 72144534 | + |
| chr1 | 35789257 | 35789614 | − | chr19 | 28303786 | 28304111 | + | chr4 | 72956717 | 72956899 | − |
| chr1 | 36566187 | 36566669 | + | chr19 | 28332874 | 28333125 | + | chr4 | 72956912 | 72957024 | − |
| chr1 | 36639694 | 36639843 | − | chr19 | 28333491 | 28333688 | + | chr4 | 73352445 | 73352636 | + |
| chr1 | 36679620 | 36679872 | + | chr19 | 28374167 | 28374608 | + | chr4 | 73478373 | 73478625 | + |
| chr1 | 36688178 | 36688300 | − | chr19 | 28395288 | 28395343 | − | chr4 | 73932211 | 73932290 | + |
| chr1 | 36689033 | 36689318 | − | chr19 | 28395607 | 28395661 | − | chr4 | 73932265 | 73932429 | + |
| chr1 | 36789511 | 36789946 | + | chr19 | 28395961 | 28395971 | − | chr4 | 74126004 | 74126112 | + |
| chr1 | 36987323 | 36987644 | − | chr19 | 28396261 | 28396356 | − | chr4 | 75160775 | 75161121 | − |
| chr1 | 37001695 | 37001988 | − | chr19 | 28396840 | 28396907 | − | chr4 | 75425297 | 75425566 | + |
| chr1 | 38058980 | 38059437 | − | chr19 | 28396989 | 28397079 | − | chr4 | 75890792 | 75890900 | + |
| chr1 | 38208681 | 38208888 | + | chr19 | 28489196 | 28489402 | − | chr4 | 75891211 | 75891490 | + |
| chr1 | 38209113 | 38209301 | + | chr19 | 28500557 | 28501022 | + | chr4 | 76448367 | 76448453 | + |
| chr1 | 38245010 | 38245343 | − | chr19 | 28574976 | 28575141 | − | chr4 | 76448474 | 76448766 | + |
| chr1 | 38673377 | 38673600 | − | chr19 | 28576567 | 28576788 | − | chr4 | 77140750 | 77140830 | − |
| chr1 | 38755490 | 38755656 | + | chr19 | 28594328 | 28594381 | + | chr4 | 77232197 | 77232381 | − |
| chr1 | 38756222 | 38756320 | + | chr19 | 28866717 | 28867132 | − | chr4 | 77290250 | 77290423 | − |
| chr1 | 40438372 | 40438812 | − | chr19 | 28903983 | 28904228 | + | chr4 | 77420661 | 77420724 | − |
| chr1 | 40965122 | 40965283 | − | chr19 | 28912356 | 28912661 | − | chr4 | 77420791 | 77420972 | − |
| chr1 | 41271018 | 41271483 | − | chr19 | 28994775 | 28994972 | − | chr4 | 77482582 | 77482751 | + |
| chr1 | 41464220 | 41464558 | + | chr19 | 29038835 | 29039190 | + | chr4 | 77490147 | 77490479 | − |
| chr1 | 41464960 | 41465033 | + | chr19 | 29039595 | 29039740 | + | chr4 | 77563482 | 77563530 | + |
| chr1 | 41467665 | 41467964 | − | chr19 | 29130126 | 29130412 | − | chr4 | 77624154 | 77624266 | − |
| chr1 | 42220509 | 42220870 | + | chr19 | 29130934 | 29131110 | − | chr4 | 77642261 | 77642534 | − |
| chr1 | 42392455 | 42392676 | + | chr19 | 29373309 | 29373758 | − | chr4 | 78621460 | 78621836 | + |
| chr1 | 42393013 | 42393251 | + | chr19 | 29429070 | 29429453 | + | chr4 | 79189540 | 79189783 | + |
| chr1 | 42824525 | 42824995 | − | chr19 | 29502096 | 29502342 | − | chr4 | 79455876 | 79456324 | − |
| chr1 | 43018622 | 43019106 | − | chr19 | 29580929 | 29581117 | − | chr4 | 80143189 | 80143252 | + |
| chr1 | 43507201 | 43507673 | − | chr19 | 29581470 | 29581793 | − | chr4 | 80144850 | 80144998 | + |
| chr1 | 44459932 | 44460364 | − | chr19 | 29631605 | 29631801 | − | chr4 | 81276456 | 81276635 | − |
| chr1 | 45262011 | 45262393 | + | chr19 | 29673301 | 29673391 | + | chr4 | 81347722 | 81348018 | − |
| chr1 | 48251474 | 48251922 | + | chr19 | 29673548 | 29673670 | + | chr4 | 82055505 | 82055795 | − |
| chr1 | 48495563 | 48495993 | − | chr19 | 29887485 | 29887770 | + | chr4 | 82144429 | 82144858 | + |
| chr1 | 48512608 | 48513071 | + | chr19 | 30095647 | 30096065 | − | chr4 | 82193035 | 82193374 | − |
| chr1 | 48699417 | 48699876 | + | chr19 | 30123918 | 30124042 | − | chr4 | 82770648 | 82770953 | + |
| chr1 | 48859217 | 48859584 | + | chr19 | 30303816 | 30304214 | + | chr4 | 82771257 | 82771399 | + |
| chr1 | 50285539 | 50285881 | − | chr19 | 30589554 | 30589876 | − | chr4 | 83643657 | 83643831 | − |
| chr1 | 50372782 | 50373229 | + | chr19 | 30672703 | 30672987 | − | chr4 | 84181736 | 84182109 | − |
| chr1 | 50398182 | 50398475 | − | chr19 | 30674516 | 30674638 | − | chr4 | 85514182 | 85514375 | + |
| chr1 | 50411846 | 50411941 | − | chr19 | 30858150 | 30858522 | + | chr4 | 85514855 | 85515090 | − |
| chr1 | 51073303 | 51073650 | − | chr19 | 30996296 | 30996645 | − | chr4 | 86293471 | 86293543 | + |
| chr1 | 51603779 | 51604210 | − | chr19 | 31257928 | 31258325 | − | chr4 | 86293958 | 86294343 | + |
| chr1 | 52638522 | 52638930 | − | chr19 | 31264218 | 31264519 | + | chr4 | 86506533 | 86506736 | − |
| chr1 | 54019196 | 54019503 | − | chr19 | 31399481 | 31399711 | + | chr4 | 87230972 | 87231406 | + |
| chr1 | 54019797 | 54019949 | − | chr19 | 31432351 | 31432519 | + | chr4 | 87493093 | 87493214 | + |
| chr1 | 54116284 | 54116772 | − | chr19 | 31436693 | 31436882 | + | chr4 | 87718179 | 87718588 | − |
| chr1 | 54141728 | 54141979 | − | chr19 | 31437268 | 31437495 | + | chr4 | 87859290 | 87859653 | + |
| chr1 | 54154920 | 54155345 | − | chr19 | 31797114 | 31797550 | − | chr4 | 88077981 | 88078354 | − |
| chr1 | 54167457 | 54167813 | + | chr19 | 32015756 | 32015946 | + | chr4 | 88925967 | 88926269 | − |
| chr1 | 54513350 | 54513530 | − | chr19 | 32017143 | 32017246 | + | chr4 | 90980910 | 90981342 | + |
| chr1 | 54774658 | 54774983 | − | chr19 | 33339186 | 33339641 | − | chr4 | 91314842 | 91315150 | − |
| chr1 | 54845389 | 54845735 | + | chr19 | 33826801 | 33826951 | + | chr4 | 91315181 | 91315334 | − |
| chr1 | 55419008 | 55419192 | − | chr19 | 33827973 | 33828128 | + | chr4 | 91407897 | 91408067 | − |
| chr1 | 55419300 | 55419425 | − | chr19 | 35887015 | 35887404 | − | chr4 | 91845541 | 91845665 | + |
| chr1 | 55744158 | 55744481 | + | chr19 | 36003039 | 36003198 | + | chr4 | 91849095 | 91849281 | + |
| chr1 | 55832186 | 55832621 | + | chr19 | 37505998 | 37506297 | + | chr4 | 91985361 | 91985571 | − |
| chr1 | 55990447 | 55990626 | − | chr19 | 39292671 | 39292803 | − | chr4 | 93299032 | 93299136 | − |
| chr1 | 55990619 | 55990713 | − | chr19 | 39293229 | 39293505 | − | chr4 | 95251373 | 95251424 | + |
| chr1 | 56093459 | 56093913 | + | chr19 | 40683193 | 40683549 | + | chr4 | 95634891 | 95635226 | + |
| chr1 | 56135812 | 56136231 | − | chr19 | 44555707 | 44555757 | − | chr4 | 95798795 | 95799196 | + |
| chr1 | 57207993 | 57208266 | − | chr19 | 44555951 | 44556093 | − | chr4 | 96124685 | 96124999 | + |
| chr1 | 58037049 | 58037358 | + | chr19 | 44556406 | 44556518 | − | chr4 | 96990437 | 96990734 | − |
| chr1 | 58037712 | 58037885 | + | chr19 | 44623129 | 44623523 | − | chr4 | 97228836 | 97228938 | − |
| chr1 | 58204434 | 58204750 | − | chr19 | 47424174 | 47424296 | + | chr4 | 97228938 | 97229206 | − |
| chr1 | 58368671 | 58368828 | + | chr19 | 47424605 | 47424733 | + | chr4 | 97653755 | 97654194 | − |
| chr1 | 58369022 | 58369323 | + | chr19 | 48154346 | 48154662 | + | chr4 | 98695392 | 98695437 | − |
| chr1 | 58382017 | 58382072 | + | chr19 | 57340432 | 57340563 | + | chr4 | 98695735 | 98695814 | − |
| chr1 | 58440812 | 58441251 | − | chr1_KI270708v1_random | 121274 | 121490 | + | chr4 | 98696282 | 98696660 | − |
| chr1 | 58455070 | 58455523 | − | chr1_KI270712v1_random | 37265 | 37355 | − | chr4 | 99200503 | 99200566 | − |
| chr1 | 58556319 | 58556776 | − | chr1_KI270759v1_alt | 128798 | 129243 | + | chr4 | 99200590 | 99200805 | − |
| chr1 | 59960086 | 59960416 | − | chr1_KI270760v1_alt | 100058 | 100284 | − | chr4 | 100874975 | 100875198 | − |
| chr1 | 60098986 | 60099321 | + | chr2 | 3443282 | 3443394 | + | chr4 | 101823928 | 101824369 | − |
| chr1 | 60653875 | 60654288 | − | chr2 | 4231761 | 4232204 | − | chr4 | 103757923 | 103758042 | − |
| chr1 | 60796393 | 60796470 | + | chr2 | 5598083 | 5598458 | + | chr4 | 104293052 | 104293293 | − |
| chr1 | 60796620 | 60796814 | + | chr2 | 6967511 | 6967797 | + | chr4 | 105094351 | 105094645 | + |
| chr1 | 61178774 | 61179097 | − | chr2 | 7195333 | 7195808 | + | chr4 | 105601375 | 105601824 | − |
| chr1 | 63080493 | 63080923 | − | chr2 | 8509836 | 8509952 | + | chr4 | 107742404 | 107742558 | − |
| chr1 | 63688954 | 63689370 | − | chr2 | 8510810 | 8510919 | + | chr4 | 107742853 | 107743176 | − |
| chr1 | 63751116 | 63751552 | + | chr2 | 10954617 | 10954833 | + | chr4 | 108188893 | 108189371 | + |
| chr1 | 64347861 | 64348227 | + | chr2 | 10964504 | 10964751 | + | chr4 | 108468010 | 108468484 | − |
| chr1 | 65091638 | 65091876 | + | chr2 | 12064538 | 12064706 | + | chr4 | 109008187 | 109008529 | + |
| chr1 | 65092710 | 65092921 | + | chr2 | 12065157 | 12065278 | + | chr4 | 109386113 | 109386440 | − |
| chr1 | 65137927 | 65137966 | + | chr2 | 12244067 | 12244371 | − | chr4 | 109393859 | 109394324 | − |
| chr1 | 65138034 | 65138312 | + | chr2 | 12994207 | 12994263 | − | chr4 | 115407936 | 115408308 | − |
| chr1 | 66864448 | 66864540 | − | chr2 | 12994618 | 12995028 | − | chr4 | 115442107 | 115442588 | + |
| chr1 | 67143328 | 67143732 | − | chr2 | 14192914 | 14192997 | + | chr4 | 116024938 | 116025404 | + |
| chr1 | 67835795 | 67836088 | + | chr2 | 14193031 | 14193159 | + | chr4 | 116212508 | 116212752 | − |
| chr1 | 67935539 | 67935596 | − | chr2 | 14297997 | 14298143 | − | chr4 | 116217746 | 116218013 | − |
| chr1 | 67936070 | 67936485 | − | chr2 | 14869246 | 14869492 | + | chr4 | 116389303 | 116389598 | − |
| chr1 | 68119626 | 68119775 | + | chr2 | 15097037 | 15097220 | − | chr4 | 117322982 | 117323111 | + |
| chr1 | 69122639 | 69123098 | − | chr2 | 15106700 | 15107000 | − | chr4 | 117900325 | 117900593 | + |
| chr1 | 69707141 | 69707580 | + | chr2 | 15123349 | 15123390 | − | chr4 | 117901064 | 117901168 | + |
| chr1 | 69832043 | 69832462 | − | chr2 | 15126407 | 15126767 | − | chr4 | 117930450 | 117930642 | − |
| chr1 | 70821317 | 70821470 | − | chr2 | 15389195 | 15389369 | + | chr4 | 117957695 | 117958180 | + |
| chr1 | 70821514 | 70821638 | − | chr2 | 20914621 | 20914786 | + | chr4 | 119475049 | 119475463 | + |
| chr1 | 70908358 | 70908673 | + | chr2 | 20914800 | 20914884 | + | chr4 | 120087880 | 120087993 | + |
| chr1 | 71202711 | 71203153 | − | chr2 | 21720706 | 21721190 | − | chr4 | 120526074 | 120526154 | − |
| chr1 | 72845982 | 72846345 | + | chr2 | 22302102 | 22302412 | − | chr4 | 121524010 | 121524417 | − |
| chr1 | 72966692 | 72967149 | − | chr2 | 23464295 | 23464612 | + | chr4 | 122163117 | 122163571 | − |
| chr1 | 73170231 | 73170647 | − | chr2 | 25655082 | 25655126 | + | chr4 | 123582278 | 123582665 | − |
| chr1 | 73173934 | 73174083 | − | chr2 | 25655779 | 25656161 | + | chr4 | 124019897 | 124020154 | + |
| chr1 | 73754287 | 73754608 | − | chr2 | 25899474 | 25899692 | + | chr4 | 125134807 | 125135078 | + |
| chr1 | 73872091 | 73872263 | + | chr2 | 25900594 | 25900644 | + | chr4 | 125828211 | 125828344 | − |
| chr1 | 73890837 | 73891014 | − | chr2 | 25900934 | 25901055 | + | chr4 | 125889642 | 125889875 | + |
| chr1 | 75569799 | 75570269 | − | chr2 | 27967100 | 27967169 | − | chr4 | 125890794 | 125890924 | + |
| chr1 | 75864806 | 75865237 | + | chr2 | 27969319 | 27969634 | − | chr4 | 125940063 | 125940271 | + |
| chr1 | 76151441 | 76151674 | − | chr2 | 29939794 | 29940242 | − | chr4 | 126610838 | 126611216 | + |
| chr1 | 76151667 | 76151794 | − | chr2 | 29987849 | 29988208 | + | chr4 | 126727802 | 126728095 | + |
| chr1 | 76784109 | 76784295 | − | chr2 | 30087030 | 30087261 | + | chr4 | 126816755 | 126817076 | − |
| chr1 | 76784343 | 76784461 | − | chr2 | 30087417 | 30087611 | + | chr4 | 127210395 | 127210489 | − |
| chr1 | 76858199 | 76858653 | + | chr2 | 30377282 | 30377755 | + | chr4 | 127561936 | 127562240 | + |
| chr1 | 79140771 | 79141192 | − | chr2 | 34736271 | 34736329 | − | chr4 | 128356467 | 128356901 | + |
| chr1 | 79468139 | 79468421 | + | chr2 | 37332047 | 37332174 | − | chr4 | 128992377 | 128992849 | + |
| chr1 | 79570114 | 79570570 | − | chr2 | 41182796 | 41183241 | − | chr4 | 129391012 | 129391369 | + |
| chr1 | 80173056 | 80173120 | − | chr2 | 41572449 | 41572707 | + | chr4 | 129391951 | 129391969 | + |
| chr1 | 80173116 | 80173221 | − | chr2 | 46779900 | 46780291 | + | chr4 | 129393751 | 129393872 | + |
| chr1 | 80973705 | 80973928 | + | chr2 | 49748589 | 49748709 | − | chr4 | 129747620 | 129748066 | − |
| chr1 | 81006485 | 81006797 | + | chr2 | 49914415 | 49914839 | + | chr4 | 131311913 | 131312183 | + |
| chr1 | 81597808 | 81598049 | − | chr2 | 51082133 | 51082576 | + | chr4 | 134147772 | 134148186 | − |
| chr1 | 81634951 | 81635331 | + | chr2 | 52157049 | 52157119 | + | chr4 | 134412857 | 134413251 | − |
| chr1 | 82570834 | 82571043 | − | chr2 | 53096533 | 53096587 | + | chr4 | 134447170 | 134447358 | − |
| chr1 | 82806025 | 82806310 | − | chr2 | 53096603 | 53096751 | + | chr4 | 134893615 | 134894038 | − |
| chr1 | 83066401 | 83066790 | + | chr2 | 56151656 | 56152067 | − | chr4 | 135065724 | 135066023 | − |
| chr1 | 83068727 | 83068797 | + | chr2 | 57183167 | 57183243 | + | chr4 | 135278143 | 135278352 | + |
| chr1 | 83206253 | 83206528 | + | chr2 | 57563402 | 57563784 | − | chr4 | 135278411 | 135278526 | + |
| chr1 | 83206543 | 83206660 | + | chr2 | 58434548 | 58434998 | − | chr4 | 135552528 | 135552800 | + |
| chr1 | 83218095 | 83218416 | − | chr2 | 59548056 | 59548431 | − | chr4 | 136095370 | 136095656 | − |
| chr1 | 83306396 | 83306687 | + | chr2 | 59717345 | 59717537 | + | chr4 | 137025574 | 137025959 | − |
| chr1 | 83486416 | 83486766 | − | chr2 | 61808010 | 61808116 | − | chr4 | 140187388 | 140187818 | − |
| chr1 | 83970691 | 83971179 | − | chr2 | 62227785 | 62228015 | − | chr4 | 140557242 | 140557373 | − |
| chr1 | 85188503 | 85188763 | − | chr2 | 64319480 | 64319625 | − | chr4 | 140558539 | 140558801 | − |
| chr1 | 85188791 | 85188906 | − | chr2 | 64319924 | 64320214 | − | chr4 | 140984590 | 140985046 | + |
| chr1 | 85292897 | 85293109 | − | chr2 | 64380054 | 64380468 | − | chr4 | 141369692 | 141370096 | + |
| chr1 | 86795756 | 86796207 | − | chr2 | 64409848 | 64410326 | − | chr4 | 141560017 | 141560466 | − |
| chr1 | 87475151 | 87475444 | + | chr2 | 65488762 | 65489077 | − | chr4 | 141576768 | 141577146 | + |
| chr1 | 89275321 | 89275638 | − | chr2 | 65524643 | 65525088 | − | chr4 | 142553486 | 142553822 | + |
| chr1 | 90100541 | 90101030 | + | chr2 | 67177688 | 67177883 | − | chr4 | 143844641 | 143845073 | + |
| chr1 | 93716516 | 93716908 | − | chr2 | 67186835 | 67187129 | + | chr4 | 143845387 | 143845427 | + |
| chr1 | 94622672 | 94622918 | − | chr2 | 68006944 | 68007344 | − | chr4 | 143965632 | 143966068 | + |
| chr1 | 94630284 | 94630661 | + | chr2 | 70771784 | 70772105 | + | chr4 | 144068841 | 144069274 | + |
| chr1 | 94705789 | 94706239 | + | chr2 | 71168992 | 71169010 | + | chr4 | 144069587 | 144069627 | + |
| chr1 | 95534976 | 95535294 | − | chr2 | 71169571 | 71169824 | + | chr4 | 144341607 | 144342039 | − |
| chr1 | 95665312 | 95665469 | + | chr2 | 71253447 | 71253911 | − | chr4 | 144818495 | 144818934 | + |
| chr1 | 95666426 | 95666545 | + | chr2 | 71741193 | 71741512 | − | chr4 | 145814280 | 145814606 | + |
| chr1 | 96352050 | 96352437 | + | chr2 | 71842278 | 71842706 | + | chr4 | 145814964 | 145815020 | + |
| chr1 | 96414488 | 96414912 | − | chr2 | 71927300 | 71927703 | − | chr4 | 146600318 | 146600677 | − |
| chr1 | 96989091 | 96989379 | − | chr2 | 72688228 | 72688529 | − | chr4 | 146853884 | 146854252 | + |
| chr1 | 97629229 | 97629355 | − | chr2 | 72688563 | 72688717 | − | chr4 | 147594244 | 147594632 | + |
| chr1 | 97629379 | 97629498 | − | chr2 | 75020516 | 75020609 | + | chr4 | 149306310 | 149306609 | + |
| chr1 | 98556546 | 98556834 | − | chr2 | 75020621 | 75020932 | + | chr4 | 149312676 | 149313049 | + |
| chr1 | 98574808 | 98574920 | − | chr2 | 75119839 | 75120064 | − | chr4 | 149549996 | 149550409 | + |
| chr1 | 99360529 | 99360986 | − | chr2 | 75120151 | 75120284 | − | chr4 | 150530501 | 150530744 | + |
| chr1 | 99432208 | 99432455 | − | chr2 | 78880347 | 78880561 | + | chr4 | 152554655 | 152554948 | − |
| chr1 | 100335472 | 100335808 | + | chr2 | 79266096 | 79266544 | + | chr4 | 154890814 | 154891148 | + |
| chr1 | 100950786 | 100951243 | − | chr2 | 79830118 | 79830384 | − | chr4 | 155932652 | 155932821 | + |
| chr1 | 102232267 | 102232522 | − | chr2 | 80890191 | 80890482 | − | chr4 | 155974882 | 155975348 | − |
| chr1 | 103296466 | 103296827 | − | chr2 | 82974022 | 82974307 | + | chr4 | 156924544 | 156924995 | − |
| chr1 | 104149268 | 104149700 | + | chr2 | 83534153 | 83534469 | − | chr4 | 156991739 | 156992065 | + |
| chr1 | 106202142 | 106202554 | − | chr2 | 84042582 | 84042912 | + | chr4 | 159545501 | 159545686 | − |
| chr1 | 106248339 | 106248512 | − | chr2 | 84061747 | 84061790 | − | chr4 | 159885964 | 159886035 | − |
| chr1 | 106524246 | 106524490 | + | chr2 | 84062530 | 84062710 | − | chr4 | 160511739 | 160511865 | + |
| chr1 | 106525035 | 106525061 | + | chr2 | 84292974 | 84293262 | − | chr4 | 160989711 | 160989922 | + |
| chr1 | 107018924 | 107019363 | − | chr2 | 84293322 | 84293420 | − | chr4 | 160990371 | 160990483 | + |
| chr1 | 108126517 | 108126686 | − | chr2 | 84309265 | 84309585 | − | chr4 | 162262861 | 162263040 | − |
| chr1 | 108126716 | 108126853 | − | chr2 | 85947283 | 85947747 | + | chr4 | 167671130 | 167671273 | + |
| chr1 | 108328754 | 108329153 | + | chr2 | 85987217 | 85987460 | − | chr4 | 167671542 | 167671652 | + |
| chr1 | 110111287 | 110111466 | + | chr2 | 88323925 | 88324029 | − | chr4 | 167694437 | 167694743 | + |
| chr1 | 110111774 | 110112007 | + | chr2 | 88324339 | 88324547 | − | chr4 | 167764883 | 167765270 | − |
| chr1 | 110728541 | 110728564 | + | chr2 | 89042747 | 89042952 | + | chr4 | 168473764 | 168473834 | + |
| chr1 | 110729554 | 110729610 | + | chr2 | 89048399 | 89048454 | + | chr4 | 170998050 | 170998167 | − |
| chr1 | 110730432 | 110730807 | + | chr2 | 89048845 | 89048954 | + | chr4 | 171985018 | 171985125 | + |
| chr1 | 112139990 | 112140188 | + | chr2 | 89103062 | 89103144 | + | chr4 | 172000333 | 172000471 | − |
| chr1 | 112239178 | 112239382 | + | chr2 | 89270312 | 89270374 | + | chr4 | 172050131 | 172050160 | − |
| chr1 | 112239390 | 112239632 | + | chr2 | 89279670 | 89279958 | − | chr4 | 172050837 | 172051049 | − |
| chr1 | 112661226 | 112661557 | + | chr2 | 89288904 | 89289020 | + | chr4 | 172345681 | 172346137 | − |
| chr1 | 112772818 | 112773165 | + | chr2 | 89301450 | 89301652 | + | chr4 | 172533937 | 172534398 | − |
| chr1 | 112790053 | 112790392 | − | chr2 | 89321776 | 89322063 | + | chr4 | 173026108 | 173026543 | − |
| chr1 | 116347794 | 116347952 | + | chr2 | 89860543 | 89860830 | − | chr4 | 173343898 | 173344259 | − |
| chr1 | 116852745 | 116852962 | − | chr2 | 89880827 | 89881029 | − | chr4 | 173653827 | 173654216 | + |
| chr1 | 118671714 | 118672193 | − | chr2 | 89893464 | 89893580 | − | chr4 | 175728716 | 175729192 | + |
| chr1 | 119782750 | 119783025 | + | chr2 | 89902588 | 89902876 | + | chr4 | 176530236 | 176530426 | + |
| chr1 | 120124098 | 120124500 | + | chr2 | 89912172 | 89912234 | − | chr4 | 176755200 | 176755635 | + |
| chr1 | 121414945 | 121415411 | + | chr2 | 90080019 | 90080097 | − | chr4 | 177884576 | 177884821 | − |
| chr1 | 144083861 | 144083951 | − | chr2 | 90097470 | 90097552 | − | chr4 | 178127635 | 178127927 | − |
| chr1 | 144876757 | 144876847 | + | chr2 | 90151637 | 90151719 | − | chr4 | 178389094 | 178389590 | − |
| chr1 | 146285075 | 146285400 | + | chr2 | 90152110 | 90152165 | − | chr4 | 179276591 | 179276887 | − |
| chr1 | 146285712 | 146285805 | + | chr2 | 90157606 | 90157811 | − | chr4 | 179276955 | 179276997 | − |
| chr1 | 146746084 | 146746292 | − | chr2 | 90207262 | 90207344 | − | chr4 | 180939735 | 180939984 | − |
| chr1 | 146909616 | 146909895 | + | chr2 | 90317513 | 90317765 | + | chr4 | 181114512 | 181114952 | − |
| chr1 | 148187134 | 148187413 | − | chr2 | 91484860 | 91485112 | − | chr4 | 181406759 | 181406970 | + |
| chr1 | 148735858 | 148736183 | + | chr2 | 91815846 | 91816098 | − | chr4 | 181829779 | 181829996 | + |
| chr1 | 148736495 | 148736588 | + | chr2 | 92036672 | 92036924 | + | chr4 | 182939827 | 182939998 | + |
| chr1 | 153343638 | 153343704 | − | chr2 | 94267211 | 94267463 | + | chr4 | 183748500 | 183748560 | + |
| chr1 | 153344273 | 153344466 | − | chr2 | 94758204 | 94758284 | − | chr4 | 183749020 | 183749365 | + |
| chr1 | 153344507 | 153344638 | − | chr2 | 94758276 | 94758641 | − | chr4 | 185078060 | 185078122 | + |
| chr1 | 156632230 | 156632587 | − | chr2 | 97058147 | 97058411 | − | chr4 | 185994263 | 185994423 | + |
| chr1 | 156635222 | 156635387 | − | chr2 | 97324060 | 97324405 | + | chr4 | 188395883 | 188396278 | − |
| chr1 | 156635683 | 156635774 | − | chr2 | 97356063 | 97356401 | + | chr4_GL383527v1_alt | 97275 | 97444 | + |
| chr1 | 159992222 | 159992688 | + | chr2 | 97384100 | 97384445 | − | chr4_GL383527v1_alt | 152154 | 152620 | − |
| chr1 | 164184098 | 164184554 | − | chr2 | 99240124 | 99240586 | + | chr4_KI270789v1_alt | 54446 | 54856 | − |
| chr1 | 165009360 | 165009759 | + | chr2 | 100160839 | 100161307 | − | chr5 | 2045445 | 2045818 | + |
| chr1 | 165605418 | 165605551 | − | chr2 | 100759560 | 100759632 | + | chr5 | 3145545 | 3145792 | − |
| chr1 | 165796037 | 165796327 | + | chr2 | 101692008 | 101692170 | + | chr5 | 3375154 | 3375609 | + |
| chr1 | 166009964 | 166010419 | + | chr2 | 103462887 | 103463037 | + | chr5 | 4034553 | 4034676 | − |
| chr1 | 166764404 | 166764859 | + | chr2 | 103463049 | 103463155 | + | chr5 | 4070919 | 4071189 | + |
| chr1 | 166891214 | 166891645 | + | chr2 | 104525182 | 104525262 | − | chr5 | 5093679 | 5094096 | − |
| chr1 | 167502470 | 167502565 | − | chr2 | 104600019 | 104600294 | − | chr5 | 5890658 | 5890876 | + |
| chr1 | 167503020 | 167503159 | − | chr2 | 105636279 | 105636543 | + | chr5 | 6005097 | 6005280 | + |
| chr1 | 169741538 | 169741926 | − | chr2 | 105677477 | 105677601 | − | chr5 | 9736318 | 9736402 | + |
| chr1 | 170328508 | 170328968 | − | chr2 | 105677957 | 105678258 | − | chr5 | 9736446 | 9736712 | + |
| chr1 | 170725336 | 170725770 | + | chr2 | 105679395 | 105679771 | + | chr5 | 10114197 | 10114427 | + |
| chr1 | 172779786 | 172780019 | + | chr2 | 105731011 | 105731432 | − | chr5 | 12153028 | 12153222 | + |
| chr1 | 172892662 | 172893025 | + | chr2 | 105732626 | 105732687 | − | chr5 | 12688349 | 12688607 | + |
| chr1 | 173436121 | 173436406 | − | chr2 | 105733067 | 105733472 | − | chr5 | 12712659 | 12712905 | − |
| chr1 | 175304894 | 175305086 | − | chr2 | 105953750 | 105954169 | − | chr5 | 12783754 | 12784035 | − |
| chr1 | 177228582 | 177228707 | − | chr2 | 106342278 | 106342465 | − | chr5 | 13188777 | 13188922 | + |
| chr1 | 181673014 | 181673186 | − | chr2 | 106905703 | 106905948 | − | chr5 | 13215530 | 13215925 | − |
| chr1 | 181673327 | 181673478 | − | chr2 | 107381882 | 107382015 | − | chr5 | 13648278 | 13648491 | − |
| chr1 | 185912541 | 185912690 | + | chr2 | 107382032 | 107382223 | − | chr5 | 14082483 | 14082778 | − |
| chr1 | 186978713 | 186979180 | − | chr2 | 109122213 | 109122424 | − | chr5 | 14985087 | 14985421 | − |
| chr1 | 187297347 | 187297775 | + | chr2 | 112047473 | 112047825 | + | chr5 | 15134170 | 15134335 | + |
| chr1 | 188092569 | 188092768 | + | chr2 | 112048212 | 112048303 | + | chr5 | 15134541 | 15134739 | + |
| chr1 | 188093828 | 188093897 | + | chr2 | 112480821 | 112481196 | − | chr5 | 15160870 | 15160983 | − |
| chr1 | 188221718 | 188222158 | − | chr2 | 112590455 | 112590817 | − | chr5 | 15203205 | 15203645 | − |
| chr1 | 188228813 | 188229192 | − | chr2 | 112590855 | 112590898 | − | chr5 | 15486383 | 15486840 | − |
| chr1 | 188687834 | 188688019 | − | chr2 | 112593939 | 112594035 | − | chr5 | 17153526 | 17153865 | − |
| chr1 | 189966942 | 189967297 | + | chr2 | 113412960 | 113413240 | + | chr5 | 17154181 | 17154291 | − |
| chr1 | 191868755 | 191869046 | + | chr2 | 115613718 | 115614149 | − | chr5 | 18134263 | 18134463 | − |
| chr1 | 192227646 | 192227894 | + | chr2 | 115934286 | 115934697 | + | chr5 | 18905734 | 18905916 | − |
| chr1 | 192228206 | 192228303 | + | chr2 | 117087311 | 117087482 | − | chr5 | 19067937 | 19068104 | + |
| chr1 | 192837761 | 192838046 | + | chr2 | 119177292 | 119177536 | + | chr5 | 19101840 | 19102140 | − |
| chr1 | 192871487 | 192871556 | − | chr2 | 119490737 | 119491064 | + | chr5 | 19357360 | 19357567 | − |
| chr1 | 192871945 | 192872291 | − | chr2 | 120713470 | 120713535 | − | chr5 | 19357962 | 19358153 | − |
| chr1 | 192908188 | 192908643 | − | chr2 | 120713994 | 120714328 | − | chr5 | 21160450 | 21160765 | + |
| chr1 | 193451038 | 193451276 | − | chr2 | 120813871 | 120814287 | − | chr5 | 21633753 | 21633976 | − |
| chr1 | 193451683 | 193451818 | − | chr2 | 120892577 | 120892930 | + | chr5 | 21714811 | 21715074 | + |
| chr1 | 193625189 | 193625489 | − | chr2 | 121995881 | 121996142 | − | chr5 | 23274520 | 23274960 | − |
| chr1 | 194503147 | 194503360 | + | chr2 | 122098880 | 122099244 | + | chr5 | 23650074 | 23650422 | + |
| chr1 | 194503735 | 194503916 | + | chr2 | 122729457 | 122729653 | + | chr5 | 25244368 | 25244676 | + |
| chr1 | 196605202 | 196605444 | − | chr2 | 122814945 | 122815078 | − | chr5 | 25406089 | 25406311 | + |
| chr1 | 199728185 | 199728351 | + | chr2 | 122815683 | 122815966 | − | chr5 | 25761417 | 25761543 | − |
| chr1 | 201727580 | 201727920 | − | chr2 | 122895219 | 122895348 | + | chr5 | 25761591 | 25761695 | − |
| chr1 | 205474764 | 205474892 | + | chr2 | 122897763 | 122898196 | + | chr5 | 27057851 | 27058223 | − |
| chr1 | 205475267 | 205475333 | + | chr2 | 124154255 | 124154385 | − | chr5 | 28589416 | 28589507 | − |
| chr1 | 205475517 | 205475775 | + | chr2 | 124235460 | 124235714 | + | chr5 | 29501018 | 29501385 | + |
| chr1 | 205476435 | 205476477 | + | chr2 | 124890138 | 124890580 | + | chr5 | 30551156 | 30551538 | − |
| chr1 | 207146612 | 207147056 | − | chr2 | 125167774 | 125167905 | + | chr5 | 30569563 | 30570085 | − |
| chr1 | 207154723 | 207155161 | − | chr2 | 125542555 | 125542688 | − | chr5 | 31670721 | 31671034 | − |
| chr1 | 207187620 | 207187826 | + | chr2 | 125542726 | 125542837 | − | chr5 | 32221220 | 32221518 | + |
| chr1 | 207203014 | 207203289 | − | chr2 | 126858331 | 126858743 | + | chr5 | 32494958 | 32495430 | − |
| chr1 | 207203618 | 207203813 | − | chr2 | 128250731 | 128250914 | − | chr5 | 32654435 | 32654707 | − |
| chr1 | 207437491 | 207437833 | − | chr2 | 128527989 | 128528192 | + | chr5 | 33623317 | 33623449 | + |
| chr1 | 208864631 | 208865053 | + | chr2 | 128528424 | 128528546 | + | chr5 | 33655230 | 33655602 | + |
| chr1 | 212244509 | 212244911 | + | chr2 | 128997716 | 128997789 | − | chr5 | 33922562 | 33923055 | − |
| chr1 | 213484716 | 213484859 | − | chr2 | 128997855 | 128997903 | − | chr5 | 34449664 | 34449935 | − |
| chr1 | 213485166 | 213485380 | − | chr2 | 128998409 | 128998438 | − | chr5 | 34591482 | 34591922 | − |
| chr1 | 214884673 | 214884917 | − | chr2 | 130047409 | 130047489 | − | chr5 | 34758296 | 34758352 | − |
| chr1 | 216987675 | 216988120 | + | chr2 | 130047480 | 130047787 | − | chr5 | 34758900 | 34758978 | − |
| chr1 | 218929542 | 218929846 | − | chr2 | 130374805 | 130375044 | + | chr5 | 35244803 | 35244870 | − |
| chr1 | 219788027 | 219788314 | − | chr2 | 130438214 | 130438294 | − | chr5 | 35244870 | 35245127 | − |
| chr1 | 220474776 | 220474850 | + | chr2 | 130438285 | 130438592 | − | chr5 | 35361668 | 35362044 | − |
| chr1 | 220476260 | 220476470 | + | chr2 | 130682923 | 130683230 | + | chr5 | 35404941 | 35405291 | + |
| chr1 | 221137568 | 221138035 | + | chr2 | 130683221 | 130683301 | + | chr5 | 35425200 | 35425406 | − |
| chr1 | 221450152 | 221450354 | − | chr2 | 130748938 | 130749289 | + | chr5 | 36159446 | 36159847 | + |
| chr1 | 221852151 | 221852510 | − | chr2 | 131289621 | 131289928 | + | chr5 | 36444421 | 36444825 | + |
| chr1 | 221852825 | 221852928 | − | chr2 | 131289919 | 131289999 | + | chr5 | 36458878 | 36459322 | − |
| chr1 | 222293099 | 222293533 | + | chr2 | 132025386 | 132025519 | + | chr5 | 36530825 | 36531106 | − |
| chr1 | 224205911 | 224206071 | − | chr2 | 132753903 | 132754036 | + | chr5 | 36531183 | 36531229 | − |
| chr1 | 224208287 | 224208352 | − | chr2 | 132754175 | 132754319 | + | chr5 | 36725316 | 36725388 | − |
| chr1 | 224828493 | 224828916 | − | chr2 | 132936845 | 132936903 | − | chr5 | 36748843 | 36749148 | + |
| chr1 | 225282455 | 225282742 | − | chr2 | 132936966 | 132937298 | − | chr5 | 36786959 | 36787230 | − |
| chr1 | 226033708 | 226034079 | − | chr2 | 132969418 | 132969501 | − | chr5 | 37860703 | 37861005 | − |
| chr1 | 229885737 | 229886129 | + | chr2 | 132969500 | 132969685 | − | chr5 | 37901744 | 37902080 | − |
| chr1 | 229886539 | 229886612 | + | chr2 | 134844349 | 134844776 | − | chr5 | 37948843 | 37949061 | − |
| chr1 | 232058405 | 232058659 | − | chr2 | 136494403 | 136494522 | − | chr5 | 37949292 | 37949419 | − |
| chr1 | 232747100 | 232747470 | + | chr2 | 136691361 | 136691561 | − | chr5 | 37965254 | 37965536 | − |
| chr1 | 232952858 | 232953272 | + | chr2 | 136691620 | 136691771 | − | chr5 | 37976780 | 37976909 | − |
| chr1 | 233757321 | 233757791 | − | chr2 | 136992413 | 136992733 | − | chr5 | 37977263 | 37977459 | − |
| chr1 | 238395199 | 238395361 | − | chr2 | 137176572 | 137176973 | + | chr5 | 38282221 | 38282609 | − |
| chr1 | 238395657 | 238395732 | − | chr2 | 137464292 | 137464691 | − | chr5 | 38290065 | 38290125 | − |
| chr1 | 238511989 | 238512279 | + | chr2 | 137797157 | 137797604 | + | chr5 | 38290165 | 38290344 | − |
| chr1 | 238512318 | 238512392 | + | chr2 | 138178753 | 138179128 | − | chr5 | 38748622 | 38748998 | − |
| chr1 | 238712134 | 238712461 | − | chr2 | 138486407 | 138486699 | + | chr5 | 39115837 | 39116211 | + |
| chr1 | 240587885 | 240588386 | + | chr2 | 138627366 | 138627672 | + | chr5 | 40287430 | 40287853 | + |
| chr1 | 244087762 | 244088181 | + | chr2 | 139884835 | 139885212 | + | chr5 | 40399638 | 40399694 | − |
| chr1 | 245108929 | 245109205 | − | chr2 | 140400122 | 140400563 | + | chr5 | 40400060 | 40400167 | − |
| chr1 | 245434494 | 245434939 | + | chr2 | 141089600 | 141089900 | − | chr5 | 40400283 | 40400597 | − |
| chr1 | 246425556 | 246425790 | + | chr2 | 141252273 | 141252317 | + | chr5 | 40907143 | 40907383 | + |
| chr1 | 246978487 | 246978574 | − | chr2 | 143447893 | 143448078 | − | chr5 | 40907709 | 40907959 | + |
| chr1 | 248247461 | 248247714 | − | chr2 | 145159805 | 145160250 | − | chr5 | 40983214 | 40983272 | − |
| chr10 | 778314 | 778392 | + | chr2 | 146027192 | 146027413 | − | chr5 | 41004027 | 41004274 | − |
| chr10 | 778397 | 778466 | + | chr2 | 146416770 | 146416897 | − | chr5 | 41243631 | 41244090 | + |
| chr10 | 778471 | 778544 | + | chr2 | 146622632 | 146623007 | + | chr5 | 41449501 | 41449770 | − |
| chr10 | 1160900 | 1161033 | + | chr2 | 147289556 | 147290025 | + | chr5 | 42311446 | 42311630 | + |
| chr10 | 1946490 | 1946820 | + | chr2 | 147383033 | 147383230 | + | chr5 | 43256553 | 43256983 | + |
| chr10 | 2094290 | 2094433 | − | chr2 | 148161968 | 148162059 | + | chr5 | 44192816 | 44192853 | − |
| chr10 | 2869270 | 2869437 | − | chr2 | 148162092 | 148162388 | + | chr5 | 44735727 | 44735970 | − |
| chr10 | 2869539 | 2869711 | − | chr2 | 148915680 | 148916099 | − | chr5 | 45075806 | 45076215 | + |
| chr10 | 3020658 | 3020902 | + | chr2 | 148916100 | 148916201 | − | chr5 | 45076998 | 45077095 | − |
| chr10 | 7075833 | 7076263 | + | chr2 | 149289146 | 149289258 | + | chr5 | 50548389 | 50548533 | + |
| chr10 | 7458412 | 7458752 | + | chr2 | 149622851 | 149623320 | + | chr5 | 50548627 | 50548727 | + |
| chr10 | 9502327 | 9502657 | + | chr2 | 149749044 | 149749251 | + | chr5 | 50553492 | 50553956 | − |
| chr10 | 9503292 | 9503435 | + | chr2 | 151756482 | 151756666 | + | chr5 | 51813723 | 51813872 | − |
| chr10 | 9807022 | 9807313 | − | chr2 | 153046734 | 153046815 | + | chr5 | 51813904 | 51813965 | − |
| chr10 | 10092607 | 10092902 | − | chr2 | 153119670 | 153120025 | + | chr5 | 52481170 | 52481592 | + |
| chr10 | 10938662 | 10938706 | − | chr2 | 153825510 | 153825608 | + | chr5 | 52594621 | 52594705 | − |
| chr10 | 14304706 | 14305097 | + | chr2 | 153825654 | 153825879 | + | chr5 | 52594999 | 52595084 | − |
| chr10 | 14650961 | 14651138 | + | chr2 | 153826473 | 153826612 | + | chr5 | 52857795 | 52858013 | + |
| chr10 | 14655614 | 14655803 | − | chr2 | 154424491 | 154424876 | + | chr5 | 54706759 | 54707201 | + |
| chr10 | 14727372 | 14727750 | + | chr2 | 154622861 | 154623314 | + | chr5 | 54885776 | 54886083 | + |
| chr10 | 14973257 | 14973486 | + | chr2 | 154649106 | 154649578 | + | chr5 | 54929239 | 54929374 | − |
| chr10 | 15027150 | 15027409 | + | chr2 | 155952855 | 155953311 | − | chr5 | 54929672 | 54930015 | − |
| chr10 | 15618984 | 15619276 | + | chr2 | 156092443 | 156092924 | − | chr5 | 55042202 | 55042546 | − |
| chr10 | 17279736 | 17279854 | − | chr2 | 158475780 | 158475879 | + | chr5 | 55059363 | 55059821 | − |
| chr10 | 17425232 | 17425286 | − | chr2 | 160057884 | 160058184 | + | chr5 | 56234172 | 56234446 | + |
| chr10 | 17425771 | 17425838 | − | chr2 | 160536257 | 160536686 | − | chr5 | 56337229 | 56337491 | + |
| chr10 | 17426345 | 17426685 | − | chr2 | 160740908 | 160740983 | + | chr5 | 56422809 | 56423254 | + |
| chr10 | 17816253 | 17816714 | − | chr2 | 160749743 | 160750110 | + | chr5 | 56466188 | 56466639 | − |
| chr10 | 18835339 | 18835352 | + | chr2 | 160750268 | 160750364 | − | chr5 | 56606167 | 56606493 | − |
| chr10 | 18835712 | 18835927 | + | chr2 | 163288101 | 163288156 | − | chr5 | 56606803 | 56606944 | − |
| chr10 | 18835971 | 18836102 | + | chr2 | 163288465 | 163288731 | − | chr5 | 56705045 | 56705498 | + |
| chr10 | 18954549 | 18954685 | + | chr2 | 163809715 | 163810150 | + | chr5 | 56805524 | 56805766 | − |
| chr10 | 18954754 | 18954897 | + | chr2 | 164030652 | 164030895 | − | chr5 | 56805968 | 56806161 | − |
| chr10 | 20064937 | 20065132 | + | chr2 | 164030956 | 164031076 | − | chr5 | 56963566 | 56963911 | − |
| chr10 | 20314413 | 20314686 | − | chr2 | 166762293 | 166762535 | − | chr5 | 57427668 | 57427951 | − |
| chr10 | 20563423 | 20563587 | + | chr2 | 166861272 | 166861510 | − | chr5 | 57746621 | 57747010 | + |
| chr10 | 21204003 | 21204250 | − | chr2 | 167316584 | 167316885 | + | chr5 | 57798331 | 57798444 | + |
| chr10 | 21204668 | 21204860 | − | chr2 | 170151148 | 170151228 | + | chr5 | 57798461 | 57798685 | + |
| chr10 | 21265515 | 21265700 | − | chr2 | 170880171 | 170880393 | + | chr5 | 57948669 | 57949094 | + |
| chr10 | 21265914 | 21266048 | − | chr2 | 172810879 | 172811198 | − | chr5 | 57995414 | 57995841 | + |
| chr10 | 21275356 | 21275614 | + | chr2 | 172884926 | 172885394 | + | chr5 | 58213134 | 58213602 | + |
| chr10 | 22173805 | 22174159 | − | chr2 | 173787871 | 173788300 | + | chr5 | 58816152 | 58816598 | − |
| chr10 | 25368042 | 25368281 | + | chr2 | 174140762 | 174140845 | + | chr5 | 59299159 | 59299532 | + |
| chr10 | 29773388 | 29773821 | − | chr2 | 174767015 | 174767312 | + | chr5 | 59369395 | 59369704 | − |
| chr10 | 31195234 | 31195588 | + | chr2 | 180595878 | 180595965 | − | chr5 | 60220009 | 60220294 | − |
| chr10 | 31210790 | 31211013 | + | chr2 | 180596339 | 180596389 | − | chr5 | 60833412 | 60833568 | + |
| chr10 | 31644023 | 31644067 | − | chr2 | 180597246 | 180597386 | − | chr5 | 61136580 | 61136902 | − |
| chr10 | 31644430 | 31644630 | − | chr2 | 180612597 | 180612787 | − | chr5 | 61602003 | 61602482 | + |
| chr10 | 32178921 | 32178987 | − | chr2 | 180781399 | 180781714 | − | chr5 | 61971375 | 61971710 | + |
| chr10 | 32179550 | 32179719 | − | chr2 | 181701540 | 181701947 | − | chr5 | 61974074 | 61974218 | − |
| chr10 | 34307221 | 34307417 | − | chr2 | 181717488 | 181717943 | − | chr5 | 62938264 | 62938691 | − |
| chr10 | 36497860 | 36498214 | − | chr2 | 182297614 | 182297759 | + | chr5 | 63248924 | 63249301 | + |
| chr10 | 36498512 | 36498569 | − | chr2 | 182297786 | 182297940 | + | chr5 | 63580967 | 63581053 | − |
| chr10 | 37450790 | 37451140 | + | chr2 | 182302134 | 182302304 | + | chr5 | 63581094 | 63581378 | − |
| chr10 | 42183381 | 42183650 | − | chr2 | 182304962 | 182305252 | + | chr5 | 63843762 | 63844196 | − |
| chr10 | 43107051 | 43107190 | + | chr2 | 183441244 | 183441614 | − | chr5 | 63996242 | 63996657 | + |
| chr10 | 43658413 | 43658666 | + | chr2 | 183957414 | 183957604 | + | chr5 | 66319347 | 66319528 | − |
| chr10 | 45811675 | 45811823 | − | chr2 | 184129136 | 184129296 | + | chr5 | 67714062 | 67714500 | − |
| chr10 | 48554478 | 48554888 | + | chr2 | 184843039 | 184843232 | + | chr5 | 67897025 | 67897141 | − |
| chr10 | 48727882 | 48728289 | + | chr2 | 184846126 | 184846253 | + | chr5 | 67897594 | 67897679 | − |
| chr10 | 49068684 | 49068842 | − | chr2 | 184979477 | 184979846 | + | chr5 | 68796502 | 68796756 | + |
| chr10 | 51876611 | 51877053 | + | chr2 | 184980885 | 184980972 | + | chr5 | 68945799 | 68946225 | − |
| chr10 | 53357500 | 53357687 | − | chr2 | 184981515 | 184981755 | + | chr5 | 71815204 | 71815686 | − |
| chr10 | 54065247 | 54065481 | + | chr2 | 185277676 | 185278122 | − | chr5 | 71995383 | 71995537 | − |
| chr10 | 55471943 | 55472231 | + | chr2 | 185307217 | 185307582 | + | chr5 | 71995561 | 71995747 | − |
| chr10 | 55714797 | 55715209 | − | chr2 | 185460713 | 185461100 | − | chr5 | 72539023 | 72539292 | − |
| chr10 | 56216077 | 56216159 | − | chr2 | 185574504 | 185574632 | − | chr5 | 72594349 | 72594770 | + |
| chr10 | 56329617 | 56329845 | + | chr2 | 186079140 | 186079367 | + | chr5 | 72601249 | 72601320 | − |
| chr10 | 57485578 | 57485760 | + | chr2 | 186451490 | 186451837 | − | chr5 | 72657391 | 72657722 | − |
| chr10 | 57486191 | 57486340 | + | chr2 | 186748418 | 186748811 | + | chr5 | 73328738 | 73329014 | − |
| chr10 | 57900619 | 57900934 | + | chr2 | 187823264 | 187823341 | + | chr5 | 73358512 | 73358774 | − |
| chr10 | 58005937 | 58006348 | − | chr2 | 188382074 | 188382558 | + | chr5 | 73359108 | 73359309 | − |
| chr10 | 58589112 | 58589326 | + | chr2 | 189293275 | 189293484 | + | chr5 | 74133586 | 74133996 | + |
| chr10 | 58921653 | 58921950 | + | chr2 | 189294154 | 189294282 | + | chr5 | 74202283 | 74202550 | − |
| chr10 | 58921962 | 58922019 | + | chr2 | 190789445 | 190789917 | + | chr5 | 74260685 | 74261027 | − |
| chr10 | 59438415 | 59438865 | + | chr2 | 191083117 | 191083386 | − | chr5 | 74440903 | 74441362 | + |
| chr10 | 60323017 | 60323473 | − | chr2 | 192347673 | 192347992 | − | chr5 | 74442641 | 74442747 | − |
| chr10 | 61841350 | 61841777 | − | chr2 | 193264921 | 193265186 | − | chr5 | 75746091 | 75746463 | − |
| chr10 | 62130983 | 62131364 | − | chr2 | 193562873 | 193563005 | − | chr5 | 75916794 | 75917197 | + |
| chr10 | 62470103 | 62470521 | − | chr2 | 193563364 | 193563629 | − | chr5 | 75953546 | 75953973 | + |
| chr10 | 62668446 | 62668902 | + | chr2 | 194691751 | 194692108 | + | chr5 | 76754759 | 76755224 | + |
| chr10 | 63017311 | 63017455 | − | chr2 | 197866460 | 197866843 | + | chr5 | 76878385 | 76878626 | + |
| chr10 | 63017533 | 63017625 | − | chr2 | 200284018 | 200284472 | + | chr5 | 77915409 | 77915715 | − |
| chr10 | 63672398 | 63672731 | − | chr2 | 201113530 | 201113693 | − | chr5 | 78554328 | 78554585 | + |
| chr10 | 64271232 | 64271529 | − | chr2 | 201695504 | 201695605 | − | chr5 | 79801267 | 79801467 | − |
| chr10 | 64271547 | 64271939 | + | chr2 | 202671181 | 202671548 | − | chr5 | 80097521 | 80097910 | + |
| chr10 | 64611918 | 64612210 | − | chr2 | 203810151 | 203810375 | − | chr5 | 80628424 | 80628605 | + |
| chr10 | 64863724 | 64864129 | − | chr2 | 203810398 | 203810523 | − | chr5 | 81396625 | 81396707 | − |
| chr10 | 64910312 | 64910448 | + | chr2 | 203969115 | 203969201 | + | chr5 | 81397231 | 81397308 | − |
| chr10 | 64910506 | 64910619 | + | chr2 | 204072608 | 204072898 | − | chr5 | 82149257 | 82149415 | + |
| chr10 | 64981895 | 64982207 | + | chr2 | 204102433 | 204102692 | − | chr5 | 82311383 | 82311825 | + |
| chr10 | 66279406 | 66279773 | + | chr2 | 204388939 | 204389394 | − | chr5 | 82322932 | 82323389 | + |
| chr10 | 67799901 | 67799948 | − | chr2 | 204404529 | 204404927 | − | chr5 | 83465598 | 83466084 | + |
| chr10 | 67800248 | 67800558 | − | chr2 | 204473934 | 204474145 | − | chr5 | 84348046 | 84348288 | − |
| chr10 | 69763189 | 69763412 | − | chr2 | 204501123 | 204501365 | − | chr5 | 84578575 | 84578946 | + |
| chr10 | 69764710 | 69764915 | − | chr2 | 204501801 | 204501873 | − | chr5 | 84589690 | 84589842 | + |
| chr10 | 70592473 | 70592924 | − | chr2 | 205567746 | 205568045 | − | chr5 | 84905073 | 84905458 | + |
| chr10 | 70594233 | 70594508 | + | chr2 | 205899588 | 205900078 | − | chr5 | 84929541 | 84929632 | + |
| chr10 | 70594811 | 70594944 | + | chr2 | 206488425 | 206488771 | − | chr5 | 84931105 | 84931388 | − |
| chr10 | 70899493 | 70899774 | + | chr2 | 207435743 | 207436207 | + | chr5 | 85576351 | 85576494 | + |
| chr10 | 70946239 | 70946639 | − | chr2 | 208545526 | 208545977 | + | chr5 | 85576854 | 85577060 | + |
| chr10 | 71040521 | 71040730 | + | chr2 | 208974467 | 208974766 | − | chr5 | 85938706 | 85938804 | + |
| chr10 | 71076909 | 71077362 | − | chr2 | 210588895 | 210589329 | + | chr5 | 85939110 | 85939457 | + |
| chr10 | 71348784 | 71349083 | − | chr2 | 210882425 | 210882945 | + | chr5 | 86141159 | 86141410 | + |
| chr10 | 71378388 | 71378544 | + | chr2 | 210952883 | 210953244 | + | chr5 | 87127025 | 87127135 | + |
| chr10 | 71378735 | 71378948 | + | chr2 | 211616192 | 211616428 | + | chr5 | 87127218 | 87127436 | + |
| chr10 | 71505064 | 71505282 | − | chr2 | 212030477 | 212030867 | − | chr5 | 87262226 | 87262688 | + |
| chr10 | 71505453 | 71505669 | − | chr2 | 212802265 | 212802738 | + | chr5 | 88494663 | 88494768 | + |
| chr10 | 71923165 | 71923404 | − | chr2 | 212807176 | 212807530 | + | chr5 | 89716154 | 89716244 | − |
| chr10 | 72006801 | 72007078 | + | chr2 | 213726007 | 213726383 | + | chr5 | 90435943 | 90436388 | − |
| chr10 | 72017003 | 72017443 | − | chr2 | 214030717 | 214030990 | + | chr5 | 90715113 | 90715508 | − |
| chr10 | 73628963 | 73629088 | + | chr2 | 214032026 | 214032190 | + | chr5 | 91425188 | 91425517 | + |
| chr10 | 73629859 | 73630040 | + | chr2 | 214509027 | 214509200 | + | chr5 | 91650039 | 91650473 | + |
| chr10 | 73890705 | 73890863 | − | chr2 | 214846531 | 214846782 | − | chr5 | 94195896 | 94196343 | + |
| chr10 | 73891445 | 73891735 | − | chr2 | 215604138 | 215604448 | − | chr5 | 94992481 | 94992894 | + |
| chr10 | 74337098 | 74337523 | − | chr2 | 215777951 | 215777997 | − | chr5 | 95291203 | 95291499 | + |
| chr10 | 75063452 | 75063774 | + | chr2 | 215778266 | 215778693 | − | chr5 | 97417950 | 97418316 | + |
| chr10 | 75159483 | 75159608 | + | chr2 | 216593460 | 216593771 | + | chr5 | 97761037 | 97761544 | + |
| chr10 | 75159762 | 75159912 | + | chr2 | 216916543 | 216916641 | − | chr5 | 97954321 | 97954576 | + |
| chr10 | 75260458 | 75260664 | − | chr2 | 216916931 | 216917268 | − | chr5 | 97956583 | 97956797 | + |
| chr10 | 75261160 | 75261407 | − | chr2 | 217260661 | 217261017 | + | chr5 | 98174037 | 98174180 | − |
| chr10 | 75366632 | 75367021 | + | chr2 | 217560129 | 217560307 | − | chr5 | 99431926 | 99432082 | + |
| chr10 | 75911868 | 75911985 | + | chr2 | 217598822 | 217599152 | + | chr5 | 99703748 | 99704043 | − |
| chr10 | 75912021 | 75912283 | + | chr2 | 217636294 | 217636497 | + | chr5 | 100274621 | 100274721 | + |
| chr10 | 76013805 | 76014027 | + | chr2 | 217636609 | 217636747 | + | chr5 | 100333658 | 100333814 | − |
| chr10 | 76014322 | 76014488 | + | chr2 | 219368710 | 219369173 | − | chr5 | 100618317 | 100618488 | − |
| chr10 | 76746859 | 76747154 | + | chr2 | 219931644 | 219931757 | − | chr5 | 102332729 | 102332981 | − |
| chr10 | 77152342 | 77152818 | − | chr2 | 220977390 | 220977861 | − | chr5 | 102465240 | 102465537 | + |
| chr10 | 77458621 | 77458777 | − | chr2 | 221978704 | 221978898 | + | chr5 | 102628496 | 102628778 | + |
| chr10 | 77458974 | 77459094 | − | chr2 | 221979291 | 221979389 | + | chr5 | 103569340 | 103569784 | + |
| chr10 | 77864896 | 77864965 | + | chr2 | 223153381 | 223153648 | + | chr5 | 104569698 | 104570126 | + |
| chr10 | 78147155 | 78147630 | + | chr2 | 223486664 | 223486916 | + | chr5 | 105949072 | 105949226 | + |
| chr10 | 78172817 | 78173295 | + | chr2 | 224069853 | 224070316 | + | chr5 | 107326414 | 107326724 | − |
| chr10 | 78217508 | 78217983 | + | chr2 | 224604592 | 224605044 | − | chr5 | 107925380 | 107925855 | + |
| chr10 | 78222483 | 78222674 | − | chr2 | 225115713 | 225116134 | + | chr5 | 108448462 | 108448556 | − |
| chr10 | 79224767 | 79225056 | − | chr2 | 225168200 | 225168563 | + | chr5 | 108470910 | 108471153 | + |
| chr10 | 79471167 | 79471410 | + | chr2 | 225271023 | 225271141 | + | chr5 | 108579338 | 108579766 | + |
| chr10 | 81134382 | 81134851 | − | chr2 | 225276255 | 225276520 | − | chr5 | 108702682 | 108702921 | + |
| chr10 | 81576019 | 81576140 | − | chr2 | 225276551 | 225276637 | − | chr5 | 109032393 | 109032535 | + |
| chr10 | 81576443 | 81576677 | − | chr2 | 225288947 | 225289423 | − | chr5 | 109299067 | 109299511 | + |
| chr10 | 81975426 | 81975657 | − | chr2 | 226582271 | 226582613 | + | chr5 | 110383441 | 110383680 | − |
| chr10 | 82898067 | 82898311 | + | chr2 | 226849504 | 226849630 | + | chr5 | 110383983 | 110384219 | − |
| chr10 | 83273938 | 83274229 | − | chr2 | 226849792 | 226849862 | + | chr5 | 112657484 | 112657581 | + |
| chr10 | 83994502 | 83994572 | + | chr2 | 226849928 | 226850049 | + | chr5 | 112657905 | 112658286 | + |
| chr10 | 84990947 | 84991231 | − | chr2 | 227123391 | 227123614 | − | chr5 | 112974697 | 112975124 | + |
| chr10 | 85251413 | 85251734 | + | chr2 | 228581941 | 228582319 | + | chr5 | 113958634 | 113958851 | − |
| chr10 | 86057999 | 86058124 | − | chr2 | 229173792 | 229174099 | + | chr5 | 115072120 | 115072182 | + |
| chr10 | 86080644 | 86081068 | − | chr2 | 230643584 | 230643768 | + | chr5 | 115088085 | 115088553 | + |
| chr10 | 86580539 | 86580887 | − | chr2 | 230643830 | 230643889 | + | chr5 | 116738519 | 116738559 | + |
| chr10 | 86581210 | 86581348 | − | chr2 | 230823753 | 230823989 | + | chr5 | 116738602 | 116738880 | + |
| chr10 | 88130848 | 88131161 | − | chr2 | 231008039 | 231008351 | − | chr5 | 116740634 | 116741076 | − |
| chr10 | 91199807 | 91200271 | − | chr2 | 233214342 | 233214476 | − | chr5 | 116934762 | 116935045 | − |
| chr10 | 93166003 | 93166248 | − | chr2 | 233218993 | 233219118 | − | chr5 | 117107812 | 117108048 | − |
| chr10 | 94368513 | 94368713 | + | chr2 | 233774167 | 233774489 | − | chr5 | 117367932 | 117368270 | + |
| chr10 | 94369121 | 94369183 | + | chr2 | 233962181 | 233962545 | + | chr5 | 119028312 | 119028516 | − |
| chr10 | 94369523 | 94369694 | + | chr2 | 233968234 | 233968501 | − | chr5 | 119651965 | 119652388 | + |
| chr10 | 95731393 | 95731714 | + | chr2 | 234804733 | 234805067 | − | chr5 | 120135896 | 120136312 | + |
| chr10 | 96612690 | 96612768 | + | chr2 | 235308213 | 235308622 | + | chr5 | 120136382 | 120136481 | − |
| chr10 | 96612782 | 96612869 | + | chr2 | 236531982 | 236532266 | − | chr5 | 120264879 | 120265253 | + |
| chr10 | 96624958 | 96625398 | − | chr2 | 236993817 | 236994247 | + | chr5 | 120393373 | 120393798 | − |
| chr10 | 98114421 | 98114645 | − | chr2 | 237182764 | 237183105 | − | chr5 | 120760652 | 120761008 | + |
| chr10 | 101054661 | 101055130 | − | chr2 | 237322710 | 237322986 | + | chr5 | 121257502 | 121257956 | − |
| chr10 | 103464967 | 103465016 | + | chr2 | 237455403 | 237455745 | + | chr5 | 121817962 | 121818394 | − |
| chr10 | 103465321 | 103465707 | + | chr2 | 237546850 | 237547288 | − | chr5 | 122176966 | 122177217 | − |
| chr10 | 103476926 | 103477097 | + | chr2 | 238586842 | 238587160 | − | chr5 | 123692157 | 123692211 | + |
| chr10 | 103708585 | 103708735 | + | chr2 | 238683824 | 238684234 | − | chr5 | 124055497 | 124055807 | − |
| chr10 | 103737416 | 103737746 | − | chr2 | 239634979 | 239635053 | − | chr5 | 125672991 | 125673303 | + |
| chr10 | 103837363 | 103837404 | − | chr2 | 239908888 | 239909007 | + | chr5 | 125673686 | 125673823 | + |
| chr10 | 103843290 | 103843397 | + | chr2 | 239909022 | 239909161 | + | chr5 | 126162779 | 126162960 | + |
| chr10 | 103878779 | 103879189 | − | chr2 | 239947296 | 239947397 | + | chr5 | 129343661 | 129344022 | − |
| chr10 | 104455679 | 104456118 | − | chr2 | 240426156 | 240426337 | − | chr5 | 129856923 | 129857407 | + |
| chr10 | 104481462 | 104481866 | + | chr2 | 240592979 | 240593032 | − | chr5 | 129888023 | 129888501 | + |
| chr10 | 104651739 | 104652043 | + | chr2 | 240796427 | 240796577 | + | chr5 | 130211589 | 130211750 | − |
| chr10 | 104989974 | 104990442 | + | chr2 | 241213468 | 241213513 | − | chr5 | 130776211 | 130776547 | − |
| chr10 | 105604571 | 105604684 | − | chr20 | 1009292 | 1009477 | + | chr5 | 132645545 | 132645865 | − |
| chr10 | 105604699 | 105604764 | − | chr20 | 1013742 | 1013895 | + | chr5 | 133322404 | 133322880 | − |
| chr10 | 105744964 | 105745203 | − | chr20 | 1016970 | 1017194 | + | chr5 | 133441009 | 133441394 | − |
| chr10 | 105826007 | 105826162 | + | chr20 | 1114185 | 1114427 | + | chr5 | 133656076 | 133656554 | − |
| chr10 | 105939971 | 105940283 | + | chr20 | 1195547 | 1195821 | − | chr5 | 133767046 | 133767467 | − |
| chr10 | 106508816 | 106509281 | + | chr20 | 1524245 | 1524331 | + | chr5 | 135420059 | 135420516 | − |
| chr10 | 106900399 | 106900770 | + | chr20 | 1863781 | 1864228 | − | chr5 | 135527423 | 135527489 | − |
| chr10 | 107321906 | 107322306 | − | chr20 | 2050474 | 2050902 | − | chr5 | 135629525 | 135629955 | − |
| chr10 | 107404749 | 107404898 | − | chr20 | 2178405 | 2178478 | − | chr5 | 135669337 | 135669724 | − |
| chr10 | 107554979 | 107555092 | + | chr20 | 2178694 | 2178850 | − | chr5 | 136095061 | 136095494 | + |
| chr10 | 109049866 | 109049997 | + | chr20 | 2624484 | 2624720 | − | chr5 | 138721001 | 138721117 | + |
| chr10 | 109103773 | 109104193 | − | chr20 | 2699966 | 2700342 | + | chr5 | 138722608 | 138722808 | + |
| chr10 | 109104722 | 109104809 | − | chr20 | 2762478 | 2762953 | − | chr5 | 138722858 | 138722951 | + |
| chr10 | 109324813 | 109324948 | + | chr20 | 3254382 | 3254514 | + | chr5 | 138723372 | 138723485 | + |
| chr10 | 109346007 | 109346128 | − | chr20 | 3423242 | 3423545 | + | chr5 | 141265511 | 141265941 | − |
| chr10 | 109617486 | 109617563 | − | chr20 | 3454944 | 3455413 | + | chr5 | 141266294 | 141266398 | − |
| chr10 | 109864036 | 109864341 | − | chr20 | 3825549 | 3825904 | − | chr5 | 142742288 | 142742693 | + |
| chr10 | 111223640 | 111223733 | − | chr20 | 4026066 | 4026536 | − | chr5 | 144575089 | 144575505 | + |
| chr10 | 111224089 | 111224331 | − | chr20 | 4037470 | 4037504 | + | chr5 | 145601763 | 145602210 | − |
| chr10 | 111287868 | 111288066 | + | chr20 | 4047714 | 4047827 | − | chr5 | 145952680 | 145953108 | − |
| chr10 | 111516416 | 111516654 | − | chr20 | 4047827 | 4047986 | − | chr5 | 146994361 | 146994661 | − |
| chr10 | 111631889 | 111632337 | − | chr20 | 4068041 | 4068472 | − | chr5 | 148644470 | 148644931 | − |
| chr10 | 111874966 | 111875226 | − | chr20 | 4109416 | 4109482 | + | chr5 | 150765113 | 150765576 | − |
| chr10 | 111900334 | 111900678 | − | chr20 | 4226322 | 4226753 | − | chr5 | 153167727 | 153167937 | + |
| chr10 | 112143300 | 112143484 | − | chr20 | 4276033 | 4276505 | − | chr5 | 153168125 | 153168166 | + |
| chr10 | 113284519 | 113284947 | − | chr20 | 4301447 | 4301708 | + | chr5 | 153168571 | 153168757 | + |
| chr10 | 113372654 | 113373029 | + | chr20 | 4302965 | 4303412 | − | chr5 | 153323895 | 153324015 | + |
| chr10 | 113742464 | 113742811 | + | chr20 | 4303993 | 4304316 | − | chr5 | 153324293 | 153324527 | + |
| chr10 | 113952308 | 113952393 | − | chr20 | 4305106 | 4305261 | + | chr5 | 153671981 | 153672313 | − |
| chr10 | 114645193 | 114645470 | + | chr20 | 4305346 | 4305525 | + | chr5 | 154058798 | 154059277 | − |
| chr10 | 115177406 | 115177533 | − | chr20 | 4307236 | 4307463 | + | chr5 | 154105298 | 154105558 | − |
| chr10 | 117213250 | 117213608 | + | chr20 | 4307486 | 4307633 | + | chr5 | 154105922 | 154105950 | − |
| chr10 | 117634533 | 117634844 | − | chr20 | 4324760 | 4325237 | + | chr5 | 154372454 | 154372712 | − |
| chr10 | 117861947 | 117862114 | − | chr20 | 4332921 | 4333296 | − | chr5 | 155643140 | 155643578 | + |
| chr10 | 117927572 | 117927887 | + | chr20 | 4383682 | 4383750 | + | chr5 | 155855070 | 155855510 | + |
| chr10 | 118103374 | 118103768 | + | chr20 | 4410260 | 4410704 | + | chr5 | 158020936 | 158021251 | − |
| chr10 | 118732478 | 118732776 | + | chr20 | 4465719 | 4466194 | + | chr5 | 158043643 | 158043992 | − |
| chr10 | 118892818 | 118892950 | − | chr20 | 4540749 | 4541194 | + | chr5 | 159860073 | 159860402 | − |
| chr10 | 118893302 | 118893617 | − | chr20 | 4584438 | 4584861 | + | chr5 | 160260344 | 160260576 | − |
| chr10 | 118956314 | 118956585 | − | chr20 | 4679156 | 4679504 | − | chr5 | 161737564 | 161737855 | − |
| chr10 | 119194483 | 119194890 | + | chr20 | 4738191 | 4738548 | − | chr5 | 162209304 | 162209643 | + |
| chr10 | 119258658 | 119258820 | − | chr20 | 4748315 | 4748423 | − | chr5 | 162456047 | 162456116 | − |
| chr10 | 119258839 | 119258989 | − | chr20 | 4748440 | 4748553 | − | chr5 | 162456197 | 162456284 | − |
| chr10 | 120183971 | 120184329 | + | chr20 | 4764800 | 4765201 | + | chr5 | 163102063 | 163102469 | − |
| chr10 | 120232002 | 120232127 | − | chr20 | 4765512 | 4765558 | + | chr5 | 163136770 | 163136911 | + |
| chr10 | 120232456 | 120232771 | − | chr20 | 4819306 | 4819363 | − | chr5 | 163144377 | 163144592 | + |
| chr10 | 120408647 | 120409003 | + | chr20 | 4819432 | 4819576 | − | chr5 | 163942095 | 163942191 | + |
| chr10 | 120432023 | 120432477 | + | chr20 | 4827920 | 4828180 | − | chr5 | 164189658 | 164190089 | + |
| chr10 | 120539056 | 120539217 | + | chr20 | 4828626 | 4828702 | − | chr5 | 164658597 | 164659035 | + |
| chr10 | 120638998 | 120639403 | + | chr20 | 4838751 | 4839023 | − | chr5 | 165251916 | 165252395 | + |
| chr10 | 121130020 | 121130210 | − | chr20 | 4840668 | 4840802 | − | chr5 | 165529861 | 165530322 | + |
| chr10 | 121130432 | 121130673 | − | chr20 | 4840816 | 4840982 | − | chr5 | 166373328 | 166373488 | + |
| chr10 | 123885037 | 123885214 | − | chr20 | 5030580 | 5030853 | + | chr5 | 166373486 | 166373663 | + |
| chr10 | 124166670 | 124166843 | + | chr20 | 5211060 | 5211169 | − | chr5 | 167007959 | 167008146 | + |
| chr10 | 124208184 | 124208523 | + | chr20 | 5216260 | 5216372 | − | chr5 | 169794198 | 169794399 | + |
| chr10 | 124210466 | 124210587 | + | chr20 | 5216641 | 5216922 | − | chr5 | 169993671 | 169993939 | + |
| chr10 | 124354303 | 124354421 | + | chr20 | 5219958 | 5220390 | + | chr5 | 170739473 | 170739615 | + |
| chr10 | 125377398 | 125377629 | − | chr20 | 5265268 | 5265695 | + | chr5 | 170740038 | 170740246 | + |
| chr10 | 125581186 | 125581355 | + | chr20 | 5310660 | 5310767 | + | chr5 | 171332101 | 171332362 | + |
| chr10 | 125581486 | 125581602 | + | chr20 | 5310779 | 5310835 | + | chr5 | 171332543 | 171332683 | + |
| chr10 | 126871554 | 126871599 | − | chr20 | 5322274 | 5322364 | − | chr5 | 171544826 | 171545117 | − |
| chr10 | 126871916 | 126872293 | − | chr20 | 5688471 | 5688858 | + | chr5 | 171620772 | 171621201 | − |
| chr10 | 127586512 | 127586970 | + | chr20 | 5691060 | 5691192 | − | chr5 | 171633977 | 171634303 | + |
| chr10 | 128172093 | 128172202 | + | chr20 | 5691805 | 5692132 | − | chr5 | 172192367 | 172192451 | − |
| chr10 | 130001218 | 130001352 | + | chr20 | 5705844 | 5706127 | + | chr5 | 173453477 | 173453824 | + |
| chr10 | 130696804 | 130696979 | − | chr20 | 5706291 | 5706469 | + | chr5 | 173748402 | 173748635 | − |
| chr10 | 131499796 | 131500109 | + | chr20 | 5716311 | 5716541 | + | chr5 | 174534347 | 174534760 | + |
| chr10 | 131678070 | 131678196 | − | chr20 | 5730703 | 5730770 | + | chr5 | 174985190 | 174985244 | − |
| chr10 | 131765071 | 131765255 | − | chr20 | 5887221 | 5887666 | − | chr5 | 174985377 | 174985631 | + |
| chr10 | 131812813 | 131813156 | + | chr20 | 5929878 | 5929924 | + | chr5 | 175309217 | 175309521 | + |
| chr10 | 132625413 | 132625530 | − | chr20 | 5930406 | 5930666 | + | chr5 | 175317260 | 175317692 | + |
| chr10_KI270824v1_alt | 81235 | 81487 | − | chr20 | 5970288 | 5970375 | − | chr5 | 176612779 | 176613238 | − |
| chr10_KI270825v1_alt | 103588 | 103657 | + | chr20 | 5970508 | 5970675 | − | chr5 | 176697392 | 176697799 | − |
| chr11 | 1654683 | 1654800 | + | chr20 | 6057546 | 6057880 | − | chr5 | 176902191 | 176902264 | − |
| chr11 | 2651297 | 2651549 | − | chr20 | 6113517 | 6113903 | + | chr5 | 176902428 | 176902761 | − |
| chr11 | 3505122 | 3505425 | − | chr20 | 6133567 | 6133935 | + | chr5 | 180436104 | 180436432 | − |
| chr11 | 3505730 | 3505839 | − | chr20 | 6134311 | 6134371 | + | chr5_GL383531v1_alt | 90842 | 90929 | + |
| chr11 | 3655221 | 3655632 | − | chr20 | 6182754 | 6183159 | − | chr5_GL383531v1_alt | 91010 | 91079 | + |
| chr11 | 4615764 | 4615965 | + | chr20 | 6239758 | 6240147 | + | chr5_GL383532v1_alt | 31106 | 31352 | + |
| chr11 | 4615959 | 4616232 | + | chr20 | 6363569 | 6363653 | − | chr5_GL383532v1_alt | 55396 | 55658 | − |
| chr11 | 6762169 | 6762393 | + | chr20 | 6413209 | 6413505 | − | chr5_KI270794v1_alt | 123884 | 124377 | − |
| chr11 | 6762796 | 6763035 | + | chr20 | 6465737 | 6466204 | + | chr6 | 698795 | 699109 | + |
| chr11 | 7301999 | 7302433 | + | chr20 | 6486282 | 6486501 | − | chr6 | 762831 | 762844 | − |
| chr11 | 8007233 | 8007303 | − | chr20 | 6560674 | 6560807 | + | chr6 | 763297 | 763601 | − |
| chr11 | 8008168 | 8008373 | − | chr20 | 6561452 | 6561621 | + | chr6 | 1327705 | 1328028 | − |
| chr11 | 8178496 | 8178950 | + | chr20 | 6579261 | 6579602 | + | chr6 | 3176212 | 3176454 | − |
| chr11 | 10380441 | 10380695 | − | chr20 | 6648054 | 6648157 | + | chr6 | 4141548 | 4141800 | − |
| chr11 | 10380992 | 10381220 | − | chr20 | 6648484 | 6648638 | + | chr6 | 4204951 | 4205265 | + |
| chr11 | 11219386 | 11219709 | + | chr20 | 6684453 | 6684680 | − | chr6 | 4284301 | 4284746 | − |
| chr11 | 11268935 | 11269253 | − | chr20 | 6684696 | 6684814 | − | chr6 | 4637164 | 4637286 | − |
| chr11 | 11303047 | 11303492 | + | chr20 | 6695284 | 6695475 | − | chr6 | 6397413 | 6397733 | + |
| chr11 | 11385663 | 11385741 | + | chr20 | 6695991 | 6696178 | − | chr6 | 6402467 | 6402848 | + |
| chr11 | 11386182 | 11386434 | + | chr20 | 6757508 | 6757884 | − | chr6 | 6602510 | 6602893 | − |
| chr11 | 11391680 | 11391915 | − | chr20 | 6876014 | 6876198 | + | chr6 | 6834949 | 6835281 | − |
| chr11 | 11412709 | 11413180 | + | chr20 | 6876492 | 6876731 | + | chr6 | 8446818 | 8446980 | − |
| chr11 | 11481318 | 11481777 | − | chr20 | 6901742 | 6902110 | + | chr6 | 8561220 | 8561632 | + |
| chr11 | 11668121 | 11668200 | + | chr20 | 6930942 | 6931327 | + | chr6 | 8682477 | 8682700 | + |
| chr11 | 12002867 | 12003221 | + | chr20 | 7187753 | 7188207 | + | chr6 | 8909120 | 8909242 | − |
| chr11 | 12055734 | 12055872 | − | chr20 | 7387276 | 7387744 | − | chr6 | 8909438 | 8909604 | − |
| chr11 | 12056111 | 12056233 | − | chr20 | 7393716 | 7394029 | − | chr6 | 9491144 | 9491459 | − |
| chr11 | 12103417 | 12103844 | − | chr20 | 7474124 | 7474367 | + | chr6 | 11380239 | 11380402 | − |
| chr11 | 12948983 | 12949058 | − | chr20 | 7547900 | 7548348 | − | chr6 | 11459068 | 11459290 | + |
| chr11 | 13077545 | 13077581 | − | chr20 | 7707299 | 7707746 | − | chr6 | 11459424 | 11459629 | + |
| chr11 | 13077641 | 13077907 | − | chr20 | 7907334 | 7907606 | + | chr6 | 11489988 | 11490381 | − |
| chr11 | 13228923 | 13229257 | − | chr20 | 7930479 | 7930868 | − | chr6 | 11612335 | 11612384 | − |
| chr11 | 14372059 | 14372499 | + | chr20 | 7942441 | 7942675 | + | chr6 | 11658536 | 11658777 | + |
| chr11 | 14702439 | 14702599 | − | chr20 | 7942977 | 7943102 | + | chr6 | 11659080 | 11659127 | + |
| chr11 | 15030566 | 15030938 | + | chr20 | 8065423 | 8065908 | + | chr6 | 11916916 | 11917044 | − |
| chr11 | 15031071 | 15031131 | + | chr20 | 8083252 | 8083741 | + | chr6 | 11917995 | 11918205 | − |
| chr11 | 15057628 | 15058004 | + | chr20 | 8100084 | 8100244 | − | chr6 | 12974712 | 12974973 | − |
| chr11 | 15538498 | 15538945 | + | chr20 | 8156048 | 8156464 | + | chr6 | 14275903 | 14276341 | − |
| chr11 | 15623984 | 15624321 | + | chr20 | 8163047 | 8163433 | − | chr6 | 14428797 | 14428864 | − |
| chr11 | 15733341 | 15733430 | − | chr20 | 8203801 | 8204066 | + | chr6 | 14429272 | 14429670 | − |
| chr11 | 15751418 | 15751823 | + | chr20 | 8290243 | 8290691 | − | chr6 | 18315493 | 18315578 | + |
| chr11 | 19599946 | 19600277 | − | chr20 | 8321887 | 8322266 | + | chr6 | 18789104 | 18789315 | + |
| chr11 | 21256769 | 21256991 | − | chr20 | 8324799 | 8324864 | + | chr6 | 18999190 | 18999520 | + |
| chr11 | 21257063 | 21257163 | − | chr20 | 8551490 | 8551851 | + | chr6 | 18999929 | 18999975 | + |
| chr11 | 21808807 | 21809072 | + | chr20 | 8568764 | 8569252 | − | chr6 | 19178501 | 19178764 | − |
| chr11 | 22040450 | 22040555 | + | chr20 | 8574505 | 8574952 | − | chr6 | 19279299 | 19279518 | + |
| chr11 | 22040557 | 22040760 | + | chr20 | 8622486 | 8622800 | + | chr6 | 19942687 | 19942792 | + |
| chr11 | 22305364 | 22305652 | − | chr20 | 8666075 | 8666345 | + | chr6 | 20089167 | 20089599 | + |
| chr11 | 23415349 | 23415821 | + | chr20 | 8668502 | 8668672 | − | chr6 | 21553330 | 21553762 | − |
| chr11 | 23924351 | 23924492 | − | chr20 | 8668668 | 8668725 | − | chr6 | 21646653 | 21646781 | + |
| chr11 | 23970690 | 23970961 | − | chr20 | 8673870 | 8674087 | + | chr6 | 21662668 | 21662970 | − |
| chr11 | 24230072 | 24230229 | − | chr20 | 8862804 | 8863079 | − | chr6 | 21663271 | 21663403 | − |
| chr11 | 24684544 | 24684863 | + | chr20 | 8893148 | 8893380 | − | chr6 | 21751637 | 21752063 | + |
| chr11 | 24805721 | 24805842 | − | chr20 | 9129184 | 9129558 | + | chr6 | 24261384 | 24261797 | + |
| chr11 | 26061503 | 26061570 | + | chr20 | 9136206 | 9136531 | − | chr6 | 24790326 | 24790596 | + |
| chr11 | 26278610 | 26278846 | + | chr20 | 9193152 | 9193506 | − | chr6 | 26583964 | 26584377 | − |
| chr11 | 26715216 | 26715655 | + | chr20 | 9391626 | 9392038 | + | chr6 | 30314890 | 30315141 | + |
| chr11 | 27083948 | 27084390 | + | chr20 | 9441423 | 9441740 | − | chr6 | 30841188 | 30841534 | − |
| chr11 | 27622651 | 27623002 | − | chr20 | 9492888 | 9493369 | − | chr6 | 30873343 | 30873529 | + |
| chr11 | 28614658 | 28614700 | − | chr20 | 9522610 | 9522742 | − | chr6 | 31131888 | 31131950 | − |
| chr11 | 28615078 | 28615419 | − | chr20 | 9551886 | 9552025 | − | chr6 | 31921810 | 31922217 | − |
| chr11 | 29090125 | 29090224 | + | chr20 | 9553250 | 9553348 | − | chr6 | 32883791 | 32884205 | + |
| chr11 | 29638800 | 29639150 | − | chr20 | 9628695 | 9629063 | + | chr6 | 33600821 | 33600953 | + |
| chr11 | 29898606 | 29898988 | + | chr20 | 9680294 | 9680736 | − | chr6 | 33602003 | 33602233 | + |
| chr11 | 29916528 | 29916922 | − | chr20 | 9686799 | 9687253 | − | chr6 | 33960790 | 33960868 | + |
| chr11 | 30123109 | 30123316 | + | chr20 | 9690374 | 9690734 | − | chr6 | 33960899 | 33960976 | + |
| chr11 | 30720502 | 30720946 | + | chr20 | 9708560 | 9709043 | + | chr6 | 34046388 | 34046657 | − |
| chr11 | 31996232 | 31996375 | − | chr20 | 9756818 | 9757223 | + | chr6 | 34051997 | 34052379 | + |
| chr11 | 32137953 | 32138177 | + | chr20 | 9772830 | 9772978 | + | chr6 | 34518476 | 34518721 | − |
| chr11 | 32229066 | 32229395 | − | chr20 | 9791440 | 9791593 | + | chr6 | 35037415 | 35037763 | − |
| chr11 | 33568743 | 33569163 | + | chr20 | 9822735 | 9822964 | + | chr6 | 36320174 | 36320545 | − |
| chr11 | 33614993 | 33615189 | + | chr20 | 9823715 | 9823840 | + | chr6 | 36630508 | 36630639 | + |
| chr11 | 35365433 | 35365494 | + | chr20 | 9861697 | 9861978 | − | chr6 | 36691900 | 36692175 | − |
| chr11 | 35366261 | 35366726 | + | chr20 | 9866448 | 9866824 | − | chr6 | 36824084 | 36824405 | + |
| chr11 | 35457615 | 35458063 | − | chr20 | 9911748 | 9912052 | − | chr6 | 36842815 | 36842888 | − |
| chr11 | 36310423 | 36310716 | − | chr20 | 9926534 | 9926933 | + | chr6 | 36843167 | 36843557 | − |
| chr11 | 36311020 | 36311071 | − | chr20 | 10008798 | 10008855 | − | chr6 | 37247242 | 37247381 | − |
| chr11 | 36820025 | 36820387 | − | chr20 | 10008986 | 10009072 | − | chr6 | 37247673 | 37247984 | − |
| chr11 | 36969407 | 36969522 | + | chr20 | 10075759 | 10076141 | + | chr6 | 37532738 | 37533181 | − |
| chr11 | 36969739 | 36969794 | + | chr20 | 10078536 | 10078741 | + | chr6 | 37683425 | 37683891 | + |
| chr11 | 37163658 | 37164117 | + | chr20 | 10079249 | 10079479 | + | chr6 | 37709625 | 37709987 | + |
| chr11 | 37264324 | 37264680 | − | chr20 | 10086480 | 10086911 | − | chr6 | 37748718 | 37749210 | + |
| chr11 | 37468412 | 37468578 | − | chr20 | 10093927 | 10094182 | − | chr6 | 37808267 | 37808427 | + |
| chr11 | 38405605 | 38405839 | − | chr20 | 10094605 | 10094840 | − | chr6 | 37808528 | 37808588 | + |
| chr11 | 41874355 | 41874526 | + | chr20 | 10158731 | 10159003 | + | chr6 | 37808601 | 37808657 | + |
| chr11 | 42091548 | 42091692 | − | chr20 | 10311798 | 10312152 | + | chr6 | 38687140 | 38687432 | + |
| chr11 | 42311511 | 42311821 | − | chr20 | 10343471 | 10343874 | + | chr6 | 39046055 | 39046113 | − |
| chr11 | 43505405 | 43505513 | + | chr20 | 10758324 | 10758803 | + | chr6 | 39046427 | 39046722 | − |
| chr11 | 43505940 | 43506182 | + | chr20 | 10777626 | 10778070 | − | chr6 | 39123633 | 39124005 | + |
| chr11 | 43699655 | 43699772 | − | chr20 | 10781931 | 10782372 | − | chr6 | 39292982 | 39293324 | + |
| chr11 | 43699957 | 43700191 | − | chr20 | 10839349 | 10839798 | + | chr6 | 39318494 | 39318979 | + |
| chr11 | 43704038 | 43704119 | − | chr20 | 10863160 | 10863445 | + | chr6 | 39328596 | 39328980 | − |
| chr11 | 43980991 | 43981373 | − | chr20 | 10866042 | 10866371 | − | chr6 | 39401332 | 39401786 | − |
| chr11 | 44377011 | 44377204 | − | chr20 | 10911994 | 10912320 | − | chr6 | 39705633 | 39705958 | + |
| chr11 | 45361382 | 45361833 | − | chr20 | 10947902 | 10948243 | − | chr6 | 39734141 | 39734615 | − |
| chr11 | 45456042 | 45456139 | − | chr20 | 11143319 | 11143777 | + | chr6 | 39897979 | 39898021 | + |
| chr11 | 45456462 | 45456666 | − | chr20 | 11168757 | 11169067 | − | chr6 | 39898038 | 39898303 | − |
| chr11 | 45596813 | 45597201 | + | chr20 | 11177697 | 11178148 | + | chr6 | 39898343 | 39898426 | − |
| chr11 | 47597779 | 47598108 | + | chr20 | 11270355 | 11270564 | − | chr6 | 39899418 | 39899457 | − |
| chr11 | 47598411 | 47598483 | + | chr20 | 11342564 | 11342969 | − | chr6 | 40001302 | 40001419 | + |
| chr11 | 56814093 | 56814405 | + | chr20 | 11583974 | 11584151 | − | chr6 | 40001446 | 40001686 | + |
| chr11 | 56814563 | 56814703 | + | chr20 | 11587181 | 11587651 | − | chr6 | 40033473 | 40033757 | − |
| chr11 | 56832447 | 56832604 | + | chr20 | 11617957 | 11618239 | + | chr6 | 40036627 | 40037065 | − |
| chr11 | 57386735 | 57387106 | − | chr20 | 11723833 | 11724298 | − | chr6 | 40197510 | 40197727 | − |
| chr11 | 58363170 | 58363353 | + | chr20 | 11809990 | 11810193 | − | chr6 | 40197786 | 40197898 | − |
| chr11 | 58394364 | 58394638 | + | chr20 | 11813389 | 11813420 | + | chr6 | 40291258 | 40291611 | − |
| chr11 | 58521123 | 58521233 | + | chr20 | 11813879 | 11814300 | + | chr6 | 40301883 | 40302321 | − |
| chr11 | 58521533 | 58521653 | + | chr20 | 11817055 | 11817463 | − | chr6 | 40420776 | 40421055 | − |
| chr11 | 60697115 | 60697417 | − | chr20 | 11850819 | 11850903 | + | chr6 | 40440608 | 40440943 | + |
| chr11 | 60739387 | 60739792 | − | chr20 | 11879208 | 11879424 | + | chr6 | 40464746 | 40464878 | + |
| chr11 | 60740591 | 60740687 | − | chr20 | 11879425 | 11879664 | + | chr6 | 40465384 | 40465583 | + |
| chr11 | 60740661 | 60740906 | − | chr20 | 11986135 | 11986501 | + | chr6 | 40485790 | 40486257 | − |
| chr11 | 60839674 | 60839923 | − | chr20 | 12012266 | 12012644 | − | chr6 | 40515445 | 40515606 | + |
| chr11 | 61020502 | 61020844 | − | chr20 | 12072548 | 12072975 | + | chr6 | 40515918 | 40516183 | + |
| chr11 | 61254501 | 61254880 | + | chr20 | 12088029 | 12088198 | + | chr6 | 40597643 | 40597939 | − |
| chr11 | 61487351 | 61487770 | − | chr20 | 12199572 | 12200025 | − | chr6 | 40602875 | 40603216 | + |
| chr11 | 62133162 | 62133236 | − | chr20 | 12247781 | 12248228 | − | chr6 | 40603621 | 40603740 | + |
| chr11 | 62990316 | 62990443 | + | chr20 | 12286264 | 12286669 | − | chr6 | 40627508 | 40627792 | + |
| chr11 | 64461011 | 64461078 | − | chr20 | 12592886 | 12593306 | + | chr6 | 40717774 | 40718012 | − |
| chr11 | 64516836 | 64516940 | + | chr20 | 12608668 | 12608999 | + | chr6 | 40735037 | 40735374 | − |
| chr11 | 64517223 | 64517548 | − | chr20 | 12609147 | 12609301 | + | chr6 | 40744057 | 40744261 | − |
| chr11 | 64548170 | 64548471 | − | chr20 | 12646739 | 12647103 | − | chr6 | 40787810 | 40788095 | − |
| chr11 | 65339920 | 65340005 | − | chr20 | 12661977 | 12662406 | + | chr6 | 40800324 | 40800728 | − |
| chr11 | 65708022 | 65708190 | − | chr20 | 12669565 | 12670004 | + | chr6 | 40805836 | 40805961 | + |
| chr11 | 66696993 | 66697315 | + | chr20 | 12736310 | 12736532 | + | chr6 | 40805987 | 40806157 | + |
| chr11 | 67097108 | 67097446 | + | chr20 | 12753591 | 12753814 | + | chr6 | 40935123 | 40935396 | − |
| chr11 | 67877842 | 67878146 | − | chr20 | 12782626 | 12783114 | − | chr6 | 40957303 | 40957742 | − |
| chr11 | 67878453 | 67878562 | − | chr20 | 12821635 | 12822056 | − | chr6 | 41002866 | 41003235 | + |
| chr11 | 68224934 | 68225225 | − | chr20 | 12836054 | 12836235 | + | chr6 | 41193257 | 41193599 | + |
| chr11 | 68225997 | 68226229 | + | chr20 | 12837695 | 12837815 | − | chr6 | 41277760 | 41277900 | + |
| chr11 | 69035396 | 69035652 | + | chr20 | 12837817 | 12837983 | − | chr6 | 41278226 | 41278281 | + |
| chr11 | 69531653 | 69531818 | + | chr20 | 12895353 | 12895736 | + | chr6 | 41537001 | 41537453 | + |
| chr11 | 69802722 | 69802928 | + | chr20 | 12916448 | 12916900 | − | chr6 | 41538363 | 41538761 | − |
| chr11 | 70086589 | 70086911 | + | chr20 | 12965303 | 12965746 | − | chr6 | 41659973 | 41660110 | − |
| chr11 | 70892805 | 70893055 | + | chr20 | 13005259 | 13005636 | − | chr6 | 41708335 | 41708428 | + |
| chr11 | 71716926 | 71717035 | + | chr20 | 13014558 | 13014943 | − | chr6 | 41708526 | 41708691 | + |
| chr11 | 71717341 | 71717656 | + | chr20 | 13026707 | 13027219 | + | chr6 | 41952229 | 41952712 | − |
| chr11 | 72471174 | 72471552 | + | chr20 | 13100881 | 13101196 | − | chr6 | 42480221 | 42480298 | + |
| chr11 | 73258697 | 73259031 | + | chr20 | 13180994 | 13181444 | + | chr6 | 42480591 | 42480830 | + |
| chr11 | 74474811 | 74475182 | − | chr20 | 13195283 | 13195727 | + | chr6 | 42736012 | 42736253 | − |
| chr11 | 74475544 | 74475600 | − | chr20 | 13205784 | 13206133 | − | chr6 | 43831697 | 43832005 | − |
| chr11 | 74986899 | 74987031 | − | chr20 | 13230938 | 13231125 | + | chr6 | 44549486 | 44549949 | + |
| chr11 | 76561602 | 76561994 | + | chr20 | 13296168 | 13296371 | + | chr6 | 47096208 | 47096406 | − |
| chr11 | 76600111 | 76600490 | − | chr20 | 13297286 | 13297512 | + | chr6 | 47364525 | 47364810 | − |
| chr11 | 79021344 | 79021723 | − | chr20 | 13304481 | 13304979 | + | chr6 | 47415887 | 47416215 | + |
| chr11 | 79409781 | 79409946 | + | chr20 | 13368479 | 13368693 | − | chr6 | 47416974 | 47417042 | + |
| chr11 | 79410161 | 79410292 | + | chr20 | 13385589 | 13385895 | − | chr6 | 49852472 | 49852593 | + |
| chr11 | 80085341 | 80085726 | + | chr20 | 13683769 | 13684193 | + | chr6 | 51741394 | 51741824 | + |
| chr11 | 80168762 | 80169216 | − | chr20 | 13836526 | 13836987 | − | chr6 | 53624473 | 53624933 | + |
| chr11 | 81839021 | 81839221 | − | chr20 | 13841333 | 13841636 | + | chr6 | 54237095 | 54237455 | − |
| chr11 | 82506078 | 82506369 | − | chr20 | 13841934 | 13842070 | + | chr6 | 54269152 | 54269379 | − |
| chr11 | 83144051 | 83144257 | − | chr20 | 14409894 | 14410127 | + | chr6 | 54391310 | 54391706 | − |
| chr11 | 83144560 | 83144799 | − | chr20 | 14410710 | 14410910 | + | chr6 | 55001067 | 55001204 | + |
| chr11 | 85364217 | 85364689 | + | chr20 | 14451011 | 14451159 | − | chr6 | 55204074 | 55204486 | + |
| chr11 | 85407535 | 85408001 | − | chr20 | 14451461 | 14451802 | − | chr6 | 57199251 | 57199443 | + |
| chr11 | 85842209 | 85842642 | + | chr20 | 14529911 | 14530324 | − | chr6 | 57267285 | 57267467 | − |
| chr11 | 86183546 | 86184000 | − | chr20 | 14544600 | 14545031 | − | chr6 | 57668819 | 57669163 | + |
| chr11 | 88239095 | 88239467 | − | chr20 | 14548766 | 14549137 | − | chr6 | 60568133 | 60568498 | + |
| chr11 | 90409135 | 90409539 | + | chr20 | 14653849 | 14654062 | + | chr6 | 61855739 | 61855986 | + |
| chr11 | 90413024 | 90413312 | + | chr20 | 14704346 | 14704582 | + | chr6 | 61989751 | 61990188 | + |
| chr11 | 90886250 | 90886627 | + | chr20 | 14725550 | 14725952 | − | chr6 | 62266238 | 62266663 | + |
| chr11 | 91133279 | 91133530 | + | chr20 | 14783618 | 14784090 | − | chr6 | 62266998 | 62267063 | + |
| chr11 | 91629937 | 91630091 | + | chr20 | 14817597 | 14818016 | − | chr6 | 64656447 | 64656802 | − |
| chr11 | 92440168 | 92440530 | − | chr20 | 15030100 | 15030263 | + | chr6 | 65812680 | 65812914 | + |
| chr11 | 93341051 | 93341123 | − | chr20 | 15030411 | 15030593 | + | chr6 | 65876278 | 65876532 | + |
| chr11 | 93342189 | 93342547 | − | chr20 | 15061274 | 15061618 | + | chr6 | 67399124 | 67399450 | + |
| chr11 | 94403501 | 94403931 | − | chr20 | 15090841 | 15091300 | + | chr6 | 67451880 | 67452284 | + |
| chr11 | 95542764 | 95542942 | + | chr20 | 15237534 | 15237999 | − | chr6 | 68257403 | 68257765 | − |
| chr11 | 95547739 | 95547943 | + | chr20 | 15248857 | 15249145 | − | chr6 | 70391428 | 70391851 | + |
| chr11 | 95964961 | 95965285 | − | chr20 | 15407090 | 15407506 | + | chr6 | 72062527 | 72062611 | − |
| chr11 | 96299413 | 96299893 | + | chr20 | 15409556 | 15409769 | − | chr6 | 72063100 | 72063398 | − |
| chr11 | 96798522 | 96798944 | + | chr20 | 15419019 | 15419374 | − | chr6 | 74498343 | 74498686 | + |
| chr11 | 96825918 | 96826380 | − | chr20 | 15466991 | 15467426 | + | chr6 | 75522557 | 75523009 | − |
| chr11 | 97887547 | 97887640 | + | chr20 | 15477508 | 15477661 | − | chr6 | 75995752 | 75995914 | + |
| chr11 | 97887821 | 97887849 | + | chr20 | 15478180 | 15478393 | − | chr6 | 76190306 | 76190391 | − |
| chr11 | 97887894 | 97888129 | + | chr20 | 15491413 | 15491888 | − | chr6 | 76191434 | 76191526 | − |
| chr11 | 98949123 | 98949368 | + | chr20 | 15585079 | 15585187 | + | chr6 | 76305413 | 76305826 | + |
| chr11 | 98949511 | 98949673 | + | chr20 | 15585496 | 15585861 | + | chr6 | 76306357 | 76306442 | + |
| chr11 | 100483420 | 100483798 | + | chr20 | 15804693 | 15805162 | + | chr6 | 76557191 | 76557266 | − |
| chr11 | 101719313 | 101719683 | + | chr20 | 15814148 | 15814635 | + | chr6 | 76558309 | 76558398 | − |
| chr11 | 101752903 | 101753181 | − | chr20 | 15850099 | 15850163 | − | chr6 | 76558597 | 76558743 | − |
| chr11 | 101753258 | 101753297 | − | chr20 | 15850175 | 15850501 | − | chr6 | 77034063 | 77034470 | − |
| chr11 | 101759884 | 101760069 | − | chr20 | 15880378 | 15880861 | − | chr6 | 77589706 | 77590089 | + |
| chr11 | 101760413 | 101760747 | − | chr20 | 15887427 | 15887656 | + | chr6 | 78871836 | 78872353 | + |
| chr11 | 101761099 | 101761220 | − | chr20 | 15887977 | 15888177 | + | chr6 | 80508689 | 80509019 | − |
| chr11 | 101884732 | 101884798 | − | chr20 | 15921722 | 15922180 | − | chr6 | 81595786 | 81596246 | + |
| chr11 | 103638207 | 103638624 | + | chr20 | 15960461 | 15960772 | − | chr6 | 82259410 | 82259581 | + |
| chr11 | 104025473 | 104025587 | − | chr20 | 16023502 | 16024038 | − | chr6 | 82259626 | 82259741 | + |
| chr11 | 104208012 | 104208383 | − | chr20 | 16173289 | 16173372 | − | chr6 | 82787507 | 82787615 | + |
| chr11 | 104656734 | 104657192 | + | chr20 | 16173708 | 16173783 | − | chr6 | 83813738 | 83814142 | − |
| chr11 | 105880956 | 105881437 | − | chr20 | 16206670 | 16207112 | − | chr6 | 87972618 | 87973088 | + |
| chr11 | 107211849 | 107212168 | + | chr20 | 16620553 | 16620677 | + | chr6 | 88108744 | 88109090 | + |
| chr11 | 109411815 | 109411955 | − | chr20 | 16620690 | 16620864 | + | chr6 | 88109636 | 88109986 | + |
| chr11 | 109412238 | 109412720 | + | chr20 | 16629293 | 16629693 | − | chr6 | 88194508 | 88194822 | + |
| chr11 | 109441695 | 109441975 | + | chr20 | 16639931 | 16640165 | + | chr6 | 88486497 | 88486908 | + |
| chr11 | 109441973 | 109442080 | + | chr20 | 16647536 | 16647598 | + | chr6 | 88487213 | 88487267 | + |
| chr11 | 109504713 | 109505092 | − | chr20 | 16653913 | 16654188 | + | chr6 | 89198524 | 89198898 | + |
| chr11 | 110776196 | 110776622 | − | chr20 | 16654372 | 16654496 | + | chr6 | 90320843 | 90320969 | + |
| chr11 | 111001566 | 111002031 | − | chr20 | 16690564 | 16690655 | − | chr6 | 90396292 | 90396376 | − |
| chr11 | 112561498 | 112561902 | + | chr20 | 16690958 | 16691280 | − | chr6 | 90601328 | 90601799 | + |
| chr11 | 112632146 | 112632452 | − | chr20 | 16763889 | 16764291 | + | chr6 | 90958535 | 90958809 | + |
| chr11 | 113519261 | 113519570 | − | chr20 | 16929349 | 16929740 | − | chr6 | 91746538 | 91746989 | − |
| chr11 | 114290447 | 114290548 | − | chr20 | 17070795 | 17071208 | − | chr6 | 92286217 | 92286466 | − |
| chr11 | 116172792 | 116173269 | − | chr20 | 17075302 | 17075437 | − | chr6 | 93044568 | 93044899 | + |
| chr11 | 120607231 | 120607342 | − | chr20 | 17103957 | 17104426 | + | chr6 | 94552989 | 94553240 | + |
| chr11 | 120614159 | 120614249 | − | chr20 | 17143588 | 17143760 | + | chr6 | 98186590 | 98186768 | + |
| chr11 | 120614279 | 120614379 | − | chr20 | 17149239 | 17149647 | − | chr6 | 98432939 | 98433107 | − |
| chr11 | 122515571 | 122515640 | − | chr20 | 17237650 | 17238120 | − | chr6 | 98746523 | 98746836 | + |
| chr11 | 122515873 | 122516242 | − | chr20 | 17240342 | 17240600 | − | chr6 | 99216815 | 99217258 | − |
| chr11 | 123675637 | 123675850 | + | chr20 | 17316440 | 17316581 | + | chr6 | 101222037 | 101222139 | + |
| chr11 | 124471796 | 124471946 | + | chr20 | 17320482 | 17320782 | − | chr6 | 101232094 | 101232225 | + |
| chr11 | 124499869 | 124500319 | + | chr20 | 17323824 | 17324244 | + | chr6 | 101232268 | 101232558 | + |
| chr11 | 124831617 | 124831894 | + | chr20 | 17373094 | 17373413 | − | chr6 | 103054132 | 103054505 | + |
| chr11 | 124941514 | 124941802 | − | chr20 | 17374542 | 17374757 | − | chr6 | 103500861 | 103501286 | − |
| chr11 | 125723500 | 125723574 | − | chr20 | 17374856 | 17374913 | − | chr6 | 103553350 | 103553535 | − |
| chr11 | 126086170 | 126086631 | − | chr20 | 17385637 | 17386079 | − | chr6 | 105451435 | 105451787 | + |
| chr11 | 126548304 | 126548704 | + | chr20 | 17444590 | 17445031 | − | chr6 | 106381493 | 106381897 | − |
| chr11 | 126714533 | 126714831 | + | chr20 | 17480584 | 17481053 | − | chr6 | 106724983 | 106725151 | + |
| chr11 | 126869139 | 126869486 | + | chr20 | 17488600 | 17488847 | + | chr6 | 109689312 | 109689756 | + |
| chr11 | 131072192 | 131072418 | − | chr20 | 17490247 | 17490321 | + | chr6 | 111883736 | 111883991 | − |
| chr11 | 131072396 | 131072560 | − | chr20 | 17490801 | 17491183 | − | chr6 | 111884434 | 111884513 | − |
| chr11 | 131177433 | 131177797 | − | chr20 | 17493222 | 17493624 | + | chr6 | 112715835 | 112716074 | − |
| chr11 | 133243725 | 133243799 | − | chr20 | 17513673 | 17514127 | − | chr6 | 113091006 | 113091418 | + |
| chr11 | 133243818 | 133244099 | − | chr20 | 17521681 | 17522021 | − | chr6 | 113343728 | 113344148 | − |
| chr11_JH159137v1_alt | 131935 | 132247 | + | chr20 | 17525521 | 17525642 | + | chr6 | 114609786 | 114609951 | + |
| chr11_JH159137v1_alt | 132405 | 132545 | + | chr20 | 17544021 | 17544316 | − | chr6 | 115095764 | 115096269 | − |
| chr11_JH159137v1_alt | 150289 | 150446 | + | chr20 | 17684005 | 17684248 | − | chr6 | 116297086 | 116297165 | + |
| chr11_KI270830v1_alt | 136930 | 137037 | + | chr20 | 17688832 | 17689242 | − | chr6 | 116297218 | 116297511 | + |
| chr11_KI270903v1_alt | 128710 | 128817 | + | chr20 | 17697573 | 17697741 | − | chr6 | 119097437 | 119097510 | − |
| chr12 | 122511 | 122801 | − | chr20 | 17698468 | 17698758 | − | chr6 | 119100192 | 119100465 | − |
| chr12 | 937726 | 938098 | + | chr20 | 17709926 | 17710193 | − | chr6 | 120211736 | 120211842 | − |
| chr12 | 974209 | 974311 | + | chr20 | 17736943 | 17737397 | + | chr6 | 120211843 | 120212273 | + |
| chr12 | 976910 | 977003 | − | chr20 | 17741175 | 17741618 | − | chr6 | 120212274 | 120212343 | − |
| chr12 | 977646 | 977879 | − | chr20 | 17754809 | 17755141 | + | chr6 | 120212401 | 120212736 | − |
| chr12 | 977896 | 978008 | − | chr20 | 17771121 | 17771397 | + | chr6 | 120869223 | 120869448 | − |
| chr12 | 1570123 | 1570442 | − | chr20 | 17775999 | 17776256 | + | chr6 | 122567084 | 122567525 | + |
| chr12 | 5719123 | 5719382 | + | chr20 | 17777068 | 17777164 | + | chr6 | 123801296 | 123801597 | − |
| chr12 | 5719757 | 5719998 | + | chr20 | 17797759 | 17797804 | − | chr6 | 123849260 | 123849727 | − |
| chr12 | 6128252 | 6128612 | − | chr20 | 17798174 | 17798630 | + | chr6 | 125403185 | 125403427 | + |
| chr12 | 8313243 | 8313551 | − | chr20 | 17810150 | 17810250 | + | chr6 | 127376736 | 127377061 | − |
| chr12 | 8313859 | 8313968 | − | chr20 | 17810432 | 17810513 | + | chr6 | 128741835 | 128742310 | + |
| chr12 | 10305344 | 10305704 | − | chr20 | 17841278 | 17841523 | − | chr6 | 130111085 | 130111488 | − |
| chr12 | 14272013 | 14272422 | − | chr20 | 17867585 | 17867968 | − | chr6 | 130232304 | 130232448 | + |
| chr12 | 15020801 | 15021029 | − | chr20 | 17882986 | 17883138 | + | chr6 | 131353951 | 131354318 | − |
| chr12 | 17778558 | 17778819 | + | chr20 | 17890754 | 17890845 | − | chr6 | 133783870 | 133783951 | + |
| chr12 | 17974253 | 17974504 | − | chr20 | 17926273 | 17926527 | + | chr6 | 134346556 | 134346644 | − |
| chr12 | 18927108 | 18927367 | + | chr20 | 17933278 | 17933376 | − | chr6 | 134346702 | 134346879 | − |
| chr12 | 20154806 | 20155014 | + | chr20 | 18019140 | 18019450 | + | chr6 | 135104948 | 135105193 | − |
| chr12 | 20360672 | 20360713 | − | chr20 | 18019754 | 18019807 | + | chr6 | 135877657 | 135878135 | − |
| chr12 | 22947292 | 22947726 | + | chr20 | 18025576 | 18026018 | − | chr6 | 136337562 | 136337903 | − |
| chr12 | 25815224 | 25815371 | + | chr20 | 18037731 | 18037953 | + | chr6 | 140016721 | 140017193 | − |
| chr12 | 25815676 | 25815960 | + | chr20 | 18092339 | 18092491 | + | chr6 | 141211925 | 141212235 | − |
| chr12 | 25816530 | 25816577 | + | chr20 | 18092794 | 18092899 | + | chr6 | 141488304 | 141488615 | − |
| chr12 | 26309749 | 26309951 | + | chr20 | 18400145 | 18400673 | − | chr6 | 141665197 | 141665664 | − |
| chr12 | 26774897 | 26775150 | + | chr20 | 18733142 | 18733356 | − | chr6 | 142970614 | 142970913 | + |
| chr12 | 27111045 | 27111186 | − | chr20 | 18807669 | 18807895 | + | chr6 | 142971267 | 142971339 | + |
| chr12 | 27195728 | 27196165 | + | chr20 | 18808171 | 18808314 | + | chr6 | 142972100 | 142972225 | + |
| chr12 | 27398060 | 27398196 | + | chr20 | 18820672 | 18820936 | − | chr6 | 144360512 | 144360955 | + |
| chr12 | 27628779 | 27628977 | − | chr20 | 18821400 | 18821435 | − | chr6 | 145138007 | 145138460 | + |
| chr12 | 27859668 | 27859990 | + | chr20 | 18854933 | 18855235 | + | chr6 | 145753977 | 145754387 | + |
| chr12 | 29460502 | 29460872 | + | chr20 | 18896620 | 18896808 | − | chr6 | 145756586 | 145756793 | + |
| chr12 | 30764636 | 30764700 | + | chr20 | 18899862 | 18900168 | + | chr6 | 145757110 | 145757149 | + |
| chr12 | 32031146 | 32031404 | − | chr20 | 18904203 | 18904489 | + | chr6 | 146440534 | 146441009 | − |
| chr12 | 39723950 | 39724247 | + | chr20 | 18985190 | 18985597 | − | chr6 | 146850513 | 146850978 | − |
| chr12 | 39724411 | 39724599 | + | chr20 | 18993511 | 18993736 | + | chr6 | 152363607 | 152363952 | + |
| chr12 | 39938803 | 39939278 | + | chr20 | 19095608 | 19095751 | + | chr6 | 152364329 | 152364435 | + |
| chr12 | 45685238 | 45685458 | + | chr20 | 19119930 | 19120327 | − | chr6 | 153426100 | 153426339 | − |
| chr12 | 59513662 | 59514057 | + | chr20 | 19251406 | 19251692 | − | chr6 | 153428766 | 153428928 | − |
| chr12 | 59987543 | 59987961 | + | chr20 | 19278008 | 19278460 | + | chr6 | 153945368 | 153945502 | − |
| chr12 | 61526104 | 61526556 | + | chr20 | 19279774 | 19279923 | − | chr6 | 154233708 | 154233926 | + |
| chr12 | 61528757 | 61529207 | + | chr20 | 19280346 | 19280449 | − | chr6 | 154234208 | 154234483 | + |
| chr12 | 67966581 | 67967007 | − | chr20 | 19280655 | 19280815 | − | chr6 | 154323068 | 154323609 | + |
| chr12 | 68177150 | 68177480 | − | chr20 | 19298485 | 19298610 | − | chr6 | 154525839 | 154526074 | − |
| chr12 | 70166254 | 70166501 | + | chr20 | 19298770 | 19299041 | − | chr6 | 155579405 | 155579865 | − |
| chr12 | 72103687 | 72103846 | − | chr20 | 19317879 | 19318324 | + | chr6 | 155825870 | 155826201 | + |
| chr12 | 72103986 | 72104132 | − | chr20 | 19334650 | 19334994 | − | chr6 | 156057914 | 156058311 | + |
| chr12 | 72696627 | 72696916 | + | chr20 | 19344406 | 19344809 | − | chr6 | 156402133 | 156402596 | − |
| chr12 | 72697221 | 72697254 | + | chr20 | 19349349 | 19349802 | + | chr6 | 156440284 | 156440724 | + |
| chr12 | 72715268 | 72715666 | + | chr20 | 19359794 | 19360062 | − | chr6 | 157375415 | 157375544 | + |
| chr12 | 73337596 | 73338064 | − | chr20 | 19371027 | 19371379 | − | chr6 | 157376076 | 157376232 | + |
| chr12 | 76888885 | 76889216 | − | chr20 | 19374793 | 19375178 | + | chr6 | 159421517 | 159421613 | − |
| chr12 | 77578314 | 77578540 | + | chr20 | 19401610 | 19402056 | + | chr6 | 159915671 | 159915810 | − |
| chr12 | 78240330 | 78240501 | − | chr20 | 19421492 | 19421587 | − | chr6 | 164914747 | 164914825 | − |
| chr12 | 80646472 | 80646691 | − | chr20 | 19421926 | 19422100 | − | chr6 | 165024517 | 165024893 | − |
| chr12 | 82151090 | 82151459 | − | chr20 | 19445418 | 19445847 | − | chr6 | 166992673 | 166992940 | − |
| chr12 | 85712444 | 85712911 | + | chr20 | 19455377 | 19455828 | + | chr6 | 167102303 | 167102631 | − |
| chr12 | 86447990 | 86448368 | + | chr20 | 19479474 | 19479779 | − | chr6 | 168766924 | 168767020 | − |
| chr12 | 86750538 | 86750871 | − | chr20 | 19505461 | 19505852 | − | chr6_GL000250v2_alt | 4188619 | 4189029 | + |
| chr12 | 88664267 | 88664586 | + | chr20 | 19510910 | 19511113 | + | chr6_GL000251v2_alt | 1794564 | 1794815 | + |
| chr12 | 90421521 | 90421669 | − | chr20 | 19511138 | 19511262 | + | chr6_GL000251v2_alt | 2320927 | 2321273 | − |
| chr12 | 90520398 | 90520693 | − | chr20 | 19514178 | 19514381 | + | chr6_GL000251v2_alt | 2353117 | 2353303 | + |
| chr12 | 92294608 | 92295019 | + | chr20 | 19523848 | 19524253 | + | chr6_GL000251v2_alt | 2614279 | 2614341 | − |
| chr12 | 92584766 | 92584875 | + | chr20 | 19525318 | 19525640 | − | chr6_GL000251v2_alt | 3399237 | 3399644 | − |
| chr12 | 96543203 | 96543407 | − | chr20 | 19571910 | 19572055 | − | chr6_GL000251v2_alt | 4295990 | 4296404 | + |
| chr12 | 98218661 | 98219066 | + | chr20 | 19572370 | 19572676 | − | chr6_GL000252v2_alt | 1570731 | 1570982 | + |
| chr12 | 98219616 | 98219667 | + | chr20 | 19589093 | 19589552 | − | chr6_GL000252v2_alt | 2097449 | 2097795 | − |
| chr12 | 100679944 | 100680297 | + | chr20 | 19610529 | 19610987 | − | chr6_GL000252v2_alt | 2129624 | 2129810 | + |
| chr12 | 101502173 | 101502555 | + | chr20 | 19646933 | 19647358 | + | chr6_GL000252v2_alt | 2391242 | 2391304 | − |
| chr12 | 101556216 | 101556400 | − | chr20 | 19708944 | 19709254 | − | chr6_GL000252v2_alt | 3169589 | 3169996 | − |
| chr12 | 102803147 | 102803446 | + | chr20 | 19783557 | 19784001 | − | chr6_GL000252v2_alt | 4127315 | 4127729 | + |
| chr12 | 102803918 | 102804077 | + | chr20 | 19805169 | 19805384 | − | chr6_GL000253v2_alt | 1625114 | 1625365 | + |
| chr12 | 102821393 | 102821817 | − | chr20 | 19811137 | 19811567 | − | chr6_GL000253v2_alt | 2151432 | 2151778 | − |
| chr12 | 103554450 | 103554517 | − | chr20 | 19859853 | 19860231 | − | chr6_GL000253v2_alt | 2183585 | 2183771 | + |
| chr12 | 103554921 | 103555072 | − | chr20 | 19863332 | 19863738 | + | chr6_GL000253v2_alt | 2442412 | 2442474 | − |
| chr12 | 103555737 | 103555844 | − | chr20 | 19871396 | 19871482 | − | chr6_GL000253v2_alt | 3227025 | 3227432 | − |
| chr12 | 103555898 | 103556054 | − | chr20 | 19874551 | 19874705 | + | chr6_GL000253v2_alt | 4303144 | 4303558 | + |
| chr12 | 103557475 | 103557812 | + | chr20 | 19915124 | 19915545 | − | chr6_GL000254v2_alt | 2185268 | 2185614 | − |
| chr12 | 103623020 | 103623259 | − | chr20 | 19982689 | 19983088 | + | chr6_GL000254v2_alt | 2217497 | 2217683 | + |
| chr12 | 105267179 | 105267312 | − | chr20 | 20101979 | 20102146 | + | chr6_GL000254v2_alt | 2476024 | 2476086 | − |
| chr12 | 105267643 | 105267767 | − | chr20 | 20102163 | 20102279 | + | chr6_GL000254v2_alt | 3263896 | 3264303 | − |
| chr12 | 105411664 | 105412107 | − | chr20 | 20102292 | 20102483 | + | chr6_GL000254v2_alt | 4182822 | 4183232 | + |
| chr12 | 105746392 | 105746835 | + | chr20 | 20102690 | 20102811 | + | chr6_GL000255v2_alt | 1569962 | 1570213 | + |
| chr12 | 105748509 | 105748809 | + | chr20 | 20160834 | 20161121 | + | chr6_GL000255v2_alt | 2096292 | 2096638 | − |
| chr12 | 106021593 | 106021785 | + | chr20 | 20736950 | 20737416 | − | chr6_GL000255v2_alt | 2128516 | 2128702 | + |
| chr12 | 106205186 | 106205625 | − | chr20 | 20745566 | 20745778 | − | chr6_GL000255v2_alt | 2389913 | 2389975 | − |
| chr12 | 107428341 | 107428743 | + | chr20 | 20771283 | 20771412 | + | chr6_GL000255v2_alt | 3177784 | 3178191 | − |
| chr12 | 107433624 | 107433764 | − | chr20 | 20773347 | 20773633 | + | chr6_GL000255v2_alt | 4078078 | 4078492 | + |
| chr12 | 107434121 | 107434411 | − | chr20 | 20800028 | 20800248 | − | chr6_GL000256v2_alt | 1613345 | 1613596 | + |
| chr12 | 108048100 | 108048530 | − | chr20 | 20804709 | 20805011 | − | chr6_GL000256v2_alt | 3223048 | 3223455 | − |
| chr12 | 108055936 | 108056393 | + | chr20 | 20826817 | 20827257 | − | chr6_GL000256v2_alt | 4283008 | 4283418 | + |
| chr12 | 108112636 | 108112928 | − | chr20 | 20833133 | 20833393 | + | chr6_KI270800v1_alt | 37867 | 38101 | + |
| chr12 | 108189236 | 108189515 | − | chr20 | 20904410 | 20904723 | − | chr6_KI270800v1_alt | 108053 | 108307 | + |
| chr12 | 108216545 | 108216864 | − | chr20 | 20914762 | 20915258 | − | chr7 | 1588239 | 1588328 | − |
| chr12 | 108386076 | 108386531 | − | chr20 | 20921136 | 20921178 | − | chr7 | 1588792 | 1588945 | − |
| chr12 | 108388378 | 108388812 | + | chr20 | 20926216 | 20926298 | − | chr7 | 2875011 | 2875118 | − |
| chr12 | 108452192 | 108452644 | − | chr20 | 20926836 | 20926983 | − | chr7 | 2996914 | 2997177 | + |
| chr12 | 108475587 | 108475998 | + | chr20 | 20951469 | 20951883 | + | chr7 | 3064205 | 3064315 | + |
| chr12 | 108921350 | 108921763 | − | chr20 | 20990953 | 20991003 | − | chr7 | 3064330 | 3064483 | + |
| chr12 | 108949731 | 108949913 | + | chr20 | 20991352 | 20991464 | − | chr7 | 3066128 | 3066227 | − |
| chr12 | 108971582 | 108971756 | − | chr20 | 20991632 | 20991746 | − | chr7 | 3069605 | 3069966 | + |
| chr12 | 109645934 | 109646336 | − | chr20 | 21016182 | 21016479 | + | chr7 | 3071505 | 3071617 | + |
| chr12 | 110080122 | 110080265 | + | chr20 | 21044081 | 21044440 | − | chr7 | 3099591 | 3099726 | − |
| chr12 | 110080550 | 110080661 | + | chr20 | 21046501 | 21046930 | + | chr7 | 3170328 | 3170565 | + |
| chr12 | 110766798 | 110766964 | + | chr20 | 21052515 | 21052979 | − | chr7 | 3885938 | 3886358 | − |
| chr12 | 110767219 | 110767417 | + | chr20 | 21095712 | 21096127 | + | chr7 | 3934744 | 3935010 | − |
| chr12 | 110966894 | 110967302 | + | chr20 | 21108599 | 21109006 | − | chr7 | 4138897 | 4139273 | + |
| chr12 | 111013326 | 111013718 | + | chr20 | 21112373 | 21112452 | + | chr7 | 4375029 | 4375416 | − |
| chr12 | 111014112 | 111014161 | + | chr20 | 21113122 | 21113434 | + | chr7 | 4399598 | 4399881 | − |
| chr12 | 111081829 | 111082018 | + | chr20 | 21141196 | 21141378 | − | chr7 | 4427163 | 4427450 | + |
| chr12 | 112563364 | 112563827 | + | chr20 | 21149858 | 21150119 | − | chr7 | 5261559 | 5261684 | − |
| chr12 | 112597998 | 112598089 | − | chr20 | 21245682 | 21246041 | + | chr7 | 5579413 | 5579626 | + |
| chr12 | 112643629 | 112643773 | − | chr20 | 21488767 | 21488872 | − | chr7 | 6984053 | 6984378 | − |
| chr12 | 112644300 | 112644507 | − | chr20 | 21489443 | 21489705 | − | chr7 | 6984679 | 6984788 | − |
| chr12 | 112646509 | 112646554 | − | chr20 | 21586494 | 21586810 | − | chr7 | 8762899 | 8763358 | + |
| chr12 | 112672859 | 112673161 | + | chr20 | 21602578 | 21602995 | + | chr7 | 8806702 | 8806796 | + |
| chr12 | 112706109 | 112706171 | + | chr20 | 21603680 | 21603820 | + | chr7 | 8806822 | 8806971 | + |
| chr12 | 112706221 | 112706538 | + | chr20 | 21603803 | 21603964 | + | chr7 | 8811156 | 8811550 | − |
| chr12 | 112719258 | 112719473 | − | chr20 | 21778342 | 21778721 | + | chr7 | 9187343 | 9187534 | + |
| chr12 | 112750231 | 112750584 | − | chr20 | 21795496 | 21795763 | − | chr7 | 9372925 | 9373108 | − |
| chr12 | 112766418 | 112766705 | − | chr20 | 21809300 | 21809425 | − | chr7 | 10238207 | 10238362 | − |
| chr12 | 113322559 | 113322666 | − | chr20 | 21811483 | 21811916 | − | chr7 | 11782950 | 11783072 | − |
| chr12 | 113322980 | 113323168 | − | chr20 | 21821816 | 21822116 | + | chr7 | 11858176 | 11858563 | + |
| chr12 | 113397515 | 113397939 | + | chr20 | 21834630 | 21835074 | − | chr7 | 12284657 | 12284864 | + |
| chr12 | 113433426 | 113433585 | − | chr20 | 21838424 | 21838638 | − | chr7 | 12284898 | 12285039 | + |
| chr12 | 113563162 | 113563387 | + | chr20 | 21840250 | 21840531 | + | chr7 | 13640884 | 13641008 | + |
| chr12 | 113677251 | 113677525 | + | chr20 | 21896294 | 21896586 | + | chr7 | 14088173 | 14088242 | + |
| chr12 | 113940446 | 113940822 | + | chr20 | 21904768 | 21905019 | + | chr7 | 17039577 | 17039964 | + |
| chr12 | 114002050 | 114002177 | − | chr20 | 21909481 | 21909820 | + | chr7 | 18107931 | 18108318 | − |
| chr12 | 114075236 | 114075422 | − | chr20 | 21909824 | 21909904 | + | chr7 | 18305999 | 18306409 | − |
| chr12 | 114204815 | 114205066 | + | chr20 | 21920819 | 21921269 | + | chr7 | 18535948 | 18536416 | + |
| chr12 | 114205103 | 114205164 | + | chr20 | 21940609 | 21941086 | − | chr7 | 18965455 | 18965925 | − |
| chr12 | 114318509 | 114318992 | + | chr20 | 21987056 | 21987278 | + | chr7 | 20325581 | 20325712 | − |
| chr12 | 114344127 | 114344602 | − | chr20 | 22007376 | 22007577 | − | chr7 | 20325930 | 20326065 | − |
| chr12 | 114461000 | 114461413 | + | chr20 | 22033053 | 22033358 | + | chr7 | 22372735 | 22373191 | − |
| chr12 | 114461735 | 114461775 | + | chr20 | 22103334 | 22103820 | + | chr7 | 22418582 | 22418984 | + |
| chr12 | 114528862 | 114529323 | + | chr20 | 22146781 | 22147138 | − | chr7 | 22870303 | 22870685 | + |
| chr12 | 114591903 | 114592090 | − | chr20 | 22233571 | 22233775 | + | chr7 | 24055508 | 24055894 | − |
| chr12 | 114592599 | 114592738 | − | chr20 | 22252530 | 22252942 | − | chr7 | 24227385 | 24227523 | + |
| chr12 | 114602151 | 114602481 | + | chr20 | 22285942 | 22286316 | − | chr7 | 26527345 | 26527439 | + |
| chr12 | 114617242 | 114617323 | − | chr20 | 22292819 | 22292906 | − | chr7 | 26542206 | 26542595 | − |
| chr12 | 114915361 | 114915814 | − | chr20 | 22293284 | 22293617 | − | chr7 | 27341527 | 27341855 | + |
| chr12 | 114957098 | 114957254 | − | chr20 | 22372160 | 22372304 | + | chr7 | 27342069 | 27342249 | + |
| chr12 | 115029236 | 115029518 | − | chr20 | 22438949 | 22439291 | + | chr7 | 27425262 | 27425549 | − |
| chr12 | 115029941 | 115029965 | − | chr20 | 22449027 | 22449418 | + | chr7 | 29852995 | 29853135 | − |
| chr12 | 115261430 | 115261762 | + | chr20 | 22465216 | 22465491 | + | chr7 | 30192119 | 30192454 | + |
| chr12 | 115268958 | 115269186 | − | chr20 | 22493864 | 22494153 | − | chr7 | 30722359 | 30722610 | + |
| chr12 | 115481283 | 115481592 | + | chr20 | 22520289 | 22520517 | − | chr7 | 30722643 | 30722691 | + |
| chr12 | 115625584 | 115625901 | − | chr20 | 22598091 | 22598498 | − | chr7 | 30741747 | 30742186 | + |
| chr12 | 115658085 | 115658555 | − | chr20 | 22649191 | 22649620 | − | chr7 | 31241933 | 31242331 | − |
| chr12 | 115662155 | 115662296 | + | chr20 | 22684815 | 22685087 | − | chr7 | 32306820 | 32306881 | − |
| chr12 | 115663225 | 115663363 | + | chr20 | 22687887 | 22688077 | + | chr7 | 32461596 | 32462083 | − |
| chr12 | 115663849 | 115664012 | + | chr20 | 22689581 | 22689835 | + | chr7 | 33517292 | 33517407 | + |
| chr12 | 115849172 | 115849618 | − | chr20 | 22730491 | 22730962 | + | chr7 | 33517425 | 33517675 | + |
| chr12 | 115852871 | 115852978 | − | chr20 | 22750479 | 22750834 | − | chr7 | 33859717 | 33860133 | − |
| chr12 | 115853325 | 115853542 | − | chr20 | 22770605 | 22770829 | + | chr7 | 33977860 | 33978006 | − |
| chr12 | 117372337 | 117372549 | + | chr20 | 22799009 | 22799478 | − | chr7 | 33978406 | 33978639 | − |
| chr12 | 117372911 | 117373107 | + | chr20 | 22800630 | 22800739 | + | chr7 | 33978985 | 33979072 | − |
| chr12 | 117559831 | 117559953 | + | chr20 | 22837181 | 22837620 | + | chr7 | 35753230 | 35753534 | + |
| chr12 | 117563730 | 117564001 | + | chr20 | 22880697 | 22881040 | − | chr7 | 35912637 | 35912721 | − |
| chr12 | 117748772 | 117749128 | − | chr20 | 22892832 | 22892962 | + | chr7 | 38282092 | 38282218 | + |
| chr12 | 118433664 | 118433862 | − | chr20 | 22899738 | 22900112 | − | chr7 | 38504367 | 38504839 | − |
| chr12 | 118435158 | 118435299 | − | chr20 | 22905924 | 22906340 | + | chr7 | 38677561 | 38677946 | − |
| chr12 | 118512349 | 118512395 | − | chr20 | 22908541 | 22908969 | + | chr7 | 41851704 | 41852141 | + |
| chr12 | 118512690 | 118513076 | − | chr20 | 22920720 | 22920889 | − | chr7 | 42551742 | 42551959 | − |
| chr12 | 118519585 | 118519979 | + | chr20 | 22938687 | 22939069 | − | chr7 | 42723583 | 42723903 | + |
| chr12 | 118520275 | 118520314 | + | chr20 | 23001181 | 23001635 | + | chr7 | 42739008 | 42739094 | − |
| chr12 | 118636194 | 118636529 | + | chr20 | 23074337 | 23074553 | + | chr7 | 42739537 | 42739854 | − |
| chr12 | 118636957 | 118637036 | + | chr20 | 23087237 | 23087435 | − | chr7 | 44542566 | 44542846 | − |
| chr12 | 118723533 | 118723768 | − | chr20 | 23094751 | 23094849 | − | chr7 | 45425489 | 45425750 | + |
| chr12 | 118724238 | 118724402 | − | chr20 | 23166553 | 23166882 | − | chr7 | 45907229 | 45907361 | − |
| chr12 | 118753774 | 118754123 | − | chr20 | 23245761 | 23245935 | + | chr7 | 46790560 | 46790666 | − |
| chr12 | 118788750 | 118788938 | + | chr20 | 23282551 | 23282728 | − | chr7 | 46818061 | 46818370 | − |
| chr12 | 118789146 | 118789386 | + | chr20 | 23333933 | 23333999 | + | chr7 | 46838654 | 46838950 | + |
| chr12 | 118832977 | 118833379 | + | chr20 | 23341521 | 23341811 | − | chr7 | 48694624 | 48695007 | − |
| chr12 | 119083394 | 119083672 | + | chr20 | 23344650 | 23344930 | + | chr7 | 48699650 | 48700095 | − |
| chr12 | 119138428 | 119138742 | + | chr20 | 23449154 | 23449369 | + | chr7 | 48762483 | 48762649 | + |
| chr12 | 119276077 | 119276456 | − | chr20 | 23543136 | 23543486 | + | chr7 | 49277087 | 49277184 | − |
| chr12 | 119289639 | 119289669 | − | chr20 | 23550998 | 23551089 | − | chr7 | 51000891 | 51001202 | + |
| chr12 | 119290040 | 119290094 | − | chr20 | 23671464 | 23671669 | − | chr7 | 51001648 | 51001811 | + |
| chr12 | 119290445 | 119290772 | − | chr20 | 23713424 | 23713623 | − | chr7 | 52378167 | 52378235 | + |
| chr12 | 119328124 | 119328478 | − | chr20 | 23726757 | 23726961 | − | chr7 | 53469851 | 53469945 | + |
| chr12 | 119389042 | 119389246 | + | chr20 | 23727032 | 23727112 | − | chr7 | 54270519 | 54270802 | − |
| chr12 | 119389458 | 119389610 | + | chr20 | 23785489 | 23785688 | − | chr7 | 54576009 | 54576424 | − |
| chr12 | 119661691 | 119661883 | − | chr20 | 23802571 | 23802775 | − | chr7 | 56506117 | 56506227 | + |
| chr12 | 119662511 | 119662731 | − | chr20 | 23802846 | 23802914 | − | chr7 | 57240625 | 57241079 | + |
| chr12 | 119942077 | 119942208 | − | chr20 | 23944955 | 23945163 | − | chr7 | 57862301 | 57862420 | + |
| chr12 | 119942488 | 119942535 | − | chr20 | 23945240 | 23945322 | − | chr7 | 67402199 | 67402543 | + |
| chr12 | 119945783 | 119946004 | + | chr20 | 24019061 | 24019296 | + | chr7 | 67402894 | 67402969 | + |
| chr12 | 119946313 | 119946571 | + | chr20 | 24021924 | 24022020 | + | chr7 | 68240955 | 68241122 | − |
| chr12 | 121026376 | 121026569 | − | chr20 | 24035870 | 24036273 | − | chr7 | 68241516 | 68241616 | − |
| chr12 | 121026869 | 121027081 | − | chr20 | 24049822 | 24050106 | + | chr7 | 68381723 | 68381818 | + |
| chr12 | 121032428 | 121032738 | + | chr20 | 24144436 | 24144729 | + | chr7 | 68382009 | 68382141 | + |
| chr12 | 121128371 | 121128480 | − | chr20 | 24152058 | 24152218 | + | chr7 | 68624619 | 68624984 | + |
| chr12 | 121128755 | 121128917 | − | chr20 | 24255953 | 24256425 | − | chr7 | 68626095 | 68626275 | + |
| chr12 | 121273599 | 121273886 | + | chr20 | 24262179 | 24262492 | + | chr7 | 69553297 | 69553684 | + |
| chr12 | 121755595 | 121756006 | − | chr20 | 24318657 | 24319035 | − | chr7 | 70216422 | 70216877 | + |
| chr12 | 122942966 | 122943392 | + | chr20 | 24320485 | 24320914 | − | chr7 | 71386947 | 71387186 | − |
| chr12 | 124085676 | 124085843 | + | chr20 | 24325657 | 24325961 | − | chr7 | 71387302 | 71387346 | − |
| chr12 | 124121636 | 124121850 | + | chr20 | 24362357 | 24362775 | + | chr7 | 71387621 | 71387753 | − |
| chr12 | 124123011 | 124123250 | + | chr20 | 24410147 | 24410492 | − | chr7 | 71387834 | 71387929 | + |
| chr12 | 124231492 | 124231716 | − | chr20 | 24501824 | 24502249 | + | chr7 | 71390808 | 71390966 | − |
| chr12 | 124662274 | 124662388 | − | chr20 | 24506967 | 24507081 | − | chr7 | 71392140 | 71392417 | − |
| chr12 | 125227095 | 125227179 | + | chr20 | 24533547 | 24533783 | − | chr7 | 73123201 | 73123451 | + |
| chr12 | 125227851 | 125228113 | + | chr20 | 24574080 | 24574162 | + | chr7 | 73124082 | 73124172 | + |
| chr12 | 125962338 | 125962419 | − | chr20 | 24652623 | 24652847 | + | chr7 | 73311739 | 73311890 | + |
| chr12 | 125962791 | 125962850 | − | chr20 | 24652863 | 24653044 | + | chr7 | 73859372 | 73859543 | + |
| chr12 | 125963100 | 125963427 | − | chr20 | 24685321 | 24685400 | + | chr7 | 74423249 | 74423364 | − |
| chr12 | 126395557 | 126395733 | − | chr20 | 24737130 | 24737229 | + | chr7 | 74932022 | 74932272 | + |
| chr12 | 126926224 | 126926486 | − | chr20 | 24737918 | 24738254 | − | chr7 | 74932877 | 74932967 | + |
| chr12 | 127177568 | 127177862 | − | chr20 | 24786326 | 24786404 | + | chr7 | 75268506 | 75268596 | − |
| chr12 | 127209937 | 127210074 | + | chr20 | 24786491 | 24786679 | + | chr7 | 75269229 | 75269388 | − |
| chr12 | 127235924 | 127236151 | + | chr20 | 24786927 | 24787047 | + | chr7 | 75686487 | 75686608 | − |
| chr12 | 127556463 | 127556611 | − | chr20 | 25024565 | 25024667 | + | chr7 | 75764744 | 75764864 | + |
| chr12 | 128700862 | 128701239 | − | chr20 | 25085560 | 25085864 | + | chr7 | 75765166 | 75765420 | + |
| chr12 | 128720043 | 128720245 | + | chr20 | 25091216 | 25091327 | + | chr7 | 75775366 | 75775781 | − |
| chr12 | 129212950 | 129213134 | − | chr20 | 25120505 | 25120873 | − | chr7 | 75801238 | 75801348 | − |
| chr12 | 129213940 | 129214205 | − | chr20 | 25130735 | 25130912 | + | chr7 | 76671113 | 76671283 | + |
| chr12 | 129485042 | 129485487 | − | chr20 | 25147091 | 25147436 | + | chr7 | 76671300 | 76671388 | + |
| chr12 | 129485925 | 129486371 | + | chr20 | 25176236 | 25176293 | + | chr7 | 76671403 | 76671505 | + |
| chr12 | 129570386 | 129570655 | − | chr20 | 25178950 | 25179017 | + | chr7 | 76683047 | 76683368 | − |
| chr12 | 130623620 | 130624070 | + | chr20 | 25190596 | 25190762 | − | chr7 | 76771575 | 76771866 | + |
| chr12 | 131489655 | 131490000 | − | chr20 | 25618660 | 25618940 | + | chr7 | 76942911 | 76943261 | − |
| chr12 | 133050044 | 133050501 | − | chr20 | 25954193 | 25954332 | + | chr7 | 77237095 | 77237561 | − |
| chr12_GL383551v1_alt | 174402 | 174578 | − | chr20 | 29606000 | 29606139 | − | chr7 | 82054327 | 82054648 | + |
| chr12_GL877875v1_alt | 116526 | 116816 | − | chr20 | 30250586 | 30250725 | + | chr7 | 82057990 | 82058328 | + |
| chr13 | 18740734 | 18741039 | − | chr20 | 30612615 | 30612695 | − | chr7 | 82149297 | 82149539 | − |
| chr13 | 18877736 | 18877969 | + | chr20 | 30612686 | 30612992 | − | chr7 | 82150011 | 82150050 | − |
| chr13 | 22288504 | 22288974 | − | chr20 | 31653798 | 31654119 | + | chr7 | 84840503 | 84840833 | + |
| chr13 | 24142986 | 24143105 | − | chr20 | 31890511 | 31890984 | − | chr7 | 85308121 | 85308537 | + |
| chr13 | 26772754 | 26772880 | + | chr20 | 32049729 | 32050060 | + | chr7 | 86830028 | 86830460 | + |
| chr13 | 26773094 | 26773151 | + | chr20 | 32058669 | 32059138 | − | chr7 | 87234674 | 87235112 | + |
| chr13 | 26773540 | 26773697 | + | chr20 | 33151205 | 33151561 | − | chr7 | 88272094 | 88272497 | + |
| chr13 | 28630418 | 28630555 | − | chr20 | 33287214 | 33287371 | − | chr7 | 88332455 | 88332643 | + |
| chr13 | 28630868 | 28631025 | − | chr20 | 33288028 | 33288215 | − | chr7 | 88333192 | 88333336 | + |
| chr13 | 31484265 | 31484345 | − | chr20 | 33311273 | 33311423 | + | chr7 | 89240977 | 89241455 | + |
| chr13 | 32878816 | 32879298 | + | chr20 | 33311644 | 33311787 | + | chr7 | 91561116 | 91561215 | − |
| chr13 | 33820215 | 33820690 | + | chr20 | 33328542 | 33328632 | − | chr7 | 91561567 | 91561910 | − |
| chr13 | 33970540 | 33970994 | − | chr20 | 33404563 | 33404604 | − | chr7 | 93614834 | 93615021 | + |
| chr13 | 34618762 | 34619116 | − | chr20 | 33404898 | 33405081 | − | chr7 | 93756429 | 93756546 | − |
| chr13 | 37140173 | 37140269 | + | chr20 | 33703522 | 33703958 | − | chr7 | 96679877 | 96680334 | + |
| chr13 | 37141138 | 37141487 | + | chr20 | 33929775 | 33930153 | + | chr7 | 97643609 | 97643801 | − |
| chr13 | 37147441 | 37147742 | + | chr20 | 33983012 | 33983377 | − | chr7 | 97646622 | 97646720 | − |
| chr13 | 37148112 | 37148285 | + | chr20 | 35132925 | 35133398 | − | chr7 | 97776799 | 97777223 | − |
| chr13 | 37376098 | 37376399 | + | chr20 | 36277896 | 36278002 | + | chr7 | 98482952 | 98483062 | + |
| chr13 | 37376844 | 37377037 | + | chr20 | 36408146 | 36408509 | − | chr7 | 98483077 | 98483274 | + |
| chr13 | 38477759 | 38478082 | + | chr20 | 36410179 | 36410439 | + | chr7 | 98483659 | 98483750 | + |
| chr13 | 38961120 | 38961400 | + | chr20 | 36410851 | 36410972 | + | chr7 | 98704249 | 98704525 | + |
| chr13 | 39177333 | 39177457 | − | chr20 | 36411289 | 36411342 | + | chr7 | 98705404 | 98705552 | + |
| chr13 | 39177461 | 39177864 | − | chr20 | 36795846 | 36796143 | + | chr7 | 98806227 | 98806365 | + |
| chr13 | 40377675 | 40377859 | + | chr20 | 36943029 | 36943213 | − | chr7 | 98806586 | 98806879 | + |
| chr13 | 41402809 | 41403100 | + | chr20 | 37558810 | 37559255 | + | chr7 | 99265873 | 99266061 | + |
| chr13 | 43779218 | 43779373 | − | chr20 | 37606769 | 37606841 | + | chr7 | 99266184 | 99266356 | + |
| chr13 | 44157042 | 44157418 | − | chr20 | 37610479 | 37610799 | + | chr7 | 99489414 | 99489664 | + |
| chr13 | 44825301 | 44825677 | − | chr20 | 37682582 | 37682952 | − | chr7 | 100286544 | 100286619 | − |
| chr13 | 45168223 | 45168485 | + | chr20 | 37683265 | 37683354 | − | chr7 | 100287212 | 100287462 | − |
| chr13 | 45818030 | 45818330 | + | chr20 | 37932326 | 37932771 | + | chr7 | 101533575 | 101533816 | − |
| chr13 | 45884372 | 45884520 | − | chr20 | 37932957 | 37933130 | − | chr7 | 103932712 | 103932979 | − |
| chr13 | 46756978 | 46757166 | − | chr20 | 37971212 | 37971442 | − | chr7 | 104887266 | 104887715 | − |
| chr13 | 47068963 | 47069361 | − | chr20 | 37971603 | 37971834 | − | chr7 | 106233679 | 106234121 | − |
| chr13 | 47822755 | 47823142 | + | chr20 | 38211910 | 38212032 | + | chr7 | 106852343 | 106852805 | + |
| chr13 | 47826373 | 47826465 | + | chr20 | 38212470 | 38212608 | + | chr7 | 107884435 | 107884738 | + |
| chr13 | 50706929 | 50706991 | − | chr20 | 38316524 | 38316808 | − | chr7 | 107885246 | 107885396 | + |
| chr13 | 50708468 | 50708948 | − | chr20 | 38346838 | 38347049 | − | chr7 | 108350454 | 108350722 | + |
| chr13 | 52338815 | 52339242 | + | chr20 | 38347622 | 38347858 | − | chr7 | 109323213 | 109323605 | − |
| chr13 | 52772175 | 52772628 | + | chr20 | 38351206 | 38351487 | − | chr7 | 109469952 | 109470054 | + |
| chr13 | 52839986 | 52840364 | − | chr20 | 38380173 | 38380228 | − | chr7 | 109997003 | 109997274 | + |
| chr13 | 53419171 | 53419251 | − | chr20 | 38651081 | 38651551 | − | chr7 | 112349892 | 112350308 | − |
| chr13 | 53509862 | 53510229 | − | chr20 | 38655718 | 38656011 | − | chr7 | 113676166 | 113676680 | + |
| chr13 | 55220842 | 55221021 | + | chr20 | 38657068 | 38657222 | − | chr7 | 116036230 | 116036509 | − |
| chr13 | 57875848 | 57875911 | − | chr20 | 38657273 | 38657387 | − | chr7 | 116579838 | 116580184 | − |
| chr13 | 57875942 | 57876227 | − | chr20 | 38657703 | 38657791 | − | chr7 | 118172868 | 118173231 | − |
| chr13 | 58011484 | 58011958 | − | chr20 | 38700138 | 38700251 | + | chr7 | 119466778 | 119466930 | − |
| chr13 | 58360933 | 58361399 | − | chr20 | 38701178 | 38701662 | + | chr7 | 119467287 | 119467465 | − |
| chr13 | 59326865 | 59327246 | + | chr20 | 38735832 | 38736019 | − | chr7 | 121610374 | 121610770 | + |
| chr13 | 59384154 | 59384539 | + | chr20 | 39125137 | 39125314 | + | chr7 | 121764743 | 121765028 | − |
| chr13 | 60290367 | 60290461 | − | chr20 | 39125818 | 39126090 | + | chr7 | 122068820 | 122068863 | − |
| chr13 | 61789571 | 61789912 | − | chr20 | 39138758 | 39138986 | + | chr7 | 122068879 | 122069238 | − |
| chr13 | 62598566 | 62599015 | − | chr20 | 39200783 | 39201265 | − | chr7 | 124114844 | 124115204 | + |
| chr13 | 62981415 | 62981687 | − | chr20 | 39237485 | 39237929 | + | chr7 | 124438176 | 124438423 | + |
| chr13 | 63614617 | 63614826 | − | chr20 | 39293210 | 39293704 | − | chr7 | 125721622 | 125721754 | + |
| chr13 | 69092500 | 69092774 | − | chr20 | 39323372 | 39323580 | + | chr7 | 126338369 | 126338672 | − |
| chr13 | 69615896 | 69616014 | − | chr20 | 39339997 | 39340402 | + | chr7 | 126729185 | 126729553 | + |
| chr13 | 69616063 | 69616287 | − | chr20 | 39358675 | 39358956 | + | chr7 | 127256661 | 127257120 | − |
| chr13 | 69618417 | 69618504 | − | chr20 | 39405869 | 39406150 | + | chr7 | 127513199 | 127513349 | − |
| chr13 | 69619058 | 69619444 | − | chr20 | 39504722 | 39504825 | − | chr7 | 129452508 | 129452553 | + |
| chr13 | 70738297 | 70738485 | + | chr20 | 39505178 | 39505433 | − | chr7 | 129452696 | 129452865 | + |
| chr13 | 70858694 | 70858819 | + | chr20 | 39604890 | 39605131 | − | chr7 | 129452884 | 129453011 | + |
| chr13 | 70932661 | 70932913 | + | chr20 | 39772954 | 39773412 | − | chr7 | 129590976 | 129591318 | − |
| chr13 | 74258801 | 74259226 | − | chr20 | 39830490 | 39830752 | − | chr7 | 130950067 | 130950107 | + |
| chr13 | 74561569 | 74561666 | + | chr20 | 39870207 | 39870700 | − | chr7 | 131008035 | 131008305 | + |
| chr13 | 76286973 | 76287187 | + | chr20 | 40050774 | 40051091 | − | chr7 | 131831884 | 131832068 | − |
| chr13 | 77308621 | 77309065 | − | chr20 | 40240910 | 40241209 | + | chr7 | 134370532 | 134371036 | − |
| chr13 | 77776099 | 77776383 | − | chr20 | 40316973 | 40317298 | + | chr7 | 134615814 | 134616276 | − |
| chr13 | 80567199 | 80567636 | − | chr20 | 40384541 | 40384608 | + | chr7 | 134932443 | 134932901 | − |
| chr13 | 81216450 | 81216916 | − | chr20 | 40384622 | 40384880 | + | chr7 | 134952813 | 134952885 | + |
| chr13 | 81295804 | 81296220 | + | chr20 | 40653548 | 40653986 | − | chr7 | 134964220 | 134964675 | − |
| chr13 | 81881199 | 81881419 | + | chr20 | 40777798 | 40778132 | − | chr7 | 136052664 | 136052741 | + |
| chr13 | 82489676 | 82490029 | + | chr20 | 40805508 | 40805848 | + | chr7 | 136053590 | 136053963 | − |
| chr13 | 83675012 | 83675337 | + | chr20 | 40843514 | 40843798 | + | chr7 | 136103837 | 136104272 | + |
| chr13 | 84902559 | 84903020 | + | chr20 | 40861806 | 40862194 | + | chr7 | 136900522 | 136900930 | − |
| chr13 | 86825723 | 86825802 | + | chr20 | 40949550 | 40949897 | + | chr7 | 139038588 | 139038838 | + |
| chr13 | 87006961 | 87007130 | − | chr20 | 41774694 | 41774818 | − | chr7 | 139519030 | 139519133 | − |
| chr13 | 87323084 | 87323217 | − | chr20 | 41775210 | 41775381 | − | chr7 | 140156443 | 140156868 | − |
| chr13 | 88377902 | 88378358 | − | chr20 | 41775419 | 41775496 | − | chr7 | 141119019 | 141119433 | − |
| chr13 | 88404606 | 88405026 | + | chr20 | 41777975 | 41778284 | − | chr7 | 143170634 | 143170980 | + |
| chr13 | 89038636 | 89039098 | + | chr20 | 41837531 | 41837786 | + | chr7 | 144891693 | 144891936 | + |
| chr13 | 89307320 | 89307701 | − | chr20 | 41856161 | 41856417 | − | chr7 | 144973122 | 144973171 | − |
| chr13 | 89963725 | 89964150 | + | chr20 | 41863237 | 41863343 | − | chr7 | 144973273 | 144973337 | − |
| chr13 | 91088230 | 91088636 | + | chr20 | 41863669 | 41863833 | − | chr7 | 145395378 | 145395829 | − |
| chr13 | 92653036 | 92653456 | − | chr20 | 41874227 | 41874695 | + | chr7 | 146065537 | 146065744 | + |
| chr13 | 95310916 | 95311052 | + | chr20 | 41876912 | 41877381 | + | chr7 | 146066042 | 146066282 | + |
| chr13 | 95312100 | 95312535 | + | chr20 | 41929473 | 41929930 | + | chr7 | 146215892 | 146216309 | − |
| chr13 | 98089356 | 98089804 | − | chr20 | 41940775 | 41941229 | + | chr7 | 146235384 | 146235789 | − |
| chr13 | 99124140 | 99124563 | + | chr20 | 41974380 | 41974848 | − | chr7 | 146615536 | 146615980 | + |
| chr13 | 99192828 | 99193019 | + | chr20 | 42026345 | 42026673 | − | chr7 | 147296672 | 147296862 | − |
| chr13 | 99193026 | 99193085 | + | chr20 | 42035906 | 42036199 | − | chr7 | 147527774 | 147527999 | − |
| chr13 | 99455144 | 99455208 | − | chr20 | 42051304 | 42051427 | − | chr7 | 148276195 | 148276635 | − |
| chr13 | 102408691 | 102408959 | + | chr20 | 42105100 | 42105374 | + | chr7 | 148668918 | 148669068 | + |
| chr13 | 102697695 | 102697953 | − | chr20 | 42136716 | 42137168 | − | chr7 | 149564452 | 149564785 | + |
| chr13 | 103608189 | 103608580 | + | chr20 | 42139331 | 42139680 | − | chr7 | 149565234 | 149565370 | + |
| chr13 | 104303895 | 104304337 | − | chr20 | 42143685 | 42144137 | − | chr7 | 149600078 | 149600218 | + |
| chr13 | 104307606 | 104308059 | − | chr20 | 42355118 | 42355425 | + | chr7 | 150237220 | 150237568 | + |
| chr13 | 104395348 | 104395554 | + | chr20 | 42454883 | 42455040 | + | chr7 | 150237985 | 150238092 | + |
| chr13 | 105169113 | 105169567 | − | chr20 | 42455319 | 42455492 | + | chr7 | 150432811 | 150432977 | − |
| chr13 | 106581877 | 106582108 | + | chr20 | 42645288 | 42645568 | + | chr7 | 150714966 | 150715400 | − |
| chr13 | 107743005 | 107743259 | + | chr20 | 42686290 | 42686457 | + | chr7 | 151382703 | 151382921 | − |
| chr13 | 109379760 | 109379975 | − | chr20 | 42686792 | 42686927 | + | chr7 | 151872413 | 151872776 | − |
| chr13 | 109916139 | 109916397 | − | chr20 | 42748580 | 42749007 | + | chr7 | 152076317 | 152076753 | − |
| chr13 | 110000980 | 110001412 | − | chr20 | 43158999 | 43159268 | − | chr7 | 153731187 | 153731294 | − |
| chr13 | 111912615 | 111912904 | + | chr20 | 43159306 | 43159385 | − | chr7 | 153731710 | 153732058 | − |
| chr13 | 111949570 | 111949758 | + | chr20 | 43211088 | 43211556 | + | chr7 | 155541938 | 155542097 | − |
| chr13 | 112615515 | 112615628 | − | chr20 | 43216471 | 43216889 | − | chr7 | 155567802 | 155567959 | − |
| chr13_KI270840v1_alt | 42384 | 42689 | − | chr20 | 43217432 | 43217919 | − | chr7 | 156135904 | 156136372 | + |
| chr13_KI270840v1_alt | 185276 | 185509 | + | chr20 | 43222585 | 43222994 | − | chr7 | 158399801 | 158399880 | − |
| chr14 | 20021103 | 20021297 | + | chr20 | 43251059 | 43251450 | + | chr7 | 159293171 | 159293546 | − |
| chr14 | 21650546 | 21650709 | − | chr20 | 43251760 | 43251815 | + | chr7_KI270805v1_alt | 77295 | 77374 | + |
| chr14 | 21802081 | 21802188 | + | chr20 | 43281319 | 43281740 | + | chr7_KI270808v1_alt | 61322 | 61762 | − |
| chr14 | 22268884 | 22268975 | + | chr20 | 43317326 | 43317490 | − | chr7_KI270809v1_alt | 157948 | 158118 | + |
| chr14 | 22269716 | 22269816 | + | chr20 | 43318121 | 43318351 | − | chr7_KI270809v1_alt | 158135 | 158223 | + |
| chr14 | 23865894 | 23866222 | + | chr20 | 43339614 | 43339891 | + | chr7_KI270809v1_alt | 158238 | 158340 | + |
| chr14 | 23866415 | 23866544 | + | chr20 | 43343482 | 43343861 | + | chr7_KI270809v1_alt | 169882 | 170203 | − |
| chr14 | 24467463 | 24467941 | − | chr20 | 43344421 | 43344490 | + | chr8 | 322915 | 323351 | − |
| chr14 | 26601713 | 26602030 | − | chr20 | 43350138 | 43350389 | − | chr8 | 2684127 | 2684242 | + |
| chr14 | 26602029 | 26602152 | + | chr20 | 43380022 | 43380150 | + | chr8 | 4878238 | 4878334 | − |
| chr14 | 27536621 | 27536963 | − | chr20 | 43387516 | 43387856 | − | chr8 | 5266741 | 5266988 | − |
| chr14 | 27601278 | 27601509 | − | chr20 | 43438407 | 43438605 | + | chr8 | 5355398 | 5355763 | + |
| chr14 | 28703002 | 28703324 | + | chr20 | 43439063 | 43439350 | + | chr8 | 5998064 | 5998189 | − |
| chr14 | 29066940 | 29067241 | − | chr20 | 43442353 | 43442803 | + | chr8 | 8149280 | 8149389 | + |
| chr14 | 29950607 | 29950879 | − | chr20 | 43736050 | 43736143 | − | chr8 | 8149695 | 8150003 | + |
| chr14 | 29951246 | 29951315 | − | chr20 | 43736682 | 43736835 | − | chr8 | 8930028 | 8930321 | − |
| chr14 | 30452808 | 30453073 | + | chr20 | 43737295 | 43737424 | − | chr8 | 9170282 | 9170618 | + |
| chr14 | 31501746 | 31502197 | − | chr20 | 43869452 | 43869852 | − | chr8 | 9171019 | 9171135 | + |
| chr14 | 31955842 | 31956084 | − | chr20 | 43908809 | 43909190 | + | chr8 | 12515659 | 12515967 | − |
| chr14 | 31956120 | 31956219 | − | chr20 | 44039530 | 44039860 | − | chr8 | 12516273 | 12516382 | − |
| chr14 | 33032499 | 33032850 | + | chr20 | 44055936 | 44056347 | − | chr8 | 13245474 | 13245626 | + |
| chr14 | 35944726 | 35945069 | − | chr20 | 44241769 | 44242227 | + | chr8 | 13823211 | 13823610 | − |
| chr14 | 37844728 | 37845140 | + | chr20 | 44656414 | 44656710 | − | chr8 | 13967755 | 13968078 | + |
| chr14 | 37933187 | 37933444 | − | chr20 | 44761530 | 44761768 | + | chr8 | 15677919 | 15678379 | − |
| chr14 | 37970770 | 37971078 | + | chr20 | 44766752 | 44767212 | + | chr8 | 16329488 | 16329613 | + |
| chr14 | 38032733 | 38033120 | + | chr20 | 44796977 | 44797174 | + | chr8 | 16330637 | 16330993 | + |
| chr14 | 38272618 | 38272984 | − | chr20 | 44797397 | 44797530 | + | chr8 | 17486392 | 17486691 | + |
| chr14 | 40661230 | 40661586 | − | chr20 | 44797622 | 44797679 | + | chr8 | 17487355 | 17487717 | + |
| chr14 | 41158906 | 41159127 | − | chr20 | 45450060 | 45450386 | − | chr8 | 18208897 | 18209114 | − |
| chr14 | 41271918 | 41272090 | − | chr20 | 45540319 | 45540509 | + | chr8 | 18358759 | 18359075 | − |
| chr14 | 41425977 | 41426017 | + | chr20 | 45541426 | 45541634 | + | chr8 | 18389836 | 18390117 | − |
| chr14 | 41427054 | 41427350 | + | chr20 | 46108551 | 46109002 | − | chr8 | 20565604 | 20565758 | − |
| chr14 | 42188712 | 42188819 | + | chr20 | 46132091 | 46132475 | + | chr8 | 20566139 | 20566241 | − |
| chr14 | 42193075 | 42193173 | + | chr20 | 46325737 | 46326109 | − | chr8 | 20972849 | 20972945 | − |
| chr14 | 42194247 | 42194546 | + | chr20 | 46538081 | 46538264 | + | chr8 | 21602749 | 21603147 | + |
| chr14 | 42333413 | 42333775 | − | chr20 | 46539117 | 46539381 | + | chr8 | 21609498 | 21609595 | + |
| chr14 | 44637165 | 44637581 | + | chr20 | 46614033 | 46614226 | − | chr8 | 21819778 | 21820224 | − |
| chr14 | 46672242 | 46672600 | + | chr20 | 46614221 | 46614548 | − | chr8 | 24027790 | 24028204 | − |
| chr14 | 46829361 | 46829602 | − | chr20 | 46618927 | 46619135 | + | chr8 | 27394137 | 27394286 | + |
| chr14 | 47102508 | 47102835 | − | chr20 | 46696729 | 46697058 | − | chr8 | 27395491 | 27395871 | + |
| chr14 | 47586388 | 47586694 | − | chr20 | 46792871 | 46793344 | − | chr8 | 28847692 | 28847938 | − |
| chr14 | 47693040 | 47693314 | + | chr20 | 46815658 | 46816076 | − | chr8 | 28847957 | 28848068 | − |
| chr14 | 47758199 | 47758492 | + | chr20 | 46816142 | 46816445 | − | chr8 | 29965443 | 29965886 | − |
| chr14 | 48093180 | 48093658 | − | chr20 | 46950767 | 46951149 | − | chr8 | 30360984 | 30361357 | + |
| chr14 | 48744581 | 48744741 | + | chr20 | 46962764 | 46963255 | + | chr8 | 30958097 | 30958192 | − |
| chr14 | 49167411 | 49167844 | + | chr20 | 47073664 | 47074039 | + | chr8 | 32898463 | 32898560 | + |
| chr14 | 50423773 | 50424148 | + | chr20 | 47098963 | 47099272 | − | chr8 | 35201876 | 35202143 | + |
| chr14 | 51192099 | 51192443 | − | chr20 | 47099519 | 47099658 | − | chr8 | 35897220 | 35897523 | + |
| chr14 | 51526245 | 51526647 | + | chr20 | 47116553 | 47116813 | + | chr8 | 35897736 | 35898082 | + |
| chr14 | 52355616 | 52356054 | + | chr20 | 47799097 | 47799551 | − | chr8 | 36235700 | 36235834 | − |
| chr14 | 55749162 | 55749581 | − | chr20 | 47925261 | 47925717 | + | chr8 | 36236024 | 36236323 | − |
| chr14 | 55804437 | 55804727 | + | chr20 | 47942584 | 47942712 | − | chr8 | 36538691 | 36539124 | + |
| chr14 | 57463729 | 57464144 | + | chr20 | 47983590 | 47983743 | + | chr8 | 37335125 | 37335560 | − |
| chr14 | 57502390 | 57502831 | − | chr20 | 48196975 | 48197413 | + | chr8 | 37424897 | 37425193 | + |
| chr14 | 58022498 | 58022724 | + | chr20 | 48210260 | 48210570 | + | chr8 | 37425518 | 37425749 | + |
| chr14 | 58886227 | 58886673 | + | chr20 | 48359262 | 48359408 | − | chr8 | 40618820 | 40619283 | − |
| chr14 | 59075597 | 59075945 | + | chr20 | 48413072 | 48413169 | + | chr8 | 40937354 | 40937680 | − |
| chr14 | 61434998 | 61435320 | + | chr20 | 48413233 | 48413280 | + | chr8 | 40948511 | 40948578 | − |
| chr14 | 61614357 | 61614776 | + | chr20 | 48430991 | 48431108 | − | chr8 | 40948772 | 40949176 | − |
| chr14 | 64566761 | 64567098 | + | chr20 | 48431894 | 48432052 | − | chr8 | 41138262 | 41138504 | − |
| chr14 | 65227548 | 65227665 | + | chr20 | 48458595 | 48459130 | − | chr8 | 48846850 | 48847144 | + |
| chr14 | 65312607 | 65312979 | − | chr20 | 48528136 | 48528380 | + | chr8 | 49212202 | 49212635 | − |
| chr14 | 66364443 | 66364665 | + | chr20 | 48552453 | 48552580 | + | chr8 | 51984732 | 51984913 | + |
| chr14 | 68775469 | 68775582 | + | chr20 | 48552635 | 48552957 | + | chr8 | 52282446 | 52282542 | − |
| chr14 | 68775606 | 68775884 | + | chr20 | 48553736 | 48553775 | + | chr8 | 52282577 | 52282662 | − |
| chr14 | 68854818 | 68854894 | − | chr20 | 48752452 | 48752823 | + | chr8 | 52782716 | 52782916 | + |
| chr14 | 68855402 | 68855645 | − | chr20 | 48758012 | 48758183 | − | chr8 | 52783636 | 52783684 | + |
| chr14 | 68864418 | 68864868 | − | chr20 | 48758263 | 48758415 | − | chr8 | 55310752 | 55311213 | − |
| chr14 | 69866977 | 69867391 | − | chr20 | 48911986 | 48912183 | − | chr8 | 55341106 | 55341408 | − |
| chr14 | 73836230 | 73836277 | − | chr20 | 48912350 | 48912799 | + | chr8 | 59942784 | 59943093 | − |
| chr14 | 73836568 | 73836657 | − | chr20 | 49474813 | 49475143 | − | chr8 | 61834064 | 61834208 | − |
| chr14 | 74404516 | 74404723 | − | chr20 | 49600543 | 49600796 | + | chr8 | 62230076 | 62230500 | + |
| chr14 | 74649295 | 74649720 | + | chr20 | 49769042 | 49769261 | − | chr8 | 62766545 | 62766905 | − |
| chr14 | 75344361 | 75344843 | + | chr20 | 49769602 | 49769838 | − | chr8 | 64471870 | 64472210 | − |
| chr14 | 76479359 | 76479426 | + | chr20 | 49789671 | 49790144 | + | chr8 | 65041753 | 65042229 | + |
| chr14 | 76527624 | 76528046 | + | chr20 | 50275930 | 50276139 | + | chr8 | 65213420 | 65213794 | + |
| chr14 | 76566215 | 76566539 | − | chr20 | 50317621 | 50318058 | − | chr8 | 67399678 | 67399902 | + |
| chr14 | 76651760 | 76652198 | + | chr20 | 50332261 | 50332594 | − | chr8 | 67401257 | 67401495 | + |
| chr14 | 77237350 | 77237701 | − | chr20 | 50431312 | 50431750 | + | chr8 | 69753098 | 69753506 | − |
| chr14 | 77487675 | 77487858 | − | chr20 | 50482317 | 50482537 | − | chr8 | 69788884 | 69788941 | − |
| chr14 | 77871092 | 77871355 | − | chr20 | 50482956 | 50483133 | − | chr8 | 71915322 | 71915688 | − |
| chr14 | 78113610 | 78113943 | + | chr20 | 50483434 | 50483688 | − | chr8 | 72903153 | 72903413 | − |
| chr14 | 78536131 | 78536222 | + | chr20 | 50494925 | 50495032 | − | chr8 | 73083936 | 73084107 | − |
| chr14 | 78536347 | 78536429 | + | chr20 | 50544636 | 50544748 | − | chr8 | 74152273 | 74152566 | + |
| chr14 | 78927984 | 78928233 | + | chr20 | 51017034 | 51017096 | + | chr8 | 74152773 | 74152884 | + |
| chr14 | 79438180 | 79438288 | + | chr20 | 52223950 | 52224162 | − | chr8 | 74491097 | 74491503 | + |
| chr14 | 82022540 | 82022928 | − | chr20 | 52225046 | 52225216 | − | chr8 | 74715955 | 74716204 | − |
| chr14 | 82688267 | 82688525 | + | chr20 | 52240781 | 52241174 | − | chr8 | 75657447 | 75657851 | − |
| chr14 | 83597274 | 83597348 | + | chr20 | 52274953 | 52275236 | + | chr8 | 76152529 | 76152747 | + |
| chr14 | 83846437 | 83846796 | + | chr20 | 52294197 | 52294623 | − | chr8 | 78281161 | 78281273 | + |
| chr14 | 83971888 | 83972109 | + | chr20 | 52308318 | 52308667 | + | chr8 | 78571349 | 78571783 | + |
| chr14 | 83972557 | 83972616 | + | chr20 | 52330751 | 52331234 | + | chr8 | 79054163 | 79054381 | − |
| chr14 | 84220432 | 84220495 | + | chr20 | 52337650 | 52338135 | − | chr8 | 79054399 | 79054608 | − |
| chr14 | 84220732 | 84221099 | + | chr20 | 52396363 | 52396807 | + | chr8 | 81156681 | 81156920 | + |
| chr14 | 85252148 | 85252372 | − | chr20 | 52502962 | 52503084 | − | chr8 | 81156943 | 81157063 | + |
| chr14 | 85375153 | 85375259 | − | chr20 | 52510098 | 52510501 | + | chr8 | 81157257 | 81157378 | + |
| chr14 | 85421519 | 85421971 | + | chr20 | 52513947 | 52514213 | + | chr8 | 81260201 | 81260646 | + |
| chr14 | 86532290 | 86532749 | − | chr20 | 52570781 | 52571234 | − | chr8 | 81378724 | 81379043 | + |
| chr14 | 86771739 | 86771973 | − | chr20 | 52582173 | 52582507 | + | chr8 | 81955476 | 81955558 | + |
| chr14 | 87532179 | 87532353 | + | chr20 | 52590793 | 52591226 | − | chr8 | 82406055 | 82406408 | − |
| chr14 | 87727173 | 87727522 | − | chr20 | 52647148 | 52647480 | − | chr8 | 82672897 | 82673029 | + |
| chr14 | 88048538 | 88048703 | + | chr20 | 52962556 | 52962861 | + | chr8 | 82673043 | 82673113 | + |
| chr14 | 88305658 | 88306131 | − | chr20 | 53145392 | 53145741 | − | chr8 | 82741867 | 82742248 | − |
| chr14 | 88361286 | 88361754 | + | chr20 | 53362949 | 53363327 | + | chr8 | 83122893 | 83123242 | − |
| chr14 | 89138365 | 89138570 | − | chr20 | 53412995 | 53413376 | − | chr8 | 86670720 | 86670879 | + |
| chr14 | 89138734 | 89138860 | − | chr20 | 53497212 | 53497701 | − | chr8 | 87564841 | 87565275 | + |
| chr14 | 89610167 | 89610566 | + | chr20 | 54382586 | 54383020 | − | chr8 | 89216532 | 89216684 | + |
| chr14 | 89669200 | 89669343 | − | chr20 | 54729332 | 54729659 | + | chr8 | 90124491 | 90124803 | − |
| chr14 | 89669394 | 89669472 | − | chr20 | 54940264 | 54940430 | − | chr8 | 90165936 | 90166131 | + |
| chr14 | 90065386 | 90065840 | − | chr20 | 54940626 | 54940750 | − | chr8 | 90166779 | 90166872 | + |
| chr14 | 92202975 | 92203313 | − | chr20 | 57127739 | 57127789 | + | chr8 | 90221370 | 90221562 | − |
| chr14 | 92214313 | 92214591 | + | chr20 | 57127851 | 57128085 | + | chr8 | 90221581 | 90221792 | − |
| chr14 | 92255760 | 92256203 | + | chr20 | 57143161 | 57143356 | + | chr8 | 90543049 | 90543390 | − |
| chr14 | 92558529 | 92558870 | − | chr20 | 57216278 | 57216460 | − | chr8 | 91781871 | 91782050 | − |
| chr14 | 92559167 | 92559318 | − | chr20 | 57411614 | 57411795 | + | chr8 | 91782429 | 91782685 | − |
| chr14 | 92919639 | 92919788 | − | chr20 | 57585184 | 57585578 | + | chr8 | 92613207 | 92613669 | − |
| chr14 | 92920395 | 92920567 | − | chr20 | 57627311 | 57627764 | − | chr8 | 92860241 | 92860317 | + |
| chr14 | 93764877 | 93765169 | + | chr20 | 57783578 | 57783789 | + | chr8 | 93876865 | 93877228 | − |
| chr14 | 94123231 | 94123571 | − | chr20 | 57806951 | 57807391 | + | chr8 | 94289171 | 94289348 | − |
| chr14 | 94875647 | 94875731 | + | chr20 | 57811102 | 57811376 | + | chr8 | 95350288 | 95350469 | − |
| chr14 | 94876044 | 94876221 | + | chr20 | 57945444 | 57945678 | + | chr8 | 95350680 | 95350743 | − |
| chr14 | 94901058 | 94901507 | + | chr20 | 57970394 | 57970628 | + | chr8 | 95350762 | 95350859 | − |
| chr14 | 94981135 | 94981515 | − | chr20 | 58099752 | 58099935 | + | chr8 | 95835217 | 95835392 | + |
| chr14 | 95063350 | 95063814 | − | chr20 | 58178653 | 58178912 | − | chr8 | 97103614 | 97103849 | − |
| chr14 | 97818691 | 97819000 | − | chr20 | 58586113 | 58586568 | + | chr8 | 97103855 | 97103935 | − |
| chr14 | 98027681 | 98028025 | + | chr20 | 59186096 | 59186281 | + | chr8 | 97188713 | 97188850 | + |
| chr14 | 98460789 | 98460990 | + | chr20 | 59389667 | 59389940 | − | chr8 | 97188869 | 97189051 | + |
| chr14 | 98690868 | 98691240 | − | chr20 | 59433568 | 59433747 | − | chr8 | 97190151 | 97190194 | + |
| chr14 | 98821379 | 98821654 | + | chr20 | 59441536 | 59441926 | − | chr8 | 97470367 | 97470769 | + |
| chr14 | 98884681 | 98884854 | − | chr20 | 60060014 | 60060129 | − | chr8 | 97580588 | 97580835 | − |
| chr14 | 99369852 | 99370011 | − | chr20 | 60242974 | 60243230 | + | chr8 | 98336489 | 98336975 | − |
| chr14 | 99370322 | 99370566 | − | chr20 | 60252835 | 60253073 | − | chr8 | 98403323 | 98403715 | − |
| chr14 | 99541444 | 99541577 | − | chr20 | 60481135 | 60481246 | − | chr8 | 98403822 | 98403868 | − |
| chr14 | 99689239 | 99689473 | − | chr20 | 60928092 | 60928186 | + | chr8 | 100861769 | 100861898 | − |
| chr14 | 100607634 | 100608022 | + | chr20 | 60938015 | 60938152 | + | chr8 | 101014159 | 101014227 | − |
| chr14 | 100672644 | 100672803 | + | chr20 | 61105355 | 61105769 | − | chr8 | 101014692 | 101014791 | − |
| chr14 | 100768354 | 100768543 | + | chr20 | 61794222 | 61794643 | − | chr8 | 101285010 | 101285282 | + |
| chr14 | 101176887 | 101177312 | + | chr20 | 63114306 | 63114558 | − | chr8 | 102161947 | 102162388 | + |
| chr14 | 101407564 | 101407695 | − | chr20 | 63270957 | 63271056 | + | chr8 | 102417206 | 102417507 | − |
| chr14 | 101516336 | 101516478 | − | chr20_GL383577v2_alt | 20 | 296 | + | chr8 | 102716989 | 102717082 | + |
| chr14 | 101687935 | 101688267 | + | chr20_GL383577v2_alt | 4898 | 5155 | + | chr8 | 102717366 | 102717481 | + |
| chr14 | 102626353 | 102626463 | − | chr20_GL383577v2_alt | 5967 | 6063 | + | chr8 | 102717944 | 102718205 | + |
| chr14 | 104212073 | 104212359 | − | chr20_GL383577v2_alt | 26658 | 26703 | − | chr8 | 103235455 | 103235592 | + |
| chr14 | 104845996 | 104846233 | − | chr20_GL383577v2_alt | 27073 | 27529 | + | chr8 | 103266645 | 103266981 | − |
| chr14 | 105804396 | 105804650 | + | chr20_GL383577v2_alt | 39049 | 39149 | + | chr8 | 103744725 | 103744968 | + |
| chr14 | 105805946 | 105806397 | + | chr20_GL383577v2_alt | 39331 | 39412 | + | chr8 | 103767830 | 103768103 | − |
| chr14_KI270722v1_random | 154425 | 154652 | + | chr20_GL383577v2_alt | 70177 | 70422 | − | chr8 | 104458034 | 104458154 | − |
| chr14_KI270725v1_random | 88165 | 88417 | + | chr20_GL383577v2_alt | 96450 | 96833 | − | chr8 | 104777820 | 104777892 | − |
| chr14_KI270846v1_alt | 272165 | 272419 | + | chr20_GL383577v2_alt | 117901 | 118053 | + | chr8 | 104778258 | 104778655 | − |
| chr14_KI270846v1_alt | 273715 | 274166 | + | chr20_GL383577v2_alt | 125669 | 125760 | − | chr8 | 104879600 | 104880068 | + |
| chr14_KI270847v1_alt | 34201 | 34350 | − | chr21 | 13845764 | 13846089 | − | chr8 | 105567256 | 105567633 | + |
| chr14_KI270847v1_alt | 34957 | 35129 | − | chr21 | 14677987 | 14678439 | − | chr8 | 106043822 | 106044300 | + |
| chr14_KI270847v1_alt | 879439 | 879731 | + | chr21 | 16201714 | 16201964 | − | chr8 | 106831785 | 106832113 | + |
| chr14_KI270847v1_alt | 1237793 | 1238133 | − | chr21 | 17494792 | 17495236 | + | chr8 | 108674864 | 108675307 | + |
| chr15 | 20981931 | 20982363 | − | chr21 | 17856350 | 17856630 | + | chr8 | 109794687 | 109794945 | − |
| chr15 | 21555670 | 21556102 | − | chr21 | 18013126 | 18013294 | + | chr8 | 109794968 | 109795058 | − |
| chr15 | 21992963 | 21993395 | − | chr21 | 18406034 | 18406358 | − | chr8 | 109841704 | 109842066 | + |
| chr15 | 25447574 | 25447804 | − | chr21 | 18578270 | 18578544 | + | chr8 | 110641014 | 110641189 | − |
| chr15 | 25447837 | 25447976 | − | chr21 | 18579458 | 18579814 | + | chr8 | 110911634 | 110911776 | − |
| chr15 | 25463381 | 25463710 | + | chr21 | 18623652 | 18624116 | − | chr8 | 111313834 | 111313978 | − |
| chr15 | 25613951 | 25614143 | + | chr21 | 20837059 | 20837449 | − | chr8 | 114183571 | 114183922 | + |
| chr15 | 26685114 | 26685280 | + | chr21 | 20868725 | 20868799 | + | chr8 | 114607815 | 114608098 | + |
| chr15 | 26807914 | 26808174 | + | chr21 | 21815656 | 21815925 | − | chr8 | 114752481 | 114752639 | − |
| chr15 | 26843090 | 26843314 | + | chr21 | 22131834 | 22132039 | − | chr8 | 114846438 | 114846882 | − |
| chr15 | 27374572 | 27374822 | − | chr21 | 22321165 | 22321321 | − | chr8 | 116095359 | 116095537 | − |
| chr15 | 32117054 | 32117355 | − | chr21 | 22641928 | 22642108 | + | chr8 | 116535101 | 116535527 | − |
| chr15 | 33301152 | 33301473 | + | chr21 | 22675363 | 22675686 | + | chr8 | 117313952 | 117314250 | − |
| chr15 | 33392341 | 33392780 | − | chr21 | 22804211 | 22804467 | + | chr8 | 117727228 | 117727432 | + |
| chr15 | 34465752 | 34466171 | − | chr21 | 23065317 | 23065500 | + | chr8 | 120799150 | 120799279 | + |
| chr15 | 35632379 | 35632574 | − | chr21 | 23441702 | 23441928 | + | chr8 | 120993421 | 120993892 | − |
| chr15 | 35685283 | 35685342 | − | chr21 | 23442449 | 23442669 | − | chr8 | 121493669 | 121494076 | − |
| chr15 | 35685345 | 35685616 | − | chr21 | 23982894 | 23983213 | + | chr8 | 121964691 | 121965091 | − |
| chr15 | 35849592 | 35850028 | − | chr21 | 24324223 | 24324685 | − | chr8 | 122007565 | 122007683 | − |
| chr15 | 36062553 | 36062988 | + | chr21 | 24727699 | 24728098 | + | chr8 | 122008084 | 122008341 | − |
| chr15 | 36471100 | 36471422 | − | chr21 | 27009095 | 27009362 | + | chr8 | 122113290 | 122113686 | + |
| chr15 | 37705548 | 37706029 | + | chr21 | 27152819 | 27152948 | − | chr8 | 122236962 | 122237407 | − |
| chr15 | 38547217 | 38547626 | + | chr21 | 28711326 | 28711466 | + | chr8 | 122444648 | 122444908 | − |
| chr15 | 38650825 | 38650941 | − | chr21 | 28712099 | 28712322 | + | chr8 | 122531389 | 122531647 | − |
| chr15 | 39414890 | 39415157 | − | chr21 | 28725279 | 28725481 | − | chr8 | 122531686 | 122531759 | − |
| chr15 | 43237175 | 43237450 | + | chr21 | 28763820 | 28764203 | + | chr8 | 122567206 | 122567496 | − |
| chr15 | 43237786 | 43237952 | + | chr21 | 29950088 | 29950194 | + | chr8 | 122657798 | 122658085 | − |
| chr15 | 45782792 | 45783031 | + | chr21 | 29950404 | 29950623 | + | chr8 | 122725726 | 122725968 | − |
| chr15 | 45936075 | 45936491 | + | chr21 | 29958538 | 29958884 | − | chr8 | 122726416 | 122726561 | − |
| chr15 | 45961734 | 45961891 | − | chr21 | 31043994 | 31044050 | + | chr8 | 122741128 | 122741329 | − |
| chr15 | 45962182 | 45962514 | − | chr21 | 31044724 | 31045135 | + | chr8 | 122805069 | 122805527 | − |
| chr15 | 45984156 | 45984402 | + | chr21 | 33146477 | 33146880 | + | chr8 | 123181574 | 123181904 | + |
| chr15 | 45995218 | 45995439 | + | chr21 | 34635350 | 34635757 | − | chr8 | 123285601 | 123286028 | − |
| chr15 | 45996358 | 45996541 | + | chr21 | 35167391 | 35167848 | − | chr8 | 123461105 | 123461436 | + |
| chr15 | 45996570 | 45996624 | − | chr21 | 35677542 | 35677908 | + | chr8 | 125777585 | 125777979 | + |
| chr15 | 46170851 | 46171137 | + | chr21 | 36136469 | 36136608 | + | chr8 | 125870866 | 125871175 | − |
| chr15 | 46174615 | 46174976 | + | chr21 | 36734927 | 36735038 | − | chr8 | 125940032 | 125940319 | + |
| chr15 | 46235801 | 46236051 | − | chr21 | 36735112 | 36735287 | − | chr8 | 126329785 | 126330200 | − |
| chr15 | 46305389 | 46305613 | + | chr21 | 38037249 | 38037376 | + | chr8 | 126647473 | 126647910 | + |
| chr15 | 46849770 | 46849924 | − | chr21 | 38037442 | 38037594 | + | chr8 | 126882345 | 126882814 | + |
| chr15 | 46917718 | 46917800 | + | chr21 | 38338392 | 38338589 | − | chr8 | 126936060 | 126936509 | + |
| chr15 | 47834864 | 47835179 | + | chr21 | 38717193 | 38717334 | − | chr8 | 127019133 | 127019429 | + |
| chr15 | 48609364 | 48609752 | − | chr21 | 38717809 | 38718073 | − | chr8 | 127239626 | 127239956 | + |
| chr15 | 49289521 | 49289844 | + | chr21 | 38727862 | 38728240 | + | chr8 | 127240246 | 127240365 | + |
| chr15 | 51105849 | 51105935 | + | chr21 | 39939741 | 39940080 | + | chr8 | 127676183 | 127676570 | + |
| chr15 | 51135522 | 51135899 | + | chr21 | 40754340 | 40754758 | − | chr8 | 128365856 | 128366267 | + |
| chr15 | 51186246 | 51186712 | − | chr21 | 41132470 | 41132821 | − | chr8 | 129330106 | 129330497 | − |
| chr15 | 51557986 | 51558088 | + | chr21 | 41135792 | 41135997 | + | chr8 | 129618426 | 129618498 | − |
| chr15 | 51558259 | 51558372 | + | chr21 | 41136010 | 41136196 | + | chr8 | 129618675 | 129618926 | − |
| chr15 | 51710914 | 51711193 | − | chr21 | 41179819 | 41179883 | − | chr8 | 130380093 | 130380508 | − |
| chr15 | 52725082 | 52725277 | + | chr21 | 41187100 | 41187293 | − | chr8 | 130407219 | 130407346 | − |
| chr15 | 52725789 | 52726021 | + | chr21 | 41212490 | 41212738 | − | chr8 | 130411281 | 130411612 | + |
| chr15 | 52861803 | 52862028 | + | chr21 | 42000262 | 42000420 | + | chr8 | 130627566 | 130627830 | + |
| chr15 | 52980883 | 52981336 | + | chr21 | 42707532 | 42707671 | + | chr8 | 130628513 | 130628655 | + |
| chr15 | 53863729 | 53864185 | + | chr21 | 45020574 | 45020668 | + | chr8 | 130802687 | 130803133 | + |
| chr15 | 54040174 | 54040348 | − | chr21_GL383579v2_alt | 29361 | 29566 | − | chr8 | 132022790 | 132022958 | + |
| chr15 | 54040516 | 54040721 | − | chr22 | 10687828 | 10688097 | + | chr8 | 132023078 | 132023327 | + |
| chr15 | 54571682 | 54572113 | + | chr22 | 16906382 | 16906634 | + | chr8 | 132466523 | 132466689 | + |
| chr15 | 55010123 | 55010552 | + | chr22 | 16932426 | 16932834 | − | chr8 | 132466935 | 132466998 | + |
| chr15 | 55113173 | 55113267 | − | chr22 | 21648989 | 21649193 | − | chr8 | 132602304 | 132602555 | + |
| chr15 | 55113668 | 55113821 | − | chr22 | 21649461 | 21649712 | − | chr8 | 132954337 | 132954483 | − |
| chr15 | 55113867 | 55113985 | − | chr22 | 24870567 | 24871020 | + | chr8 | 133157771 | 133158070 | + |
| chr15 | 56326527 | 56326764 | − | chr22 | 24994266 | 24994337 | − | chr8 | 133416048 | 133416464 | + |
| chr15 | 56800935 | 56801368 | + | chr22 | 24994350 | 24994484 | − | chr8 | 133772610 | 133772702 | − |
| chr15 | 57663351 | 57663717 | − | chr22 | 25002265 | 25002395 | − | chr8 | 133773286 | 133773497 | − |
| chr15 | 59852616 | 59852827 | − | chr22 | 25002411 | 25002533 | − | chr8 | 133818132 | 133818271 | + |
| chr15 | 59853194 | 59853360 | − | chr22 | 25042773 | 25042837 | − | chr8 | 133818636 | 133818943 | + |
| chr15 | 60184319 | 60184589 | − | chr22 | 25212609 | 25212867 | + | chr8 | 134108024 | 134108146 | − |
| chr15 | 61274505 | 61274702 | − | chr22 | 25213702 | 25213870 | + | chr8 | 134108177 | 134108401 | − |
| chr15 | 61518583 | 61519017 | + | chr22 | 25800445 | 25800691 | − | chr8 | 134294635 | 134295019 | − |
| chr15 | 61821015 | 61821312 | + | chr22 | 26067339 | 26067811 | + | chr8 | 134305175 | 134305259 | − |
| chr15 | 63111304 | 63111687 | − | chr22 | 26083281 | 26083555 | − | chr8 | 134323544 | 134323678 | + |
| chr15 | 63427551 | 63427677 | − | chr22 | 26155127 | 26155562 | + | chr8 | 134323973 | 134324207 | + |
| chr15 | 63428092 | 63428430 | − | chr22 | 26306914 | 26307234 | − | chr8 | 134439109 | 134439311 | + |
| chr15 | 65999904 | 65999937 | − | chr22 | 26336195 | 26336508 | + | chr8 | 134739808 | 134740177 | + |
| chr15 | 66000010 | 66000132 | − | chr22 | 26388388 | 26388732 | − | chr8 | 134941489 | 134941919 | − |
| chr15 | 66123381 | 66123769 | − | chr22 | 26402791 | 26403101 | + | chr8 | 135054067 | 135054532 | + |
| chr15 | 66750574 | 66750966 | − | chr22 | 26408161 | 26408483 | − | chr8 | 135332764 | 135333158 | + |
| chr15 | 66881970 | 66882371 | − | chr22 | 26649560 | 26649632 | + | chr8 | 135361319 | 135361714 | + |
| chr15 | 67039758 | 67040191 | − | chr22 | 26649922 | 26650034 | + | chr8 | 135391490 | 135391662 | + |
| chr15 | 68440223 | 68440544 | + | chr22 | 26650395 | 26650557 | + | chr8 | 135670178 | 135670484 | + |
| chr15 | 69646653 | 69646797 | + | chr22 | 26704551 | 26704822 | + | chr8 | 135806017 | 135806290 | − |
| chr15 | 70180792 | 70181244 | − | chr22 | 26711102 | 26711565 | − | chr8 | 136066749 | 136066837 | + |
| chr15 | 70632102 | 70632309 | + | chr22 | 26891321 | 26891617 | − | chr8 | 136067097 | 136067450 | + |
| chr15 | 70632726 | 70632880 | + | chr22 | 26946620 | 26947071 | + | chr8 | 136217066 | 136217529 | + |
| chr15 | 71092195 | 71092521 | + | chr22 | 26962233 | 26962619 | + | chr8 | 136274326 | 136274787 | + |
| chr15 | 71567240 | 71567575 | − | chr22 | 27112889 | 27113165 | + | chr8 | 136592208 | 136592490 | − |
| chr15 | 72013062 | 72013498 | − | chr22 | 27113214 | 27113257 | + | chr8 | 136716414 | 136716683 | − |
| chr15 | 72423206 | 72423576 | − | chr22 | 27119986 | 27120436 | + | chr8 | 136837959 | 136838431 | + |
| chr15 | 72918481 | 72918796 | + | chr22 | 27198479 | 27198857 | − | chr8 | 136985501 | 136985599 | + |
| chr15 | 73438562 | 73438862 | − | chr22 | 27216655 | 27216920 | + | chr8 | 136985670 | 136986022 | + |
| chr15 | 73903169 | 73903628 | + | chr22 | 27308618 | 27308955 | + | chr8 | 137842365 | 137842593 | − |
| chr15 | 74991781 | 74992195 | + | chr22 | 27438883 | 27439224 | − | chr8 | 137842764 | 137842963 | − |
| chr15 | 75832632 | 75832943 | + | chr22 | 27458863 | 27459318 | + | chr8 | 137921622 | 137921877 | + |
| chr15 | 76401728 | 76401873 | − | chr22 | 27476089 | 27476310 | − | chr8 | 137921894 | 137922031 | + |
| chr15 | 76402171 | 76402257 | − | chr22 | 27509105 | 27509570 | − | chr8 | 138673798 | 138674169 | + |
| chr15 | 77584160 | 77584629 | + | chr22 | 27693064 | 27693342 | + | chr8 | 138689351 | 138689448 | − |
| chr15 | 78644850 | 78644930 | + | chr22 | 27834798 | 27835133 | − | chr8 | 138689741 | 138689869 | − |
| chr15 | 78645494 | 78645753 | + | chr22 | 30371018 | 30371286 | − | chr8 | 138817112 | 138817560 | − |
| chr15 | 79123897 | 79124138 | − | chr22 | 30547405 | 30547828 | − | chr8 | 138842886 | 138843285 | − |
| chr15 | 79129060 | 79129481 | − | chr22 | 31049103 | 31049451 | + | chr8 | 139056328 | 139056660 | + |
| chr15 | 79129917 | 79130041 | − | chr22 | 32728545 | 32728936 | − | chr8 | 139057155 | 139057219 | + |
| chr15 | 79130319 | 79130677 | − | chr22 | 33215229 | 33215341 | + | chr8 | 139105402 | 139105659 | − |
| chr15 | 79201876 | 79202108 | − | chr22 | 35072284 | 35072492 | − | chr8 | 139302271 | 139302552 | − |
| chr15 | 79532676 | 79533136 | − | chr22 | 35228976 | 35229445 | − | chr8 | 139568805 | 139569137 | − |
| chr15 | 79607125 | 79607522 | + | chr22 | 36042353 | 36042638 | + | chr8 | 139583839 | 139584056 | + |
| chr15 | 79811871 | 79812105 | − | chr22 | 36042934 | 36043159 | + | chr8 | 139730132 | 139730228 | + |
| chr15 | 81081400 | 81081848 | − | chr22 | 36104273 | 36104742 | + | chr8 | 141040908 | 141041284 | + |
| chr15 | 81093918 | 81094391 | + | chr22 | 36422598 | 36422715 | + | chr8 | 141101294 | 141101515 | + |
| chr15 | 81161164 | 81161492 | − | chr22 | 36422785 | 36422906 | + | chr8 | 141101634 | 141101788 | + |
| chr15 | 81689354 | 81689612 | + | chr22 | 36743138 | 36743462 | + | chr8 | 141511511 | 141511672 | + |
| chr15 | 81690138 | 81690240 | + | chr22 | 36788024 | 36788431 | − | chr8 | 141520009 | 141520239 | − |
| chr15 | 81808529 | 81808828 | − | chr22 | 36981667 | 36981754 | + | chr8 | 141546752 | 141547016 | − |
| chr15 | 83479375 | 83479778 | − | chr22 | 36982088 | 36982223 | + | chr8 | 141557242 | 141557343 | − |
| chr15 | 83492473 | 83492955 | + | chr22 | 37097247 | 37097595 | − | chr8 | 141947157 | 141947593 | + |
| chr15 | 87216842 | 87217151 | + | chr22 | 37233613 | 37233913 | − | chr8 | 141991799 | 141991908 | + |
| chr15 | 87645693 | 87646143 | + | chr22 | 40979883 | 40980100 | − | chr8 | 141999208 | 141999630 | + |
| chr15 | 88202945 | 88203296 | + | chr22 | 40982847 | 40983074 | − | chr8 | 142147902 | 142148019 | − |
| chr15 | 88652723 | 88652995 | − | chr22 | 41032850 | 41032929 | − | chr8 | 143161840 | 143162179 | − |
| chr15 | 90224363 | 90224579 | − | chr22 | 41033228 | 41033300 | − | chr8_KI270817v1_alt | 157425 | 157764 | − |
| chr15 | 91148470 | 91148590 | − | chr22 | 41034343 | 41034658 | − | chr8_KI270821v1_alt | 852193 | 852308 | + |
| chr15 | 91149130 | 91149321 | − | chr22 | 44186660 | 44187083 | − | chr9 | 1238950 | 1239117 | + |
| chr15 | 92704328 | 92704633 | + | chr22 | 46012028 | 46012079 | + | chr9 | 1239599 | 1239661 | + |
| chr15 | 92705717 | 92705763 | + | chr22 | 47853328 | 47853507 | − | chr9 | 1439016 | 1439156 | − |
| chr15 | 93657875 | 93658201 | − | chr22 | 48347069 | 48347373 | + | chr9 | 1439452 | 1439813 | − |
| chr15 | 94540820 | 94540928 | − | chr22 | 48564008 | 48564079 | − | chr9 | 1601380 | 1601469 | − |
| chr15 | 94541170 | 94541269 | − | chr22 | 48564538 | 48564734 | − | chr9 | 2038334 | 2038794 | + |
| chr15 | 98122522 | 98122595 | − | chr22 | 49677315 | 49677680 | − | chr9 | 2217288 | 2217647 | − |
| chr15 | 98122974 | 98123161 | − | chr22 | 50593038 | 50593156 | − | chr9 | 2472891 | 2473344 | − |
| chr15 | 98131003 | 98131127 | + | chr22_KI270877v1_alt | 8811 | 9218 | − | chr9 | 2852708 | 2853160 | − |
| chr15 | 98131195 | 98131561 | + | chr2_KI270769v1_alt | 52751 | 52863 | + | chr9 | 3372035 | 3372487 | + |
| chr15 | 98144392 | 98144619 | − | chr2_KI270772v1_alt | 84758 | 85010 | − | chr9 | 4888581 | 4888696 | + |
| chr15 | 99032887 | 99033168 | − | chr2_KI270775v1_alt | 25428 | 25839 | + | chr9 | 4888709 | 4888746 | + |
| chr15 | 99051497 | 99051842 | + | chr2_KI270894v1_alt | 24614 | 24698 | − | chr9 | 5612383 | 5612776 | + |
| chr15 | 99074402 | 99074760 | − | chr2_KI270894v1_alt | 130383 | 130635 | + | chr9 | 6581012 | 6581182 | + |
| chr15 | 99849276 | 99849694 | + | chr3 | 679899 | 680030 | − | chr9 | 6581200 | 6581477 | + |
| chr15 | 100134123 | 100134364 | + | chr3 | 1036539 | 1037015 | − | chr9 | 7330663 | 7331138 | + |
| chr15 | 101950085 | 101950227 | + | chr3 | 1117693 | 1118054 | + | chr9 | 7403914 | 7404384 | + |
| chr15_GL383555v2_alt | 91722 | 91755 | − | chr3 | 1130243 | 1130729 | + | chr9 | 7442186 | 7442587 | + |
| chr15_GL383555v2_alt | 91828 | 91950 | − | chr3 | 1168228 | 1168498 | − | chr9 | 7443077 | 7443203 | + |
| chr15_GL383555v2_alt | 227336 | 227724 | − | chr3 | 1630244 | 1630563 | + | chr9 | 7504031 | 7504303 | − |
| chr15_KI270727v1_random | 282765 | 283197 | − | chr3 | 2420101 | 2420433 | + | chr9 | 11021452 | 11021743 | − |
| chr15_KI270852v1_alt | 313516 | 313948 | − | chr3 | 2420732 | 2420842 | + | chr9 | 11993578 | 11994007 | − |
| chr15_KI270905v1_alt | 4402197 | 4402498 | − | chr3 | 3656246 | 3656574 | + | chr9 | 12218253 | 12218431 | + |
| chr16 | 1062679 | 1062825 | − | chr3 | 3657512 | 3657624 | + | chr9 | 12396965 | 12397140 | + |
| chr16 | 5022687 | 5023045 | + | chr3 | 3741977 | 3742484 | − | chr9 | 12397208 | 12397333 | + |
| chr16 | 5153208 | 5153511 | − | chr3 | 3862536 | 3862779 | − | chr9 | 13463744 | 13463803 | + |
| chr16 | 5235574 | 5235819 | + | chr3 | 3863254 | 3863416 | − | chr9 | 13464105 | 13464332 | + |
| chr16 | 5364981 | 5365160 | − | chr3 | 3881219 | 3881520 | + | chr9 | 14481234 | 14481483 | + |
| chr16 | 5367311 | 5367390 | − | chr3 | 5349003 | 5349385 | + | chr9 | 16045154 | 16045331 | − |
| chr16 | 5376854 | 5377057 | + | chr3 | 5432001 | 5432431 | + | chr9 | 19701367 | 19701482 | + |
| chr16 | 5910714 | 5911102 | + | chr3 | 5687920 | 5688374 | + | chr9 | 19920404 | 19920716 | − |
| chr16 | 6010112 | 6010593 | + | chr3 | 5748092 | 5748351 | − | chr9 | 22268377 | 22268655 | + |
| chr16 | 6713911 | 6713966 | − | chr3 | 5756719 | 5757082 | − | chr9 | 23266301 | 23266757 | + |
| chr16 | 6714323 | 6714571 | − | chr3 | 7536226 | 7536442 | − | chr9 | 23621946 | 23622409 | − |
| chr16 | 6717873 | 6718070 | + | chr3 | 7536958 | 7537110 | − | chr9 | 24879126 | 24879470 | + |
| chr16 | 6731412 | 6731876 | + | chr3 | 7770109 | 7770509 | − | chr9 | 25035977 | 25036149 | − |
| chr16 | 6945935 | 6946150 | + | chr3 | 7964971 | 7965287 | + | chr9 | 25036536 | 25036577 | − |
| chr16 | 6946399 | 6946579 | + | chr3 | 8054943 | 8055379 | − | chr9 | 25261529 | 25262004 | + |
| chr16 | 7069540 | 7069929 | + | chr3 | 8115235 | 8115452 | − | chr9 | 25809195 | 25809443 | − |
| chr16 | 7995543 | 7996011 | − | chr3 | 8137143 | 8137609 | − | chr9 | 27307352 | 27307420 | + |
| chr16 | 8361912 | 8362138 | − | chr3 | 8279153 | 8279462 | − | chr9 | 28290324 | 28290444 | + |
| chr16 | 8597190 | 8597426 | + | chr3 | 8358001 | 8358464 | − | chr9 | 28290898 | 28291234 | + |
| chr16 | 8597728 | 8597784 | + | chr3 | 8702586 | 8702761 | − | chr9 | 28393702 | 28394118 | + |
| chr16 | 8666102 | 8666461 | + | chr3 | 8713921 | 8714370 | − | chr9 | 29007463 | 29007662 | + |
| chr16 | 9169091 | 9169181 | + | chr3 | 10548416 | 10548465 | + | chr9 | 29362822 | 29363271 | + |
| chr16 | 9221238 | 9221569 | + | chr3 | 10548492 | 10548771 | + | chr9 | 31697885 | 31698292 | − |
| chr16 | 9325615 | 9326105 | + | chr3 | 10713747 | 10714110 | + | chr9 | 32764885 | 32765330 | − |
| chr16 | 9361835 | 9361908 | + | chr3 | 10765784 | 10766234 | − | chr9 | 32801316 | 32801769 | − |
| chr16 | 9362312 | 9362433 | + | chr3 | 10774361 | 10774590 | − | chr9 | 33007792 | 33008037 | + |
| chr16 | 9362800 | 9362930 | + | chr3 | 10963495 | 10963599 | − | chr9 | 33231421 | 33231885 | + |
| chr16 | 9438790 | 9439105 | + | chr3 | 10964124 | 10964357 | − | chr9 | 34563176 | 34563624 | + |
| chr16 | 10047572 | 10048052 | + | chr3 | 10966914 | 10966977 | − | chr9 | 35628541 | 35628845 | + |
| chr16 | 10116556 | 10117036 | − | chr3 | 10981010 | 10981461 | + | chr9 | 37361453 | 37361654 | − |
| chr16 | 10136827 | 10137097 | − | chr3 | 10982700 | 10982764 | − | chr9 | 38027473 | 38027880 | − |
| chr16 | 10839268 | 10839340 | + | chr3 | 11116422 | 11116732 | + | chr9 | 38149533 | 38149754 | − |
| chr16 | 10930723 | 10930885 | + | chr3 | 11119380 | 11119794 | + | chr9 | 38150084 | 38150465 | + |
| chr16 | 11212197 | 11212461 | + | chr3 | 11975119 | 11975291 | − | chr9 | 40327130 | 40327436 | + |
| chr16 | 11620811 | 11620905 | + | chr3 | 11975554 | 11975701 | − | chr9 | 40327442 | 40327522 | + |
| chr16 | 11621505 | 11621874 | + | chr3 | 11991929 | 11992265 | + | chr9 | 40643982 | 40644234 | + |
| chr16 | 11625236 | 11625265 | + | chr3 | 11994705 | 11994759 | + | chr9 | 41015529 | 41015809 | − |
| chr16 | 11625562 | 11625630 | + | chr3 | 12445948 | 12446396 | + | chr9 | 42952239 | 42952559 | + |
| chr16 | 12944796 | 12945255 | − | chr3 | 12803374 | 12803419 | + | chr9 | 42952551 | 42952631 | + |
| chr16 | 13007934 | 13008411 | + | chr3 | 12803760 | 12804166 | − | chr9 | 61705142 | 61705279 | − |
| chr16 | 13030151 | 13030595 | + | chr3 | 13167211 | 13167592 | − | chr9 | 63443210 | 63443493 | + |
| chr16 | 13199859 | 13200260 | + | chr3 | 13471193 | 13471384 | + | chr9 | 64467169 | 64467475 | + |
| chr16 | 13240918 | 13241233 | − | chr3 | 13471402 | 13471569 | + | chr9 | 64467481 | 64467561 | + |
| chr16 | 13241528 | 13241629 | − | chr3 | 13533812 | 13533930 | − | chr9 | 64767212 | 64767464 | + |
| chr16 | 13400103 | 13400449 | + | chr3 | 13534087 | 13534240 | − | chr9 | 65252685 | 65252937 | + |
| chr16 | 13450922 | 13451364 | + | chr3 | 14224413 | 14224586 | + | chr9 | 65769024 | 65769276 | − |
| chr16 | 13540233 | 13540687 | + | chr3 | 14224645 | 14224809 | + | chr9 | 66048783 | 66048863 | − |
| chr16 | 13760121 | 13760310 | − | chr3 | 14628785 | 14629087 | − | chr9 | 66048869 | 66049175 | − |
| chr16 | 13835189 | 13835517 | + | chr3 | 14636472 | 14636896 | − | chr9 | 68416301 | 68416581 | + |
| chr16 | 14340941 | 14341020 | − | chr3 | 15165651 | 15166017 | + | chr9 | 68634248 | 68634706 | + |
| chr16 | 14341351 | 14341597 | − | chr3 | 15361894 | 15362250 | + | chr9 | 68667709 | 68668071 | + |
| chr16 | 14341967 | 14342072 | − | chr3 | 15513513 | 15513713 | + | chr9 | 69989973 | 69990398 | − |
| chr16 | 14806443 | 14806543 | + | chr3 | 15999039 | 15999430 | + | chr9 | 70460843 | 70461096 | − |
| chr16 | 14806855 | 14807032 | + | chr3 | 15999643 | 15999708 | + | chr9 | 70461865 | 70462102 | − |
| chr16 | 14807205 | 14807412 | + | chr3 | 16071389 | 16071434 | − | chr9 | 71142303 | 71142440 | − |
| chr16 | 15190153 | 15190551 | + | chr3 | 16072977 | 16073394 | − | chr9 | 71848909 | 71848952 | − |
| chr16 | 15236750 | 15236961 | + | chr3 | 16152746 | 16153155 | + | chr9 | 71854708 | 71855091 | − |
| chr16 | 15237178 | 15237374 | + | chr3 | 16167951 | 16168395 | − | chr9 | 72229393 | 72229739 | − |
| chr16 | 15248837 | 15248874 | − | chr3 | 16184639 | 16185075 | − | chr9 | 73128538 | 73128848 | + |
| chr16 | 15249225 | 15249622 | − | chr3 | 16234232 | 16234690 | + | chr9 | 73209126 | 73209394 | − |
| chr16 | 15306820 | 15307176 | + | chr3 | 16372089 | 16372385 | − | chr9 | 73211029 | 73211170 | − |
| chr16 | 15321384 | 15321599 | − | chr3 | 16786739 | 16787100 | + | chr9 | 73212825 | 73212997 | + |
| chr16 | 15321772 | 15321949 | − | chr3 | 16797503 | 16797903 | − | chr9 | 73213001 | 73213110 | + |
| chr16 | 15322419 | 15322518 | − | chr3 | 16802920 | 16803283 | − | chr9 | 73528099 | 73528411 | − |
| chr16 | 16433970 | 16434071 | + | chr3 | 16809025 | 16809274 | − | chr9 | 74317223 | 74317457 | + |
| chr16 | 16434383 | 16434560 | + | chr3 | 17134033 | 17134226 | − | chr9 | 75209731 | 75209852 | − |
| chr16 | 16434733 | 16434940 | + | chr3 | 17138859 | 17139109 | − | chr9 | 75209888 | 75209993 | − |
| chr16 | 16447786 | 16447889 | − | chr3 | 17155884 | 17155938 | − | chr9 | 75290514 | 75290981 | + |
| chr16 | 16448828 | 16449184 | − | chr3 | 17155933 | 17156195 | − | chr9 | 75672822 | 75672998 | − |
| chr16 | 16507098 | 16507495 | + | chr3 | 17811341 | 17811489 | − | chr9 | 75722661 | 75722879 | + |
| chr16 | 16507846 | 16507883 | + | chr3 | 18028455 | 18028900 | + | chr9 | 75730042 | 75730174 | − |
| chr16 | 16519347 | 16519543 | − | chr3 | 18064609 | 18064850 | − | chr9 | 76210070 | 76210428 | − |
| chr16 | 16519760 | 16519971 | − | chr3 | 18157817 | 18158310 | − | chr9 | 76578983 | 76579076 | + |
| chr16 | 16933737 | 16934177 | − | chr3 | 18164580 | 18165013 | + | chr9 | 76579088 | 76579211 | + |
| chr16 | 17000208 | 17000446 | + | chr3 | 18529396 | 18529867 | − | chr9 | 76824996 | 76825129 | + |
| chr16 | 17495944 | 17496344 | − | chr3 | 18577505 | 18577961 | + | chr9 | 76825351 | 76825496 | + |
| chr16 | 17950616 | 17950775 | + | chr3 | 18685068 | 18685291 | + | chr9 | 77091662 | 77091930 | − |
| chr16 | 17951338 | 17951369 | + | chr3 | 18785294 | 18785447 | − | chr9 | 78004077 | 78004213 | + |
| chr16 | 17951394 | 17951593 | + | chr3 | 18785821 | 18785991 | − | chr9 | 78004521 | 78004674 | + |
| chr16 | 17983968 | 17984395 | + | chr3 | 18823526 | 18823679 | + | chr9 | 78075321 | 78075406 | − |
| chr16 | 18009083 | 18009233 | − | chr3 | 18837167 | 18837564 | − | chr9 | 78081732 | 78081992 | − |
| chr16 | 18009281 | 18009616 | − | chr3 | 19005653 | 19006125 | + | chr9 | 78119099 | 78119205 | − |
| chr16 | 18059544 | 18059981 | + | chr3 | 19022994 | 19023326 | − | chr9 | 78191813 | 78191919 | − |
| chr16 | 18101754 | 18102157 | + | chr3 | 19126366 | 19126617 | − | chr9 | 78991491 | 78991901 | − |
| chr16 | 18102675 | 18102692 | + | chr3 | 19126973 | 19127101 | − | chr9 | 79049505 | 79049862 | + |
| chr16 | 18192214 | 18192425 | + | chr3 | 19252817 | 19253258 | + | chr9 | 79275831 | 79275978 | − |
| chr16 | 18192642 | 18192838 | + | chr3 | 19518856 | 19519113 | − | chr9 | 79276310 | 79276536 | − |
| chr16 | 18204308 | 18204345 | − | chr3 | 19542534 | 19542851 | − | chr9 | 79282287 | 79282426 | − |
| chr16 | 18204700 | 18205092 | − | chr3 | 20093121 | 20093539 | − | chr9 | 79915599 | 79915773 | + |
| chr16 | 18262688 | 18263044 | + | chr3 | 21085873 | 21086199 | + | chr9 | 80430607 | 80430744 | − |
| chr16 | 18263997 | 18264100 | + | chr3 | 21086565 | 21086676 | + | chr9 | 80709993 | 80710446 | + |
| chr16 | 18276909 | 18277116 | − | chr3 | 21434186 | 21434356 | + | chr9 | 82120052 | 82120126 | − |
| chr16 | 18277289 | 18277466 | − | chr3 | 22114432 | 22114728 | − | chr9 | 82120190 | 82120307 | − |
| chr16 | 18277777 | 18277879 | − | chr3 | 22115080 | 22115173 | − | chr9 | 82202384 | 82202581 | − |
| chr16 | 19136015 | 19136425 | + | chr3 | 22225242 | 22225769 | + | chr9 | 82202889 | 82202963 | − |
| chr16 | 19193602 | 19194007 | − | chr3 | 22735558 | 22735690 | + | chr9 | 82203021 | 82203128 | − |
| chr16 | 19261894 | 19262027 | − | chr3 | 22861948 | 22862350 | − | chr9 | 82266145 | 82266521 | + |
| chr16 | 19346359 | 19346602 | − | chr3 | 22874961 | 22875152 | − | chr9 | 82351620 | 82351987 | + |
| chr16 | 19652885 | 19653223 | − | chr3 | 23081419 | 23081777 | + | chr9 | 82552143 | 82552496 | + |
| chr16 | 19984903 | 19984961 | + | chr3 | 23083961 | 23084388 | − | chr9 | 82552697 | 82552777 | + |
| chr16 | 22887943 | 22888215 | + | chr3 | 23775239 | 23775651 | + | chr9 | 82777581 | 82777849 | − |
| chr16 | 23759831 | 23760040 | + | chr3 | 23869515 | 23869608 | − | chr9 | 82777886 | 82777994 | − |
| chr16 | 23760327 | 23760337 | + | chr3 | 23869742 | 23869845 | − | chr9 | 83308748 | 83309194 | + |
| chr16 | 23760371 | 23760503 | + | chr3 | 23870432 | 23870578 | − | chr9 | 84061430 | 84061818 | − |
| chr16 | 23959915 | 23960154 | + | chr3 | 24479766 | 24480213 | + | chr9 | 84173054 | 84173170 | + |
| chr16 | 23960730 | 23960868 | + | chr3 | 24587936 | 24588346 | + | chr9 | 84181475 | 84181636 | − |
| chr16 | 24117627 | 24118034 | − | chr3 | 24792320 | 24792667 | + | chr9 | 84181648 | 84181857 | − |
| chr16 | 24506922 | 24506997 | − | chr3 | 25055644 | 25055813 | − | chr9 | 84220340 | 84220382 | + |
| chr16 | 24507403 | 24507517 | − | chr3 | 25055826 | 25055951 | − | chr9 | 84220694 | 84220845 | + |
| chr16 | 24507802 | 24507815 | − | chr3 | 25407661 | 25408126 | + | chr9 | 84221227 | 84221269 | + |
| chr16 | 24508124 | 24508352 | − | chr3 | 25530949 | 25531348 | − | chr9 | 84853682 | 84853801 | + |
| chr16 | 25444516 | 25444881 | + | chr3 | 25573010 | 25573277 | + | chr9 | 84854102 | 84854429 | + |
| chr16 | 25887222 | 25887374 | − | chr3 | 25603631 | 25603902 | − | chr9 | 84855687 | 84855764 | + |
| chr16 | 25887547 | 25887686 | − | chr3 | 25957137 | 25957572 | + | chr9 | 85226363 | 85226480 | + |
| chr16 | 25953640 | 25954095 | − | chr3 | 25971965 | 25972335 | + | chr9 | 85226780 | 85227126 | + |
| chr16 | 26080420 | 26080902 | − | chr3 | 25973722 | 25974016 | − | chr9 | 85272991 | 85273471 | − |
| chr16 | 26643594 | 26643912 | − | chr3 | 25974442 | 25974474 | − | chr9 | 85292174 | 85292606 | + |
| chr16 | 26649232 | 26649475 | + | chr3 | 26098096 | 26098304 | + | chr9 | 85298863 | 85299080 | − |
| chr16 | 26673819 | 26673913 | + | chr3 | 26098751 | 26098968 | + | chr9 | 85394487 | 85394925 | + |
| chr16 | 26673947 | 26674047 | + | chr3 | 26576987 | 26577398 | + | chr9 | 85505666 | 85506032 | − |
| chr16 | 26674879 | 26674935 | + | chr3 | 26595632 | 26595972 | − | chr9 | 85508682 | 85509178 | − |
| chr16 | 26774842 | 26775148 | + | chr3 | 26596206 | 26596515 | − | chr9 | 85525119 | 85525430 | − |
| chr16 | 27027870 | 27028353 | + | chr3 | 26616359 | 26616633 | − | chr9 | 86175165 | 86175292 | − |
| chr16 | 27047625 | 27048098 | + | chr3 | 26664535 | 26664849 | − | chr9 | 86176028 | 86176127 | − |
| chr16 | 27302842 | 27303020 | − | chr3 | 26716556 | 26717011 | + | chr9 | 86771362 | 86771568 | − |
| chr16 | 27364897 | 27365010 | − | chr3 | 26819608 | 26819701 | + | chr9 | 86905851 | 86906108 | − |
| chr16 | 27365554 | 27365748 | − | chr3 | 26982557 | 26982997 | − | chr9 | 87039910 | 87040187 | − |
| chr16 | 27388735 | 27389131 | + | chr3 | 27027423 | 27027866 | − | chr9 | 87040601 | 87040628 | − |
| chr16 | 27717679 | 27718100 | + | chr3 | 27086881 | 27087299 | − | chr9 | 87040621 | 87040722 | − |
| chr16 | 27806404 | 27806742 | − | chr3 | 27114906 | 27115287 | + | chr9 | 87075577 | 87075913 | − |
| chr16 | 27826468 | 27826719 | − | chr3 | 27241300 | 27241750 | + | chr9 | 87107955 | 87108228 | − |
| chr16 | 27875204 | 27875496 | + | chr3 | 27253931 | 27254099 | + | chr9 | 87141583 | 87141960 | − |
| chr16 | 28036414 | 28036677 | + | chr3 | 27254306 | 27254490 | + | chr9 | 87197298 | 87197741 | − |
| chr16 | 28283832 | 28284237 | + | chr3 | 27508002 | 27508417 | + | chr9 | 87469584 | 87469893 | − |
| chr16 | 28999549 | 28999937 | − | chr3 | 27570502 | 27570995 | − | chr9 | 87469905 | 87470102 | − |
| chr16 | 29257563 | 29257702 | − | chr3 | 27646364 | 27646703 | + | chr9 | 87638913 | 87639207 | + |
| chr16 | 31255650 | 31255752 | + | chr3 | 27668295 | 27668381 | − | chr9 | 88665621 | 88665676 | − |
| chr16 | 31332914 | 31332978 | − | chr3 | 27747945 | 27748345 | − | chr9 | 88666080 | 88666370 | − |
| chr16 | 31333611 | 31333903 | − | chr3 | 27773100 | 27773551 | + | chr9 | 88680638 | 88680744 | + |
| chr16 | 31481204 | 31481554 | + | chr3 | 27777434 | 27777762 | + | chr9 | 89008685 | 89008957 | + |
| chr16 | 31481837 | 31481976 | + | chr3 | 27778305 | 27778409 | + | chr9 | 89106655 | 89106763 | + |
| chr16 | 46711680 | 46711901 | + | chr3 | 27829153 | 27829662 | + | chr9 | 89633245 | 89633509 | + |
| chr16 | 46712454 | 46712602 | + | chr3 | 27841720 | 27842177 | + | chr9 | 89793415 | 89793459 | + |
| chr16 | 47031755 | 47031942 | − | chr3 | 27936150 | 27936253 | + | chr9 | 89992394 | 89992529 | + |
| chr16 | 47878539 | 47878966 | + | chr3 | 27936638 | 27936732 | + | chr9 | 90015802 | 90016143 | − |
| chr16 | 48066910 | 48067101 | + | chr3 | 28080059 | 28080299 | − | chr9 | 90066703 | 90066774 | − |
| chr16 | 48067470 | 48067705 | + | chr3 | 28186212 | 28186619 | + | chr9 | 90109675 | 90110100 | + |
| chr16 | 48092237 | 48092513 | − | chr3 | 28198370 | 28198640 | + | chr9 | 90134361 | 90134478 | − |
| chr16 | 48108881 | 48109175 | − | chr3 | 28537464 | 28537816 | − | chr9 | 90340338 | 90340426 | − |
| chr16 | 48125060 | 48125372 | − | chr3 | 28558471 | 28558778 | − | chr9 | 90340501 | 90340681 | − |
| chr16 | 48697975 | 48698266 | + | chr3 | 28747694 | 28748119 | − | chr9 | 90772221 | 90772556 | − |
| chr16 | 48743343 | 48743528 | − | chr3 | 28760405 | 28760773 | + | chr9 | 90935701 | 90935759 | − |
| chr16 | 49061634 | 49061726 | + | chr3 | 28802127 | 28802571 | + | chr9 | 90936058 | 90936390 | − |
| chr16 | 49295956 | 49296387 | − | chr3 | 28985423 | 28985864 | − | chr9 | 91102186 | 91102380 | − |
| chr16 | 49327118 | 49327436 | + | chr3 | 29178865 | 29178929 | + | chr9 | 91488036 | 91488245 | − |
| chr16 | 49893607 | 49893731 | − | chr3 | 29178931 | 29179297 | + | chr9 | 91556181 | 91556549 | + |
| chr16 | 49897020 | 49897132 | + | chr3 | 29634487 | 29634760 | + | chr9 | 91853944 | 91854289 | − |
| chr16 | 49897439 | 49897576 | + | chr3 | 29696759 | 29696904 | − | chr9 | 92004048 | 92004233 | + |
| chr16 | 50114619 | 50115056 | + | chr3 | 29773643 | 29774047 | + | chr9 | 92117861 | 92117892 | + |
| chr16 | 50384991 | 50385180 | + | chr3 | 30077318 | 30077474 | + | chr9 | 92121600 | 92121716 | + |
| chr16 | 50385251 | 50385531 | + | chr3 | 30077515 | 30077756 | + | chr9 | 93042650 | 93042795 | − |
| chr16 | 50400158 | 50400267 | − | chr3 | 30078225 | 30078298 | + | chr9 | 93048150 | 93048234 | − |
| chr16 | 50400468 | 50400783 | − | chr3 | 30115020 | 30115493 | − | chr9 | 93699655 | 93699757 | − |
| chr16 | 50401748 | 50401883 | + | chr3 | 30152352 | 30152597 | − | chr9 | 93699778 | 93699965 | − |
| chr16 | 50455301 | 50455409 | + | chr3 | 30269977 | 30270374 | − | chr9 | 93731572 | 93731836 | + |
| chr16 | 50455436 | 50455631 | + | chr3 | 30400475 | 30400859 | + | chr9 | 93943235 | 93943514 | − |
| chr16 | 50456254 | 50456567 | − | chr3 | 30681797 | 30682111 | − | chr9 | 94689311 | 94689743 | + |
| chr16 | 50530254 | 50530450 | − | chr3 | 30723907 | 30724295 | + | chr9 | 94690936 | 94690977 | + |
| chr16 | 50602073 | 50602141 | − | chr3 | 30876609 | 30876928 | + | chr9 | 94712026 | 94712470 | − |
| chr16 | 50647445 | 50647679 | − | chr3 | 31009202 | 31009665 | − | chr9 | 95290442 | 95290646 | − |
| chr16 | 50647701 | 50647792 | − | chr3 | 31051131 | 31051465 | − | chr9 | 95534441 | 95534552 | + |
| chr16 | 50677000 | 50677290 | + | chr3 | 31077767 | 31078233 | − | chr9 | 95539758 | 95540209 | + |
| chr16 | 50725712 | 50726065 | − | chr3 | 31103488 | 31103762 | − | chr9 | 95575778 | 95576120 | − |
| chr16 | 50841931 | 50842237 | − | chr3 | 31170937 | 31171388 | + | chr9 | 95578228 | 95578702 | + |
| chr16 | 50879056 | 50879140 | + | chr3 | 31424149 | 31424245 | − | chr9 | 95594929 | 95595348 | − |
| chr16 | 50894019 | 50894274 | + | chr3 | 31505445 | 31505813 | − | chr9 | 95603745 | 95603850 | + |
| chr16 | 50900695 | 50901107 | + | chr3 | 32223269 | 32223572 | − | chr9 | 95604713 | 95604957 | + |
| chr16 | 50982272 | 50982703 | − | chr3 | 32594555 | 32594709 | − | chr9 | 95745848 | 95746282 | + |
| chr16 | 51058348 | 51058793 | − | chr3 | 32595016 | 32595295 | − | chr9 | 95839007 | 95839395 | + |
| chr16 | 51091573 | 51091636 | − | chr3 | 33207272 | 33207743 | + | chr9 | 96215892 | 96216120 | + |
| chr16 | 51198775 | 51198837 | − | chr3 | 33730844 | 33731140 | + | chr9 | 96605210 | 96605663 | + |
| chr16 | 51198877 | 51199027 | − | chr3 | 34104706 | 34105164 | − | chr9 | 97739321 | 97739647 | + |
| chr16 | 51285579 | 51285906 | − | chr3 | 34222402 | 34222849 | − | chr9 | 97823072 | 97823480 | − |
| chr16 | 51306875 | 51306933 | − | chr3 | 34357946 | 34358293 | − | chr9 | 98402500 | 98402808 | − |
| chr16 | 51307289 | 51307595 | − | chr3 | 34408510 | 34408827 | − | chr9 | 98642991 | 98643225 | − |
| chr16 | 51452620 | 51453054 | + | chr3 | 34493841 | 34494280 | − | chr9 | 98939197 | 98939651 | − |
| chr16 | 51515735 | 51516017 | + | chr3 | 34640078 | 34640453 | + | chr9 | 99076367 | 99076756 | − |
| chr16 | 51697544 | 51697736 | + | chr3 | 34642483 | 34642558 | + | chr9 | 99353805 | 99354017 | + |
| chr16 | 52567035 | 52567344 | − | chr3 | 34810810 | 34811041 | + | chr9 | 99397438 | 99397599 | + |
| chr16 | 52567947 | 52568089 | − | chr3 | 34811067 | 34811140 | + | chr9 | 99398082 | 99398415 | + |
| chr16 | 52674475 | 52674880 | + | chr3 | 34811545 | 34811624 | + | chr9 | 100070835 | 100070946 | − |
| chr16 | 52766743 | 52767088 | − | chr3 | 36756041 | 36756486 | − | chr9 | 100921351 | 100921645 | + |
| chr16 | 52823419 | 52823561 | + | chr3 | 36762319 | 36762725 | − | chr9 | 101158420 | 101158875 | − |
| chr16 | 52824354 | 52824516 | + | chr3 | 37223350 | 37223766 | + | chr9 | 101200063 | 101200454 | + |
| chr16 | 52824524 | 52824646 | + | chr3 | 37437106 | 37437558 | − | chr9 | 101202530 | 101202968 | − |
| chr16 | 52897826 | 52898274 | + | chr3 | 37483655 | 37483795 | − | chr9 | 102019221 | 102019686 | + |
| chr16 | 53061426 | 53061710 | − | chr3 | 37539230 | 37539353 | − | chr9 | 102943356 | 102943653 | + |
| chr16 | 53552077 | 53552496 | + | chr3 | 37539766 | 37540096 | − | chr9 | 103277174 | 103277565 | + |
| chr16 | 54313683 | 54313908 | + | chr3 | 37666206 | 37666312 | − | chr9 | 104038540 | 104038885 | − |
| chr16 | 54357445 | 54357744 | + | chr3 | 37666861 | 37667096 | − | chr9 | 105879007 | 105879464 | + |
| chr16 | 54433204 | 54433647 | + | chr3 | 37675047 | 37675495 | + | chr9 | 106050757 | 106051008 | + |
| chr16 | 54433941 | 54433989 | + | chr3 | 37676369 | 37676566 | − | chr9 | 106137979 | 106138035 | − |
| chr16 | 54715033 | 54715462 | − | chr3 | 37687789 | 37688167 | − | chr9 | 106138511 | 106138855 | − |
| chr16 | 54748966 | 54749326 | − | chr3 | 37690752 | 37691212 | − | chr9 | 107765703 | 107765984 | − |
| chr16 | 54763003 | 54763197 | + | chr3 | 37701284 | 37701688 | − | chr9 | 108091428 | 108091765 | + |
| chr16 | 54763167 | 54763308 | + | chr3 | 37719527 | 37719750 | + | chr9 | 110567824 | 110568211 | + |
| chr16 | 54844034 | 54844094 | − | chr3 | 37741107 | 37741540 | + | chr9 | 112762677 | 112763157 | − |
| chr16 | 54844392 | 54844611 | − | chr3 | 37817364 | 37817621 | − | chr9 | 113697777 | 113697986 | + |
| chr16 | 54845140 | 54845264 | − | chr3 | 38158523 | 38158986 | + | chr9 | 113698369 | 113698524 | + |
| chr16 | 55051605 | 55052049 | + | chr3 | 38798081 | 38798355 | − | chr9 | 114561231 | 114561329 | − |
| chr16 | 55147891 | 55148152 | + | chr3 | 38907153 | 38907302 | + | chr9 | 114561628 | 114561989 | − |
| chr16 | 55210296 | 55210562 | − | chr3 | 38907411 | 38907595 | + | chr9 | 116113670 | 116113895 | + |
| chr16 | 55351595 | 55351919 | − | chr3 | 39020466 | 39020747 | + | chr9 | 116114637 | 116114878 | + |
| chr16 | 55423374 | 55423757 | − | chr3 | 39167986 | 39168151 | + | chr9 | 116349896 | 116350176 | − |
| chr16 | 55652614 | 55653065 | + | chr3 | 39168179 | 39168390 | + | chr9 | 116407722 | 116408161 | − |
| chr16 | 55671090 | 55671422 | − | chr3 | 39197184 | 39197308 | − | chr9 | 117265099 | 117265578 | − |
| chr16 | 55743073 | 55743538 | − | chr3 | 39198427 | 39198502 | − | chr9 | 117310463 | 117310617 | + |
| chr16 | 55786250 | 55786716 | − | chr3 | 39198926 | 39199033 | − | chr9 | 117310919 | 117311135 | + |
| chr16 | 55809907 | 55810373 | + | chr3 | 39391107 | 39391387 | + | chr9 | 117561637 | 117562016 | − |
| chr16 | 55910743 | 55911019 | + | chr3 | 39446061 | 39446175 | − | chr9 | 117620383 | 117620842 | + |
| chr16 | 56061292 | 56061726 | + | chr3 | 39506367 | 39506823 | − | chr9 | 118088132 | 118088542 | − |
| chr16 | 56089803 | 56090007 | + | chr3 | 39509777 | 39509863 | + | chr9 | 118580917 | 118581171 | − |
| chr16 | 56326433 | 56326697 | − | chr3 | 39753196 | 39753618 | − | chr9 | 118789243 | 118789552 | − |
| chr16 | 56368001 | 56368344 | + | chr3 | 39768072 | 39768547 | + | chr9 | 119611532 | 119611935 | − |
| chr16 | 56569217 | 56569606 | + | chr3 | 40186298 | 40186770 | − | chr9 | 119731067 | 119731546 | + |
| chr16 | 56831412 | 56831519 | − | chr3 | 40213699 | 40214141 | + | chr9 | 120091733 | 120092160 | − |
| chr16 | 56831531 | 56831725 | − | chr3 | 40250626 | 40251065 | − | chr9 | 120183186 | 120183477 | − |
| chr16 | 56881558 | 56881719 | − | chr3 | 40266148 | 40266328 | + | chr9 | 120183510 | 120183558 | − |
| chr16 | 58219248 | 58219493 | − | chr3 | 40319625 | 40320144 | + | chr9 | 122501756 | 122502208 | − |
| chr16 | 59027770 | 59028076 | − | chr3 | 40333981 | 40334440 | + | chr9 | 123304069 | 123304305 | − |
| chr16 | 59192664 | 59192955 | − | chr3 | 40395404 | 40395866 | − | chr9 | 124073438 | 124073568 | + |
| chr16 | 59385009 | 59385416 | − | chr3 | 40590264 | 40590514 | + | chr9 | 124073877 | 124074135 | + |
| chr16 | 59385463 | 59385488 | − | chr3 | 40590826 | 40590977 | + | chr9 | 124206218 | 124206683 | − |
| chr16 | 59385856 | 59385984 | + | chr3 | 40618064 | 40618496 | − | chr9 | 125394412 | 125394651 | + |
| chr16 | 59459521 | 59459915 | − | chr3 | 40640359 | 40640505 | − | chr9 | 125395969 | 125396094 | + |
| chr16 | 59539451 | 59539589 | − | chr3 | 40641624 | 40641887 | − | chr9 | 126148296 | 126148638 | − |
| chr16 | 59540492 | 59540757 | − | chr3 | 40651811 | 40651904 | − | chr9 | 130554795 | 130554903 | + |
| chr16 | 59751902 | 59752135 | − | chr3 | 40651890 | 40652015 | − | chr9 | 130950895 | 130951158 | + |
| chr16 | 59803445 | 59803829 | − | chr3 | 40663466 | 40663737 | + | chr9 | 133202263 | 133202630 | + |
| chr16 | 59847005 | 59847125 | − | chr3 | 40738801 | 40739072 | − | chr9 | 133630186 | 133630476 | + |
| chr16 | 59847529 | 59847702 | − | chr3 | 40746159 | 40746543 | + | chr9 | 134269202 | 134269316 | − |
| chr16 | 60812392 | 60812768 | − | chr3 | 40769877 | 40770055 | + | chr9 | 134485668 | 134485905 | − |
| chr16 | 60835361 | 60835808 | − | chr3 | 40770259 | 40770419 | + | chr9 | 137592250 | 137592308 | − |
| chr16 | 60916290 | 60916541 | − | chr3 | 40786710 | 40786836 | + | chr9 | 137592600 | 137592672 | − |
| chr16 | 61000778 | 61000990 | + | chr3 | 40790007 | 40790410 | − | chr9_GL383539v1_alt | 7106 | 7491 | + |
| chr16 | 61053880 | 61054210 | − | chr3 | 40864074 | 40864466 | + | chr9_GL383539v1_alt | 7981 | 8107 | + |
| chr16 | 61054520 | 61054618 | − | chr3 | 40893229 | 40893658 | − | chr9_GL383539v1_alt | 28037 | 28422 | + |
| chr16 | 61083311 | 61083657 | − | chr3 | 40991750 | 40992046 | − | chr9_GL383539v1_alt | 28912 | 29038 | + |
| chr16 | 61230055 | 61230477 | + | chr3 | 40999246 | 40999638 | + | chr9_GL383539v1_alt | 89850 | 90122 | − |
| chr16 | 61306426 | 61306532 | − | chr3 | 41013420 | 41013864 | − | chr9_KI270717v1_random | 17642 | 17894 | + |
| chr16 | 61306847 | 61307078 | − | chr3 | 41058590 | 41058820 | + | chr9_KI270823v1_alt | 209623 | 210078 | − |
| chr16 | 61821849 | 61822205 | + | chr3 | 41074346 | 41074737 | + | chr9_KI270823v1_alt | 251266 | 251657 | + |
| chr16 | 61822246 | 61822364 | + | chr3 | 41118961 | 41119221 | − | chr9_KI270823v1_alt | 253733 | 254171 | − |
| chr16 | 62696582 | 62696995 | − | chr3 | 41121556 | 41121980 | − | chrUn_GL000224v1 | 73533 | 73848 | + |
| chr16 | 62918665 | 62919126 | + | chr3 | 41127905 | 41128342 | − | chrUn_KI270741v1 | 15428 | 15743 | + |
| chr16 | 63499883 | 63500306 | + | chr3 | 41147859 | 41147964 | + | chrX | 424415 | 424545 | + |
| chr16 | 63673646 | 63673947 | − | chr3 | 41148291 | 41148409 | + | chrX | 1905974 | 1906275 | − |
| chr16 | 63916933 | 63917205 | + | chr3 | 41163179 | 41163628 | − | chrX | 2597278 | 2597414 | − |
| chr16 | 64370740 | 64371020 | − | chr3 | 41188030 | 41188309 | − | chrX | 2597826 | 2597915 | − |
| chr16 | 65258271 | 65258324 | − | chr3 | 41998050 | 41998439 | − | chrX | 3055689 | 3055903 | + |
| chr16 | 65258740 | 65259145 | − | chr3 | 42272649 | 42273016 | − | chrX | 3365496 | 3365606 | − |
| chr16 | 65509247 | 65509622 | + | chr3 | 42278103 | 42278540 | + | chrX | 5071702 | 5072071 | + |
| chr16 | 65871778 | 65872164 | + | chr3 | 42352885 | 42353169 | − | chrX | 6861630 | 6861699 | − |
| chr16 | 66005446 | 66005835 | + | chr3 | 42353182 | 42353264 | − | chrX | 7676590 | 7676883 | − |
| chr16 | 66092079 | 66092559 | + | chr3 | 42372412 | 42372852 | − | chrX | 7756863 | 7757114 | + |
| chr16 | 66118209 | 66118472 | − | chr3 | 42477602 | 42477996 | + | chrX | 8033081 | 8033373 | − |
| chr16 | 66150413 | 66150702 | + | chr3 | 42844092 | 42844131 | − | chrX | 8294847 | 8295073 | + |
| chr16 | 66163054 | 66163411 | − | chr3 | 42844207 | 42844642 | − | chrX | 8296972 | 8297078 | + |
| chr16 | 66187156 | 66187567 | − | chr3 | 42997743 | 42998067 | − | chrX | 8500718 | 8501196 | − |
| chr16 | 66220610 | 66221059 | − | chr3 | 43023825 | 43023911 | − | chrX | 8579590 | 8579728 | + |
| chr16 | 66262177 | 66262380 | − | chr3 | 43035196 | 43035319 | + | chrX | 8579784 | 8580000 | + |
| chr16 | 66302686 | 66302841 | − | chr3 | 43035428 | 43035646 | + | chrX | 8879919 | 8880163 | − |
| chr16 | 66303605 | 66303718 | − | chr3 | 43048393 | 43048745 | − | chrX | 8880215 | 8880268 | − |
| chr16 | 66390741 | 66390833 | − | chr3 | 43082793 | 43082951 | − | chrX | 9180762 | 9181093 | + |
| chr16 | 66390854 | 66390999 | − | chr3 | 43093658 | 43093919 | + | chrX | 9300798 | 9300954 | + |
| chr16 | 66401545 | 66401781 | − | chr3 | 43120086 | 43120271 | − | chrX | 9386754 | 9387092 | + |
| chr16 | 66484601 | 66484893 | − | chr3 | 43120308 | 43120465 | − | chrX | 10171404 | 10171591 | − |
| chr16 | 66490517 | 66490684 | − | chr3 | 43148505 | 43148777 | + | chrX | 10172151 | 10172208 | − |
| chr16 | 66795274 | 66795549 | + | chr3 | 43149142 | 43149213 | + | chrX | 10172274 | 10172455 | − |
| chr16 | 67494892 | 67494968 | − | chr3 | 43157954 | 43158315 | + | chrX | 10248703 | 10248973 | − |
| chr16 | 67495874 | 67496180 | − | chr3 | 43161805 | 43162155 | + | chrX | 10349439 | 10349926 | − |
| chr16 | 67909859 | 67910354 | − | chr3 | 43636056 | 43636464 | − | chrX | 10506347 | 10506573 | − |
| chr16 | 70971077 | 70971303 | + | chr3 | 43761815 | 43762252 | − | chrX | 10527594 | 10527955 | + |
| chr16 | 72151754 | 72152185 | + | chr3 | 43792999 | 43793384 | + | chrX | 10656907 | 10657355 | + |
| chr16 | 72163512 | 72163660 | − | chr3 | 43840765 | 43841170 | + | chrX | 11186864 | 11187337 | − |
| chr16 | 72164073 | 72164330 | − | chr3 | 43898288 | 43898394 | − | chrX | 11457648 | 11457999 | + |
| chr16 | 73356808 | 73357186 | + | chr3 | 43898830 | 43898928 | − | chrX | 11526045 | 11526288 | − |
| chr16 | 73445641 | 73446067 | + | chr3 | 43948571 | 43948731 | − | chrX | 11536133 | 11536572 | + |
| chr16 | 73517651 | 73517750 | − | chr3 | 43962761 | 43963102 | + | chrX | 11567187 | 11567371 | − |
| chr16 | 73696026 | 73696331 | − | chr3 | 43963362 | 43963516 | + | chrX | 11576633 | 11576705 | − |
| chr16 | 73704621 | 73704895 | − | chr3 | 43965554 | 43965664 | + | chrX | 11577232 | 11577563 | − |
| chr16 | 73705295 | 73705503 | − | chr3 | 43967431 | 43967778 | + | chrX | 11600442 | 11600738 | + |
| chr16 | 73729031 | 73729249 | + | chr3 | 44073186 | 44073461 | − | chrX | 11600740 | 11601079 | − |
| chr16 | 73729561 | 73729651 | + | chr3 | 44110864 | 44111091 | − | chrX | 11821211 | 11821412 | + |
| chr16 | 74015641 | 74015697 | − | chr3 | 44123532 | 44123966 | + | chrX | 11871204 | 11871341 | + |
| chr16 | 74094654 | 74094740 | + | chr3 | 44127527 | 44127935 | + | chrX | 12278710 | 12278916 | + |
| chr16 | 74117676 | 74118034 | − | chr3 | 44127936 | 44127976 | − | chrX | 12278980 | 12279136 | + |
| chr16 | 74139046 | 74139351 | + | chr3 | 44130618 | 44130756 | + | chrX | 12413162 | 12413429 | + |
| chr16 | 74140159 | 74140170 | + | chr3 | 44134389 | 44134597 | − | chrX | 12463711 | 12463918 | − |
| chr16 | 74243236 | 74243506 | − | chr3 | 44134609 | 44134730 | − | chrX | 12647175 | 12647554 | − |
| chr16 | 74949528 | 74949649 | + | chr3 | 44146072 | 44146545 | − | chrX | 12679063 | 12679226 | + |
| chr16 | 75393180 | 75393382 | + | chr3 | 44166309 | 44166499 | + | chrX | 12679677 | 12679971 | + |
| chr16 | 75657896 | 75658053 | + | chr3 | 44166723 | 44166940 | + | chrX | 12691605 | 12691904 | − |
| chr16 | 75806314 | 75806711 | − | chr3 | 45211073 | 45211510 | − | chrX | 13299189 | 13299368 | − |
| chr16 | 76061155 | 76061625 | + | chr3 | 45818665 | 45818847 | + | chrX | 13428591 | 13429025 | + |
| chr16 | 76389862 | 76390143 | − | chr3 | 45822229 | 45822568 | + | chrX | 13484079 | 13484475 | + |
| chr16 | 76854393 | 76854784 | + | chr3 | 46270456 | 46270868 | − | chrX | 13552839 | 13553301 | + |
| chr16 | 77049323 | 77049557 | + | chr3 | 46854840 | 46855131 | + | chrX | 13567492 | 13567933 | + |
| chr16 | 77222971 | 77223202 | + | chr3 | 47223958 | 47224239 | − | chrX | 13573966 | 13574295 | − |
| chr16 | 77223872 | 77223927 | + | chr3 | 48226995 | 48227306 | + | chrX | 13580937 | 13581092 | + |
| chr16 | 77224253 | 77224294 | + | chr3 | 49170199 | 49170644 | + | chrX | 13604091 | 13604169 | − |
| chr16 | 77273919 | 77274142 | − | chr3 | 50594632 | 50594866 | + | chrX | 13604290 | 13604499 | − |
| chr16 | 77521818 | 77522207 | + | chr3 | 50595692 | 50595772 | + | chrX | 13610937 | 13611203 | + |
| chr16 | 77641864 | 77642135 | + | chr3 | 51836439 | 51836918 | − | chrX | 13621100 | 13621443 | − |
| chr16 | 77750072 | 77750510 | + | chr3 | 52176864 | 52177224 | − | chrX | 13621573 | 13621806 | − |
| chr16 | 77774411 | 77774806 | + | chr3 | 54156127 | 54156438 | − | chrX | 13959689 | 13959948 | − |
| chr16 | 77782246 | 77782717 | + | chr3 | 54156451 | 54156489 | − | chrX | 13984644 | 13984935 | − |
| chr16 | 77786043 | 77786385 | + | chr3 | 54857299 | 54857709 | + | chrX | 13990204 | 13990491 | − |
| chr16 | 77861677 | 77862043 | + | chr3 | 55116367 | 55116502 | + | chrX | 14126527 | 14126956 | + |
| chr16 | 77915777 | 77916136 | − | chr3 | 55230790 | 55231017 | − | chrX | 14149932 | 14150389 | + |
| chr16 | 77976996 | 77977453 | + | chr3 | 55234177 | 55234387 | + | chrX | 14553580 | 14553998 | + |
| chr16 | 77983244 | 77983405 | − | chr3 | 55234399 | 55234589 | + | chrX | 14671335 | 14671683 | − |
| chr16 | 77983426 | 77983525 | − | chr3 | 55654882 | 55655344 | + | chrX | 14703643 | 14704001 | + |
| chr16 | 79113212 | 79113451 | − | chr3 | 55705980 | 55706364 | + | chrX | 14996765 | 14997096 | + |
| chr16 | 79405844 | 79406276 | + | chr3 | 55790626 | 55790697 | + | chrX | 15016551 | 15016897 | − |
| chr16 | 79748893 | 79749002 | + | chr3 | 55815730 | 55816054 | − | chrX | 15027157 | 15027451 | + |
| chr16 | 79749134 | 79749303 | + | chr3 | 56446461 | 56446613 | − | chrX | 15096339 | 15096430 | − |
| chr16 | 79864904 | 79865261 | + | chr3 | 56446927 | 56447155 | − | chrX | 15096682 | 15097052 | − |
| chr16 | 79944019 | 79944245 | + | chr3 | 56455275 | 56455543 | + | chrX | 15127178 | 15127295 | + |
| chr16 | 79944453 | 79944500 | + | chr3 | 56894084 | 56894207 | + | chrX | 15128390 | 15128640 | + |
| chr16 | 80043363 | 80043627 | − | chr3 | 56898556 | 56898774 | − | chrX | 15138293 | 15138404 | − |
| chr16 | 80043642 | 80043774 | − | chr3 | 56899079 | 56899185 | − | chrX | 15138493 | 15138620 | − |
| chr16 | 80101566 | 80101947 | + | chr3 | 57983408 | 57983765 | + | chrX | 15406257 | 15406660 | − |
| chr16 | 80312997 | 80313423 | − | chr3 | 58633126 | 58633574 | + | chrX | 15456954 | 15457413 | − |
| chr16 | 80351713 | 80351918 | + | chr3 | 58639843 | 58639943 | + | chrX | 15565689 | 15566045 | + |
| chr16 | 80904150 | 80904216 | − | chr3 | 59862620 | 59862907 | − | chrX | 15887940 | 15888409 | − |
| chr16 | 80904527 | 80904840 | − | chr3 | 59929872 | 59930275 | − | chrX | 15997080 | 15997215 | + |
| chr16 | 81131907 | 81132096 | + | chr3 | 59984205 | 59984545 | − | chrX | 15997249 | 15997439 | + |
| chr16 | 81170245 | 81170609 | − | chr3 | 61253491 | 61253958 | − | chrX | 16040513 | 16040871 | − |
| chr16 | 81224265 | 81224325 | − | chr3 | 61307438 | 61307635 | − | chrX | 16105548 | 16106023 | + |
| chr16 | 81225198 | 81225463 | + | chr3 | 61779722 | 61779932 | + | chrX | 16140508 | 16140563 | + |
| chr16 | 81226518 | 81226577 | + | chr3 | 61779936 | 61780055 | + | chrX | 16140760 | 16140832 | + |
| chr16 | 81243782 | 81244228 | − | chr3 | 62390521 | 62390976 | + | chrX | 16186338 | 16186762 | − |
| chr16 | 81648836 | 81649083 | + | chr3 | 62955595 | 62955692 | − | chrX | 16335135 | 16335428 | − |
| chr16 | 81673096 | 81673233 | − | chr3 | 62955849 | 62956129 | − | chrX | 16374904 | 16375002 | − |
| chr16 | 81673544 | 81673721 | − | chr3 | 63049085 | 63049213 | + | chrX | 16375485 | 16375606 | − |
| chr16 | 81702035 | 81702488 | + | chr3 | 63049231 | 63049427 | + | chrX | 16375766 | 16376035 | − |
| chr16 | 81720407 | 81720728 | + | chr3 | 63063091 | 63063517 | − | chrX | 16387804 | 16388123 | + |
| chr16 | 81809154 | 81809598 | + | chr3 | 63142533 | 63142835 | − | chrX | 16388439 | 16388598 | + |
| chr16 | 82016377 | 82016818 | − | chr3 | 63298661 | 63298887 | − | chrX | 16404641 | 16405108 | + |
| chr16 | 82117616 | 82117910 | − | chr3 | 63432011 | 63432289 | + | chrX | 16489603 | 16489781 | + |
| chr16 | 82560841 | 82561308 | + | chr3 | 63486230 | 63486598 | + | chrX | 16506196 | 16506672 | + |
| chr16 | 82603554 | 82603668 | − | chr3 | 63489026 | 63489484 | − | chrX | 16545922 | 16546373 | − |
| chr16 | 82604113 | 82604433 | − | chr3 | 63621851 | 63622288 | − | chrX | 17198577 | 17199037 | + |
| chr16 | 82647350 | 82647786 | − | chr3 | 63672948 | 63673270 | − | chrX | 17206700 | 17207138 | + |
| chr16 | 82681627 | 82682046 | + | chr3 | 63704108 | 63704577 | − | chrX | 17241023 | 17241457 | + |
| chr16 | 82703275 | 82703596 | + | chr3 | 63778810 | 63778875 | − | chrX | 17282028 | 17282329 | + |
| chr16 | 82718001 | 82718297 | − | chr3 | 63778894 | 63779318 | − | chrX | 17297115 | 17297209 | + |
| chr16 | 82801851 | 82802218 | − | chr3 | 63806063 | 63806427 | + | chrX | 17298600 | 17298877 | + |
| chr16 | 82856749 | 82857005 | − | chr3 | 63808844 | 63808924 | + | chrX | 17327435 | 17327900 | + |
| chr16 | 82913704 | 82913978 | − | chr3 | 64210840 | 64211136 | − | chrX | 17338648 | 17338780 | − |
| chr16 | 82938623 | 82939103 | − | chr3 | 64730431 | 64730890 | − | chrX | 17340096 | 17340511 | − |
| chr16 | 82953975 | 82954374 | − | chr3 | 64924076 | 64924408 | − | chrX | 17378526 | 17378988 | − |
| chr16 | 82970998 | 82971410 | + | chr3 | 65329010 | 65329247 | + | chrX | 17382769 | 17383244 | + |
| chr16 | 83075018 | 83075288 | + | chr3 | 65340172 | 65340530 | − | chrX | 17476515 | 17476955 | − |
| chr16 | 83108810 | 83109077 | + | chr3 | 66029619 | 66029766 | + | chrX | 17511720 | 17512145 | + |
| chr16 | 83129352 | 83129417 | − | chr3 | 66126035 | 66126258 | − | chrX | 17534778 | 17535177 | + |
| chr16 | 83129611 | 83129844 | − | chr3 | 66126274 | 66126408 | − | chrX | 17538275 | 17538676 | − |
| chr16 | 83349459 | 83349581 | − | chr3 | 66596863 | 66596991 | + | chrX | 17557200 | 17557299 | + |
| chr16 | 83349900 | 83350148 | − | chr3 | 66597564 | 66597602 | + | chrX | 17559557 | 17560007 | + |
| chr16 | 83409026 | 83409469 | − | chr3 | 66780289 | 66780662 | + | chrX | 17562110 | 17562576 | − |
| chr16 | 83508534 | 83508796 | + | chr3 | 67082305 | 67082693 | − | chrX | 17579664 | 17580112 | + |
| chr16 | 83718049 | 83718265 | − | chr3 | 67116446 | 67116811 | + | chrX | 17668652 | 17668897 | − |
| chr16 | 83718856 | 83719040 | − | chr3 | 67218787 | 67219246 | − | chrX | 17814009 | 17814471 | − |
| chr16 | 83827228 | 83827462 | − | chr3 | 67225817 | 67226142 | − | chrX | 17836787 | 17837167 | − |
| chr16 | 84212182 | 84212285 | + | chr3 | 67297926 | 67298179 | − | chrX | 17864454 | 17864853 | − |
| chr16 | 84212672 | 84212777 | + | chr3 | 67639598 | 67639828 | + | chrX | 17880495 | 17880725 | − |
| chr16 | 84300152 | 84300504 | + | chr3 | 67672188 | 67672634 | + | chrX | 17881082 | 17881183 | − |
| chr16 | 84301301 | 84301312 | + | chr3 | 68039648 | 68040108 | − | chrX | 17914436 | 17914602 | + |
| chr16 | 84351167 | 84351507 | + | chr3 | 68050089 | 68050255 | − | chrX | 17914590 | 17914775 | + |
| chr16 | 84637326 | 84637682 | + | chr3 | 68054996 | 68055282 | − | chrX | 17948035 | 17948480 | + |
| chr16 | 84813334 | 84813660 | − | chr3 | 68056536 | 68056771 | − | chrX | 17975798 | 17976088 | + |
| chr16 | 84814855 | 84815264 | − | chr3 | 68552141 | 68552261 | + | chrX | 17976454 | 17976504 | + |
| chr16 | 84932901 | 84932971 | + | chr3 | 68552483 | 68552639 | + | chrX | 18022630 | 18023040 | + |
| chr16 | 84933085 | 84933144 | + | chr3 | 68598414 | 68598855 | − | chrX | 18136012 | 18136490 | + |
| chr16 | 84933446 | 84933672 | + | chr3 | 69462035 | 69462431 | − | chrX | 18697155 | 18697284 | − |
| chr16 | 85271040 | 85271336 | + | chr3 | 69525983 | 69526469 | + | chrX | 19063858 | 19064250 | − |
| chr16 | 85317663 | 85317858 | + | chr3 | 69528674 | 69529135 | − | chrX | 19105984 | 19106357 | − |
| chr16 | 85392063 | 85392178 | + | chr3 | 69601427 | 69601856 | + | chrX | 19163947 | 19164338 | + |
| chr16 | 85531108 | 85531222 | − | chr3 | 70484478 | 70484577 | − | chrX | 19174375 | 19174846 | − |
| chr16 | 85697824 | 85697989 | − | chr3 | 70570952 | 70571319 | − | chrX | 19329122 | 19329544 | + |
| chr16 | 85698135 | 85698225 | − | chr3 | 70655879 | 70656215 | + | chrX | 19330187 | 19330235 | + |
| chr16 | 85705768 | 85705855 | + | chr3 | 70672406 | 70672776 | − | chrX | 19444940 | 19445268 | + |
| chr16 | 85926440 | 85926536 | − | chr3 | 70894166 | 70894235 | − | chrX | 20015605 | 20015912 | + |
| chr16 | 85926623 | 85926765 | − | chr3 | 71631478 | 71631659 | + | chrX | 20313945 | 20314086 | + |
| chr16 | 85945875 | 85946053 | − | chr3 | 71631970 | 71632230 | + | chrX | 20317951 | 20318416 | + |
| chr16 | 86044406 | 86044585 | + | chr3 | 71843906 | 71843996 | − | chrX | 20326593 | 20327030 | + |
| chr16 | 86076629 | 86076671 | − | chr3 | 71861952 | 71862399 | + | chrX | 20340340 | 20340631 | − |
| chr16 | 86083266 | 86083695 | + | chr3 | 71893859 | 71894292 | + | chrX | 20343276 | 20343406 | − |
| chr16 | 86143862 | 86144298 | + | chr3 | 71942316 | 71942758 | − | chrX | 20345727 | 20346068 | − |
| chr16 | 86150081 | 86150545 | + | chr3 | 71956402 | 71956616 | − | chrX | 20490208 | 20490248 | + |
| chr16 | 86274108 | 86274467 | − | chr3 | 71985402 | 71985784 | + | chrX | 20490322 | 20490504 | + |
| chr16 | 86353782 | 86354200 | − | chr3 | 71992290 | 71992651 | + | chrX | 20491251 | 20491396 | + |
| chr16 | 86395468 | 86395802 | − | chr3 | 71993165 | 71993251 | + | chrX | 20508036 | 20508357 | + |
| chr16 | 86408486 | 86408944 | + | chr3 | 72011044 | 72011511 | + | chrX | 20532376 | 20532769 | + |
| chr16 | 86438292 | 86438555 | − | chr3 | 72048207 | 72048472 | − | chrX | 20599043 | 20599185 | − |
| chr16 | 86450659 | 86451010 | + | chr3 | 72061576 | 72062007 | − | chrX | 20599703 | 20599965 | − |
| chr16 | 86451090 | 86451153 | + | chr3 | 72102225 | 72102658 | − | chrX | 20741208 | 20741695 | − |
| chr16 | 86451199 | 86451386 | + | chr3 | 72116890 | 72117346 | + | chrX | 20795856 | 20795985 | − |
| chr16 | 86610659 | 86611006 | − | chr3 | 72128283 | 72128560 | + | chrX | 20796267 | 20796375 | − |
| chr16 | 86639708 | 86640092 | − | chr3 | 72146354 | 72146614 | − | chrX | 20796819 | 20796955 | − |
| chr16 | 86748596 | 86748814 | − | chr3 | 72170195 | 72170489 | + | chrX | 20802171 | 20802648 | + |
| chr16 | 86765618 | 86765920 | − | chr3 | 72173032 | 72173482 | + | chrX | 20892630 | 20892997 | + |
| chr16 | 86857240 | 86857610 | − | chr3 | 72176824 | 72177050 | − | chrX | 20953030 | 20953491 | − |
| chr16 | 86874221 | 86874519 | + | chr3 | 72182055 | 72182485 | + | chrX | 20994205 | 20994665 | + |
| chr16 | 86930130 | 86930424 | + | chr3 | 72188665 | 72189028 | + | chrX | 21009269 | 21009326 | + |
| chr16 | 87029274 | 87029499 | + | chr3 | 72189265 | 72189377 | + | chrX | 21009348 | 21009643 | + |
| chr16 | 87118133 | 87118375 | + | chr3 | 72217878 | 72218154 | − | chrX | 21077423 | 21077924 | − |
| chr16 | 87138139 | 87138297 | − | chr3 | 72218561 | 72218575 | − | chrX | 21145887 | 21146168 | + |
| chr16 | 87298877 | 87299189 | − | chr3 | 72223584 | 72223655 | − | chrX | 21158760 | 21158982 | + |
| chr16 | 87320294 | 87320444 | + | chr3 | 72232677 | 72232857 | + | chrX | 21172956 | 21173157 | − |
| chr16 | 87583668 | 87583796 | − | chr3 | 72233655 | 72233709 | + | chrX | 21664456 | 21664918 | + |
| chr16 | 87583883 | 87583953 | − | chr3 | 72259474 | 72259889 | + | chrX | 21699406 | 21699524 | + |
| chr16 | 88230372 | 88230485 | − | chr3 | 72287149 | 72287529 | − | chrX | 21699540 | 21699834 | + |
| chr16 | 89006354 | 89006591 | − | chr3 | 72314300 | 72314731 | + | chrX | 21759875 | 21760187 | + |
| chr16_GL383556v1_alt | 10529 | 10995 | − | chr3 | 72331835 | 72332250 | + | chrX | 21786339 | 21786730 | + |
| chr16_GL383556v1_alt | 33784 | 34250 | + | chr3 | 72574866 | 72575130 | − | chrX | 22137579 | 22137612 | + |
| chr16_GL383556v1_alt | 134672 | 134948 | + | chr3 | 72589509 | 72589920 | − | chrX | 22137777 | 22138187 | + |
| chr16_KI270853v1_alt | 340141 | 340240 | + | chr3 | 72627406 | 72627605 | − | chrX | 22213860 | 22213983 | − |
| chr16_KI270853v1_alt | 340709 | 340886 | + | chr3 | 72655943 | 72656116 | + | chrX | 22214484 | 22214808 | − |
| chr16_KI270853v1_alt | 341059 | 341274 | + | chr3 | 72656182 | 72656247 | + | chrX | 22294366 | 22294721 | + |
| chr16_KI270853v1_alt | 354487 | 354513 | − | chr3 | 72665079 | 72665418 | + | chrX | 22402892 | 22403252 | − |
| chr16_KI270853v1_alt | 355381 | 355737 | − | chr3 | 72699771 | 72699974 | − | chrX | 22408202 | 22408518 | + |
| chr16_KI270853v1_alt | 412929 | 413326 | + | chr3 | 72700486 | 72700517 | − | chrX | 22487103 | 22487498 | + |
| chr16_KI270853v1_alt | 413681 | 413718 | + | chr3 | 72700897 | 72700990 | − | chrX | 22521394 | 22521763 | − |
| chr16_KI270853v1_alt | 425180 | 425376 | − | chr3 | 72728010 | 72728232 | + | chrX | 22558716 | 22559067 | − |
| chr16_KI270853v1_alt | 425593 | 425804 | − | chr3 | 72987559 | 72987985 | + | chrX | 22641198 | 22641666 | + |
| chr16_KI270853v1_alt | 471977 | 472375 | − | chr3 | 73089277 | 73089482 | − | chrX | 22643572 | 22643949 | − |
| chr16_KI270853v1_alt | 979470 | 979677 | − | chr3 | 73109439 | 73109524 | − | chrX | 22746121 | 22746625 | + |
| chr16_KI270853v1_alt | 979850 | 980027 | − | chr3 | 73112495 | 73112898 | + | chrX | 22846313 | 22846784 | + |
| chr16_KI270853v1_alt | 980339 | 980439 | − | chr3 | 73912485 | 73912903 | − | chrX | 22950799 | 22951204 | + |
| chr16_KI270853v1_alt | 2095016 | 2095117 | + | chr3 | 74522452 | 74522887 | − | chrX | 22964390 | 22964688 | − |
| chr16_KI270853v1_alt | 2095429 | 2095606 | + | chr3 | 74692637 | 74692914 | − | chrX | 22968682 | 22969071 | − |
| chr16_KI270853v1_alt | 2095779 | 2095986 | + | chr3 | 74694444 | 74694824 | − | chrX | 22972865 | 22973142 | − |
| chr16_KI270853v1_alt | 2108833 | 2108936 | − | chr3 | 74748360 | 74748796 | − | chrX | 23080865 | 23081104 | − |
| chr16_KI270853v1_alt | 2109881 | 2110237 | − | chr3 | 74851448 | 74851746 | + | chrX | 23119386 | 23119567 | + |
| chr16_KI270853v1_alt | 2168158 | 2168555 | + | chr3 | 74892831 | 74892961 | − | chrX | 23139896 | 23140257 | + |
| chr16_KI270853v1_alt | 2168906 | 2168943 | + | chr3 | 74893272 | 74893576 | − | chrX | 23140778 | 23140825 | + |
| chr16_KI270853v1_alt | 2180407 | 2180603 | − | chr3 | 75495796 | 75496099 | − | chrX | 23194324 | 23194601 | + |
| chr16_KI270853v1_alt | 2180820 | 2181031 | − | chr3 | 75717901 | 75718215 | − | chrX | 23206361 | 23206694 | + |
| chr16_KI270853v1_alt | 2594698 | 2595138 | − | chr3 | 75909999 | 75910299 | − | chrX | 23311105 | 23311578 | + |
| chr17 | 402169 | 402418 | + | chr3 | 76440451 | 76440755 | − | chrX | 23324680 | 23325137 | − |
| chr17 | 414756 | 414884 | − | chr3 | 76850445 | 76850710 | + | chrX | 23341956 | 23342248 | − |
| chr17 | 415184 | 415320 | − | chr3 | 77673569 | 77673937 | + | chrX | 23365402 | 23365856 | − |
| chr17 | 2013333 | 2013571 | + | chr3 | 77674363 | 77674376 | + | chrX | 23371236 | 23371573 | − |
| chr17 | 2013842 | 2014102 | + | chr3 | 78821883 | 78822079 | − | chrX | 23373993 | 23374127 | − |
| chr17 | 5560460 | 5560872 | + | chr3 | 78822121 | 78822174 | − | chrX | 23380529 | 23380933 | − |
| chr17 | 5761282 | 5761566 | − | chr3 | 79537245 | 79537524 | − | chrX | 23408700 | 23409168 | − |
| chr17 | 6082196 | 6082415 | − | chr3 | 80023558 | 80023944 | + | chrX | 23514698 | 23515014 | + |
| chr17 | 6308114 | 6308246 | − | chr3 | 81096822 | 81097219 | − | chrX | 23515145 | 23515502 | + |
| chr17 | 6308703 | 6308934 | − | chr3 | 81097928 | 81098063 | − | chrX | 23515551 | 23515623 | + |
| chr17 | 8334126 | 8334340 | − | chr3 | 81212018 | 81212070 | − | chrX | 23515968 | 23516011 | + |
| chr17 | 9850320 | 9850683 | + | chr3 | 81250745 | 81251018 | + | chrX | 23555065 | 23555296 | + |
| chr17 | 9850994 | 9851074 | + | chr3 | 82033425 | 82033658 | + | chrX | 23555358 | 23555490 | + |
| chr17 | 11065615 | 11066050 | + | chr3 | 82033713 | 82033839 | + | chrX | 23937079 | 23937401 | − |
| chr17 | 13084397 | 13084596 | + | chr3 | 82036891 | 82037096 | + | chrX | 23937719 | 23937835 | − |
| chr17 | 13084625 | 13084685 | + | chr3 | 82037391 | 82037471 | + | chrX | 23953035 | 23953533 | + |
| chr17 | 13772258 | 13772646 | − | chr3 | 82196152 | 82196288 | + | chrX | 24315135 | 24315601 | − |
| chr17 | 14016423 | 14016534 | + | chr3 | 82717192 | 82717497 | + | chrX | 24360812 | 24361280 | + |
| chr17 | 14373786 | 14374201 | − | chr3 | 83577074 | 83577323 | − | chrX | 24392240 | 24392389 | − |
| chr17 | 14617450 | 14617743 | − | chr3 | 84367261 | 84367574 | + | chrX | 24392702 | 24392829 | − |
| chr17 | 14756813 | 14757023 | − | chr3 | 84695338 | 84695531 | − | chrX | 24428145 | 24428213 | + |
| chr17 | 14797764 | 14798229 | − | chr3 | 84696887 | 84697099 | − | chrX | 24428509 | 24428576 | + |
| chr17 | 15017693 | 15017888 | + | chr3 | 84805514 | 84805954 | + | chrX | 24428611 | 24428771 | + |
| chr17 | 15017905 | 15018057 | + | chr3 | 85467775 | 85467830 | − | chrX | 24451091 | 24451398 | + |
| chr17 | 15104493 | 15104850 | + | chr3 | 85468731 | 85469092 | − | chrX | 24653226 | 24653716 | + |
| chr17 | 15423044 | 15423370 | − | chr3 | 85933227 | 85933464 | + | chrX | 25514128 | 25514580 | − |
| chr17 | 16872483 | 16872759 | + | chr3 | 86335633 | 86335757 | + | chrX | 25965428 | 25965493 | − |
| chr17 | 17002739 | 17003014 | − | chr3 | 86336137 | 86336250 | + | chrX | 26013684 | 26014157 | − |
| chr17 | 17018275 | 17018441 | + | chr3 | 86379170 | 86379544 | − | chrX | 26413103 | 26413509 | + |
| chr17 | 17025316 | 17025470 | − | chr3 | 86915225 | 86915479 | + | chrX | 26823657 | 26823982 | − |
| chr17 | 17366576 | 17366947 | − | chr3 | 88026772 | 88027033 | − | chrX | 26823997 | 26824095 | − |
| chr17 | 17393141 | 17393392 | + | chr3 | 88668203 | 88668570 | + | chrX | 27196009 | 27196466 | − |
| chr17 | 17642881 | 17642940 | − | chr3 | 89058091 | 89058368 | + | chrX | 27914261 | 27914717 | − |
| chr17 | 18172944 | 18173309 | − | chr3 | 89375294 | 89375764 | − | chrX | 27925399 | 27925874 | + |
| chr17 | 18181133 | 18181176 | − | chr3 | 90024139 | 90024537 | − | chrX | 28549955 | 28550199 | + |
| chr17 | 18371797 | 18371949 | + | chr3 | 91351191 | 91351242 | − | chrX | 28550489 | 28550687 | + |
| chr17 | 18372251 | 18372315 | + | chr3 | 91351546 | 91351910 | − | chrX | 28561678 | 28561963 | − |
| chr17 | 19038244 | 19038524 | + | chr3 | 94060285 | 94060464 | + | chrX | 28707092 | 28707417 | + |
| chr17 | 19090668 | 19091092 | + | chr3 | 94608417 | 94608882 | + | chrX | 29151448 | 29151915 | − |
| chr17 | 19136022 | 19136302 | − | chr3 | 95114498 | 95114644 | + | chrX | 29302825 | 29303136 | − |
| chr17 | 19160993 | 19161417 | − | chr3 | 95340257 | 95340691 | − | chrX | 29530254 | 29530721 | − |
| chr17 | 19216837 | 19217261 | + | chr3 | 95717772 | 95718005 | − | chrX | 30153340 | 30153768 | + |
| chr17 | 21321419 | 21321531 | + | chr3 | 96474462 | 96474740 | + | chrX | 30504903 | 30505352 | + |
| chr17 | 21398514 | 21398717 | + | chr3 | 96555221 | 96555671 | + | chrX | 30521151 | 30521347 | − |
| chr17 | 21409390 | 21409788 | − | chr3 | 96960085 | 96960143 | − | chrX | 30521540 | 30521665 | − |
| chr17 | 21696855 | 21697251 | − | chr3 | 96961818 | 96962141 | − | chrX | 31094971 | 31095442 | − |
| chr17 | 21766387 | 21766551 | − | chr3 | 97190330 | 97190532 | + | chrX | 31550821 | 31550898 | − |
| chr17 | 27339036 | 27339159 | + | chr3 | 97306499 | 97306621 | + | chrX | 31551198 | 31551396 | − |
| chr17 | 27806012 | 27806332 | − | chr3 | 98117223 | 98117599 | + | chrX | 31572406 | 31572764 | + |
| chr17 | 27897349 | 27897453 | − | chr3 | 98447976 | 98448242 | − | chrX | 31679889 | 31680347 | + |
| chr17 | 27897765 | 27898086 | − | chr3 | 98448535 | 98448569 | − | chrX | 31685612 | 31686096 | + |
| chr17 | 27921321 | 27921692 | + | chr3 | 101960906 | 101961334 | + | chrX | 31812902 | 31813000 | − |
| chr17 | 27953945 | 27954155 | − | chr3 | 101961778 | 101961823 | + | chrX | 31813164 | 31813299 | − |
| chr17 | 27954322 | 27954418 | − | chr3 | 103475912 | 103476125 | + | chrX | 31813669 | 31813865 | − |
| chr17 | 28029176 | 28029618 | + | chr3 | 104604397 | 104604692 | − | chrX | 31907109 | 31907290 | − |
| chr17 | 30335352 | 30335429 | − | chr3 | 106204476 | 106204929 | + | chrX | 31908868 | 31908982 | − |
| chr17 | 30335736 | 30336111 | − | chr3 | 107322032 | 107322352 | + | chrX | 32210708 | 32210819 | + |
| chr17 | 30660468 | 30660589 | + | chr3 | 107468658 | 107469014 | − | chrX | 32215409 | 32215483 | + |
| chr17 | 30677573 | 30677954 | + | chr3 | 108146457 | 108146883 | − | chrX | 32233191 | 32233319 | + |
| chr17 | 32076492 | 32076613 | + | chr3 | 108614517 | 108614999 | + | chrX | 32268177 | 32268663 | + |
| chr17 | 32636558 | 32636948 | − | chr3 | 109768857 | 109768913 | − | chrX | 32307113 | 32307503 | − |
| chr17 | 33137364 | 33137799 | − | chr3 | 109769618 | 109769899 | − | chrX | 32449778 | 32450250 | − |
| chr17 | 33207600 | 33208037 | + | chr3 | 111012442 | 111012562 | + | chrX | 32520476 | 32520804 | + |
| chr17 | 33208876 | 33208980 | + | chr3 | 111013185 | 111013513 | + | chrX | 32612007 | 32612363 | − |
| chr17 | 33638740 | 33639021 | + | chr3 | 111581816 | 111582076 | − | chrX | 32742991 | 32743406 | + |
| chr17 | 33738462 | 33738701 | + | chr3 | 112507200 | 112507466 | − | chrX | 32967196 | 32967298 | − |
| chr17 | 33969428 | 33969881 | − | chr3 | 112507812 | 112508058 | + | chrX | 32967306 | 32967610 | − |
| chr17 | 34375086 | 34375214 | − | chr3 | 112508281 | 112508491 | − | chrX | 33324119 | 33324389 | − |
| chr17 | 34772056 | 34772406 | + | chr3 | 112751792 | 112752088 | − | chrX | 33539155 | 33539393 | + |
| chr17 | 34772907 | 34772947 | + | chr3 | 112752852 | 112753000 | − | chrX | 33539565 | 33539790 | + |
| chr17 | 34860535 | 34860957 | − | chr3 | 113812760 | 113813235 | − | chrX | 33735809 | 33735874 | − |
| chr17 | 35902163 | 35902216 | − | chr3 | 114932650 | 114932746 | − | chrX | 33830481 | 33830760 | − |
| chr17 | 35902513 | 35902575 | − | chr3 | 114933279 | 114933554 | − | chrX | 34245163 | 34245479 | + |
| chr17 | 36687775 | 36687887 | − | chr3 | 115468600 | 115468747 | − | chrX | 34634968 | 34635450 | + |
| chr17 | 37841409 | 37841543 | − | chr3 | 115470246 | 115470558 | − | chrX | 34680303 | 34680691 | − |
| chr17 | 40515097 | 40515546 | + | chr3 | 115852752 | 115853032 | − | chrX | 35021871 | 35022336 | − |
| chr17 | 41159153 | 41159558 | − | chr3 | 116534835 | 116535125 | − | chrX | 35429301 | 35429614 | + |
| chr17 | 41176913 | 41177352 | − | chr3 | 117119272 | 117119682 | + | chrX | 35653913 | 35653997 | + |
| chr17 | 41229690 | 41230078 | − | chr3 | 117881322 | 117881549 | + | chrX | 36103132 | 36103506 | − |
| chr17 | 41235176 | 41235562 | − | chr3 | 117881666 | 117881865 | + | chrX | 36225826 | 36226151 | − |
| chr17 | 41246858 | 41247231 | − | chr3 | 117917645 | 117918084 | + | chrX | 36315680 | 36315916 | + |
| chr17 | 41252392 | 41252670 | − | chr3 | 119588189 | 119588637 | − | chrX | 36382033 | 36382368 | − |
| chr17 | 41262567 | 41262818 | − | chr3 | 119611994 | 119612303 | + | chrX | 36392731 | 36393025 | − |
| chr17 | 41268415 | 41268688 | − | chr3 | 120398290 | 120398744 | + | chrX | 36479329 | 36479690 | − |
| chr17 | 41273351 | 41273465 | − | chr3 | 120569350 | 120569822 | + | chrX | 36791680 | 36791923 | + |
| chr17 | 41273683 | 41273743 | − | chr3 | 120625228 | 120625602 | − | chrX | 36792412 | 36792605 | + |
| chr17 | 41277756 | 41278172 | − | chr3 | 121007649 | 121007834 | − | chrX | 37035950 | 37036018 | − |
| chr17 | 41720678 | 41720795 | + | chr3 | 122470967 | 122471209 | − | chrX | 37036384 | 37036807 | − |
| chr17 | 41721097 | 41721193 | + | chr3 | 122556568 | 122556821 | − | chrX | 37039495 | 37039895 | + |
| chr17 | 42189057 | 42189426 | − | chr3 | 122604353 | 122604425 | − | chrX | 37050371 | 37050545 | + |
| chr17 | 42820056 | 42820290 | + | chr3 | 122605125 | 122605369 | − | chrX | 37515835 | 37516235 | − |
| chr17 | 43684835 | 43685172 | − | chr3 | 123158366 | 123158639 | − | chrX | 37518957 | 37519353 | + |
| chr17 | 43822728 | 43823185 | + | chr3 | 123436969 | 123437462 | − | chrX | 37519719 | 37519790 | + |
| chr17 | 44299508 | 44299701 | − | chr3 | 123966213 | 123966580 | + | chrX | 37610856 | 37611222 | − |
| chr17 | 44300032 | 44300328 | − | chr3 | 124014191 | 124014658 | + | chrX | 37656870 | 37657338 | − |
| chr17 | 45729823 | 45730222 | + | chr3 | 124323832 | 124324274 | + | chrX | 37727098 | 37727582 | − |
| chr17 | 45766219 | 45766427 | + | chr3 | 125843340 | 125843642 | + | chrX | 37789854 | 37790093 | + |
| chr17 | 45836557 | 45836815 | − | chr3 | 125985572 | 125985939 | − | chrX | 37790149 | 37790324 | + |
| chr17 | 49087642 | 49088074 | + | chr3 | 126546143 | 126546378 | + | chrX | 37941440 | 37941715 | + |
| chr17 | 51570779 | 51571100 | + | chr3 | 126546760 | 126546904 | + | chrX | 37974968 | 37975306 | − |
| chr17 | 51776902 | 51776963 | − | chr3 | 126556260 | 126556526 | + | chrX | 37987654 | 37987840 | + |
| chr17 | 52203886 | 52204295 | + | chr3 | 127072552 | 127072760 | + | chrX | 37987886 | 37987972 | + |
| chr17 | 52692373 | 52692777 | − | chr3 | 127093670 | 127093766 | + | chrX | 38081516 | 38081988 | − |
| chr17 | 54092395 | 54092649 | − | chr3 | 127095920 | 127096101 | + | chrX | 38113740 | 38114068 | + |
| chr17 | 54848558 | 54848945 | − | chr3 | 127465663 | 127465814 | − | chrX | 38124570 | 38125060 | − |
| chr17 | 55883477 | 55883715 | + | chr3 | 127975002 | 127975280 | − | chrX | 38157119 | 38157204 | − |
| chr17 | 55884008 | 55884228 | + | chr3 | 129983805 | 129984222 | − | chrX | 38157720 | 38158085 | − |
| chr17 | 55886269 | 55886722 | + | chr3 | 130172465 | 130172773 | − | chrX | 38206601 | 38206794 | − |
| chr17 | 56248778 | 56248955 | − | chr3 | 130173079 | 130173188 | − | chrX | 38349100 | 38349326 | − |
| chr17 | 62333701 | 62334141 | − | chr3 | 130213018 | 130213430 | − | chrX | 38349713 | 38349746 | − |
| chr17 | 62865512 | 62865983 | − | chr3 | 130509279 | 130509490 | + | chrX | 38451877 | 38452369 | − |
| chr17 | 62866659 | 62866929 | + | chr3 | 130521587 | 130522040 | + | chrX | 38474374 | 38474756 | − |
| chr17 | 62889739 | 62889844 | + | chr3 | 130824661 | 130825115 | + | chrX | 38491468 | 38491942 | − |
| chr17 | 62895763 | 62896175 | + | chr3 | 131073747 | 131074212 | + | chrX | 38510687 | 38510830 | + |
| chr17 | 62896789 | 62896823 | + | chr3 | 131428644 | 131429088 | + | chrX | 38511188 | 38511267 | + |
| chr17 | 63995558 | 63995849 | − | chr3 | 131553470 | 131553739 | + | chrX | 38511579 | 38511708 | + |
| chr17 | 65102175 | 65102520 | + | chr3 | 131586258 | 131586684 | − | chrX | 38757111 | 38757362 | − |
| chr17 | 65258255 | 65258681 | + | chr3 | 131704185 | 131704636 | + | chrX | 38759673 | 38759794 | − |
| chr17 | 65416585 | 65416881 | − | chr3 | 131720092 | 131720284 | + | chrX | 38759856 | 38760024 | − |
| chr17 | 65587315 | 65587662 | − | chr3 | 131720583 | 131720864 | + | chrX | 38924473 | 38924979 | + |
| chr17 | 66725845 | 66726045 | − | chr3 | 131984584 | 131984969 | + | chrX | 38955998 | 38956449 | + |
| chr17 | 69110245 | 69110705 | + | chr3 | 131985319 | 131985385 | + | chrX | 38989408 | 38989724 | − |
| chr17 | 69651015 | 69651336 | + | chr3 | 132058466 | 132058812 | + | chrX | 39049244 | 39049721 | + |
| chr17 | 69706835 | 69707086 | − | chr3 | 132193822 | 132194274 | − | chrX | 39121983 | 39122445 | − |
| chr17 | 70258307 | 70258595 | − | chr3 | 132222705 | 132223174 | − | chrX | 39211558 | 39211759 | − |
| chr17 | 70422146 | 70422512 | − | chr3 | 133243843 | 133244125 | − | chrX | 39212176 | 39212198 | − |
| chr17 | 71508473 | 71508617 | + | chr3 | 133453392 | 133453799 | − | chrX | 39212527 | 39212759 | − |
| chr17 | 71509135 | 71509599 | + | chr3 | 133668647 | 133668995 | + | chrX | 39229784 | 39230248 | − |
| chr17 | 73388257 | 73388486 | + | chr3 | 133669302 | 133669401 | + | chrX | 39305698 | 39305987 | − |
| chr17 | 73388693 | 73388712 | + | chr3 | 134774803 | 134775165 | + | chrX | 39308337 | 39308801 | − |
| chr17 | 73720452 | 73720843 | + | chr3 | 134775604 | 134775666 | + | chrX | 39339183 | 39339484 | + |
| chr17 | 73745183 | 73745379 | − | chr3 | 135177362 | 135177761 | + | chrX | 39341600 | 39341774 | + |
| chr17 | 73790962 | 73791459 | − | chr3 | 135715226 | 135715664 | + | chrX | 39341803 | 39342013 | + |
| chr17 | 73942087 | 73942232 | − | chr3 | 136559273 | 136559574 | − | chrX | 39368842 | 39368905 | − |
| chr17 | 73942293 | 73942476 | − | chr3 | 136801662 | 136802069 | − | chrX | 39369197 | 39369586 | − |
| chr17 | 73973968 | 73974138 | + | chr3 | 137236052 | 137236397 | + | chrX | 39390905 | 39391000 | − |
| chr17 | 73974273 | 73974342 | + | chr3 | 137348049 | 137348430 | + | chrX | 39391477 | 39391860 | − |
| chr17 | 74120560 | 74120969 | − | chr3 | 139160942 | 139161021 | + | chrX | 39395441 | 39395911 | − |
| chr17 | 74952657 | 74952787 | − | chr3 | 139670214 | 139670540 | + | chrX | 39409936 | 39410235 | − |
| chr17 | 76865615 | 76866063 | − | chr3 | 139680342 | 139680573 | − | chrX | 39439436 | 39439645 | − |
| chr17 | 76880680 | 76880942 | + | chr3 | 139953441 | 139953894 | + | chrX | 39439876 | 39440086 | − |
| chr17 | 76941702 | 76942037 | − | chr3 | 140754181 | 140754631 | − | chrX | 39479283 | 39479663 | + |
| chr17 | 77393065 | 77393329 | − | chr3 | 141094790 | 141094880 | + | chrX | 39480767 | 39481231 | − |
| chr17 | 77579389 | 77579769 | + | chr3 | 141182028 | 141182382 | + | chrX | 39496809 | 39497008 | − |
| chr17 | 77798471 | 77798653 | − | chr3 | 143919191 | 143919562 | + | chrX | 39497560 | 39497814 | − |
| chr17 | 77831066 | 77831476 | + | chr3 | 144483406 | 144483483 | + | chrX | 39508098 | 39508558 | + |
| chr17 | 79107800 | 79108111 | + | chr3 | 144991552 | 144991714 | + | chrX | 39525803 | 39526210 | + |
| chr17 | 79505322 | 79505596 | + | chr3 | 145161077 | 145161289 | + | chrX | 39534952 | 39535374 | + |
| chr17 | 79650855 | 79651270 | + | chr3 | 145161303 | 145161515 | + | chrX | 39559882 | 39560326 | + |
| chr17 | 79675037 | 79675453 | + | chr3 | 145853608 | 145853732 | + | chrX | 39567955 | 39568165 | − |
| chr17_GL000258v2_alt | 951347 | 951605 | + | chr3 | 146164092 | 146164264 | − | chrX | 39568477 | 39568593 | − |
| chr17_GL000258v2_alt | 1021783 | 1021991 | − | chr3 | 147889533 | 147889958 | − | chrX | 39570378 | 39570821 | + |
| chr17_GL000258v2_alt | 1057843 | 1058242 | − | chr3 | 148282169 | 148282515 | + | chrX | 39571320 | 39571635 | + |
| chr17_GL383563v3_alt | 375223 | 375472 | + | chr3 | 148469789 | 148470156 | − | chrX | 39625644 | 39625761 | + |
| chr17_GL383563v3_alt | 121922 | 122288 | − | chr3 | 148474998 | 148475196 | − | chrX | 39626937 | 39627298 | + |
| chr17_JH159147v1_alt | 28935 | 29354 | + | chr3 | 148475590 | 148475860 | − | chrX | 39631681 | 39632141 | − |
| chr17_JH159147v1_alt | 33899 | 33944 | + | chr3 | 148832422 | 148832890 | − | chrX | 39672361 | 39672812 | + |
| chr17_JH159147v1_alt | 34019 | 34270 | + | chr3 | 149405394 | 149405531 | − | chrX | 39694270 | 39694691 | + |
| chr17_JH159147v1_alt | 44187 | 44465 | + | chr3 | 150114038 | 150114266 | + | chrX | 39697580 | 39697630 | + |
| chr17_JH159147v1_alt | 49625 | 49998 | + | chr3 | 150223253 | 150223544 | + | chrX | 39700240 | 39700499 | − |
| chr17_JH159147v1_alt | 61227 | 61600 | + | chr3 | 152179337 | 152179717 | + | chrX | 39700520 | 39700647 | − |
| chr17_JH159147v1_alt | 66709 | 67097 | + | chr3 | 152868515 | 152868718 | + | chrX | 39744262 | 39744685 | − |
| chr17_JH159148v1_alt | 28935 | 29354 | + | chr3 | 152868778 | 152868872 | + | chrX | 39761265 | 39761548 | − |
| chr17_JH159148v1_alt | 33367 | 33429 | + | chr3 | 153252772 | 153252819 | + | chrX | 39769431 | 39769754 | − |
| chr17_JH159148v1_alt | 33645 | 33759 | + | chr3 | 153254031 | 153254137 | + | chrX | 39778228 | 39778461 | + |
| chr17_JH159148v1_alt | 38422 | 38695 | + | chr3 | 153254437 | 153254594 | + | chrX | 39810357 | 39810763 | − |
| chr17_JH159148v1_alt | 44292 | 44543 | + | chr3 | 153855901 | 153856237 | + | chrX | 39851967 | 39852239 | − |
| chr17_JH159148v1_alt | 54440 | 54718 | + | chr3 | 154495325 | 154495476 | + | chrX | 39881330 | 39881621 | − |
| chr17_JH159148v1_alt | 59883 | 60256 | + | chr3 | 154495472 | 154495593 | + | chrX | 39925767 | 39925839 | − |
| chr17_KI270857v1_alt | 136695 | 136748 | − | chr3 | 155449729 | 155450194 | + | chrX | 39926244 | 39926441 | − |
| chr17_KI270857v1_alt | 137045 | 137107 | − | chr3 | 156240767 | 156241172 | − | chrX | 39926743 | 39926826 | − |
| chr17_KI270857v1_alt | 923291 | 923403 | − | chr3 | 156933123 | 156933180 | + | chrX | 39949390 | 39949434 | − |
| chr17_KI270857v1_alt | 2080098 | 2080232 | − | chr3 | 157404455 | 157404572 | − | chrX | 39949439 | 39949678 | − |
| chr17_KI270907v1_alt | 220 | 469 | − | chr3 | 157404606 | 157404793 | − | chrX | 39982868 | 39982922 | − |
| chr17_KI270908v1_alt | 431919 | 432318 | + | chr3 | 157609142 | 157609466 | − | chrX | 39984200 | 39984401 | − |
| chr17_KI270908v1_alt | 468332 | 468540 | + | chr3 | 159150131 | 159150455 | − | chrX | 39989413 | 39989531 | + |
| chr17_KI270908v1_alt | 538679 | 538937 | − | chr3 | 159331028 | 159331473 | − | chrX | 39989909 | 39990064 | + |
| chr18 | 854082 | 854276 | − | chr3 | 159444010 | 159444317 | − | chrX | 40186856 | 40187125 | − |
| chr18 | 854525 | 854700 | − | chr3 | 161458820 | 161459048 | + | chrX | 40187323 | 40187446 | − |
| chr18 | 1095588 | 1095832 | − | chr3 | 161732446 | 161732799 | − | chrX | 40304187 | 40304504 | − |
| chr18 | 1984898 | 1985238 | + | chr3 | 163522890 | 163523244 | − | chrX | 40312342 | 40312426 | − |
| chr18 | 5067902 | 5068133 | + | chr3 | 163651204 | 163651407 | + | chrX | 40312815 | 40313125 | − |
| chr18 | 5139243 | 5139662 | − | chr3 | 164812884 | 164813244 | − | chrX | 40324113 | 40324412 | + |
| chr18 | 5704772 | 5704918 | − | chr3 | 166287060 | 166287258 | + | chrX | 40324716 | 40324766 | + |
| chr18 | 5704999 | 5705115 | − | chr3 | 167125712 | 167126163 | − | chrX | 40411811 | 40412156 | + |
| chr18 | 6510683 | 6511048 | − | chr3 | 167199063 | 167199319 | − | chrX | 40414196 | 40414651 | − |
| chr18 | 6613808 | 6614270 | − | chr3 | 168307584 | 168307723 | + | chrX | 40480657 | 40480984 | − |
| chr18 | 7385477 | 7385699 | − | chr3 | 169438343 | 169438693 | − | chrX | 40488936 | 40489390 | + |
| chr18 | 7385751 | 7385856 | − | chr3 | 169697094 | 169697489 | + | chrX | 40507578 | 40507752 | − |
| chr18 | 7565205 | 7565434 | + | chr3 | 169915010 | 169915396 | + | chrX | 40509519 | 40509899 | − |
| chr18 | 7723617 | 7723911 | + | chr3 | 170015531 | 170015717 | + | chrX | 40521631 | 40521942 | + |
| chr18 | 8515895 | 8516060 | − | chr3 | 170016634 | 170016760 | + | chrX | 40528725 | 40528839 | + |
| chr18 | 8516673 | 8516858 | − | chr3 | 170608574 | 170609049 | + | chrX | 40530477 | 40530635 | + |
| chr18 | 9873093 | 9873216 | + | chr3 | 171585126 | 171585315 | + | chrX | 40539909 | 40540135 | + |
| chr18 | 10165272 | 10165513 | − | chr3 | 171586923 | 171587119 | + | chrX | 40783166 | 40783250 | + |
| chr18 | 10358188 | 10358236 | − | chr3 | 171594045 | 171594238 | + | chrX | 40784048 | 40784307 | + |
| chr18 | 11416612 | 11417081 | − | chr3 | 171721207 | 171721579 | − | chrX | 40789578 | 40789605 | − |
| chr18 | 12894906 | 12895299 | − | chr3 | 172294122 | 172294246 | − | chrX | 40789972 | 40790077 | − |
| chr18 | 14339574 | 14339890 | + | chr3 | 172294623 | 172294741 | − | chrX | 40790431 | 40790506 | − |
| chr18 | 15180417 | 15180550 | + | chr3 | 174361318 | 174361392 | + | chrX | 40879111 | 40879180 | − |
| chr18 | 21669861 | 21670317 | − | chr3 | 175197727 | 175197917 | − | chrX | 40879533 | 40879899 | − |
| chr18 | 21915241 | 21915322 | − | chr3 | 175726363 | 175726760 | + | chrX | 40930562 | 40930717 | − |
| chr18 | 21915451 | 21915742 | − | chr3 | 176954958 | 176955142 | − | chrX | 40930959 | 40931088 | − |
| chr18 | 22340412 | 22340553 | − | chr3 | 176955424 | 176955589 | − | chrX | 40931122 | 40931327 | − |
| chr18 | 22472960 | 22473337 | − | chr3 | 176955879 | 176956043 | − | chrX | 41394074 | 41394305 | + |
| chr18 | 23777897 | 23778030 | − | chr3 | 177089393 | 177089561 | + | chrX | 41944259 | 41944691 | − |
| chr18 | 23960804 | 23960896 | + | chr3 | 178191119 | 178191606 | + | chrX | 41945697 | 41946073 | + |
| chr18 | 23961034 | 23961239 | + | chr3 | 178645393 | 178645815 | − | chrX | 41970590 | 41970904 | + |
| chr18 | 25004578 | 25004955 | + | chr3 | 178854611 | 178854923 | − | chrX | 41971607 | 41971667 | + |
| chr18 | 25542340 | 25542764 | − | chr3 | 179707234 | 179707669 | + | chrX | 42000213 | 42000475 | − |
| chr18 | 26170060 | 26170241 | + | chr3 | 180288271 | 180288536 | + | chrX | 42072499 | 42072800 | − |
| chr18 | 26928504 | 26928927 | − | chr3 | 180297290 | 180297436 | + | chrX | 42082215 | 42082456 | − |
| chr18 | 27210505 | 27210841 | + | chr3 | 180347989 | 180348381 | + | chrX | 42102753 | 42103119 | + |
| chr18 | 27211136 | 27211245 | + | chr3 | 183449426 | 183449733 | − | chrX | 42195365 | 42195837 | − |
| chr18 | 27214403 | 27214525 | + | chr3 | 184797569 | 184797604 | + | chrX | 42210180 | 42210355 | + |
| chr18 | 27643541 | 27643981 | + | chr3 | 184798141 | 184798566 | + | chrX | 42210676 | 42210913 | + |
| chr18 | 28614559 | 28614798 | + | chr3 | 185866056 | 185866085 | − | chrX | 42368708 | 42368756 | + |
| chr18 | 31363886 | 31364336 | − | chr3 | 185866381 | 185866801 | − | chrX | 42369219 | 42369678 | + |
| chr18 | 32145449 | 32145566 | + | chr3 | 186526645 | 186526984 | − | chrX | 42382914 | 42383248 | + |
| chr18 | 32146107 | 32146145 | + | chr3 | 187404305 | 187404394 | + | chrX | 42388837 | 42389283 | − |
| chr18 | 32146410 | 32146501 | + | chr3 | 187405364 | 187405817 | + | chrX | 42395895 | 42396384 | − |
| chr18 | 34372730 | 34373174 | − | chr3 | 190139940 | 190140148 | + | chrX | 42484081 | 42484541 | + |
| chr18 | 34957244 | 34957570 | + | chr3 | 190699069 | 190699414 | + | chrX | 42521216 | 42521655 | − |
| chr18 | 34957899 | 34958192 | + | chr3 | 190945101 | 190945226 | + | chrX | 42635700 | 42636048 | − |
| chr18 | 34958211 | 34958265 | + | chr3 | 191304281 | 191304557 | + | chrX | 42643721 | 42644146 | − |
| chr18 | 37474783 | 37475253 | + | chr3 | 191754952 | 191754986 | − | chrX | 42695749 | 42696206 | − |
| chr18 | 38208573 | 38208802 | + | chr3 | 191755298 | 191755543 | − | chrX | 42752521 | 42752969 | + |
| chr18 | 38318898 | 38319339 | − | chr3 | 194044962 | 194045399 | + | chrX | 42795601 | 42795935 | − |
| chr18 | 39944090 | 39944309 | − | chr3 | 194770738 | 194771032 | + | chrX | 42796706 | 42796854 | − |
| chr18 | 39944939 | 39945309 | − | chr3 | 196292375 | 196292424 | + | chrX | 42845425 | 42845634 | + |
| chr18 | 39994347 | 39994789 | − | chr3 | 196292722 | 196292858 | + | chrX | 42845958 | 42846195 | + |
| chr18 | 40389024 | 40389385 | + | chr3_JH636055v2_alt | 10797 | 10944 | + | chrX | 42955377 | 42955513 | + |
| chr18 | 40389658 | 40389748 | + | chr3_JH636055v2_alt | 11207 | 11379 | + | chrX | 43071802 | 43072095 | + |
| chr18 | 40636363 | 40636645 | + | chr3_KI270780v1_alt | 183646 | 183948 | + | chrX | 43145600 | 43145747 | + |
| chr18 | 40636690 | 40636768 | + | chr4 | 623140 | 623396 | − | chrX | 43145753 | 43145809 | + |
| chr18 | 40980135 | 40980387 | − | chr4 | 3831018 | 3831194 | − | chrX | 43146426 | 43146565 | + |
| chr18 | 41004195 | 41004548 | − | chr4 | 4025934 | 4026227 | − | chrX | 43462586 | 43462796 | − |
| chr18 | 42197538 | 42197959 | + | chr4 | 4254301 | 4254406 | + | chrX | 43462993 | 43463265 | − |
| chr18 | 42355036 | 42355492 | − | chr4 | 4693523 | 4693896 | − | chrX | 43513517 | 43513667 | + |
| chr18 | 42585117 | 42585504 | + | chr4 | 4716847 | 4716965 | + | chrX | 43514477 | 43514920 | + |
| chr18 | 45270068 | 45270354 | + | chr4 | 4718236 | 4718510 | + | chrX | 43549750 | 43550156 | + |
| chr18 | 45450748 | 45451171 | − | chr4 | 4733682 | 4734074 | − | chrX | 43866867 | 43867171 | − |
| chr18 | 45982238 | 45982497 | + | chr4 | 4924069 | 4924439 | − | chrX | 44117374 | 44117836 | − |
| chr18 | 45982795 | 45983004 | + | chr4 | 5024610 | 5024818 | + | chrX | 44154107 | 44154419 | + |
| chr18 | 46314213 | 46314426 | + | chr4 | 5025494 | 5025771 | + | chrX | 44154729 | 44154894 | + |
| chr18 | 46314952 | 46315099 | + | chr4 | 5111799 | 5112223 | − | chrX | 45009669 | 45009951 | + |
| chr18 | 47276067 | 47276484 | + | chr4 | 5152406 | 5152617 | − | chrX | 45234112 | 45234590 | − |
| chr18 | 47843059 | 47843402 | + | chr4 | 5167477 | 5167928 | − | chrX | 45386983 | 45387181 | + |
| chr18 | 47944677 | 47944831 | + | chr4 | 5265206 | 5265411 | − | chrX | 45887723 | 45888005 | − |
| chr18 | 48424239 | 48424311 | − | chr4 | 5471922 | 5472127 | − | chrX | 46130304 | 46130581 | + |
| chr18 | 48425234 | 48425540 | + | chr4 | 5664690 | 5664890 | + | chrX | 46130621 | 46130831 | + |
| chr18 | 50189191 | 50189644 | − | chr4 | 5826270 | 5826402 | − | chrX | 46270437 | 46270898 | + |
| chr18 | 50725451 | 50725715 | + | chr4 | 5840015 | 5840363 | − | chrX | 46292710 | 46293092 | + |
| chr18 | 50734336 | 50734660 | − | chr4 | 6230623 | 6230727 | + | chrX | 69430789 | 69431018 | − |
| chr18 | 50734974 | 50735070 | − | chr4 | 6244531 | 6244611 | − | chrX | 80944394 | 80944840 | + |
| chr18 | 51259945 | 51260417 | − | chr4 | 6245180 | 6245347 | − | chrX | 81476062 | 81476453 | − |
| chr18 | 51737652 | 51738060 | − | chr4 | 6440794 | 6441160 | − | chrX | 87039138 | 87039404 | − |
| chr18 | 53154008 | 53154396 | + | chr4 | 6746160 | 6746256 | − | chrX | 87039426 | 87039490 | − |
| chr18 | 53854489 | 53854861 | − | chr4 | 7110593 | 7110729 | + | chrX | 87192193 | 87192613 | − |
| chr18 | 53862980 | 53863206 | − | chr4 | 7122537 | 7122706 | − | chrX | 88922040 | 88922104 | − |
| chr18 | 53935643 | 53935979 | + | chr4 | 7122704 | 7122775 | − | chrX | 88922422 | 88922774 | − |
| chr18 | 54506597 | 54506792 | − | chr4 | 7607949 | 7608018 | − | chrX | 89257930 | 89258035 | − |
| chr18 | 56282661 | 56283076 | + | chr4 | 7608088 | 7608188 | − | chrX | 89796103 | 89796354 | + |
| chr18 | 56303577 | 56304045 | + | chr4 | 8316931 | 8317305 | − | chrX | 89950725 | 89950952 | + |
| chr18 | 56394032 | 56394456 | + | chr4 | 8325595 | 8325882 | + | chrX | 94309182 | 94309619 | − |
| chr18 | 56434184 | 56434436 | + | chr4 | 8735505 | 8735829 | − | chrX | 94349542 | 94349794 | + |
| chr18 | 57168143 | 57168615 | − | chr4 | 8835980 | 8836352 | + | chrX | 98388575 | 98388910 | + |
| chr18 | 57290534 | 57290833 | − | chr4 | 9610796 | 9610905 | + | chrX | 100199933 | 100200254 | − |
| chr18 | 57461452 | 57461930 | − | chr4 | 9611206 | 9611511 | + | chrX | 102157744 | 102157968 | − |
| chr18 | 57920924 | 57921282 | + | chr4 | 9842830 | 9843255 | − | chrX | 107345000 | 107345458 | − |
| chr18 | 57956529 | 57956589 | − | chr4 | 9857657 | 9857849 | + | chrX | 112040801 | 112041245 | − |
| chr18 | 57956647 | 57957044 | − | chr4 | 9924356 | 9924555 | + | chrX | 113675257 | 113675509 | − |
| chr18 | 58465294 | 58465433 | + | chr4 | 9924631 | 9924750 | + | chrX | 116791358 | 116791568 | − |
| chr18 | 58465573 | 58465678 | + | chr4 | 10756547 | 10757003 | − | chrX | 116792096 | 116792126 | − |
| chr18 | 58502677 | 58503132 | + | chr4 | 10890338 | 10890566 | + | chrX | 120059308 | 120059470 | + |
| chr18 | 58776896 | 58777133 | + | chr4 | 10902711 | 10903167 | − | chrX | 120176883 | 120177045 | − |
| chr18 | 59518680 | 59519120 | + | chr4 | 11002640 | 11002831 | + | chrX | 122049383 | 122049659 | + |
| chr18 | 60676697 | 60677080 | − | chr4 | 11003261 | 11003287 | + | chrX | 122049690 | 122049886 | + |
| chr18 | 60693306 | 60693559 | − | chr4 | 11003672 | 11003770 | + | chrX | 123344248 | 123344674 | + |
| chr18 | 60840114 | 60840370 | − | chr4 | 11189065 | 11189426 | − | chrX | 123754054 | 123754391 | + |
| chr18 | 60894650 | 60894827 | + | chr4 | 11204583 | 11204646 | − | chrX | 123755005 | 123755164 | + |
| chr18 | 61927061 | 61927519 | − | chr4 | 11584002 | 11584236 | − | chrX | 127678252 | 127678477 | − |
| chr18 | 63019853 | 63020316 | − | chr4 | 11685025 | 11685481 | − | chrX | 128663267 | 128663404 | + |
| chr18 | 63136479 | 63136925 | − | chr4 | 11927828 | 11927932 | − | chrX | 128999442 | 128999916 | − |
| chr18 | 64276813 | 64277095 | − | chr4 | 11928786 | 11929067 | − | chrX | 129443038 | 129443483 | − |
| chr18 | 64332126 | 64332522 | + | chr4 | 11954684 | 11954844 | + | chrX | 129697169 | 129697461 | − |
| chr18 | 64826351 | 64826823 | − | chr4 | 12045001 | 12045393 | + | chrX | 131983362 | 131983506 | + |
| chr18 | 65113524 | 65113794 | − | chr4 | 12199026 | 12199462 | − | chrX | 131983722 | 131983972 | + |
| chr18 | 65479289 | 65479442 | + | chr4 | 12216817 | 12217173 | + | chrX | 135124160 | 135124555 | − |
| chr18 | 65479456 | 65479683 | + | chr4 | 12223224 | 12223497 | − | chrX | 135247987 | 135248382 | + |
| chr18 | 66143140 | 66143531 | + | chr4 | 12406434 | 12406639 | + | chrX | 135304923 | 135305166 | + |
| chr18 | 66304763 | 66305128 | + | chr4 | 12411563 | 12412007 | + | chrX | 135305160 | 135305357 | + |
| chr18 | 66812569 | 66812871 | + | chr4 | 12452318 | 12452507 | + | chrX | 135390913 | 135391303 | + |
| chr18 | 67686041 | 67686260 | + | chr4 | 12454470 | 12454906 | + | chrX | 137955715 | 137956071 | + |
| chr18 | 68182569 | 68182773 | + | chr4 | 12624903 | 12625194 | + | chrX | 138914956 | 138915322 | + |
| chr18 | 68652625 | 68652953 | + | chr4 | 12628408 | 12628527 | + | chrX | 140425483 | 140425738 | − |
| chr18 | 69353333 | 69353624 | + | chr4 | 13046807 | 13047055 | + | chrX | 141922773 | 141923108 | + |
| chr18 | 69357055 | 69357380 | + | chr4 | 13047405 | 13047487 | + | chrX | 144901591 | 144901960 | − |
| chr18 | 70958680 | 70959161 | + | chr4 | 13118122 | 13118238 | + | chrX | 147422589 | 147422937 | + |
| chr18 | 71554722 | 71554986 | − | chr4 | 13654059 | 13654130 | − | chrX | 149464855 | 149465135 | + |
| chr18 | 72332752 | 72332896 | − | chr4 | 13655010 | 13655406 | − | chrX | 149465532 | 149465669 | + |
| chr18 | 72878128 | 72878294 | + | chr4 | 14096868 | 14097269 | − | chrX | 149466823 | 149466892 | + |
| chr18 | 72878326 | 72878499 | + | chr4 | 14768161 | 14768400 | − | chrX | 150230485 | 150230574 | + |
| chr18 | 74219708 | 74220049 | − | chr4 | 14768669 | 14768873 | − | chrX | 153220013 | 153220192 | + |
| chr18 | 75989212 | 75989481 | − | chr4 | 15396595 | 15396890 | − | chrX | 154656059 | 154656193 | + |
| chr18_GL383570v1_alt | 83583 | 83991 | + | chr4 | 15397244 | 15397322 | − | chrX_KI270880v1_alt | 107768 | 107898 | + |
| chr19 | 82342 | 82505 | − | chr4 | 15455761 | 15456207 | − | chrX_KI270913v1_alt | 96908 | 97038 | + |
| chr19 | 1927796 | 1928030 | − | chr4 | 15742608 | 15742909 | − | chrY | 424415 | 424545 | + |
| chr19 | 5396618 | 5396918 | − | chr4 | 15748942 | 15749333 | − | chrY | 1905974 | 1906275 | − |
| chr19 | 5425945 | 5426260 | + | chr4 | 16036676 | 16036758 | + | chrY | 2597278 | 2597414 | − |
| chr19 | 5453957 | 5454216 | − | chr4 | 16931371 | 16931633 | + | chrY | 2597826 | 2597915 | − |
| chr19 | 5465531 | 5465757 | − | chr4 | 17139026 | 17139428 | − | chrY | 3450834 | 3451085 | + |
| chr19 | 5466070 | 5466219 | − | chr4 | 17346918 | 17347378 | − | chrY | 3608243 | 3608469 | + |
| chr19 | 5514506 | 5514588 | − | chr4 | 18217736 | 18218185 | − | chrY | 6592057 | 6592166 | − |
| chr19 | 5514885 | 5515206 | − | chr4 | 19722708 | 19723082 | − | chrY | 7723833 | 7724126 | − |
| chr19 | 6446407 | 6446729 | + | chr4 | 19873474 | 19873837 | − | chrY | 11475915 | 11476167 | + |
| chr19 | 6447193 | 6447297 | + | chr4 | 19904472 | 19904801 | + | chrY | 11926113 | 11926223 | + |
| chr19 | 6642994 | 6643054 | − | chr4 | 20643864 | 20644137 | − | chrY | 12116286 | 12116500 | − |
| chr19 | 6645168 | 6645384 | − | chr4 | 21127265 | 21127387 | + | chrY | 12276117 | 12276324 | − |
| chr19 | 6886493 | 6886893 | + | chr4 | 22470729 | 22470982 | − | chrY | 13936873 | 13937352 | + |
| chr19 | 6963873 | 6964188 | + | chr4 | 22599018 | 22599232 | − | chrY | 14202980 | 14203139 | + |
| chr19 | 6971566 | 6971846 | + | chr4 | 23110521 | 23110964 | + | chrY | 14392283 | 14392333 | − |
| chr19 | 6986598 | 6986741 | + | chr4 | 23140546 | 23141025 | − | chrY | 15760392 | 15760646 | − |

### Modulators or Synthetic Polynucleic Acids

### Modulators

Provided herein are modulators of a nucleic acid molecule derived from the transposable element described herein. In some instances, the modulators act on the transposable element or a fragment thereof. In some instances, the modulators act on a transcript transcribed from the transposable element (i.e. a transposable element transcript). In some instances, the modulator activates the RNA expression level of one or more transposable element transcripts, and/or activity of the transposable element. In some instances, the modulator inhibits the RNA expression level of one or more transposable element transcripts, and/or activity of the transposable element.

In some instances, the modulator modulates the expression of the transposable element by editing one or more components that are associated with the expression of the transposable element on the genomic level. Accordingly, in some instances, the modulator modulates the expression of a relevant lncRNA with which the transposable element is embedded. In some instances, the modulator modulates the activity of relevant transcription factors and/or enhancers that are associated with the transposable element. In some instances, the modulator epigenetically targets and programs the transposable element's loci.

In some instances, the modulator modulates the expression of the transposable element by editing one or more components that are associated with the expression of the transposable element on the transcription level. Accordingly, in some instances, the modulator modulates the binding of a relevant RNA-binding protein to the transposable element. In some instances, the modulator modulates RNA modification and/or editing loci in the transcript of the transposable element alone. In some instances, the modulator modulates RNA modification and/or editing loci in the transcript of the transposable element that is embedded within a lncRNA. In some instances, the modulator modulates RNA structural features within the transcript of the transposable element alone. In some instances, the modulator modulates RNA structural features within the transcript of the transposable element that is embedded within a lncRNA.

In some instances, the modulator comprises a nucleic acid-guided endonuclease complex, wherein the nucleic acid targets the transposable element. In some instances, the modulator comprises a nucleic acid-guided nucleic acid editing complex. In some instances, the modulator is a CRISPR-directed DNA editing complex. In some instances, the modulator is a CRISPR-directed RNA editing complex. In some instances, the modulator is an ASO-directed RNA editing complex.

In some instances, the modulator is a nucleic acid-guided endonuclease complex that modulates the expression of the transposable element by editing one or more components that are associated with the expression of the transposable element on the genomic level. Accordingly, in some instances, the modulator is a nucleic acid-guided endonuclease complex that modulates the expression of a relevant lncRNA with which the transposable element is embedded. In some instances, the modulator is a nucleic-acid guided endonuclease complex that modulates the activity of relevant transcription factors and/or enhancers that are associated with the transposable element. In some instances, the modulator is a nucleic acid-guided endonuclease complex that epigenetically targets and programs the transposable element's loci. In some instances, the modulator is dCas9-KRAB/CRISPRi.

In some instances, the modulator is a nucleic-acid guided endonuclease complex that modulates the expression of the transposable element by editing one or more components that are associated with the expression of the transposable element on the transcription level. Accordingly, in some instances, the modulator is a nucleic acid-guided endonuclease complex that modulates the binding of a relevant RNA-binding protein to the transposable element. In some instances, the modulator is a nucleic acid-guided endonuclease complex that modulates RNA modification and/or editing loci in the transcript of the transposable element alone. In some instances, the modulator is a nucleic acid-guided endonuclease complex that modulates RNA modification and/or editing loci in the transcript of the transposable element that is embedded within a lncRNA. In some instances, the modulator is a nucleic acid-guided endonuclease complex that modulates RNA structural features within the transcript of the transposable element alone. In some instances, the modulator is a nucleic-acid guided endonuclease complex that modulates RNA structural features within the transcript of the transposable element that is embedded within a lncRNA.

In some instances, the nucleic acid-guided endonuclease complex is a programmable nucleic acid sequence specific endonuclease. In some instances, the nucleic acid-guided endonuclease complex is a nucleic acid-guided endonuclease. In some instances, the nucleic acid-guided endonuclease complex is a CRISPR-based tool. In other instances, the nucleic acid-guided endonuclease complex is a meganuclease-based tool. In other instances, the nucleic acid-guided endonuclease complex is a zinc finger nuclease (ZFN)-based tool. In other aspects, the nucleic acid-guided endonuclease complex is a transcription activator-like effector-based nuclease (TALEN)-based tool. In other instances, the nucleic acid-guided endonuclease complex is an Argonaute system.

In some instances, the CRISPR-based tool disclosed herein is a Type I, Type II, Type III, Type IV, Type V, or Type VI CRISPR system. CRISPR/Cas systems may be multi-protein systems or single effector protein systems. Multi-protein, or Class 1, CRISPR systems include Type I, Type III, and Type IV systems. In some instances, Class 2 systems include a single effector molecule and include Type II, Type V, and Type VI. In some instances, the CRISPR-based tool disclosed herein comprises a single or multiple effector proteins. An effector protein may comprise one or multiple nuclease domains. An effector protein may target DNA or RNA, and the DNA or RNA may be single stranded or double stranded. Effector proteins may generate double strand or single strand breaks. Effector proteins may comprise mutations in a nuclease domain thereby generating a nickase protein. Effector proteins may comprise mutations in one or more nuclease domains, thereby generating a catalytically dead nuclease that is able to bind but not cleave a target sequence.

In some instances, the CRISPR-based tool disclosed comprises a single or multiple guiding RNAs (gRNAs). In some instances, the gRNA disclosed herein targets a portion of chr3: 45818666-45818847. In some instances, the gRNA disclosed herein targets a portion of chr21: 42041909-42042242. The gRNA may comprise a crRNA. The gRNA may comprise a chimeric RNA with crRNA and tracrRNA sequences. The gRNA may comprise a separate crRNA and tracrRNA. Target nucleic acid sequences may comprise a protospacer adjacent motif (PAM) or a protospacer flanking site (PFS). The PAM or PFS may be 3′ or 5′ of the target or protospacer site. Cleavage of a target sequence may generate blunt ends, 3′ overhangs, or 5′ overhangs.

The gRNA disclosed herein may comprise a spacer sequence. Spacer sequences may be complementary to target sequences or protospacer sequences. Spacer sequences may be 10, 11, 12, 13, 14, 15, 16, 17, 18, 19, 20, 21, 22, 23, 24, 25, 26, 27, 28, 29, 30, 31, 32, 33, 34, 35, or 36 nucleotides in length. In some instances, the spacer sequence may be less than 10 or more than 36 nucleotides in length.

The gRNA disclosed herein may comprise a repeat sequence. In some instances, the repeat sequence is part of a double stranded portion of the gRNA. A repeat sequence may be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some instances, the spacer sequence may be less than 10 or more than 50 nucleotides in length.

The gRNA disclosed herein may comprise one or more synthetic nucleotides, non-naturally occurring nucleotides, nucleotides with a modification, deoxyribonucleotide, or any combination thereof. Additionally and/or alternatively, a gRNA may comprise a hairpin, linker region, single stranded region, double stranded region, or any combination thereof. Additionally or alternatively, a gRNA may comprise a signaling or reporter molecule.

The gRNA disclosed herein may be encoded by genetic or episomal DNA. The gRNA disclosed herein may be provided or delivered concomitantly with a CRISPR nuclease or sequentially. The gRNA disclosed herein may be chemically synthesized, in vitro transcribed or otherwise generated using standard RNA generation techniques known in the art.

The CRISPR-based tool disclosed herein can be a Type II CRISPR system, for example a Cas9 system. The Type II nuclease can comprise a single effector protein, which, In some instances, comprises a RuvC and HNH nuclease domains. In some instances, a functional Type II nuclease may comprise two or more polypeptides, each of which comprises a nuclease domain or fragment thereof. The target nucleic acid sequences may comprise a 3′ protospacer adjacent motif (PAM). In some instances, the PAM may be 5′ of the target nucleic acid. Guide RNAs (gRNA) may comprise a single chimeric gRNA, which contains both crRNA and tracrRNA sequences. In some instances, the gRNA may comprise a set of two RNAs, for example a crRNA and a tracrRNA. The Type II nuclease may generate a double strand break, which in some cases creates two blunt ends. In some instances, the Type II CRISPR nuclease is engineered to be a nickase such that the nuclease only generates a single strand break. In such cases, two distinct nucleic acid sequences may be targeted by gRNAs such that two single strand breaks are generated by the nickase. In some instances, the two single strand breaks effectively create a double strand break. In some instances where a Type II nickase is used to generate two single strand breaks, the resulting nucleic acid free ends may either be blunt, have a 3′ overhang, or a 5′ overhang. In some instances, a Type II nuclease may be catalytically dead such that it binds to a target sequence, but does not cleave. For example, a Type II nuclease may have mutations in both the RuvC and HNH domains, thereby rendering the both nuclease domains non-functional. A Type II CRISPR system may be one of three sub-types, namely Type II-A, Type II-B, or Type II-C.

The CRISPR-based tool disclosed herein can be a Type V CRISPR system, for example a Cpf1, C2c1, or C2c3 system. The Type V nuclease may comprise a single effector protein, which comprises a single RuvC nuclease domain. In other cases, a function Type V nuclease comprises a RuvC domain split between two or more polypeptides. In such cases, the target nucleic acid sequences may comprise a 5′ PAM or 3′ PAM. Guide RNAs (gRNA) may comprise a single gRNA or single crRNA, such as may be the case with Cpf1. In some instances, a tracrRNA is not needed. In other examples, such as when C2c1 is used, a gRNA may comprise a single chimeric gRNA, which contains both crRNA and tracrRNA sequences or the gRNA may comprise a set of two RNAs, for example a crRNA and a tracrRNA. The Type V CRISPR nuclease may generate a double strand break, which generates a 5′ overhang. In some instances, the Type V CRISPR nuclease is engineered to be a nickase such that the nuclease only generates a single strand break. In such cases, two distinct nucleic acid sequences may be targeted by gRNAs such that two single strand breaks are generated by the nickase. In some instances, the two single strand breaks effectively create a double strand break. In some instances where a Type V nickase is used to generate two single strand breaks, the resulting nucleic acid free ends may either be blunt, have a 3′ overhang, or a 5′ overhang. In some instances, a Type V nuclease may be catalytically dead such that it binds to a target sequence, but does not cleave. For example, a Type V nuclease may have mutations a RuvC domain, thereby rendering the nuclease domain non-functional.

The CRISPR-based tool disclosed herein may be a Type VI CRISPR system, for example a C2c2 system. A Type VI nuclease may comprise a HEPN domain. In some instances, the Type VI nuclease comprises two or more polypeptides, each of which comprises a HEPN nuclease domain or fragment thereof. In such cases, the target nucleic acid sequences may by RNA, such as single stranded RNA. When using Type VI CRISPR system, a target nucleic acid may comprise a protospacer flanking site (PFS). The PFS may be 3′ or 5′ or the target or protospacer sequence. Guide RNAs (gRNA) may comprise a single gRNA or single crRNA. In some instances, a tracrRNA is not needed. In other examples, a gRNA may comprise a single chimeric gRNA, which contains both crRNA and tracrRNA sequences or the gRNA may comprise a set of two RNAs, for example a crRNA and a tracrRNA. In some instances, a Type VI nuclease may be catalytically dead such that it binds to a target sequence, but does not cleave. For example, a Type VI nuclease may have mutations in a HEPN domain, thereby rendering the nuclease domains non-functional.

Non-limiting examples of suitable nucleases, including nucleic acid-guided nucleases, for use in the present disclosure include C2c1, C2c2, C2c3, Cas1, Cas1B, Cas2, Cas3, Cas4, Cas5, Cas6, Cas7, Cas8, Cas9 (also known as Csn1 and Csx12), Cas10, Cpf1, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx100, Csx16, CsaX, Csx3, Csx1, Csx15, Csf1, Csf2, Csf3, Csf4, homologues thereof, orthologues thereof, or modified versions thereof.

In some instances, The CRISPR-based tool disclosed herein is an Argonaute (Ago) system. Ago protein may be derived from a prokaryote, eukaryote, or archaea. The target nucleic acid may be RNA or DNA. A DNA target may be single stranded or double stranded. In some instances, the target nucleic acid does not require a specific target flanking sequence, such as a sequence equivalent to a protospacer adjacent motif or protospacer flanking sequence. The Ago protein may create a double strand break or single strand break. In some instances, when an Ago protein forms a single strand break, two Ago proteins may be used in combination to generate a double strand break. In some instances, an Ago protein comprises one, two, or more nuclease domains. In some instances, an Ago protein comprises one, two, or more catalytic domains. One or more nuclease or catalytic domains may be mutated in the Ago protein, thereby generating a nickase protein capable of generating single strand breaks. In other aspects, mutations in one or more nuclease or catalytic domains of an Ago protein generates a catalytically dead Ago protein that may bind but not cleave a target nucleic acid.

Ago proteins may be targeted to target nucleic acid sequences by a guiding nucleic acid. In some instances, the guiding nucleic acid is a guide DNA (gDNA). The gDNA may have a 5′ phosphorylated end. The gDNA may be single stranded or double stranded. Single stranded gDNA may be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some instances, the gDNA may be less than 10 nucleotides in length. In some instances, the gDNA may be more than 50 nucleotides in length.

Argonaute-mediated cleavage may generate blunt end, 5′ overhangs, or 3′ overhangs. In some instances, one or more nucleotides are removed from the target site during or following cleavage.

In some instances, the nucleic-acid guided endonuclease complex is a repressive dCas9 with the aid of a (single) guide RNA targeting the portion of the genomic region that is transcribed to the long noncoding transcript. In some instances, the nucleic-acid guided endonuclease complex is dCas9-KRAB-MECP2 with the aid of a (single) guide RNA targeting the portion of the genomic region that is transcribed to the long noncoding transcript. In other specific aspects, the nucleic-acid guided endonuclease complex is dCas9-KRAB-DNMTI with the aid of a (single) guide RNA targeting the portion of the genomic region that is transcribed to the long noncoding transcript. In the above-mentioned aspects, the (single) guide RNA targets 5′ side of an enhancer region the genomic region that is transcribed to the long noncoding transcript. In the certain aspects, the (single) guide RNA targets 5′ side of an enhancer region the genomic region that is transcribed to the long noncoding transcript.

In some instances, the modulator comprises a nucleic acid molecule that hybridizes to a transcript of the transposable element. In some instances, the nucleic acid molecule is a small interfering RNA (siRNA), a microRNA (miRNA), an inhibitory double stranded RNA (dsRNA), a small or short hairpin RNA (shRNA), an antisense oligonucleotide (ASO), a piwi-interacting RNA (piRNA), a heterogeneous nuclear RNA (hnRNA), a small nuclear RNA (snRNA), or an enzymatically-prepared siRNA (esiRNA) or the precursors thereof. In some instances, the nucleic acid molecule is single-stranded. In some instances, the nucleic acid is double-stranded. In some instances, the nucleic acid molecule is an ASO. In some instances, the ASO is a GapmeR (or Gapmer, both are used interchangeably) or a MixmeR (or mixmer, both are used interchangeably).

In some instances, the ASO is about 6-50 nucleotides long. In some instances, the ASO is about 6-45, 6-40, 6-35, 6-30, 6-20, 6-18, 7-45, 7-40, 7-35, 7-30, 7-20, 7-18, 8-45, 8-40, 8-35, 8-30, 8-20, 8-18, 9-45, 9-40, 9-35, 9-30, 9-20, 9-18, 10-45, 10-40, 10-35, 10-20, 10-18, 11-30, 11-45, 11-40, 11-35, 11-30, 11-20, 11-18, 12-45, 12-40, 12-35, 12-30, 12-20, or 12-18 nucleotides long. In some instances, the ASO is about 12-30 nucleotides long. In some instances, the ASO is at least 6, 7, 8, 9, or 10 nucleotides long. In some instances, the ASO is at most 18, 20, 30, 35, 40, 45, 50, 55, or 60 nucleotides long.

In some instances, the ASO binds to an RNA transcript transcribed from a nucleic acid sequence having a sequence homology of at least 55%, 60%, 65%, 70%, 75%, 80%, 85%, 90% from at least a portion of SEQ ID NO: 4, 7-11, and 14-121. In some instances, the ASO comprises a nucleic acid sequence that is complementary to a portion of DNA sequence having a sequence homology of at least 55%, 60%, 65%, 70%, 75%, 80%, 85%, 90% from at least a portion of SEQ ID NO: 4, 7-11, and 14-121. In some instances, the ASO binds to an RNA transcript transcribed from a nucleic acid sequence having a sequence homology of at least 55%, 60%, 65%, 70%, 75%, 80%, 85%, 90% from at least a portion of SEQ ID NO: 4, 10, 14, 15, 18, 27-32, 34-38. In some instances, the ASO comprises a nucleic acid sequence that is complementary to a portion of DNA sequence having a sequence homology of at least 55%, 60%, 65%, 70%, 75%, 80%, 85%, 90% from at least a portion of SEQ ID NO: 4, 10, 14, 15, 18, 27-32, 34-38. In some instances, the ASO comprises at least 9 consecutive nucleotides with 1 mismatch from SEQ ID NOs: 1-3. In some instances, the ASO comprises at least 9 consecutive nucleotides with 2 mismatches from SEQ ID NOs: 1-3. In some instances, the ASO comprises at least 9 consecutive nucleotides with 3 mismatches from SEQ ID NOs: 1-3. In some instances, the ASO comprises at least 10 consecutive nucleotides with 1 mismatch from SEQ ID NOs: 1-3. In some instances, the ASO comprises at least 10 consecutive nucleotides with 2 mismatches from SEQ ID NOs: 1-3. In some instances, the ASO comprises at least 10 consecutive nucleotides with 3 mismatches from SEQ ID NOs: 1-3. In some instances, the ASO comprises at least 11 consecutive nucleotides with 1 mismatch from SEQ ID NOs: 1-3. In some instances, the ASO comprises at least 11 consecutive nucleotides with 2 mismatches from SEQ ID NOs: 1-3. In some instances, the ASO comprises at least 11 consecutive nucleotides with 3 mismatches from SEQ ID NOs: 1-3. In some instances, the ASO comprises at least 12 consecutive nucleotides with 1 mismatch from SEQ ID NOs: 1-3. In some instances, the ASO comprises at least 12 consecutive nucleotides with 2 mismatches from SEQ ID NOs: 1-3. In some instances, the ASO comprises at least 12 consecutive nucleotides with 3 mismatches from SEQ ID NOs: 1-3. In some instances, the ASO comprises at least 13 consecutive nucleotides with 1 mismatch from SEQ ID NOs: 1-3. In some instances, the ASO comprises at least 13 consecutive nucleotides with 2 mismatches from SEQ ID NOs: 1-3. In some instances, the ASO comprises at least 13 consecutive nucleotides with 3 mismatches from SEQ ID NOs: 1-3.

In some instances, the modulator described herein comprises one or more sugar-modified nucleotide. In some specific instances, the sugar-modified nucleotide is a 2′-fluoro modified nucleotide. In some specific instances, the sugar-modified nucleotide is a 2′-alkoxy modified nucleotide (e.g., 2′-methoxy modified nucleotide). In some specific instances, the sugar-modified nucleotide is a 2′-amino modified nucleotide. In some specific instances, the sugar-modified nucleotide is a 2′-azido modified nucleotide.

In some instances, the modulator described herein comprises one or more backbone-modified nucleotide. In some specific instances, the modified backbone is a methylphosphonate internucleotide linkage. In some specific instances, the modified backbone is phosphorothioate internucleotide linkage. In some specific instances, the modified backbone is a guanidinopropyl phosphoramidate internucleotide linkage. In some specific instances, the modified backbone is a mesyl-phosphoramidate (MsPA) linkages.

In some specific instances, the modified backbone is phosphorothioate internucleotide linkage, and the phosphorothioate is a stereochemically enriched phosphorothioate internucleotide linkage. In certain instances, the strand contains at least one stereochemically enriched phosphorothioate internucleotide linkage. In some instances, the strand comprises at least 1, 2, 3 stereochemically enriched phosphorothioates internucleotide linkage. In some instances, the strand comprises only 1, 2, 3, or 4 stereochemically enriched phosphorothioates internucleotide linkage.

In some instances, the modulator described herein comprises one or more purine modification. In some specific instances, the purine modification described herein is 2,6-diaminopurine. In some specific instances, the purine modification described herein is 3-deaza-adenine. In some specific instances, the purine modification described herein is 7-deaza-guanine. In some specific instances, the purine modification described herein is 8-azido-adenine.

In some instances, the modulator described herein comprises one or more pyrimidine modification. In some specific instances, the pyrimidine modification described herein is 2-thio-thymidine. In some specific instances, the pyrimidine modification described herein is 5-carboxamide-uracil. In some specific instances, the pyrimidine modification described herein is 5-methyl-cytosine. In some specific instances, the pyrimidine modification described herein is 5-ethynyl uracil.

In some instances, the modulator described herein comprises an abasic substitution. In those cases where a hybridized polynucleotide construct is contemplated for use as siRNA, a reduction of miRNA-like off-target effects is desirable. The inclusion of one or more (e.g., one or two) abasic substitutions in the hybridized polynucleotide constructs may reduce or even eliminate miRNA-like off-target effects, as the abasic substitutions lack nucleobases that are capable of engaging in base-pairing interactions and alleviate steric hindrance. Thus, the modulator disclosed herein may include one or more (e.g., one or two) abasic substitutions. In specific instances, abasic substitution is at the 5th nucleotide from the 5′ end of the antisense strand described herein. The modulator described herein may contain a strand including a seed region including a hypoxanthine nucleobase-containing nucleoside (e.g., inosine).

In some instances, the modulator described herein comprises one or more type of modifications as described above. Accordingly, in some instances, about 10% of the nucleotides from the modulator described herein are modified with one or more type of modifications as described above. In other instances, about 20% of the nucleotides from the modulator described herein are modified with one or more type of modifications as described above. In other instances, about 30% of the nucleotides from the modulator described herein are modified with one or more type of modifications as described above. In other instances, about 40% of the nucleotides from the modulator described herein are modified with one or more type of modifications as described above. In other instances, about 50% of the nucleotides from the modulator described herein are modified with one or more type of modifications as described above. In other instances, about 60% of the nucleotides from the modulator described herein are modified with one or more type of modifications as described above. In other instances, about 70% of the nucleotides from the modulator described herein are modified with one or more type of modifications as described above. In other instances, about 80% of the nucleotides from the modulator described herein are modified with one or more type of modifications as described above. In other instances, about 90% of the nucleotides from the modulator described herein are modified with one or more type of modifications as described above. In other instances, 100% of the nucleotides from the modulator described herein are modified with one or more type of modifications as described above.

In some instances, the one or more types of modifications described herein occurs at different positions within the modulator described herein. In specific instances, the one or more types of modifications described herein occurs in the seed region within the modulator described herein. In specific instances, the one or more types of modifications described herein occurs at 3′ terminal of the modulator described herein. In specific instances, the one or more types of modifications described herein occurs at 5′ terminal of the modulator described herein. In specific instances, the one or more types of modifications described herein occurs dispersedly within the modulator described herein. In specific instances, the one or more types of modifications described herein occurs in clusters within the modulator described herein.

In some instances, the ASO is a Gapmer comprising a central region of consecutive DNA nucleotides flanked by a 5′-wing region and 3′-wing region, wherein at least one of 5′-wing region and 3′-wing region comprises a nucleic acid analogue, wherein the nucleic acid analogue comprises one or more ribose modifications, one or more backbone modifications, one or more nucleobase modifications, or a combination thereof.

In some instances, the one or more ribose modifications disclosed herein include locked nucleic acid (LNA), tricyclo-DNA, 2′-fluoro, 2′-O-methyl, 2′-methoxyethyl(2′-MOE), 2′-cyclic ethyl (CET), unlocked nucleic acid (UNA), conformationally restricted nucleoside (CRN), or any combination thereof. In some instances, the one or more backbone modifications comprise phosphorothioate, methylphosphonate, guanidinopropyl phosphoramidate, or any combination thereof. In some instances, the one or more nucleobases comprise purine modifications (e.g., 2,6-diaminopurin, 3-deaza-adenine, 7-deaza-guanine, 8-zaido-adenine, or any combination thereof). In some instances, the one or more nucleobases comprise pyrimidine modifications (e.g., 2-thio-thymidine, 5-carboxamide-uracil, 5-methyl-cytosine, 5-ethynyl-uracil, or any combination thereof).

In some instances, the nucleic acid analogue comprises an LNA. In some instances, the LNA comprises a beta-D-oxy LNA, an alpha-L-oxy-LNA, a beta-D-amino-LNA, an alpha-L-amino-LNA, a beta-D-thio-LNA, an alpha-L-thio-LNA, a 5′-methyl-LNA, a beta-D-ENA, or an alpha-L-ENA. In some instances, the LNA comprises a beta-D-oxy LNA. In some instances, the 5′-wing region comprises at least one LNA. In some instances, the 5′-wing region comprises at least two LNAs. In some instances, the 5′-wing region comprises at least three LNAs. In some instances, the 5′-wing region comprises at least four LNAs. In some instances, the 5′-wing region comprises two consecutive LNAs. In some instances, the 5′-wing region comprises three consecutive LNAs. In some instances, the 5′-wing region comprises four consecutive LNAs. In some instances, the 3′-wing region comprises at least one LNA. In some instances, the 3′-wing region comprises at least two LNA. In some instances, the 3′-wing region comprises at least three LNA. In some instances, the 3′-wing region comprises at least four LNA. In some instances, the 3′-wing region comprises two consecutive LNAs. In some instances, the 3′-wing region comprises three consecutive LNAs. In some instances, the 3′-wing region comprises four consecutive LNAs. In some instances, the Gapmer has a 3-10-3, 4-10-2, 2-10-4, 3-9-4, 4-9-3, 3-11-2, 2-11-3, 1-11-4, or 4-11-1 configuration, wherein the first number refers to the number of LNAs as a 5′-wing region, the second number refers to the number of consecutive DNA nucleotides in the central region, and the third number refers to the number of LNAs as a 3′-wing region. In some instances, one or more phosphodiester backbone is a phosphorothioate backbone. In some instances, each phosphodiester backbone is a phosphorothioate backbone.

In some instances, the nucleic acid analogue comprises 2′-MOE modified nucleotide. In some instances, the 5′-wing region comprises at least one 2′-MOE modified nucleotide. In some instances, the 5′-wing region comprises at least two 2′-MOE modified nucleotides. In some instances, the 5′-wing region comprises at least three 2′-MOE modified nucleotides. In some instances, the 5′-wing region comprises at least four 2′-MOE modified nucleotides. In some instances, the 5′-wing region comprises two consecutive 2′-MOE modified nucleotides. In some instances, the 5′-wing region comprises three consecutive 2′-MOE modified nucleotides. In some instances, the 5′-wing region comprises four consecutive 2′-MOE modified nucleotides. In some instances, the 3′-wing region comprises at least one 2′-MOE modified nucleotide. In some instances, the 3′-wing region comprises at least two 2′-MOE modified nucleotide. In some instances, the 3′-wing region comprises at least three 2′-MOE modified nucleotide. In some instances, the 3′-wing region comprises at least four 2′-MOE modified nucleotide. In some instances, the 3′-wing region comprises two consecutive 2′-MOE modified nucleotides. In some instances, the 3′-wing region comprises three consecutive 2′-MOE modified nucleotides. In some instances, the 3′-wing region comprises four consecutive 2′-MOE modified nucleotides. In some instances, the Gapmer has a 3-10-3, 4-10-2, 2-10-4, 3-9-4, 4-9-3, 3-11-2, 2-11-3, 1-11-4, 4-11-1, or 5-10-5 configuration, wherein the first number refers to the number of 2′-MOE modified nucleotides as a 5′-wing region, the second number refers to the number of consecutive DNA nucleotides in the central region, and the third number refers to the number of 2′-MOE modified nucleotide as a 3′-wing region. In some instances, one or more phosphodiester backbone is a phosphorothioate backbone. In some instances, each phosphodiester backbone is a phosphorothioate backbone.

In some instances, the nucleic acid analogue comprises both 2′-MOE modified nucleotide and LNA. In some instances, the 5′-wing region comprises at least one 2′-MOE modified nucleotide or LNA. In some instances, the 5′-wing region comprises at least two nucleic acid analogues which is a mixture of 2′-MOE modified nucleotide(s) and LNA(s). In some instances, the 5′-wing region comprises at least three nucleic acid analogues which is a mixture of 2′-MOE modified nucleotide(s) and LNA(s). In some instances, the 5′-wing region comprises at least four nucleic acid analogues which is a mixture of 2′-MOE modified nucleotide(s) and LNA(s). In some instances, the 5′-wing region comprises two consecutive nucleic acid analogues which is a mixture of 2′-MOE modified nucleotide(s) and LNA(s). In some instances, the 5′-wing region comprises three consecutive nucleic acid analogues which is a mixture of 2′-MOE modified nucleotide(s) and LNA(s). In some instances, the 5′-wing region comprises four consecutive nucleic acid analogues which is a mixture of 2′-MOE modified nucleotide(s) and LNA(s). In some instances, the 3′-wing region comprises at least one LNA. In some instances, the 3′-wing region comprises at least two LNA. In some instances, the 3′-wing region comprises at least three LNA. In some instances, the 3′-wing region comprises at least four LNA. In some instances, the 3′-wing region comprises two consecutive nucleic acid analogues which is a mixture of 2′-MOE modified nucleotide(s) and LNA(s). In some instances, the 3′-wing region comprises three consecutive nucleic acid analogues which is a mixture of 2′-MOE modified nucleotide(s) and LNA(s). In some instances, the 3′-wing region comprises four consecutive nucleic acid analogues which is a mixture of 2′-MOE modified nucleotide(s) and LNA(s). In some instances, the Gapmer has a 3-10-3, 4-10-2, 2-10-4, 3-9-4, 4-9-3, 3-11-2, 2-11-3, 1-11-4, 4-11-1, or 5-10-5 configuration, wherein the first number refers to the number of a mixture of 2′-MOE modified nucleotide(s) and LNA(s) as a 5′-wing region, the second number refers to the number of consecutive DNA nucleotides in the central region, and the third number refers to the number of a mixture of 2′-MOE modified nucleotide(s) and LNA(s) as a 3′-wing region. In some instances, one or more phosphodiester backbone is a phosphorothioate backbone. In some instances, each phosphodiester backbone is a phosphorothioate backbone.

In some instances, the modulator is a Gapmer, and one or more assays are utilized to assess the efficiency of the Gapmer. Accordingly, in some instances, the efficiency of the modulator is assessed by the expression (e.g., transcript expression) of the transposable element. In some instances, the efficiency of the modulator is assessed by the expression of the transposable element that is embedded in a relevant lncRNA. In some instances, the efficiency of the modulator is assessed by the features of the transposable element. In some instances, the efficiency of the modulator is assessed by the features of the transposable element that is embedded in a relevant lncRNA. In some instances, the efficiency of the modulator is assessed by the binding of a transcription factor and/or an enhancer to the transposable element. In some instances, the efficiency of the modulator is assessed by the binding of a transcription factor and/or an enhancer to the transposable element that is embedded in a relevant lncRNA. In some instances, the efficiency of the modulator is assessed by the binding of an RNA-binding protein to the transposable element. In some instances, the efficiency of the modulator is assessed by the binding of an RNA-binding protein to the transposable element that is embedded in a relevant lncRNA. In some instances, the efficiency of the modulator is assessed by the modification and edit features of the transposable element. In some instances, the efficiency of the modulator is assessed by the modification and edit features of the transposable element that is embedded in a relevant lncRNA.

In some instances, the modulator is a mixmer, and one or more assays are utilized to assess the efficiency of the mixmer Accordingly, in some instances, the efficiency of the modulator is assessed by the expression of the transposable element. In some instances, the efficiency of the modulator is assessed by the expression of the transposable element that is embedded in a relevant lncRNA. In some instances, the efficiency of the modulator is assessed by the features of the transposable element. In some instances, the efficiency of the modulator is assessed by the features of the transposable element that is embedded in a relevant lncRNA. In some instances, the efficiency of the modulator is assessed by the binding of a transcription factor and/or an enhancer to the transposable element. In some instances, the efficiency of the modulator is assessed by the binding of a transcription factor and/or an enhancer to the transposable element that is embedded in a relevant lncRNA. In some instances, the efficiency of the modulator is assessed by the binding of an RNA-binding protein to the transposable element. In some instances, the efficiency of the modulator is assessed by the binding of an RNA-binding protein to the transposable element that is embedded in a relevant lncRNA. In some instances, the efficiency of the modulator is assessed by the modification and edit features of the transposable element. In some instances, the efficiency of the modulator is assessed by the modification and edit features of the transposable element that is embedded in a relevant lncRNA.

In some instances, the modulator is an siRNA which targets a cytoplasmic target, and one or more assays are utilized to assess the efficiency of the modulator. Accordingly, in some instances, the efficiency of the modulator is assessed by the expression of the transposable element. In some instances, the efficiency of the modulator is assessed by the expression of the transposable element that is embedded in a relevant lncRNA. In some instances, the efficiency of the modulator is assessed by the features of the transposable element. In some instances, the efficiency of the modulator is assessed by the features of the transposable element that is embedded in a relevant lncRNA. In some instances, the efficiency of the modulator is assessed by the binding of an RNA-binding protein to the transposable element. In some instances, the efficiency of the modulator is assessed by the binding of an RNA-binding protein to the transposable element that is embedded in a relevant lncRNA. In some instances, the efficiency of the modulator is assessed by the modification and/or edit features of the transposable element. In some instances, the efficiency of the modulator is assessed by the modification and/or edit features of the transposable element that is embedded in a relevant lncRNA. In some instances, the efficiency of the modulator is assessed by structural features of the transposable element. In some instances, the efficiency of the modulator is assessed by structural features of the transposable element that is embedded in a relevant lncRNA.

In some instances, the modulator described herein targets one of the two flanking LTR16C regions within chr3: 45,818,666-45,818,847 (hg38) or a transcript molecule transcribed therefrom. In some instances, the modulator described herein targets chr3: 45,818,736-45,818,762 (hg38) or a transcript molecule transcribed therefrom. In some instances, the modulator described herein targets chr3: 45,818,689-45,818,712 (hg38) or a transcript molecule transcribed therefrom. In some instances, the modulator described herein targets genomic positions of LTR16C disclosed in Table 2 or a transcript molecule transcribed therefrom. In some instances, the modulator described herein targets ERV region within chr3: 45,818,666-45,818,847 or a transcript molecule transcribed therefrom.

Further provided herein is a modulator comprising an antisense oligonucleotide (ASO), wherein the ASO comprises at least 9 consecutive nucleotides with no more than 3 mismatches from 5′-CGGAGGCATGAGGTAG-3′ (SEQ ID NO: 1). Accordingly, in some instances, the ASO comprises at least 9 consecutive nucleotides with 1 mismatch from SEQ ID NO: 1. In some instances, the ASO comprises at least 9 consecutive nucleotides with 2 mismatches from SEQ ID NO: 1. In some instances, the ASO comprises at least 9 consecutive nucleotides with 3 mismatches from SEQ ID NO: 1. In some instances, the ASO comprises at least 10 consecutive nucleotides with 1 mismatch from SEQ ID NO: 1. In some instances, the ASO comprises at least 10 consecutive nucleotides with 2 mismatches from SEQ ID NO: 1. In some instances, the ASO comprises at least 10 consecutive nucleotides with 3 mismatches from SEQ ID NO: 1. In some instances, the ASO comprises at least 11 consecutive nucleotides with 1 mismatch from SEQ ID NO: 1. In some instances, the ASO comprises at least 11 consecutive nucleotides with 2 mismatches from SEQ ID NO: 1. In some instances, the ASO comprises at least 11 consecutive nucleotides with 3 mismatches from SEQ ID NO: 1. In some instances, the ASO comprises at least 12 consecutive nucleotides with 1 mismatch from SEQ ID NO: 1. In some instances, the ASO comprises at least 12 consecutive nucleotides with 2 mismatches from SEQ ID NO: 1. In some instances, the ASO comprises at least 12 consecutive nucleotides with 3 mismatches from SEQ ID NO: 1. In some instances, the ASO comprises at least 13 consecutive nucleotides with 1 mismatch from SEQ ID NO: 1. In some instances, the ASO comprises at least 13 consecutive nucleotides with 2 mismatches from SEQ ID NO: 1. In some instances, the ASO comprises at least 13 consecutive nucleotides with 3 mismatches from SEQ ID NO: 1. In some instances, the ASO comprises a nucleic acid sequence of 80%, at least 85%, at least 90%, at least 95% identical to SEQ ID NO: 1.

Further provided herein is a modulator comprising an antisense oligonucleotide (ASO), wherein the ASO comprises at least 9 consecutive nucleotides with no more than 3 mismatches from 5′-TGAGCAGGTTAGCACT-3′ (SEQ ID NO: 2). Accordingly, in some instances, the ASO comprises at least 9 consecutive nucleotides with 1 mismatch from SEQ ID NO: 2. In some instances, the ASO comprises at least 9 consecutive nucleotides with 2 mismatches from SEQ ID NO: 2. In some instances, the ASO comprises at least 9 consecutive nucleotides with 3 mismatches from SEQ ID NO: 2. In some instances, the ASO comprises at least 10 consecutive nucleotides with 1 mismatch from SEQ ID NO: 2. In some instances, the ASO comprises at least 10 consecutive nucleotides with 2 mismatches from SEQ ID NO: 2. In some instances, the ASO comprises at least 10 consecutive nucleotides with 3 mismatches from SEQ ID NO: 2. In some instances, the ASO comprises at least 11 consecutive nucleotides with 1 mismatch from SEQ ID NO: 2. In some instances, the ASO comprises at least 11 consecutive nucleotides with 2 mismatches from SEQ ID NO: 2. In some instances, the ASO comprises at least 11 consecutive nucleotides with 3 mismatches from SEQ ID NO: 2. In some instances, the ASO comprises at least 12 consecutive nucleotides with 1 mismatch from SEQ ID NO: 2. In some instances, the ASO comprises at least 12 consecutive nucleotides with 2 mismatches from SEQ ID NO: 2. In some instances, the ASO comprises at least 12 consecutive nucleotides with 3 mismatches from SEQ ID NO: 2. In some instances, the ASO comprises at least 13 consecutive nucleotides with 1 mismatch from SEQ ID NO: 2. In some instances, the ASO comprises at least 13 consecutive nucleotides with 2 mismatches from SEQ ID NO: 2. In some instances, the ASO comprises at least 13 consecutive nucleotides with 3 mismatches from SEQ ID NO: 2. In some instances, the ASO comprises a nucleic acid sequence of 80%, at least 85%, at least 90%, at least 95% identical to SEQ ID NO: 2.

Further provided herein is a modulator comprising an antisense oligonucleotide (ASO), wherein the ASO comprises at least 9 consecutive nucleotides with no more than 3 mismatches from 5′-CTACCTCATGCCTCCG-3′ (SEQ ID NO: 3). Accordingly, in some instances, the ASO comprises at least 9 consecutive nucleotides with 1 mismatch from SEQ ID NO: 3. In some instances, the ASO comprises at least 9 consecutive nucleotides with 2 mismatches from SEQ ID NO: 3. In some instances, the ASO comprises at least 9 consecutive nucleotides with 3 mismatches from SEQ ID NO: 3. In some instances, the ASO comprises at least 10 consecutive nucleotides with 1 mismatch from SEQ ID NO: 3. In some instances, the ASO comprises at least 10 consecutive nucleotides with 2 mismatches from SEQ ID NO: 3. In some instances, the ASO comprises at least 10 consecutive nucleotides with 3 mismatches from SEQ ID NO: 3. In some instances, the ASO comprises at least 11 consecutive nucleotides with 1 mismatch from SEQ ID NO: 3. In some instances, the ASO comprises at least 11 consecutive nucleotides with 2 mismatches from SEQ ID NO: 3. In some instances, the ASO comprises at least 11 consecutive nucleotides with 3 mismatches from SEQ ID NO: 3. In some instances, the ASO comprises at least 12 consecutive nucleotides with 1 mismatch from SEQ ID NO: 3. In some instances, the ASO comprises at least 12 consecutive nucleotides with 2 mismatches from SEQ ID NO: 3. In some instances, the ASO comprises at least 12 consecutive nucleotides with 3 mismatches from SEQ ID NO: 3. In some instances, the ASO comprises at least 13 consecutive nucleotides with 1 mismatch from SEQ ID NO: 3. In some instances, the ASO comprises at least 13 consecutive nucleotides with 2 mismatches from SEQ ID NO: 3. In some instances, the ASO comprises at least 13 consecutive nucleotides with 3 mismatches from SEQ ID NO: 3. In some instances, the ASO comprises a nucleic acid sequence of 80%, at least 85%, at least 90%, at least 95% identical to SEQ ID NO: 3.

### Synthetic Polynucleic Acid

In order to manipulate the endogenous processes or features of the transposable element described herein to enhance its interaction with its molecular binding partners (e.g. DNA, RNA, or protein partners), further provided herein is a synthetic polynucleic acid. In some instances, the synthetic polynucleic acid comprises a nucleic acid sequence of the transposable element described herein (e.g., in the sub-section of “Regulatory Transposable Element” or “Regulatory Transposable Element associated with Fibrosis”). In some instances, the synthetic polynucleic acid comprises a nucleic acid sequence of a functional variant of the transposable element described herein. In some instances, the synthetic polynucleic acid comprises a nucleic acid sequence of a functional fragment of the transposable element described herein. In some instances, the synthetic polynucleic acid comprises a nucleic acid sequence of a cDNA complementary to at least a portion of a transcript transcribed from the transposable element described herein.

In some instances, transfection for introducing the synthetic polynucleic acid into a donor cell (e.g., a myofibroblast in subject with lung fibrosis) comprises the use of DNA and RNA vectors. In some cases, the transfection is using viral vectors, and especially retroviral vectors. Other viral vectors, in some cases, are derived from lentivirus, poxviruses, herpes simplex virus I, adenoviruses and adeno-associated viruses, and the like. Exemplary viral vectors include retroviral vectors, adenoviral vectors, adeno-associated viral vectors (AAVs), pox vectors, parvoviral vectors, baculovirus vectors, measles viral vectors, or herpes simplex virus vectors (HSVs). In some instances, the retroviral vectors include gamma-retroviral vectors such as vectors derived from the Moloney Murine Keukemia Virus (MoMLV, MMLV, MuLV, or MLV) or the Murine Steam donor cell Virus (MSCV) genome. In some instances, the retroviral vectors also include lentiviral vectors such as those derived from the human immunodeficiency virus (HIV) genome. In some instances, AAV vectors include AAV1, AAV2, AAV4, AAV5, AAV6, AAV7, AAV8, or AAV9 serotype. In some instances, viral vector is a chimeric viral vector, comprising viral portions from two or more viruses. In additional instances, the viral vector is a recombinant viral vector.

In some instances, transfection for introducing the synthetic polynucleic acid into a donor cell (e.g., a myofibroblast in subject with lung fibrosis) comprises colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). Other methods of state-of-the-art targeted delivery of nucleic acids are available, such as delivery of the synthetic polynucleic acid with targeted nanoparticles with a corresponding antibody or other suitable sub-micron sized delivery system. In some instances, an exemplary delivery vehicle is a liposome. “Liposome” is a generic term encompassing a variety of single and multilamellar lipid vehicles formed by the generation of enclosed lipid bilayers or aggregates.

In some instances, transfection for introducing the synthetic polynucleic acid into a donor cell (e.g., a myofibroblast in vitro) comprises calcium phosphate precipitation, particle bombardment, microinjection, gene gun, electroporation, micro-needle array, nano-needle array, sonication, or chemical permeation.

In some instances, to assess the proper function of the synthetic nucleic acid, the synthetic nucleic acid's binding to an RNA-binding protein is examined. In some instances, to assess the proper function of the synthetic nucleic acid, synthetic nucleic acid's modification and edit features are examined. In some instances, to assess the proper function of the synthetic nucleic acid, synthetic nucleic acid's binding to a transcription factor and enhancer is examined.

In some instances, the synthetic polynucleic acid serves as a trans-acting functional synthetic RNA. Accordingly, in some instances, to assess the proper function of the synthetic nucleic acid, the delivery efficiency of the synthetic polynucleic acid is examined. In some instances, to assess the proper function of the synthetic nucleic acids, the relevant lncRNA that is associated with the transposable element is examined. In some instances, to assess the proper function of the synthetic nucleic acids, one or more desired trans-acting features derived from the relevant transposable element (e.g., RNA-binding protein features, RNA modification features, and/or RNA structure features etc.) are examined. In some instances, to assess the proper function of the synthetic nucleic acids, the features of a relevant trans-acting RNA-binding protein that is associated with the transposable element are examined. In some instances, to assess the proper function of the synthetic nucleic acids, the features of a relevant trans-acting RNA-binding protein that is associated with the transposable element that is embedded with a relevant lncRNA are examined.

### Pharmaceutical Compositions

Further provided herein are pharmaceutical composition comprising the modulator described herein or the synthetic polynucleic acid described herein, and a pharmaceutically acceptable salt, excipient, or derivative thereof.

The suitable pharmaceutically acceptable salts or derivative thereof include but are not limited to (i) salts formed with cations such as sodium, potassium, ammonium, magnesium, calcium, polyamines such as spermine and spermidine, etc.; (ii) acid addition salts formed with inorganic acids, for example hydrochloric acid, hydrobromic acid, sulfuric acid, phosphoric acid, and the like; and (iii) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like.

A pharmaceutical composition described herein can be prepared to include the modulator disclosed herein, into a form suitable for administration to a subject using carriers, excipients, and vehicles. In some instances, excipients include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol, and polyhydric alcohols. Intravenous vehicles include fluid and nutrient replenishers. Preservatives include antimicrobial, anti-oxidants, chelating agents, and inert gases. Other pharmaceutically acceptable vehicles include aqueous solutions, non-toxic excipients, including salts, preservatives, buffers and the like, as described, for instance, in Remington: The Science and Practice of Pharmacy, 21^{st} Ed., Gennaro, Ed., Lippencott Williams & Wilkins (2005), and The United States Pharmacopeia: The National Formulary (USP 36 NF31), published in 2013. The pH and exact concentration of the various components of the pharmaceutical composition are adjusted according to routine skills in the art. See Goodman and Gilman's, The Pharmacological Basis for Therapeutics.

The pharmaceutical compositions described herein may be administered locally or systemically. The therapeutically effective amounts will vary according to factors, such as the degree of infection in a subject, the age, sex, health conditions, and weight of the individual. Dosage regimes can be adjusted to provide the optimum therapeutic response. For example, several divided doses can be administered daily or the dose can be proportionally reduced as indicated by the exigencies of the therapeutic situation.

The pharmaceutical composition can be administered in a convenient manner, such as by injection (e.g., subcutaneous, intravenous, intraorbital, and the like), oral administration, ophthalmic application, inhalation, topical application, or rectal administration. Depending on the route of administration, the pharmaceutical composition can be coated with a material to protect the pharmaceutical composition from the action of enzymes, acids, and other natural conditions that may inactivate the pharmaceutical composition. The pharmaceutical composition can also be administered parenterally or intraperitoneally. Dispersions can also be prepared in glycerol, liquid polyethylene glycols, and mixtures thereof, and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersions. The composition can be sterile and fluid to the extent that easy syringability exists. The composition can be stable under the conditions of manufacture and storage and preserved against the contaminating action of microorganisms, such as bacteria and fungi. The vehicle can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of certain particle size, in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, isotonic agents, for example, sugars, polyalcohols, such as mannitol, sorbitol, or sodium chloride are used in the composition. Prolonged absorption of the injectable compositions can be brought about by including in the composition an agent that delays absorption, for example, aluminum monostearate and gelatin.

Sterile injectable solutions can be prepared by incorporating the pharmaceutical composition in an appropriate solvent with one or a combination of ingredients enumerated above followed by filtered sterilization. Generally, dispersions are prepared by incorporating the pharmaceutical composition into a sterile vehicle that contains a basic dispersion medium and the other ingredients from those enumerated above.

It is advantageous to formulate parenteral compositions in dosage unit form for ease of administration and uniformity of dosage. Dosage unit form as used herein, refers to physically discrete units suited as unitary dosages for the subject to be treated; each unit containing a predetermined quantity of pharmaceutical composition is calculated to produce the desired therapeutic effect in association with the pharmaceutical vehicle. The specification for the dosage unit forms is related to the characteristics of the pharmaceutical composition and the particular therapeutic effect to be achieve. The principal pharmaceutical composition is compounded for convenient and effective administration in effective amounts with a suitable pharmaceutically acceptable vehicle in an acceptable dosage unit. In the case of compositions containing supplementary active ingredients, the dosages are determined by reference to the usual dose and manner of administration of the ingredients.

The pharmaceutical composition can be orally administered, for example, in a carrier, e.g., in an enteric-coated unit dosage form. The pharmaceutical composition and other ingredients can also be enclosed in a hard or soft-shell gelatin capsule or compressed into tablets. For oral therapeutic administration, the pharmaceutical composition can be incorporated with excipients and used in the form of ingestible tablets, troches, capsules, pills, wafers, and the like. Such compositions and preparations may contain at least 1% by weight of active compound. The percentage of the compositions and preparations can, of course, be varied and can conveniently be between about 5% to about 80% of the weight of the unit. The tablets, troches, pills, capsules, and the like can also contain the following: a binder, such as gum tragacanth, acacia, corn starch, or gelatin; excipients such as dicalcium phosphate; a disintegrating agent, such as corn starch, potato starch, alginic acid, and the like; a lubricant, such as magnesium stearate; and a sweetening agent, such as sucrose, lactose or saccharin, or a flavoring agent such as peppermint, oil of wintergreen, or cherry flavoring. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar, or both. A syrup or elixir can contain the agent, sucrose as a sweetening agent, methyl and propylparabens as preservatives, a dye, and flavoring, such as cherry or orange flavor. Any material used in preparing any dosage unit form can be of pharmaceutically acceptable purity and substantially non-toxic in the amounts employed. In addition, the pharmaceutical composition can be incorporated into sustained-release preparations and formulations.

The pharmaceutical composition described herein may comprise one or more permeation enhancer that facilitates bioavailability of the modulator described herein. WO 2000/67798, Muranishi, 1990, Crit. Rev. Ther. Drug Carrier Systems, 7, 1, Lee et al., 1991, Crit. Rev. Ther. Drug Carrier Systems, 8, 91 are herein incorporated by reference in its entirety. In some aspects, the permeation enhancer is intestinal. In some aspects, the permeation enhancer is transdermal. In some aspects, the permeation enhancer is to facilitate crossing the brain-blood barrier. In some aspects, the permeation enhancer improves the permeability in the oral, nasal, buccal, pulmonary, vaginal, or corneal delivery model. In some aspects, the permeation enhancer is a fatty acid or a derivative thereof. In some aspects, the permeation enhancer is a surfactant or a derivative thereof. In some aspects, the permeation enhancer is a bile salt or a derivative thereof. In some aspects, the permeation enhancer is a chelating agent or a derivative thereof. In some aspects, the permeation enhancer is a non-chelating non-surfactant or a derivative thereof. In some aspects, the permeation enhancer is an ester or a derivative thereof. In some aspects, the permeation enhancer is an ether or a derivative thereof. In some specific aspects, the permeation enhancer is arachidonic acid, undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a monoglyceride, a diglyceride or a pharmaceutically acceptable salt thereof. In one aspect, the permeation enhancer is sodium caprate (C10). In some instances, the permeation enhancer is chenodeoxycholic acid (CDCA), ursodeoxychenodeoxycholic acid (UDCA), cholic acid, dehydrocholic acid, deoxycholic acid, glucholic acid, glycholic acid, glycodeoxycholic acid, taurocholic acid taurodeoxycholic acid, sodium tauro-24,25-dihydro-fusidate or sodium glycodihydrofusidate. In some instances, the permeation enhancer is polyoxyethylene-9-lauryl ether, or polyoxyethylene-20-cetyl ether.

### Kits

Further provided herein are kits comprising the modulator described herein or the synthetic polynucleic acid described herein, or the pharmaceutical composition described herein.

In some aspects, the kit comprises suitable instructions in order to perform the methods of the kit. The instructions may provide information of performing any of the methods disclosed herein, whether or not the methods may be performed using only the reagents provided in the kit.

For use in the therapeutic applications described herein, kits and articles of manufacture are also described herein. In some aspects, such kits include a carrier, package, or container that is compartmentalized to receive one or more containers such as vials, tubes, and the like, each of the container(s) including one of the separate elements to be used in a method described herein. Suitable containers include, for example, bottles, vials, syringes, and test tubes. The containers can be formed from a variety of materials such as glass or plastic. The articles of manufacture provided herein contain packaging materials. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. The container(s) optionally have a sterile access port (for example the container is an intravenous solution bag or a vial having a stopper pierceable by a hypodermic injection needle). Such kits optionally comprise a composition with an identifying description or label or instructions relating to its use in the methods described herein.

A kit may include one or more additional containers, each with one or more of various materials (such as reagents, optionally in concentrated form, and/or devices) desirable from a commercial and user standpoint for use of the modulator described herein. Non-limiting examples of such materials include, but not limited to, buffers, diluents, filters, needles, syringes, carrier, package, container, vial and/or tube labels listing contents and/or instructions for use, and package inserts with instructions for use. A set of instructions will also typically be included.

In some aspects, a label is on or associated with the container. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein.

In certain aspects, a pharmaceutical composition comprising the modulators provided herein and optional additional active agent is presented in a pack or dispenser device which can contain one or more unit dosage forms. The pack can for example contain metal or plastic foil, such as a blister pack. The pack or dispenser device can be accompanied by instructions for administration. The pack or dispenser can also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, can be the labeling approved by the U.S. Food and Drug Administration for prescription drugs, or the approved product insert. Compositions containing the modulators described herein formulated in a compatible pharmaceutical carrier can also be prepared, placed in an appropriate container, and labeled for treatment of an indicated condition.

### Methods

Provided herein are methods of releases the fibroblast from quiescence stage of the cell cycle by using the modulator described herein or the synthetic polynucleic acid described herein, or the pharmaceutical composition described herein. In some instances, the modulator described herein or the synthetic polynucleic acid described herein, or the pharmaceutical composition described herein is involved in centrosome cycle. In some instances, the modulator described herein or the synthetic polynucleic acid described herein, or the pharmaceutical composition described herein is involved in microtubule organizing center organization. In some instances, the modulator described herein or the synthetic polynucleic acid described herein, or the pharmaceutical composition described herein is involved in DNA replication. In some instances, the modulator described herein or the synthetic polynucleic acid described herein, or the pharmaceutical composition described herein is involved in mitotic spindle organization. In some instances, the modulator described herein or the synthetic polynucleic acid described herein, or the pharmaceutical composition described herein is involved in mitotic sister chromatid segregation. In some instances, the modulator described herein or the synthetic polynucleic acid described herein, or the pharmaceutical composition described herein is involved in sister chromatid segregation. In some instances, the modulator described herein or the synthetic polynucleic acid described herein, or the pharmaceutical composition described herein is involved in DNA-templated DNA replication. In some instances, the modulator described herein or the synthetic polynucleic acid described herein, or the pharmaceutical composition described herein is involved in mitotic cell cycle checkpoint signaling. In some instances, the modulator described herein or the synthetic polynucleic acid described herein, or the pharmaceutical composition described herein is involved in microtubule cytoskeleton organization involved in mitosis. In some instances, the modulator described herein or the synthetic polynucleic acid described herein, or the pharmaceutical composition described herein is involved in cell cycle checkpoint signaling. In some instances, the modulator described herein or the synthetic polynucleic acid described herein, or the pharmaceutical composition described herein is involved in regulation of chromosome segregation. In some instances, the modulator described herein or the synthetic polynucleic acid described herein, or the pharmaceutical composition described herein is involved in chromosome separation. In some instances, the modulator described herein or the synthetic polynucleic acid described herein, or the pharmaceutical composition described herein is involved in spindle organization. In some instances, the modulator described herein or the synthetic polynucleic acid described herein, or the pharmaceutical composition described herein is involved in nuclear chromosome segregation. In some instances, the modulator described herein or the synthetic polynucleic acid described herein, or the pharmaceutical composition described herein is involved in regulation of chromosome organization. In some instances, the modulator described herein or the synthetic polynucleic acid described herein, or the pharmaceutical composition described herein is involved in chromosome segregation. In some instances, the modulator described herein or the synthetic polynucleic acid described herein, or the pharmaceutical composition described herein is involved in mitotic nuclear division. In some instances, the modulator described herein or the synthetic polynucleic acid described herein, or the pharmaceutical composition described herein is involved in negative regulation of cell cycle phase transition. In some instances, the modulator described herein or the synthetic polynucleic acid described herein, or the pharmaceutical composition described herein is involved in regulation of cell cycle phase transition. In some instances, the modulator described herein or the synthetic polynucleic acid described herein, or the pharmaceutical composition described herein is involved in positive regulation of cell cycle process. In some instances, the modulator described herein or the synthetic polynucleic acid described herein, or the pharmaceutical composition described herein is involved in negative regulation of cell cycle process. In some instances, the modulator described herein or the synthetic polynucleic acid described herein, or the pharmaceutical composition described herein is involved in nuclear division. In some instances, the modulator described herein or the synthetic polynucleic acid described herein, or the pharmaceutical composition described herein is involved in mitotic cell cycle phase transition. In some instances, the modulator described herein or the synthetic polynucleic acid described herein, or the pharmaceutical composition described herein is involved in meiotic cell cycle process. In some instances, the modulator described herein or the synthetic polynucleic acid described herein, or the pharmaceutical composition described herein is involved in meiotic nuclear division. In some instances, the modulator described herein or the synthetic polynucleic acid described herein, or the pharmaceutical composition described herein is involved in regulation of mitotic cell cycle phase transition. In some instances, the modulator described herein or the synthetic polynucleic acid described herein, or the pharmaceutical composition described herein is involved in meiotic cell cycle. In some instances, the modulator described herein or the synthetic polynucleic acid described herein, or the pharmaceutical composition described herein is involved in positive regulation of cell cycle. In some instances, the modulator described herein or the synthetic polynucleic acid described herein, or the pharmaceutical composition described herein is involved in regulation of mitotic cell cycle.

Also provided herein are methods of regulating inflammation with the modulator described herein or the synthetic polynucleic acid described herein, or the pharmaceutical composition described herein. In some instances, inflammation can be regulated by affecting the macrophage sub-populations. For example, in some instances, the modulator described herein or the synthetic polynucleic acid described herein, or the pharmaceutical composition described herein can decrease pro-fibrotic macrophages (M2) and/or increase non-inflammatory macrophage (M0) to reduce chronic inflammation. Inflammation can also be regulated by affecting T cell populations. For example, in some instances, the modulator described herein or the synthetic polynucleic acid described herein, or the pharmaceutical composition described herein can decrease total T cell counts, decrease activated T cells, and/or increase T regs (e.g., immunosuppressive Ctla4-expressing cells) to reduce chronic inflammation. In some instances, the modulator described herein or the synthetic polynucleic acid described herein, or the pharmaceutical composition described herein affect white blood cell counts (e.g., injury-induced leukocytes). In some instances, the modulator described herein or the synthetic polynucleic acid described herein, or the pharmaceutical composition described herein alters cell to cell communication. For example, in some instances, the modulator described herein or the synthetic polynucleic acid described herein, or the pharmaceutical composition described herein decreases communication between fibroblast and immune cell types (e.g., macrophage, T cells), compared to a control.

Further provided herein are methods of preventing pulmonary fibrosis in a subject in need thereof, the method comprising administering to the subject an effective amount of the modulator described herein or the synthetic polynucleic acid described herein, or the pharmaceutical composition described herein. Further provided herein are methods of alleviating pulmonary fibrosis in a subject in need thereof, the method comprising administering to the subject an effective amount of the modulator described herein or the synthetic polynucleic acid described herein, or the pharmaceutical composition described herein. Further provided herein are methods of treating pulmonary fibrosis in a subject in need thereof, the method comprising administering to the subject an effective amount of the modulator described herein or the synthetic polynucleic acid described herein, or the pharmaceutical composition described herein.

In some instances, the modulator described herein or the synthetic polynucleic acid described herein, or the pharmaceutical composition described herein reduces expression of fibrosis-related markers in a fibroblast affected by the pulmonary fibrosis. In some instances, the fibrosis-related markers comprise smooth muscle α actin (ACTA2), alpha 1 chain of collagen type I (COL1A1), alpha 1 chain of collagen type 3 (COL3A1), fibroblast activation protein (FAP), fibronectin 1 (FN1), periostin (POSTN), collagen triple helix repeat containing 1 (CTHRC1), alpha 1 chain of collagen type 10 (COL10A1), matrix metalloproteinase 11 (MMP11), thrombospondin 2 (THBS2), latent transforming growth factor beta binding protein 2 (LTBP2), Integrin-α11 (ITGA11), or a combination thereof.

In some instances, the modulator described herein or the synthetic polynucleic acid described herein, or the pharmaceutical composition described herein restores an expression of at least one or more genes from a set of genes comparable to a healthy cell. Accordingly, in some instances, the modulator described herein or the synthetic polynucleic acid described herein, or the pharmaceutical composition described herein restores an expression of at least 20%, 30%, 40%, 50%, 60%, 70%, 80%, or 90% of each of a set of genes comparable to a healthy cell. In some instances, the modulator described herein or the synthetic polynucleic acid described herein, or the pharmaceutical composition described herein restores an average expression of a set of genes comparable to a healthy cell. In some instances, the set of genes are aberrantly expressed in a cell or tissue affected by idiopathic pulmonary fibrosis (IPF). In some instances, “aberrantly expressed” as used herein refers to the expression of the set of genes is at least 10% 20%, 30%, 40%, 50%, 60%, 70%, 80%, or 90% higher in at least 50%, 60%, 70%, 80%, 90% of the myofibroblasts from an IPF subject when compared to a healthy subject. In some instances, “aberrantly expressed” as used herein refers to the expression of the set of genes is at least 10% 20%, 30%, 40%, 50%, 60%, 70%, 80%, or 90% lower in at least 50%, 60%, 70%, 80%, 90% of the myofibroblasts from an IPF subject when compared to a healthy subject. In some instances, the set of genes comprises one or more genes identified in Table 3.

TABLE 3
List of Genes Constituting IPF Gene Signature

| Aberrant | |
|---|---|
| expression | Gene names |
| Up- | ABCB4, ACOX2, ACTA2, ACTG2, ACTN1, ACVR1, ADAM12, ADAM19, ADAMTS10, ADAMTS16, |
| regulated | ADAMTS6, ADCY5, ADGRA2, ADORA1, ADSS1, AEBP1, AGT, AKNA, ALDH18A1, |
| | ALDH1L2, ALKAL1, ALPK2, ANGPTL2, ANK2, ANKH, ANKRD20A5P, ANO4, ANTXR1, |
| | AOPEP, APLP1, ARF4, ARHGEF25, ARMCX2, ARMH4, ASIC1, ASPN, ASS1, ASTN1, ASTN2, |
| | ATAT1, ATG13, ATP1A2, ATP2A3, AZIN2, B3GALNT2, B4GALT1, B4GALT4, BACE2, |
| | BBOF1, BCHE, BCL9, BCO2, BGN, BICC1, BLNK, BMERB1, BMP4, BMPR1B, BNC2, BOC, |
| | BRINP3, C16orf71, C1QTNF3, C1QTNF6, C1R, C1RL, C1S, C1orf122, C1orf54, C20orf85, C4A, C4B, |
| | C5orf66, C7, CABCOCO1, CACNA1C, CALB2, CALD1, CAMK1D, CAP2, CAPS, CASC15, CASTOR3, |
| | CC2D2B, CCDC144NL-AS1, CCDC170, CCDC180, CCDC40, CCDC74A, CCDC8, |
| | CCDC80, CCND2, CD24, CDH11, CDH2, CDH6, CDHR1, CDKN1A, CELSR2, CERCAM, |
| | CFAP69, CFH, CH507-9B2.1, CHD3, CHN1, CHPF, CHRDL2, CHST6, CHSY1, CILP, CILP2, |
| | CKAP4, CLCA2, CLIP3, CLMP, CLSTN2, CLSTN3, CLU, CNPY4, COL10A1, COL14A1, |
| | COL15A1, COL16A1, COL1A1, COL1A2, COL24A1, COL27A1, COL3A1, COL4A2-AS2, |
| | COL5A1, COL5A2, COL6A1, COL6A2, COL6A3, COL7A1, COL9A2, COLQ, COMP, COPZ2, |
| | CPE, CPQ, CPZ, CRACD, CREB3L2, CRLF1, CROT, CSGALNACT1, CSMD2, CST1, CTHRC1, |
| | CTSO, CUL7, CXXC5, DACH2, DACT1, DACT3, DACT3-AS1, DDIT4, DELEC1, DENND2B, |
| | DERL3, DGKA, DGKI, DMD, DMGDH, DNAH1, DNAJB13, DNAJB5, DNAJC12, |
| | DNAJC22, DNM1, DNMT3A, DPT, DPYSL3, DRC3, DRP2, DTX1, DUXAP8, DUXAP9, EAF2, |
| | ECM1, ECM2, EFCAB12, EFCAB6, EFHB, EFHC1, EFHC2, EFNA4, EFNB3, ELAPOR1, |
| | EMILIN1, ENAH, ENTPD1, ENTPD1-AS1, ENTPD7, EPHB2, EPHB3, ERGIC3, ETV6, EXTL1, |
| | EYA2, FAIM2, FAM118A, FAM131B, FAM133A, FAM13C, FAM171B, FAM198B-AS1, |
| | FAM227A, FAM227B, FAM229B, FAM66D, FAM98A, FANK1, FAP, FBLIM1, FBLN1, |
| | FBLN2, FBN1, FBXL13, FBXL22, FBXO32, FGF14, FGF18, FHL2, FILIP1L, FKBP10, FKBP11, |
| | FKBP14, FKBP7, FLRT2, FMO3, FMOD, FN1, FNDC5, FRK, FRMD5, FRMD6, FRZB, FSTL1, |
| | FUT8, FZD7, GABRB3, GAL, GALNT1, GALNT16, GAN, GAP43, GAS6-AS1, GAS7, GASK1B, |
| | GDF6, GDNF, GFUS, GLI1, GLI2, GLIS3, GLT8D1, GLT8D2, GOLGA2, GOLM1, GPC2, |
| | GPR153, GPR155, GPR173, GPR183, GPR78, GPRACR, GPX7, GPX8, GRIA3, GSEC, GSN, |
| | GXYLT2, HAPLN3, HEPH, HHAT, HIC1, HILPDA, HMCN1, HOXA3, HS3ST3A1, HSP90B1, |
| | HSPB7, HTR2B, HTRA1, HTRA3, HYDIN2, ICOSLG, IDH2, IER5L, IFITM10, IFT27, IFT43, |
| | IGDCC4, IGF1, IGFBP3, IGFBP7, IL11, IL17RD, INSYN1, INSYN2A, INTS6L, IQCK, ISLR, ITGA11, |
| | ITGA9, ITGAV, ITGB3, ITGB5, ITGBL1, ITM2C, JAK3, JAZF1, KCNA2, KCND1, KCND3, |
| | KCNH1, KCNN4, KCTD11, KDELR2, KDM5B, KIAA1755, KIF26B, KIF3C, KIF5A, KIF9-AS1, |
| | KLHL4, KSR1, LCA5L, LDB3, LDLRAD4, LEF1, LGMN, LGR4, LHPP, LINC00475, |
| | LINC00519, LINC00535, LINC00578, LINC00632, LINC00643, LINC01013, LINC01133, |
| | LINC01138, LINC01503, LINC01614, LINC01615, LINC01711, LINC01943, LINC02544, |
| | LINC02593, LINC02606, LINC02694, LINC02731, LMO4, LMOD1, LNCTAM34A, LOX, LOXL2, |
| | LOXL3, LRIG3, LRP1, LRRC15, LRRC17, LRRC27, LRRC4C, LTBP1, LTBP2, LTBP3, LUM, LUZP2, |
| | MAGED1, MAGED2, MAGED4, MAGED4B, MAGEL2, MAP3K4-AS1, MAP3K7CL, |
| | MAP6, MARCKSL1, MCAM, MDFI, MDK, MEG3, MEG8, MEGF8, MEIS3, MEOX1, |
| | MEX3A, MFAP2, MFAP4, MGP, MINAR1, MIR100HG, MIR503HG, MIR99AHG, MLLT11, |
| | MMP10, MMP11, MMP13, MMP2, MMP21, MMP3, MORC4, MPP2, MPZL1, MRAS, MRC2, |
| | MROH8, MSC, MSC-AS1, MT1F, MTHFD1L, MXRA8, MYH11, MYOCD, MYOM1, |
| | MYOSLID, NAALADL2, NAP1L3, NBAT1, NBEA, NDUFA6-DT, NEK11, NFATC4, |
| | NIPSNAP3B, NLGN2, NNMT, NOTCH3, NPAS2, NR2F1-AS1, NRP2, NYNRIN, OGN, |
| | OLFM2, OLFML2B, OLFML3, OMD, OMG, OSBPL10, OSR2, P3H1, P3H3, P3H4, P4HA3, PAK3, |
| | PAMR1, PAPPA, PAPPA-AS1, PAPPA2, PARD6G, PAX6, PBLD, PBXIP1, PCAT6, |
| | PCDH19, PCDHB12, PCDHB14, PCDHB2, PCDHB7, PCOLCE, PCSK1, PCYOX1L, PDCD4, |
| | PDGFRB, PDIA3, PDIA4, PDLIM3, PDLIM4, PDLIM7, PDZRN3, PGBD5, PGM2L1, PGM5, |
| | PKP1, PLCB4, PLEKHA6, PLN, PLOD1, PLOD2, PLP1, PLPP4, PLPP5, PLTP, PMEPA1, PNMA8A, |
| | PNMA8B, PODN, PODNL1, POGLUT2, POSTN, POU2F2, PPIB, PPIC, PPIC-AS1, |
| | PPP1R12B, PRDM1, PRDM6, PRDX4, PRG4, |
| | PRKACB, PRUNE2, PSD2, PSD3, PTCHD4, PTGFRN, PTGIS, PTHLH, PTK7, PYCR1, QPCT, |
| | RAB30, RABL2A, RAMP1, RAP2B, RASA4, RASA4B, RASD2, RASGRP3, RASL11B, RBP1, |
| | RCAN2, RCC2, RCN3, RCOR2, RERG, RGS2, RIMS2, RN7SL689P, RNF150, ROBO1, ROBO2, |
| | ROR2, RP1-152L7.5, RP1-228H13.5, RP1-37C10.7, RP11-1151B14.4, RP11-119F7.5, |
| | RP11-125O18.1, RP11-145A3.1, RP11-167N4.2, RP11-212121.3, RP11-229O3.1, RP11- |
| | 329B9.4, RP11-348N5.7, RP11-403A21.1, RP11-498C9.13, RP11-54O7.1, RP11- |
| | 54O7.16, RP11-567M16.1, RP11-624L4.1, RP11-867G23.10, RP11-893F2.5, RP11- |
| | 92C4.6, RP11-999E24.3, RP3-337O18.9, RP4-565E6.1, RP4-622L5.7, RP5-1054A22.4, |
| | RUNX1, SALL4, SAMD11, SCG2, SCG5, SCPEP1, SCRG1, SCUBE3, SDC3, SEC23A, SEC24D, |
| | SEC31A, SEL1L3, SELENOM, SEMA3C, SEPTIN6, SERPINE2, SERPINF1, SERPINI1, |
| | SESN3, SFRP4, SGCA, SGCD, SGPL1, SH3BGR, SH3PXD2A, SH3PXD2B, SH3RF3, SH3RF3-AS1, |
| | SHISAL1, SLC16A1, SLC16A2, SLC18B1, SLC1A4, SLC22A17, SLC29A3, SLC2A1, |
| | SLC2A10, SLC35F2, SLC38A4, SLC44A3-AS1, SLC46A3, SLC7A5, SLIT3, SLITRK6, |
| | SMIM10L2A, SMIM43, SMO, SMOX, SNAI2, SNCAIP, SNED1, SNRPN, SORCS2, SOX4, |
| | SPARC, SPECC1, SPON1, SPSB1, SRGAP3, SRRM3, SSC5D, SSPN, SSR4, ST8SIA2, STEAP3- |
| | AS1, STK38L, SUFU, SUGCT, SULF2, SYNDIG1, SYT12, TACR1, TAGLN, |
| | TENM3, TENM4, TEX9, TF, TGFB3, TGFBI, THBS2, THBS3, TIMP1, TMED3, TMEM117, |
| | TMEM119, TMEM132A, TMEM182, TMEM190, TMEM231, TMEM263, TMEM45A, TMEM59L, |
| | TMSB15B, TNC, TNFRSF21, TNFSF18, TNFSF4, TP53, TP53INP1, TP53TG1, TPST1, TRAF5, |
| | TRAV30, TRIM32, TRIM62, TRO, TRPS1, TRPV4, TSKU, TSPAN11, TSPAN2, TSPAN6, |
| | TTC3, TTC9, TTLL1, TTLL11, TTYH3, TUB, TWIST1, TXLNB, UCHL1, UNC5C, VASH2, VCAM1, |
| | VCAN, VCAN-AS1, VMP1, VWA1, VWCE, WHRN, WIPI1, WNT11, XBP1, XXYLT1, Xxyac- |
| | YX65C7_A.2, ZFP69B, ZFPM2, ZKSCAN7, ZMAT3, |
| | ZNF154, ZNF423, ZNF436, ZNF469, ZNF521, ZNF561-AS1, ZNF711, ZNF827, ZNF846, |
| | ZNF853 |
| Down- | AASS, ABCA3, ABHD17C, ABHD5, AC007952.5, AC009237.16, AC009237.17, AC009238.7, |
| regulated | AC009238.8, AC073130.3, AC124789.1, ACADS, ACAT1, ACER3, ACKR4, ACP3, ACVRL1, |
| | ADCY8, ADI1, ADPRH, ADRA1B, ADRB2, AFAP1L1, AFDN, AGPAT2, AGPAT3, AHNAK, |
| | AKAP1, ALCAM, ALS2CL, AMD1, AMOTL2, ANKRD29, ANKRD33B, ANXA3, APOL3, |
| | ARAP3, ARHGAP29, ARHGAP6, ARHGEF26, ARL6IP6, ATP11A, B3GALNT1, BAIAP2, BCAR3, |
| | BCAT2, BCL2L1, BLVRB, BMP2, BMPER, BNIP3, BRI3, BTBD6, C10orf95-AS1, |
| | C13orf46, C1GALT1, C1orf115, C1orf21, C20orf27, C4orf46, |
| | CAMK2D, CAPN2, CARD10, CASP4LP, CASZ1, CAV1, CAVIN2, CBR1, CC2D1B, CCBE1, |
| | CCDC68, CCDC85C, CCK, CCND3, CD274, CD36, CD47, CD55, CDAN1, CDCA7L, CDKL1, |
| | CDKL2, CDKN2AIPNL, CDKN2D, CELF2, CENPX, CEP72, CEP85, CERS2, CFL2, CGN, CHAC2, |
| | CHPT1, CHRAC1, CIT, CITED2, CLDN12, CLDN4, CLEC14A, CLEC3B, CLPP, CNTROB, |
| | CPEB2, CPNE3, CPNE8, CS, CSF2, CTC-308K20.1, CTD-2003C8.2, |
| | CTNNAL1, CTSH, CYB5A, CYC1, CYSTM1, DAGLB, DARS2, DCLRE1A, |
| | DCXR, DDX28, DENND3, DERA, DGKE, DIAPH3, DLL4, DNHD1, DNPEP, DOCK5, DOCK9, |
| | DOT1L, DSCAM, DSEL, DUSP6, E2F1, EBPL, ECHDC3, EEF1AKNMT, EFL1, EME2, EMP2, |
| | ENOSF1, EPDR1, ERBB3, ERRFI1, ESM1, EVA1A, FABP5, FAH, FAM110A, FAM111A, |
| | FAM160A1, FAM167A, FAM234B, FASN, FEM1C, FGD4, FH, FKBP4, FLII, FLRT3, FLT1, FLVCR2, |
| | FN3K, FOLR3, FZD5, GALE, GALK1, GALNT3, GATA2, GBE1, GCAT, GCDH, GCNT2, |
| | GEMIN4, GIMAP2, GIT1, GJC2, GPAT3, GPD1L, GPD2, GPER1, GPN3, GPR160, GPRC5A, GRK5, |
| | GSAP, HACD1, HAGH, HAUS4, HCG27, HIF3A, HIRIP3, HK2, HLA-E, |
| | HMSD, HNRNPF, HOPX, HPCAL1, HSBP1L1, HSDL2, HYAL2, IER2, IFIT3, IFIT5, IGF2BP2, |
| | IL15RA, IL17RE, IL18, IL6R, IMP3, IMPA1, INF2, INMT, IPO5, IRAK3, ITGA3, ITPK1, ITPR3, |
| | ITPRID2, KAT2B, KHDRBS3, KIF17, KIFC3, KLF15, KLF6, KLF9, KNSTRN, LAMA3, LDLR, |
| | LETM2, LIMD1, LINC00472, LINC00513, LINC01224, LINC01273, LINC02185, LIPH, |
| | LPCAT1, LRRN4, LSM6, LSS, MACIR, MAOA, MAP3K6, MAP4K2, MAPK13, MBP, MCCC1, MCFD2, |
| | MFSD13A, MFSD2A, MGLL, MGST1, MIDEAS, MLPH, MLX, MME, MRPL14, MTMR12, |
| | MYO1C, NADK, NAGS, NBEAL2, NCAPH2, NCKAP5, NCKAP5-AS2, |
| | NEDD4, NEDD4L, NEMP1, NFKBIA, NHSL1, NIBAN2, NIPA1, NOTCH1, NPC1, |
| | NR3C2, NRGN, NTHL1, NTNG1, NUDCD1, NUDT15, NUDT16L2P, NUP58, OCLN, OGDH, |
| | OSGIN1, OSTF1, P2RY1, P3H2, PAK4, PALB2, PALM2AKAP2, PAQR5, PARP12, PCID2, |
| | PCYT2, PDE12, PDE4DIPP2, PDP2, PEAR1, PECR, PEG10, PGAM5, PGAP6, PHLPP1, PI4K2B, |
| | PIEZO1, PITPNM2, PITRM1, PKDCC, PKN1, PKN3, PLAAT3, PLEKHJ1, PLEKHM1, PLIN2, |
| | PMM1, PODXL, POLA2, POLE, POPDC3, PPA1, PPARG, PPFIBP1, PPL, PPM1F, PPP1R15A, |
| | PPP2R5A, PREX1, PRKAR2B, PRKCE, PRKCQ- |
| | AS1, PRKG2, PRLR, PROSER2, PRRG4, PTPRQ, PVR, PYCARD, QDPR, RAB11FIP1, RAB17, |
| | RAB20, RAB32, RAB3D, RAP1GAP2, RAPGEF4, RASIP1, RGMB, RHBDF1, RHOF, RIPOR1, |
| | RNH1, ROBO4, RP1-267D11.6, RP11-2N1.2, RP11-432J24.5, RP11-61J19.5, RP11- |
| | 63G10.4, RP11-867O8.11, RP11-93B14.10, RP11-96C23.10, RP11-96C23.5, RP3- |
| | 331H24.6, RP3-342P20.2, RP3-403A15.5, RPS6KA1, |
| | RPS6KA2, RTN4, RTTN, S100A3, SASH1, SCML1, SEMA3E, SFTA1P, SGO1, SH2D5, |
| | SH3RF1, SHANK2, SHMT1, SHROOM1, SIGIRR, SLC14A1, SLC16A12, SLC25A24, SLC25A25, |
| | SLC25A4, SLC25A5, SLC44A2, SLC51B, SLC66A1L, SLCO4A1, SLITRK2, SMAGP, SMC4, |
| | SMIM29, SMURF2, SPC24, SPC25, SPRY2, SPRY4, SPRY4-AS1, |
| | SPTBN1, SPTLC3, SQOR, SSTR1, STAC, STARD3NL, STARD7, |
| | STARD8, STBD1, STRADB, STX11, STX3, SUN2, SVIP, SYNPO2L, SYTL4, TACC2, TAOK3, |
| | TBC1D4, TBRG4, TBX2-AS1, TCF21, TDRD7, TEAD4, TEC, TENT5B, TFPI, TGFBR3, |
| | TJP2, TLR3, TM4SF4, |
| | TMBIM1, TMEM106C, TMEM192, TMEM200B, TMEM245, TMEM53, TMEM62, TMPO, |
| | TMTC1, TNNT1, TOM1L1, TOR4A, TOX, TOX2, TPRN, TPST2, TRHDE-AS1, |
| | TRIM25, TRIM58, TRNP1, TSPAN4, |
| | TTC39A, TTLL12, TTN, TUBGCP3, TXNRD1, TXNRD2, UNC13B, USP1, USP13, USP31, |
| | USP53, USP54, UTP18, UTRN, VEPH1, VSIG10, VSIR, WDR5, WNT3, WWC1, WWC3, ZBTB42, |
| | ZC3H12C, ZDHHC12, ZDHHC14, ZDHHC7, ZNF185, ZNF726P1, ZNF792, ZNF823 |

In some instances, the modulator described herein, the synthetic polynucleic acid described herein, or the pharmaceutical composition described herein effectively alleviates or treats pulmonary fibrosis by increasing non-inflammatory macrophage (M0) counts and/or decreasing pro-fibrotic macrophages (M2). In some instances, the modulator described herein or the synthetic polynucleic acid described herein, or the pharmaceutical composition described herein effectively alleviates or treats pulmonary fibrosis by reducing total T cell counts and activated T cell, and/or by increasing immunosuppressive Ctla4-expressing cells. In some instances, the modulator described herein, the synthetic polynucleic acid described herein, or the pharmaceutical composition described herein effectively alleviates or treats pulmonary fibrosis by decreasing total lung injury-induced leukocytes in the BAL (Bronchioalveolar Lavage). In some instances, the modulator described herein, the synthetic polynucleic acid described herein, or the pharmaceutical composition described herein effectively alleviates or treats pulmonary fibrosis by decreasing the cell-cell communication between fibroblasts and immune cells (e.g., illustrated by ligand-receptor pairs between fibroblasts and immune cells).

In some instances, the modulator described herein or the synthetic polynucleic acid described herein, or the pharmaceutical composition described herein delays onset, alleviates the intensity or frequency of, or prevents progression of a symptom of a pulmonary fibrosis in the subject. In some instances, the modulator described herein or the synthetic polynucleic acid described herein, or the pharmaceutical composition described herein prevents or treats a symptom of a pulmonary fibrosis in the subject. In some instances, the modulator described herein or the synthetic polynucleic acid described herein, or the pharmaceutical composition described herein reverse the disease prognosis of the pulmonary fibrosis.

In some instances, the modulator described herein or the synthetic polynucleic acid described herein, or the pharmaceutical composition described herein modulates an immune response in or around the tissue or cells affected by fibrosis (e.g., pulmonary fibrosis). In some instances, the modulator described herein or the synthetic polynucleic acid described herein, or the pharmaceutical composition described herein increases or facilitates the immune response in or around the tissue or cells affected by fibrosis (e.g., pulmonary fibrosis). In some instances, the modulator described herein or the synthetic polynucleic acid described herein, or the pharmaceutical composition described herein suppresses or reverses the immune response in or around the tissue or cells affected by fibrosis (e.g., pulmonary fibrosis). In some instances, the modulator described herein or the synthetic polynucleic acid described herein, or the pharmaceutical composition described herein modulates the immune response targeting a specific cell type in the tissue affected by fibrosis. In some instances, the immune response comprises an increase of expression and/or activity of MCP-1, an increase of expression and/or activity of IP-10, a decrease of expression and/or activity of IL-10, or a combination thereof.

In some instances, the modulator described herein or the synthetic polynucleic acid described herein, or the pharmaceutical composition described herein modulates the balance between immune response and cell state transitions/maintenance among one or more cell types in the tissue affected by fibrosis (e.g., pulmonary fibrosis). In some instances, the modulator described herein or the synthetic polynucleic acid described herein, or the pharmaceutical composition described herein modulates the balance between proliferation and extracellular matrix synthesis among one or more cell types in the tissue affected by fibrosis (e.g., pulmonary fibrosis). For example, in some instances, the modulator described herein or the synthetic polynucleic acid described herein, or the pharmaceutical composition described herein prevents, reduces, alleviates, or reverses the generation, proliferation, or activity of myofibroblast associated with onset, development, or progression of fibrosis, and facilitates, increases, or induces the immune responses or inflammatory responses/reactions in the same tissue such that the balance is moving towards one direction (e.g., immune response) over the other (e.g., generation of myofibroblast). In other instances, the modulator described herein or the synthetic polynucleic acid described herein, or the pharmaceutical composition described herein ablates a subpopulation of myofibroblasts that are associated with onset, development, or progression of fibrosis.

In some instances, the modulator described herein or the synthetic polynucleic acid described herein, or the pharmaceutical composition described herein is encapsulated in a liposome or coupled with a nanoparticle. In some instances, the modulator described herein or the synthetic polynucleic acid described herein, or the pharmaceutical composition described herein is encoded by a transgene in an expression vector. In some instances, the modulator or the synthetic polynucleic acid described herein is encapsulated in an extracellular vesicle.

In some instances, the administering is performed intratracheally, orally, nasally, intravenously, intraperitoneally, or intramuscularly. In some instances, the administering is a targeted delivery to a lung tissue of the subject. In some instances, the administering is in a form of aerosol. In some instances, the aerodynamic diameter of particles of the modulator the synthetic polynucleic acid disclosed herein is less than 10 μm. In some instances, the aerodynamic diameter of particles of the modulator the synthetic polynucleic acid disclosed herein is less than 5 μm. In some instances, the aerodynamic diameter of particles of the modulator the synthetic polynucleic acid disclosed herein is less than 3 μm.

For delivery to the target cell or tissue (e.g., lung tissue), the modulator or the synthetic polynucleic acid described herein can non-covalently bind an excipient to form a complex. The excipient can be used to alter biodistribution after delivery, to enhance uptake, to increase half-life or stability of the strands in the modulator described herein (e.g., improve nuclease resistance), and/or to increase targeting to a particular cell or tissue type. Exemplary excipients include but are not limited to a condensing agent (e.g., an agent capable of attracting or binding a nucleic acid through ionic or electrostatic interactions); a fusogenic agent (e.g., an agent capable of fusing and/or being transported through a cell membrane); a protein to target a particular cell or tissue type (e.g., thyrotropin, melanotropin, lectin, glycoprotein, surfactant protein A, or any other protein); a lipid; a lipopolysaccharide; a lipid micelle or a liposome (e.g., formed from phospholipids, such as phosphotidylcholine, fatty acids, glycolipids, ceramides, glycerides, cholesterols, or any combination thereof); a nanoparticle (e.g., silica, lipid, carbohydrate, or other pharmaceutically-acceptable polymer nanoparticle); a polyplex formed from cationic polymers and an anionic agent (e.g., a CRO), where exemplary cationic polymers include but are not limited to polyamines (e.g., polylysine, polyarginine, polyamidoamine, and polyethylene imine); cholesterol; a dendrimer (e.g., a polyamidoamine (PAMAM) dendrimer); a serum protein (e.g., human serum albumin (HSA) or low-density lipoprotein (LDL)); a carbohydrate (e.g., dextran, pullulan, chitin, chitosan, inulin, cyclodextrin, or hyaluronic acid); a lipid; a synthetic polymer, (e.g., polylysine (PLL), polyethylenimine, poly-L-aspartic acid, poly-L-glutamic acid, styrene-maleic acid anhydride copolymer, poly(L-lactide-co-glycolic) copolymer, divinyl ether-maleic anhydride copolymer, N-(2-hydroxypropyl) methacrylamide copolymer (HMPA), polyethylene glycol (PEG), polyvinyl alcohol (PVA), polyurethane, poly(2-ethylacrylic acid), N-isopropylacrylamide polymer, pseudopeptide-polyamine, peptidomimetic polyamine, or polyamine); a cationic moiety (e.g., cationic lipid, cationic porphyrin, quaternary salt of a polyamine, or alpha helical peptide); a multivalent sugar (e.g., multivalent lactose, multivalent galactose, N-acetyl-galactosamine, N-acetyl-glucosamine, multivalent mannose, or multivalent fucose); a vitamin (e.g., vitamin A, vitamin E, vitamin K, vitamin B, folic acid, vitamin B12, riboflavin, biotin, or pyridoxal); a cofactor; or a drug to disrupt cellular cytoskeleton to increase uptake (e.g., taxol, vincristine, vinblastine, cytochalasin, nocodazole, japlakinolide, latrunculin A, phalloidin, swinholide A, indanocine, or myoservin).

In some aspects, the administering is a targeted delivery to a lung tissue of the subject. In some instances, the targeted delivery is via a local application. In some instances, the targeted delivery is via one or more specific binding moieties that target the lung tissue.

Also provided herein are methods of reprogramming a myofibroblast into a cell type specific or predominates an early stage of lung development. Also provided herein are methods of reprogramming a myofibroblast into an early fibroblast precursor cell. In some instances, the early fibroblast precursor cell is a mesothelial cell, an early fibroblast cell, a mid fibroblast cell, or a mesenchymal cell. Also provided herein are methods of reprogramming a myofibroblast into a cell that is Top2a, Cenpa or ki67 positive. In some instances, the cell that is Top2a, Cenpa or ki67 positive is a mesenchymal progenitor cell. In some instances, the mesenchymal progenitor cell is a bronchioalveolar stem cells (BASC), an endothelial progenitor cell, or a fibroblast progenitor cell. Also provided herein are methods of reprogramming a myofibroblast into a proliferating fibroblast. In some instances, the proliferating fibroblast displays a unique transcriptional profile that is discussed in Example 6. In some instances, the methods comprise contacting the myofibroblast with an effective amount of the modulators described herein or the pharmaceutical composition described herein. In some instances, the activity of Ebf1 is decreased. In some instances, activity of Sox2, a lung regeneration transcription factor, is increased. In some instances, the expression level of one or more gene from the proliferating gene signature described in Example 6 is upregulated, which also indicates a potential cellular reprogramming occurred. In some instances, the methods of reprogramming into a progenitor cell without further differentiating to a differentiated cell type requires continuous administration of the modulator described herein or the synthetic polynucleic acid described herein, or the pharmaceutical composition described herein.

In some instances, the modulator described herein or the synthetic polynucleic acid described herein, or the pharmaceutical composition described herein is used to reprogram a myofibroblast. In some instances, the modulator described herein or the synthetic polynucleic acid described herein, or the pharmaceutical composition described herein is used to reprogram a fibroblast (e.g., early fibroblast, middle fibroblast, fully developed fibroblast). In some instances, the modulator described herein or the synthetic polynucleic acid described herein, or the pharmaceutical composition described herein is used to reprogram early fibroblast precursor cells. In some instances, the modulator described herein or the synthetic polynucleic acid described herein, or the pharmaceutical composition described herein is used to reprogram mesenchymal cells. In some instances, the modulator described herein or the synthetic polynucleic acid described herein, or the pharmaceutical composition described herein is used to reprogram mesenchymal progenitor cells. In some instances, the modulator described herein or the synthetic polynucleic acid described herein, or the pharmaceutical composition described herein is used to reprogram mesothelial cells. In some instances, the modulator described herein or the synthetic polynucleic acid described herein, or the pharmaceutical composition described herein is used to reprogram smooth muscle cells (e.g., airway smooth muscle cells, vascular smooth muscle cells). In some instances, the modulator described herein or the synthetic polynucleic acid described herein, or the pharmaceutical composition described herein is used to reprogram pericytes. In some instances, the modulator described herein or the synthetic polynucleic acid described herein, or the pharmaceutical composition described herein is used to reprogram alveolar epithelial type II (AT2) cells. In some instances, the modulator described herein or the synthetic polynucleic acid described herein, or the pharmaceutical composition described herein is used to reprogram alveolar epithelial type I (AT1) cells.

In some instances, the modulator described herein or the synthetic polynucleic acid described herein, or the pharmaceutical composition described herein is used to reprogram a cell (e.g., differentiated cell, myofibroblast) into an earlier cell state. In some instances, the modulator described herein or the synthetic polynucleic acid described herein, or the pharmaceutical composition described herein is used to reprogram a cell (e.g., differentiated cell, myofibroblast) into a fibroblast (e.g., early fibroblast, middle fibroblast, fully developed fibroblast). In some instances, the modulator described herein or the synthetic polynucleic acid described herein, or the pharmaceutical composition described herein is used to reprogram a cell (e.g., differentiated cell, myofibroblast) into an early fibroblast precursor cell. In some instances, the modulator described herein or the synthetic polynucleic acid described herein, or the pharmaceutical composition described herein is used to reprogram a cell (e.g., differentiated cell) into a myofibroblast. In some instances, the modulator described herein or the synthetic polynucleic acid described herein, or the pharmaceutical composition described herein is used to reprogram a cell (e.g., differentiated cell, myofibroblast) into a mesenchymal cell. In some instances, the modulator described herein or the synthetic polynucleic acid described herein, or the pharmaceutical composition described herein is used to reprogram a cell (e.g., differentiated cell, myofibroblast) into a mesenchymal progenitor cell. In some instances, the modulator described herein or the synthetic polynucleic acid described herein, or the pharmaceutical composition described herein is used to reprogram a cell (e.g., differentiated cell, myofibroblast) into endothelial progenitors. In some instances, the modulator described herein or the synthetic polynucleic acid described herein, or the pharmaceutical composition described herein is used to reprogram a cell (e.g., differentiated cell, myofibroblast) into a fibroblast progenitor. In some instances, the modulator described herein or the synthetic polynucleic acid described herein, or the pharmaceutical composition described herein is used to reprogram a cell (e.g., differentiated cell, myofibroblast) into a stem cell (e.g., bronchioalveolar stem cell). In some instances, the modulator described herein or the synthetic polynucleic acid described herein, or the pharmaceutical composition described herein is used to reprogram a cell (e.g., differentiated cell, myofibroblast) into a mesothelial cell. In some instances, the modulator described herein or the synthetic polynucleic acid described herein, or the pharmaceutical composition described herein is used to reprogram a cell (e.g., differentiated cell, myofibroblast) into a smooth muscle cell (e.g., airway smooth muscle cell, vascular smooth muscle cell). In some instances, the modulator described herein or the synthetic polynucleic acid described herein, or the pharmaceutical composition described herein is used to reprogram a cell (e.g., differentiated cell, myofibroblast) into a pericyte. In some Instances, the modulator described herein or the synthetic polynucleic acid described herein, or the pharmaceutical composition described herein is used to reprogram a cell (e.g., differentiated cell, myofibroblast) into an AT2 cell. In some instances, the modulator described herein or the synthetic polynucleic acid described herein, or the pharmaceutical composition described herein is used to reprogram a cell (e.g., differentiated cell, myofibroblast) into an AT1 cell.

In some instances, the modulator described herein or the synthetic polynucleic acid described herein, or the pharmaceutical composition described herein is used to reprogram a cell (e.g., differentiated cell, myofibroblast) into a mesenchymal progenitor cell. In some instances, the modulator described herein or the synthetic polynucleic acid described herein, or the pharmaceutical composition described herein is used to reprogram a cell (e.g., differentiated cell, myofibroblast) into a cell expressing higher levels of Ki67, Top2a, Cenpa, compared to a previous cell state.

In some instances, the modulator described herein or the synthetic polynucleic acid described herein, or the pharmaceutical composition described herein is used to reprogram a cell (e.g., differentiated cell, myofibroblast) into a younger cell state or with decreased cell senescence. In some instances, the modulator described herein or the synthetic polynucleic acid described herein, or the pharmaceutical composition described herein is used to reprogram a cell (e.g., differentiated cell, myofibroblast) into a cell expressing one or more genes associated with a young signature compared to a cell without the modulator described herein or the synthetic polynucleic acid described herein, or the pharmaceutical composition described herein. A young signature can comprise of genes involved in cell cycle, cytoskeleton, ubiquitin-like protein conjugation, DNA damage, mitochondrion, mitochondrial inner membrane, DNA repair, mitochondrial translation, or a combination thereof. In some instances, a young signature can comprise of genes associated with proliferating cells (e.g., proliferating basal cells, macrophage and natural killer cells). In some instances, the modulator described herein or the synthetic polynucleic acid described herein, or the pharmaceutical composition described herein is used to reprogram a cell (e.g., differentiated cell, myofibroblast) into a cell expressing less genes associated with an old signature compared to a cell without the modulator described herein or the synthetic polynucleic acid described herein, or the pharmaceutical composition described herein. An old signature can comprise of genes involved in cadherin, cell adhesion, nervous system development, stress response, tyrosine-protein kinase, extracellular matrix, heat shock protein 70 family, growth factor, or a combination thereof. In some instances, an old signature can comprise of genes associated with myofibroblasts.

In some instances, the modulator described herein or the synthetic polynucleic acid described herein, or the pharmaceutical composition described herein contributes to rejuvenation, reversal of aging, or reduction of aging rates. Rejuvenation, reversal of aging, or reduction of aging rates can be achieved through cellular reprogramming (e.g., partial or full cellular reprogramming). In some instances, the modulator described herein or the synthetic polynucleic acid described herein, or the pharmaceutical composition described herein is involved in cellular reprogramming. In some instances, the modulator described herein or the synthetic polynucleic acid described herein, or the pharmaceutical composition described herein is involved in partial cellular reprogramming. Reprogramming can result in expression of particular genes by the cells, the expression of which further contributes to reprogramming. Reprogramming can result in chromatin rearrangement and/or change in chromatin accessibility. In some instances, the modulator described herein or the synthetic polynucleic acid described herein, or the pharmaceutical composition described herein is involved in epigenomic reprogramming.

Also provided herein are methods of slowing or reversing aging of a pulmonary tissue in a subject, the method comprising administering to the subject an effective amount of the modulators described herein or the pharmaceutical composition described herein. In some instances, an upregulated proteostasis is observed in the pulmonary tissue. In some instances, an expression of Zmpste24 or Lamp2 is increased in the pulmonary tissue. In some instances, an upregulated macro-autophagy is observed in the pulmonary tissue. In some instances, the expression of Atg5 or Atg7 is increased in the pulmonary tissue. In some instances, a more active telomerase is observed in the pulmonary tissue. In some instances, an expression of Tert or Terc is increased in the pulmonary tissue. In some instances, an age-related inflammation or an age-related tissue repair capability is improved in the pulmonary tissue. In some instances, more M0 macrophages or fewer profibrotic M2 macrophages are observed in the pulmonary tissue. In some instances, more M0 macrophages or fewer profibrotic M2 macrophages are observed in bronchoalveolar lavage (BAL) of the subject. In some instances, fewer white blood cells or fewer macrophages are observed in a bronchioalveolar lavage of the subject. In some instances, a number of total T cells or activated T cells is decreased in the pulmonary tissue. In some instances, a number of immunosuppressive Ctla4-expressing cells is increased in the pulmonary tissue. In some instances, a cell-cell communication between a fibroblast and an immune cell (e.g., a T cell, an M0, M1, or M2 macrophage) is decreased in the pulmonary tissue. In some instances, a cell-cell communication between a fibroblast and an immune cell (e.g., a T cell, an M0, M1, or M2 macrophage) is decreased in blood of the subject. In some instances, a cell-cell communication between a fibroblast and an immune cell (e.g., a T cell, an M0, M1, or M2 macrophage) is decreased in bronchoalveolar lavage (BAL) of the subject. In some instances, an activity of sry-related HMG box 2 (Sox2) is increased. In some instances, an age-related epigenetic alteration (e.g., an altered chromatin plasticity and an altered histone modification) is reversed. In some instances, cellular senescence measured by Imna nuclear lamina gene is reversed. In some instances, an age-associated alteration in PI3K-AKT pathway is reversed. In some instances, an age-associated alteration in Ras-MEK-ERK pathways is reversed. In some instances, an expression of a gene specific to a young population that is selected from a supplementary table 7 of Chow et al in Example 5 is increased in the pulmonary tissue. In some instances, an expression of a gene specific to an old population that is selected from a supplementary table 7 of Chow et al in Example 5 is decreased in the pulmonary tissue. In some instances, an expression of a gene specific to a proliferating fibroblast as referred to in Example 6 is increased in the pulmonary tissue. In some instances, chromatin plasticity is enhanced (measured by e.g., increase in insulation score by micro-C assay). In some instances, the reversing aging is reflected as an emergence of a younger cell type after administering the modulator described herein or the synthetic polynucleic acid described herein, or the pharmaceutical composition.

In some instances, the modulator described herein or the synthetic polynucleic acid described herein, or the pharmaceutical composition described herein affects functions associated with aging. For example, in some instances, the modulator described herein or the synthetic polynucleic acid described herein, or the pharmaceutical composition described herein increases proteostasis, autophagy, and/or telomerase, compared to a control (e.g., without the modulator described herein or the synthetic polynucleic acid described herein, or the pharmaceutical composition described herein). Proteostasis or loss of protein homeostasis can be a feature of aging. Re-establishing proteostasis can attenuate pulmonary fibrosis. In some instances, the modulator described herein or the synthetic polynucleic acid described herein, or the pharmaceutical composition described herein increase genes associated with proteostasis, such as Zmpste24 or Lamp2. In some instances, the modulator described herein or the synthetic polynucleic acid described herein, or the pharmaceutical composition described herein increase genes associated with autophagy, such as Atg5 or Atg7. In some instances, the modulator described herein or the synthetic polynucleic acid described herein, or the pharmaceutical composition described herein increase genes associated with telomerase, such as Tert or Terc.

Various methods can be used to evaluate the efficacy of administration of the modulators described herein or the pharmaceutical composition described herein to slow or reverse aging of a pulmonary tissue. In some instances, hallmarks of aging, including but not limited to, chronic inflammation, altered intercellular communication, emergence and expansion of progenitors, lung transcriptomic aging clock, telomere attrition, loss of proteostasis, and disabled macroautophagy, are evaluated. In some instances, genomic DNA is extracted. In some instances, DNA methylation is profiled. In some instances, histone modifiers (e.g., Sirt1, Sirt6 and Sirt7) are measured. In some instances, snATAC-seq is performed. In some instances, Micro-C is performed. In some instances, Cut&Run is performed. In some instances, metabolomic readouts are measured.

Also provided herein are methods for diagnosing or monitoring pulmonary fibrosis in a subject, the method comprising: (a) obtaining a biological sample derived from the subject; (b) detecting an amount and/or an activity of a plurality of biomarkers from the biological sample, wherein the plurality of biomarkers comprise a transposable element transcript transcribed from a transposable element, wherein the transposable element comprises a sequence that is at least 60%, at least 70%, at least 80%, at least 90%, or at least 95% identical to at least a portion of a sequence selected from of SEQ ID NOs: 4, 7-11, and 14-121; and (c) diagnosing the subject with pulmonary fibrosis or to have a high/higher chance to contract pulmonary fibrosis if the amount and/or the activity of the plurality of biomarkers is at least 50%, at least 60%, at least 70%, at least 80%, at least 90% higher when compared to a control. In some instances, the pulmonary fibrosis is idiopathic pulmonary fibrosis (IPF). In some instances, the sample is an alveolar type 2 cell (AT2 cell) or a lung fibroblast. In some instances, the methods further comprise d) administering to the subject the modulator described herein or the pharmaceutical composition described herein. In some instances, the modulator described herein or the pharmaceutical composition described herein is administered intratracheally, oropharyngeally, orally, nasally, intravenously, intraperitoneally, or intramuscularly. In some instances, the plurality of biomarkers comprise one or more fibrosis-related markers (e.g., smooth muscle α actin (ACTA2), alpha 1 chain of collagen type I (COL1A1), alpha 1 chain of collagen type 3 (COL3A1), fibroblast activation protein (FAP), fibronectin 1 (FN1), periostin (POSTN), collagen triple helix repeat containing 1 (CTHRC1), alpha 1 chain of collagen type 10 (COL10A1), matrix metalloproteinase 11 (MMP11), thrombospondin 2 (THBS2), latent transforming growth factor beta binding protein 2 (LTBP2), Integrin-α11 (ITGA11), TERT, or a combination thereof, or one or more genes identified in Table 3 and Table 5). In some instances, the detecting comprises using S1 nuclease protection assay, microarray analysis, polymerase chain reaction (PCR), hybridization technologies, reverse transcriptase polymerase chain reaction (RT-PCR), Northern blot, serial analysis of gene expression (SAGE), immunoassay, and/or mass spectrometry, or any sequencing-based methods known in the art. In some instances, exemplary any sequencing-based methods include but are not limited to bulk RNA sequencing, single cell or single nucleus RNA sequencing, DNA sequencing, DNA methylation profiling (e.g., for example, average % methylated CpG along the transposable element locus or the methylation status of specific CpGs within the transposable element), sc- or snATAC-seq or bulk ATAC-seq, DNase I assay for accessibility, CUT&RUN or ChIP-seq for specific histone marks (e.g., H3K27ac, H4K16ac, H3K9me3, H3K27me3, H3K4me1, H3K4me3, etc.).

Also provided herein are methods of predicting severity and progression of pulmonary fibrosis in a subject, the method comprising: (a) obtaining a biological sample derived from the subject; (b) detecting an amount and/or an activity of a plurality of biomarkers from the biological sample, wherein the plurality of biomarkers comprise a transposable element transcript transcribed from a transposable element, wherein the transposable element comprises a sequence that is at least 60%, at least 70%, at least 80%, at least 90%, or at least 95% identical to at least a portion of a sequence selected from of SEQ ID NOs: 4, 7-11, and 14-121; and (c) predicting the subject to have a more severe or a progression of pulmonary fibrosis if the amount and/or the activity of the plurality of biomarkers is at least 50%, at least 60%, at least 70%, at least 80%, at least 90% higher when compared to a control. In some instances, the pulmonary fibrosis is idiopathic pulmonary fibrosis (IPF). In some instances, the sample is an alveolar type 2 cell (AT2 cell) or a lung fibroblast. In some instances, the methods further comprise d) administering to the subject the modulator described herein or the pharmaceutical composition described herein. In some instances, the modulator described herein or the pharmaceutical composition described herein is administered intratracheally, oropharyngeally, orally, nasally, intravenously, intraperitoneally, or intramuscularly. In some instances, the plurality of biomarkers comprise one or more fibrosis-related markers (e.g., smooth muscle α actin (ACTA2), alpha 1 chain of collagen type I (COL1A1), alpha 1 chain of collagen type 3 (COL3A1), fibroblast activation protein (FAP), fibronectin 1 (FN1), periostin (POSTN), collagen triple helix repeat containing 1 (CTHRC1), alpha 1 chain of collagen type 10 (COL10A1), matrix metalloproteinase 11 (MMP11), thrombospondin 2 (THBS2), latent transforming growth factor beta binding protein 2 (LTBP2), Integrin-α11 (ITGA11), TERT, or a combination thereof, or one or more genes identified in Table 3 and Table 5). In some instances, the detecting comprises using S1 nuclease protection assay, microarray analysis, polymerase chain reaction (PCR), hybridization technologies, reverse transcriptase polymerase chain reaction (RT-PCR), Northern blot, serial analysis of gene expression (SAGE), immunoassay, and/or mass spectrometry, or any sequencing-based methods known in the art. In some instances, exemplary any sequencing-based methods include but are not limited to bulk RNA sequencing, single cell or single nucleus RNA sequencing, DNA sequencing, DNA methylation profiling (e.g., for example, average % methylated CpG along the transposable element locus or the methylation status of specific CpGs within the transposable element), sc- or snATAC-seq or bulk ATAC-seq, DNase I assay for accessibility, CUT&RUN or ChIP-seq for specific histone marks (e.g., H3K27ac, H4K16ac, H3K9me3, H3K27me3, H3K4me1, H3K4me3, etc.).

Also provided herein are methods for monitoring an efficacy of a pulmonary therapy in a subject, the method comprising: (a) obtaining a biological sample derived from the subject before and after the pulmonary therapy; (b) detecting an amount and/or an activity of a plurality of biomarkers from the biological sample, wherein the plurality of biomarkers comprise a transposable element transcript transcribed from a transposable element, wherein the transposable element comprises a sequence that is at least 60%, at least 70%, at least 80%, at least 90%, or at least 95% identical to at least a portion of a sequence selected from of SEQ ID NOs: 4, 7-11, and 14-121; and (c) concluding the pulmonary therapy is effective if the amount and/or the activity of the plurality of biomarkers after the pulmonary therapy is at least 50%, at least 60%, at least 70%, at least 80%, at least 90% lower when compared to the amount and/or the activity of the plurality of biomarkers before the pulmonary therapy. In some instances, the sample is an alveolar type 2 cell (AT2 cell) or a lung fibroblast. In some instances, if the pulmonary therapy is not effective as expected, the methods further comprise d) administering to the subject the modulator described herein or the pharmaceutical composition described herein. In some instances, the modulator described herein or the pharmaceutical composition described herein is administered intratracheally, oropharyngeally, orally, nasally, intravenously, intraperitoneally, or intramuscularly. In some instances, the plurality of biomarkers comprise one or more fibrosis-related markers (e.g., smooth muscle α actin (ACTA2), alpha 1 chain of collagen type I (COL1A1), alpha 1 chain of collagen type 3 (COL3A1), fibroblast activation protein (FAP), fibronectin 1 (FN1), periostin (POSTN), collagen triple helix repeat containing 1 (CTHRC1), alpha 1 chain of collagen type 10 (COL10A1), matrix metalloproteinase 11 (MMP11), thrombospondin 2 (THBS2), latent transforming growth factor beta binding protein 2 (LTBP2), Integrin-α11 (ITGA11), TERT, or a combination thereof, or one or more genes identified in Table 3 and Table 5). In some instances, the detecting comprises using S1 nuclease protection assay, microarray analysis, polymerase chain reaction (PCR), hybridization technologies, reverse transcriptase polymerase chain reaction (RT-PCR), Northern blot, serial analysis of gene expression (SAGE), immunoassay, and/or mass spectrometry, or any sequencing-based methods known in the art. In some instances, exemplary any sequencing-based methods include but are not limited to bulk RNA sequencing, single cell or single nucleus RNA sequencing, DNA sequencing, DNA methylation profiling (e.g., for example, average % methylated CpG along the transposable element locus or the methylation status of specific CpGs within the transposable element), sc- or snATAC-seq or bulk ATAC-seq, DNase I assay for accessibility, CUT&RUN or ChIP-seq for specific histone marks (e.g., H3K27ac, H4K16ac, H3K9me3, H3K27me3, H3K4me1, H3K4me3, etc.).

Also provided herein are methods for evaluating or monitoring pulmonary tissue regeneration in a subject, the method comprising: (a) obtaining a biological sample derived from the subject at an earlier time point and a later time point; (b) detecting an amount and/or an activity of a plurality of biomarkers from the biological sample, wherein the plurality of biomarkers comprise a transposable element transcript transcribed from a transposable element, wherein the transposable element comprises a sequence that is at least 60%, at least 70%, at least 80%, at least 90%, or at least 95% identical to at least a portion of a sequence selected from of SEQ ID NOs: 4, 7-11, and 14-121; and (c) concluding with a positive pulmonary tissue regeneration if the amount and/or the activity of the plurality of biomarkers at the later time point is at least 50%, at least 60%, at least 70%, at least 80%, at least 90% lower when compared to the amount and/or the activity of the plurality of biomarkers at the earlier time point. In some instances, the sample is an alveolar type 2 cell (AT2 cell) or a lung fibroblast. In some instances, if the pulmonary tissue regeneration is not as expected (e.g., not enough regeneration), the methods further comprise d) administering to the subject the modulator described herein or the pharmaceutical composition described herein. In some instances, the modulator described herein or the pharmaceutical composition described herein is administered intratracheally, oropharyngeally, orally, nasally, intravenously, intraperitoneally, or intramuscularly. In some instances, the plurality of biomarkers comprise one or more fibrosis-related markers (e.g., smooth muscle α actin (ACTA2), alpha 1 chain of collagen type I (COL1A1), alpha 1 chain of collagen type 3 (COL3A1), fibroblast activation protein (FAP), fibronectin 1 (FN1), periostin (POSTN), collagen triple helix repeat containing 1 (CTHRC1), alpha 1 chain of collagen type 10 (COL10A1), matrix metalloproteinase 11 (MMP11), thrombospondin 2 (THBS2), latent transforming growth factor beta binding protein 2 (LTBP2), Integrin-α11 (ITGA11), TERT, or a combination thereof, or one or more genes identified in Table 3 and Table 5). In some instances, the detecting comprises using S1 nuclease protection assay, microarray analysis, polymerase chain reaction (PCR), hybridization technologies, reverse transcriptase polymerase chain reaction (RT-PCR), Northern blot, serial analysis of gene expression (SAGE), immunoassay, and/or mass spectrometry, or any sequencing-based methods known in the art. In some instances, exemplary any sequencing-based methods include but are not limited to bulk RNA sequencing, single cell or single nucleus RNA sequencing, DNA sequencing, DNA methylation profiling (e.g., for example, average % methylated CpG along the transposable element locus or the methylation status of specific CpGs within the transposable element), sc- or snATAC-seq or bulk ATAC-seq, DNase I assay for accessibility, CUT&RUN or ChIP-seq for specific histone marks (e.g., H3K27ac, H4K16ac, H3K9me3, H3K27me3, H3K4me1, H3K4me3, etc.).

Also provided herein are methods for evaluating or monitoring efficacy of a treatment to induce pulmonary reprogramming in a subject, the method comprising: (a) obtaining a biological sample derived from the subject before and after the treatment; (b) detecting an amount and/or an activity of a plurality of biomarkers from the biological sample, wherein the plurality of biomarkers comprise a transposable element transcript transcribed from a transposable element, wherein the transposable element comprises a sequence that is at least 60%, at least 70%, at least 80%, at least 90%, or at least 95% identical to at least a portion of a sequence selected from of SEQ ID NOs: 4, 7-11, and 14-121; and (c) concluding the treatment is effective if the amount and/or the activity after the treatment is at least 50%, at least 60%, at least 70%, at least 80%, at least 90% lower when compared to the amount and/or the activity before the treatment. In some instances, the sample is an alveolar type 2 cell (AT2 cell) or a lung fibroblast. In some instances, if the pulmonary reprogramming is not sufficient, the methods further comprise d) administering to the subject the modulator described herein or the pharmaceutical composition described herein. In some instances, the modulator described herein or the pharmaceutical composition described herein is administered intratracheally, oropharyngeally, orally, nasally, intravenously, intraperitoneally, or intramuscularly. In some instances, the plurality of biomarkers comprise one or more fibrosis-related markers (e.g., smooth muscle α actin (ACTA2), alpha 1 chain of collagen type I (COL1A1), alpha 1 chain of collagen type 3 (COL3A1), fibroblast activation protein (FAP), fibronectin 1 (FN1), periostin (POSTN), collagen triple helix repeat containing 1 (CTHRC1), alpha 1 chain of collagen type 10 (COL10A1), matrix metalloproteinase 11 (MMP11), thrombospondin 2 (THBS2), latent transforming growth factor beta binding protein 2 (LTBP2), Integrin-α11 (ITGA11), TERT, or a combination thereof, or one or more genes identified in Table 3 and Table 5). In some instances, the detecting comprises using S1 nuclease protection assay, microarray analysis, polymerase chain reaction (PCR), hybridization technologies, reverse transcriptase polymerase chain reaction (RT-PCR), Northern blot, serial analysis of gene expression (SAGE), immunoassay, and/or mass spectrometry, or any sequencing-based methods known in the art. In some instances, exemplary any sequencing-based methods include but are not limited to bulk RNA sequencing, single cell or single nucleus RNA sequencing, DNA sequencing, DNA methylation profiling (e.g., for example, average % methylated CpG along the transposable element locus or the methylation status of specific CpGs within the transposable element), sc- or snATAC-seq or bulk ATAC-seq, DNase I assay for accessibility, CUT&RUN or ChIP-seq for specific histone marks (e.g., H3K27ac, H4K16ac, H3K9me3, H3K27me3, H3K4me1, H3K4me3, etc.).

### Other Terminology

The term “noncoding RNA” as used herein, refers to RNA species that are not translated into proteins. The term “long noncoding RNA” or “lncRNA” as used herein, refers to a noncoding RNA that is at least 100, 200, 300, 400, 500, 600, 700, 800, 900, or 1000 nucleotides long.

The term “healthy cell” as used herein refers to in healthy cells of a healthy individual. In some instances, it refers to a cell of the same subject but before contracting any disorders, such as pulmonary fibrosis.

The term “nucleic acid analogue,” as used herein, can refer to compounds which are analogous (structurally similar) to naturally occurring nucleic acid (see, e.g., Freier & Altmann; Nucl. Acid. Res., 1997, 25, 4429-4443 and Uhlmann; Curr. Opinion in Drug Development, 2000, 3 (2), 293-213), and examples of suitable nucleic acid analogues are provided by WO2007031091, which are hereby incorporated by reference.

The term “Gapmer” is a chimeric nucleic acid that contains a central sequence of phosphorothioate DNA nucleotides (“DNA gap”) flanked by sequences of modified RNA residues at either end to protect the DNA gap from nuclease degradation, whereas the central DNA gap region allows RNase-H-mediated cleavage of the target RNA. Gapmer has an internal region having a plurality of nucleosides which is capable of recruiting RNase H activity, such as RNaseH, which region is positioned between external wings at either end, having one or more nucleosides, wherein the nucleosides comprising the internal region are chemically distinct from the nucleoside or nucleosides comprising the external wings.

A “locked nucleic acid” or “LNA” is often referred to as inaccessible RNA, and is a modified RNA nucleobase. The ribose moiety of an LNA nucleobase is modified with an extra bridge connecting the 2′ oxygen and 4′ carbon. An LNA oligonucleotide offers substantially increased affinity for its complementary strand, compared to traditional DNA or RNA oligonucleotides.

The terms “microRNA,” “miRNA,” and MiR” are interchangeable and refer to endogenous or artificial non-coding RNAs that are capable of regulating gene expression. It is believed that miRNAs function via RNA interference. The terms “siRNA” and “short interfering RNA” are interchangeable and refer to single-stranded or double-stranded RNA molecules that are capable of inducing RNA interference. In some aspects, siRNA molecules typically have a duplex region that is between 18 and 30 base pairs in length.

The terms “piRNA” and “Piwi-interacting RNA” are interchangeable and refer to a class of small RNAs involved in gene silencing. piRNA molecules typically are between 26 and 31 nucleotides in length.

The terms “snRNA” and “small nuclear RNA” are interchangeable and refer to a class of small RNAs involved in a variety of processes including RNA splicing and regulation of transcription factors. The subclass of small nucleolar RNAs (snoRNAs) is also included. The term is also intended to include artificial snRNAs, such as antisense derivatives of snRNAs comprising antisense sequences directed against one or more transposable element.

The term “LTR16” used herein is to refer to a specific subfamily of transposable elements which share a consensus sequence set forth in SEQ ID NO: 10.

The terms “polynucleic acid,” “polynucleotide,” “oligonucleotide,” “nucleic acid” and “nucleic acid molecule” are used herein to include a polymeric form of nucleotides of any length, either ribonucleotides or deoxyribonucleotides. This term refers to the primary structure of the molecule. Thus, the term includes triple-, double- and single-stranded DNA, as well as triple-, double- and single-stranded RNA. In some aspects, it also includes modifications, such as by methylation and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), any other type of polynucleotide which is an N- or C-glycoside of a purine or pyrimidine base, and other polymers containing nonnucleotidic backbones, for example, polyamide (e.g., peptide nucleic acids (PNAs)) and polymorpholino (commercially available from the Anti-Vials, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA. There is no intended distinction in length between the terms “polynucleotide,” “oligonucleotide,” “nucleic acid” and “nucleic acid molecule,” and these terms will be used interchangeably. Thus, these terms include, for example, RNA, double- and single-stranded DNA, as well as double- and single-stranded RNA, microRNA, DNA:RNA hybrids, and hybrids between PNAs and DNA or RNA, and also include known types of modifications, for example, labels which are known in the art, methylation, “caps,” substitution of one or more of the naturally occurring nucleotides with an analog (e.g., 2′-aminoadenosine, 2′-thiothymidine, inosine, pyrrolo-pyrimidine, 3′-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2′-thiocytidine), internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide. The term also includes locked nucleic acids (e.g., comprising a ribonucleotide that has a methylene bridge between the 2′-oxygen atom and the 4′-carbon atom). See, for example, Kurreck et al. (2002) Nucleic Acids Res. 30:1911-1918.

The term “complementary” and “complementarity” are interchangeable and refer to the ability of polynucleotides to form base pairs with one another. Base pairs are typically formed by hydrogen bonds between nucleotide units in antiparallel polynucleotide strands or regions. Complementary polynucleotide strands or regions can base pair in the Watson-Crick manner (e.g., A to T, A to U, C to G). 100% complementary refers to the situation in which each nucleotide unit of one polynucleotide strand or region can hydrogen bond with each nucleotide unit of a second polynucleotide strand or region. Less than perfect complementarity refers to the situation in which some, but not all, nucleotide units of two strands or two regions can hydrogen bond with each other and can be expressed as a percentage.

“Administering”, as it applies in the present disclosure, refers to contact of an effective amount of a modulator of a transposable element of the disclosure or a synthetic polynucleic acid comprising a nucleic acid sequence of a transposable element or a portion thereof to the subject. Administering a nucleic acid, such as a microRNA, siRNA, piRNA, snRNA, or antisense nucleic acid, to a cell comprises transducing, transfecting, electroporating, translocating, fusing, phagocytosing, shooting or ballistic methods, or any means by which a nucleic acid can be transported across a cell membrane.

“Pharmaceutically acceptable excipient or carrier” refers to an excipient that may optionally be included in the compositions of the disclosure and that causes no significant adverse toxicological effects to the patient.

“Pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly, salts containing pharmaceutically acceptable cations include, but are not limited to, sodium, potassium, calcium, aluminum, lithium, and ammonium (including substituted ammonium).

An “effective amount” of modulator of transposable element of the disclosure (e.g., microRNA, siRNA, piRNA, snRNA, antisense nucleic acid, ribozyme, or small molecule inhibitor, CRISPRs etc.) or a synthetic polynucleic acid is an amount sufficient to effect beneficial or desired results, such as an amount that inhibits, activates, or supplement the activity of the transposable element. In some instances, the effective amount of the modulator is enough to interfere with the transcription of the transposable element. An effective amount can be administered in one or more administrations, applications, or dosages. The exact amount required will vary from subject to subject, depending on the species, age, and general condition of the subject, the severity of the condition being treated, the particular drug or drugs employed, mode of administration, and the like. An appropriate “effective” amount in any individual case may be determined by one of ordinary skill in the art using routine experimentation, based upon the information provided herein.

“Homology” refers to the percent identity between two polynucleotide or two polypeptide moieties. Two nucleic acid sequences, or two polypeptide sequences are “substantially homologous” to each other when the sequences exhibit at least about 50% sequence identity, at least about 75% sequence identity, at least about 80%-85% sequence identity, at least about 90% sequence identity, or about 95%-98% sequence identity over a defined length of the molecules. As used herein, substantially homologous sequences also refer to sequences showing complete identity to the specified sequence.

In general, “identity” refers to an exact nucleotide to nucleotide or amino acid to amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100. Alternatively, homology can be determined by readily available computer programs or by hybridization of polynucleotides under conditions which form stable duplexes between homologous regions, followed by digestion with single stranded specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art.

As used herein, a “sample” refers to a sample of tissue or fluid isolated from a subject, including but not limited to, for example, urine, blood, plasma, serum, fecal matter, bone marrow, bile, spinal fluid, lymph fluid, samples of the skin, external secretions of the skin, respiratory, intestinal, and genitourinary tracts, tears, saliva, milk, blood cells, organs, biopsies, and also samples containing cells or tissues derived from the subject and grown in culture, and in vitro cell culture constituents, including but not limited to, conditioned media resulting from the growth of cells and tissues in culture, recombinant cells, stem cells, and cell components.

The terms “quantity,” “amount,” and “level” are used interchangeably herein and may refer to an absolute quantification of a molecule or an analyte in a sample, or to a relative quantification of a molecule or analyte in a sample, i.e., relative to another value such as relative to a reference value as taught herein, or to a range of values for the biomarker. These values or ranges can be obtained from a single patient or from a group of patients.

Whenever the term “at least,” “more than,” or “less than” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

The term “a,” “an” and “the” include plural referents unless the content clearly dictates otherwise. Thus, for example, reference to “a transposable element” includes a mixture of two or more transposable elements, and the like.

The term “pulmonary fibrosis” as used herein, refers to a set of lung diseases that affect the respiratory system. In some instances, pulmonary fibrosis refers to thickening or scarring of the lung tissue or a portion thereof. In some instances, pulmonary fibrosis is idiopathic pulmonary fibrosis.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

The term “about,” as used herein, when referring to a number or a numerical range, generally means that the number or numerical range referred to is an approximation within experimental variability (or within statistical experimental error), and thus, the number or numerical range, in some instances, may vary from the stated number or numerical range.

The term “comprising” (and related terms such as “comprise” or “comprises” or “having” or “including”) is not intended to exclude that in other certain instances, for example, an instance of any composition of matter, composition, method, or process, or the like, described herein, “consist of” or “consist essentially of” the described features.

The term “subject” or “patient,” as used herein, generally encompasses organisms such as mammals. Examples of mammals include, but are not limited to, any member of the mammalian class: humans, non-human primates, such as chimpanzees, and other apes and monkey species; farm animals, such as cattle, horses, sheep, goats, swine; domestic animals, such as rabbits, dogs, and cats; laboratory animals, including rodents, such as rats, mice and guinea pigs, and the like. In one aspect, the mammal is a human.

The term “treatment” or “treating,” as used herein, are used interchangeably. These terms generally refer to an approach for obtaining beneficial or desired results including but not limited to therapeutic benefit and/or a prophylactic benefit. In some instances, the term refers to eradication of the underlying disorder being treated. In other instances, the term refers to the eradication of one or more of the physiological symptoms associated with the underlying disorder such that an improvement is observed in the patient, notwithstanding that the patient is still afflicted with the underlying disorder.

The term “alleviating” or “alleviate,” as used herein, refers to amelioration, improving, or stalling the further progression of the underlying disorder being treated. In other instances, the term refers to the amelioration, improving, or stalling the further progression of one or more of the physiological symptoms associated with the underlying disorder such that an improvement is observed in the patient, notwithstanding that the patient is still afflicted with the underlying disorder.

The term “preventing” or “prevent,” as used herein, refers to the situation where the compositions disclosed herein are administered to a patient at risk of developing a particular disease, or to a patient reporting one or more of the physiological symptoms of a disease, even though a diagnosis of this disease has not been made.

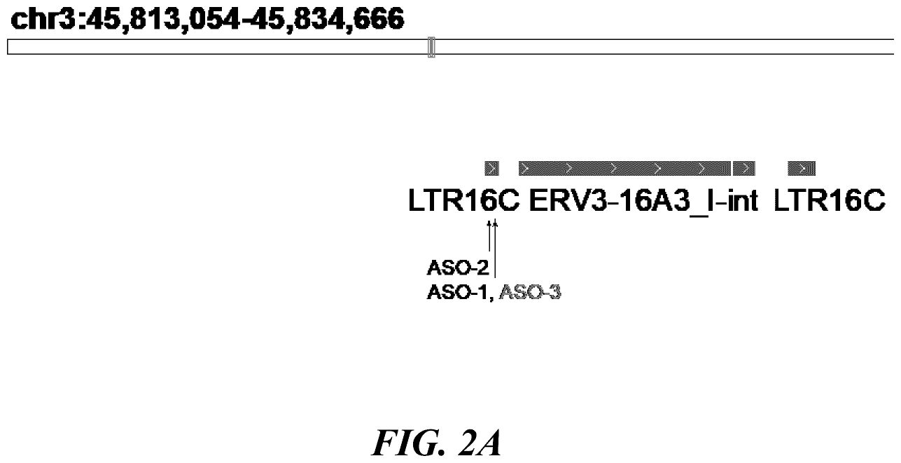

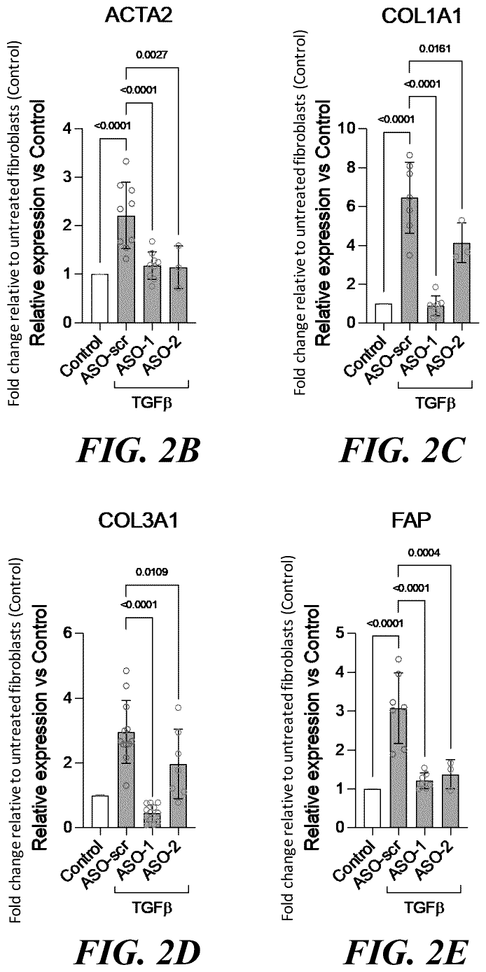

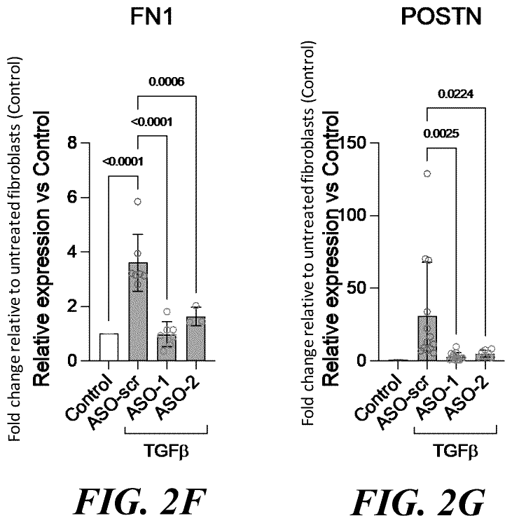

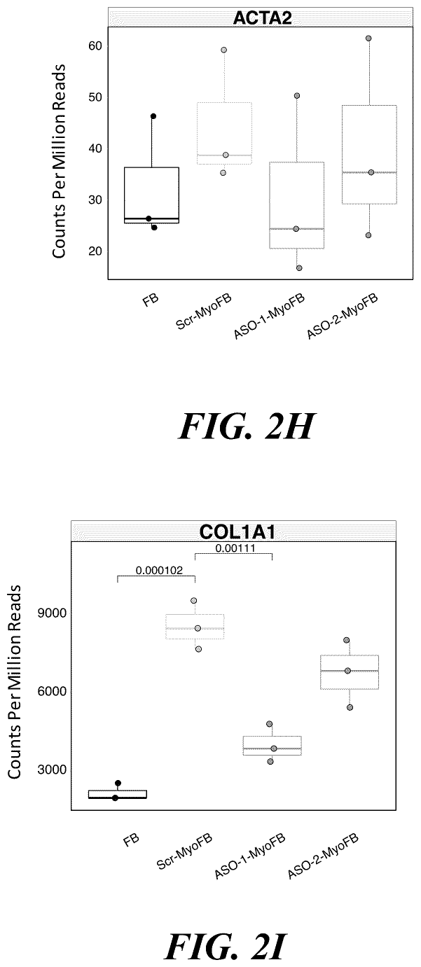

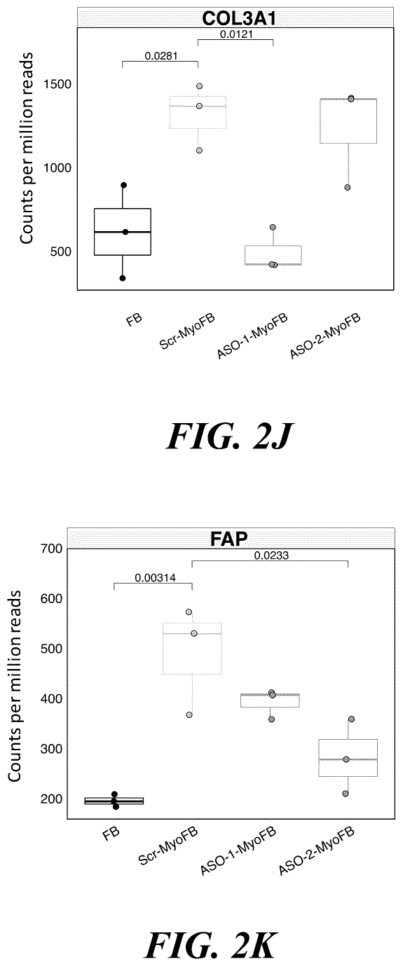

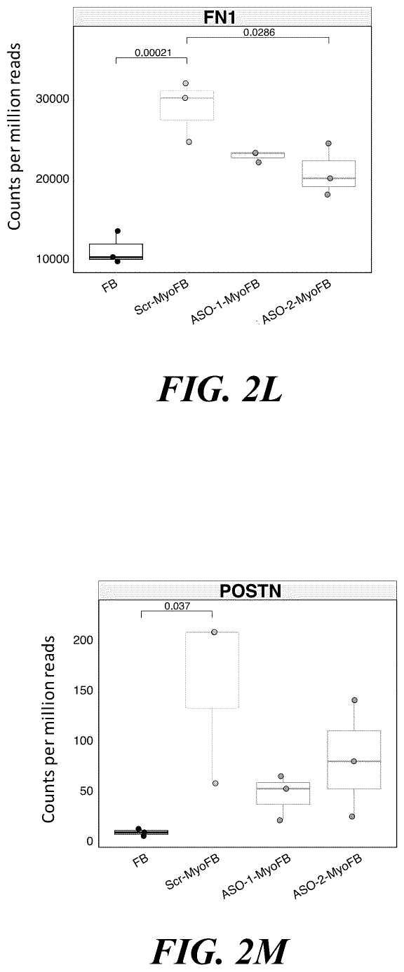

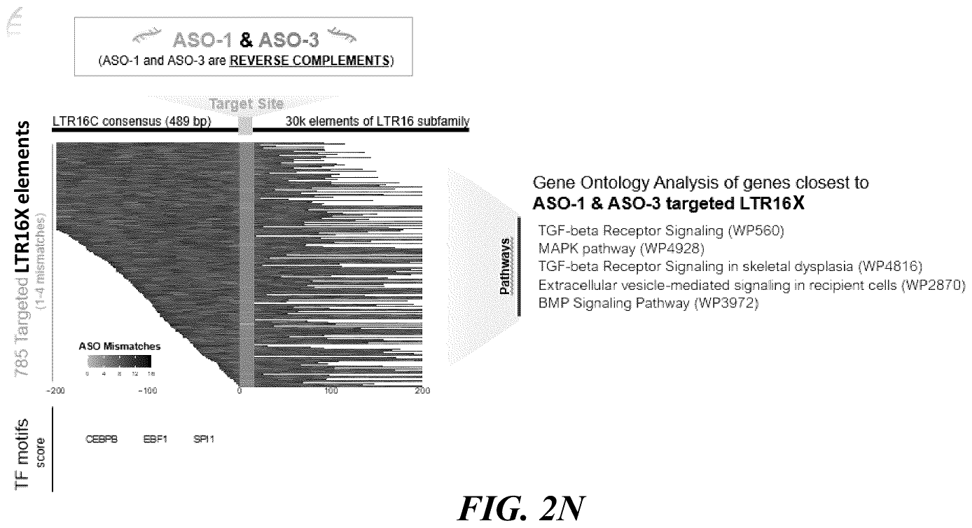

The term “LTR16X” as used herein is an umbrella term that includes subfamilies that ASO-1 or ASO-3 targets as shown in FIG. 2N and Table 4. The targeted subfamilies include LTR16, LTR16A, LTR16A1, LTR16A2, LTR16B, LTR16B1, LTR16B2, LTR16C, LTR16D, LTR16D1, LTR16D2, LTR16E1, LTR16E2, HERV16, ERV3-16A3_LTR, and ERV3-16A3_I subfamilies. The consensus sequences of these subfamilies can be found in Table 1. “HERV16-int” and “HERV16” are used herein interchangeably to refer to a subfamily with a full-length internal provirus region, and respectively “ERV3-16A3_I-int” and “ERV3-16A3_I” are used herein interchangeably to refer to a subfamily with a full-length internal provirus region.

The term “LTR16X ASO-1” or an equivalent expression is used herein interchangeably with “ASO-1” and refers to a base sequence of SEQ ID NO: 1 with a 3-11-2 LNA GapmeR configuration and a phosphorothioate linkage between every nucleotide. The term “LTR16X ASO-2” or an equivalent expression is used herein interchangeably with “ASO-2” and refers to a base sequence of SEQ ID NO: 2 with a 3-11-2 LNA GapmeR configuration and a phosphorothioate linkage between every nucleotide. The term “LTR16X ASO-3” is used herein interchangeably with “ASO-3” and refers to a base sequence of SEQ ID NO: 3 with a 3-11-2 LNA GapmeR configuration and a phosphorothioate linkage between every nucleotide. When stating that ASO-1, ASO-2, or ASO-3 targets LTR16C, it should not be interpreted as LTR16C is the only target of ASO-1, ASO-2, or ASO-3.

The term “n1-n2-n3 configuration” used herein refers to a structure of a Gapmer, wherein n1 refers to the number of nucleic acid analogues as a 5′-wing region, n2 refers to the number of consecutive DNA nucleotides in the central region, and n3 refers to the number of nucleic acid analogues as a 3′-wing region.

The term “integrant” or “locus” as used herein refers to a single transposable element locus with specific coordinates. Therefore, as used herein, a transposable element superfamily comprises numerous integrants from different genomic locations. In other words, an integrant is a single locus and a subfamily is a group of similar integrants.

The term “extracellular-cellular-matrix synthesizing state” as used herein, in some instances, refers to a cell state where the cell has higher expression of one or more fibrotic markers (e.g., ACTA2, COL1A1, COL3A1, FAP, FN1, or POSTN). The term “cell pausing state” or “quiescence” as used herein, in some instances, refers to a cell state where the cell stays in G1 stage in a cell cycle. The term “cell dividing state” as used herein, in some instances, refers to a cell state where the cell stays in G2/M/S stage in a cell cycle.

The term “transposable element transcript” as used herein refers to a transcript transcribed from a transposable element DNA locus.

The term “consensus sequence” as used herein refers to an averaging modeling to group similar integrants that share certain degree of sequence homology.

The term “reprogram” “reprogramming” or “cellular reprogramming” are used interchangeably to refer to a process that alters or reverses a differentiation state of a differentiated cell (e.g., myofibroblast). In some instances, reprogramming can be a complete or full reversal of the differentiation state to a pluripotent state or a multipotent state. In some instances, reprogramming can be a complete or partial reversion of the differentiation state of a differentiated cell (e.g., myofibroblast) to an undifferentiated cell, or a less differentiated state.

While various instances of the disclosure have been shown and described herein, it will be obvious to those skilled in the art that such instances are provided by way of example only. Numerous variations, changes, and substitutions may occur to those skilled in the art without departing from the disclosure. It should be understood that various alternatives to the instances of the disclosure described herein may be employed.

### EXAMPLES

The following is a description of various non-limiting examples of methods and materials used in the studies, and are put forth so as to provide those of ordinary skill in the art with a complete disclosure and description of how to make and use the present disclosure, and are not intended to limit the scope of the disclosure nor are they intended to represent that the experiments below were performed and are all of the experiments that may be performed. Efforts have been made to ensure accuracy with respect to numbers used (e.g., amounts, percentages, etc.), but some experimental errors and deviations should be accounted for.

### Example 1: Characterization of TEs that are Associated with Pulmonary Fibrosis

Transposable elements (TE) are major components of eukaryotic genomes that can be involved in various functions. In this Example, TEs that are expressed in human lung fibroblasts and human lung myofibroblasts were identified.

As an overall strategy, targets were identified by prioritizing sub-families differentially expressed across different data modalities, including bulk as well as single cell or single nuclei expression datasets from lung biopsies or in vitro experiments. Sub-families were ordered according to their rank product across datasets (Breitling et al., 2004), with high-ranking sub-families corresponding to those consistently upregulated in myofibroblasts (in vitro or from diseased patients). This approach resulted in 27 candidate sub-families. To narrow down our selection of sub-families relevant for idiopathic pulmonary fibrosis, individual integrants for each of the 27 candidates overlapping predicted regulatory elements (enhancers) in a lung cell line (IMR90) as well as in vitro primary fibroblasts and myofibroblasts were identified. Finally, for each sub-family, the fraction of integrants in enhancers bearing binding sites for two transcription factors (SPI1 and EBF1) which have clinical relevance for IPF (Wohlfahrt et al., 2019; Liu et al., 2021) were determined.

Specifically, deep RNA-seq at 200 million reads from human lung fibroblasts and human lung myofibroblasts was performed, and TEs were mapped to the reference genome with a curated version of the Repeatmasker database added to it. For bulk RNASeq datasets, transposable elements expression was quantified with SalmonTE (v0.4, Jeong et al., 2018) and internally customized database of TE elements. SalmonTE is a wrapper around the tool Salmon (v0.8.2, Patro et al., 2017) to quantify transcripts expression. Raw reads were trimmed with Trimmomatic (v0.3.6, Bolger et al., 2014). Quantification was carried out with Salmon quant (via SalmonTE) on the trimmed reads against the index built for sub-families or loci-level. Sub-families with less than ten transcripts assigned in at least half of the samples were filtered out. Finally, differentially expressed sub-families in R (v4.3.2, R Core Team, 2023) were identified with the DESeq2 Bioconductor package (v1.42.0, Love et al., 2014). For database building, to quantify expression of sub-families, the general database described above was formatted to create an index against which Salmon (via SalmonTE) would align and quantify TE transcripts. To achieve locus-level quantification, the general database was modified by assigning a unique identifier to each locus. To build a general transposable element database, a transposable element database was built by adapting the RepeatMasker table from UCSC (hg38, downloaded on Apr. 25, 2022). The table was filtered to keep only SINE, LINE, LTR, DNA and RC repeats.

For experiments herein, primary NHLFs (normal human lung fibroblast) from patient biopsies (Lonza, ref. CC-2512) were cultured with fibroblast growth medium-2 (FGM-2). NHLFs were transfected with antisense oligonucleotides (ASOs) described herein for 24 hours, and medium was changed to serum-free medium containing 5 ng/ml TGFβ for differentiation to myofibroblasts for 24 hours. Nuclei were extracted according to protocol CG0000124 Rev F from 10× Genomics. snRNA-seq was performed with Chromium X according to protocol CG000204 Rev D from 10X Genomics.

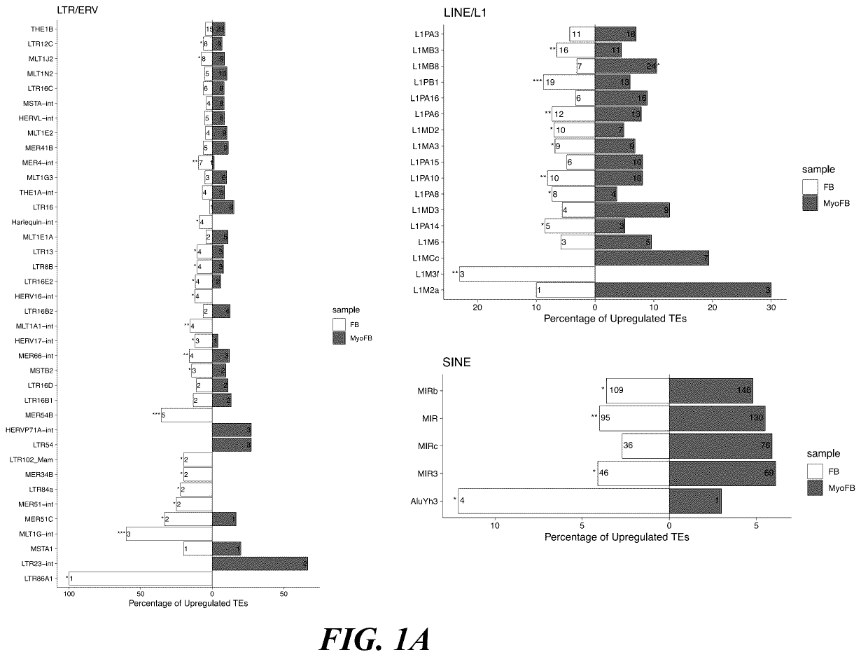

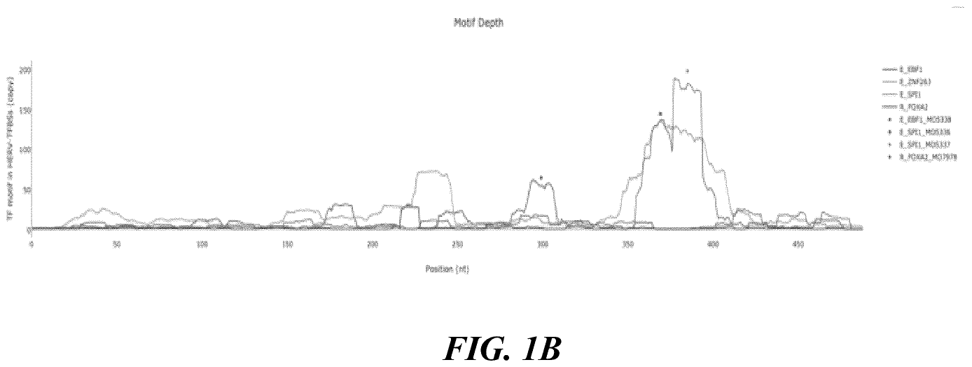

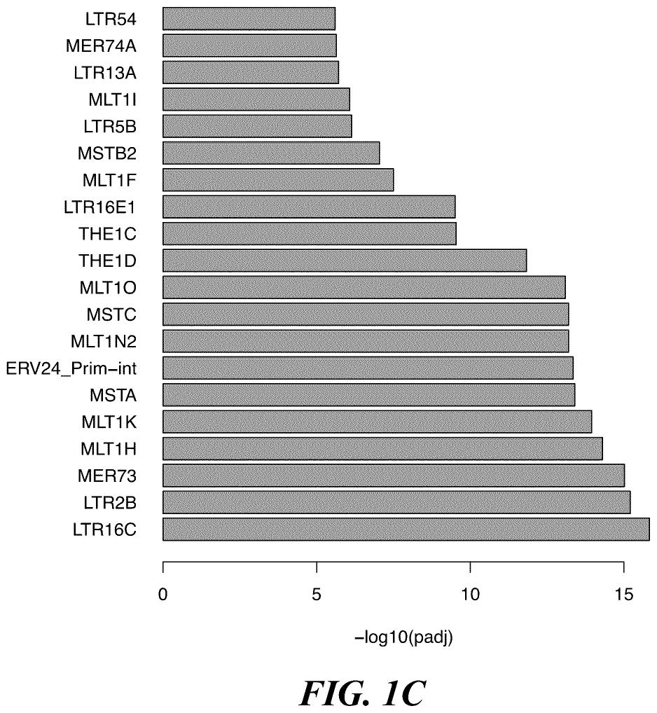

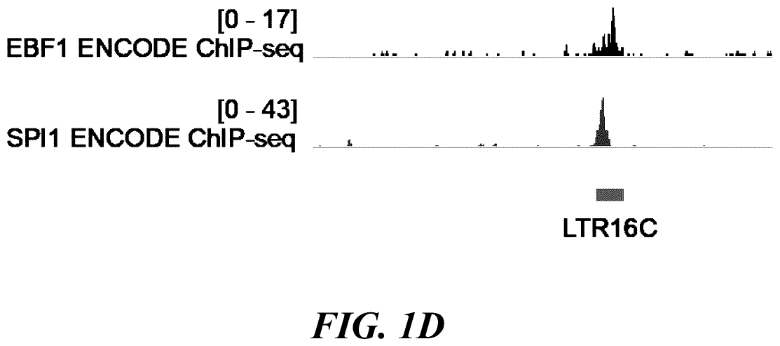

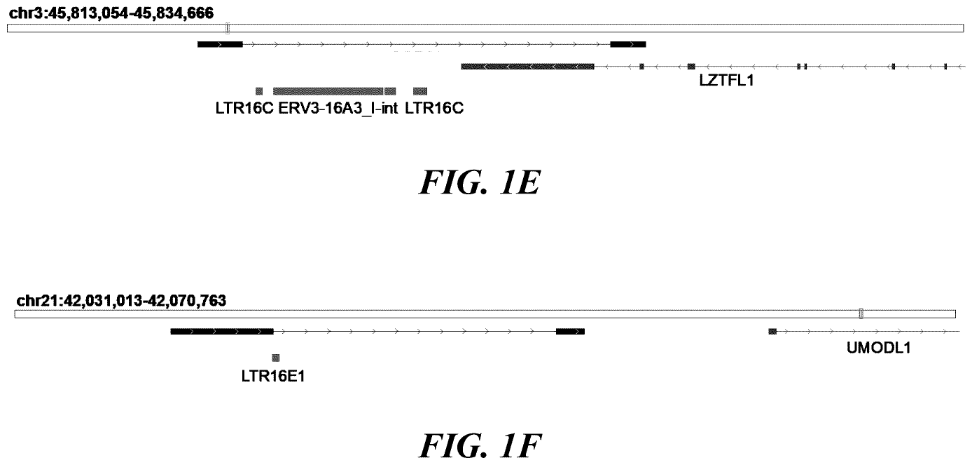

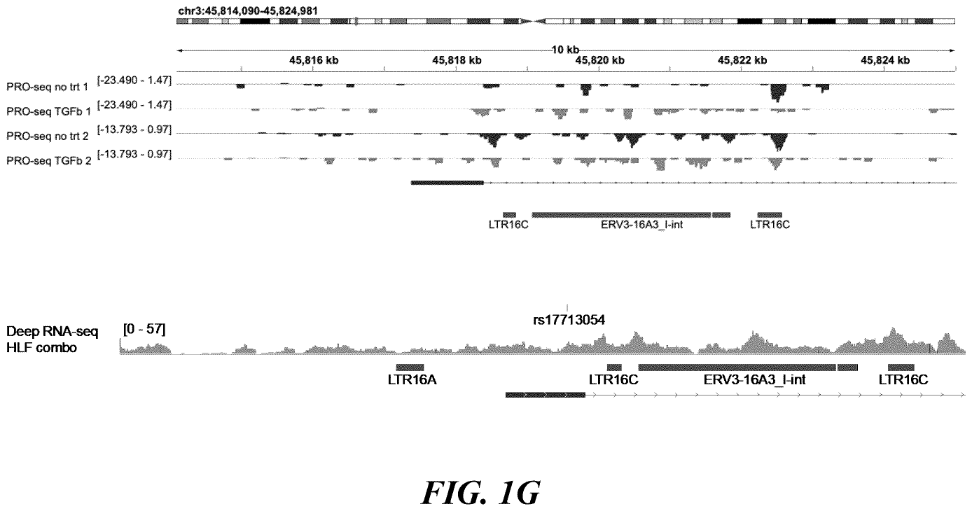

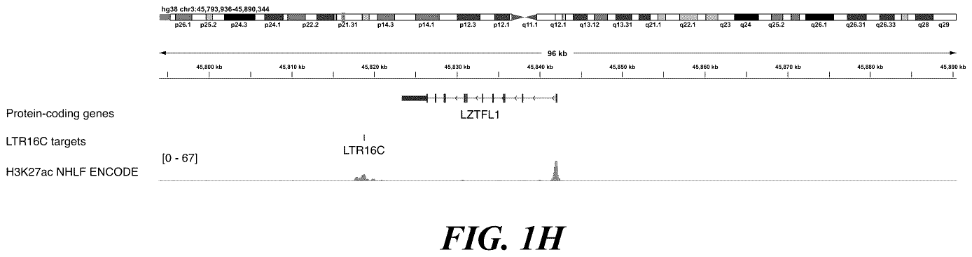

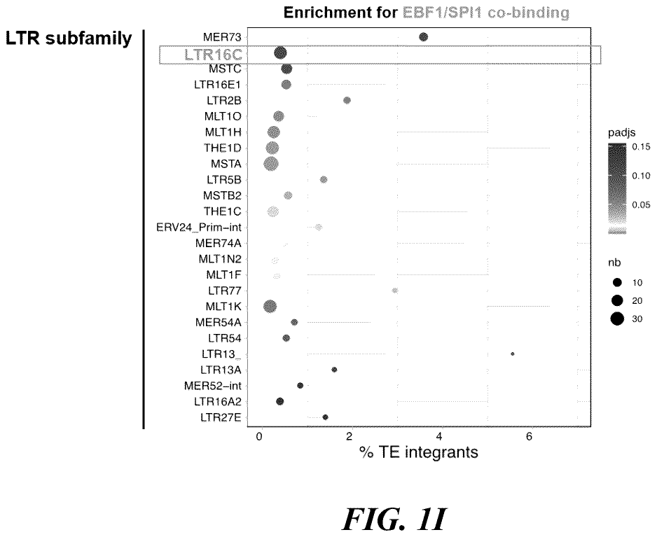

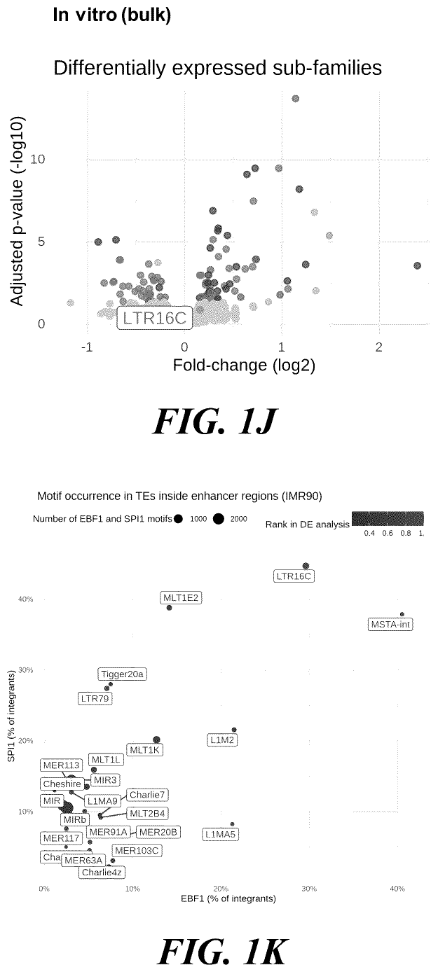

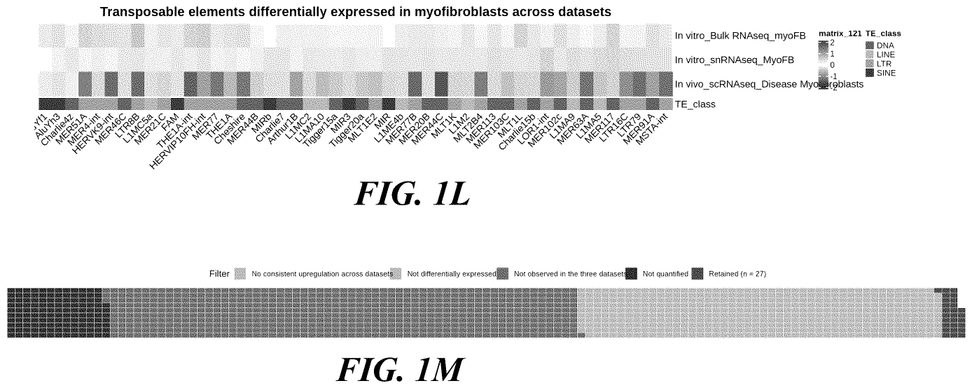

Differential expression of various superfamilies/families/subfamilies of TEs were examined. The various superfamilies/families/subfamilies of TEs include long terminal repeat retrotransposons (LTR) (e.g., endogenous retrovirus (ERV)) and non-LTR retrotransposons, for example, long and short interspersed nuclear elements (LINEs and SINEs). As shown in FIG. 1A, a subset of members of TE subfamily in a TE family showed downregulation in human lung myofibroblasts while another subset of members in the same TE subfamily showing showed upregulation in human lung myofibroblasts. For example, in the subfamily of THE1B, 15 TEs were downregulated in MyoFB and 28 TEs were upregulated. Consequently, the 28 TEs that were upregulated were identified as potential targets for inhibition and were subject to further evaluation. The 15 TEs can also be evaluated with overexpression methods. For example, the mRNA of one or more of the 15 FB-specific ones are encapsulated in an LNP, and delivered to myofibroblasts. As a result, the modified myofibroblasts is converted to a more fibroblast-like state.

Since LTRs are frequently coopted as regulatory elements and display evolutionary convergence between species between similar but not identical subfamilies, LTRs were further evaluated. To evaluate the functional aspects of the identified LTRs in lung tissues and/or lung cells (e.g., pulmonary fibroblast, etc.), the TEs were analyzed using an interactive database of human ERV/LTR regulatory elements for possible enrichment of EBF1 and SPI binding motifs, which are associated with lung myofibroblast polarization and idiopathic pulmonary fibrosis (IPF) population, respectively. As shown in FIG. 1B, enriched motifs for EBF1 and SPI transcription factor binding sites were identified across the consensus sequence of the LTR16C elements in the HERV-transcription factor-binding site (TFBS) public database (see, e.g., Ito, Sugimoto, Nakaoka, Yamada, Kimura, Hayano, et al.; PLOS Genet 2017, 13 (7): e1006883). X-axis in the graph indicates LTR16C consensus position and γ-axis indicates number of copies of the motif that were identified at each consensus position. The line shows ChIP signal for select transcription factors (e.g., EBF1, SPI1) overlaid on the LTR16C consensus sequence, while the dots mark the locations where the motif for the corresponding transcription factor is found. To validate this finding and to identify which LTRs are co-bound by EBF1 and SPI1, public ENCODE ChIP-seq datasets for EBF1 and SPI1 transcription factors were profiled for genome-wide co-binding events on LTRs. Briefly, the binding sites for EBF1 and SPI1 datasets were obtained for the Genome Reference Consortium Human Build 38 (GRCh38/hg38) from ENCODE (e.g., in bed file format usable with thebedtools program). Then, the transposable element datasets, including one sub-selecting LTR families only, were intersected with SPI1 with bedtools, keeping the LTRs bound by SPI1. The resulting data was intersected with EBF1 with bedtools to generate a list of LTRs bound by both EBF1 and SPI1, and enrichment analysis (e.g., binomial test) was performed. As shown in FIG. 1C, FIG. 1I, and FIG. 1K, LTR16C was more enriched with EBF1 and SPI1 binding sites compared to other LTRs. FIG. 1D further verifies enrichment for both EBF1 and SPI binding motifs at the LTR16C genomic location, suggesting LTR16C may be involved in the regulation of lung myofibroblast polarization and IPF.

To further identify the regulatory function of LTR16C, the genomic location and expression of LTR16C in the human lung fibroblast and human lung fibroblast treated with serum-free medium containing TGFβ (myofibroblast) were evaluated. As shown in FIG. 1E, the genomic location of LTR16C was probed for in the human genome in chromosome 3:45,818,666-45,818,847. LTR16E1, a closely related family of LTR16C, was found to be located in chromosome 21:42,0419,09-42,042,242, as shown in FIG. 1F. Next, the expression of LTR16C was analyzed by PRO-seq of human lung fibroblast (HLF) with or without TGFβ. The expression of LTR16C was also analyzed by deep RNA-seq data of human lung fibroblasts with TGFβ plus serum starvation (“HLF combo”). For HLF combo, the medium of the human lung fibroblasts was changed to serum-free medium containing 5 ng/ml TGFβ. As shown in FIG. 1G, LTR16C reads were detected by PRO-seq and deep RNA-seq data, confirming the enriched expression of LTR16C in human lung fibroblasts with TGFβ.

Single-cell and single-nuclei-based TE quantification was performed as follows. For single cell and single nuclei datasets, TE sub-families were quantified along with other features (e.g., protein coding genes, lncRNAs, etc.) with scTE (v1.0, He et al., 2021). Raw reads were trimmed and mapped with 10× Genomics Cell Ranger (v7.1.0) to produce BAM files. Mapped reads with known-good cellular barcodes were kept with samtools v1.17 (samtools view-bhd CB, Danecek et al., 2021).

An index was built with the scTE_build command including in-house GTF and the general transposable elements database described above. Quantification was carried out with scTE with default parameters using this index and the filtered BAM files as inputs. QC filtering and differential expression analysis were performed in R (v4.3.2, R Core Team, 2023) with the Seurat package (v5.0.1, Hao et al., 2023). Features (i.e., TE sub-families, protein-coding genes, lncRNAs, etc.) that were on the top 15th percentile of the distribution of assigned reads in each sample were retained. The resulting cells were then clustered and visualized in two dimensions with a UMAP reduction technique with the FindClusters and RunUMAP commands, respectively.

When comparing in vitro experiments, the expression of marker genes and cluster composition were leveraged to re-assign labels to cells that originated from fibroblast cell cultures but had a transcriptomic profile closer to myofibroblasts and vice versa. Differential expression of features were tested on the newly labelled cells with the function FindMarkers. On the other hand, when analyzing published datasets, cell labels as published in the original study and identified markers upregulated in cells from myofibroblasts of IPF patients were compared against other mesenchymal cell types of IPF and healthy donors (including myofibroblasts) with the function FindAllMarkers.

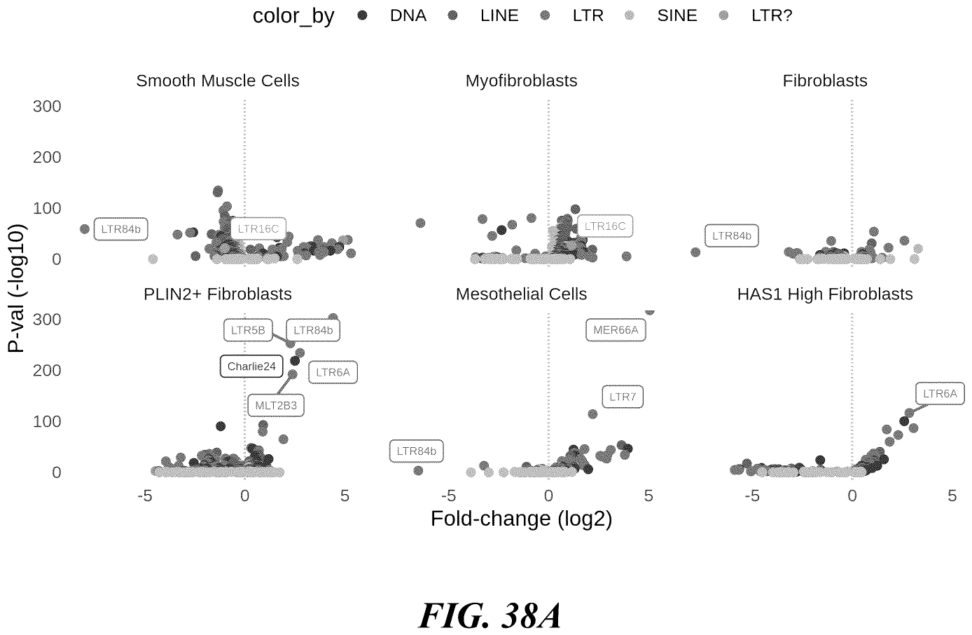

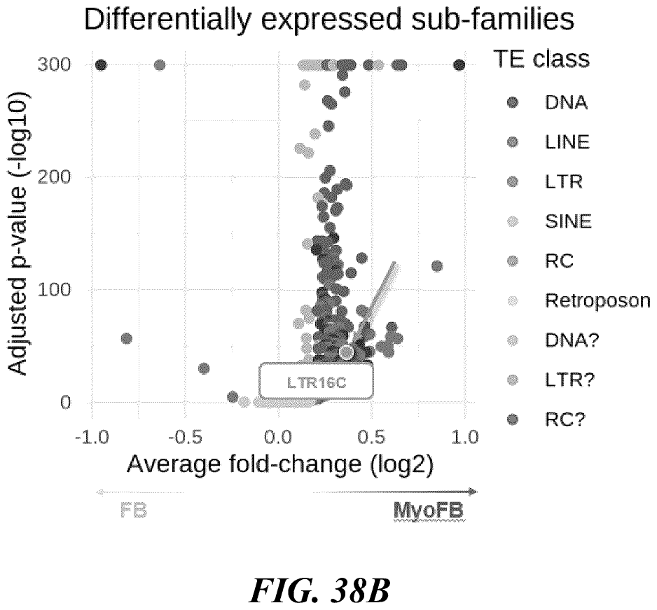

LTR16C was also found to be significantly upregulated in myofibroblasts compared to other mesenchymal cell types, as observed by analyzing the publicly available idiopathic pulmonary fibrosis (IPF) cell atlas (FIG. 38A, FIG. 1L). Similarly, as shown in FIG. 38B, FIG. 1J, and FIG. 1L, single nucleus RNA-sequencing (snRNA-seq) and bulk RNA-sequencing analysis of the in vitro human lung fibroblasts cultured in serum-free medium containing 5 ng/ml TGFβ, confirmed that LTR16C subfamily is part of the myofibroblast signature. FIG. 1M shows subfamily-level filters that were excluded from the respective snRNA-seq and bulk RNA-seq data. In addition, ChromHMM analysis, which is a computational approach to annotate chromatin states in one or multiple cell types, was performed on Roadmap data for the lung fibroblast cell line (IMR90).

Specifically, ChromHMM was carried out as follows. Regulatory elements were predicted along the human genome (hg38 build) with ChromHMM v1.24 (Ernst et al., 2012) and a set of seven epigenetic marks (H3K27ac, H3K27me3, H3K36me3, H3K4me1, H3K4me2, H3K4me3, H3K9me3) profiled with ChIP-Seq on the fetal lung cell line IMR90 (Schultz et al., 2015). Transposable element loci from 27 candidate subfamilies were then checked for enhancer status. For those identified as enhancers, the sequences were scanned for transcription factor binding sites motifs with fimo (v5.0.5, Grant et al., 2011) and the CIS-BP motifs database (v2.00, Weirauch et al., 2014)

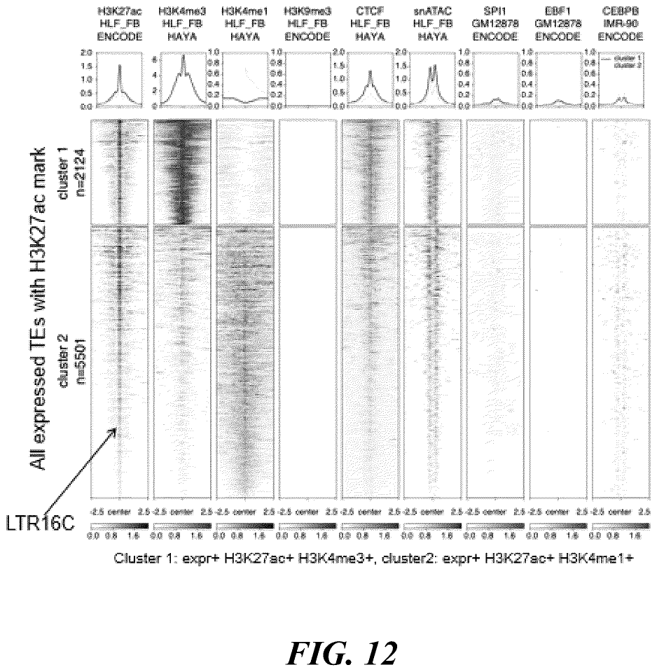

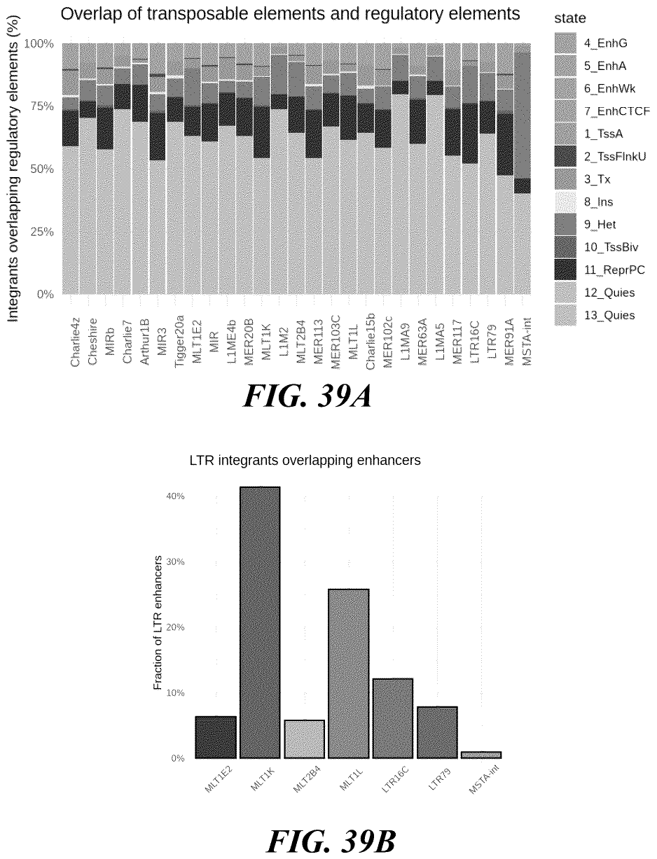

As shown in FIG. 39A, LTR16C elements were among regulatory elements contributed by transposable elements for this lung fibroblast cell line. LTR16C was also found to be overlapping with enhancers, as shown in FIG. 39B. The ChromHMM results shown in FIG. 39A were filtered to show only the subfamilies selected on the basis of expression depicted as “retained” in FIG. 1M. With a further filter-only LTRs from this subset, FIG. 39B shows which percentage of the total loci had enhancer marks. Furthermore, public human lung fibroblast ENCODE data for H3K27ac was analyzed at the LTR16C locus as another way to confirm activity of LTR16C in human lung fibroblast since enrichment of the epigenetic marker, H3K27ac, is associated with gene activation. As shown in FIG. 1H, H3K27ac marks were observed in LTR16C region. In addition, expressed TEs with H3K27ac from ENCODE were subselected as shown in FIG. 12. Signal for various Cut&Run and ChIP datasets from ENCODE and in-house dataset were overlaid. The analysis split them further into 2 groups: H3k4me3+ and H3K4me1+. The H3K4me1+ can be the enhancer class and can list interesting drug targets.

### Example 2: Effect of LTR16X Antisense Oligonucleotides (ASO) on Fibrosis Marker Expression

To investigate the effect of inhibition of transcripts encoded by LTR16C, two antisense oligonucleotides (ASO-1 and ASO-2) were designed to target a transcript encoded by LTR16C, which is mapped to chr3: 45,818,736-45,818,762 and chr3: 45,818,689-45,818,712, respectively, as shown in FIG. 2A. ASO-1 and ASO-2 are ASOs with a 3-11-2 LNA GapmeR configuration and a phosphorothioate linkage was introduced between every nucleotide of ASO-1 and ASO-2.

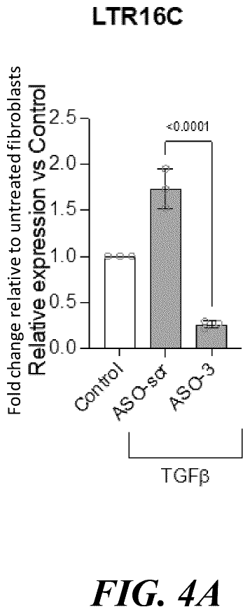

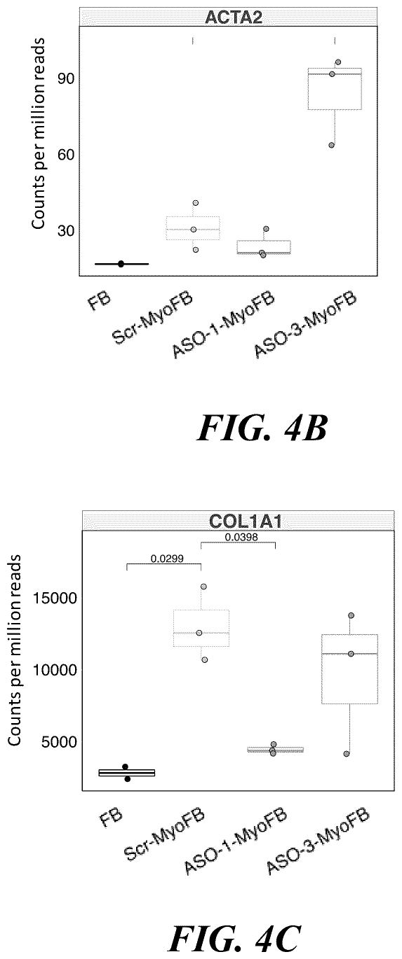

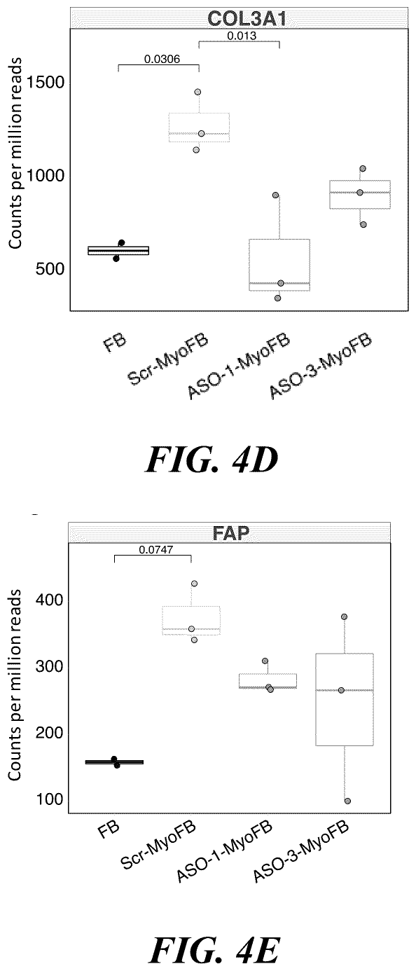

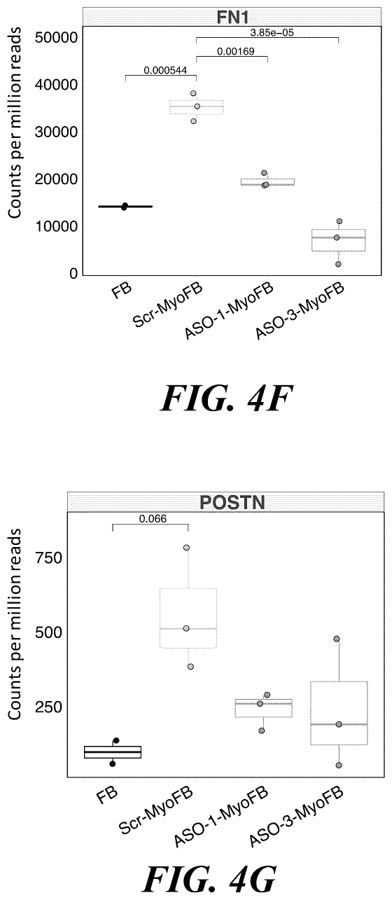

As shown in FIG. 1G, the provirus region, which is the full-length TE, is associated to LTR16C. Thus, ASO (ASO-3) was also designed to target LTR16C transcript, mapped to chr3: 45,818,736-45,818,762. As shown in FIG. 2A, ASO-3 is reverse complement of ASO-1, and ASO-3 can target a transcript transcribed from an opposite strand from a strand transcribing a transcript targeted from ASO-1. ASO-3 was an ASO with a 3-11-2 LNA GapmeR configuration and a phosphorothioate linkage between every nucleotide. To test whether ASO-3 can reduce the expression of LTR16C transcripts, myofibroblasts were treated with ASO-3 or ASO-Scr, and transcript expression levels were measured by qPCR with an LTR16C primer pair. The primer pair sequences were 5′-CCACAGTGCTAACCTGCTCA-3′ (SEQ ID NO: 12), and 5′-TCCCCCAAAAGCAAAACCCT-3′ (SEQ ID NO: 13). As shown in FIG. 4A, LTR16C transcript expression was decreased in the myofibroblast treated with ASO-3 compared to ASO-Scr.

### Fluorescence-Activated Cell Sorting (FACS) Analysis

FACS analysis of the expression FAP protein, a fibrosis marker, was analyzed on human lung fibroblasts (HLF) and their differentiated cells, MyoFB treated with ASOs. Medium was collected for each sample and cells were washed once with 1×PBS. Cells were dissociated by a 2 min incubation with TrypLE at 37° C. The collected medium was used to harvest its corresponding sample and the cells were spun down at 1500 rpm for 5 min. The supernatant was removed, and cells were resuspended in PBS (1×) and spun down again at 1500 rpm for 5 min. The cells were incubated in 100 μl FAP staining solution (FAB3715P antibody, R&D Systems, 1:100) or in IgG control staining solution (1:100) for 30 min at 4° C., protected from light. Samples were washed 2 times with FACS buffer followed by fixation by a 10 min incubation, 4° C., in 100 μl FACS FIX/Perm buffer. 2 more washes were done before the samples were recorded on LSR-I/II and analysed using FlowJo. As shown in FIG. 15, the proportion of FAP+ cells were decreased when using ASOs of interest as compared to scramble control. Additionally, FACS for apoptosis markers is performed to evaluate apoptosis upon treatment of ASO-1 or ASO-3. Viability and proliferation of HLFs with or without treatment of ASO-1 or ASO-3 is assessed with Incucyte live imaging machine.

Fibrosis Marker Analysis Via Quantitative PCR (qPCR)

Lung myofibroblasts, obtained from treating human lung fibroblasts (HLF) with TGFβ in serum free medium, were treated with ASO-1 or ASO-2 and expression levels of fibrosis markers, including ACTA2, COL1A1, COL3A1, FAP, FN1, and POSTN were measured. In the lung myofibroblasts that were treated with ASO-1 or ASO-2, statistically significant decreased expressions of fibrosis markers were observed, including ACTA2 (FIG. 2B), COL1A1 (FIG. 2C), COL3A1 (FIG. 2D), FAP (FIG. 2E), FN1 (FIG. 2F), and POSTN (FIG. 2G), compared to the lung myofibroblasts treatment with ASO-scramble control (ASO-Scr), as measured by qPCR. Lung myofibroblasts, obtained from treating human lung fibroblasts (HLF) with TGFβ in serum free medium, is treated with ASO-3 and expression levels of fibrosis markers, including ACTA2, COL1A1, COL3A1, FAP, FN1, and POSTN is measured by qPCR. Fibrosis marker analysis via RNA-sequencing (RNA-seq)

Additionally, RNA-seq was first carried out to confirm that the ASO-1 and ASO-2 down-regulated the fibrosis markers. As a negative control, ASO-scramble control was used to treat myofibroblast (Scr-MyoFB). As shown in FIGS. 2H-2M, while expressions of fibrosis markers were maintained high in myofibroblast treated with ASO-scramble control (FIGS. 2H-2M), expressions of fibrosis markers were decreased in myofibroblasts treated with either ASO-1 or ASO-2 (ASO-1 MyoFB or ASO-2 MyoFB). The decreased expression of fibrosis markers after treatment with ASOs indicates that inhibition of LTR16C had an anti-fibrotic effect.

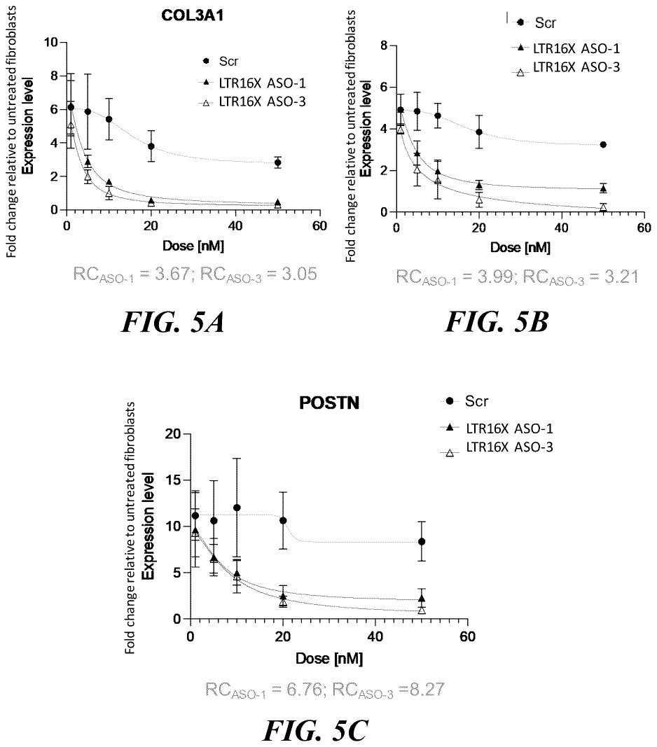

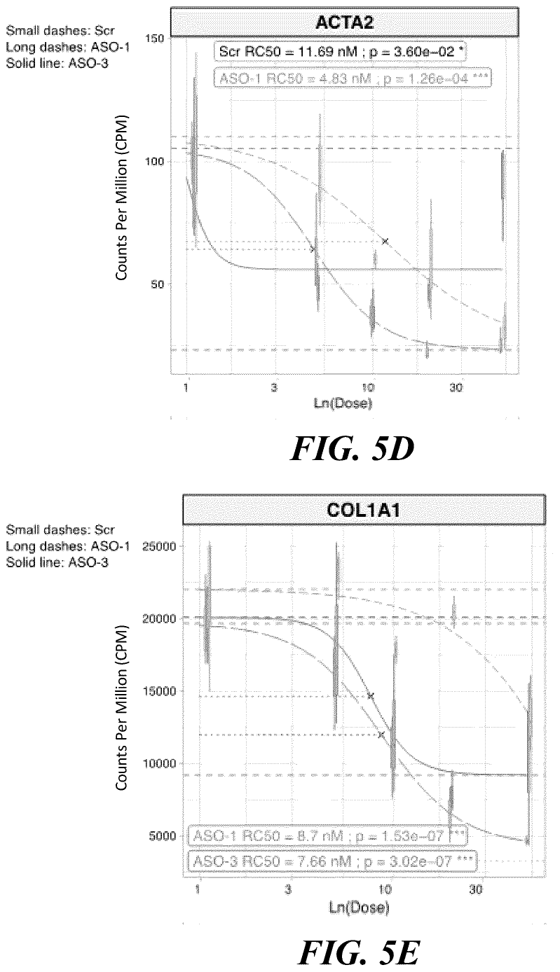

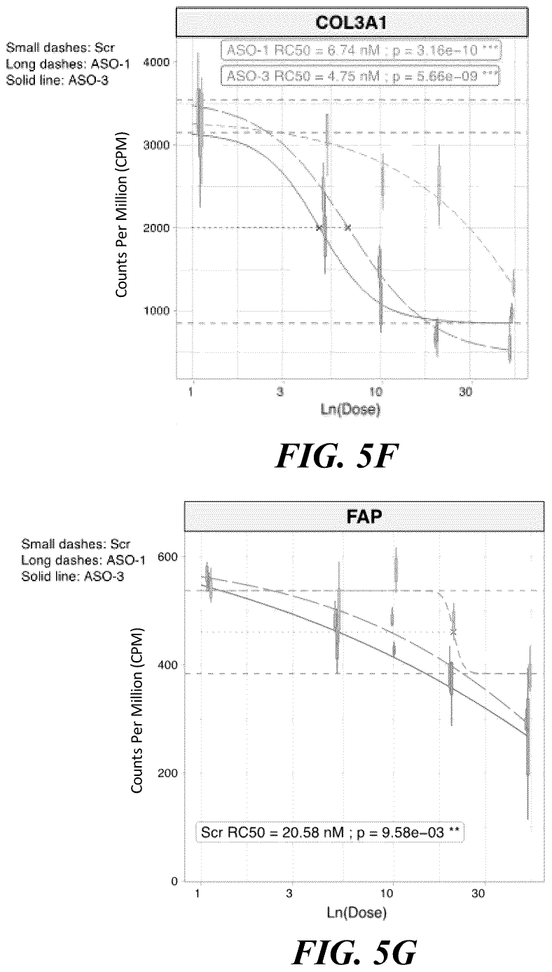

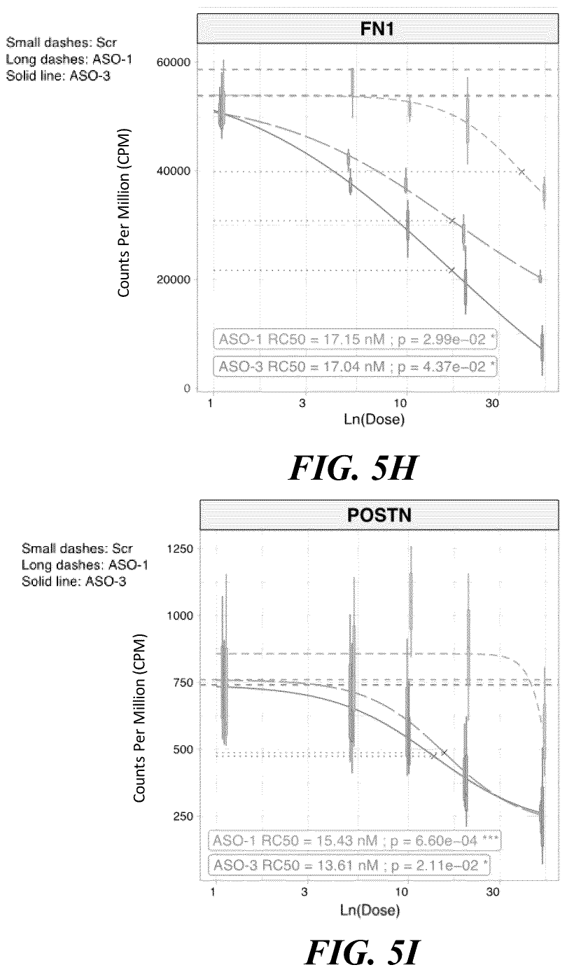

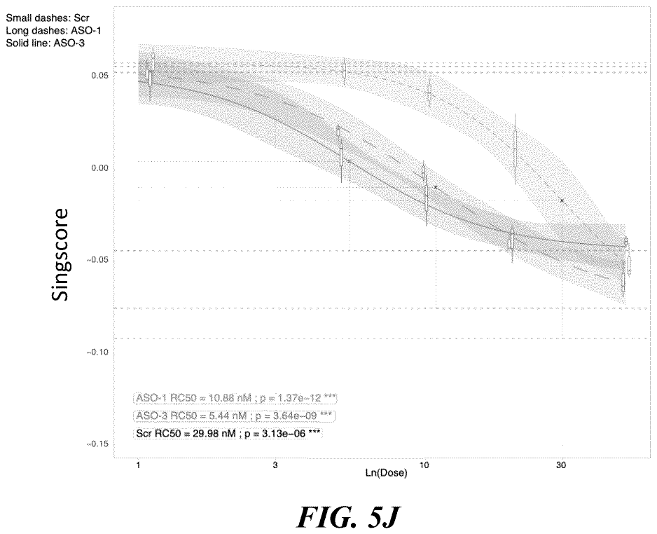

In addition, the quantity of LTR16C was measured from myofibroblasts treated with ASO scramble (Scr-MyoFB), ASO-1 (ASO-1-MyoFB), or ASO-3 (ASO-3-MyoFB) using RNA-seq. Untreated fibroblast (FB) was used as a negative control for fibrosis marker expression. Analysis of RNA-seq data of ASO-3-MyoFB or ASO-3-MyoFB showed decreased expression of fibrosis markers (e.g., ACTA2, COL1A1, COL3A1, FAP, FN1, POSTN) compared to Scr-MyoFB, as shown in FIGS. 4B-4G. Efficacy of the ASO-1 and ASO-3 in modulating fibrosis marker expressions (e.g., COL3A1, FN1, POSTN) were determined with myofibroblasts treated with 5 different doses (1 nM, 5 nM, 10 nM, 20 nM, 50 nM respective) of ASO-1 and ASO-3. As shown in FIGS. 5A-5C, myofibroblast treated with ASO-1 showed RC50 value of 3.67, 3.99, and 6.76 nM for COL3A1, FN1, and POSTN, respectively. Myofibroblast treated with ASO-3 showed RC50 value of 3.05, 3.21, and 8.27 nM for COL3A1, FN1, and POSTN, respectively. The curve for the ASO scramble control group (Scr) inflected to the right of the curves representing the ASO groups, suggesting myofibroblast treated with ASOs modulated fibrosis marker expressions compared to those treated with Scr. Dose-response analysis of RNA-seq data of myofibroblasts treated with ASO-1 or ASO-3 was performed to see changes in expression in fibrosis markers. As shown in FIGS. 5D-5I, dose-response analysis of the RNA-seq data revealed that as the dose of ASO-1 increased, there was decreased expression in fibrosis markers (e.g., ACTA2; RC50=4.83 nM, COL1A1; RC50=8.7 nM, COL3A1; RC50=6.74 nM, FN1; RC50=17.16 nM, POSTN; RC50=15.43 nM), suggesting the anti-fibrotic effect of the ASOs. Dose-response analysis of the RNA-seq data also revealed that as the dose of ASO-3 increased, there was decreased expression in fibrosis marker (e.g., COL1A1; RC50=7.66 nM, COL3A1; RC50=4.75 nM, FN1; RC50=17.04 nM, POSTN; RC50=13.61 nM). There results indicate that ASO-3 showed substantially similar efficacy with ASO-1 in modulating fibrosis-related gene expression.

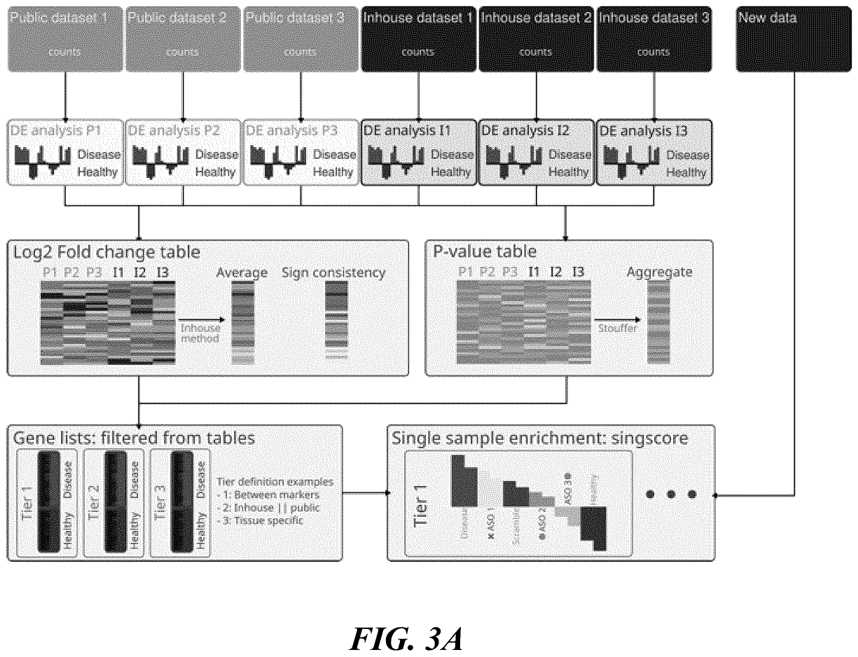

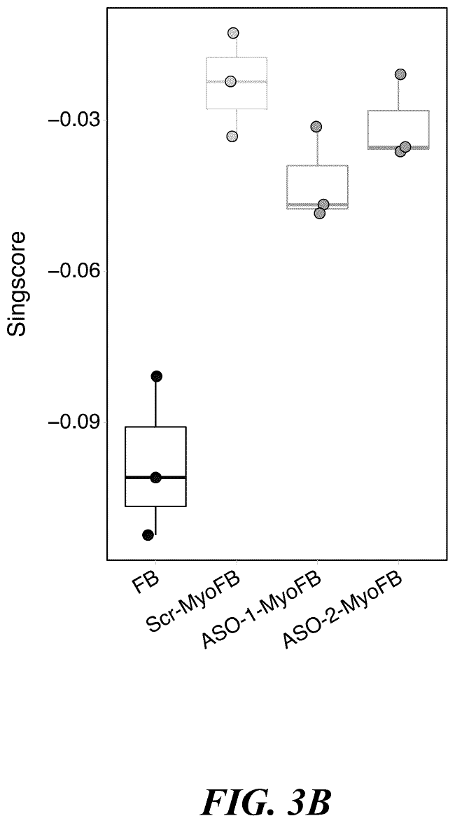

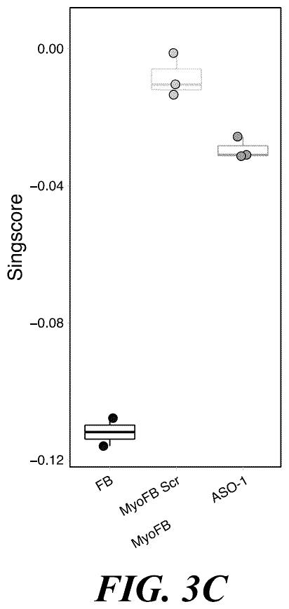

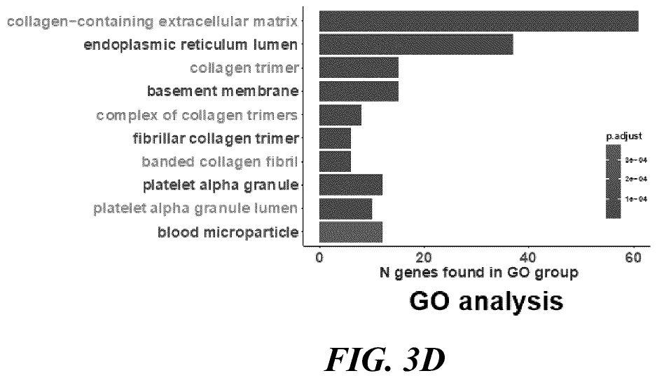

Transcriptional gene signature of idiopathic pulmonary fibrosis (IPF) was derived from in vitro and in vivo data sets, according to the workflow shown in FIG. 3A to validate the set or markers and to further evaluate the effect of inhibition of transcripts encoded by LTR16C. Differential expression (DE) analyses were conducted with sequencing datasets to identify genes that are upregulated and downregulated in healthy subjects versus subjects with IPF. The DE analyses were next aggregated for a meta-analysis to generate a transcriptional gene signature for IPF. Such generated IPF gene signature comprises 710 upregulated genes and 483 downregulated genes associated with IPF, as disclosed in Table 3. Gene ontology (GO) analysis of these genes revealed the genes' possible involvement in various cellular components, such as collagen-containing extracellular matrix, endoplasmic reticulum lumen, and collagen trimer, as shown in FIG. 3D. Therefore, the IPF gene set includes a group of genes that are stably differentially expressed across all these conditions and therefore constitute a core signature of IPF.

In order to analyze the gene expression pattern of the myofibroblasts based on the IPF signature gene set, Singscore, a rank-based gene signature scoring method, was implemented. In this example, gene expression patterns of myofibroblasts treated with ASO-1, ASO-2, or ASO-Scr control were analyzed using the IPF signature gene set with Singscore, wherein a higher score indicates that the pattern of gene expression in a sample is concordant with the pattern in the IPF signature gene set. As shown in FIG. 3B, myofibroblasts treated with ASO-1 or ASO-2 (ASO-1-MyoFB or ASO-2-MyoFB) showed decreased Singscore of IPF signature gene set compared to myofibroblast treated with Scr-MyoFB, indicating that ASO treatment on myofibroblasts reduced gene expression pattern concordance with IPF signature gene set. As a baseline, fibroblast (FB) gene expression was also analyzed using the IPF signature gene set with Singscore, which showed a lower Singscore for genes associated with IPF than MyoFB (FIG. 3B). Similar results were observed when gene expression of human lung cells treated with ASO-1 or ASO-scramble control (MyoFB Scr) for 24 hours and treated with TGFβ in serum free media for another 24 hours, were analyzed using the IPF signature gene set with Singscore, as shown in FIG. 3C.

Gene expression patterns of myofibroblasts treated with ASO-3, or ASO-Scr control was also analyzed using the IPF signature gene set with Singscore. Singscore of the average dose response for the entire IPF gene set also revealed decreasing score with increasing dosage of ASO-1 or ASO-3, with RC50=10.7 nM and RC50=5.44 nM, respectively, as shown in FIG. 5J.

### Analysis of Fibrosis Markers in Aged Mouse Lung Fibroblasts (MLFs) Via RNA-Seq

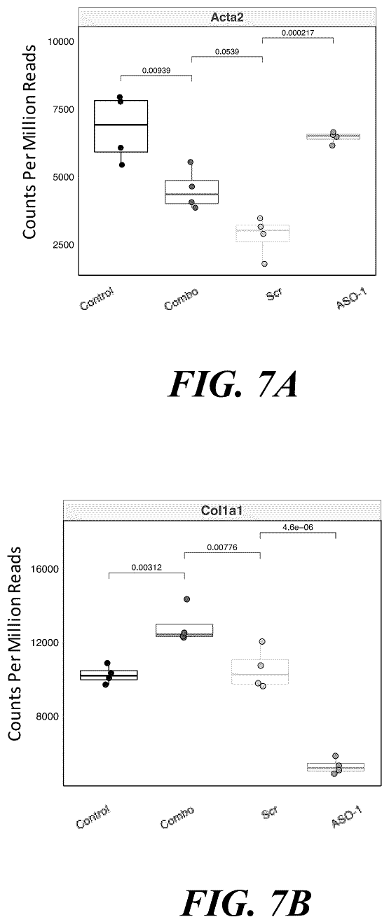

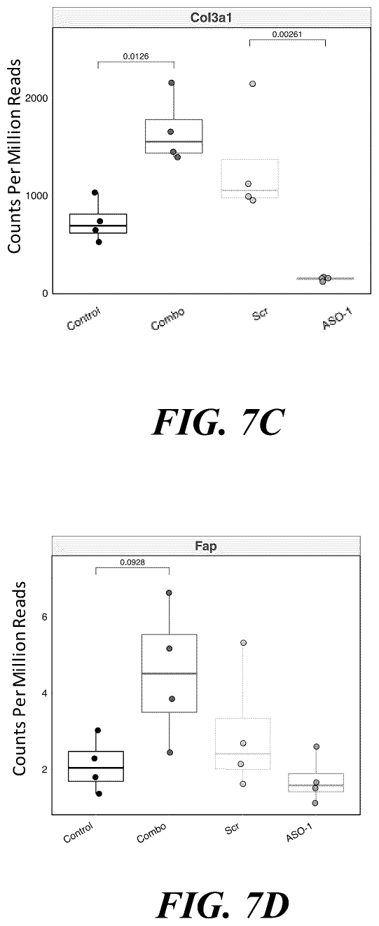

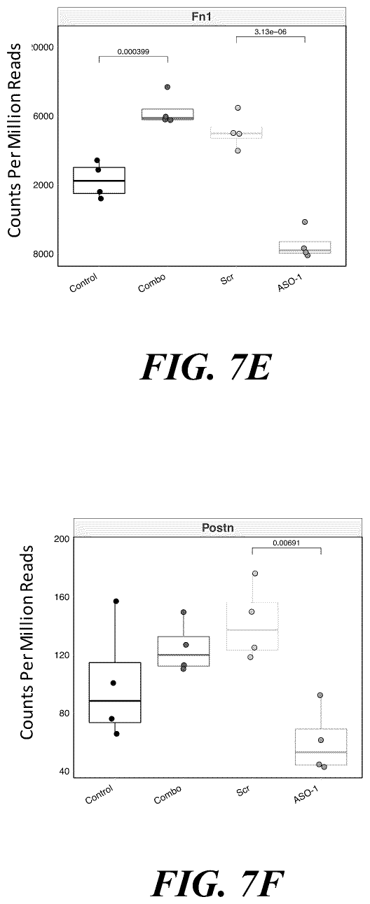

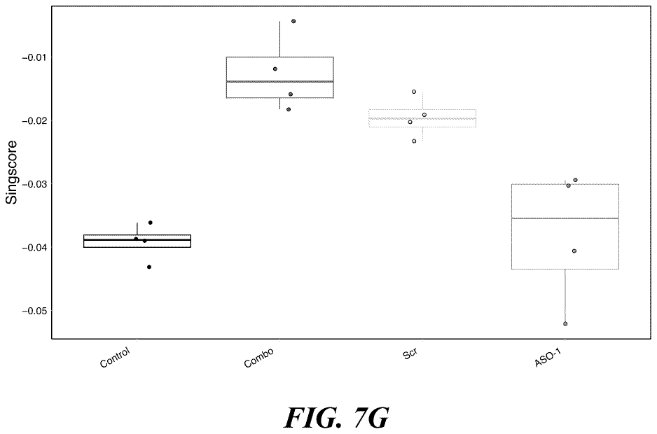

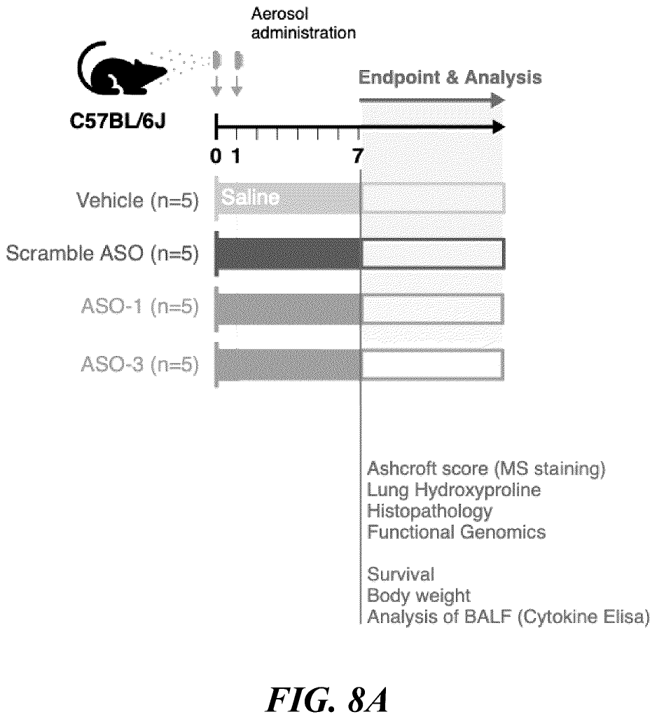

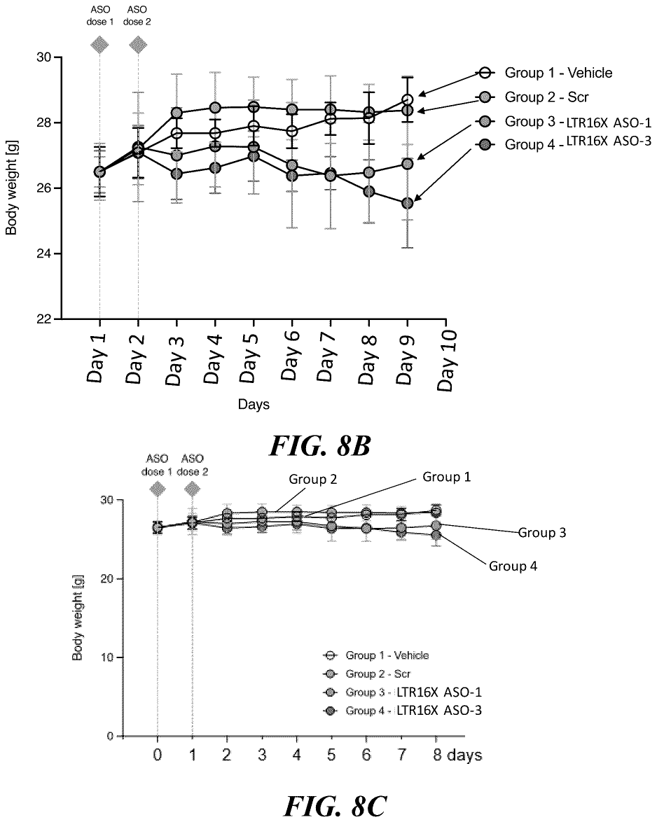

Aged mouse lung fibroblasts (MLFs) were treated with TGFβ and starvation, and then transfected with ASO-1 to see whether aged MLFs responded differently to ASO-1 treatment. Aged MLFs were isolated from snap-frozen lungs of 24-month old mice, and put in culture. The aged MLFs were cultured in DMEM with 10% Fetal bovine serum and 1% penicillin-streptomycin, and passaged with TrypLE. The MLFs were differentiated into myofibroblasts through serum removal and the addition of TGFβ for 48 hours after ASO-1 or Scramble (Scr) transfection. Transfection was performed with X-tremeGENE. RNA-seq analysis showed that ASO-1 modulated the expression of fibrotic markers (e.g., ACTA2, COL1A1, COL3A1, FAP, FN1, POSTN) in aged MLFs compared to aged MLFs treated with Scr (FIGS. 7A-7F). With ASO-1 treatment, aged MLFs showed statistically significant decreased expression of COL1A1 (FIG. 7B), COL3A1 (FIG. 7C), FN1 (FIG. 7E), and POSTN (FIG. 7F), compared to aged MLFs treated with Scr, showing that knock down of LTR16C led to downregulation of expression of anti-fibrotic genes. Fibroblasts (control) and untransfected fibroblasts treated with TGFβ and starvation (combo) were included to the RNA-seq analysis as controls to see that myofibroblasts have statistically significant increased expression of fibrosis markers (e.g., COL1A1, COL3A1, FAP, and FN1) compared to fibroblasts. Untransfected fibroblasts treated with combo were also included to compare with aged MLFs treated with Scr and combo to test whether Scr alone would affect gene expression. As shown in FIGS. 7A-7F, treatment with Scr did not exhibit statistically significant change in fibrosis markers (e.g., COL3A1, FAP, FN1, and POSTN) compared to untransfected myofibroblasts, suggesting that Scr can be used as a negative control for comparing effect of ASO-1 treatment. When comparing gene expression of ASO-1 treated aged MLFs to fibroblasts and untransfected myofibroblast treated with combo, expression of fibrosis markers showed decreasing trend. In addition to RNA-seq analysis for fibrosis marker genes, the human-derived IPF signature gene set was also analyzed with genes of ASO-1 treated aged MLFs. With Singscore scoring method, it was found that gene expression pattern of ASO-1 treatment on aged MLFs had reduced concordance with the gene expression pattern of the human derived IPF signature gene set, as shown by the lower Singscore compared to Scr-treated aged MLFs in FIG. 8G. This suggested that ASO 1 treatment affects gene expression pattern associated with fibrosis.

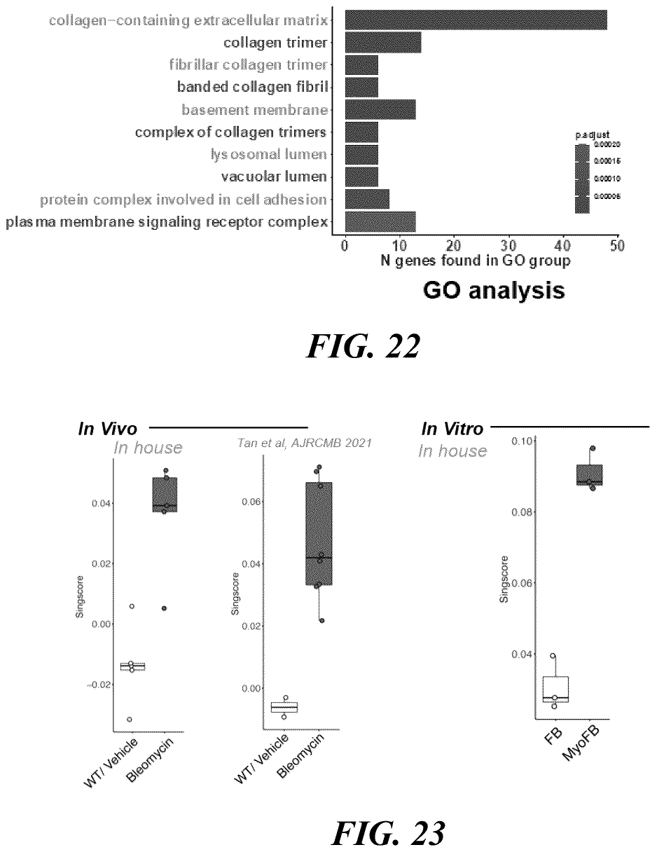

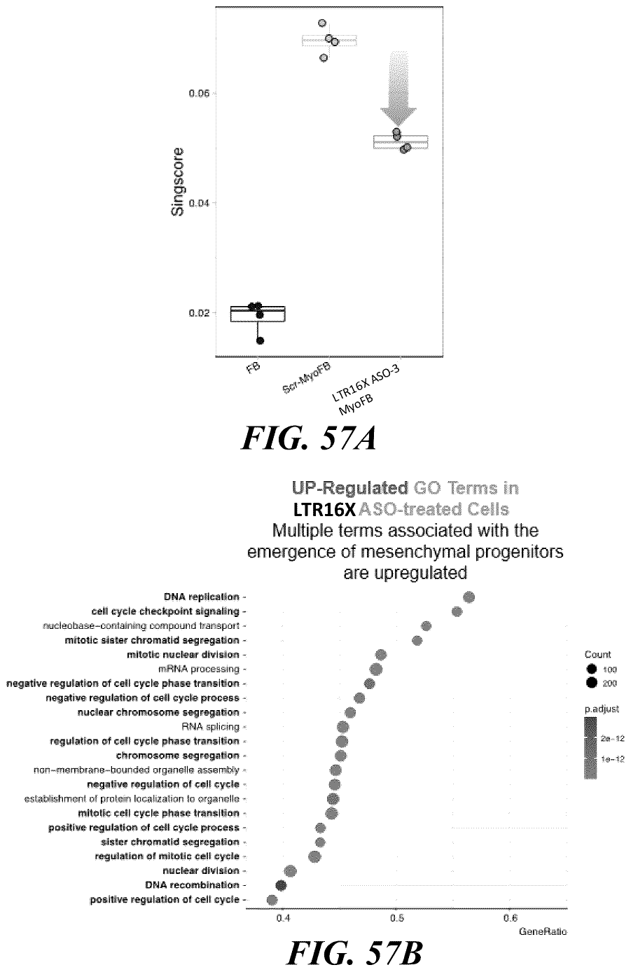

Another comprehensive readout, Bleomycin/MLF signature (see Table 5), was derived from a bleomycin mouse model in vivo study and publicly available data sets, similar to the workflow shown in FIG. 3A. The bleomycin/MLF signature was upregulated in bleomycin mice (in vivo) and in mouse lung myofibroblasts (in vitro), as shown in FIG. 23, confirming the association of the gene signature to in vivo and in vitro lung injury disease state. To investigate whether there is modulation of the bleomycin/MLF signature upon treatment with ASOs, bleomycin or MLF signature was measured via Singscore. Aged MLFs (e.g., from 24 month mice) were treated with TGFβ and starvation and transfected with ASO-1, ASO-3, or scramble control. As shown in FIG. 57A, with treatment of ASO-3, there was a decrease in bleomycin/MLF signature compared to treatment with scramble control. Gene ontology (GO) analysis of genes identified for the bleomycin or mouse lung fibroblast signature revealed the genes' possible involvement in various cellular components, such as collagen-containing extracellular matrix, collagen trimer, and fibrillar collagen trimer, as shown in FIG. 22. Furthermore, gene ontology analysis of upregulated genes upon treatment with ASO revealed multiple terms associated with the emergence of mesenchymal progenitors, suggesting possible cellular programming ability of ASOs. It is worth noting that IPF signature reflects some of the key features of senescence (e.g., ECM remodeling, TGFbeta signaling), suggesting tested ASOs impact senescence, which is also an aging hallmark.

TABLE 5
genes constituting Bleomycin/MLF signature

| Changes | |
|---|---|
| after | |
| bleomycin | |
| treatment | Gene names separated by spaces |
| upregulated | Ltbp2 Fn1 Chl1 Cdkn1a Serpine2 Slc37a2 Mgp Eln P4ha3 Ndnf P2ry6 Igf1 Col5a2 Brinp1 Apobec1 |
| | Cyp7b1 Ccn4 Serpina3i Nrp2 Lox C1qtnf6 Fmod Col5a3 Pdgfc Pdzrn3 Enpp1 Tnfsf18 Myo7a |
| | Tbc1d2 Ifi30 Chrna7 Chodl Lxn Il11 Rhod Deakd Sardh Gm6166 P2ry14 Enox1 Gm5424 Psrc1 |
| | Phlda3 Lama1 Ctsd Ctsc Gm49369 Slit2 Eda2r Sparc Col12a1 Nek6 Cxcl16 Casp3 Bax Gtse1 Pld3 |
| | Cthrc1 H2bc4 Akr1b8 Postn Ggta1 Acp5 Commd3 Pmaip1 Slc7a5 Gm37795 Loxl2 Cdh11 Lipa |
| | Fabp5 Adamts12 Hgf A930001C03Rik Dnm3os Hacd4 Ubtd1 Col1a1 1810058124Rik Coll1a1 |
| | Col8a1 Gm9844 Tnfrsf10b Ccl8 Tmem86a Rnf152 Ass1 P3h1 Capn6 RP24-87H1.13 Tmsb10 Gdf11 |
| | Mdk Syn2 Gm44751 Efna4 Cgref1 Thbs2 Nog Plod2 Mir6950 Mab21l3 Ctps Slc39a1 Fbn1 Adam10 |
| | Emilin1 Ccng1 Slc40a1 Psap Vcan Lyn Skap2 Kcnh1 Ak1 Prag1 Fzd1 Man2b1 Gja1 Cdkn2b |
| | Serpina3h Adam15 Unc93b1 Gng2 Pmepa1 Serpina3g Fuca2 Cyp51 H1f0 Hsd17b12 Rnf150 Pappa |
| | Nrg1 Mvd Ddias H1f2 ENSMUSG00002074901 Robo2 Ntper Col3a1 Edil3 Lgals3bp Masp1 Natd1 |
| | Zfp365 Sesn2 Thyn1 Ifi44 Cxcr4 Adgrg6 Orai2 Fam180a Trps1 Col16a1 Pycard Iigp1 Cd80 Lamb3 |
| | Ifi27l2a Kirrel3 Col18a1 Dhcr7 Mex3a Col1a2 Lyrm1 Eif4ebp1 Gm21451 Slc2a6 Mgmt Tspan6 |
| | Celf5 Nsdhl Pi15 Aldh1l2 Ak5 Ppp1r3d Art4 Fkbp1b Cd274 AA414768 Ighm Mrgpre Gm3788 |
| | Gm44741 2500002B13Rik Scube1 Hexb Lhfpl2 Tnfrsf1a Susd6 Zmat3 Cers6 Plscr3 Lrp12 Mmp2 |
| | Mdfic Atp6v1a Sox4 Pdgfb B4galt6 Aen Ap3s1 Lpcat3 Piezo2 Isg15 Spcs1 Osmr Tmem19 Fam20c |
| | Htatip2 Tmem178 Syngr2 Slc43a2 Bgn Ccdc80 Rab3il1 Pros1 Asah1 Dtx4 Apobr Grb10 Fam210b |
| | Lgals9 Syngr1 Tspo Emp1 Pik3r3 Pik3r3 Lpar6 Hmgcs1 Hexa Ddit4l B2m Cd302 Zfp992 Higd2a |
| | Slc29a3 Dbi Hvcn1 Scn3b Phex Slc8a1 Adamts7 Wls Ednra Flrt3 Hsd17b7 Znf41-ps Eif5a2 Shisa4 |
| | Mboat2 Cilp Ptk2b Mfap5 Nfkbie Lrp2 Pstpip1 Msmo1 Tmem45a Fam217b Gm49337 Pcdhb7 Fdps |
| | Tmem192 Dennd2c Aldoc Kene4 Serf1 B430305J03Rik Ogdhl Ano3 Oasl1 Trarg1 Tgfbi Sdc1 Ifngr2 |
| | Nlgn2 Kif26b Mrc2 Tmem167 Itgb5 Loxl1 Rnf19b Serpinb9 Dpy19l1 Bcl11a Pidd1 Slfn8 Lipg |
| | Trim30a Elovl1 Ptprv Mfap4 Prn Pdk3 Scx Gpx7 Tlr6 Scd1 Gpc4 Ifitm2 Nfasc Trim30d Il7 Hhat |
| | Idi1-ps1 Pgls Ackr3 Arntl Rab29 Eola1 Gpr141 Fam110c Fstl1 Pqlc3 Tgfbr2 Nedd9 Scarb2 Exoc4 |
| | Ctsl Rap2b Slc19a2 Pdgfrb Fads2 Ndrg1 Hmgcr Tpp1 Dpysl3 Slc25a1 Pvt1 Gm20547 Slc31a2 Nhs |
| | Tbx5 Slc2a9 Cpped1 Adamts9 Bdnf Idi1 Cercam Pycr1 Tpmt Manba Svep1 Cdk19 Scd2 Gpr146 |
| | Ehd3 Ace Marchf3 Heyl Ism1 F11r Rflnb Trp53inp1 Stard4 Sulf1 Csgalnact1 B3gnt8 Narf Rnf13 |
| | Itga1 Samd12 Adamts15 Bmp5 Adam23 Gadd45a Tcn2 Irf7 Sbk1 Gria3 Cpne8 Tmem229b Rnf122 |
| | Zmynd15 Psmb9 Elovl6 Itgal Bst2 Dhx58 Pcdhb14 Inka1 Zfp688 Pstpip2 Mras Apaf1 Fat1 Ahr |
| | Pitpnc1 Acox3 Kit Jam2 Adamts2 Dher24 Ddx58 Tmem98 Pag1 Ebp Sesn3 Erap1 Scp2-ps2 |
| | Gm43738 Cmklr1 Fas Abhd4 Matn4 Parp9 Nol4l Oas2 Sqle Fads1 Pcdhb9 Ptgfrn Ckb |
| | F830016B08Rik Unc119 Cpt1a Pdgfrl Heph Dtx3l Vwa5a Slc44a2 Ei24 Fstl3 Scn1b Tenm4 Itfg1 |
| | Dach1 Ifih1 Ubl3 Laptm4a Dpysl2 Plscr2 Lzic Mdm2 Inka2 Cln8 Hs6st1 Arhgef6 Man1c1 Rnf169 |
| | Siva1 Tdrd7 Tor3a |
| downregulated | Wnt2b Fez1 2010310C07Rik Ldb3 Xirp1 P2rx1 Synm Col4a6 Epha1 Aldh3a1 Pdlim3 Lmod1 Kenk2 |
| | Alms1 Cep112 Smoc1 Shank2 Map3k19 Kcnd3 Xk Ccdc15 Gm25117 Acte1 Gm48958 Pnck Ppm1e |
| | mt-Tm Atcayos mt-Tn Cep295nl Slc6a17 ENSMUSG00000119662 Gm15958 Tgfbr3 Usp54 Igfbp5 |
| | Clip1 Grem2 Por Wdr66 Sord Syne2 Pcolce2 Tns1 Map1b Lgr6 Lncpint Otud1 Tent5c Rhobtb1 Chka |
| | Kank1 Armc2 Spice1 mt-Tp Thsd4 Ift81 Hsph1 2700078F05Rik Kcnmb1 Gipc2 Srsf7 Gm37018 |
| | Cnn1 Patj Tnnt3 Bambi Gm34455 Bves Asb2 mt-Tt Sh3bgr Dnajb4 Wnk4 Mylk2 Gm5844 Dnaja1 |
| | Ptgs2os2 mt-Tq Snord22 Gm5176 Hspa1a Sned1 Phldb1 Ppp1r12b Bcl7a Fgfr2 Usp2 Plekha6 Nav2 |
| | Sphk2 Phlpp1 Met Sox13 Rian Ccp110 Foxcl Lnx1 Ugt1a5 Diras2 Myom1 Cdc42ep3 Osbpl3 Ugt1a2 |
| | Rab3ip Pik3c2b Sfxn2 mt-Cytb Tyro3 Ugt1a1 Sclt1 Ralgps2 Junos Samd4 Dtna Sorbs1 Proser2 |
| | Tmem107 Dgat2 mt-Nd6 Ccdc81 Nr4a1 Cfap43 Luc7l3 Kcnk5 Myocd Twist2 Exosc7 Hsp90aa1 |
| | Mustn1 Il15ra Paxip1 Eid3 Gm12346 Srf 1110046J04Rik Mir6901 Enkur Greb1l Proser3 Gm49358 |
| | Nr4a2 Chn2 Pxmp2 Bag2 Nat14 5S_rRNA Efcab5 Arid3b Me3 Tex9 Nr6a1 Alms1-ps2 Arc Cdc25b |
| | Smad9 mt-Tl2 Ier2 Cracd 2310030G06Rik Slco1a5 Lancl3 Gm37258 Gm4419 Cenpv Gm37949 |
| | Cacnb4 Gm37677 Arl4d Acsl1 Pde4dip Adamtsl4 Plekhg3 Rock1 Prodh Cgnl1 Setd1b Galnt16 |
| | Adcyap1r1 Slc23a2 Epdr1 Tst Ing5 Traf3ip2 Ugt1a6b Prkag2 Itih2 Zc3h13 Nt5dc3 Nt5dc3 Wdr38 |
| | Myo1h Fgfr3 Pdk4 Usp36 Mdm1 Mns1 Spen Ftx Prepl Tmod1 Cep126 Atp2a2 Htr1b Akap12 Zbtb20 |
| | Trim65 Cap2 Mtcl1 Cep85l Otud3 Cep290 Cnst Pcnt Hid1 Fbxo30 Lcor Ugt1a10 Ahsa2 Safb Ugt1a8 |
| | Mtus2 Uap1 B930086L07Rik Ugt1a9 Rhou Odf2l Sipa1l3 Fam171b Tra2a Sik1 Aven Carmn Xdh |
| | Irag1 Sltm Hnrnph1 Eno3 Xntrpc Lrrc1 Snapc4 Cldn1 Slc38a1 Mat2a Ppargc1a Sf3b2 Katnal2 Akap6 |
| | Rsrc2 Safb2 Avpr1a Hip1r Gm17823 Gramd1c Dusp10 Kif24 Igsf9 Chd1l Car8 Ccdc85b Nrn1 |
| | 1810059H22Rik Hmgn5 Syne3 Sorbs2 Cobll1 Rbm28 Chst7 Gm38708 Cep128 Xndcl Slc38a4 |
| | Gm4876 Dmpk Fzd3 Rbpms2 Smtn Gm15411 Sms Arhgdig Pawr Ccdc134 Hspa12a Ccdc85c Dsc2 |
| | Nr4a3 Wdr90 Cdr2 Rnf207 Enkd1 Cntnap1 Ccdc138 Lpin3 Ccdc120 Cntrl Dusp8 Cplx2 Myl9 Ahctf1 |
| | Rbpms Fam149a Enah Trim56 Pptc7 Rif1 Abcc1 Soga1 Frmd6 Ttll4 Mtr Hdac5 Arhgap10 Csnk1e |
| | Rdm1 4930523C07Rik Ankrd26 Fbln1 Slmap Fhdc1 Urb1 Oat Acat1 Tm4sf1 Spry2 Socs7 Map1a |
| | Plekha5 Hirip3 Gm12940 Slc43a1 Slc29a1 Zdhhc14 Epb41l3 Srsf10 Cep43 Cfap91 Brd9 Ktn1 Sgms2 |
| | Lzts2 Dlg4 Meis1 Esrp2 Pkdcc Tmem201 Gm42715 Ssx2ip Dtnb Opcml Wee1 Gon4l Gm43552 |
| | Phf20l1 Slc38a2 Htatsf1 Acta2 Mdm4 Mgst2 Sh3bp5 Akrlc14 Net1 Afg1l Dnaaf2 Sfswap Srek1 |
| | Supt5 Unc45a Mapkbp1 Smad3 Itpr3 Echdc3 Cnot3 AW554918 Chordc1 Frem2 Rbm25 Kmt2b |
| | Fam118a Rhot2 Ilrun Stk40 Setd6 Gm37084 Ano1 Sart1 Nktr Pde4a Brd8 B4galt3 Gm21596 Faxc |
| | Top1 Crebzf Vapb Akap8 Baz1b Gabpb2 Clip4 Qtrt2 Maob Fgfr1 Htra1 Urgcp Bdp1 Taf3 F3 Pde3a |
| | Mdc1 Rock2 Ncoa5 Bclaf1 Luc7l Smc3 Srcap Nfx1 Polg Gle1 Nmt2 Fryl Tpm2 Dnajc21 Plagl2 |
| | Fam210a Cebpz Rasa2 Atl2 Map3k20 Pde3b Rbm14 Cdc42bpa Crlf3 Wdfy1 Pank2 Arhgef10l |
| | Micall1 Gxylt1 Tmpo Slf1 P3h2 Ehmt1 Eif3a Tspan11 Nsun5 Kmt2d Cep135 Azi2 Tnik Diaph1 |
| | B3galt1 Srgap3 Mir17hg Fam117a Rassf3 Cabin1 Dusp16 Ccdc141 Ppig Ddx23 Usp10 Tmcc3 Rnf20 |
| | Zmat1 Emc1 Otud4 Afdn Spop Esyt2 Mylk Mef2d Smurf2 Anapc5 Tbc1d16 4930402H24Rik Sik3 |
| | Sf1 Nqo2 Ppp4r2 Jmjd6 Sfpq Ccdc167 Srsf4 Fzd7 Fip1l1 Srsf2 Oxsr1 Mical2 Hnmnpa2b1 Nin Smad7 |
| | Uspl1 Swap70 Camsap1 Hmgxb3 Supt6 Prps1l3 Scaf4 Nop14 Cipc Arid5b Cdk8 |

Fibrosis Marker Analysis Via Single Nucleus RNA-Sequencing (snRNA-Seq)

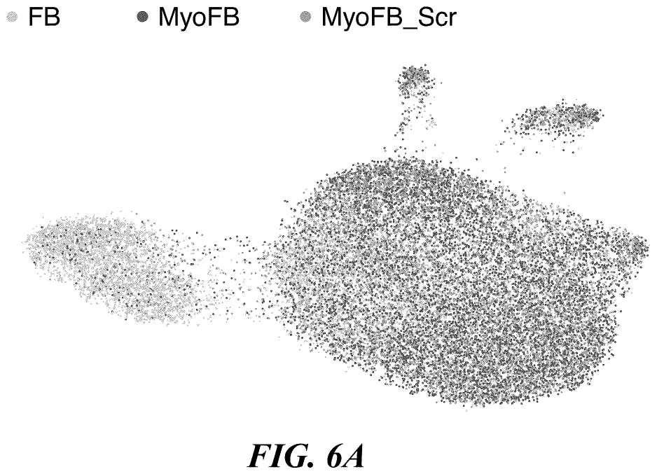

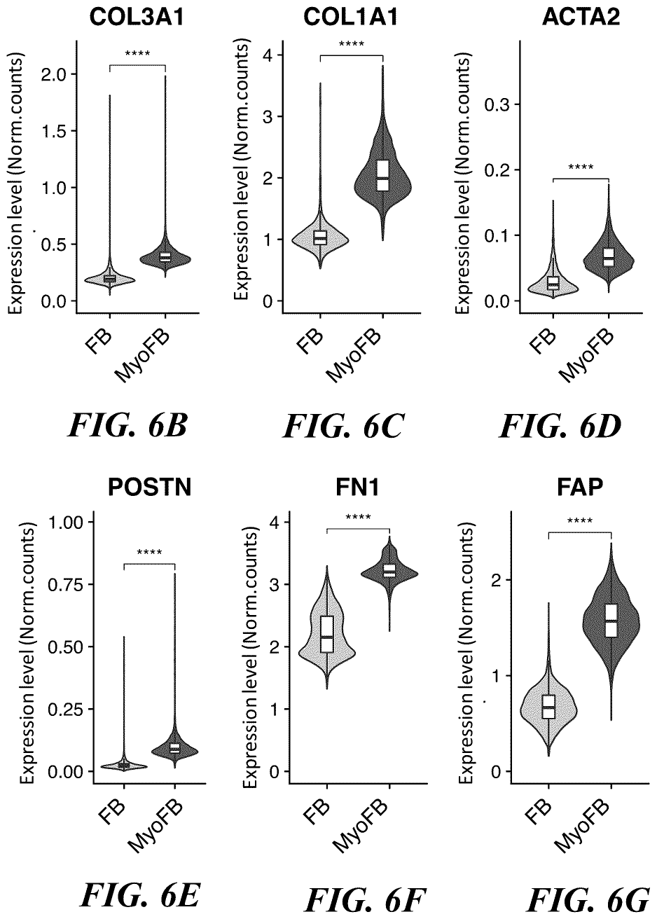

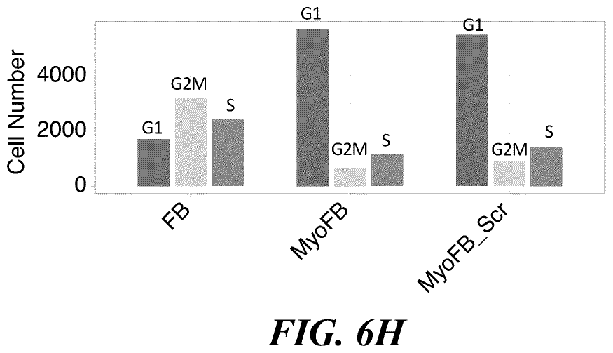

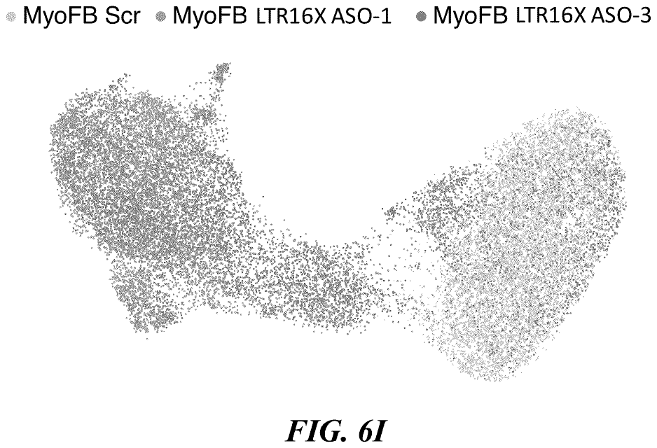

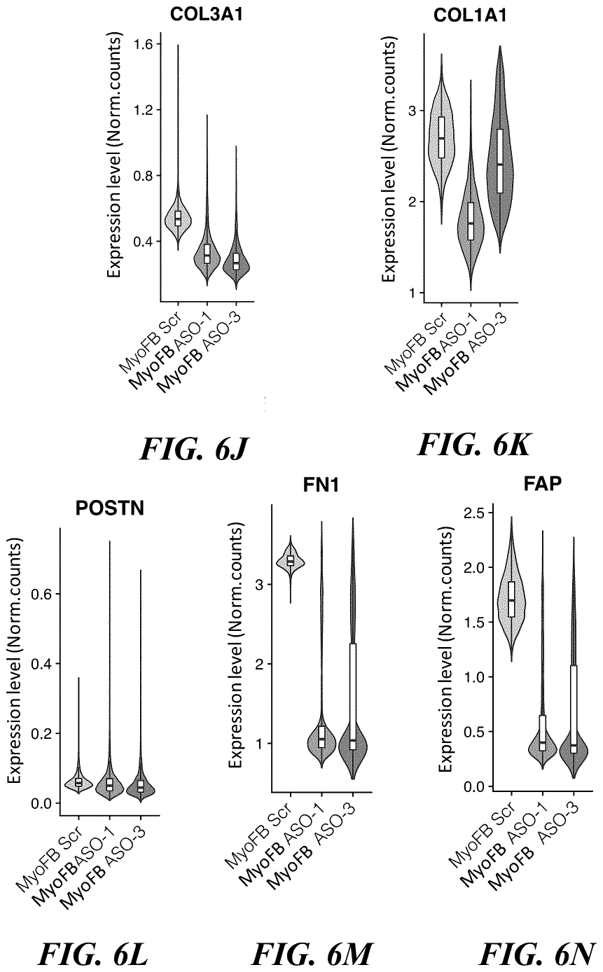

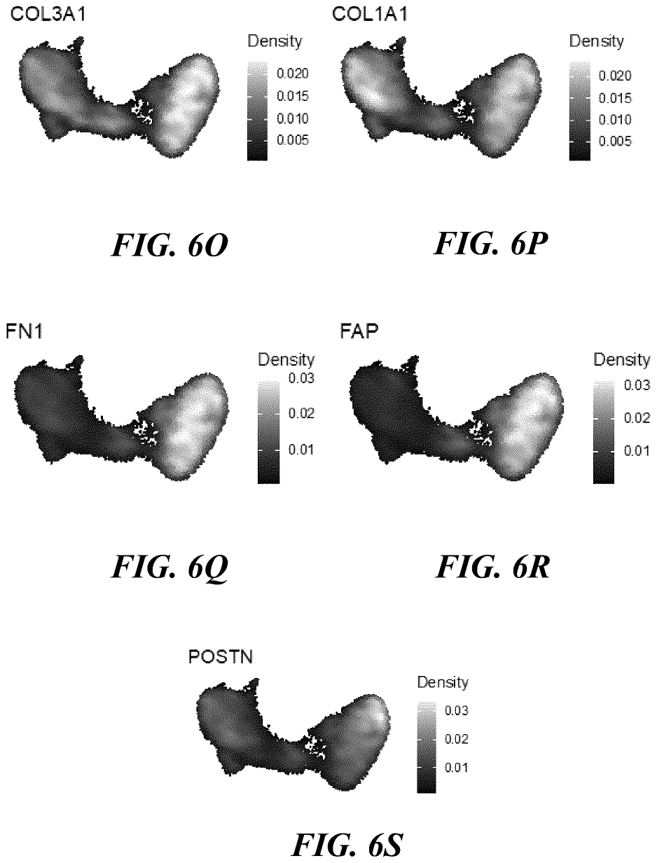

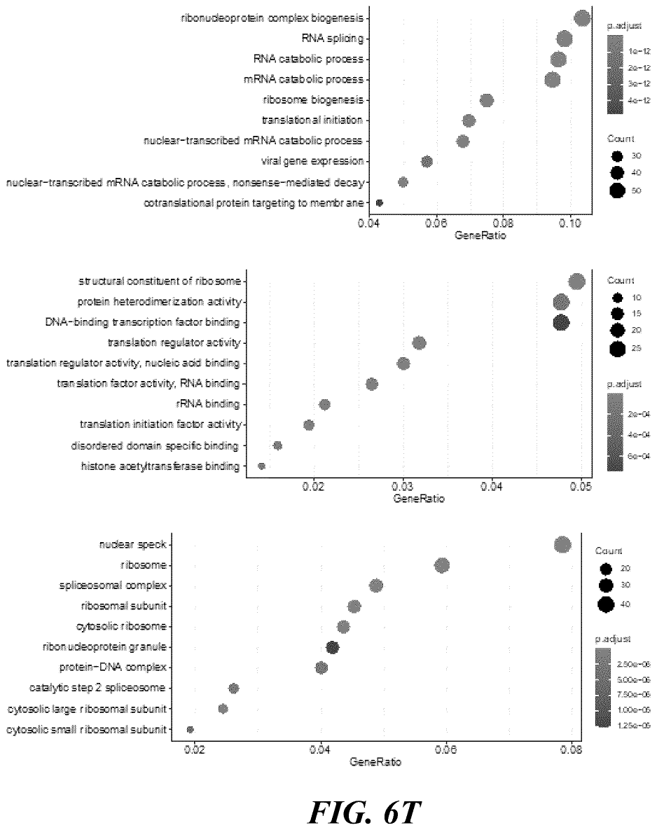

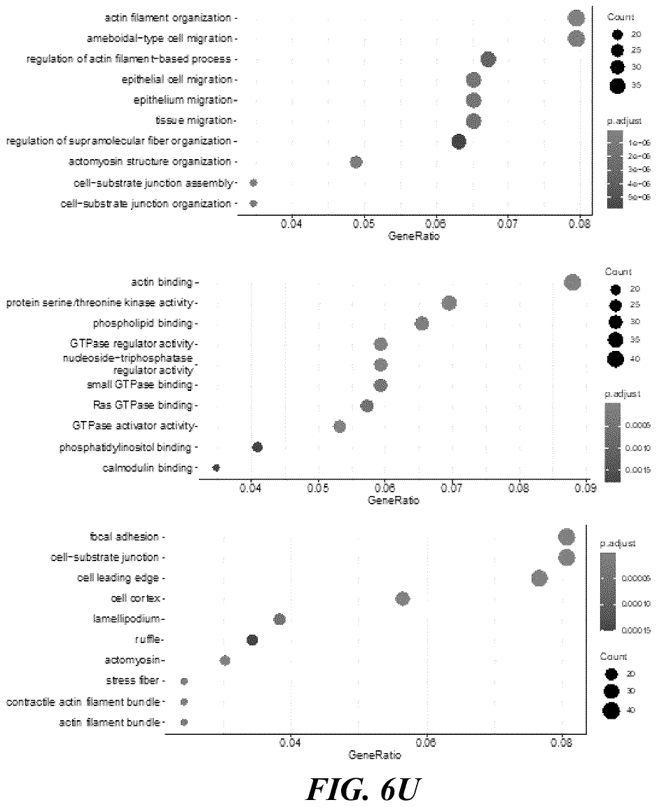

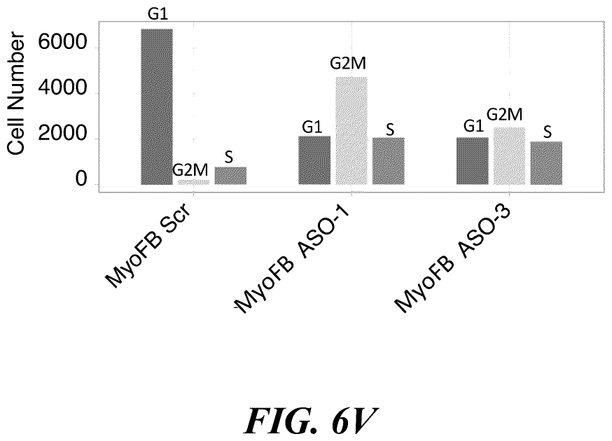

In addition to the bulk-RNA-seq, single nucleus RNA-sequencing (snRNA-seq) was performed to determine the effect of ASOs in gene expression of fibrosis markers at the single nucleus level. First, fibroblasts (FB), myofibroblasts (MyoFB), and myofibroblasts treated with ASO-scramble control (MyoFB_Scr) were processed with Chromium X at 4 million cells per condition to verify that FB and myofibroblast represents different cell populations and to test whether transfection with scramble control changes cell state of MyoFB. Uniform Manifold Approximation and Projection (UMAP) analysis of snRNA-seq data showed two separate clusters, one representing FB and the other representing MyoFB, confirming that FB and MyoFB cell populations had different gene expression profiles (FIG. 6A). In addition, snRNA-seq data showed that transfection with scramble control did not substantially change the gene expression profile of MyoFB, as distribution of untransfected MyoFB and MyoFB-Scr overlapped on the UMAP (FIG. 6A). snRNA-seq data analysis also showed that MyoFB had increased expression of fibrosis markers (e.g., COL3A1, COL1A1, ACTA2, POSTN, FN1, and FAP) relative to FB, further verifying the difference between FB and MyoFB cell populations (FIGS. 6B-6G).

Subsequently, the snRNA-seq of MyoFB-Scr was compared to that of myofibroblasts treated with ASO-1 or ASO-3 (MyoFB ASO-1, MyoFB ASO-3). UMAP analysis of snRNA-seq data showed that the majority of cells in MyoFB ASO-1 and MyoFB ASO-3 populations were overlapped and localized in the same or substantially similar cell state (left group of cells in FIG. 6I), while the majority of cells in MyoFB-Scr population was localized in a distinct cell state (right group of cells in FIG. 6I).

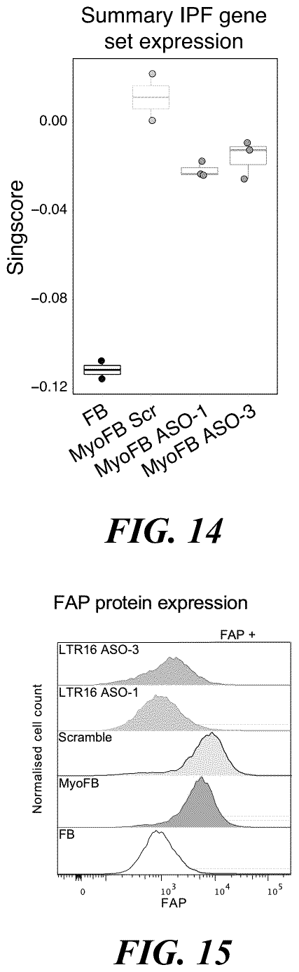

Expression of fibrosis markers in these cell populations was also analyzed with snRNA-seq data. As shown in the violin plots (FIGS. 6J-6N) of the snRNA-seq data, treatment of ASO-1 and ASO-3 to the myofibroblast decreased the expression of fibrosis markers (e.g., COL3A1, COL1A1, POSTN, FN1, FAP) compared to treatment with MyoFB Scr, suggesting an anti-fibrotic effect by inhibiting LTR16C transcript expression or activity. snRNA-seq data was also presented as a gene expression heat map. Similar to the gene expression pattern seen in the violin plot representation, the gene expression heat map showed that the gene expression density of fibrosis markers (e.g., COL3A1, COL1A1, FN1, FAP, and POSTN) was lower than that of cells treated with ASO-Scr (FIGS. 6O-6S). Furthermore, 702 upregulated genes and 541 downregulated genes upon ASO-1 were identified with snRNA-seq data. These genes were subjected to gene set enrichment analysis (GSEA) to associate functions to the group of upregulated and downregulated genes (FIG. 6T and FIG. 6U, respectively). GSEA revealed potential fibroblast functions, such as actin filament organization, epithelial cell migration, focal adhesion, and stress fiber as shown on the y-axis of FIG. 6U. X-axis measured the GeneRatio, which is the percentage of genes in the corresponding GSEA term that are found to be differentially expressed. (FIG. 6U). In addition, with Singscore scoring method, it was found that gene expression pattern of ASO-1 or ASO-3 treatment on myofibroblast had reduced concordance with the gene expression pattern of the human derived IPF signature gene set, as shown by the lower Singscore compared to Scr-treated myofibroblast (FIG. 14).

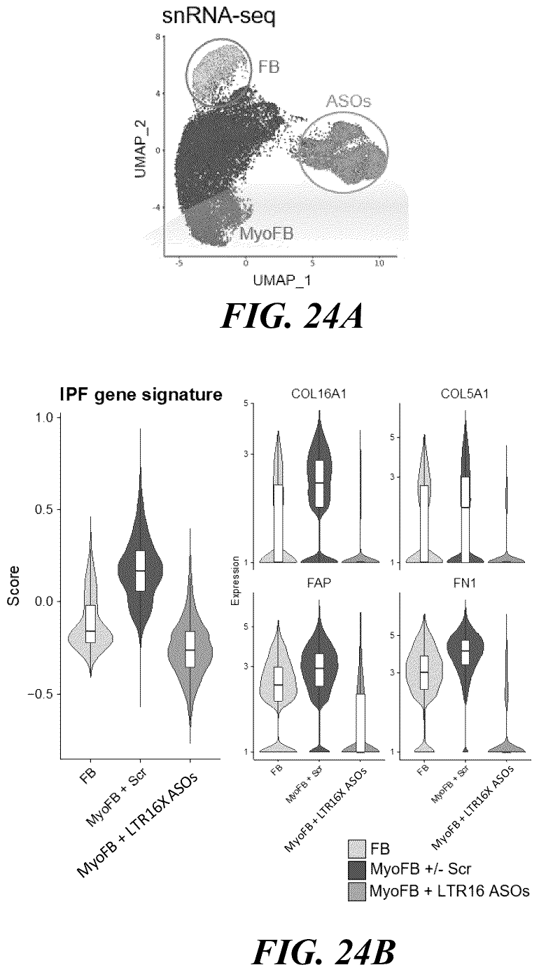

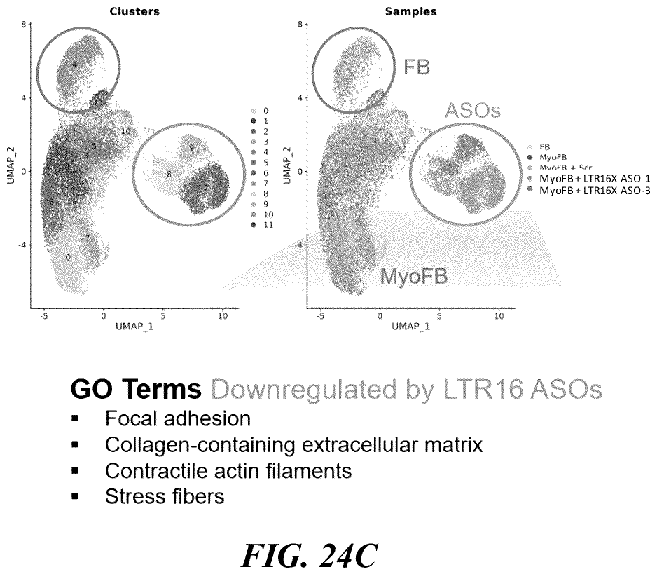

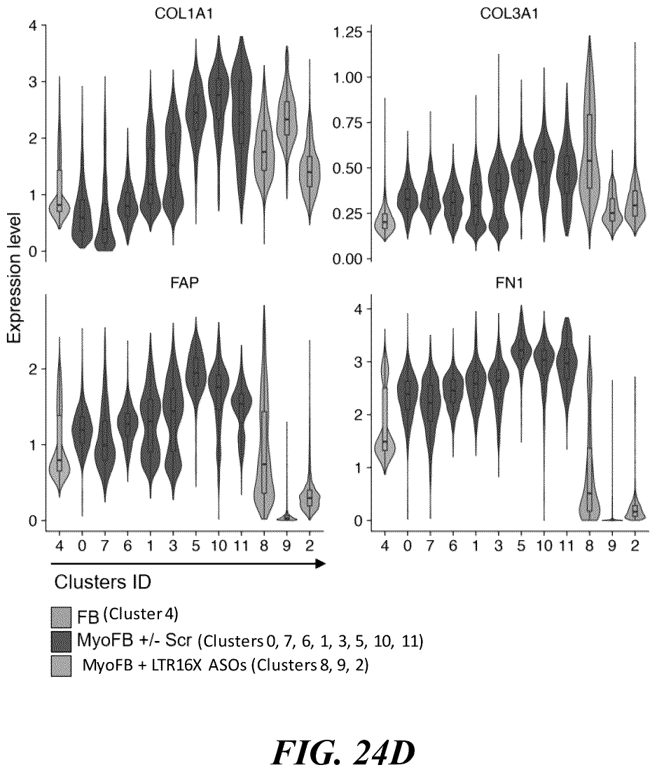

Another run of snRNA-seq was performed with human lung fibroblasts (HLF) treated with ASO-scramble control (MyoFB_Scr), ASO-1 (MyoFB+ASO-1) or ASO-3 (MyoFB+ASO-3), upon treatment with TGFβ plus serum starvation. As shown in FIG. 24A and FIG. 24C, snRNA-seq revealed distinct clusters representing fibroblasts, myofibroblasts, and ASO-transfected myofibroblasts. Gene Ontology (GO) analysis of genes differentially expressed in ASO-transfected myofibroblasts revealed that downregulated genes are associated with focal adhesion, collagen-containing extracellular matrix, contractile actin filaments, and stress fibers (FIG. 24C). Idiopathic pulmonary fibrosis (IPF) gene signature and expression of fibrosis markers in these cell populations were also analyzed with snRNA-seq data. As shown in FIG. 24B, myofibroblasts treated with ASO-1 and ASO-3 showed decreased Singscore of IPF signature gene set compared to myofibroblast treated with scramble control, indicating that inhibition or downregulation of LTR16C transcript by ASO treatment on myofibroblasts reduced gene expression pattern concordance with IPF signature gene set. Furthermore, as shown in the violin plots (FIG. 24B) of the snRNA-seq data, treatment of ASOs decreased the expression of fibrosis markers (e.g., COL16A1, COL5A1, FAP, FN1) in myofibroblasts compared to treatment with scramble control. Clusters 2, 8, and 9, which correspond to myofibroblast treated with ASO-1 or ASO-3 also showed decreased expression level of fibrosis markers (e.g., COL1A1, COL3A1, FAP, FN1) compared to other clusters that correspond to myofibroblast treated with scramble control (FIG. 24D).

### Broader Targeting by ASO-1 and ASO-3

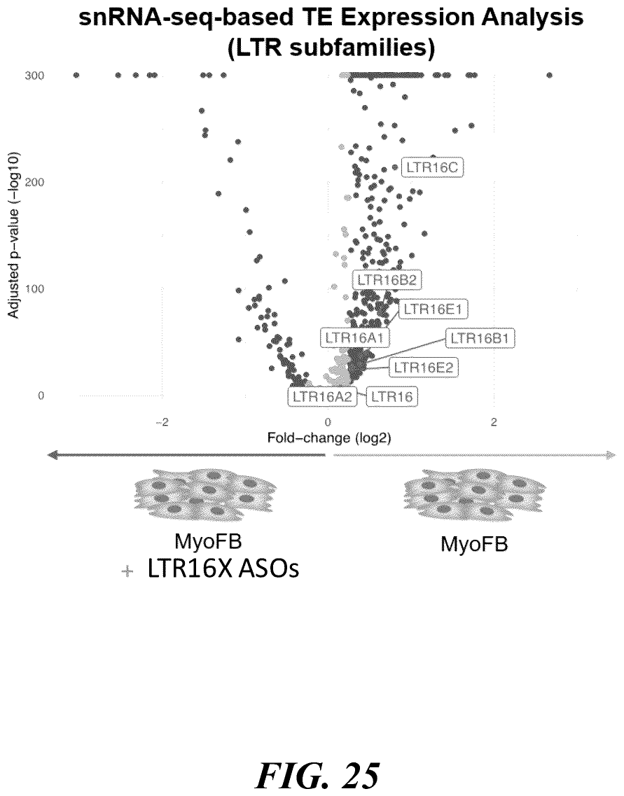

As described, single nucleus RNA-seq (snRNA-seq) was performed on human lung fibroblasts (HLF) treated with TGFβ plus serum starvation for differentiation to myofibroblasts. Cells were treated with ASO-scramble control (MyoFB+Scr, Scr, MyoFB Scramble), ASO-1 (MyoFB+ASO-1) or ASO-3 (MyoFB+ASO-3). Analysis of the generated snRNA-seq data for transposable element expression revealed that upon treatment with ASOs (ASO-1, ASO-3), there was a downregulation of target transcripts encoded by LTR16 subfamilies compared to myofibroblasts with no ASO treatment, as shown in FIG. 25.

Furthermore, a mismatch analysis of ASO-1 and ASO-3 to various TE subfamilies was performed. As shown in FIG. 2N, about 1222 integrants that belong to several subfamilies (collectively refer to as LTR16X subfamilies) were identified (see Table 4). As shown in Table 4, the targeted integrants (i.e., TEs from different loci) belong to LTR16, LTR16A, LTR16A1, LTR16A2, LTR16B, LTR16B1, LTR16B2, LTR16C, LTR16D, LTR16D1, LTR16D2, LTR16E1, LTR16E2, HERV16, ERV3-16A3_LTR, and ERV3-16A3_I subfamilies. All of the identified elements were aligned to where the ASO was found (indicated as the target site on FIG. 2N). In addition, upon motif scanning for transcription factors along the obtained consensus, it was revealed that ASO-targeted elements bear motifs for lung fibrosis-specific transcription factors, such as EBF1 and SPI1. Gene ontology (GO) analysis of protein-coding genes closest to the targeted LTR16 elements highlighted their role in TGF-beta signaling network and fibrosis. These data showed that the designed ASOs target the transcripts encoded by the LTR16X subfamilies.

TABLE 4
genomic coordinates of integrants ASO-1 or ASO-3 targets

| Chromosome | coordinates | strand | subfamily |
|---|---|---|---|
| chr1 | 54104842-54105131 | + | ERV3-16A3_I-int |
| chr6 | 164080103-164080725 | − | ERV3-16A3_I-int |
| chr1 | 209445313-209445890 | − | ERV3-16A3_I-int |
| chr14 | 96636197-96636826 | − | ERV3-16A3_I-int |
| chr15 | 81573996-81574370 | + | ERV3-16A3_I-int |
| chr1 | 71821838-71823069 | + | ERV3-16A3_I-int |
| chr19 | 33783927-33784880 | + | ERV3-16A3_I-int |
| chr20 | 14624057-14624604 | + | ERV3-16A3_I-int |
| chr20 | 59440546-59440796 | + | ERV3-16A3_I-int |
| chr21 | 23279349-23279497 | − | ERV3-16A3_I-int |
| chr2 | 23312373-23312570 | + | ERV3-16A3_I-int |
| chr3 | 187376395-187378966 | − | ERV3-16A3_I-int |
| chr3 | 82035185-82035935 | + | ERV3-16A3_I-int |
| chr4 | 156708349-156709328 | − | ERV3-16A3_I-int |
| chr4 | 28993802-28994569 | + | ERV3-16A3_I-int |
| chr5 | 129441007-129441828 | + | ERV3-16A3_I-int |
| chr5 | 77121989-77122683 | − | ERV3-16A3_I-int |
| chr6 | 160321590-160322116 | + | ERV3-16A3_I-int |
| chr6 | 2596055-2596633 | + | ERV3-16A3_I-int |
| chr7 | 127543174-127544074 | + | ERV3-16A3_I-int |
| chr8 | 139375648-139376424 | − | ERV3-16A3_I-int |
| chrX | 137939107-137939819 | + | ERV3-16A3_I-int |
| chrX | 46165524-46165753 | − | ERV3-16A3_I-int |
| chrX | 51137523-51139979 | + | ERV3-16A3_I-int |
| chr10 | 107358977-107359565 | + | ERV3-16A3_I-int |
| chr10 | 107363181-107363824 | + | ERV3-16A3_I-int |
| chr10 | 107641451-107642424 | − | ERV3-16A3_I-int |
| chr10 | 108232775-108234185 | − | ERV3-16A3_I-int |
| chr10 | 115524486-115526688 | − | ERV3-16A3_I-int |
| chr10 | 17250750-17251246 | − | ERV3-16A3_I-int |
| chr10 | 33581929-33582939 | − | ERV3-16A3_I-int |
| chr10 | 33583246-33584055 | − | ERV3-16A3_I-int |
| chr10 | 35275775-35276377 | − | ERV3-16A3_I-int |
| chr10 | 5677259-5677831 | − | ERV3-16A3_I-int |
| chr10 | 78151448-78152495 | − | ERV3-16A3_I-int |
| chr10 | 7815733-7816400 | + | ERV3-16A3_I-int |
| chr10 | 78822740-78825167 | − | ERV3-16A3_I-int |
| chr10 | 78826609-78827196 | − | ERV3-16A3_I-int |
| chr11 | 101180310-101182853 | + | ERV3-16A3_I-int |
| chr11 | 107954401-107954632 | − | ERV3-16A3_I-int |
| chr11 | 131067555-131069044 | + | ERV3-16A3_I-int |
| chr11 | 132587613-132588483 | + | ERV3-16A3_I-int |
| chr11 | 134700215-134700340 | + | ERV3-16A3_I-int |
| chr11 | 4899280-4901110 | − | ERV3-16A3_I-int |
| chr1 | 154306075-154306867 | − | ERV3-16A3_I-int |
| chr11 | 72543284-72545232 | − | ERV3-16A3_I-int |
| chr1 | 182494569-182495967 | − | ERV3-16A3_I-int |
| chr11 | 90706191-90706637 | − | ERV3-16A3_I-int |
| chr11 | 99321460-99322004 | − | ERV3-16A3_I-int |
| chr1 | 206976326-206977103 | − | ERV3-16A3_I-int |
| chr1 | 209447143-209447563 | − | ERV3-16A3_I-int |
| chr12 | 100366076-100367203 | − | ERV3-16A3_I-int |
| chr12 | 119330223-119331111 | + | ERV3-16A3_I-int |
| chr12 | 133066885-133068132 | + | ERV3-16A3_I-int |
| chr1 | 215248823-215249753 | − | ERV3-16A3_I-int |
| chr12 | 25437598-25439433 | − | ERV3-16A3_I-int |
| chr12 | 25441160-25442353 | − | ERV3-16A3_I-int |
| chr1 | 227354151-227355425 | − | ERV3-16A3_I-int |
| chr1 | 229143909-229145324 | + | ERV3-16A3_I-int |
| chr12 | 30147957-30149171 | + | ERV3-16A3_I-int |
| chr12 | 30149834-30150341 | + | ERV3-16A3_I-int |
| chr12 | 89784137-89784577 | + | ERV3-16A3_I-int |
| chr1 | 29474359-29474542 | − | ERV3-16A3_I-int |
| chr13 | 32958144-32959234 | − | ERV3-16A3_I-int |
| chr13 | 36242858-36243066 | + | ERV3-16A3_I-int |
| chr13 | 36266240-36266714 | − | ERV3-16A3_I-int |
| chr13 | 44324541-44326166 | − | ERV3-16A3_I-int |
| chr13 | 56737298-56737581 | + | ERV3-16A3_I-int |
| chr13 | 62984788-62985300 | − | ERV3-16A3_I-int |
| chr13 | 76589678-76590803 | − | ERV3-16A3_I-int |
| chr13 | 77338311-77339076 | + | ERV3-16A3_I-int |
| chr1 | 38155702-38159588 | + | ERV3-16A3_I-int |
| chr14 | 62424172-62424507 | − | ERV3-16A3_I-int |
| chr14 | 62424578-62425598 | − | ERV3-16A3_I-int |
| chr14 | 84760135-84760405 | + | ERV3-16A3_I-int |
| chr15 | 37150328-37150548 | + | ERV3-16A3_I-int |
| chr15 | 37153544-37153703 | + | ERV3-16A3_I-int |
| chr15 | 39129576-39130619 | + | ERV3-16A3_I-int |
| chr15 | 40319653-40320277 | − | ERV3-16A3_I-int |
| chr1 | 54101969-54103225 | + | ERV3-16A3_I-int |
| chr15 | 45973789-45975068 | + | ERV3-16A3_I-int |
| chr15 | 76315539-76317369 | − | ERV3-16A3_I-int |
| chr15 | 80656177-80656491 | − | ERV3-16A3_I-int |
| chr1 | 60535594-60536372 | + | ERV3-16A3_I-int |
| chr1 | 64730825-64731541 | − | ERV3-16A3_I-int |
| chr16 | 49101044-49104297 | − | ERV3-16A3_I-int |
| chr16 | 50897674-50898985 | + | ERV3-16A3_I-int |
| chr16 | 76562782-76564017 | − | ERV3-16A3_I-int |
| chr17 | 34298454-34299385 | − | ERV3-16A3_I-int |
| chr17 | 41172933-41173293 | + | ERV3-16A3_I-int |
| chr17 | 45882534-45883810 | − | ERV3-16A3_I-int |
| chr17 | 45885474-45885916 | − | ERV3-16A3_I-int |
| chr17 | 62181213-62181704 | − | ERV3-16A3_I-int |
| chr17 | 62187773-62187915 | − | ERV3-16A3_I-int |
| chr17 | 71342433-71343670 | + | ERV3-16A3_I-int |
| chr18 | 10147467-10148117 | − | ERV3-16A3_I-int |
| chr18 | 39901624-39902259 | + | ERV3-16A3_I-int |
| chr18 | 46275422-46276210 | + | ERV3-16A3_I-int |
| chr18 | 68737556-68738993 | − | ERV3-16A3_I-int |
| chr1 | 93027536-93028194 | − | ERV3-16A3_I-int |
| chr19 | 31898817-31900435 | − | ERV3-16A3_I-int |
| chr19 | 33785393-33785667 | + | ERV3-16A3_I-int |
| chr20 | 39625008-39625705 | + | ERV3-16A3_I-int |
| chr2 | 105129457-105130953 | − | ERV3-16A3_I-int |
| chr2 | 112603915-112604331 | + | ERV3-16A3_I-int |
| chr2 | 122707786-122708282 | − | ERV3-16A3_I-int |
| chr21 | 27450915-27451249 | − | ERV3-16A3_I-int |
| chr2 | 131739184-131739547 | − | ERV3-16A3_I-int |
| chr2 | 132473051-132475755 | − | ERV3-16A3_I-int |
| chr21 | 33289297-33289640 | − | ERV3-16A3_I-int |
| chr21 | 39572698-39573634 | − | ERV3-16A3_I-int |
| chr2 | 142579252-142580405 | + | ERV3-16A3_I-int |
| chr21 | 42599804-42600918 | − | ERV3-16A3_I-int |
| chr21 | 46094126-46094981 | − | ERV3-16A3_I-int |
| chr21 | 46451755-46453743 | − | ERV3-16A3_I-int |
| chr2 | 152067660-152068848 | + | ERV3-16A3_I-int |
| chr2 | 160782801-160784004 | + | ERV3-16A3_I-int |
| chr2 | 163229286-163231051 | − | ERV3-16A3_I-int |
| chr2 | 178421402-178423900 | − | ERV3-16A3_I-int |
| chr2 | 180443129-180444804 | − | ERV3-16A3_I-int |
| chr2 | 183727078-183728069 | − | ERV3-16A3_I-int |
| chr2 | 205872789-205874090 | + | ERV3-16A3_I-int |
| chr2 | 205874706-205874874 | + | ERV3-16A3_I-int |
| chr2 | 216034581-216035081 | + | ERV3-16A3_I-int |
| chr2 | 234617784-234618466 | + | ERV3-16A3_I-int |
| chr2 | 2440641-2441059 | + | ERV3-16A3_I-int |
| chr22 | 49931720-49932288 | − | ERV3-16A3_I-int |
| chr2 | 65952925-65956643 | − | ERV3-16A3_I-int |
| chr2 | 81420562-81420779 | − | ERV3-16A3_I-int |
| chr2 | 8297197-8299795 | − | ERV3-16A3_I-int |
| chr2 | 88205171-88206229 | + | ERV3-16A3_I-int |
| chr2 | 96236828-96238329 | + | ERV3-16A3_I-int |
| chr3 | 108234498-108235718 | − | ERV3-16A3_I-int |
| chr3 | 12712333-12712683 | − | ERV3-16A3_I-int |
| chr3 | 135533165-135533588 | − | ERV3-16A3_I-int |
| chr3 | 13645390-13645661 | + | ERV3-16A3_I-int |
| chr3 | 197591652-197592488 | − | ERV3-16A3_I-int |
| chr3 | 84447332-84447658 | + | ERV3-16A3_I-int |
| chr4 | 10222029-10223751 | − | ERV3-16A3_I-int |
| chr4 | 114382862-114383147 | + | ERV3-16A3_I-int |
| chr4 | 120648495-120649109 | − | ERV3-16A3_I-int |
| chr4 | 122118840-122120589 | + | ERV3-16A3_I-int |
| chr4 | 12409633-12410598 | + | ERV3-16A3_I-int |
| chr4 | 140289102-140290052 | − | ERV3-16A3_I-int |
| chr4 | 149029569-149031932 | − | ERV3-16A3_I-int |
| chr4 | 165736310-165736514 | + | ERV3-16A3_I-int |
| chr4 | 171794623-171795540 | − | ERV3-16A3_I-int |
| chr4 | 186513296-186514162 | + | ERV3-16A3_I-int |
| chr4 | 24956553-24957612 | + | ERV3-16A3_I-int |
| chr4 | 67043410-67044448 | − | ERV3-16A3_I-int |
| chr4 | 67983851-67985637 | + | ERV3-16A3_I-int |
| chr4 | 77382820-77383207 | − | ERV3-16A3_I-int |
| chr4 | 86432933-86433173 | + | ERV3-16A3_I-int |
| chr5 | 108672657-108673962 | + | ERV3-16A3_I-int |
| chr5 | 109244639-109245374 | + | ERV3-16A3_I-int |
| chr5 | 115925050-115926292 | + | ERV3-16A3_I-int |
| chr5 | 12781643-12781932 | − | ERV3-16A3_I-int |
| chr5 | 129088928-129090254 | − | ERV3-16A3_I-int |
| chr5 | 136370336-136370791 | + | ERV3-16A3_I-int |
| chr5 | 144843151-144843864 | − | ERV3-16A3_I-int |
| chr5 | 163229983-163231206 | − | ERV3-16A3_I-int |
| chr5 | 24427548-24428685 | − | ERV3-16A3_I-int |
| chr5 | 3218701-3219555 | + | ERV3-16A3_I-int |
| chr5 | 44188807-44189130 | − | ERV3-16A3_I-int |
| chr5 | 44189703-44192352 | − | ERV3-16A3_I-int |
| chr5 | 4537819-4540168 | + | ERV3-16A3_I-int |
| chr5 | 4783978-4784112 | + | ERV3-16A3_I-int |
| chr5 | 5599027-5600548 | − | ERV3-16A3_I-int |
| chr5 | 61173262-61174398 | + | ERV3-16A3_I-int |
| chr5 | 7036682-7037095 | − | ERV3-16A3_I-int |
| chr5 | 71987087-71988092 | + | ERV3-16A3_I-int |
| chr5 | 96059272-96060780 | + | ERV3-16A3_I-int |
| chr5 | 96346470-96347455 | − | ERV3-16A3_I-int |
| chr5 | 97186903-97187639 | + | ERV3-16A3_I-int |
| chr6 | 11498621-11500223 | − | ERV3-16A3_I-int |
| chr6 | 138418236-138420425 | + | ERV3-16A3_I-int |
| chr6 | 145054674-145058523 | + | ERV3-16A3_I-int |
| chr6 | 146198941-146199737 | − | ERV3-16A3_I-int |
| chr6 | 19372426-19375232 | + | ERV3-16A3_I-int |
| chr6 | 2528675-2529310 | + | ERV3-16A3_I-int |
| chr6 | 2530740-2531048 | + | ERV3-16A3_I-int |
| chr6 | 29768941-29771387 | + | ERV3-16A3_I-int |
| chr6 | 61623313-61625363 | + | ERV3-16A3_I-int |
| chr6 | 67047690-67048985 | − | ERV3-16A3_I-int |
| chr6 | 81478164-81478323 | − | ERV3-16A3_I-int |
| chr6 | 86489849-86489980 | + | ERV3-16A3_I-int |
| chr6 | 95945653-95946000 | + | ERV3-16A3_I-int |
| chr6 | 9720920-9721959 | − | ERV3-16A3_I-int |
| chr7 | 117948053-117949711 | + | ERV3-16A3_I-int |
| chr7 | 120231651-120232856 | + | ERV3-16A3_I-int |
| chr7 | 137633234-137636485 | + | ERV3-16A3_I-int |
| chr7 | 34772796-34775289 | − | ERV3-16A3_I-int |
| chr7 | 49925111-49925797 | − | ERV3-16A3_I-int |
| chr7 | 50858525-50859247 | + | ERV3-16A3_I-int |
| chr7 | 80521038-80521442 | − | ERV3-16A3_I-int |
| chr7 | 90465492-90466384 | − | ERV3-16A3_I-int |
| chr7 | 94075534-94075927 | + | ERV3-16A3_I-int |
| chr7 | 94563667-94565250 | − | ERV3-16A3_I-int |
| chr8 | 109500700-109502047 | − | ERV3-16A3_I-int |
| chr8 | 113763486-113764205 | − | ERV3-16A3_I-int |
| chr8 | 117475665-117478110 | − | ERV3-16A3_I-int |
| chr8 | 117596644-117597264 | + | ERV3-16A3_I-int |
| chr8 | 118816889-118817390 | + | ERV3-16A3_I-int |
| chr8 | 119251541-119251871 | − | ERV3-16A3_I-int |
| chr8 | 134357658-134357860 | − | ERV3-16A3_I-int |
| chr8 | 136256592-136257507 | − | ERV3-16A3_I-int |
| chr8 | 20489935-20491317 | − | ERV3-16A3_I-int |
| chr8 | 26062569-26064763 | − | ERV3-16A3_I-int |
| chr8 | 30990299-30990667 | + | ERV3-16A3_I-int |
| chr8 | 52190360-52193203 | + | ERV3-16A3_I-int |
| chr8 | 58225234-58226053 | − | ERV3-16A3_I-int |
| chr8 | 61042797-61045856 | + | ERV3-16A3_I-int |
| chr8 | 69210157-69210608 | − | ERV3-16A3_I-int |
| chr8 | 90773646-90774788 | − | ERV3-16A3_I-int |
| chr9 | 11453028-11454081 | + | ERV3-16A3_I-int |
| chr9 | 129572243-129572394 | + | ERV3-16A3_I-int |
| chr9 | 131994793-131996596 | + | ERV3-16A3_I-int |
| chr9 | 38274754-38275043 | + | ERV3-16A3_I-int |
| chr9 | 98936191-98937788 | − | ERV3-16A3_I-int |
| chrX | 111792487-111793270 | + | ERV3-16A3_I-int |
| chrX | 134255760-134257034 | − | ERV3-16A3_I-int |
| chrX | 138332689-138333486 | − | ERV3-16A3_I-int |
| chrX | 142778365-142779479 | + | ERV3-16A3_I-int |
| chrX | 153034407-153034705 | + | ERV3-16A3_I-int |
| chrX | 30910599-30913573 | + | ERV3-16A3_I-int |
| chrX | 40527256-40527943 | − | ERV3-16A3_I-int |
| chrX | 49853457-49855022 | − | ERV3-16A3_I-int |
| chr13 | 95373985-95374027 | + | ERV3-16A3_LTR |
| chr17 | 70341819-70342125 | + | ERV3-16A3_LTR |
| chr4 | 22004764-22005076 | − | ERV3-16A3_LTR |
| chr8 | 95010787-95011140 | − | ERV3-16A3_LTR |
| chr10 | 128360400-128360580 | − | ERV3-16A3_LTR |
| chr1 | 118346639-118346978 | + | ERV3-16A3_LTR |
| chr16 | 10100909-10101338 | + | ERV3-16A3_LTR |
| chr17 | 50505904-50506106 | + | ERV3-16A3_LTR |
| chr18 | 10147110-10147437 | − | ERV3-16A3_LTR |
| chr18 | 1828596-1829054 | + | ERV3-16A3_LTR |
| chr3 | 176256700-176257173 | + | ERV3-16A3_LTR |
| chr4 | 111846418-111846656 | + | ERV3-16A3_LTR |
| chr5 | 117568216-117568601 | + | ERV3-16A3_LTR |
| chr7 | 123400756-123401003 | − | ERV3-16A3_LTR |
| chr7 | 125877281-125877703 | − | ERV3-16A3_LTR |
| chr9 | 101241544-101241789 | − | ERV3-16A3_LTR |
| chr1 | 173703508-173703868 | − | HERV16-int |
| chr14 | 25659918-25660870 | − | HERV16-int |
| chr15 | 92377502-92378772 | + | HERV16-int |
| chr5 | 163232619-163233209 | − | HERV16-int |
| chr9 | 117122038-117122622 | + | HERV16-int |
| chr10 | 118577503-118578365 | + | HERV16-int |
| chr11 | 103863432-103864646 | + | HERV16-int |
| chr1 | 115246296-115247683 | − | HERV16-int |
| chr11 | 59324391-59324691 | − | HERV16-int |
| chr1 | 192492501-192493084 | + | HERV16-int |
| chr11 | 98074376-98075260 | − | HERV16-int |
| chr13 | 106616617-106617148 | − | HERV16-int |
| chr13 | 109372586-109372885 | − | HERV16-int |
| chr13 | 114135458-114135686 | + | HERV16-int |
| chr13 | 32874564-32875329 | + | HERV16-int |
| chr14 | 81574763-81575258 | + | HERV16-int |
| chr15 | 31241107-31241649 | − | HERV16-int |
| chr1 | 58990247-58991426 | − | HERV16-int |
| chr15 | 99963765-99964437 | + | HERV16-int |
| chr18 | 2624982-2625564 | + | HERV16-int |
| chr21 | 17249629-17250045 | + | HERV16-int |
| chr2 | 175355377-175356810 | + | HERV16-int |
| chr2 | 238704582-238705223 | − | HERV16-int |
| chr3 | 1081844-1082645 | + | HERV16-int |
| chr3 | 11928190-11928766 | − | HERV16-int |
| chr3 | 164191462-164192380 | + | HERV16-int |
| chr3 | 84786966-84787528 | + | HERV16-int |
| chr4 | 131163925-131164968 | + | HERV16-int |
| chr4 | 183029762-183030075 | − | HERV16-int |
| chr5 | 32680053-32680977 | + | HERV16-int |
| chr5 | 39931246-39931492 | − | HERV16-int |
| chr5 | 54767312-54768090 | + | HERV16-int |
| chr5 | 68697978-68698952 | + | HERV16-int |
| chr6 | 34490366-34490901 | − | HERV16-int |
| chr6 | 4946889-4947098 | − | HERV16-int |
| chr6 | 50245995-50246759 | + | HERV16-int |
| chr7 | 144563807-144565551 | + | HERV16-int |
| chr8 | 122654767-122655849 | + | HERV16-int |
| chr8 | 13753750-13753853 | + | HERV16-int |
| chr9 | 82525683-82526337 | + | HERV16-int |
| chrX | 133134363-133134721 | + | HERV16-int |
| chr9 | 115779728-115779815 | + | LTR16 |
| chrX | 140960541-140960897 | − | LTR16 |
| chr10 | 121618096-121618203 | + | LTR16 |
| chr10 | 23803235-23803645 | + | LTR16 |
| chr10 | 90575699-90576089 | − | LTR16 |
| chr1 | 11416240-11416574 | + | LTR16 |
| chr1 | 165384604-165385033 | − | LTR16 |
| chr11 | 74494106-74494550 | − | LTR16 |
| chr11 | 98389675-98390071 | − | LTR16 |
| chr1 | 210552427-210552763 | − | LTR16 |
| chr12 | 25904557-25904925 | + | LTR16 |
| chr12 | 71464837-71465230 | − | LTR16 |
| chr12 | 93187085-93187479 | + | LTR16 |
| chr13 | 19370412-19370758 | − | LTR16 |
| chr13 | 24474356-24474704 | + | LTR16 |
| chr13 | 32391220-32391587 | − | LTR16 |
| chr13 | 46098486-46098944 | + | LTR16 |
| chr1 | 38254349-38254719 | − | LTR16 |
| chr1 | 40847235-40847635 | − | LTR16 |
| chr14 | 79434657-79435053 | − | LTR16 |
| chr15 | 61566553-61566858 | − | LTR16 |
| chr16 | 23855616-23855944 | − | LTR16 |
| chr16 | 24060502-24060930 | + | LTR16 |
| chr17 | 65460109-65460515 | − | LTR16 |
| chr17 | 73948944-73949175 | + | LTR16 |
| chr20 | 12470614-12471031 | + | LTR16 |
| chr20 | 23847044-23847420 | + | LTR16 |
| chr20 | 23869590-23869966 | + | LTR16 |
| chr20 | 48447443-48447859 | − | LTR16 |
| chr2 | 105618023-105618444 | + | LTR16 |
| chr2 | 129297367-129297675 | − | LTR16 |
| chr2 | 13280582-13280997 | − | LTR16 |
| chr21 | 33288905-33289194 | − | LTR16 |
| chr2 | 149896679-149897052 | − | LTR16 |
| chr22 | 22721666-22721821 | + | LTR16 |
| chr2 | 228331644-228332005 | + | LTR16 |
| chr2 | 38246110-38246513 | + | LTR16 |
| chr2 | 47615899-47616266 | + | LTR16 |
| chr2 | 7434126-7434545 | + | LTR16 |
| chr2 | 80791280-80791642 | + | LTR16 |
| chr3 | 141793454-141793766 | − | LTR16 |
| chr3 | 186812563-186812766 | + | LTR16 |
| chr4 | 14535317-14535661 | − | LTR16 |
| chr4 | 32578852-32579313 | − | LTR16 |
| chr4 | 37099092-37099521 | − | LTR16 |
| chr4 | 80078113-80078551 | − | LTR16 |
| chr4 | 82227419-82227735 | − | LTR16 |
| chr4 | 87767500-87767903 | + | LTR16 |
| chr4 | 88661969-88662302 | − | LTR16 |
| chr4 | 99518987-99519369 | − | LTR16 |
| chr5 | 107810289-107810494 | − | LTR16 |
| chr5 | 154324234-154324578 | + | LTR16 |
| chr5 | 27252991-27253281 | + | LTR16 |
| chr5 | 4330834-4331116 | − | LTR16 |
| chr5 | 53335219-53335624 | + | LTR16 |
| chr6 | 117228062-117228304 | + | LTR16 |
| chr6 | 123769575-123770017 | − | LTR16 |
| chr6 | 124439889-124440243 | − | LTR16 |
| chr6 | 152922583-152923004 | − | LTR16 |
| chr6 | 34487488-34487891 | − | LTR16 |
| chr6 | 56914374-56914671 | + | LTR16 |
| chr7 | 108718747-108719149 | + | LTR16 |
| chr7 | 128286400-128286757 | + | LTR16 |
| chr7 | 141239232-141239650 | − | LTR16 |
| chr7 | 153069394-153069749 | − | LTR16 |
| chr8 | 137361169-137361566 | + | LTR16 |
| chr9 | 98667816-98668243 | + | LTR16 |
| chrX | 20000282-20000703 | − | LTR16 |
| chrX | 23211051-23211495 | − | LTR16 |
| chrX | 43870963-43871303 | − | LTR16 |
| chrX | 4903949-4904307 | − | LTR16 |
| chr20 | 22622851-22623262 | − | LTR16A |
| chr20 | 23765688-23766080 | + | LTR16A |
| chr4 | 82173459-82173903 | + | LTR16A |
| chr8 | 118399341-118399767 | + | LTR16A |
| chrX | 43770018-43770464 | + | LTR16A |
| chr10 | 11781407-11781491 | − | LTR16A |
| chr10 | 128686954-128687216 | − | LTR16A |
| chr10 | 130700863-130701238 | − | LTR16A |
| chr1 | 103899826-103900257 | − | LTR16A |
| chr11 | 116171715-116172156 | − | LTR16A |
| chr11 | 127578625-127579028 | − | LTR16A |
| chr1 | 116334973-116335299 | + | LTR16A |
| chr1 | 13868761-13868989 | − | LTR16A |
| chr1 | 192238563-192238996 | + | LTR16A |
| chr12 | 114095426-114095607 | + | LTR16A |
| chr12 | 114252690-114253053 | + | LTR16A |
| chr12 | 1169186-1169601 | + | LTR16A |
| chr12 | 122297781-122298208 | + | LTR16A |
| chr1 | 220064338-220064682 | − | LTR16A |
| chr1 | 26496945-26497334 | + | LTR16A |
| chr12 | 8216708-8217050 | − | LTR16A |
| chr1 | 28595079-28595484 | + | LTR16A |
| chr13 | 51549111-51549488 | − | LTR16A |
| chr1 | 36993709-36994002 | + | LTR16A |
| chr14 | 30995801-30996179 | + | LTR16A |
| chr14 | 94946687-94947067 | − | LTR16A |
| chr15 | 45921446-45921782 | + | LTR16A |
| chr15 | 57012513-57012679 | − | LTR16A |
| chr16 | 19986831-19987108 | + | LTR16A |
| chr1 | 62537053-62537204 | + | LTR16A |
| chr16 | 55949125-55949503 | − | LTR16A |
| chr16 | 65840415-65840748 | − | LTR16A |
| chr16 | 80340019-80340404 | − | LTR16A |
| chr16 | 87545039-87545304 | − | LTR16A |
| chr17 | 52369174-52369606 | − | LTR16A |
| chr1 | 75329157-75329341 | + | LTR16A |
| chr17 | 76668142-76668319 | + | LTR16A |
| chr18 | 2180720-2181073 | − | LTR16A |
| chr18 | 30716624-30716934 | + | LTR16A |
| chr18 | 65374942-65375153 | + | LTR16A |
| chr1 | 95559862-95560220 | − | LTR16A |
| chr20 | 13926344-13926721 | − | LTR16A |
| chr20 | 22482586-22482890 | + | LTR16A |
| chr20 | 23911299-23911520 | + | LTR16A |
| chr20 | 33896701-33897104 | + | LTR16A |
| chr20 | 42730709-42731030 | + | LTR16A |
| chr20 | 46345337-46345750 | + | LTR16A |
| chr2 | 121286774-121287119 | + | LTR16A |
| chr2 | 126936273-126936671 | − | LTR16A |
| chr21 | 38597582-38597774 | − | LTR16A |
| chr2 | 139946811-139947200 | + | LTR16A |
| chr2 | 155152057-155152396 | − | LTR16A |
| chr2 | 197441377-197441725 | − | LTR16A |
| chr2 | 198639573-198639948 | − | LTR16A |
| chr2 | 216025409-216025796 | − | LTR16A |
| chr22 | 43757699-43757852 | + | LTR16A |
| chr2 | 38483987-38484398 | + | LTR16A |
| chr3 | 119172358-119172754 | + | LTR16A |
| chr3 | 132911102-132911390 | + | LTR16A |
| chr3 | 23729611-23729960 | + | LTR16A |
| chr3 | 27564367-27564768 | + | LTR16A |
| chr3 | 45035029-45035169 | − | LTR16A |
| chr3 | 45601105-45601461 | − | LTR16A |
| chr3 | 72105354-72105800 | + | LTR16A |
| chr3 | 84521547-84521884 | + | LTR16A |
| chr4 | 132648676-132649060 | + | LTR16A |
| chr4 | 143983563-143983897 | − | LTR16A |
| chr4 | 144087225-144087280 | − | LTR16A |
| chr4 | 180355643-180355961 | − | LTR16A |
| chr4 | 188660771-188661102 | − | LTR16A |
| chr4 | 45686971-45687152 | − | LTR16A |
| chr4 | 56768323-56768688 | + | LTR16A |
| chr5 | 117836499-117836837 | − | LTR16A |
| chr5 | 57891563-57892000 | − | LTR16A |
| chr5 | 74610498-74610917 | + | LTR16A |
| chr6 | 117222732-117223086 | − | LTR16A |
| chr6 | 141853079-141853537 | − | LTR16A |
| chr6 | 145059580-145059928 | + | LTR16A |
| chr6 | 17270309-17270638 | + | LTR16A |
| chr6 | 5900119-5900536 | − | LTR16A |
| chr6 | 82091859-82092003 | + | LTR16A |
| chr7 | 7555241-7555694 | + | LTR16A |
| chr8 | 111527516-111527776 | + | LTR16A |
| chr8 | 12167655-12167997 | − | LTR16A |
| chr8 | 122140896-122141330 | − | LTR16A |
| chr8 | 12409974-12410316 | − | LTR16A |
| chr8 | 133844328-133844710 | − | LTR16A |
| chr8 | 135776589-135776936 | − | LTR16A |
| chr8 | 18276869-18277096 | − | LTR16A |
| chr8 | 7302794-7303135 | + | LTR16A |
| chr8 | 8007231-8007572 | − | LTR16A |
| chr8 | 97638103-97638527 | − | LTR16A |
| chr9 | 104828111-104828529 | + | LTR16A |
| chr9 | 78503857-78504224 | − | LTR16A |
| chrX | 120668305-120668758 | + | LTR16A |
| chrX | 38925185-38925614 | + | LTR16A |
| chrX | 39552443-39552856 | + | LTR16A |
| chrX | 44760901-44761155 | + | LTR16A |
| chr10 | 71046150-71046540 | + | LTR16A1 |
| chr11 | 64478383-64478626 | − | LTR16A1 |
| chr15 | 44811697-44811815 | + | LTR16A1 |
| chr19 | 50672926-50673315 | + | LTR16A1 |
| chr3 | 24438878-24439340 | − | LTR16A1 |
| chr7 | 16615224-16615661 | + | LTR16A1 |
| chr8 | 130500260-130500615 | + | LTR16A1 |
| chr10 | 108716475-108716721 | + | LTR16A1 |
| chr11 | 125770580-125770996 | − | LTR16A1 |
| chr11 | 75110193-75110575 | − | LTR16A1 |
| chr1 | 192917288-192917731 | + | LTR16A1 |
| chr12 | 129281767-129282118 | + | LTR16A1 |
| chr13 | 38256880-38257279 | − | LTR16A1 |
| chr1 | 36907855-36908264 | + | LTR16A1 |
| chr1 | 55263682-55264000 | − | LTR16A1 |
| chr16 | 47986305-47986750 | − | LTR16A1 |
| chr1 | 75212849-75213228 | + | LTR16A1 |
| chr18 | 11161644-11162030 | − | LTR16A1 |
| chr18 | 48183636-48184034 | + | LTR16A1 |
| chr20 | 10884273-10884703 | − | LTR16A1 |
| chr20 | 2729809-2730124 | − | LTR16A1 |
| chr20 | 42113355-42113805 | + | LTR16A1 |
| chr20 | 43012669-43013007 | + | LTR16A1 |
| chr20 | 44263884-44264263 | − | LTR16A1 |
| chr20 | 4635913-4636276 | + | LTR16A1 |
| chr20 | 6180215-6180651 | + | LTR16A1 |
| chr20 | 61812137-61812491 | − | LTR16A1 |
| chr20 | 6564459-6564869 | + | LTR16A1 |
| chr20 | 9443362-9443792 | + | LTR16A1 |
| chr2 | 211017128-211017554 | − | LTR16A1 |
| chr2 | 237632247-237632450 | + | LTR16A1 |
| chr2 | 58652264-58652506 | − | LTR16A1 |
| chr2 | 6367544-6367892 | + | LTR16A1 |
| chr3 | 124259006-124259432 | − | LTR16A1 |
| chr3 | 23174537-23174935 | + | LTR16A1 |
| chr3 | 28841539-28841899 | + | LTR16A1 |
| chr3 | 38719817-38720146 | + | LTR16A1 |
| chr3 | 39757177-39757638 | − | LTR16A1 |
| chr3 | 39801114-39801534 | + | LTR16A1 |
| chr3 | 64146027-64146373 | + | LTR16A1 |
| chr3 | 78190848-78191300 | − | LTR16A1 |
| chr4 | 25306651-25306806 | + | LTR16A1 |
| chr4 | 48930611-48930992 | − | LTR16A1 |
| chr4 | 74314040-74314503 | − | LTR16A1 |
| chr5 | 10794852-10795067 | − | LTR16A1 |
| chr5 | 126675426-126675766 | + | LTR16A1 |
| chr5 | 64646110-64646567 | − | LTR16A1 |
| chr6 | 37570857-37571185 | − | LTR16A1 |
| chr6 | 84393203-84393642 | − | LTR16A1 |
| chrX | 34051648-34052124 | + | LTR16A1 |
| chrX | 39628836-39629289 | − | LTR16A1 |
| chrX | 43372721-43373186 | + | LTR16A1 |
| chr18 | 75572724-75573230 | + | LTR16A2 |
| chr2 | 209340627-209340857 | − | LTR16A2 |
| chr5 | 29716225-29716684 | + | LTR16A2 |
| chr7 | 36074199-36074665 | − | LTR16A2 |
| chrY | 15705936-15706065 | − | LTR16A2 |
| chr10 | 125545767-125546151 | − | LTR16A2 |
| chr10 | 80750621-80750989 | + | LTR16A2 |
| chr11 | 44793216-44793394 | − | LTR16A2 |
| chr12 | 51531320-51531751 | + | LTR16A2 |
| chr12 | 73103802-73103943 | + | LTR16A2 |
| chr14 | 53616866-53617253 | − | LTR16A2 |
| chr17 | 15351478-15351959 | + | LTR16A2 |
| chr17 | 15514908-15515392 | + | LTR16A2 |
| chr17 | 6166739-6167003 | + | LTR16A2 |
| chr18 | 14411640-14411949 | + | LTR16A2 |
| chr18 | 29012490-29012926 | − | LTR16A2 |
| chr18 | 41669692-41669985 | + | LTR16A2 |
| chr18 | 64673605-64674038 | − | LTR16A2 |
| chr18 | 8492728-8492973 | − | LTR16A2 |
| chr19 | 28437452-28437799 | − | LTR16A2 |
| chr20 | 24565983-24566156 | − | LTR16A2 |
| chr20 | 43215280-43215693 | + | LTR16A2 |
| chr21 | 13744330-13744632 | − | LTR16A2 |
| chr2 | 137894580-137894880 | − | LTR16A2 |
| chr21 | 6879281-6879583 | − | LTR16A2 |
| chr2 | 191645899-191646356 | − | LTR16A2 |
| chr2 | 241186920-241187271 | + | LTR16A2 |
| chr2 | 56392564-56393009 | − | LTR16A2 |
| chr3 | 171544027-171544492 | − | LTR16A2 |
| chr3 | 23928564-23928669 | − | LTR16A2 |
| chr3 | 29331163-29331597 | + | LTR16A2 |
| chr3 | 29556644-29557137 | + | LTR16A2 |
| chr3 | 41111118-41111552 | + | LTR16A2 |
| chr4 | 93568858-93569352 | + | LTR16A2 |
| chr5 | 45018684-45019041 | + | LTR16A2 |
| chr6 | 15684636-15684923 | − | LTR16A2 |
| chr6 | 24378207-24378672 | + | LTR16A2 |
| chr7 | 97669943-97670364 | − | LTR16A2 |
| chr8 | 102997186-102997388 | + | LTR16A2 |
| chr8 | 49516414-49516868 | − | LTR16A2 |
| chr10 | 115903905-115904362 | − | LTR16B |
| chr1 | 236724401-236724819 | − | LTR16B |
| chr1 | 49631669-49632050 | − | LTR16B |
| chr17 | 67457869-67458277 | + | LTR16B |
| chr18 | 21184484-21184900 | − | LTR16B |
| chr18 | 25415115-25415490 | + | LTR16B |
| chr18 | 4761669-4762071 | + | LTR16B |
| chr3 | 131171888-131172294 | − | LTR16B |
| chr3 | 189620536-189620816 | − | LTR16B |
| chr4 | 143503250-143503644 | + | LTR16B |
| chr8 | 21659393-21659808 | + | LTR16B |
| chr8 | 33959406-33959707 | + | LTR16B |
| chr8 | 8743540-8743817 | + | LTR16B |
| chr9 | 102655364-102655560 | − | LTR16B |
| chr9 | 40800829-40801235 | + | LTR16B |
| chr9 | 64921618-64922024 | + | LTR16B |
| chr10 | 10144949-10145298 | + | LTR16B1 |
| chr10 | 55546805-55547248 | − | LTR16B1 |
| chr11 | 102012288-102012731 | + | LTR16B1 |
| chr11 | 73957783-73958194 | − | LTR16B1 |
| chr11 | 75536555-75536700 | − | LTR16B1 |
| chr12 | 21554274-21554498 | + | LTR16B1 |
| chr13 | 92788697-92788891 | − | LTR16B1 |
| chr14 | 37729346-37729735 | + | LTR16B1 |
| chr14 | 58992781-58993160 | + | LTR16B1 |
| chr14 | 76091278-76091628 | + | LTR16B1 |
| chr15 | 40477697-40478121 | − | LTR16B1 |
| chr1 | 58311449-58311842 | + | LTR16B1 |
| chr16 | 61807578-61807903 | + | LTR16B1 |
| chr17 | 8706173-8706344 | − | LTR16B1 |
| chr21 | 40014477-40014815 | + | LTR16B1 |
| chr2 | 200181030-200181452 | + | LTR16B1 |
| chr2 | 200277999-200278468 | + | LTR16B1 |
| chr2 | 30129650-30129954 | + | LTR16B1 |
| chr3 | 114936818-114937031 | − | LTR16B1 |
| chr3 | 159646202-159646646 | + | LTR16B1 |
| chr3 | 190300097-190300448 | + | LTR16B1 |
| chr4 | 14964925-14965387 | − | LTR16B1 |
| chr4 | 178196800-178197186 | + | LTR16B1 |
| chr4 | 36916472-36916778 | + | LTR16B1 |
| chr4 | 76465248-76465734 | + | LTR16B1 |
| chr4 | 89797096-89797530 | + | LTR16B1 |
| chr5 | 54279858-54280296 | + | LTR16B1 |
| chr6 | 4350013-4350415 | + | LTR16B1 |
| chr7 | 46901304-46901745 | + | LTR16B1 |
| chr7 | 94343210-94343642 | + | LTR16B1 |
| chr8 | 53514548-53514992 | − | LTR16B1 |
| chr9 | 22909359-22909864 | − | LTR16B1 |
| chr9 | 2849984-2850454 | − | LTR16B1 |
| chrX | 135573173-135573639 | − | LTR16B1 |
| chr10 | 29382682-29382888 | + | LTR16B2 |
| chr10 | 30594866-30595072 | − | LTR16B2 |
| chr6 | 33941515-33941930 | − | LTR16B2 |
| chr10 | 117711378-117711828 | + | LTR16B2 |
| chr10 | 29382617-29382670 | + | LTR16B2 |
| chr11 | 130593726-130594082 | − | LTR16B2 |
| chr1 | 115893067-115893514 | + | LTR16B2 |
| chr14 | 82130383-82130460 | − | LTR16B2 |
| chr1 | 59060856-59061293 | + | LTR16B2 |
| chr1 | 72588217-72588729 | − | LTR16B2 |
| chr18 | 71975752-71976077 | + | LTR16B2 |
| chr2 | 208638483-208638619 | − | LTR16B2 |
| chr2 | 237915557-237915889 | − | LTR16B2 |
| chr2 | 58640053-58640399 | + | LTR16B2 |
| chr3 | 6780468-6780872 | + | LTR16B2 |
| chr4 | 155944590-155944944 | + | LTR16B2 |
| chr4 | 188435484-188435849 | + | LTR16B2 |
| chr5 | 121853852-121854253 | − | LTR16B2 |
| chr5 | 59684624-59685050 | + | LTR16B2 |
| chr7 | 8988274-8988618 | − | LTR16B2 |
| chr8 | 102576844-102577258 | − | LTR16B2 |
| chr8 | 109804685-109805143 | − | LTR16B2 |
| chr9 | 116566635-116567028 | + | LTR16B2 |
| chrX | 20933199-20933610 | − | LTR16B2 |
| chrX | 40881858-40882103 | − | LTR16B2 |
| chrX | 62872691-62872956 | + | LTR16B2 |
| chr3 | 45818665-45818847 | + | LTR16C |
| chr3 | 52176864-52177224 | − | LTR16C |
| chr11 | 43980991-43981373 | − | LTR16C |
| chr12 | 109645934-109646336 | − | LTR16C |
| chr12 | 73337596-73338064 | − | LTR16C |
| chr13 | 39177461-39177864 | − | LTR16C |
| chr16 | 56368001-56368344 | + | LTR16C |
| chr16 | 86143862-86144298 | + | LTR16C |
| chr17 | 17025316-17025470 | − | LTR16C |
| chr17 | 44300032-44300328 | − | LTR16C |
| chr19 | 13364940-13365137 | + | LTR16C |
| chr20 | 17488600-17488847 | + | LTR16C |
| chr20 | 21987056-21987278 | + | LTR16C |
| chr20 | 25176236-25176293 | + | LTR16C |
| chr2 | 132753903-132754036 | + | LTR16C |
| chr22 | 49677315-49677680 | − | LTR16C |
| chr3 | 150223253-150223544 | + | LTR16C |
| chr3 | 43948571-43948731 | − | LTR16C |
| chr3 | 46854840-46855131 | + | LTR16C |
| chr4 | 146853884-146854252 | + | LTR16C |
| chr5 | 154058798-154059277 | − | LTR16C |
| chr5 | 57427668-57427951 | − | LTR16C |
| chr6 | 34046388-34046657 | − | LTR16C |
| chr6 | 41952229-41952712 | − | LTR16C |
| chr6 | 61989751-61990188 | + | LTR16C |
| chr7 | 127256661-127257120 | − | LTR16C |
| chr7 | 134370532-134371036 | − | LTR16C |
| chr7 | 91561116-91561215 | − | LTR16C |
| chr8 | 130411281-130411612 | + | LTR16C |
| chr8 | 133818132-133818271 | + | LTR16C |
| chr8 | 134739808-134740177 | + | LTR16C |
| chr9 | 101200063-101200454 | + | LTR16C |
| chr9 | 38150084-38150465 | + | LTR16C |
| chr9 | 82120052-82120126 | − | LTR16C |
| chrX | 13428591-13429025 | + | LTR16C |
| chr10 | 10092607-10092902 | − | LTR16C |
| chr10 | 103737416-103737746 | − | LTR16C |
| chr10 | 125377398-125377629 | − | LTR16C |
| chr10 | 15027150-15027409 | + | LTR16C |
| chr10 | 48554478-48554888 | + | LTR16C |
| chr10 | 54065247-54065481 | + | LTR16C |
| chr10 | 71076909-71077362 | − | LTR16C |
| chr10 | 71378388-71378544 | + | LTR16C |
| chr10 | 71378735-71378948 | + | LTR16C |
| chr10 | 73891445-73891735 | − | LTR16C |
| chr10 | 78172817-78173295 | + | LTR16C |
| chr1 | 108328754-108329153 | + | LTR16C |
| chr1 | 110730432-110730807 | + | LTR16C |
| chr11 | 1654683-1654800 | + | LTR16C |
| chr11 | 22040557-22040760 | + | LTR16C |
| chr11 | 30720502-30720946 | + | LTR16C |
| chr11 | 61487351-61487770 | − | LTR16C |
| chr1 | 18405020-18405277 | + | LTR16C |
| chr1 | 18499017-18499144 | − | LTR16C |
| chr1 | 188221718-188222158 | − | LTR16C |
| chr11 | 91133279-91133530 | + | LTR16C |
| chr12 | 110080550-110080661 | + | LTR16C |
| chr12 | 110766798-110766964 | + | LTR16C |
| chr12 | 111013326-111013718 | + | LTR16C |
| chr12 | 114957098-114957254 | − | LTR16C |
| chr12 | 118723533-118723768 | − | LTR16C |
| chr12 | 127235924-127236151 | + | LTR16C |
| chr12 | 130623620-130624070 | + | LTR16C |
| chr1 | 33894271-33894739 | + | LTR16C |
| chr13 | 50708468-50708948 | − | LTR16C |
| chr1 | 36688178-36688300 | − | LTR16C |
| chr13 | 74258801-74259226 | − | LTR16C |
| chr14 | 38032733-38033120 | + | LTR16C |
| chr14 | 41427054-41427350 | + | LTR16C |
| chr14 | 50423773-50424148 | + | LTR16C |
| chr14 | 52355616-52356054 | + | LTR16C |
| chr14 | 65227548-65227665 | + | LTR16C |
| chr14 | 68855402-68855645 | − | LTR16C |
| chr14 | 69866977-69867391 | − | LTR16C |
| chr14 | 82022540-82022928 | − | LTR16C |
| chr1 | 48512608-48513071 | + | LTR16C |
| chr1 | 48699417-48699876 | + | LTR16C |
| chr14 | 92202975-92203313 | − | LTR16C |
| chr14 | 92214313-92214591 | + | LTR16C |
| chr14 | 92558529-92558870 | − | LTR16C |
| chr1 | 50372782-50373229 | + | LTR16C |
| chr15 | 33392341-33392780 | − | LTR16C |
| chr15 | 61518583-61519017 | + | LTR16C |
| chr1 | 5768693-5768829 | + | LTR16C |
| chr15 | 79607125-79607522 | + | LTR16C |
| chr1 | 60098986-60099321 | + | LTR16C |
| chr16 | 13007934-13008411 | + | LTR16C |
| chr16 | 18059544-18059981 | + | LTR16C |
| chr16 | 52823419-52823561 | + | LTR16C |
| chr16 | 55743073-55743538 | − | LTR16C |
| chr16 | 59027770-59028076 | − | LTR16C |
| chr16 | 63673646-63673947 | − | LTR16C |
| chr16 | 67495874-67496180 | − | LTR16C |
| chr16 | 77774411-77774806 | + | LTR16C |
| chr16 | 77976996-77977453 | + | LTR16C |
| chr16 | 77983244-77983405 | − | LTR16C |
| chr16 | 81170245-81170609 | − | LTR16C |
| chr16 | 82718001-82718297 | − | LTR16C |
| chr1 | 69122639-69123098 | − | LTR16C |
| chr1 | 71202711-71203153 | − | LTR16C |
| chr17 | 27921321-27921692 | + | LTR16C |
| chr17 | 43684835-43685172 | − | LTR16C |
| chr17 | 45766219-45766427 | + | LTR16C |
| chr17 | 45836557-45836815 | − | LTR16C |
| chr1 | 76858199-76858653 | + | LTR16C |
| chr1 | 79140771-79141192 | − | LTR16C |
| chr18 | 31363886-31364336 | − | LTR16C |
| chr18 | 56434184-56434436 | + | LTR16C |
| chr18 | 7723617-7723911 | + | LTR16C |
| chr19 | 13442130-13442455 | − | LTR16C |
| chr19 | 13643514-13643968 | − | LTR16C |
| chr19 | 14615301-14615428 | + | LTR16C |
| chr19 | 15142605-15143000 | − | LTR16C |
| chr19 | 15467524-15467785 | + | LTR16C |
| chr19 | 28250818-28251200 | − | LTR16C |
| chr19 | 28396989-28397079 | − | LTR16C |
| chr19 | 29581470-29581793 | − | LTR16C |
| chr19 | 6446407-6446729 | + | LTR16C |
| chr19 | 6886493-6886893 | + | LTR16C |
| chr20 | 10078536-10078741 | + | LTR16C |
| chr20 | 12286264-12286669 | − | LTR16C |
| chr20 | 12646739-12647103 | − | LTR16C |
| chr20 | 12837695-12837815 | − | LTR16C |
| chr20 | 13836526-13836987 | − | LTR16C |
| chr20 | 15491413-15491888 | − | LTR16C |
| chr20 | 17237650-17238120 | − | LTR16C |
| chr20 | 17480584-17481053 | − | LTR16C |
| chr20 | 17493222-17493624 | + | LTR16C |
| chr20 | 17882986-17883138 | + | LTR16C |
| chr20 | 18985190-18985597 | − | LTR16C |
| chr20 | 19317879-19318324 | + | LTR16C |
| chr20 | 19511138-19511262 | + | LTR16C |
| chr20 | 20102292-20102483 | + | LTR16C |
| chr20 | 20826817-20827257 | − | LTR16C |
| chr20 | 21113122-21113434 | + | LTR16C |
| chr20 | 21809300-21809425 | − | LTR16C |
| chr20 | 21896294-21896586 | + | LTR16C |
| chr20 | 22684815-22685087 | − | LTR16C |
| chr20 | 24152058-24152218 | + | LTR16C |
| chr20 | 25178950-25179017 | + | LTR16C |
| chr20 | 25954193-25954332 | + | LTR16C |
| chr20 | 2762478-2762953 | − | LTR16C |
| chr20 | 33288028-33288215 | − | LTR16C |
| chr20 | 33703522-33703958 | − | LTR16C |
| chr20 | 38211910-38212032 | + | LTR16C |
| chr20 | 38655718-38656011 | − | LTR16C |
| chr20 | 38657068-38657222 | − | LTR16C |
| chr20 | 39293210-39293704 | − | LTR16C |
| chr20 | 39339997-39340402 | + | LTR16C |
| chr20 | 4068041-4068472 | − | LTR16C |
| chr20 | 40861806-40862194 | + | LTR16C |
| chr20 | 4303993-4304316 | − | LTR16C |
| chr20 | 43158999-43159268 | − | LTR16C |
| chr20 | 45540319-45540509 | + | LTR16C |
| chr20 | 46614221-46614548 | − | LTR16C |
| chr20 | 4827920-4828180 | − | LTR16C |
| chr20 | 48552635-48552957 | + | LTR16C |
| chr20 | 48758012-48758183 | − | LTR16C |
| chr20 | 48912350-48912799 | + | LTR16C |
| chr20 | 52337650-52338135 | − | LTR16C |
| chr20 | 53497212-53497701 | − | LTR16C |
| chr20 | 6133567-6133935 | + | LTR16C |
| chr20 | 6239758-6240147 | + | LTR16C |
| chr20 | 9441423-9441740 | − | LTR16C |
| chr20 | 9772830-9772978 | + | LTR16C |
| chr20 | 9822735-9822964 | + | LTR16C |
| chr2 | 105953750-105954169 | − | LTR16C |
| chr21 | 14677987-14678439 | − | LTR16C |
| chr2 | 12064538-12064706 | + | LTR16C |
| chr2 | 120713994-120714328 | − | LTR16C |
| chr2 | 120892577-120892930 | + | LTR16C |
| chr2 | 126858331-126858743 | + | LTR16C |
| chr21 | 38727862-38728240 | + | LTR16C |
| chr2 | 146027192-146027413 | − | LTR16C |
| chr2 | 203810151-203810375 | − | LTR16C |
| chr2 | 206488425-206488771 | − | LTR16C |
| chr22 | 25002411-25002533 | − | LTR16C |
| chr22 | 26155127-26155562 | + | LTR16C |
| chr22 | 26891321-26891617 | − | LTR16C |
| chr22 | 27308618-27308955 | + | LTR16C |
| chr22 | 27834798-27835133 | − | LTR16C |
| chr22 | 30371018-30371286 | − | LTR16C |
| chr2 | 237182764-237183105 | − | LTR16C |
| chr2 | 240426156-240426337 | − | LTR16C |
| chr2 | 30377282-30377755 | + | LTR16C |
| chr2 | 62227785-62228015 | − | LTR16C |
| chr2 | 70771784-70772105 | + | LTR16C |
| chr2 | 72688228-72688529 | − | LTR16C |
| chr2 | 89042747-89042952 | + | LTR16C |
| chr2 | 89048845-89048954 | + | LTR16C |
| chr2 | 89279670-89279958 | − | LTR16C |
| chr2 | 89902588-89902876 | + | LTR16C |
| chr2 | 90080019-90080097 | − | LTR16C |
| chr2 | 90151637-90151719 | − | LTR16C |
| chr2 | 90157606-90157811 | − | LTR16C |
| chr2 | 90207262-90207344 | − | LTR16C |
| chr3 | 120625228-120625602 | − | LTR16C |
| chr3 | 132193822-132194274 | − | LTR16C |
| chr3 | 22114432-22114728 | − | LTR16C |
| chr3 | 26716556-26717011 | + | LTR16C |
| chr3 | 30400475-30400859 | + | LTR16C |
| chr3 | 3862536-3862779 | − | LTR16C |
| chr3 | 39391107-39391387 | + | LTR16C |
| chr3 | 44073186-44073461 | − | LTR16C |
| chr3 | 44166723-44166940 | + | LTR16C |
| chr3 | 55230790-55231017 | − | LTR16C |
| chr3 | 63704108-63704577 | − | LTR16C |
| chr3 | 67672188-67672634 | + | LTR16C |
| chr3 | 72102225-72102658 | − | LTR16C |
| chr3 | 72232677-72232857 | + | LTR16C |
| chr3 | 74748360-74748796 | − | LTR16C |
| chr3 | 8713921-8714370 | − | LTR16C |
| chr4 | 117957695-117958180 | + | LTR16C |
| chr4 | 144818495-144818934 | + | LTR16C |
| chr4 | 27223481-27223803 | − | LTR16C |
| chr4 | 52921810-52922140 | + | LTR16C |
| chr4 | 53946641-53946926 | − | LTR16C |
| chr4 | 76448474-76448766 | + | LTR16C |
| chr4 | 78621460-78621836 | + | LTR16C |
| chr4 | 8835980-8836352 | + | LTR16C |
| chr5 | 138722858-138722951 | + | LTR16C |
| chr5 | 154372454-154372712 | − | LTR16C |
| chr5 | 159860073-159860402 | − | LTR16C |
| chr5 | 5890658-5890876 | + | LTR16C |
| chr5 | 66319347-66319528 | − | LTR16C |
| chr6 | 130111085-130111488 | − | LTR16C |
| chr6 | 135877657-135878135 | − | LTR16C |
| chr6 | 156057914-156058311 | + | LTR16C |
| chr6 | 36843167-36843557 | − | LTR16C |
| chr6 | 37532738-37533181 | − | LTR16C |
| chr6 | 37709625-37709987 | + | LTR16C |
| chr6 | 40787810-40788095 | − | LTR16C |
| chr6 | 80508689-80509019 | − | LTR16C |
| chr6 | 90320843-90320969 | + | LTR16C |
| chr7 | 107884435-107884738 | + | LTR16C |
| chr7 | 148276195-148276635 | − | LTR16C |
| chr7 | 70216422-70216877 | + | LTR16C |
| chr7 | 82057990-82058328 | + | LTR16C |
| chr8 | 101014692-101014791 | − | LTR16C |
| chr8 | 125777585-125777979 | + | LTR16C |
| chr8 | 13245474-13245626 | + | LTR16C |
| chr8 | 138673798-138674169 | + | LTR16C |
| chr8 | 139302271-139302552 | − | LTR16C |
| chr8 | 141999208-141999630 | + | LTR16C |
| chr8 | 142147902-142148019 | − | LTR16C |
| chr8 | 36538691-36539124 | + | LTR16C |
| chr8 | 74491097-74491503 | + | LTR16C |
| chr9 | 106050757-106051008 | + | LTR16C |
| chr9 | 11993578-11994007 | − | LTR16C |
| chr9 | 124206218-124206683 | − | LTR16C |
| chr9 | 3372035-3372487 | + | LTR16C |
| chr9 | 61705142-61705279 | − | LTR16C |
| chr9 | 78081732-78081992 | − | LTR16C |
| chr9 | 85272991-85273471 | − | LTR16C |
| chr9 | 87039910-87040187 | − | LTR16C |
| chr9 | 90772221-90772556 | − | LTR16C |
| chr9 | 98642991-98643225 | − | LTR16C |
| chrX | 11536133-11536572 | + | LTR16C |
| chrX | 12679677-12679971 | + | LTR16C |
| chrX | 12691605-12691904 | − | LTR16C |
| chrX | 135124160-135124555 | − | LTR16C |
| chrX | 135247987-135248382 | + | LTR16C |
| chrX | 15406257-15406660 | − | LTR16C |
| chrX | 15565689-15566045 | + | LTR16C |
| chrX | 17562110-17562576 | − | LTR16C |
| chrX | 17975798-17976088 | + | LTR16C |
| chrX | 1905974-1906275 | − | LTR16C |
| chrX | 20015605-20015912 | + | LTR16C |
| chrX | 20490322-20490504 | + | LTR16C |
| chrX | 20892630-20892997 | + | LTR16C |
| chrX | 21699540-21699834 | + | LTR16C |
| chrX | 22137777-22138187 | + | LTR16C |
| chrX | 22487103-22487498 | + | LTR16C |
| chrX | 22846313-22846784 | + | LTR16C |
| chrX | 2597278-2597414 | − | LTR16C |
| chrX | 32967306-32967610 | − | LTR16C |
| chrX | 39049244-39049721 | + | LTR16C |
| chrX | 39984200-39984401 | − | LTR16C |
| chrX | 40480657-40480984 | − | LTR16C |
| chrX | 42195365-42195837 | − | LTR16C |
| chrY | 1905974-1906275 | − | LTR16C |
| chrY | 2597278-2597414 | − | LTR16C |
| chr12 | 16823924-16824466 | − | LTR16D |
| chr3 | 141633124-141633321 | + | LTR16D |
| chr4 | 132423117-132423217 | − | LTR16D |
| chr8 | 131597744-131598079 | + | LTR16D |
| chr10 | 92456144-92456218 | + | LTR16D |
| chr11 | 131070656-131071008 | + | LTR16D |
| chr12 | 89205941-89206263 | − | LTR16D |
| chr14 | 55714425-55714980 | + | LTR16D |
| chr14 | 87291690-87291871 | − | LTR16D |
| chr15 | 47685255-47685405 | − | LTR16D |
| chr15 | 66113275-66113580 | + | LTR16D |
| chr15 | 90336813-90337246 | − | LTR16D |
| chr16 | 76592024-76592521 | + | LTR16D |
| chr16 | 79555116-79555312 | + | LTR16D |
| chr18 | 2413156-2413572 | − | LTR16D |
| chr19 | 28468561-28468752 | − | LTR16D |
| chr19 | 32190144-32190543 | + | LTR16D |
| chr2 | 104803904-104804184 | + | LTR16D |
| chr21 | 17922223-17922334 | − | LTR16D |
| chr2 | 126049724-126050246 | + | LTR16D |
| chr2 | 137647599-137647955 | + | LTR16D |
| chr21 | 43155720-43155844 | + | LTR16D |
| chr21 | 6547403-6547527 | + | LTR16D |
| chr2 | 170740735-170741309 | + | LTR16D |
| chr2 | 45380505-45380906 | + | LTR16D |
| chr3 | 152096509-152096883 | + | LTR16D |
| chr3 | 18077122-18077726 | + | LTR16D |
| chr3 | 188037879-188038274 | − | LTR16D |
| chr3 | 43883108-43883483 | + | LTR16D |
| chr3 | 94092084-94092236 | + | LTR16D |
| chr4 | 167748160-167748286 | − | LTR16D |
| chr4 | 178579345-178579538 | + | LTR16D |
| chr4 | 30626438-30626886 | − | LTR16D |
| chr4 | 71326056-71326191 | + | LTR16D |
| chr5 | 112604798-112604981 | − | LTR16D |
| chr5 | 120996716-120997312 | + | LTR16D |
| chr5 | 159433501-159433860 | − | LTR16D |
| chr6 | 127011631-127012139 | + | LTR16D |
| chr6 | 53440456-53440910 | − | LTR16D |
| chr7 | 88864335-88864884 | − | LTR16D |
| chr8 | 123752280-123752624 | + | LTR16D |
| chr8 | 58967696-58967885 | + | LTR16D |
| chr8 | 96902681-96903072 | + | LTR16D |
| chrX | 43426102-43426617 | + | LTR16D |
| chrX | 7692852-7693250 | − | LTR16D |
| chrX | 43512583-43513236 | − | LTR16D1 |
| chrX | 120716666-120717087 | + | LTR16D1 |
| chr10 | 125584987-125585338 | − | LTR16D1 |
| chr14 | 55984419-55985049 | − | LTR16D1 |
| chr18 | 10388166-10388775 | − | LTR16D1 |
| chr20 | 39715162-39715593 | + | LTR16D1 |
| chr5 | 103413553-103413962 | − | LTR16D1 |
| chr16 | 61121458-61121862 | + | LTR16D2 |
| chr8 | 43233552-43233705 | + | LTR16D2 |
| chr12 | 10983395-10983951 | + | LTR16D2 |
| chr12 | 127293560-127294067 | − | LTR16D2 |
| chr12 | 12745831-12746176 | − | LTR16D2 |
| chr1 | 217737727-217737827 | − | LTR16D2 |
| chr14 | 71888449-71888711 | − | LTR16D2 |
| chr15 | 20497688-20498085 | − | LTR16D2 |
| chr15 | 22502366-22502763 | + | LTR16D2 |
| chr15 | 28597560-28597957 | + | LTR16D2 |
| chr1 | 5476624-5477153 | + | LTR16D2 |
| chr5 | 67703453-67703555 | − | LTR16D2 |
| chr7 | 125233955-125234443 | − | LTR16D2 |
| chr1 | 230009695-230010157 | − | LTR16E1 |
| chr17 | 9764550-9765043 | − | LTR16E1 |
| chr19 | 29894938-29895100 | − | LTR16E1 |
| chr20 | 58126739-58127039 | − | LTR16E1 |
| chr2 | 219324997-219325454 | − | LTR16E1 |
| chr4 | 16420861-16421163 | + | LTR16E1 |
| chr4 | 48294254-48294554 | + | LTR16E1 |
| chr8 | 130710266-130710756 | − | LTR16E1 |
| chr10 | 110373459-110373939 | + | LTR16E1 |
| chr10 | 111999864-112000371 | − | LTR16E1 |
| chr10 | 123728130-123728290 | + | LTR16E1 |
| chr10 | 124301661-124302097 | − | LTR16E1 |
| chr10 | 18282003-18282356 | − | LTR16E1 |
| chr10 | 47352095-47352207 | − | LTR16E1 |
| chr10 | 49212275-49212461 | + | LTR16E1 |
| chr10 | 53534203-53534414 | − | LTR16E1 |
| chr10 | 71378433-71378547 | + | LTR16E1 |
| chr10 | 96509377-96509658 | + | LTR16E1 |
| chr11 | 106749551-106749841 | + | LTR16E1 |
| chr1 | 111942625-111942720 | + | LTR16E1 |
| chr11 | 126828844-126829259 | − | LTR16E1 |
| chr11 | 128934686-128935187 | − | LTR16E1 |
| chr11 | 134637341-134637515 | − | LTR16E1 |
| chr11 | 5675380-5675662 | − | LTR16E1 |
| chr11 | 76639479-76639917 | − | LTR16E1 |
| chr1 | 192129777-192130256 | + | LTR16E1 |
| chr1 | 2038526-2039015 | − | LTR16E1 |
| chr12 | 113758267-113758554 | − | LTR16E1 |
| chr12 | 22872547-22872876 | − | LTR16E1 |
| chr12 | 38645085-38645587 | + | LTR16E1 |
| chr1 | 25881443-25881848 | + | LTR16E1 |
| chr1 | 29732691-29733176 | + | LTR16E1 |
| chr1 | 36668389-36668668 | − | LTR16E1 |
| chr14 | 36360839-36361354 | + | LTR16E1 |
| chr14 | 45385393-45385819 | − | LTR16E1 |
| chr14 | 76135508-76136005 | − | LTR16E1 |
| chr14 | 81639642-81639958 | + | LTR16E1 |
| chr15 | 54029757-54030235 | + | LTR16E1 |
| chr15 | 70348012-70348465 | + | LTR16E1 |
| chr15 | 88096449-88096954 | + | LTR16E1 |
| chr16 | 26951756-26952225 | − | LTR16E1 |
| chr16 | 27096323-27096683 | − | LTR16E1 |
| chr16 | 55124822-55125332 | − | LTR16E1 |
| chr16 | 82250081-82250601 | + | LTR16E1 |
| chr16 | 86944507-86944815 | − | LTR16E1 |
| chr17 | 45672224-45672286 | + | LTR16E1 |
| chr17 | 73904199-73904418 | − | LTR16E1 |
| chr18 | 23718716-23719198 | + | LTR16E1 |
| chr18 | 25641526-25641963 | − | LTR16E1 |
| chr18 | 35937998-35938101 | + | LTR16E1 |
| chr19 | 29895396-29895573 | − | LTR16E1 |
| chr19 | 30561457-30561762 | − | LTR16E1 |
| chr19 | 30725871-30726210 | + | LTR16E1 |
| chr20 | 12396972-12397464 | − | LTR16E1 |
| chr20 | 13648455-13648899 | + | LTR16E1 |
| chr20 | 14833173-14833435 | − | LTR16E1 |
| chr20 | 18845328-18845643 | − | LTR16E1 |
| chr20 | 57921015-57921208 | − | LTR16E1 |
| chr21 | 32116545-32117019 | + | LTR16E1 |
| chr21 | 39727771-39727940 | + | LTR16E1 |
| chr21 | 42041908-42042242 | + | LTR16E1 |
| chr21 | 44840733-44840920 | − | LTR16E1 |
| chr2 | 148830774-148830990 | − | LTR16E1 |
| chr2 | 182042306-182042673 | − | LTR16E1 |
| chr22 | 26713655-26714128 | − | LTR16E1 |
| chr2 | 237233425-237233978 | − | LTR16E1 |
| chr3 | 107497432-107497902 | − | LTR16E1 |
| chr3 | 107846301-107846389 | − | LTR16E1 |
| chr3 | 23994402-23994515 | − | LTR16E1 |
| chr3 | 26560473-26560952 | + | LTR16E1 |
| chr3 | 34966868-34967140 | + | LTR16E1 |
| chr3 | 38709653-38709980 | − | LTR16E1 |
| chr3 | 40901257-40901563 | − | LTR16E1 |
| chr3 | 40998412-40998780 | + | LTR16E1 |
| chr3 | 5978215-5978564 | − | LTR16E1 |
| chr3 | 63473945-63474408 | − | LTR16E1 |
| chr3 | 66608684-66608838 | + | LTR16E1 |
| chr4 | 109617971-109618540 | + | LTR16E1 |
| chr4 | 115243575-115243794 | + | LTR16E1 |
| chr4 | 133668083-133668365 | − | LTR16E1 |
| chr4 | 151833930-151834471 | − | LTR16E1 |
| chr4 | 38349868-38350289 | + | LTR16E1 |
| chr4 | 4900662-4900764 | + | LTR16E1 |
| chr4 | 5869750-5869957 | − | LTR16E1 |
| chr4 | 73864369-73864584 | − | LTR16E1 |
| chr4 | 80105618-80106077 | − | LTR16E1 |
| chr4 | 8149886-8150044 | + | LTR16E1 |
| chr4 | 96887762-96888248 | − | LTR16E1 |
| chr5 | 112644600-112644988 | + | LTR16E1 |
| chr5 | 135591466-135591927 | + | LTR16E1 |
| chr5 | 36537400-36537856 | + | LTR16E1 |
| chr5 | 51816622-51816759 | + | LTR16E1 |
| chr5 | 56704548-56704697 | − | LTR16E1 |
| chr5 | 66855496-66855879 | − | LTR16E1 |
| chr5 | 99159439-99159732 | − | LTR16E1 |
| chr6 | 10474670-10475009 | + | LTR16E1 |
| chr6 | 135581009-135581236 | + | LTR16E1 |
| chr6 | 156206423-156206682 | + | LTR16E1 |
| chr6 | 3526182-3526286 | + | LTR16E1 |
| hr6 | 41508389-41508772 | + | LTR16E1 |
| chr6 | 5875871-5876361 | − | LTR16E1 |
| chr6 | 724577-724846 | + | LTR16E1 |
| chr7 | 115037909-115038412 | − | LTR16E1 |
| chr7 | 155056298-155056600 | + | LTR16E1 |
| chr7 | 30734842-30734968 | − | LTR16E1 |
| chr8 | 135043536-135043628 | + | LTR16E1 |
| chr8 | 136873408-136873521 | + | LTR16E1 |
| chr8 | 59519972-59520136 | + | LTR16E1 |
| chr8 | 89396480-89396873 | − | LTR16E1 |
| chr9 | 11992883-11993262 | − | LTR16E1 |
| chr9 | 124139020-124139199 | + | LTR16E1 |
| chr9 | 29829301-29829751 | + | LTR16E1 |
| chr9 | 86526638-86527123 | + | LTR16E1 |
| chrX | 121562359-121562876 | − | LTR16E1 |
| chrX | 13361804-13362275 | + | LTR16E1 |
| chrX | 20350050-20350532 | − | LTR16E1 |
| chrX | 32913604-32914083 | + | LTR16E1 |
| chrX | 39253332-39253607 | − | LTR16E1 |
| chrX | 97870523-97870790 | − | LTR16E1 |
| chr21 | 26446682-26446843 | − | LTR16E2 |
| chr3 | 23443260-23443690 | − | LTR16E2 |
| chr10 | 106211158-106211349 | − | LTR16E2 |
| chr11 | 4826806-4827193 | − | LTR16E2 |
| chr1 | 24522359-24522718 | + | LTR16E2 |
| chr13 | 36146088-36146554 | − | LTR16E2 |
| chr20 | 10314831-10315112 | − | LTR16E2 |
| chr20 | 21133686-21134180 | − | LTR16E2 |
| chr2 | 104140287-104140578 | + | LTR16E2 |
| chr2 | 240148295-240148727 | − | LTR16E2 |
| chr2 | 66923827-66924357 | + | LTR16E2 |
| chr3 | 135327384-135327636 | + | LTR16E2 |
| chr5 | 111290345-111290787 | − | LTR16E2 |
| chr6 | 126302273-126302413 | − | LTR16E2 |
| chr6 | 99017360-99017832 | − | LTR16E2 |
| chr8 | 65375695-65376211 | + | LTR16E2 |
| chr9 | 85898089-85898548 | − | LTR16E2 |
| chr10 | 11625964-11626462 | − | LTR16E2 |
| chr10 | 30792697-30793064 | + | LTR16E2 |
| chr10 | 31129941-31130427 | − | LTR16E2 |
| chr10 | 43897532-43898036 | − | LTR16E2 |
| chr10 | 6600144-6600252 | − | LTR16E2 |
| chr10 | 71090661-71091225 | − | LTR16E2 |
| chr1 | 108025681-108026219 | + | LTR16E2 |
| chr1 | 112096323-112096921 | + | LTR16E2 |
| chr1 | 112102008-112102573 | + | LTR16E2 |
| chr1 | 115609146-115609421 | − | LTR16E2 |
| chr11 | 69215972-69216100 | + | LTR16E2 |
| chr11 | 72538757-72539153 | − | LTR16E2 |
| chr11 | 80245352-80245672 | + | LTR16E2 |
| chr1 | 189254718-189254904 | − | LTR16E2 |
| chr12 | 117220319-117220836 | − | LTR16E2 |
| chr12 | 118875149-118875630 | − | LTR16E2 |
| chr12 | 24664799-24664910 | + | LTR16E2 |
| chr12 | 76920748-76920862 | + | LTR16E2 |
| chr12 | 93116768-93117104 | + | LTR16E2 |
| chr13 | 106343007-106343168 | + | LTR16E2 |
| chr1 | 36835202-36835644 | + | LTR16E2 |
| chr14 | 101666388-101666686 | + | LTR16E2 |
| chr14 | 49091889-49092051 | + | LTR16E2 |
| chr14 | 54576919-54577352 | − | LTR16E2 |
| chr14 | 57816729-57817014 | + | LTR16E2 |
| chr14 | 88027604-88028053 | − | LTR16E2 |
| chr1 | 56035739-56035916 | − | LTR16E2 |
| chr15 | 66195437-66195955 | − | LTR16E2 |
| chr1 | 59412045-59412570 | + | LTR16E2 |
| chr16 | 52058883-52059169 | + | LTR16E2 |
| chr16 | 53579377-53579564 | − | LTR16E2 |
| chr16 | 73510898-73511336 | + | LTR16E2 |
| chr16 | 76194797-76195305 | − | LTR16E2 |
| chr16 | 82203074-82203548 | + | LTR16E2 |
| chr16 | 84635187-84635441 | + | LTR16E2 |
| chr16 | 9401868-9402077 | + | LTR16E2 |
| chr17 | 74057591-74058001 | − | LTR16E2 |
| chr18 | 7226398-7226632 | + | LTR16E2 |
| chr19 | 43818240-43818789 | − | LTR16E2 |
| chr20 | 10815511-10816022 | − | LTR16E2 |
| chr20 | 24148504-24148712 | + | LTR16E2 |
| chr20 | 58972873-58973333 | + | LTR16E2 |
| chr2 | 100241438-100241779 | + | LTR16E2 |
| chr2 | 114783847-114784247 | + | LTR16E2 |
| chr21 | 27472331-27472846 | − | LTR16E2 |
| chr21 | 29768318-29768703 | − | LTR16E2 |
| chr21 | 33510973-33511026 | − | LTR16E2 |
| chr2 | 136318391-136318758 | − | LTR16E2 |
| chr21 | 41973659-41974120 | + | LTR16E2 |
| chr2 | 143261717-143262228 | − | LTR16E2 |
| chr2 | 150923773-150923946 | − | LTR16E2 |
| chr2 | 182652439-182652600 | − | LTR16E2 |
| chr2 | 184848897-184849338 | − | LTR16E2 |
| chr2 | 192228504-192228995 | − | LTR16E2 |
| chr2 | 204286116-204286571 | + | LTR16E2 |
| chr2 | 213197439-213197920 | − | LTR16E2 |
| chr2 | 216944895-216945332 | + | LTR16E2 |
| chr2 | 237227644-237228087 | + | LTR16E2 |
| chr2 | 30282822-30283310 | + | LTR16E2 |
| chr2 | 65580909-65581093 | − | LTR16E2 |
| chr2 | 9064524-9064909 | + | LTR16E2 |
| chr3 | 103455226-103455664 | + | LTR16E2 |
| chr3 | 1084474-1084776 | + | LTR16E2 |
| chr3 | 170875211-170875425 | − | LTR16E2 |
| chr3 | 188609887-188610381 | − | LTR16E2 |
| chr3 | 20446805-20447314 | + | LTR16E2 |
| chr3 | 41011898-41011994 | − | LTR16E2 |
| chr3 | 411824-412336 | + | LTR16E2 |
| chr3 | 43832819-43832975 | + | LTR16E2 |
| chr3 | 44135466-44135657 | − | LTR16E2 |
| chr3 | 46285405-46285854 | + | LTR16E2 |
| chr3 | 64077458-64077637 | − | LTR16E2 |
| chr3 | 67956934-67957319 | − | LTR16E2 |
| chr3 | 74869055-74869326 | + | LTR16E2 |
| chr3 | 75110939-75111417 | + | LTR16E2 |
| chr4 | 104768333-104768715 | + | LTR16E2 |
| chr4 | 32121557-32121964 | + | LTR16E2 |
| chr4 | 75427503-75427969 | + | LTR16E2 |
| chr4 | 8171263-8171480 | + | LTR16E2 |
| chr5 | 103346950-103347299 | − | LTR16E2 |
| chr5 | 112105495-112105730 | + | LTR16E2 |
| chr5 | 115392591-115393087 | + | LTR16E2 |
| chr5 | 117452727-117453180 | + | LTR16E2 |
| chr5 | 12119719-12120200 | + | LTR16E2 |
| chr5 | 143065626-143065763 | + | LTR16E2 |
| chr5 | 43281269-43281668 | − | LTR16E2 |
| chr5 | 52065125-52065393 | − | LTR16E2 |
| chr5 | 56040137-56040417 | − | LTR16E2 |
| chr5 | 67242395-67242900 | + | LTR16E2 |
| chr5 | 67939060-67939593 | − | LTR16E2 |
| chr6 | 19021536-19022021 | − | LTR16E2 |
| chr6 | 19135247-19135444 | − | LTR16E2 |
| chr6 | 40070377-40070702 | + | LTR16E2 |
| chr6 | 40300304-40300799 | − | LTR16E2 |
| chr6 | 60594678-60594978 | − | LTR16E2 |
| chr6 | 6805623-6805991 | + | LTR16E2 |
| chr6 | 83837252-83837814 | − | LTR16E2 |
| chr6 | 89133415-89133558 | + | LTR16E2 |
| chr7 | 11739782-11740240 | − | LTR16E2 |
| chr7 | 155103317-155103722 | − | LTR16E2 |
| chr7 | 54971758-54972290 | + | LTR16E2 |
| chr7 | 79475937-79476354 | + | LTR16E2 |
| chr7 | 80270105-80270341 | − | LTR16E2 |
| chr8 | 112888397-112888944 | − | LTR16E2 |
| chr8 | 129415595-129415736 | − | LTR16E2 |
| chr8 | 137831154-137831283 | + | LTR16E2 |
| chr8 | 40453930-40454038 | − | LTR16E2 |
| chr8 | 68703397-68703889 | + | LTR16E2 |
| chr8 | 93475899-93476093 | + | LTR16E2 |
| chr8 | 94025417-94025908 | + | LTR16E2 |
| chr9 | 10681771-10682182 | − | LTR16E2 |
| chr9 | 14794135-14794403 | − | LTR16E2 |
| chr9 | 14794441-14794584 | − | LTR16E2 |
| chr9 | 32702167-32702707 | − | LTR16E2 |
| chr9 | 97790167-97790321 | − | LTR16E2 |
| chrX | 124945736-124946265 | − | LTR16E2 |
| chrX | 44101014-44101550 | + | LTR16E2 |
| chrX | 98492149-98492523 | + | LTR16E2 |

### Example 3: Effect of ASO-1 and ASO-3 on Cell Cycle States

Characterization of the cell cycle states of FB, MyoFB, and MyoFB-Scr, also revealed the differences between FB and MyoFB. Cell cycle states were characterized by analyzing the specific markers for cell cycle stages using the Seurat function for snRNA-seq analysis. As shown in FIG. 6H, cell cycle analysis showed that the majority of MyoFB and MyoFB-Scr were in G1, while FB were dividing more actively, suggesting that MyoFB were less proliferative than FB. Furthermore, transfection with scramble control did not change the cell cycle state of MyoFB.

To further characterize the difference between the MyoFB Scr cell population and MyoFB cell population treated with ASO-1 or ASO-3, cell cycle states of the different cell populations were analyzed. As shown in FIG. 6V, ASO-1 or ASO-3-transfected myofibroblasts were at G2M/S cell cycle states while myofibroblasts treated with ASO-scramble control were mostly in G1 state.

These results indicate that ASO-transfected myofibroblasts were not only different in their expression of fibrosis markers shown in Example 2, but they also shifted from G1 cell state to be mostly in G2M/S cell cycle states, compared to MyoFB Scr. Further, these results indicate that ASO-transfected myofibroblasts manifested a cell cycle state that was similar to healthy state observed in fibroblasts, and the ASO transfection transformed treated myofibroblasts to be more in the G2M/S cell cycle state than G1 (FIG. 6H and FIG. 6V).

### Example 4: ASO-1 and ASO-3 Treatment Induced Cell Reprogramming

As a cell goes through (partial) cellular reprogramming, the chromatin around which the DNA is wrapped presents a significantly more open state, which was evaluated by ATAC-seq discussed herein. The compartments into which the genome is organized are less defined, and insulation score goes up, which was evaluated by micro-C and CTCF discussed herein.

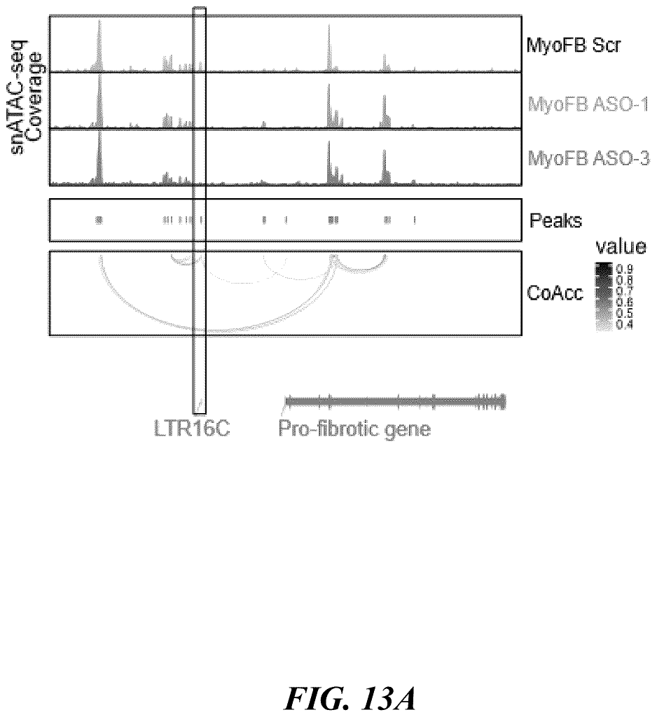

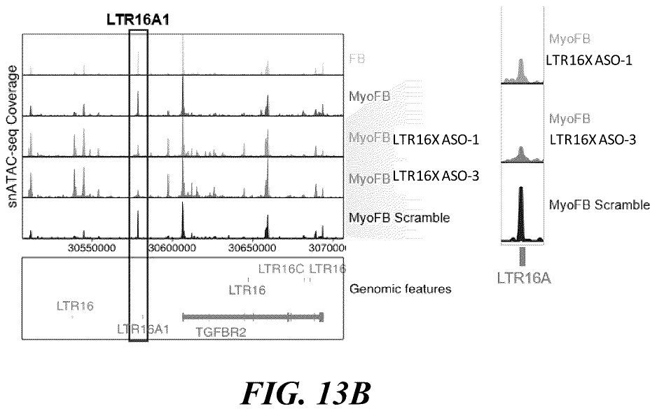

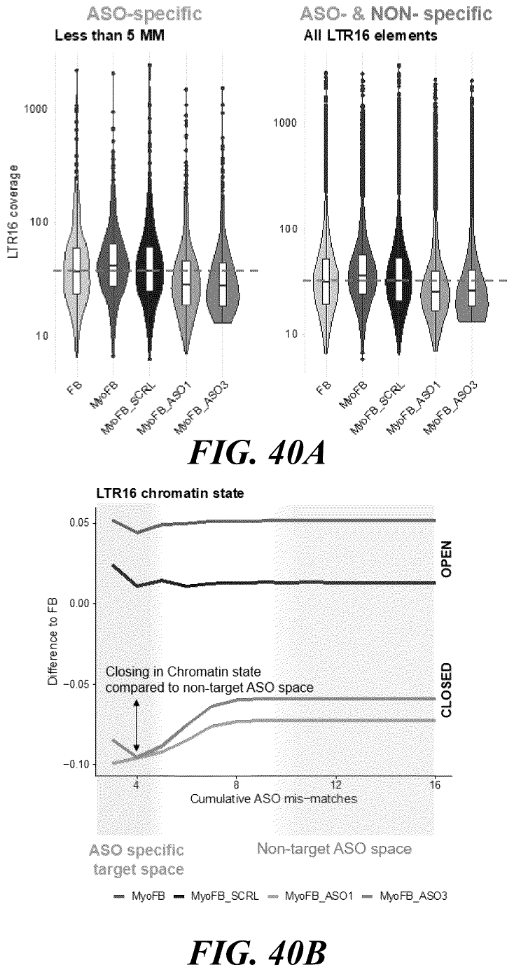

The downregulation of target transcripts encoded by LTR16X subfamilies shown in Example 2 may drive chromatin rearrangement. Thus, to further investigate this, single nuclear Assay for Transposase-Accessible Chromatin using sequencing (snATAC-seq) was performed to determine the effect of ASOs on chromatin accessibility. snATAC-seq was performed with myofibroblasts treated with Scramble (Scr), ASO-1, or ASO-3. To analyze snATAC-seq data, Raw FASTQ files were mapped to the human genome (GRCh38) using CellRanger-atac (v. 7.1.0). Subsequent data analysis was performed in R (v4.2.2) using ArchR (v1.0.1). Peak calling was performed using MACS2. Peak co-accessibility was performed in ArchR with a correlation threshold of 0.4. As shown in FIG. 40A, pseudobulk snATAC-seq revealed a decrease in coverage of transcripts encoded by LTR16 elements in both ASO specific target space (4 or less mismatches) and all LTR16 elements in myofibroblasts treated with ASO-1 or ASO-3 compared to fibroblast or myofibroblast with no treatment. As shown in FIG. 13A and FIG. 40B, snATAC-seq revealed that the chromatin on a transposable element locus (LTR16C) putatively targeted by the ASOs is closed when compared to myofibroblast treated with scramble (Scr, Scrl) control. The chromatin in the non-target space also remained open in myofibroblasts treated with ASOs compared to myofibroblast treated with scramble control (FIG. 40B), while the target space had a tendency to remain closed, which is different from the overall tendency of the chromatin to become opened genome-wide upon ASO treatment. Thus, the chromatin of an LTR16A1 locus, which is within the ASO-targeted loci, was also found to be closed in myofibroblasts treated with ASOs, compared to myofibroblasts treated with scramble control (FIG. 13B). The impact on chromatin accessibility and gene expression highlighted a possible cell state reprogramming.

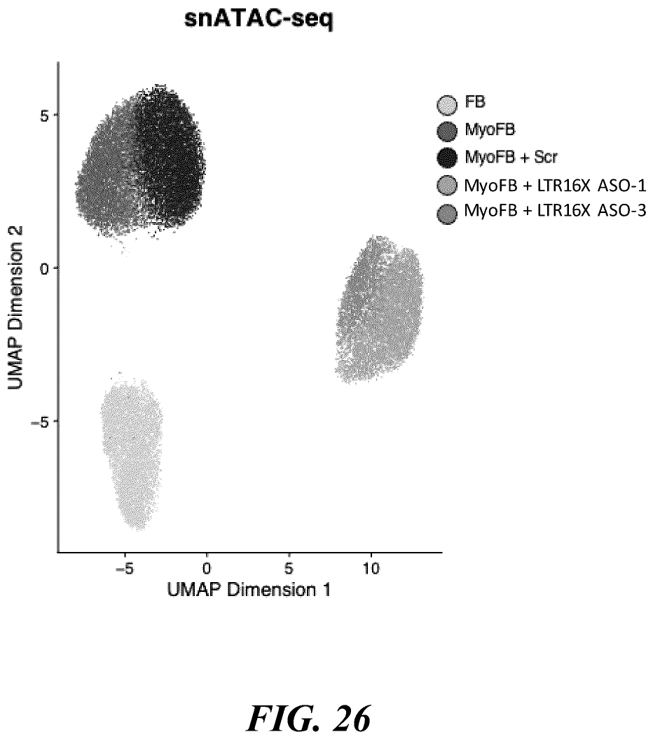

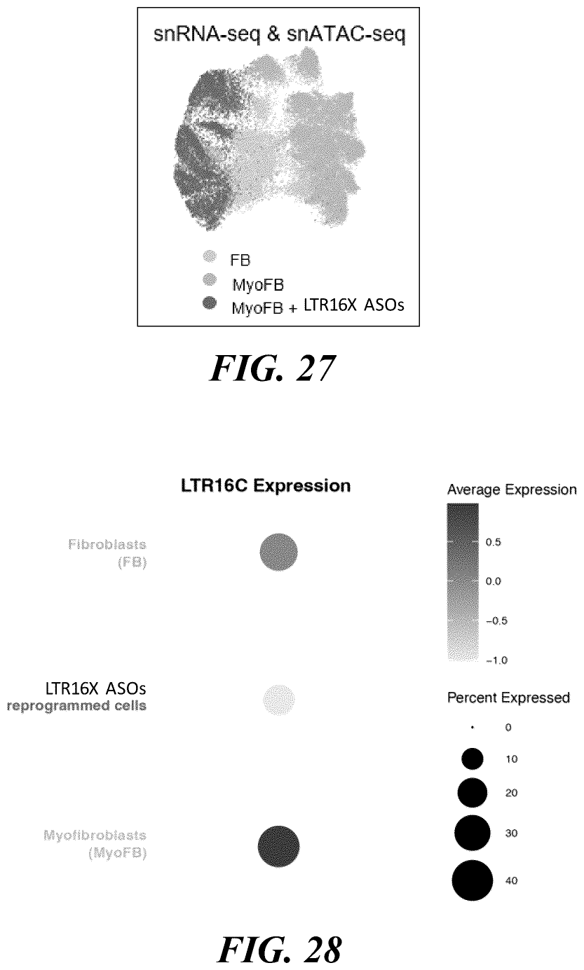

The snATAC-seq data were further analyzed to investigate possible epigenetic reprogramming upon ASO-1 or ASO-3 treatment. As shown in FIG. 26, snATAC-seq showed MyoFB+ASO-1 and MyoFB+ASO-3 clustered equally far away from fibroblast and myofibroblast, suggesting a different cell state (e.g., partially reprogrammed cells). Integration of snATAC-seq and snRNA-seq data also revealed distinct clusters representing fibroblasts, myofibroblasts, and ASO-transfected myofibroblasts (FIG. 27). ASO-treated myofibroblasts further exhibited reduced LTR16C expression compared to fibroblast and myofibroblast as shown in FIG. 28, suggesting an induction of a cell state that is different from fibroblast or myofibroblast upon ASO treatment.

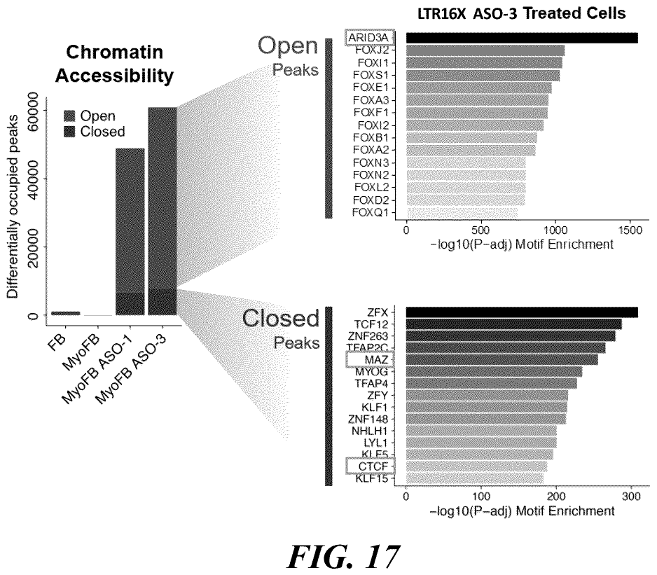

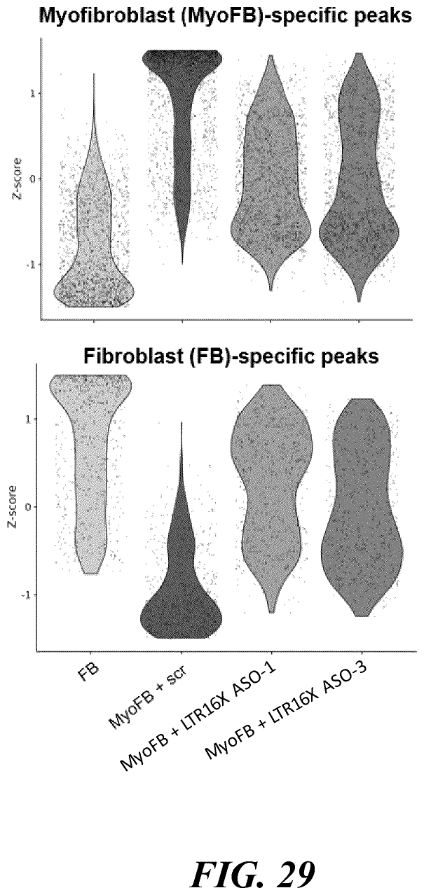

Analysis of myofibroblast and fibroblast specific peaks also showed that MyoFB+ASO-1 and MyoFB+ASO-3 led to closed myofibroblast-specific peaks and open fibroblast-specific peaks (FIG. 29), suggesting that MyoFB+ASO-1 and MyoFB+ASO-3 reprogrammed cells with a chromatin state that diverged from myofibroblasts. Additionally, the number of open ATAC-seq peaks substantially increased in MyoFB+ASO-1 and MyoFB+ASO-3 compared to myofibroblast or fibroblast (FIG. 17), suggesting a general opening of the chromatin. Motif enrichment for transcription factors relevant to cell state was found in the corresponding peaks (e.g., ARID3A, MAZ, CTCF).

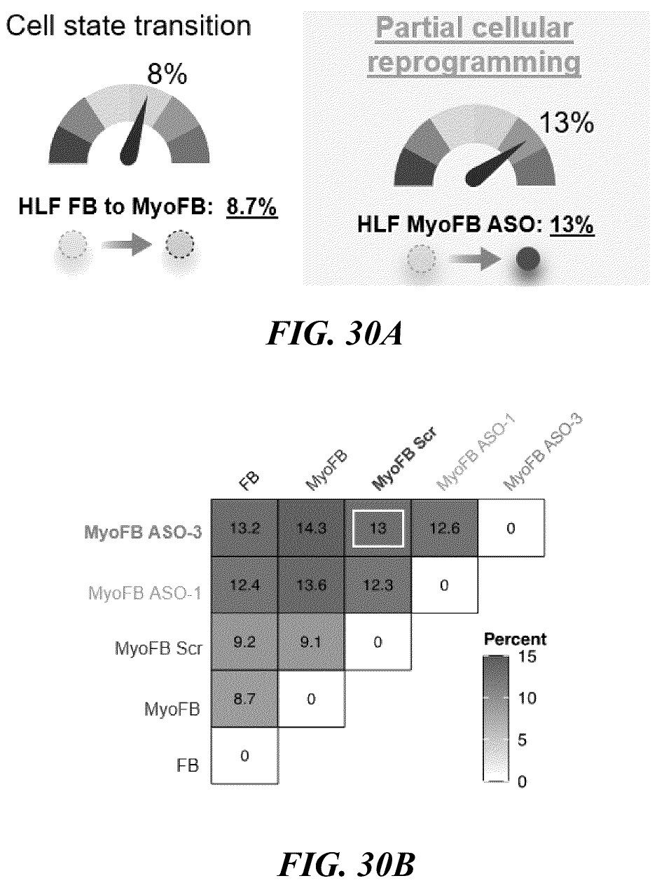

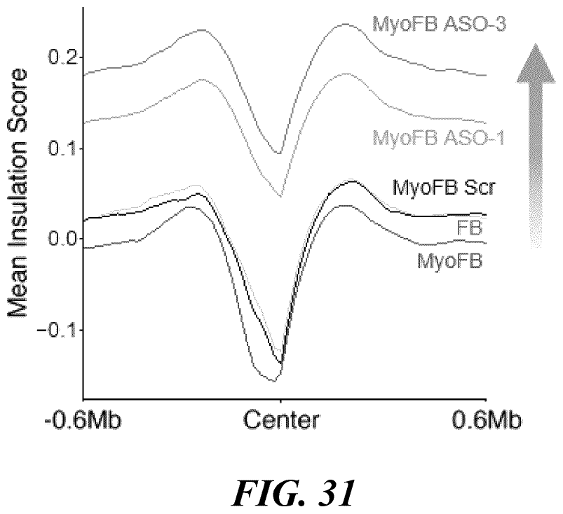

Next, micro-C was performed to resolve 3D genome interactions of ASO-treated myofibroblasts, and to further investigate whether there is cell reprogramming upon ASO-1 or ASO-3 treatment. As shown in FIG. 30A and FIG. 30B, substantial 3D genome rearrangement was identified upon treatment with ASO-1 and ASO-3, suggesting partial cell reprogramming. The percentage were calculated by measuring how many 10 kb bins out of the total in the genome have transitioned from an “A” to a “B” compartment state or from a “B” to a “A” compartment state, wherein the compartment state represent chromatin cell state with “A” refers to more opened chromatin while “B” refers to more closed chromatin. Furthermore, as shown in FIG. 31, micro-C revealed loosening of chromatin structure with ASO-1 or ASO-3 treatment, as measured by increase in insulation score, suggesting increased chromatin plasticity upon ASO-1 or ASO-3 treatment.

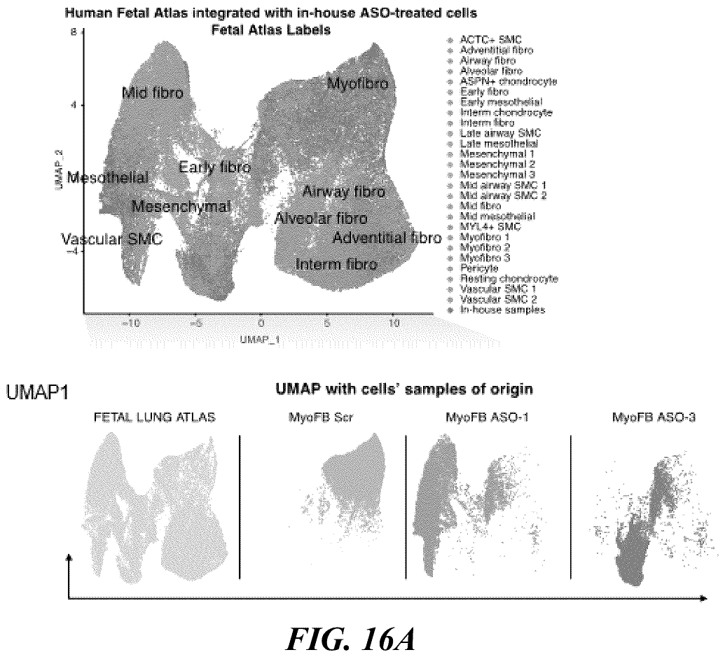

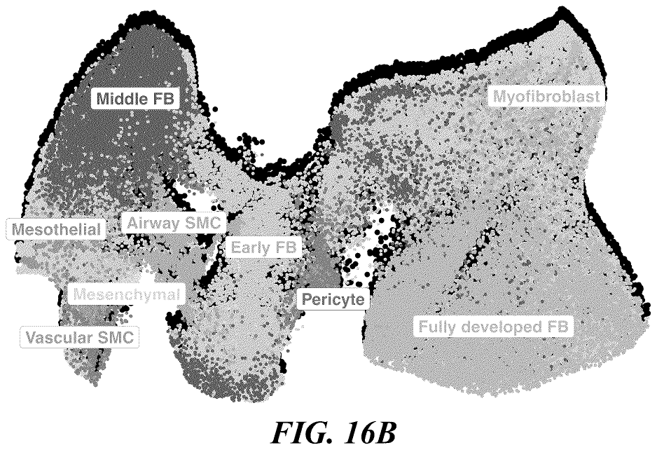

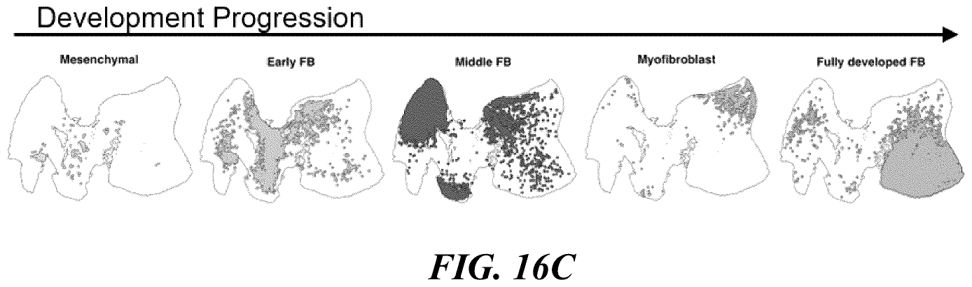

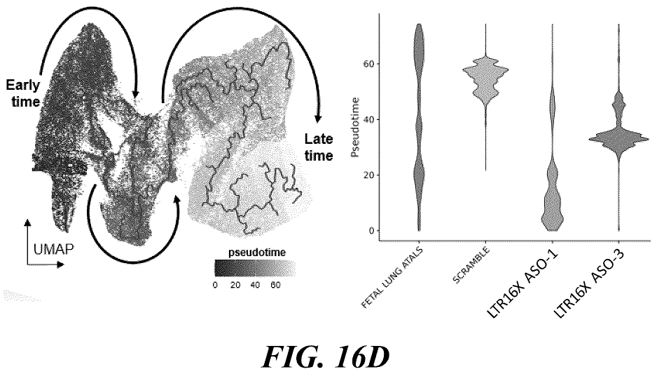

To assess the cell state and identity of the ASO-1 or ASO-3 reprogrammed cells, snRNA-seq analysis of primary HLF differentiated to MyoFB and treated with LTR16X ASOs was integrated with human lung fetal atlas (He et al., Cell 2022). For use of the human lung fetal atlas data, raw FASTQ files were downloaded from NCBI SRA repository and mapped to the same genomes and annotations used for the in-house data using CellRanger (v. 7.1.0). Subsequent data analysis was performed in a similar manner as in-house data. Integration with in-house snRNA-seq data was performed using LIGER (parameters used: k=20 and lambda=1). For HLF in-vitro snRNA-seq experiment, raw FASTQ files were mapped to the human genome (GRCh38) using CellRanger (v. 7.1.0) with annotation for Gencode genes (v43) and newly discovered lncRNAs. For the in-vivo mouse lung snRNA-seq experiment, raw FASTQ files were mapped to the mouse genome GRCm39 using celescope (v1.1.6) with annotation for ENSEMBL genes (v99). For both experiments, ambient contamination from residual cytoplasm was reduced using SoupX. Subsequent data analysis was performed in R (v. 4.2.2) using Seurat (v. 4.3.0). Raw UMI counts were normalized with a scale factor of 10,000 UMIs per cell and subsequently natural log transformed. The top 2000 most variable genes were then used to perform a PCA (with 50 principal components, PC). 30 PCs were used to identify neighboring cells and to perform UMAP. Plots were generated using ggplot2 and SCpubr libraries. As shown in FIGS. 16A-16C, when integrating in-house snRNA-seq on ASO-treated HLF with human fetal atlas samples, ASO-1 and ASO-3 treated myofibroblasts tended to cluster with early/mid fibroblasts. ASO-1 treated myofibroblasts clustered with mesothelial, mid and early fibroblast cells. ASO-3 treated myofibroblasts clustered with early fibroblast and mesenchymal cells. Unsupervised pseudotime analysis also showed that ASO-treated myofibroblasts were reprogrammed toward an early stage of lung development, as shown in FIG. 16D. These data collectively showed that LTR16C-ASO treated cells may be reprogrammed into early fibroblast precursor cells.

Since partial cellular reprogramming may reduce aging rates and increase lifespan, the transcriptome of ASO-1 or ASO-3 treated myofibroblasts was further investigated for any changes in genes associated with aging. snRNA-seq data from human lung fibroblasts (HLF) treated with TGFβ plus serum starvation were transfected with ASO-scramble control (MyoFB+Scr), ASO-1 (MyoFB+ASO-1) or ASO-3 (MyoFB+ASO-3).

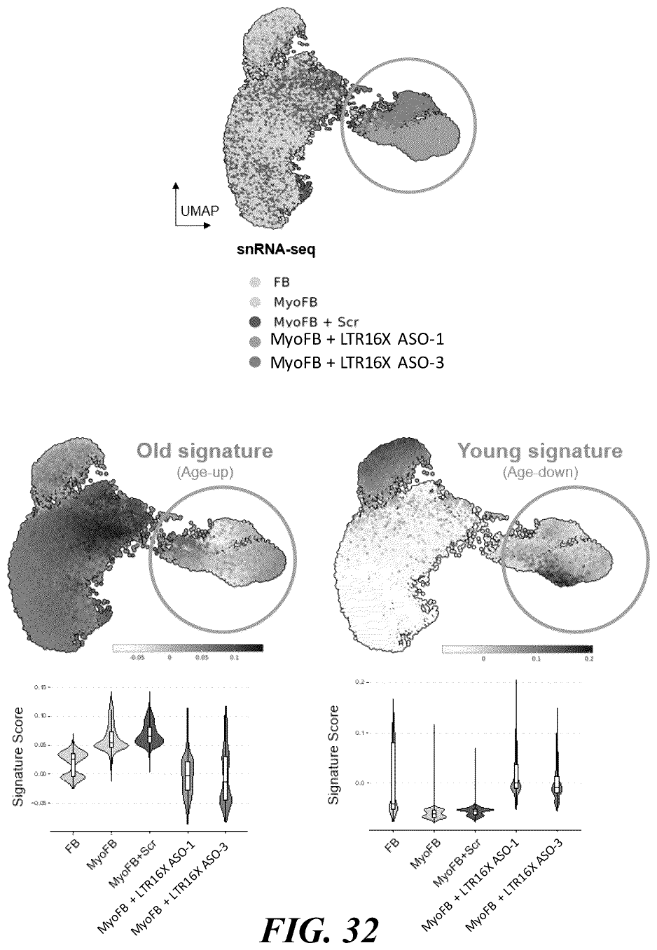

Old and young signature were evaluated. Genes from young and old signature are provided by Chow et al. 2021, Nature Comms. The signatures from the publication (AgeUp and AgeDown respectively) can be found in the Supplementary table 7 of Chow et al. As shown in FIG. 32, MyoFB+ASO-1 and MyoFB+ASO-3 had decreased old signature compared to MyoFB+Scr. Conversely, MyoFB+ASO-1 and MyoFB+ASO-3 had increased young signature compared to MyoFB+Scr, revealing possible transcriptomic rejuvenation.

Thus, collectively, the epigenomic and transcriptomic results suggest that upon treatment of ASO-1 or ASO-3, myofibroblasts may be reprogramed into mesenchymal progenitors, or early/mid fibroblasts to rejuvenate the cell to a younger, healthier cell state compared to a disease-related cell state.

Further Cut&Run/ChIP data are generated on fibroblasts and myofibroblasts untreated or treated with ASO-1 or ASO-3 to further assess chromatin status of the LTR16C locus. Analysis with PRO-seq, bulk RNA-seq, IC50 is performed to provide additional insight into pathways and genes associated with LTR16C locus.

### Example 5: ASO-1 and ASO-3 Treatment in Mice

Due to the high conservation of LTR16 targets across species, the human candidate ASOs are expected to be pharmacologically active in both humans and mice. Therefore, human candidate ASOs (ASO-1 and ASO-3) were used in mice.

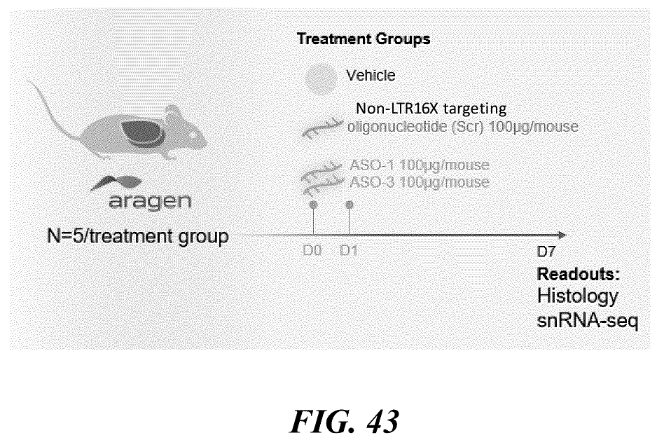

Wild type C57BL/6J mice (e.g., N=5/treatment group) were administered via aerosol or microsprayers with either vehicle, ASO-scramble control (100 μg/mouse), ASOs (ASO-1 or ASO-3 at 100 μg/mouse), mapped to chr3: 45,818,736-45,818,762, at day 0 and day 1, and samples were taken at day 7 for analysis, as illustrated in the experimental schema in FIG. 8A and FIG. 43. After the two-dose treatment at days 0 and 1, body weights of the treated mice were measured every day. As shown in FIG. 8B and FIG. 8C, mice treated with ASO-1 or ASO-3 showed no statistically significant weight changes during multiple days after treatment (e.g., 7-8 days), suggesting that no substantial toxicity of ASO-1, or ASO-3 could be detected in the systemic level in vivo.

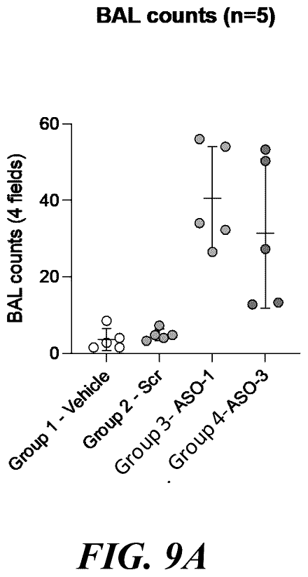

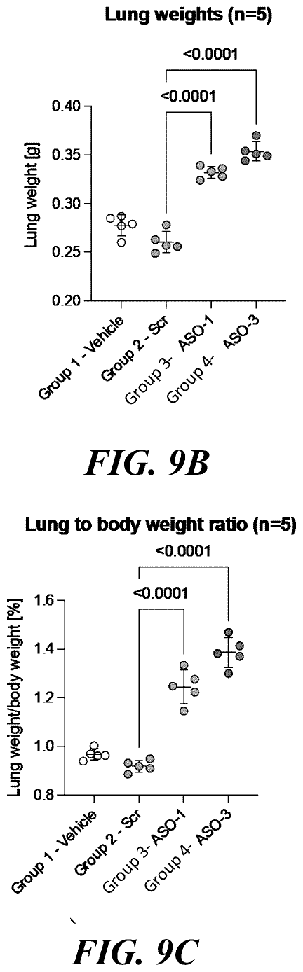

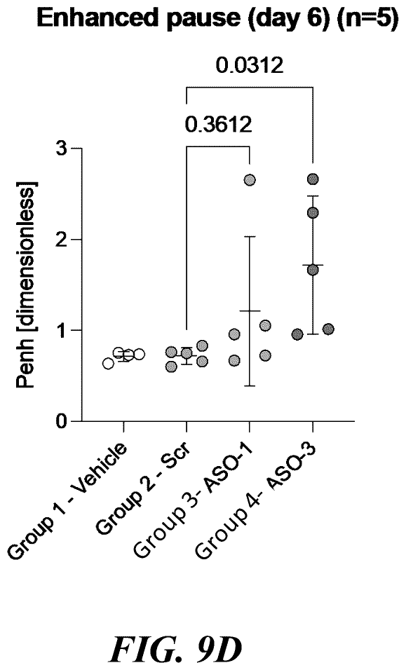

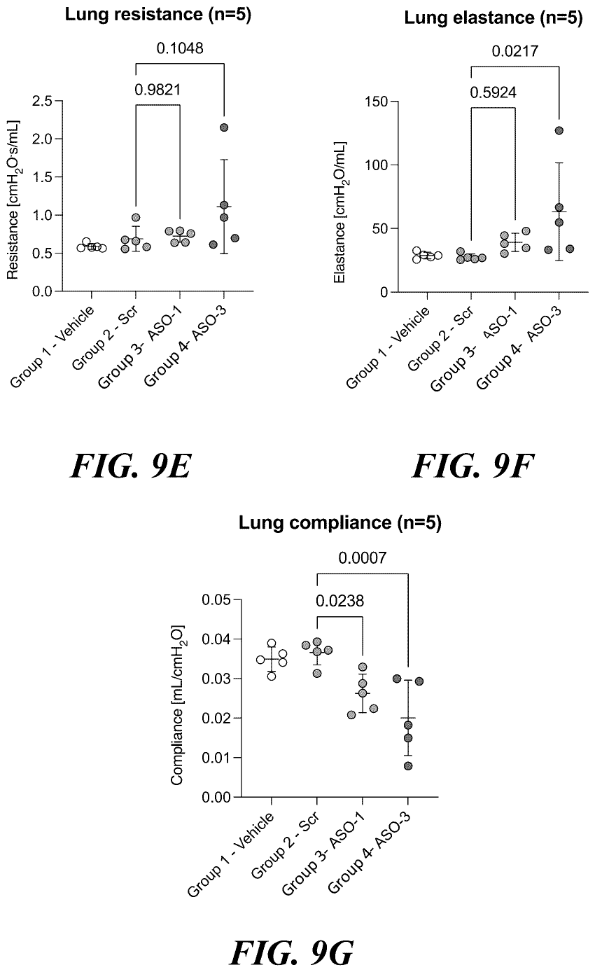

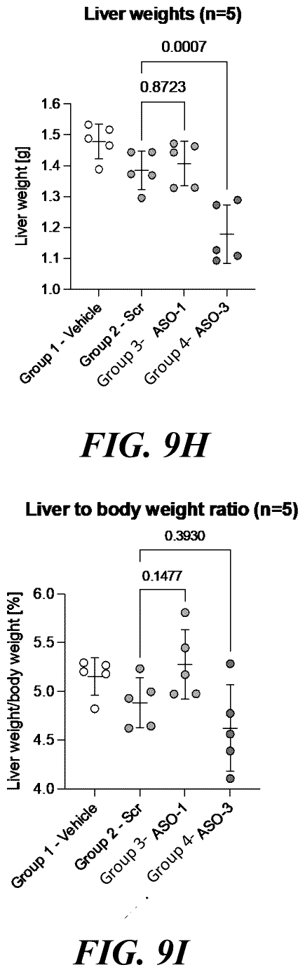

Various assays to determine the effect of ASOs (ASO-1 or ASO-3) on in vivo lung tissue or cells were also conducted in mice treated with ASO-1 or ASO-3, or ASO-scramble control (Scr). In bronchoalveolar lavage (BAL) cell analysis, bronchoalveolar lavage (BAL) fluid was collected from isolated lungs obtained from the mice treated with ASO-1 or ASO-3, or ASO-Scr or vehicle as a control. BAL cell counts (total leukocyte and differential counts) were counted manually under a microscope, and BAL from lung tissues obtained from the mice treated with ASO-1 or ASO-3 showed increased BAL counts compared to vehicle treated or ASO-Scr treated lung tissues (FIG. 9A). Mice treated with ASO-1 or ASO-3 also had higher lung weights and higher lung to body weight ratio than mice treated with ASO-scramble control or vehicle (FIG. 9B and FIG. 9C, respectively). These results indicate potential inflammation in the lung (e.g., increased inflammatory cell numbers) treated with ASO-1 and ASO-3.

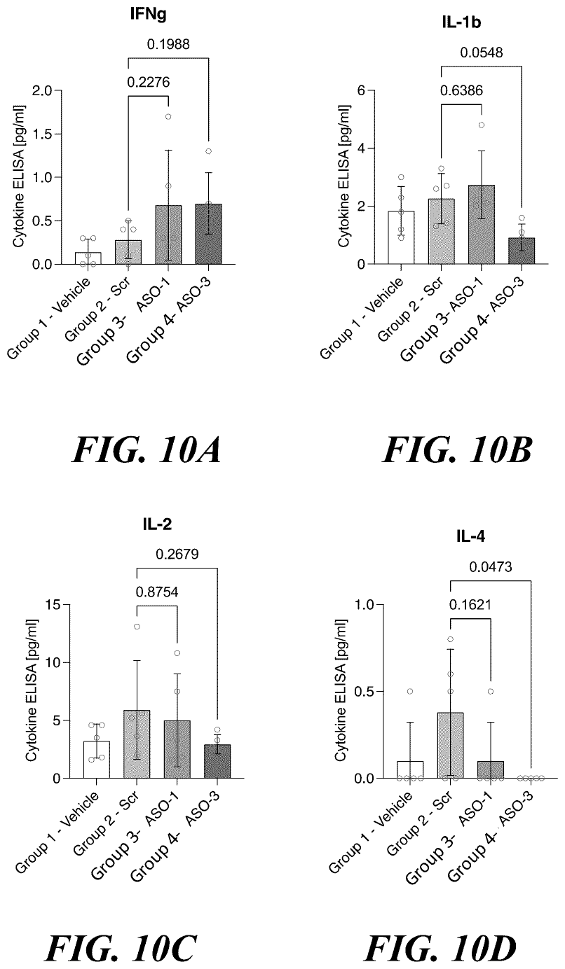

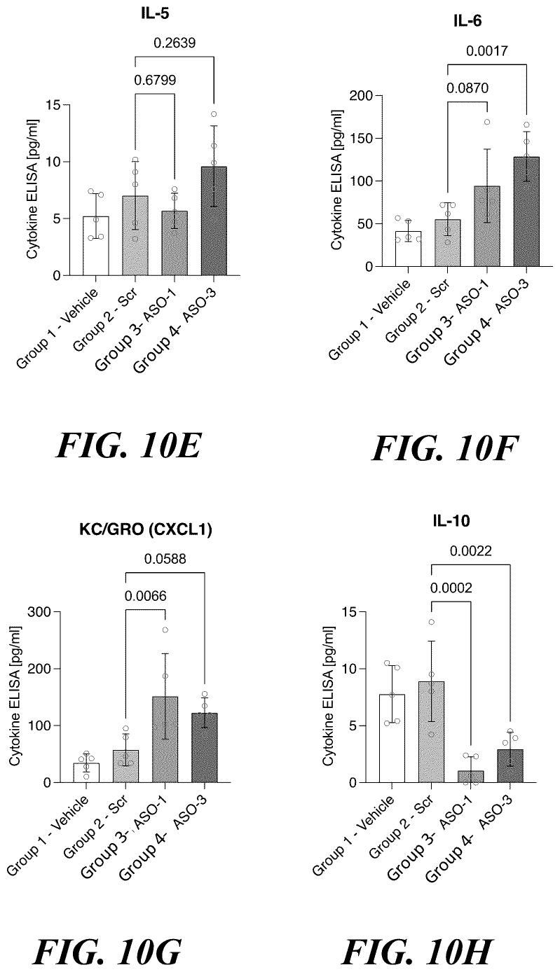

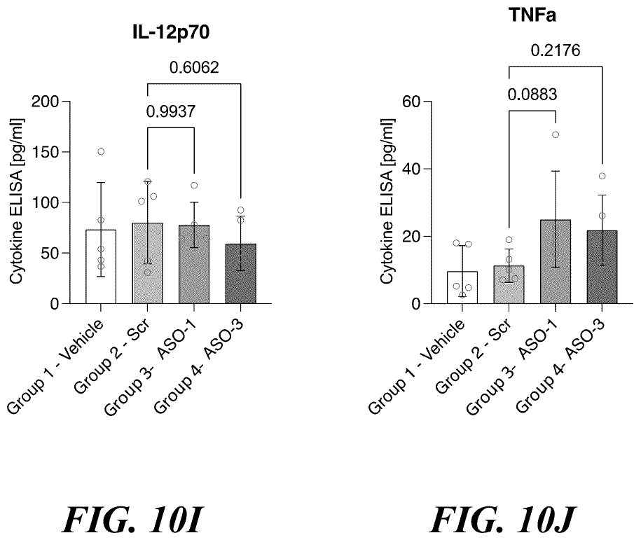

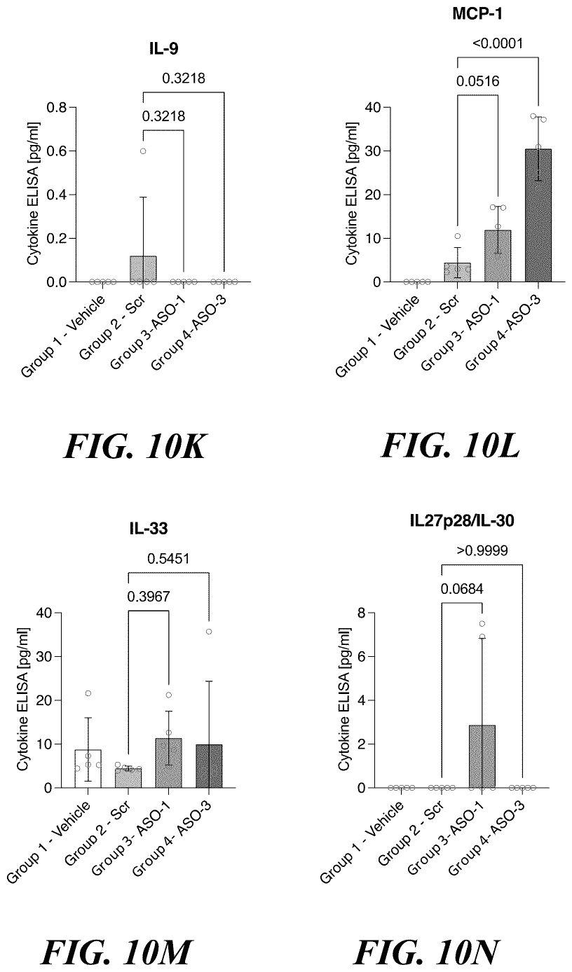

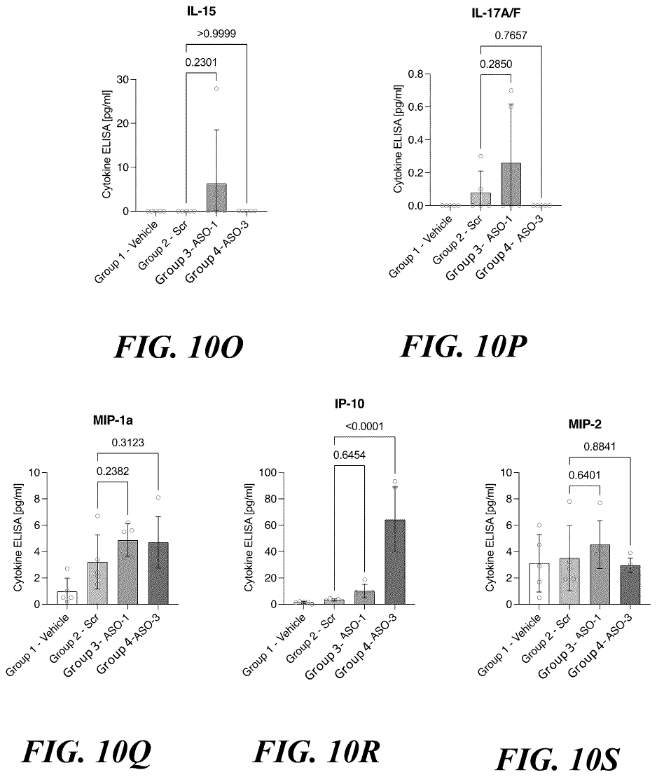

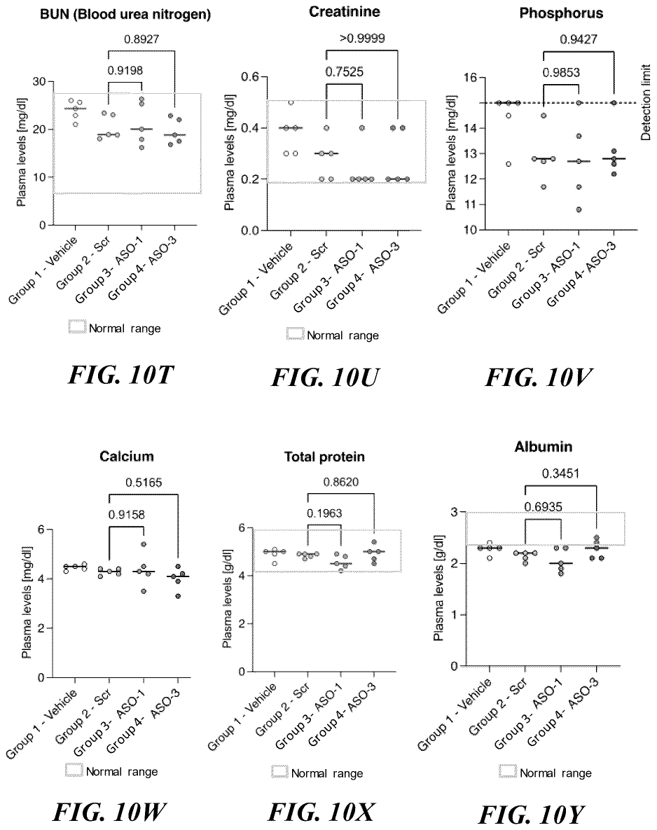

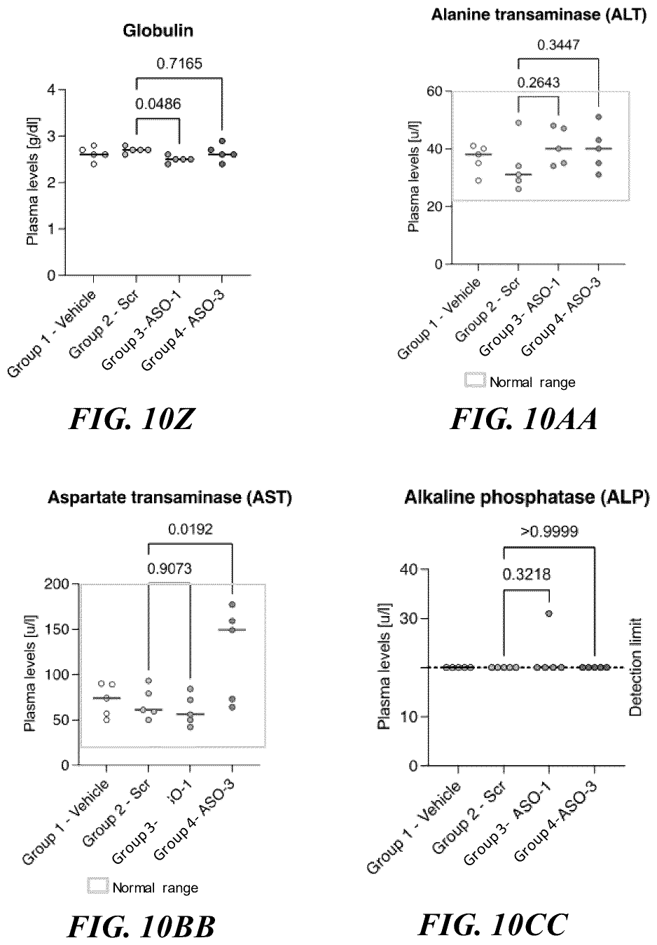

Functional lung capacity (enhanced pause (Penh)) of the mice treated with ASO-3 was measured by performing whole body plethysmography (WBP) to evaluate the changes in the shape of the airflow pattern entering and leaving, thereby measuring the airway reactivity. WBP was performed on mice with the Buxco system. In this experiment, mice treated with ASO-3 showed statistically significantly higher Penh compared to mice treated with ASO-Scr, suggesting airway hyper-responsiveness (FIG. 9D). In addition, lung resistance, lung elastance, and lung compliance were measured in the mouse treated with ASO-3 with a FlexiVent machine. As shown in FIG. 9E, there was no change in lung resistance among mice treated with vehicle, mice treated with ASO-Scr, mice treated with ASO-1 and mice treated with ASO-3. On the other hand, there was statistically significant increase in lung elastance in mice treated with ASO-3 (FIG. 9F), while there was statistically significant decrease in lung compliance in mice treated with either ASO-1 or ASO-3 group compared to ASO-scramble control group (FIG. 9G). Collectively, the various lung-related assays showed that treatment of ASOs induced changes in the lungs, such as its inflammatory cell number counts, size, and performance in naive mice. Additionally, the liver weights were also measured and normalized by body weight to test whether treatment of ASOs affect other organs function, such as liver. (FIG. 9H and FIG. 9I) It was found that mice treated with ASO-3 had statistically significant decreased in liver weight compared to mice treated with ASO-Scr. However, the liver to body weight ratio did not exhibit statistically significant change compared to mice treated with ASO-Scr. Furthermore, plasma biochemistry associated with liver function (e.g., Blood urea nitrogen, creatinine, phosphorus, calcium, total protein, albumin, globulin, alanine transaminase, and aspartate transaminase, and alkaline phosphatase) was measured with ELISA to see whether treatment of ASOs affect liver function. As shown in FIGS. 10T-10Z, 10AA-10CC, there was no statistically significant change in plasma biochemistry in mice treated with ASOs compared to mice treated with ASO-Scr, with the plasma biochemistry in mice falling within normal ranges. Normal values were obtained from reference data provided by Charles River Laboratories. Only aspartate transaminase level had statistically significantly increase in mice treated with ASO-3 compared to mice treated with ASO-Scr. Collectively, the data suggests that inhibition of LTR16C does not substantively affect liver size and function.

As treatment with ASO-1 and ASO-3 led to phenotypic changes of the lungs (e.g., increasing trend in inflammatory cell number counts, changes in lung size and performance), cytokine ELISA analysis on various hallmarks of inflammation (e.g., IFNg, IL-1b, IL-2, IL-4, IL-5, IL-6, CXCL1, IL-10, IL-12p70, TNFα, IL-9, MCP-1, IL-33, IL27p28/IL-30, IL-15, IL-17A/F, MIP-1a, IP-10, MIP-2) were performed (FIGS. 10A-10S) to test whether expression of specific inflammatory markers is also affected by ASO treatment. Treatment with ASO-3 markedly increased expression of some markers of inflammation and immune cell infiltration (e.g., MCP-1, IL-6, and IL-10) and decreased expression of cytokines associated with anti-inflammatory function (e.g., IL-4, IL-10). Treatment with ASO-1 also significantly increased expression of some inflammatory markers (e.g., IL-6, CXCL1), while decreasing expression of anti-inflammatory markers (e.g., IL-10).

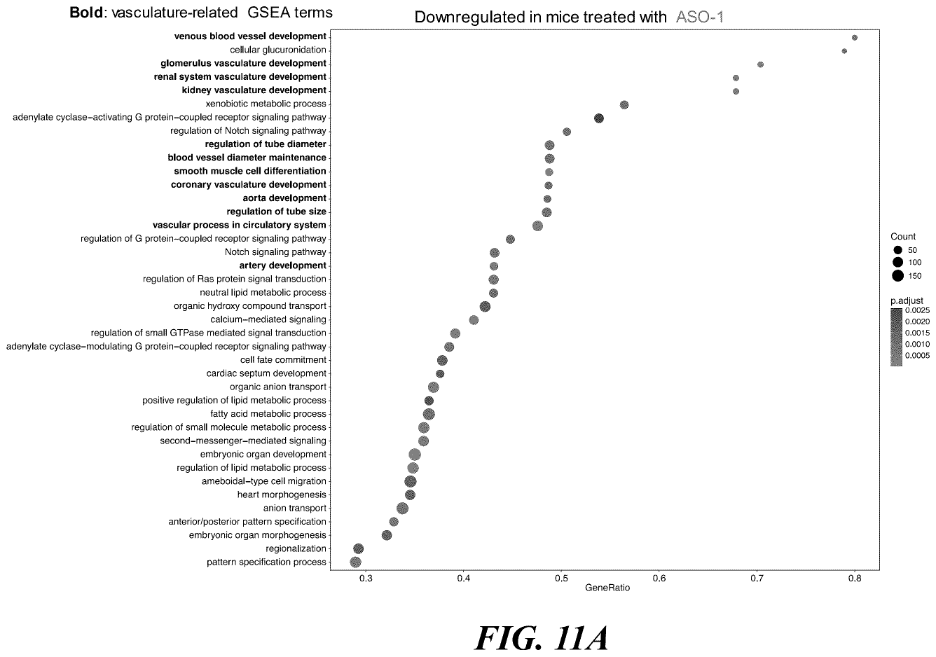

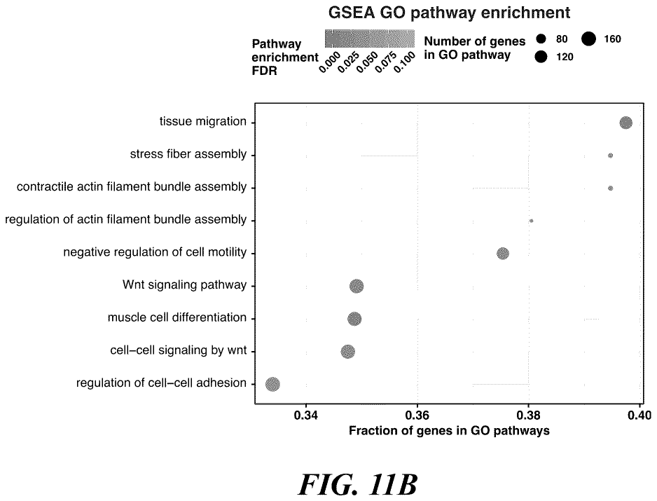

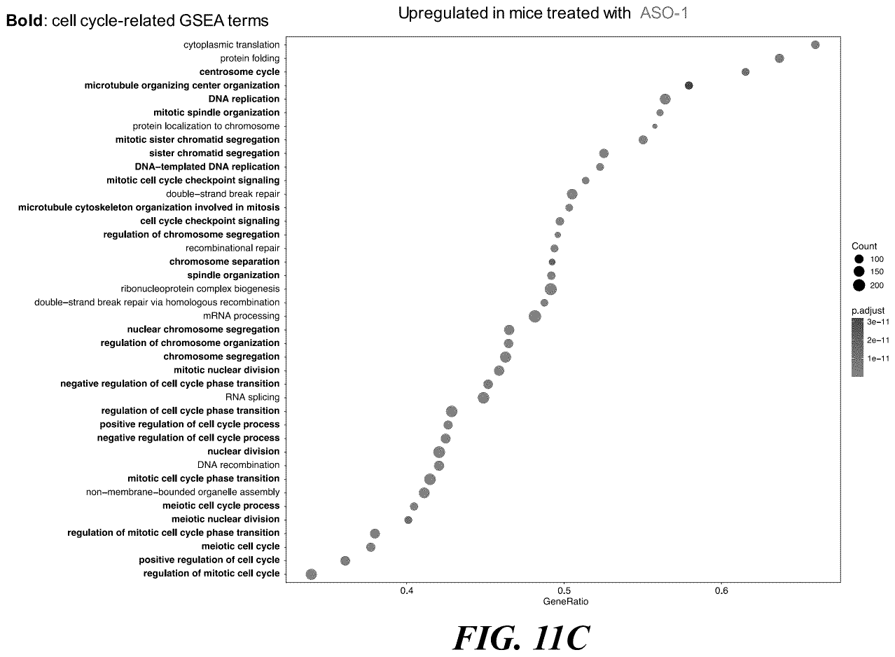

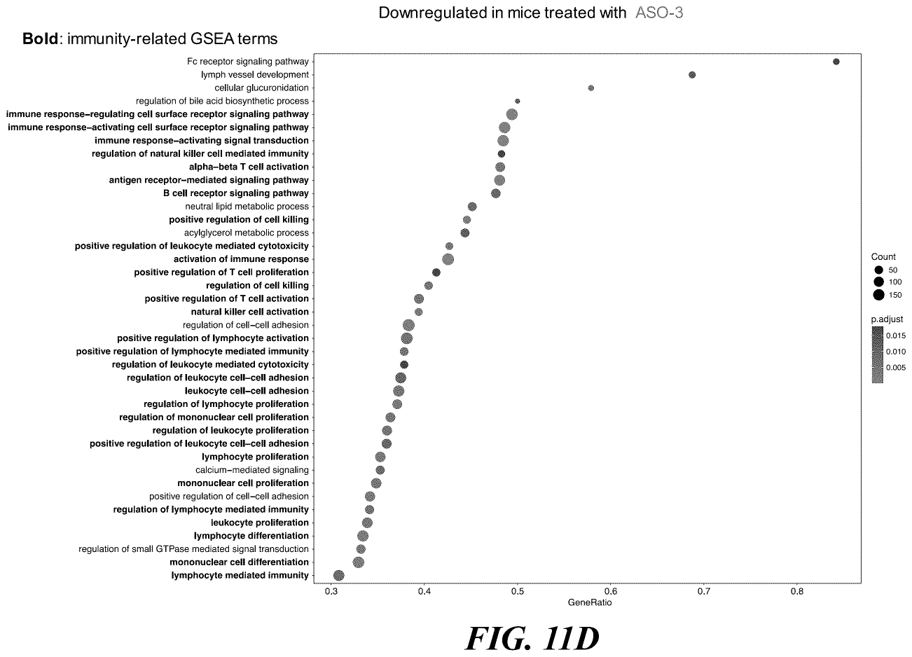

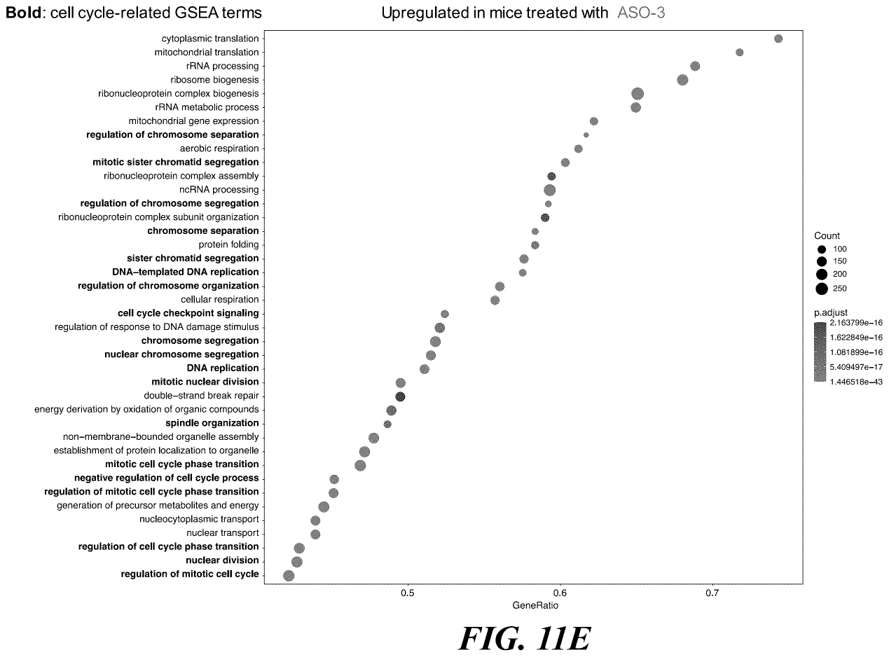

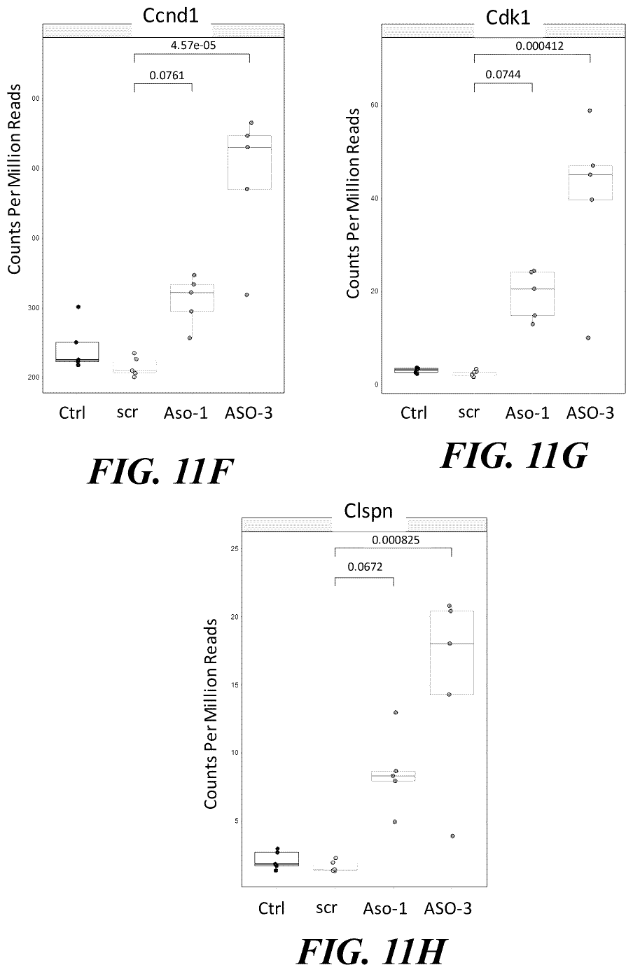

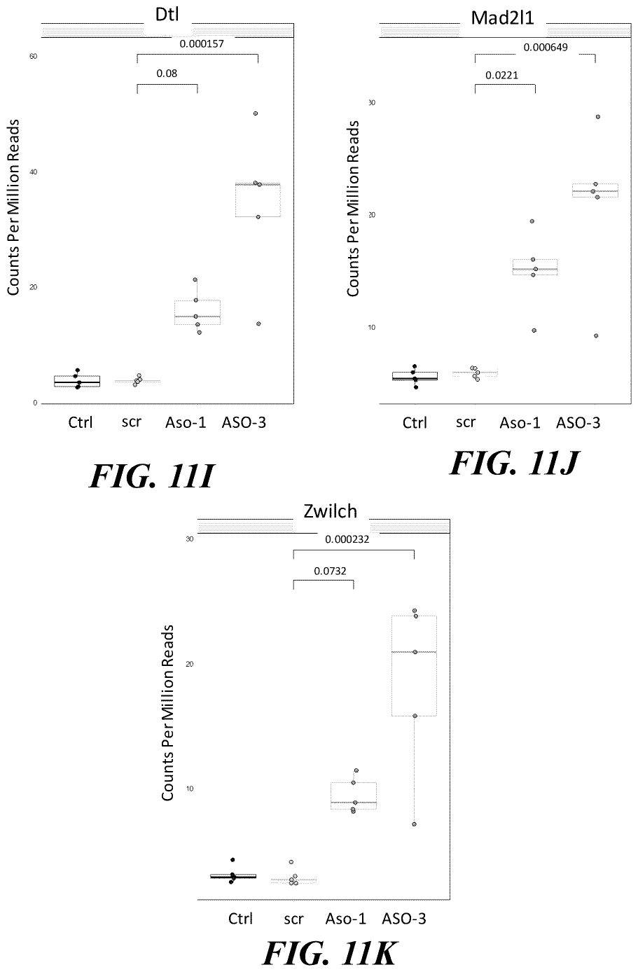

Information on the genes that were upregulated and downregulated in mice treated with ASOs compared to mice treated with ASO-Scr were obtained using RNA-seq data from lung tissues, and were further analyzed with Gene Set Enrichment Analysis (GSEA). GSEA was conducted to associated functions to the group of upregulated and downregulated genes. GSEA with genes downregulated in mice treated with ASO-1 compared to ASO-Scr showed that inhibition of LTR16C by ASO-1 may affect vasculature development, such as venous blood vessel development, glomerulus vasculature development, renal system vasculature development, and kidney vasculature development (FIG. 11A). GSEA gene ontology pathway enrichment analysis (FIG. 11B) showed that genes downregulated in mice may be associated to cell adhesion related to myofibroblasts. GSEA with genes downregulated in mice treated with ASO-3 compared to ASO-Scr also showed that inhibition of LTR16C by ASO-3 may affect immune response activation and leukocyte functions (FIG. 11D). GSEA with genes upregulated in mice treated with ASO-1 and ASO-3 showed other functional changes related to cell division and cell cycle transition (FIG. 11C and FIG. 11E). Markers of cell cycle was analyzed with the RNA-seq data and showed that upon treatment with ASO-1 or ASO-3 there was statistically significant upregulation of cell cycle genes, such as Cend1 (FIG. 11F), Cdk1 (FIG. 11G), Clapn (FIG. 11H), and Dt1 (FIG. 11I), Mad211 (FIG. 11J), and Zwilch (FIG. 11K) compared to ASO-Scr (scr). These data are consistent with the cell state analysis of MyoFB Scr cell population and MyoFB cell population treated with ASO-1 or ASO-3 described in Example 3.

These in vivo studies revealed that the ASOs were well tolerated by mice, and that the ASOs originally designed for a different species (e.g., humans) can lead to changes in lung phenotype, and expression of inflammatory and fibrosis markers in mice. In vivo studies with mouse disease model (e.g., pulmonary fibrosis mouse models) were further utilized to investigate the therapeutic effect of LTR16C-ASOs, as discussed in Example 7.

### Example 6: Effects of ASO-1 and ASO-3 in Naive Mice

Single-nucleus RNA-seq (snRNA-seq) was performed on snap-frozen biopsies collected from the lungs of the mice. Tissue dissociation was performed and snRNA-seq libraries were prepared at Singleron Biotechnologies (Köln, Germany) as described (https://www.ncbi.nlm.nih.gov/pmc/articles/PMC8100173/). The libraries were sequenced PE150 on a lane of a NovaSeq 6000.

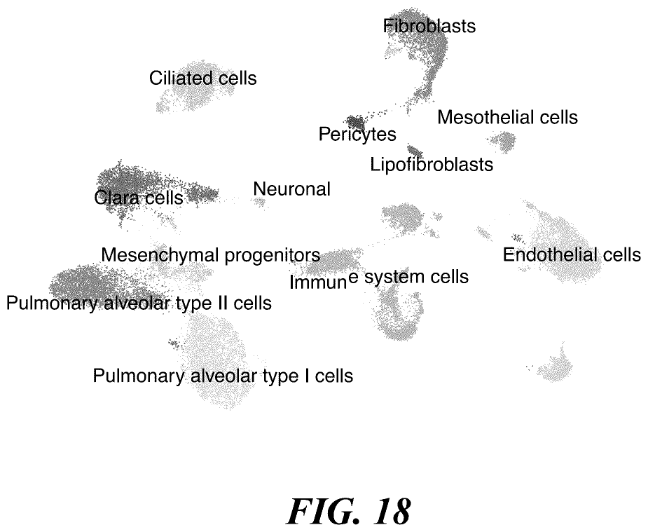

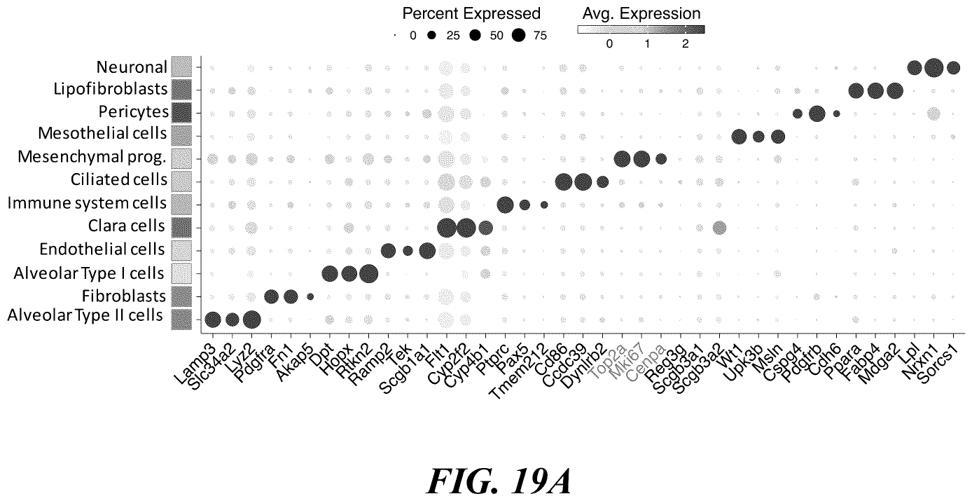

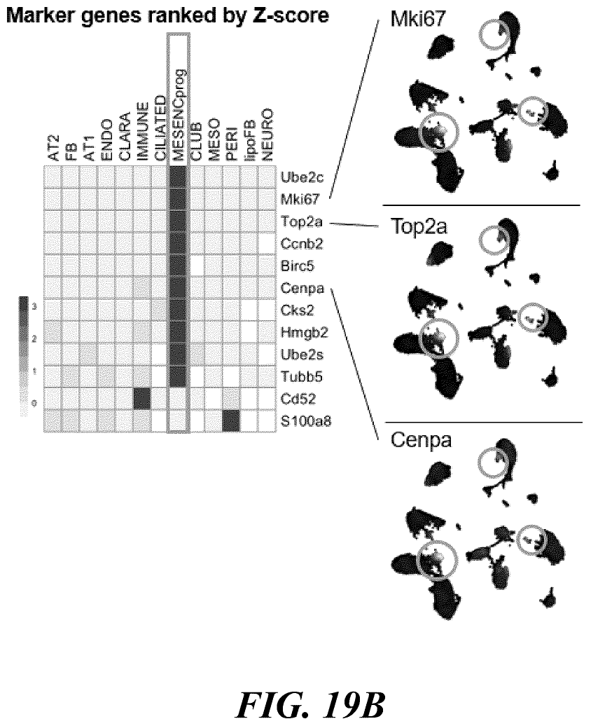

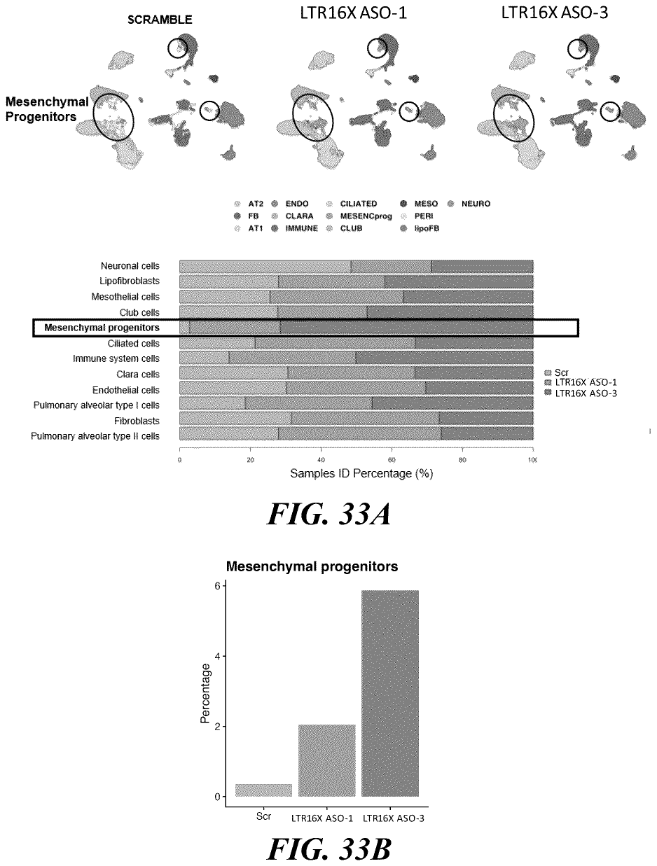

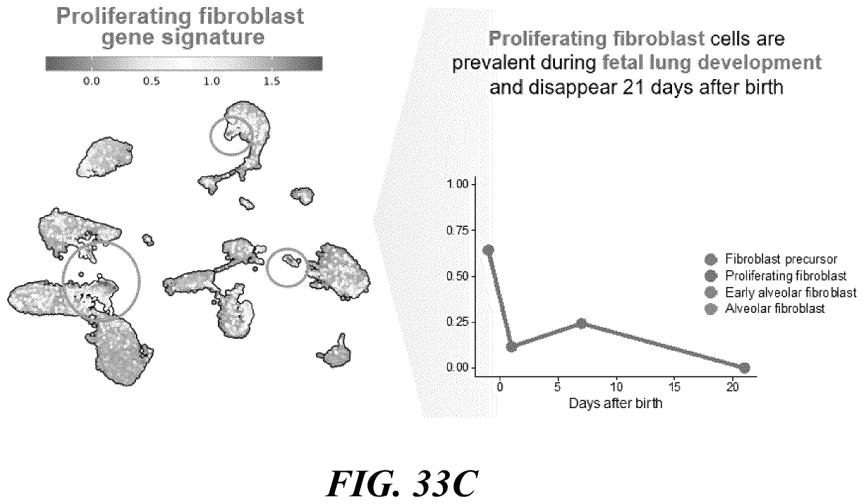

Cell type annotation (scType) on snRNA-seq was performed on lung biopsies from Scramble- or ASO-1 or ASO-3-treated naive mice and showed a population of mesenchymal progenitors (FIG. 18). Expression heatmap of top 3 marker genes characteristic for each identified population indicated that mesenchymal progenitors express Mki67, Top2a, and Cenpa (FIG. 19A and FIG. 19B). Further analysis of snRNA-seq data obtained from lung biopsies of scramble control or ASO-1 or ASO-3 treated mice revealed that ASO-treated cells were mesenchymal progenitors, as shown in FIG. 33A. Quantification of the number of mesenchymal progenitors showed that mesenchymal progenitors represent 2-6% of the total cells in lung biopsies of ASO-treated mice (FIG. 33B). These mesenchymal progenitors were also found to have a transcriptional profile with increased expression of a proliferating fibroblast gene signature compared to other cell types, as shown in FIG. 33C. The proliferating fibroblast gene signature was obtained from a public dataset (Zanini et al, Scientific Reports, 2024), and included 6646 upregulated genes in proliferating fibroblasts. Since the proliferating fibroblast cells are prevalent during fetal lung development and disappear 21 days after birth (FIG. 33C), the increased expression of a proliferating fibroblast gene signature in mesenchymal progenitor cells suggested that ASO treatment-specific mesenchymal progenitor cells may have a transcriptional profile equivalent to mouse fetal lung fibroblasts.

To further validate that in vivo ASO-1 or ASO-3 treatment may lead to reprogramming of cells, snRNA-seq performed on lung biopsies (n=2 animals/group) were integrated with a mouse lung reference public dataset (Xie et al., Cell Reports 2018). Raw FASTQ files were downloaded from NCBI SRA repository and mapped to the same genomes and annotations used for the in-house data using CellRanger (v. 7.1.0). Subsequent data analysis was performed in a similar manner as in-house data. Integration with in-house snRNA-seq data was performed using LIGER (parameters used: k=20 and lambda=1).

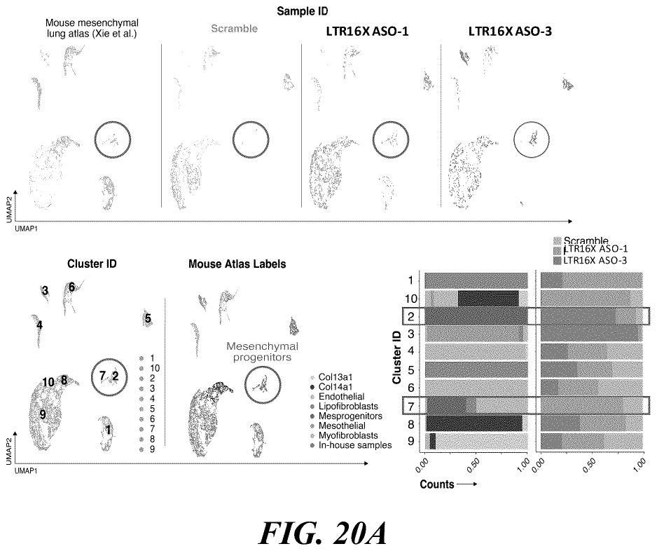

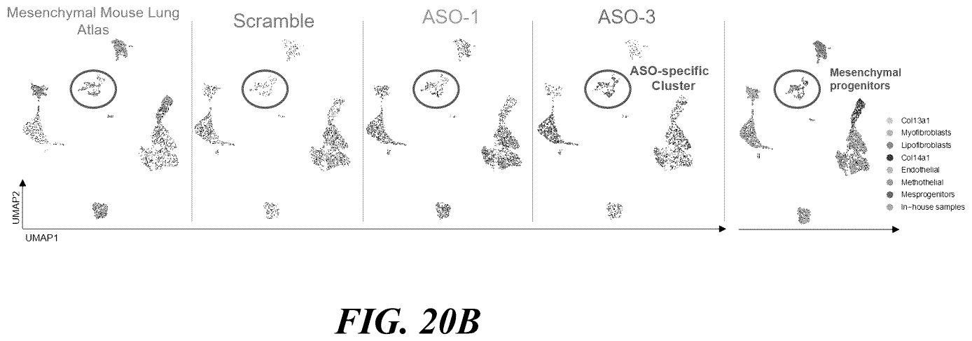

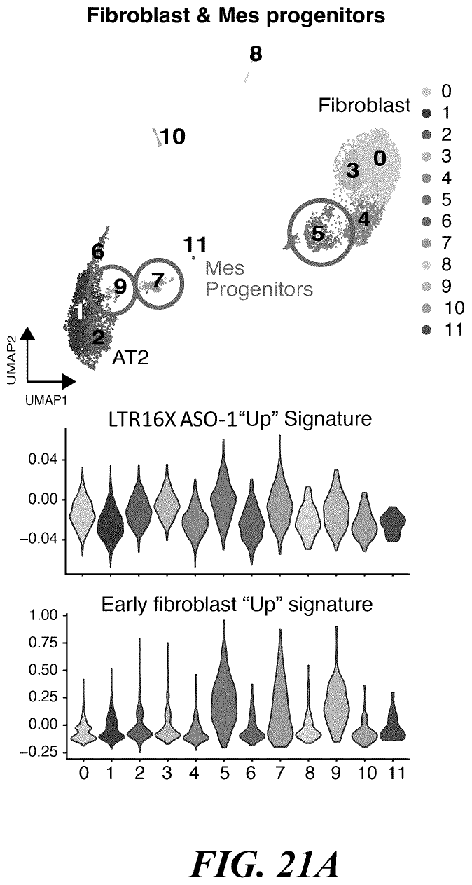

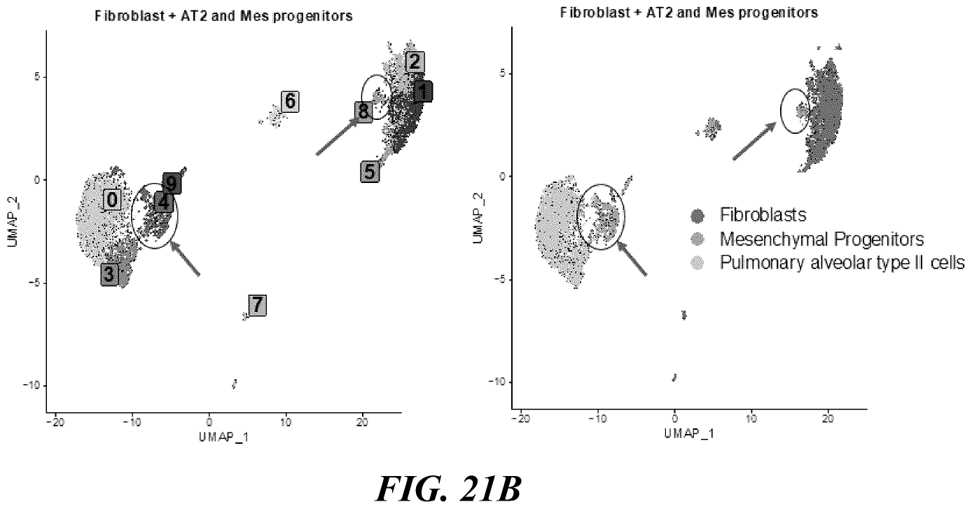

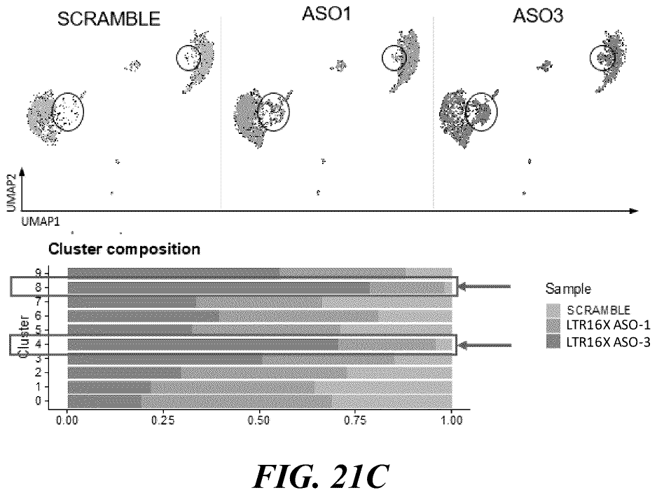

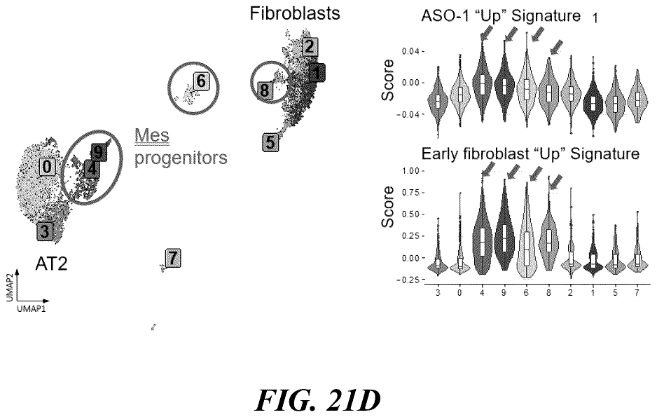

As shown in FIG. 20A and FIG. 20B, when integrating in-house in vivo snRNA-seq with the published mouse mesenchymal cell type dataset, a population specific to the ASO-treated tissues appeared. It colocalized with mesenchymal progenitors from the published data. When subselecting mesenchymal cell types from the in-house in vivo data and performing a UMAP analysis (FIG. 21A), the putative mesenchymal progenitors population (clusters 5, 7, 9) appeared to be located between fibroblasts (clusters 0, 3, 4) and AT2 (alveolar type II) (clusters 1, 2, 6) cells. This result remained consistent in another in vivo snRNA-seq analysis, as shown in FIG. 21B, wherein the analysis of mesenchymal-derived cells (e.g., mesenchymal progenitor cells, fibroblast, and AT2) showed mesenchymal progenitors to be clustered between fibroblast and AT2. The cluster composition showed that the circled transitional clusters (cluster 8, 4), representing mesenchymal progenitors, were primarily enriched in ASO-treated mice (FIG. 21C), suggesting partial reprogramming of myofibroblasts with the rise of this intermediate cell state upon treatment of ASO.

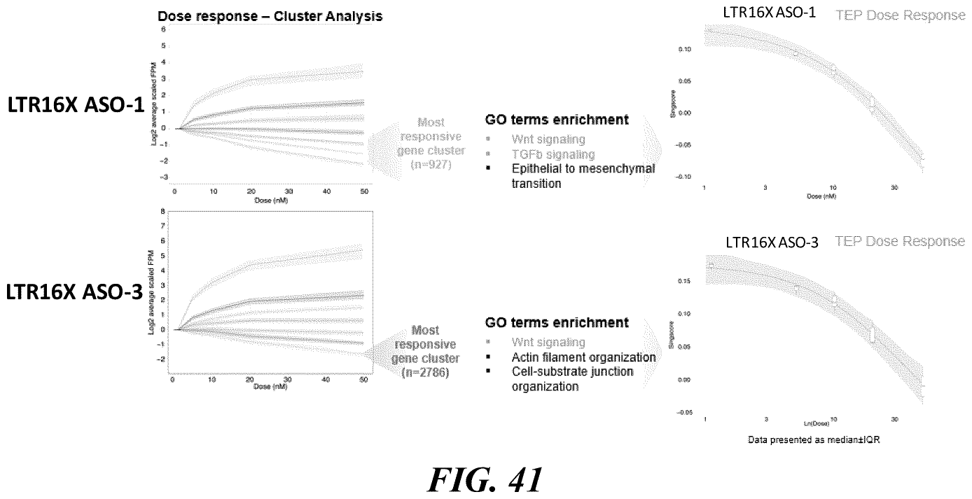

Furthermore, gene sets of interest (mouse orthologs of the ASO-1 engaged genes and the genes specific to early fibroblasts) were profiled for each cluster. The ASO engaged genes were derived from a set of genes that showed to be most responsive in myofibroblast treated with ASO-1 or ASO-3, and correlated with dose increases, as shown in FIG. 41. The mouse orthologs of ASO-1 engaged genes and genes specific to early fibroblasts were both upregulated in the mesenchymal progenitor cluster (FIG. 21A and FIG. 21D).

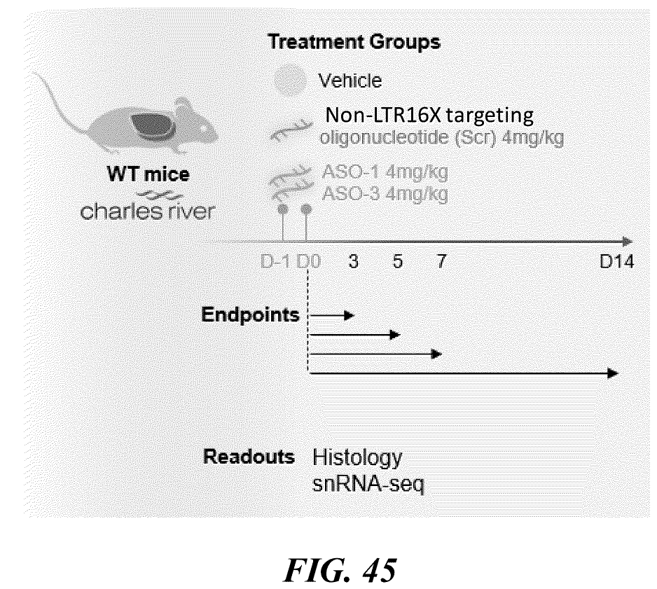

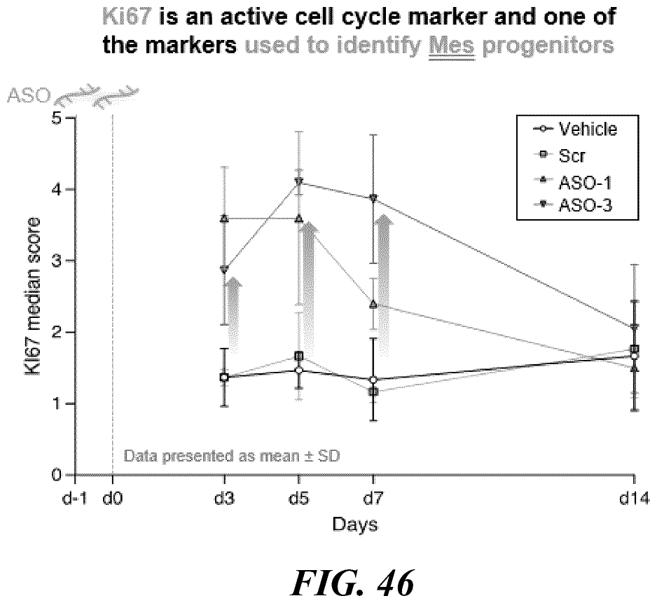

Separately, a time-course study in naive mice was performed to further infer mesenchymal progenitor dynamics and trajectory upon ASO treatment. As shown in FIG. 45, wild type C57BL/6J mice were oropharyngeally administered with either vehicle, ASO-scramble control (4 mg/mouse), or ASOs (ASO-1 or ASO-3 at 4 mg/mouse) at day-1 and day 0, and samples were taken at days 3, 5, 7 or 14 for analysis, where lungs were fixed for histopathology and was processed for immunostaining for Ki67, an active cell cycle marker and one of the markers used to identify mesenchymal progenitors. Thus, the number of Ki67 positive cells in mice were measured at days 3, 5, 7 and 14 after treatment with vehicle, ASO-scramble control, or ASOs. As shown in FIG. 46, there was increase in Ki67 positive cells in ASO-treated mice compared to vehicle or scramble control treated mice at days 3, 5, and 7 of treatment, suggesting emergence of mesenchymal progenitors. Moreover, the ASO-treated mice presented a localization of Ki67 positive cells at bronchiolar epithelium compared to mice treated with vehicle or scramble control. This readout suggests a potential fibroblast reprogramming into mesenchymal progenitors in the alveolar and bronchiolar epithelium. From days 7 to 14, however, there was a decrease in Ki67 positive cells, suggesting a potential differentiation of mesenchymal progenitors into a different cell type, such as AT2 cells.

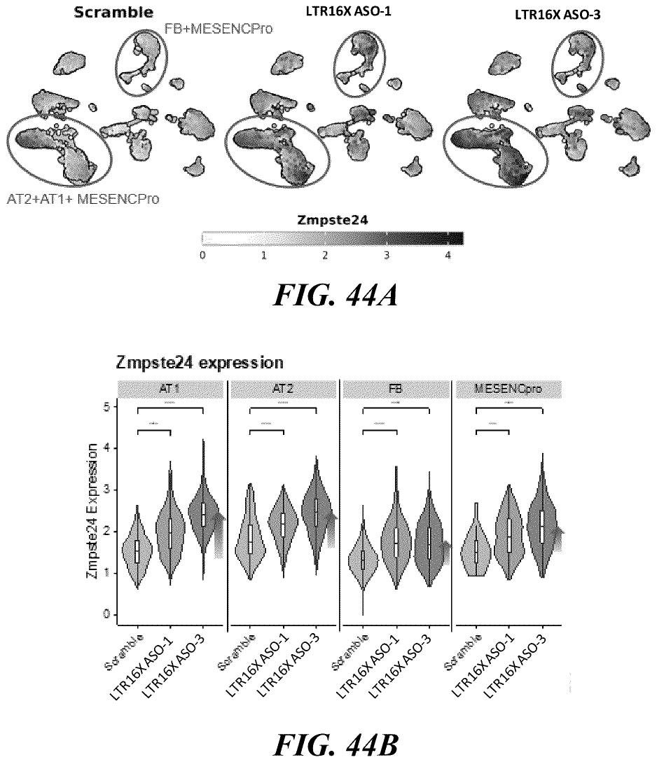

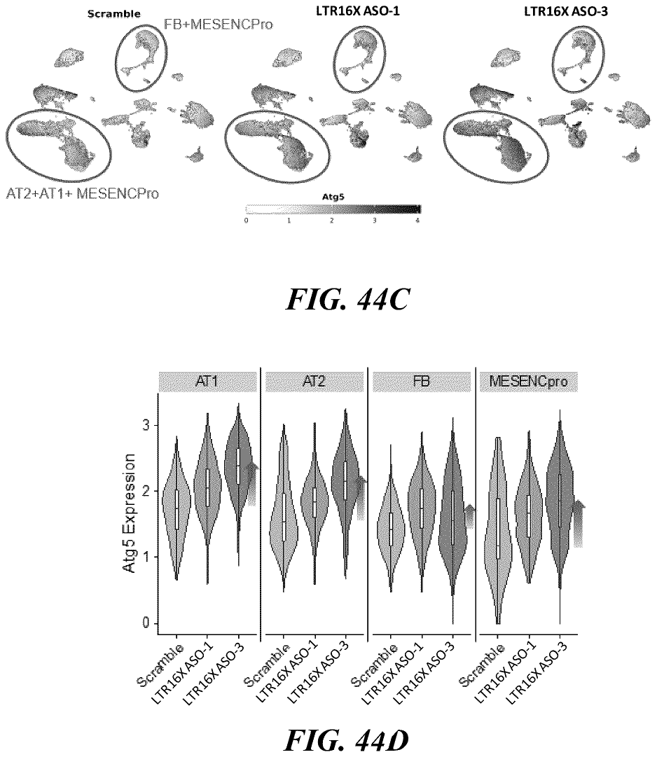

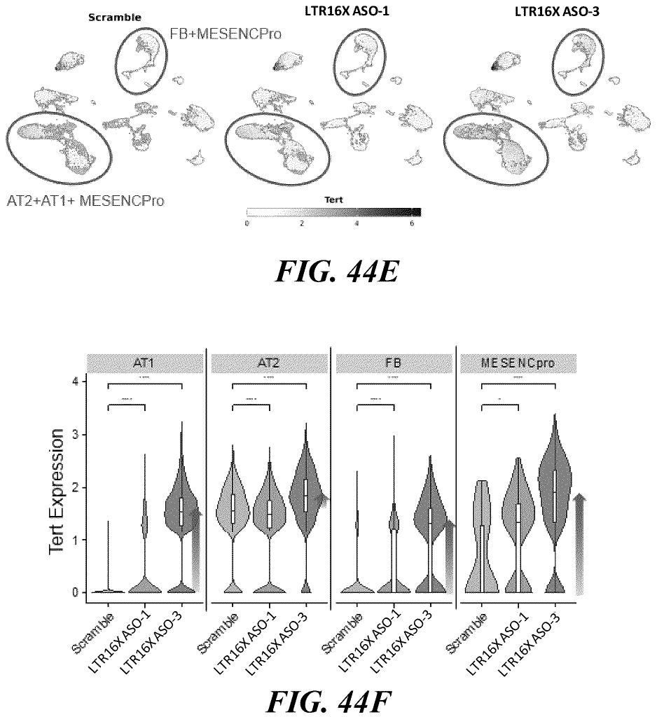

Furthermore, the expression of Zmpste24, a known proteostasis gene, was upregulated in AT1, AT2, fibroblasts and mesenchymal progenitor cells with treatment with ASO-1 or ASO-3, compared to treatment with Scramble control (FIG. 44A and FIG. 44B). Similarly, the expression of Atg5, which is associated with autophagy, was upregulated in AT1, AT2, fibroblasts and mesenchymal progenitor cells with treatment with ASO-1 or ASO-3, compared to treatment with Scramble control (FIG. 44C and FIG. 44D). The expression of Tert, which is associated with promoting telomerase, was also upregulated in AT1, AT2, fibroblasts and mesenchymal progenitor cells with treatment with ASO-1 or ASO-3, compared to treatment with Scramble control (FIG. 44E and FIG. 44F). Collectively, these data showed that treatment with ASOs affect hallmarks of rejuvenation by increasing proteostasis, increasing autophagy, and increasing telomerase, suggesting improved metabolic health in naive mice.

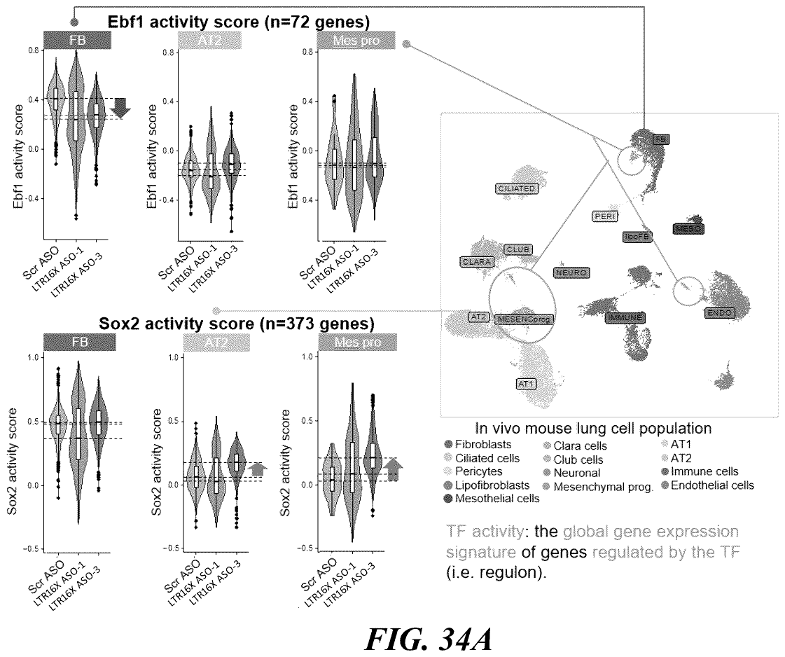

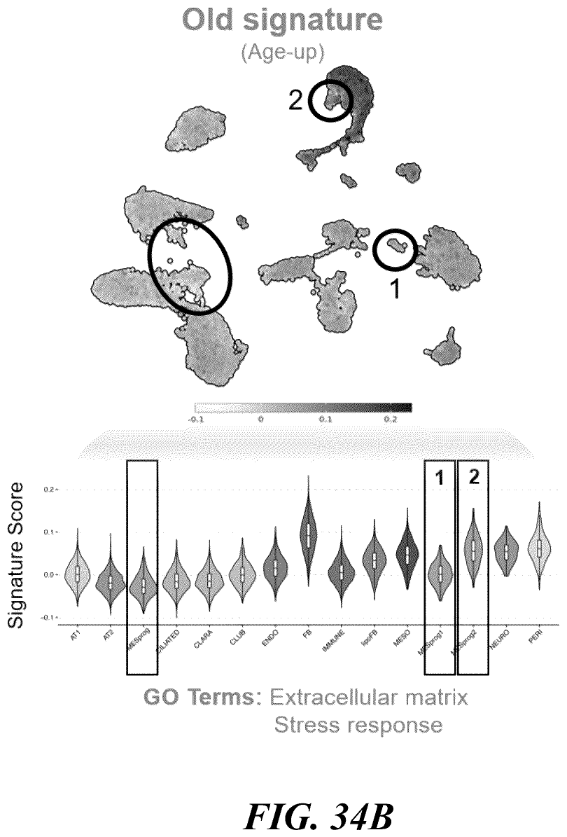

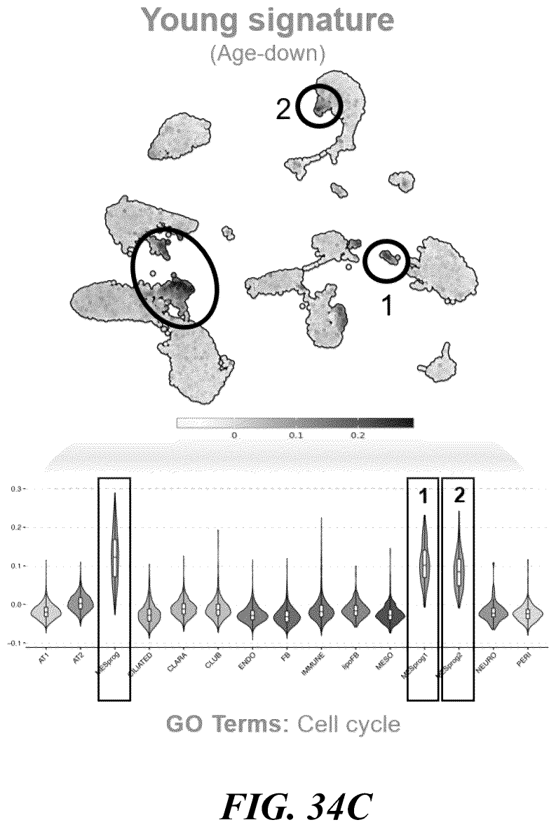

Next, transcription factor activity was analyzed using transcriptional regulatory networks (TRNs) as another approach to characterize relevant cell states at the single cell resolution upon ASO treatment. As shown in FIG. 34A, the activity of Ebf1, a cell fate-determining transcription factor, was specifically decreased in fibroblasts upon ASO-1 or ASO-3 treatment, which may allow for partial cellular reprogramming of fibroblasts to mesenchymal progenitors. The activity of Ebf1 was not affected in alveolar type II (AT2) and mesenchymal progenitors with ASO-1 or ASO-3 treatment. Furthermore, as shown in FIG. 34A, the activity of Sox2, a lung regeneration transcription factor, was increased in mesenchymal progenitor and AT2 cells, which may allow lung rejuvenation.

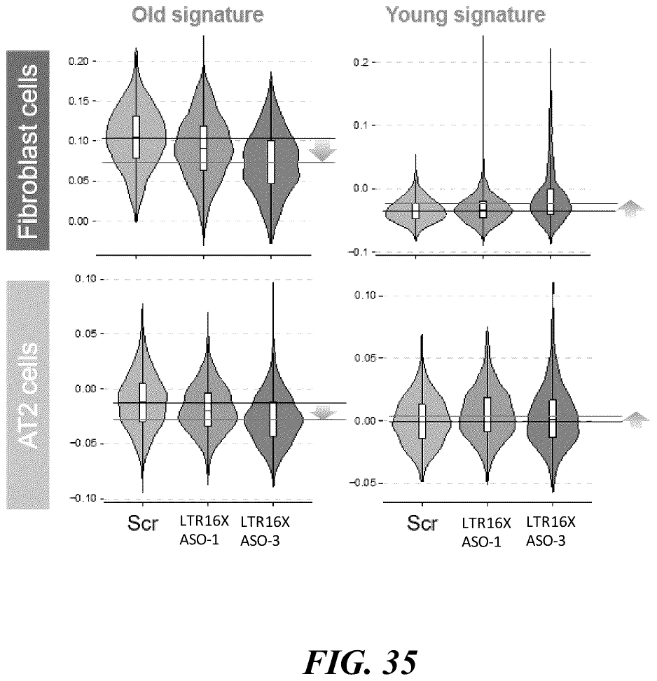

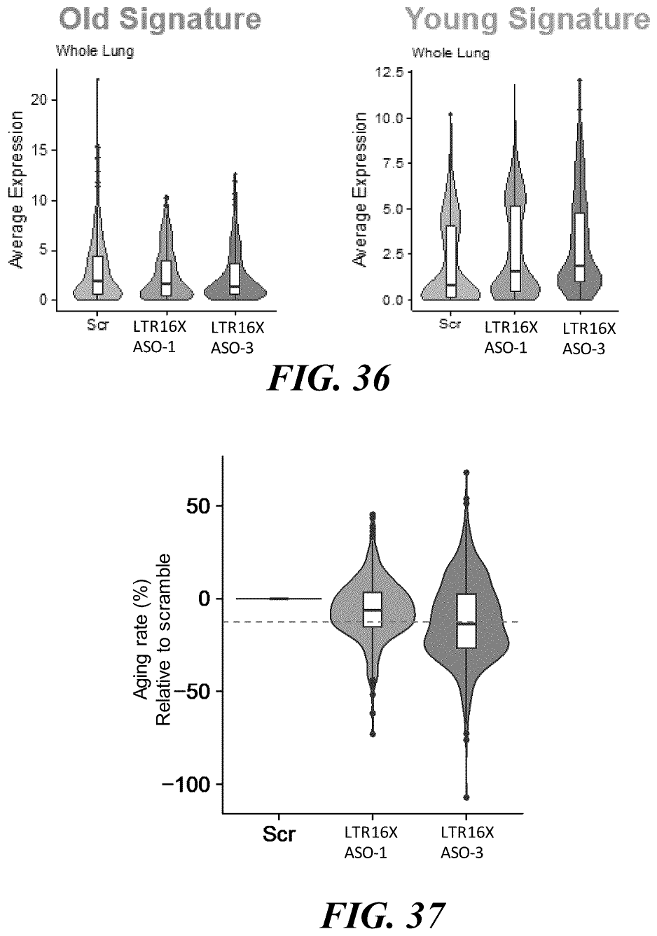

To investigate whether treatment of ASO-1 and ASO-3 can lead to transcriptomic rejuvenation in in vivo mice, the snRNA-seq data was further analyzed for old and young gene signature. The human-derived old and young gene signature described in Example 4 was converted to mouse ortholog genes. Then, the mouse old and young gene signature was applied to the in vivo mouse snRNA-seq data. As shown in FIG. 34B, there was minimal difference in old signature in ASOs treatment-specific mesenchymal progenitor cells compared to other cell types; however, as shown in FIG. 34C, there was increased young signature in ASOs treatment-specific mesenchymal progenitor cells compared to other cell types, suggesting transcriptomic rejuvenation of ASO-1 and ASO-3 reprogrammed cells in in vivo mice. Fibroblast and AT2 cell types from ASO-1 or ASO-3 treated mice lung exhibited reduced aging signature as well, as shown in FIG. 35. Likewise, there was a reduction in old signature expression and increase in young signature in whole lung of mice treated with ASO-1 or ASO-3 compared to scramble control (scr) (FIG. 36). The in vivo aging rate was also found to be reduced by about 10-20% in mice treated with ASO-1 or ASO-3 compared to mice treated with scramble control, as shown in FIG. 37.

### Example 7: Effect of ASO-1 and ASO-3 in In Vivo Mouse Fibrotic Disease Model

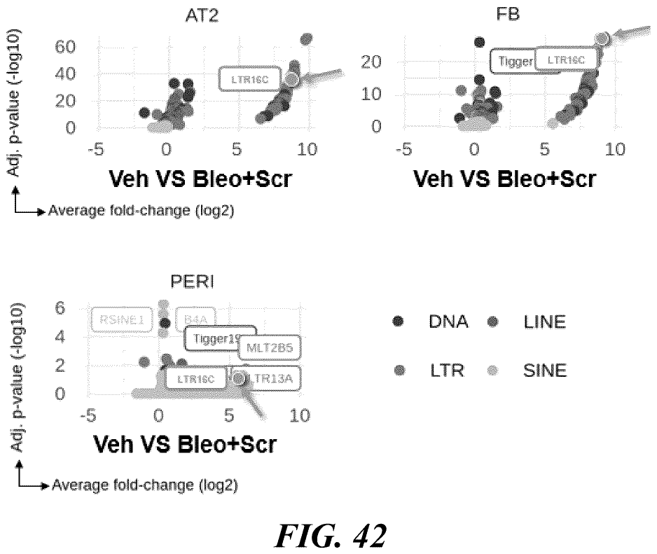

An in vivo mouse fibrotic disease model (e.g., bleomycin mouse model) was used to investigate the effect of ASOs in a diseased state. First, the expression of transposable elements (TE) was evaluated in bleomycin mouse model by snRNA-seq at day 21. The X axis of FIG. 42 is (log-transformed) fold change. The “0” denotes no fold change in either direction. If the displayed data point is to the left of 0, the corresponding subfamily is overall overexpressed in the vehicle animals, if it is to the right, the subfamily is instead overexpressed in animals treated with bleomycin and scramble, which means this subfamily is more disease-specific for the specified cell type. Y axis of FIG. 42 is adjusted p-value (i.e. a measure of significance).

As shown in FIG. 42, the analysis of snRNA-seq revealed that LTR16C subfamily was strongly upregulated, among other TEs, in bleomycin-treated AT2 and fibroblasts (FB), while LTR16C subfamily was not significantly upregulated in bleomycin-treated pericytes (Peri), suggesting that LTR16C elements are upregulated in disease relevant lung cell types (fibroblasts, AT2) in the bleomycin mouse model treated with scramble control compared to naive mice treated with vehicle control. Being upregulated in AT2 and FB indicates LTR16C subfamily is a desirable target in these fibrosis- and regeneration-relevant cell types. This suggested this TE subfamily can be used as a biomarker for pulmonary fibrosis (e.g., diagnosis or prognosis).

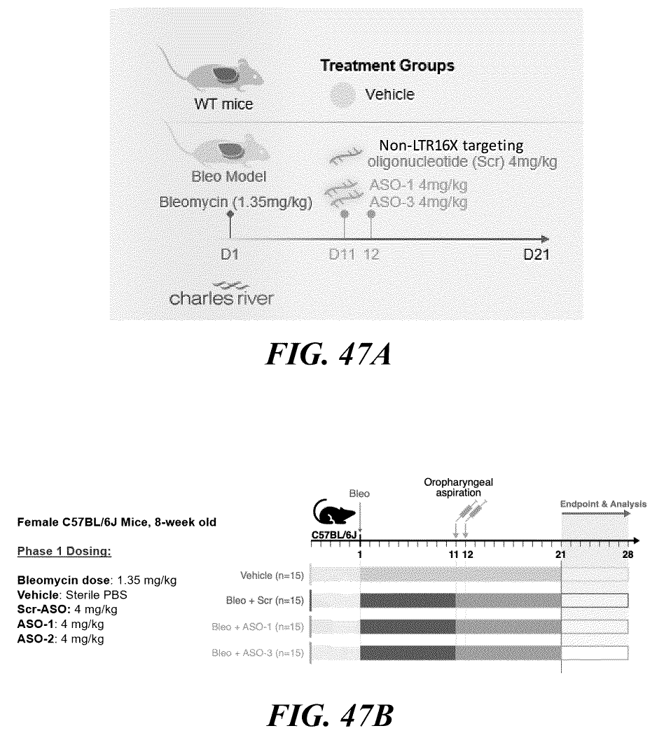

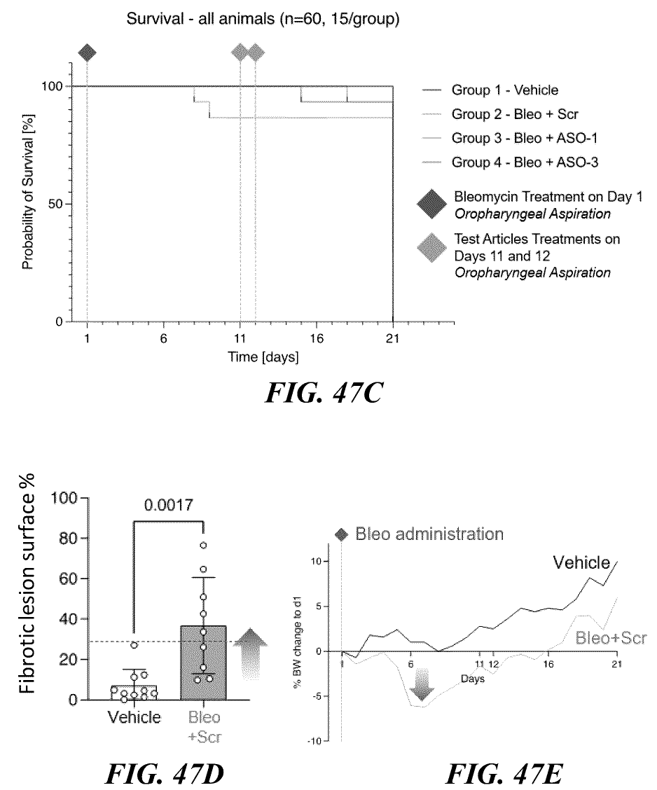

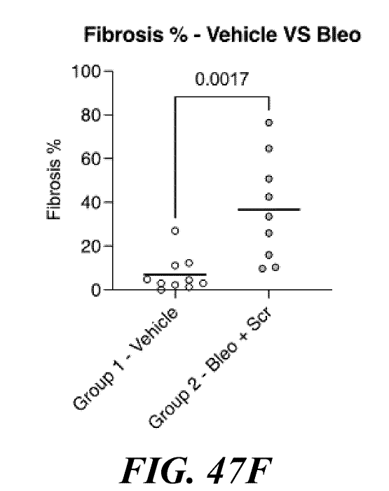

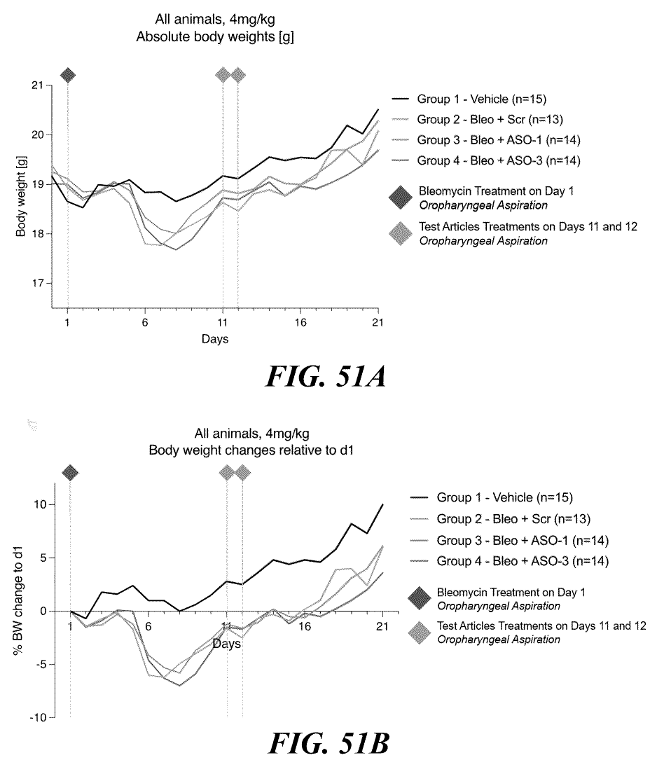

The bleomycin mouse model was also used to test the effect of ASO-1 and ASO-3 on cellular reprogramming and rejuvenation upon lung injury. As shown in FIG. 47A and FIG. 47B, C57BL/6 mice (8-week old) were treated with bleomycin (1.35 mg/kg) at day 1, and were oropharyngeally administered either scramble control (4 mg/kg), ASO-1 (4 mg/kg) or LTR16 ASO-3 (4 mg/kg) at days 11 and 12. Naive mice were treated with vehicle as a control. Downstream readouts were measured at day 21. The study was performed in two staggers, with a first set of n=5/group and a second set of n=10/group (separated by a week). The staggered approach allowed an examination on the tolerability of the treatment from a pilot group before treating a larger group. In the end, the two sets were found to be consistent and data were pooled together for further analysis. As shown in FIG. 47C, survival was stable and similar across different treatment groups. In addition, to confirm the effect of the bleomycin treatment and the bleomycin mouse model, the percentage of fibrotic lesion surface was measured. As shown in FIG. 47D, there was a statistically significant increase in fibrotic lesion surface (e.g., about 30-40%) in mice treated with bleomycin and scramble control compared to mice treated with vehicle, similar to published benchmarks of percent fibrosis of bleomycin mouse model. Similarly, as shown in FIG. 47F, there was 30-40% average fibrotic lesion surface, measured by quantifying picrosirius red (PSR) staining, in mice treated with bleomycin and scramble control compared to mice treated with vehicle. This result was similar to published benchmarks from bleomycin-treated mice. In addition, as shown in FIG. 47E, there was a transient drop in body weight (BW) (e.g., about 7%) observed at days 6-8 in mice treated with bleomycin and scramble control compared to mice treated with vehicle, as expected based on published benchmarks for body weight change in a bleomycin mouse model. As shown in FIG. 51A, mice treated with bleomycin and scramble control, mice treated with bleomycin and ASO-1, and mice treated with bleomycin and ASO-3 had similar absolute body weight loss over 21 days, with the peak bleomycin-related weight loss observed on days 7-8. Similarly, as shown in FIG. 51B, the relative weight change from day 1 to day 21 was consistent across the bleomycin treated mice groups, and peaked at day 7, verifying the bleomycin mice model.

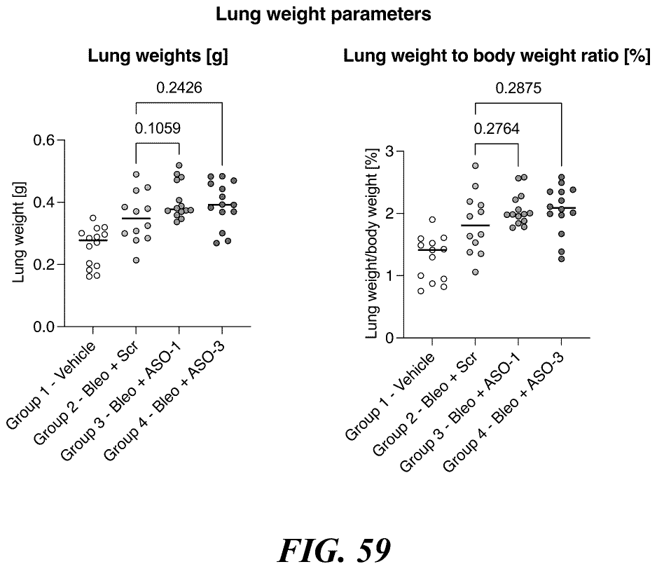

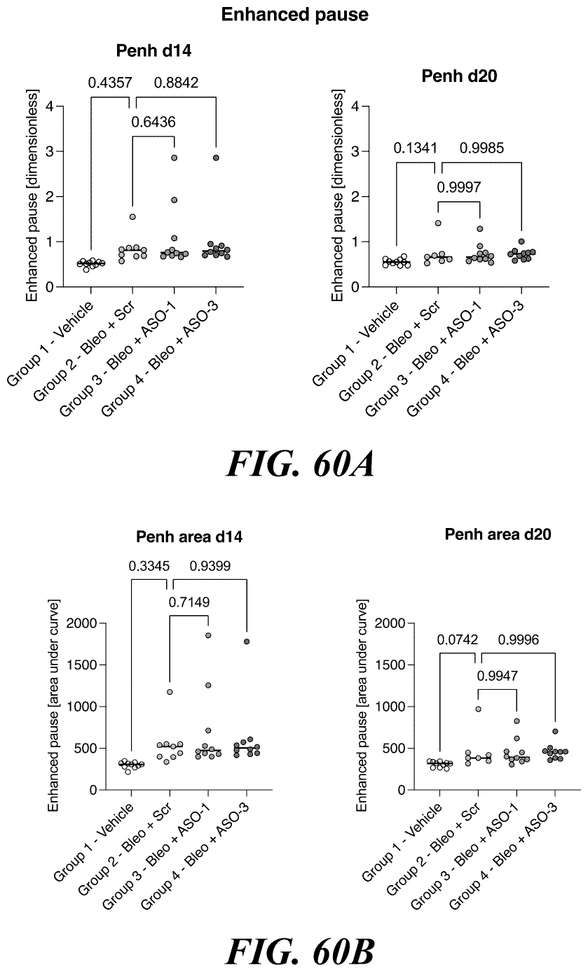

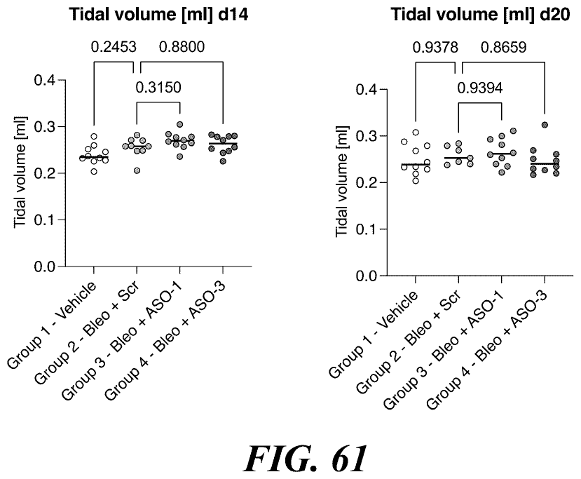

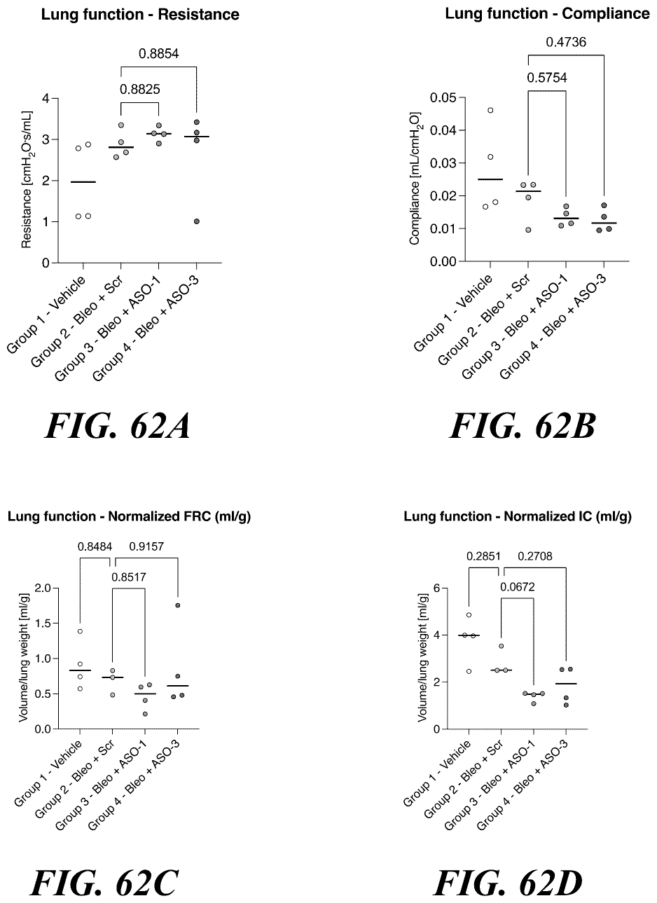

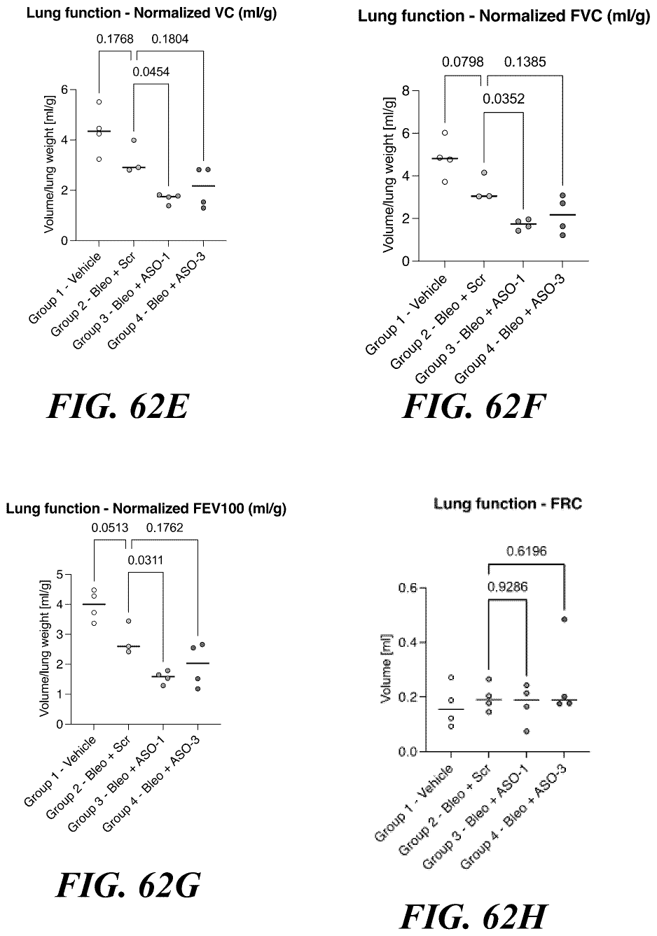

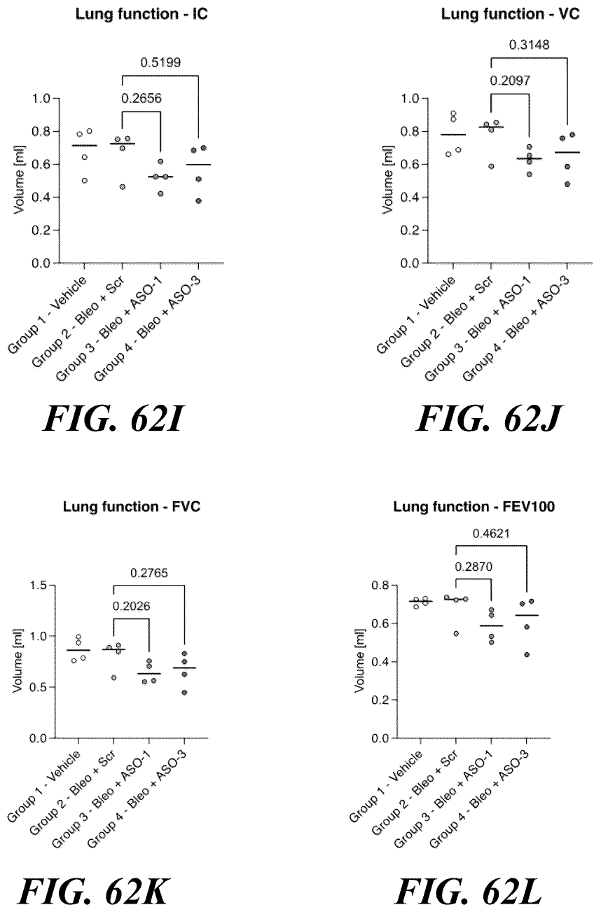

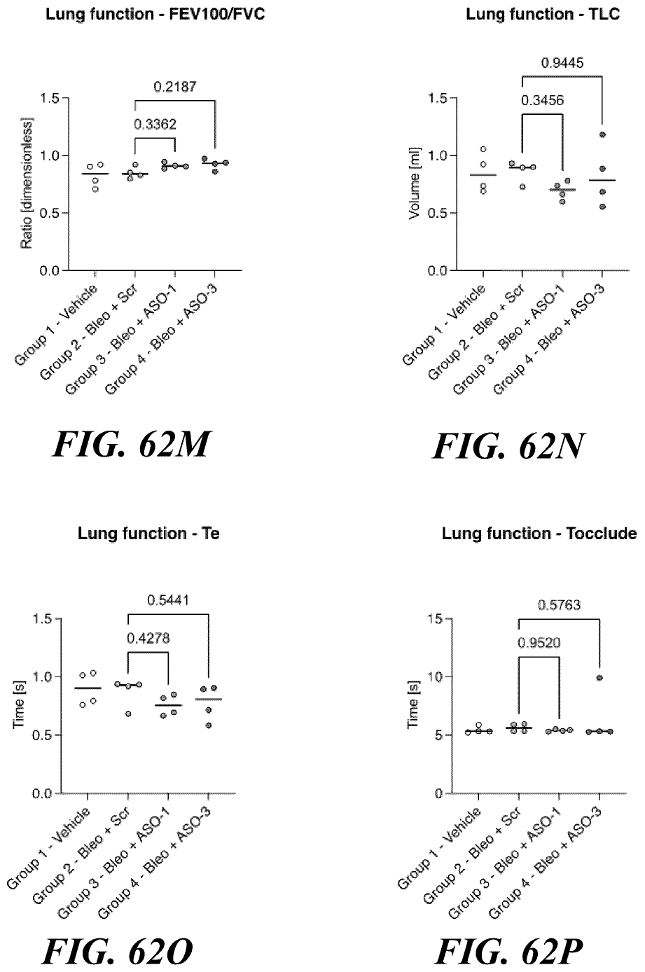

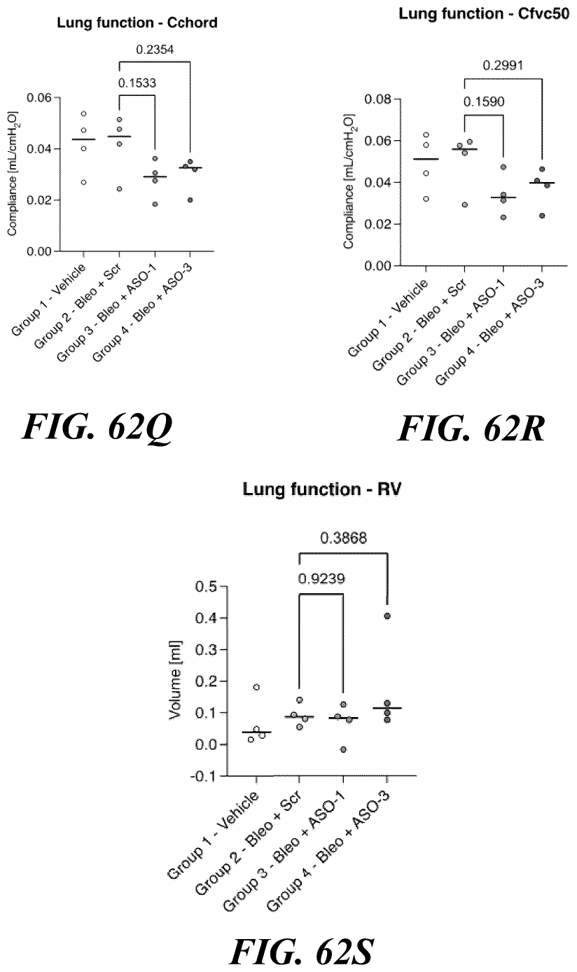

Further analysis was performed on the lungs of the naive mice treated with vehicle, and bleomycin mouse model treated with scramble control, ASO-1 or ASO-3. As shown in FIG. 59, the lung weight and lung weight to body weight ratio were measured. Functional lung capacity (enhanced pause (Penh)) was also measured by performing whole body plethysmography (WBP) to evaluate the changes in the shape of the airflow pattern entering and leaving. As shown in FIG. 60A and FIG. 60B, Penh after 14 days with treatment of bleomycin and scramble control, ASO-1, or ASO-3 was measured, either measured dimensionless or by the area under the curve. Similarly, Penh values measured dimensionless or by the area under the curve were measured at day 20 after the treatment. Tidal volume which is the volume of air moving in and out during one breathing motion was also measured by WBP at day 14 and day 20 (FIG. 61). In addition, lung resistance and lung compliance were measured by pulmonary function tests in naive mice treated with vehicle, and bleomycin mouse model treated with scramble control, ASO-1 or ASO-3 (FIG. 62A and FIG. 62B). Functional residual capacity (FRC), inspiratory capacity (IC), vital capacity (VC), forced vital capacity (FVC), and forced expiratory volume over 100 ms (FEV100) were measured and normalized by lung weight (62C-62G). Similarly, various other lung function parameters, such as total lung capacity (TLC), duration of expiration (Te), time the subject was occluded (Tocclude), chord compliance between 0-10 cm H2O [ml/cmH2O] (Cchord), compliance at 50% vital capacity [ml/cmH2O] (Cfvc50), and residual volume (RV), were measured. Quantification of a complete panel of lung function parameters can be found in FIGS. 62H-62S.

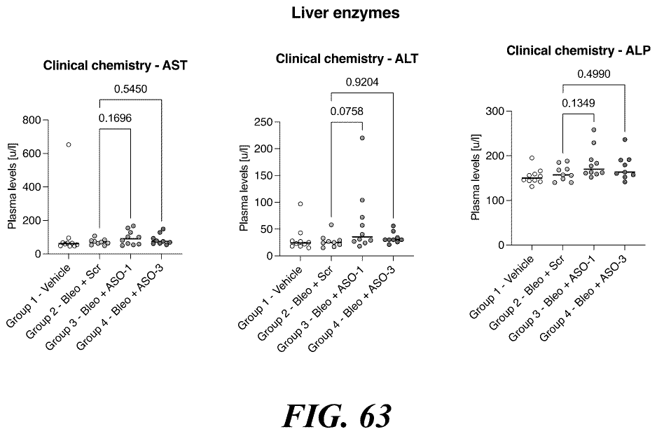

Furthermore, liver enzymes upon each treatment were evaluated. There was also no difference in levels of liver enzymes (aspartate aminotransferase (AST), alanine transaminase (ALT), alkaline phosphatase (ALP)) in plasma of bleomycin mice mouse model treated with ASO-1 or ASO-3 compared to bleomycin mice mouse model treated with scramble control, as shown in FIG. 63, suggesting tolerability of the ASOs.

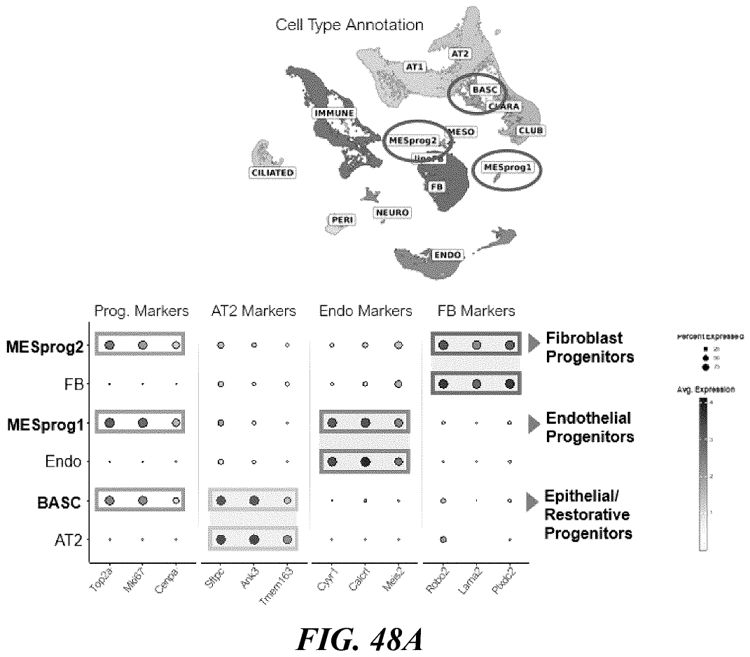

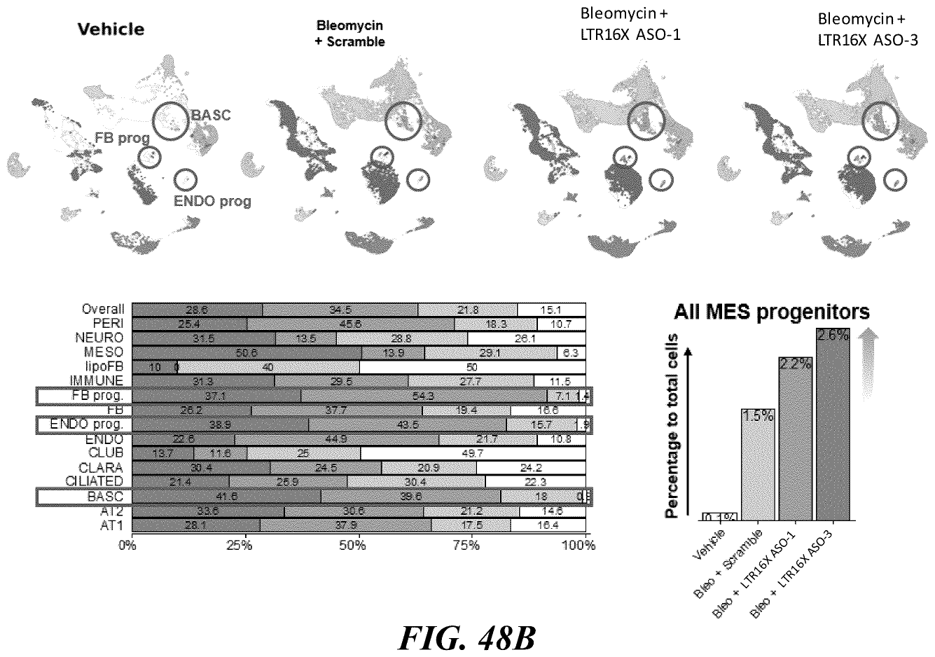

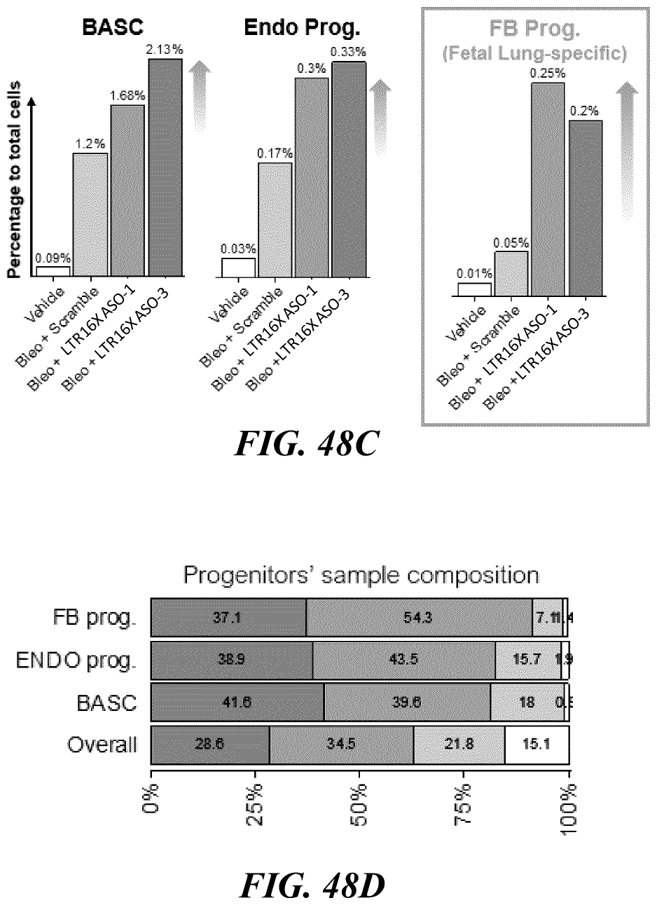

Once the bleomycin mice model was confirmed, snRNA-seq was performed with bleomycin mouse lungs harvested after treatment with scramble control, ASO-1 or ASO-3. Analysis of the snRNA-seq of bleomycin mouse model lungs treated with ASOs revealed the emergence of three different subtypes of mesenchymal progenitors (BASC, MESprog1, and MESprog2), as shown in FIG. 48A. BASC corresponds to epithelial progenitor cells. “MESprog1” is an internal label for endothelial progenitor cells. “MESprog2” is an internal label for fibroblast progenitor cells. The three subtypes of mesenchymal progenitors shared the same progenitor markers (e.g., Top2a, Mki67, Cenpa); however, they also had specific signatures, indicating that each type of mesenchymal progenitors are closer to certain cell types. For example, BASC was found to express AT2 marker genes and may represent a reparative cell type expanded upon injury. In addition, endothelial progenitors (MESprog1) were found to express endothelial markers, and fibroblast progenitors (MESprog2) were found to express fibroblast markers. As shown in FIGS. 48B-48D, there was an increase in the three subtypes of mesenchymal progenitors (BASC, MESprog1, and MESprog2) in bleomycin mouse lungs treated with ASOs compared to bleomycin mouse lungs treated with scramble control or naive mice treated with vehicle. In addition, the total mesenchymal progenitors were found to be increased in bleomycin mouse lungs treated with ASOs compared to bleomycin mouse lungs treated with scramble control or naive mice treated with vehicle, suggesting progenitor replenishment in ASO-treated bleomycin mice, which has potential to improve disease resolution.

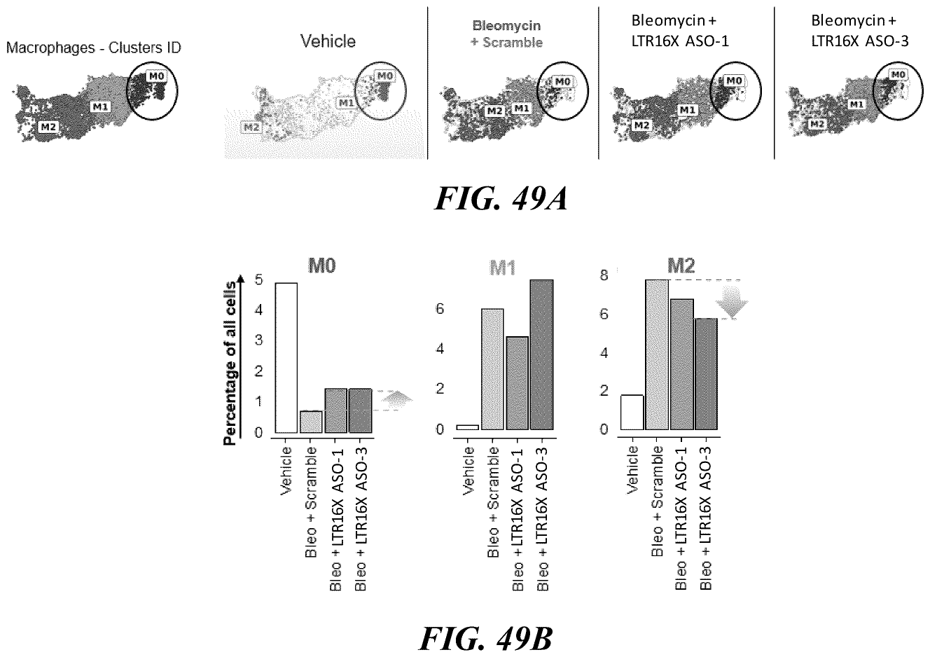

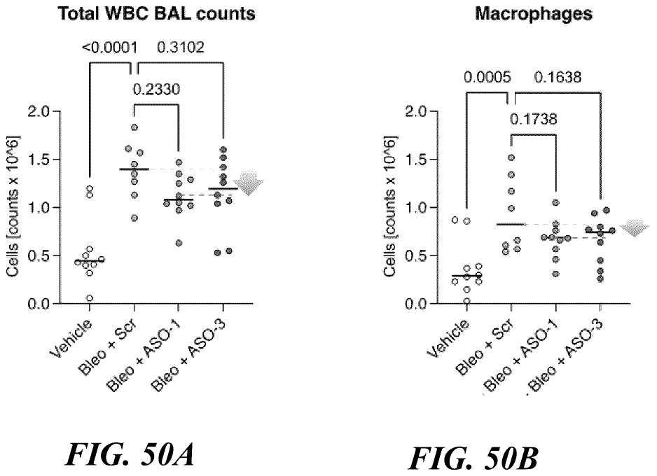

Age-related changes in the lung are associated with chronic inflammation and altered immune function. Repolarization of M1 and M2 macrophages to M0 macrophages, which exist in homeostasis, can help mitigate age-related inflammation and restore immune balance, thereby preserving lung function and reducing the risk of age-related diseases. Thus, whether treatment of ASOs affect macrophage population in lung tissue was explored. The snRNA-seq data was analyzed for macrophage population, and it was found that upon treatment with ASO-1 or ASO-3, in lung tissue of bleomycin treated mice, there was an increase in M0 macrophages and further decrease in profibrotic M2 macrophages (up to 25%) compared to bleomycin mouse model treated with scramble control, as shown in FIGS. 49A-49B. This data showed that treatment with ASOs can modulate macrophage activation, which is not only evidence that the ASO treatment effectively alleviated pulmonary fibrosis by reducing inflammation, but also a strong indication that the ASO treatment reduced age-related chronic inflammation. Furthermore, echoing the immune remodeling observed on M2 macrophages in tissue, the total white blood cells (WBC, leukocyte) and macrophage numbers in bronchioalveolar lavage (BAL) of ASO-1 or ASO-3 treated bleomycin mouse model decreased compared to scramble control treated bleomycin mouse model (FIGS. 50A-50B).

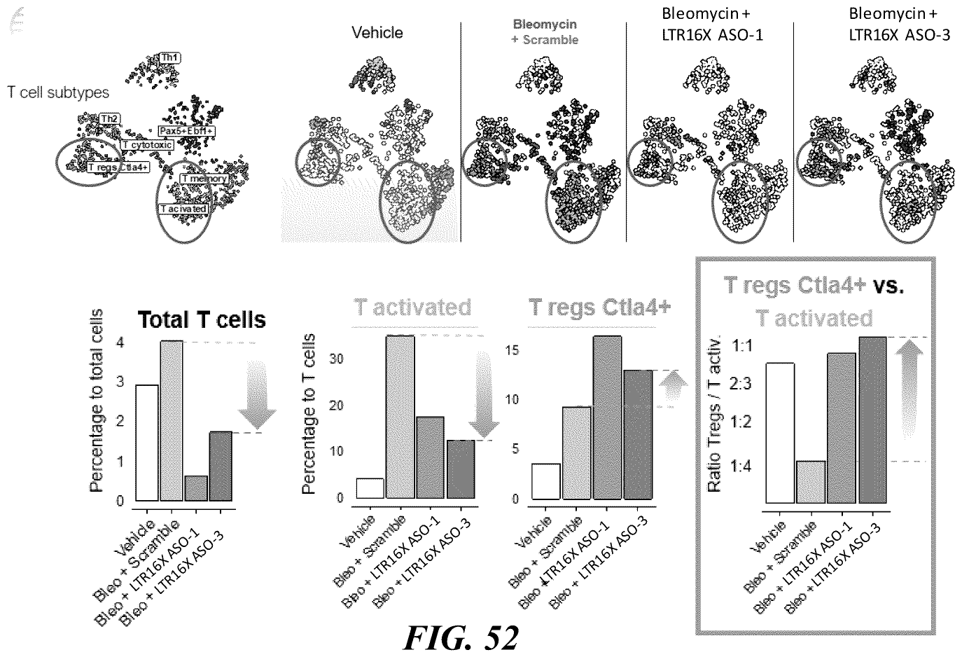

Aside from macrophages, age-related decline in tissue repair mechanism may be exacerbated by chronic inflammation mediated by activated T cells. Thus, the snRNA-seq data was also analyzed for T cell counts, activated T cells, and immunosuppressive Ctla4-expressing cells. As shown in FIG. 52, upon treatment with ASO-1 or ASO-3, lung tissue of bleomycin mice model had reduced total T cell counts and activated T cell counts, but increased immunosuppressive Ctla4-expressing cells compared to bleomycin mouse model treated with scramble control.

Collectively, these data suggest that the ASO treatment regulated macrophages and T cells toward an anti-inflammatory state, potentially halting chronic inflammation.

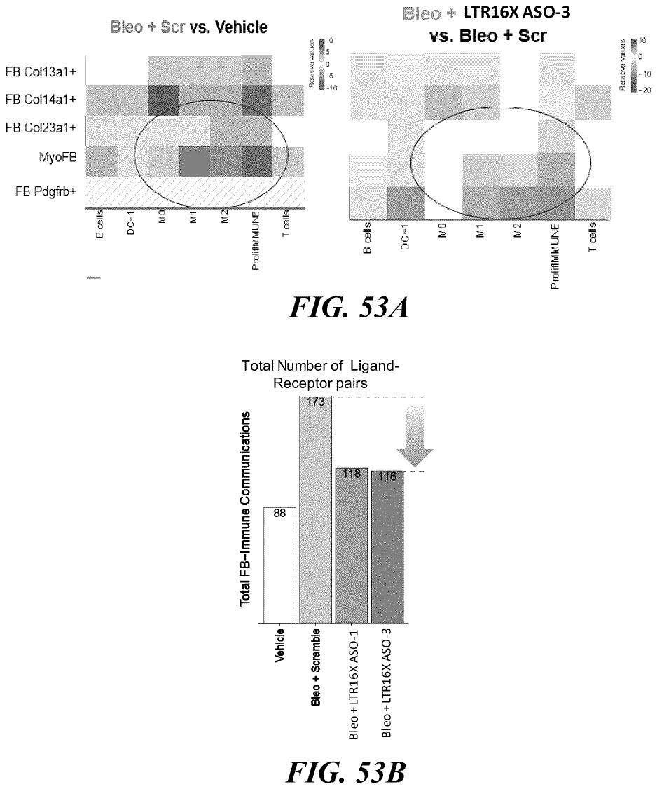

With modulation observed with immune cells upon treatment of ASOs in bleomycin mouse model, whether ASOs can affect the fibroblast and immune cell communication network was next investigated. Altered cell-cell communication is also a hallmark of aging. As shown in FIG. 53A, bleomycin-induced lung injury stimulated cell-cell communication between fibroblasts and immune cells (e.g., T cells, M0, M1, M2 macrophages) compared to mice treated with vehicle. Mice treated with bleomycin and ASO-3, however, presented reduced the cell-cell communication between fibroblasts and immune cell types. The total number of ligand-receptor pairs between fibroblast and immune cells was also decreased in mice treated with bleomycin and ASO-1 or ASO-3, compared to mice treated with bleomycin and scramble control (FIG. 53B), suggesting a restoration of normal cell-cell communication, which is a strong indication that the ASO treatment reduced age-related altered intercellular communication, but also not only evidence that the ASO treatment effectively alleviated pulmonary fibrosis by reducing fibroblast and immune cell communication.

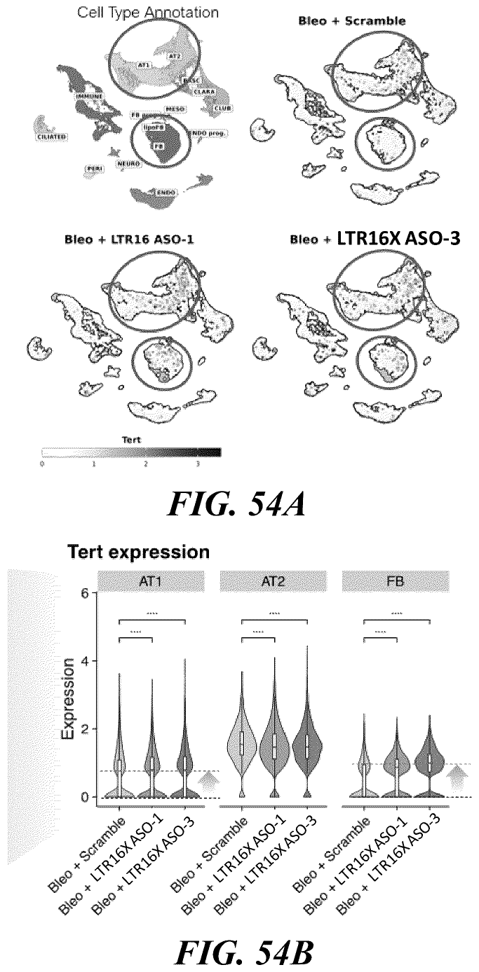

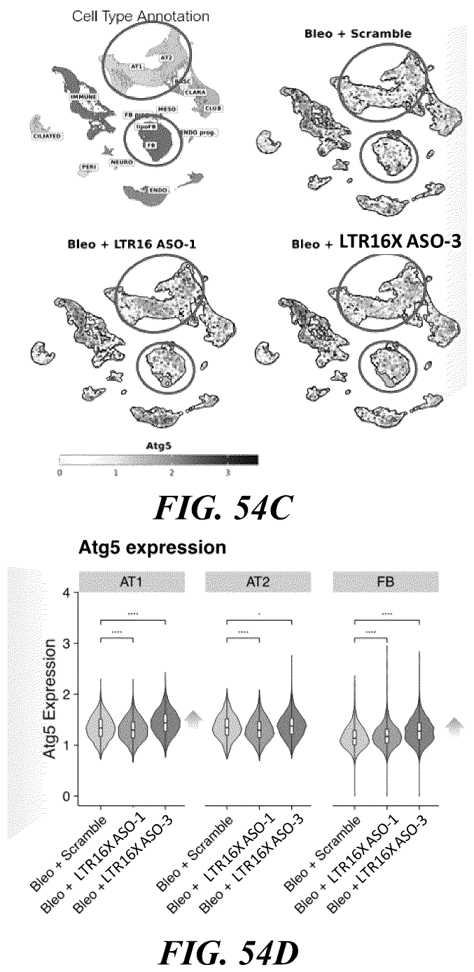

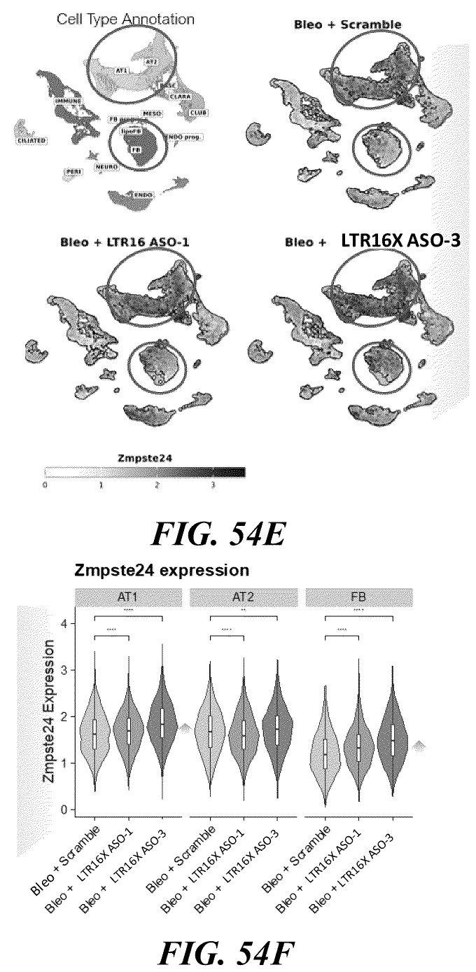

Next, the snRNA-seq was analyzed for expression of genes associated with aging. As shown in FIG. 54A and FIG. 54B, the expression of Tert, which encodes the main subunit of telomerase, was found to be upregulated in AT1 and fibroblasts in the bleomycin mouse model treated with ASO-1 or ASO-3, compared to treatment with Scramble control. The increased telomerase expression in specific lung cell types in bleomycin mice treated with ASOs indicated the potential for telomere extension, slowing down progression of pulmonary fibrosis, and/or reducing severity of progression. The expression of Atg5, a macroautophagy gene, was also found to be upregulated in AT1 and fibroblasts in bleomycin mouse model treated with ASO-1 or ASO-3, compared to treatment with Scramble control (FIG. 54C and FIG. 54D). The increase in Atg5 expression in specific lung cell types suggested enhanced metabolism and potential extended lifespan. Similarly, the expression of Zmpste24, a known proteostasis gene, was found to be upregulated in AT1 and fibroblasts in the bleomycin mouse model treated with ASO-1 or ASO-3, compared to treatment with Scramble control (FIG. 54E and FIG. 54F). The increase in Atg5 expression in specific lung cell types suggested increase in proteostasis, potentially leading to reduced gaining and amelioration of pulmonary fibrosis.

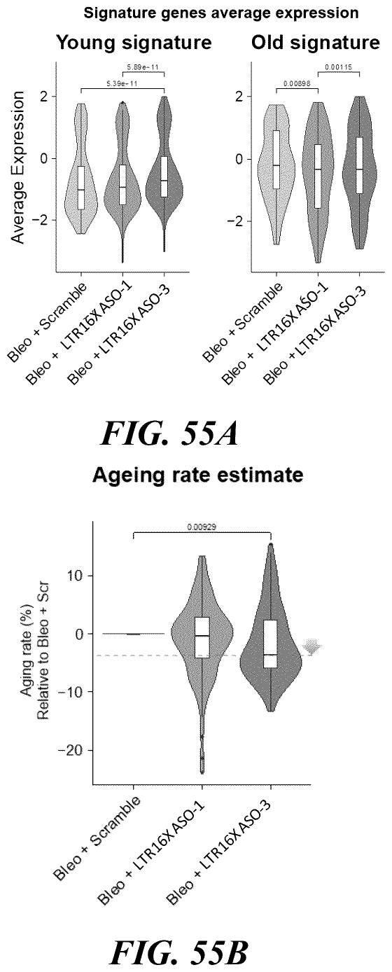

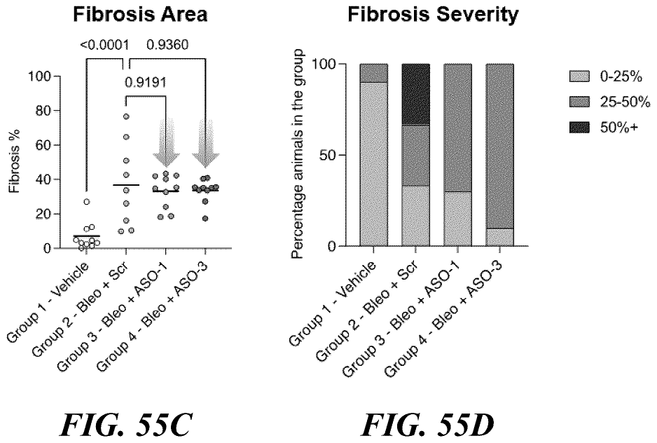

SnRNA-seq data were further profiled for old and young gene signature to see whether treatment with ASOs can induce transcriptional lung rejuvenation in the bleomycin mouse model. As shown in FIG. 55A, there was increased expression of young signature and decreased old signature in bleomycin mouse model treated with ASO-1 or ASO-3, compared to scramble control, suggesting transcriptional lung rejuvenation in bleomycin mouse model with ASO treatment. In additional, with treatment of bleomycin and ASO-3, there was a negative aging rate of the whole lung compared to treatment with bleomycin and scramble control (see FIG. 55B). These data suggested that treatment of ASOs induced partial cellular reprogramming that reversed the overall lung transcriptomic clock in a bleomycin mouse model.

Histopathological quantification of picrosirius red-stained lung sections also indicated a reduction in fibrosis area and fibrosis severity in bleomycin mouse model treated with ASO-1 or ASO-3, compared to the bleomycin mouse model treated with scramble control, as shown in FIG. 55C and FIG. 55D. Mice with more than 50% fibrosis were not present in the group of bleomycin mice treated with ASO-1 or ASO-3.

Collectively, these data showed that treatment with ASOs in the bleomycin mouse model promoted lung rejuvenation.

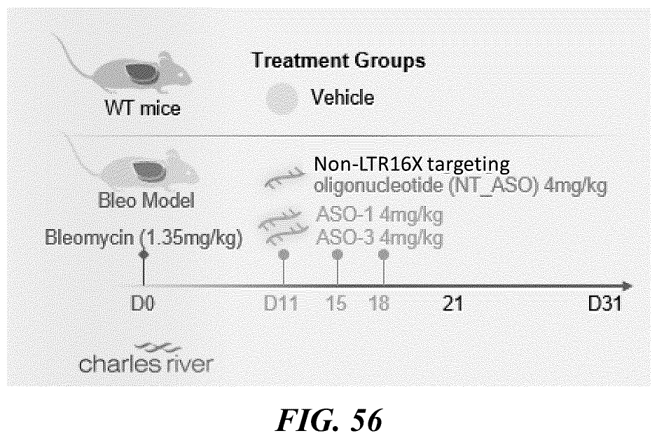

In addition, as shown in FIG. 56, C57BL/6 mice (8-10 weeks of age) are treated with bleomycin (1.35 mg/kg) at day 0, and treated with scramble control (NT_ASO, 4 mg/kg), ASO-1 (4 mg/kg), or ASO-3 (4 mg/kg) at days 11, 15 and 18. Naive mice are treated with vehicle as a control. The lungs of the mice are harvested at day 31 and various analyses are conducted to investigate changes in partial cellular reprogramming and rejuvenation in lung disease resolution as done with lungs harvested at day 21. Experiments as described in Example 6 are also conducted. Transcriptomic rejuvenation, tissue inflammation and fibrosis severity by histopathology analysis are measured. Further snRNA-seq and/or bulk RNA-seq of the tissues harvested is conducted to assess various hallmarks of aging, such as chronic inflammation, altered intercellular communication, emergence and expansion of progenitors, lung transcriptomic aging clock, telomere attrition, loss of proteostasis, and disabled macroautophagy. Different multiomics experiments, such as snATAC-seq, Micro-C, and Cut&Run, are performed to assess epigenetic alterations. Clinical chemistry markers and metabolomics, measured by NMR and LC-MS on small molecules and/or lipids, are assessed to understand the effect of the ASOs on aging.

### Example 8: Effect of ASO-1 and ASO-3 to In Vivo Mouse Aged Model

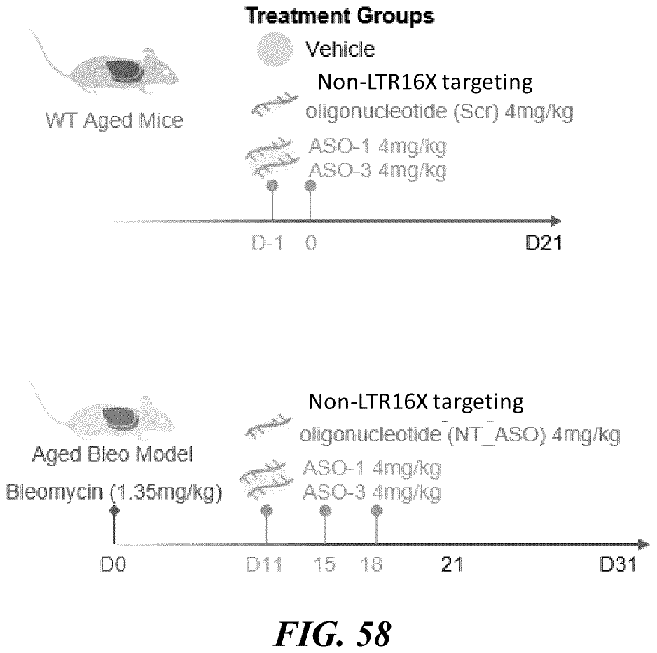

As shown in FIG. 58, aged naive mice are treated with vehicle, scramble control, ASO-1, or ASO-3 (4 mg/kg or various doses that are appropriate) at day-1 and/or day 0. Lungs are harvested for downstream analysis at e.g., day 21 or a day that is appropriate for harvest. Similarly, as shown in FIG. 58, aged mice are treated with bleomycin at day 0, and treated with scramble control, ASO-1, or ASO-3 (e.g, 4 mg/kg or various doses that are appropriate) at e.g., days 10, 11, 12, 15, 18 or a day that is appropriate for ASO treatments after the bleomycin model is established. Lungs are harvested for downstream analysis at e.g., day 31 or a day that is appropriate for harvest. Route of administration for ASOs includes but is not limited to intratracheally, intranasally, or other methods of direct instillation to the lungs. Bleomycin instillation includes but is not limited to intratracheal, intralobar, subcutaneous etc. Downstream analyses described in Example 6 and Example 7 are performed on the harvested lungs to investigate lung rejuvenation, change in fibrosis, and cellular reprogramming (e.g., emergence of mesenchymal progenitor cells) in in vivo aged model and in disease model. snRNA-seq and bulk RNA-seq of the tissues harvested are conducted to assess various hallmarks of aging, such as chronic inflammation, altered intercellular communication, emergence and expansion of progenitors, lung transcriptomic aging clock, telomere attrition, loss of proteostasis, and disabled macroautophagy. Genomic DNA is extracted. DNA methylation is profiled (e.g., WGBS, RRBS, MeDIP, Illumina methylation array, duet evoC kit, etc.) to assess the DNA methylation epigenetic clocks (e.g., GrimAge, Horvath) in animal tissues or peripheral blood. Different multiomic experiments, such as snATAC-seq, Micro-C, and Cut&Run, are performed to assess epigenetic alterations. Clinical chemistry markers and metabolomic readouts or metabolomics analyses, measured by NMR and LC-MS on small molecules and/or lipids, are assessed to understand the effect of the LTR16X ASOs on aging and on disease resolution in aged organisms. Blood, plasma, PBMCs are collected. All organs are collected and snap-frozen. Livers are fixed and blocked. Besides the rejuvenation readouts mentioned in the above examples, other exemplary readouts are measured, such as, Terc gene for telomeres; Lmna nuclear lamina gene; Atg7 for autophagy; Lamp2 for proteostasis; Sirt1, Sirt6 and Sirt7 for histone modifiers; and the PI3K-AKT and the Ras-MEK-ERK pathways for nutrient sensing.

Ex vivo experiments are also performed. A human donor precision cut lung slices (PCLS) model is used to validate transposable element expression and fibrotic induction by bulk RNA-seq. The fibrotic PCLS model is treated with LTR16X ASOs (e.g., ASO-1, ASO-3) and fibrotic assays, bulk RNA-seq, snRNA-seq etc. are performed to investigate the effect of LTR16X ASO treatment ex vivo.

### Example 9: Transposable Elements or Transposable Element Transcripts Used as Biomarkers

As shown in the above examples (e.g., FIGS. 1J-1M, 38A, 38B, and 42), LTR16C is strongly associated with pulmonary fibrosis (e.g., IPF). Therefore, it can serve as a biomarker, along with others (e.g., genes identified as IPF gene signature listed in Table 3 and genes identified as Bleomycin/MLF signature listed in Table 5).

A lung biopsy, a skin biopsy, blood, plasma, PBMCs, a sample of connective tissue, a sample of cartilage, mesenchymal cells, or any other forms of liquid biopsy are collected from a subject. Some cell types may be specifically isolated or FACS-sorted with appropriate methods.

RNA extraction, qPCR, and bulk RNA sequencing are performed on the whole biopsy or on specific cell types. Single cell or single nucleus RNA sequencing are performed. Genomic DNA extraction is performed. DNA sequencing is performed. DNA methylation profiling (e.g., WGBS, RRBS, MeDIP, Illumina methylation array, duet evoC kit, etc.) is performed. sc- or snATAC-seq or bulk ATAC-seq is performed. DNase I assay for accessibility is performed. CUT&RUN or ChIP-seq for specific histone marks (e.g., H3K27ac, H4K16ac, H3K9me3, H3K27me3, H3K4me1, H3K4me3, etc.) is performed.

Bulk or sc/snRNA-seq expression of transposable elements is assessed, both at single locus level and at subfamily level. Transposable element DNA methylation levels (for example, average % methylated CpG along the transposable element locus or the methylation status of specific CpGs within the transposable element) are assessed. Accessibility of transposable elements is quantified (single locus or subfamily) through sc/snATAC or bulk ATAC-seq or DNase I assay. Presence of individual or combinations of histone marks on specific loci or subfamilies of transposable elements is assessed. In addition, certain transposable elements encode detectable proteins, which can be used as biomarkers as well. Therefore, detection of transposable element-derived proteins using known technologies is also performed.

The above parameters can be assessed for a single transposable element locus, a set of transposable elements loci (e.g., a set of transposable element loci within the same subfamily such as but not limited to the ones listed in Table 2 or for a set of transposable element loci across different subfamilies such as but not limited to the ones listed in Table 1).

It shall be understood that different aspects of the disclosure can be appreciated individually, collectively, or in combination with each other. Various aspects of the disclosure described herein may be applied to any of the particular applications disclosed herein. The compositions of matter disclosed herein in the composition section of the present disclosure may be utilized in the method section including methods of use and production disclosed herein, or vice versa.

While preferred instances of the present disclosure have been shown and described herein, it will be obvious to those skilled in the art that such instances are provided by way of example only. It is not intended that the disclosure be limited by the specific examples provided within the specification. While the disclosure has been described with reference to the aforementioned specification, the descriptions and illustrations of the instances herein are not meant to be construed in a limiting sense. Numerous variations, changes, and substitutions will now occur to those skilled in the art without departing from the disclosure. Furthermore, it shall be understood that all aspects of the disclosure are not limited to the specific depictions, configurations or relative proportions set forth herein which depend upon a variety of conditions and variables. It should be understood that various alternatives to the instances of the disclosure described herein may be employed in practicing the disclosure. It is therefore contemplated that the disclosure shall also cover any such alternatives, modifications, variations or equivalents. It is intended that the following claims define the scope of the disclosure and that methods and structures within the scope of these claims and their equivalents be covered thereby.

### List of Exemplary Embodiments

Embodiment 1. A modulator of a nucleic acid derived from a transposable element, wherein the modulator induces or facilitates a change of a state of a cell from a first cellular state to a second cellular state.

Embodiment 2. The modulator of embodiment 1, wherein the nucleic acid is a transposable element or a fragment thereof.

Embodiment 3. The modulator of embodiment 1, wherein the nucleic acid is a transposable element transcript, wherein the transposable element transcript is transcribed from the transposable element.

Embodiment 4. The modulator of any one of embodiments 1-3, wherein the first cellular state or the second cellular state comprises an extracellular-cellular-matrix synthesizing state, a cytokine-secreting state, an inflammatory state, a cell dividing state, a cell pausing state.

Embodiment 5. The modulator of embodiment 4, wherein the first cellular state is the extracellular-cellular-matrix synthesizing state.

Embodiment 6. The modulator of embodiment 4 or 5, wherein the second cellular state is the inflammatory state.

Embodiment 7. The modulator of any one of embodiments 3-6, wherein the transposable element transcript modulates a functional RNA.

Embodiment 8. The modulator of embodiment 7, wherein the functional RNA comprises a messenger RNA (mRNA), a long noncoding RNA (lncRNA), a circular RNA, a small nuclear RNAs (snRNAs), a small nucleolar RNAs (snoRNAs), a microRNA, an X-inactive specific transcript (xist) RNA, or a combination thereof.

Embodiment 9. The modulator of embodiment 8, wherein the functional RNA comprises the lncRNA.

Embodiment 10. The modulator of embodiment 9, wherein the transposable element transcript and the lncRNA are transcribed from the same strand.

Embodiment 11. The modulator of embodiment 9, wherein the transposable element transcript and the lncRNA are transcribed from a different strand.

Embodiment 12. The modulator of any one of the preceding embodiments, wherein the transposable element or the transposable element transcript is associated with pulmonary fibrosis.

Embodiment 13. The modulator of any one of embodiments 3-12, wherein an RNA expression level of the transposable element transcript is altered in a cell affected by pulmonary fibrosis compared to a healthy cell.

Embodiment 14. The modulator of embodiment 13, wherein the RNA expression level of the transposable element transcript is increased in a cell affected by pulmonary fibrosis compared to the healthy cell.

Embodiment 15. The modulator of any one of the preceding embodiments, wherein the transposable element is bound by or bears a binding motif being bound by a pulmonary-fibrosis-related transcription factor.

Embodiment 16. The modulator of embodiment 15, wherein the pulmonary-fibrosis-related transcription factor comprises friend leukemia integration 1 transcription factor (FLI1), interferon regulatory factor 3 (IRF3), interferon regulatory factor 4 (IRF4), (ETS like-1 protein (ELK1), activator protein 1 (AP-1), C-terminal binding protein 2 (CTBP2), Spi-1 Proto-Oncogene (SPI1/PU.1), transcription factor 7 (TCF7), transcription factor 7-like 1 (TCF7L1), transcription factor 7-like 2 (TCF7L2), transcription Factor 21 (TCF21), CCAAT enhancer binding protein beta (CEBPB), forkhead box protein A1 (FOXA1), forkhead box protein A2 (FOXA2), forkhead box protein 04 (FOXO4), forkhead box protein L1 (FOXL1), forkhead box protein L2 (FOXL2), forkhead Box M1 (FOXM1), T-box transcription factor 4 (TBX4), T-box transcription factor 5 (TBX5), odd-skipped related 1 (OSR1), early B-cell factor 1 (EBF1), early B-cell factor 2 (EBF2), early B-cell factor 3 (EBF3), twist-related protein 1 (TWIST1), SMAD family member 2 (SMAD2), SMAD family member 3 (SMAD3), nuclear factor-1 (NF1), NK6 homeobox 1 (NKX6-1), NK2 homeobox 1 (NKX2.1), RUNX family transcription factor 2 (RUNX2), POU class 2 homeobox associating factor 1 (POU2AF1), high mobility group AT-hook 2 (HMGA2), lymphoid enhancer-binding factor 1 (LEF1), paired related homeobox 1 (PRRX1), tumor protein p63 (TP63), forkhead box protein P1 (FOXP1), Meis homeobox 1 (MEIS1), TGFB induced factor homeobox 1 (TGIF1), paired related homeobox 2 (PRRX2), TEA domain family member 1 (TEAD1), TEA domain family member 2 (TEAD2), TEA domain family member 3 (TEAD3), TEA domain family member 4 (TEAD4), signal transducer and activator of transcription 1 (STAT1), signal transducer and activator of transcription 3 (STAT3), signal transducer and activator of transcription 6 (STAT6), nuclear factor-kappa B1 (NFKB1), nuclear factor-kappa B2 (NFKB2), AT-rich interactive domain-containing protein 3A (ARID3A), forkhead box protein S1 (FOXS1), forkhead box protein J2 (FOXJ2), forkhead box protein I1 (FOXI1), forkhead box protein F1 (FOXF1), forkhead box protein E1 (FOXE1), forkhead box protein A3 (FOXA3), forkhead box protein D3 (FOXD3), MYC-Associated Zinc Finger Protein (MAZ), Zinc Finger Protein X-Linked (ZFX), Zinc Finger Protein Y-Linked (ZFY), transcription factor 12 (TCF12), transcription factor AP-2 gamma (TFAP2C), CCCTC-Binding Factor (CTCF), CCCTC-Binding Factor Like (CTCFL or BORIS), Myogenin (MYOG), Nuclear transcription factor Y subunit alpha (NFYA), Nuclear transcription factor Y subunit beta (NFYB), Nuclear transcription factor Y subunit gamma (NFYC), Upstream stimulatory factor 1 (USF1), Upstream stimulatory factor 2 (USF2), Jun Proto-Oncogene/AP-1 Transcription Factor Subunit (JUN), JunD Proto-Oncogene/AP-1 Transcription Factor Subunit (JUND), Fos Proto-Oncogene/AP-1 Transcription Factor Subunit (FOS), FOS Like 1/AP-1 Transcription Factor Subunit (FOSL1), MYC Associated Factor X (MAX), MAF BZIP Transcription Factor F (MAFF), or a combination thereof.

Embodiment 17. The modulator of any one of the preceding embodiments, wherein the transposable element transcript is bound by or targeted by an RNA-binding protein.

Embodiment 18. The modulator of embodiment 17, wherein the RNA-binding protein comprises of DDX3, RBM3, HuR, or a combination thereof.

Embodiment 19. The modulator of any one of the preceding embodiments, wherein the transposable element comprises an epigenetic modification or the transposable element transcript comprises an epitranscriptomic modification.

Embodiment 20. The modulator of embodiment 19, wherein the epigenetic modification comprises H3K27Ac, H3K4me3, H3K4me1, H4K16ac, H3K27me3, H3K79me2, H3K36me3, H2AFZ, H3K9ac, H3K4me2, H4K20me1, H2BK120ac, H3K56ac, H2AK9ac, H3K18ac, H4K5ac, H2AK5ac, H3K9me1, H3K4ac, H2BK5ac, H3K14ac, H3K79me1, H3K23ac, H2BK15ac, H3K4me2, H2BK12ac, H4K91ac, H4K20me1, H2BK20ac, H4K8ac, “or a combination thereof.

Embodiment 21. The modulator of embodiment 19, an enhancer-binding transcriptional activator (e.g., bromodomain-containing protein 4 (BRD4)) binds to the epigenetic modification.

Embodiment 22. The modulator of embodiment 19, wherein the epitranscriptomic modification comprises N6-methyladenine (m6A), N1-methyladenosine (m1A), inosine (I), pseudouridine (′), 5-methylcytosine (m5C), methylguanosine (m1G), N6,N6-dimethyladenosine (m62A), 4-thiouridine (s4U), or a combination thereof.

Embodiment 23. The modulator of any one of the preceding embodiments, the transposable element belongs to ERV1, ERV2, or MaLR families.

Embodiment 24. The modulator of any one of the preceding embodiments, the transposable element belongs to LTR16, LTR16A, LTR16A1, LTR16A2, LTR16B, LTR16B1, LTR16B2, LTR16C, LTR16D, LTR16D1, LTR16D2, LTR16E1, LTR16E2, ERV3-16A3_LTR, MER39, THE1A, THE1B, THE1C, THE1D, MSTA, MSTA1, MSTB, MSTB1, MSTB2, MSTC, MSTD, MLT1A0, MLT1A1, MLT1C, MLT1D, MLT1E, MLT1E1, MLT1E2, MLT1F, MLT1F1, MLT1F2, MLT1G1, MLT1G3, MLT1H, MLT1H1, MLT1I, MLT1J, MLT1J1, MLT1J2, MLT1K, MLT1L, MLT1M, MLT1N2, MLT1O, MLT2A1, MLT2A2, MLT2B1, MLT2B2, MLT2B3, MLT2B4, MLT2B5, MLT2C1, MLT2C2, MLT2D, MLT2E, MLT2F, MLT1A, MLT1B, MLT1E1A, MLT1E3, MLT1G, MLT1H2, MLT1C2, MLT1F-int, MST-int, MLT-int, MLT1H-int, MLT1J-int, THE1-int, HERV16, ERVL-E, HERVL, ERVL-B4, ERV3-16A3_I, LTR33, LTR50, LTR78, LTR41, LTR41B, HERV3-int, LTR13, HERVL18-int, MER66C, MER66D, or LTR19C subfamilies.

Embodiment 25. The modulator of any one of the preceding embodiments, wherein the transposable element comprises a sequence that is at least 60%, at least 70%, at least 80%, at least 90%, or at least 95% identical to at least a fragment of a sequence selected from of SEQ ID NOs: 4, 10, 14, 15, 18, 27-32, and 34-38.

Embodiment 26. The modulator of embodiment 25, wherein the transposable element comprises a sequence at least 60%, at least 70%, at least 80%, at least 90%, or at least 95% identical to at least a fragment of SEQ ID NO: 4.

Embodiment 27. The modulator of embodiment 26, wherein the transposable element comprises a sequence of SEQ ID NO: 5, SEQ ID NO: 6, or wherein the transposable element or a fragment thereof is located in chr3: 45,818,736-45,818,762 (hg38) or chr3: 45,818,689-45,818,712 (hg38).

Embodiment 28. The modulator of any one of the preceding embodiments, wherein the modulator activates or inhibits the RNA expression level of the transposable element.

Embodiment 29. The modulator of embodiment 28, wherein the modulator comprises a nucleic-acid guided endonuclease complex, wherein the nucleic acid targets the transposable element.

Embodiment 30. The modulator of embodiment 29, wherein the modulator is a CRISPR-directed DNA editing complex.

Embodiment 31. The modulator of embodiment 29, wherein the modulator is a CRISPR-directed RNA editing complex.

Embodiment 32. The modulator of embodiment 29, wherein the modulator is an ASO-directed RNA editing complex.

Embodiment 33. The modulator of embodiment 28, wherein the modulator comprises a nucleic acid molecule that hybridizes to the transposable element.

Embodiment 34. The modulator of embodiment 33, wherein the nucleic acid molecule is a small interfering RNA (siRNA), a microRNA (miRNA), an inhibitory double stranded RNA (dsRNA), a small or short hairpin RNA (shRNA), an antisense oligonucleotide (ASO), a piwi-interacting RNA (piRNA), a heterogeneous nuclear RNA (hnRNA), a small nuclear RNA (snRNA), or an enzymatically-prepared siRNA (esiRNA) or the precursors thereof.

Embodiment 35. The modulator of embodiment 34, wherein the nucleic acid molecule is an ASO.

Embodiment 36. The modulator of embodiment 35, wherein the ASO is a Gapmer or a mixmer.

Embodiment 37. The modulator of embodiment 36, wherein the ASO is about 6-50 nucleotides long.

Embodiment 38. The modulator of embodiment 37, wherein the ASO is about 12-30 nucleotides long.

Embodiment 39. The modulator of any one of embodiments 35-38, wherein the ASO comprises at least 9 consecutive nucleotides with no more than 3 mismatches from SEQ ID NOs: 1-3.

Embodiment 40. The modulator of any one of embodiments 35-38, wherein the nucleic acid molecule comprises a nucleic acid sequence at least 80%, at least 85%, at least 90%, at least 95%, identical to a sequence selected from SEQ ID NOs: 1-3.

Embodiment 41. The modulator of any one of embodiments 35-40, wherein the ASO is a Gapmer comprising a central region of consecutive DNA nucleotides flanked by a 5′-wing region and 3′-wing region, wherein at least one of 5′-wing region and 3′-wing region comprises a nucleic acid analogue, wherein the nucleic acid analogue comprises one or more ribose modifications, one or more backbone modifications, one or more nucleobase modifications, or a combination thereof.

Embodiment 42. The modulator of embodiment 41, wherein the nucleic acid analogue comprises an LNA.

Embodiment 43. The modulator of embodiment 42, wherein the LNA comprises a beta-D-oxy LNA, an alpha-L-oxy-LNA, a beta-D-amino-LNA, an alpha-L-amino-LNA, a beta-D-thio-LNA, an alpha-L-thio-LNA, a 5′-methyl-LNA, a beta-D-ENA, or an alpha-L-ENA.

Embodiment 44. The modulator of embodiment 43, wherein the LNA comprises a beta-D-oxy LNA.

Embodiment 45. The modulator of any one of embodiments 41-44, wherein the 5′-wing region comprises at least two LNAs.

Embodiment 46. The modulator of any one of embodiments 41-44, wherein the 5′-wing region comprises three consecutive LNAs.

Embodiment 47. The modulator of any one of embodiments 41-46, wherein the 3′-wing region comprises at least one LNA.

Embodiment 48. The modulator of any one of embodiments 41-46, wherein the 3′-wing region comprises two consecutive LNAs.

Embodiment 49. The modulator of any one of embodiments 41-48, wherein one or more phosphodiester backbone is a phosphorothioate backbone.

Embodiment 50. The modulator of any one of embodiments 41-48, wherein each phosphodiester backbone is a phosphorothioate backbone.

Embodiment 51. A modulator comprising an antisense oligonucleotide (ASO), wherein the ASO comprises at least 9 consecutive nucleotides with no more than 3 mismatches from 5′-CGGAGGCATGAGGTAG-3′ (SEQ ID NO: 1).

Embodiment 52. The modulator of embodiment 51, wherein the ASO comprises a nucleic acid sequence of 80%, at least 85%, at least 90%, at least 95% identical to SEQ ID NO: 1.

Embodiment 53. A modulator comprising an antisense oligonucleotide (ASO), wherein the ASO comprises at least 9 consecutive nucleotides with no more than 3 mismatches from 5′-TGAGCAGGTTAGCACT-3′ (SEQ ID NO: 2).

Embodiment 54. The modulator of embodiment 53, wherein the ASO comprises a nucleic acid sequence of 80%, at least 85%, at least 90%, at least 95% identical to SEQ ID NO: 2.

Embodiment 55. A modulator comprising an antisense oligonucleotide (ASO), wherein the ASO comprises at least 9 consecutive nucleotides with no more than 3 mismatches from 5′-CTACCTCATGCCTCCG-3′ (SEQ ID NO: 3).

Embodiment 56. The modulator of embodiment 55, wherein the ASO comprises a nucleic acid sequence of 80%, at least 85%, at least 90%, at least 95% identical to SEQ ID NO: 3.

Embodiment 57. A synthetic polynucleic acid comprising a nucleic acid sequence of a transposable element or a fragment thereof, wherein the synthetic polynucleic acid induces or facilitates a change of a state of a cell from a first cellular state to a second cellular state.

Embodiment 58. The synthetic polynucleic acid of embodiment 57, wherein the first cellular state or the second cellular state comprises an extracellular-cellular-matrix synthesizing state, a cytokine-secreting state, an inflammatory state, a cell dividing state, a cell pausing state.

Embodiment 59. The synthetic polynucleic acid of embodiment 58, wherein the first cellular state is the extracellular-cellular-matrix synthesizing state.

Embodiment 60. The synthetic polynucleic acid of embodiment 58 or 59, wherein the second cellular state is the inflammatory state.

Embodiment 61. The synthetic polynucleic acid of any one of embodiments 57 to 60, wherein a transposable element transcript that is transcribed from the transposable element modulates a functional RNA.

Embodiment 62. The synthetic polynucleic acid of embodiment 61, wherein the functional RNA comprises a messenger RNA (mRNA), a long noncoding RNA (lncRNA), a circular RNA, a small nuclear RNAs (snRNAs), a small nucleolar RNAs (snoRNAs), a microRNA, an X-inactive specific transcript (xist) RNA, or a combination thereof.

Embodiment 63. The synthetic polynucleic acid of embodiment 62, wherein the functional RNA comprises the lncRNA.

Embodiment 64. The synthetic polynucleic acid of any one of embodiments 57 to 63, wherein the transposable element transcript and the lncRNA are transcribed from the same strand.

Embodiment 65. The synthetic polynucleic acid of any one of embodiments 57 to 63, wherein the transposable element transcript and the lncRNA are transcribed from a different strand.

Embodiment 66. The synthetic polynucleic acid of any one of embodiments 57 to 65, wherein the transposable element or the transposable element transcript is associated with pulmonary fibrosis.

Embodiment 67. The synthetic polynucleic acid of embodiment 66, wherein the transposable element transcript is altered in a cell affected by pulmonary fibrosis compared to a healthy cell.

Embodiment 68. The synthetic polynucleic acid of embodiment 67, wherein the transposable element transcript is increased in a cell affected by pulmonary fibrosis compared to a healthy cell.

Embodiment 69. The synthetic polynucleic acid of any one of embodiments 57 to 68, wherein the transposable element is bound by or bears a binding motif being bound by a pulmonary-fibrosis-related transcription factor.

Embodiment 70. The synthetic polynucleic acid of embodiment 69, wherein the pulmonary-fibrosis-related transcription factor comprises friend leukemia integration 1 transcription factor (FLI1), interferon regulatory factor 3 (IRF3), interferon regulatory factor 4 (IRF4), (ETS like-1 protein (ELK1), activator protein 1 (AP-1), C-terminal binding protein 2 (CTBP2), Spi-1 Proto-Oncogene (SPI1/PU.1), transcription factor 7 (TCF7), transcription factor 7-like 1 (TCF7L1), transcription factor 7-like 2 (TCF7L2), transcription Factor 21 (TCF21), CCAAT enhancer binding protein beta (CEBPB), forkhead box protein A1 (FOXA1), forkhead box protein A2 (FOXA2), forkhead box protein 04 (FOX04), forkhead box protein L1 (FOXL1), forkhead box protein L2 (FOXL2), forkhead Box M1 (FOXM1), T-box transcription factor 4 (TBX4), T-box transcription factor 5 (TBX5), odd-skipped related 1 (OSR1), early B-cell factor 1 (EBF1), early B-cell factor 2 (EBF2), early B-cell factor 3 (EBF3), twist-related protein 1 (TWIST1), SMAD family member 2 (SMAD2), SMAD family member 3 (SMAD3), nuclear factor-1 (NF1), NK6 homeobox 1 (NKX6-1), NK2 homeobox 1 (NKX2.1), RUNX family transcription factor 2 (RUNX2), POU class 2 homeobox associating factor 1 (POU2AF1), high mobility group AT-hook 2 (HMGA2), lymphoid enhancer-binding factor 1 (LEF1), paired related homeobox 1 (PRRX1), tumor protein p63 (TP63), forkhead box protein P1 (FOXP1), Meis homeobox 1 (MEIS1), TGFB induced factor homeobox 1 (TGIF1), paired related homeobox 2 (PRRX2), TEA domain family member 1 (TEAD1), TEA domain family member 2 (TEAD2), TEA domain family member 3 (TEAD3), TEA domain family member 4 (TEAD4), signal transducer and activator of transcription 1 (STAT1), signal transducer and activator of transcription 3 (STAT3), signal transducer and activator of transcription 6 (STAT6), nuclear factor-kappa B1 (NFKB1), nuclear factor-kappa B2 (NFKB2), AT-rich interactive domain-containing protein 3A (ARID3A), forkhead box protein S1 (FOXS1), forkhead box protein J2 (FOXJ2), forkhead box protein I1 (FOXI1), forkhead box protein F1 (FOXF1), forkhead box protein E1 (FOXE1), forkhead box protein A3 (FOXA3), forkhead box protein D3 (FOXD3), MYC-Associated Zinc Finger Protein (MAZ), Zinc Finger Protein X-Linked (ZFX), Zinc Finger Protein Y-Linked (ZFY), transcription factor 12 (TCF12), transcription factor AP-2 gamma (TFAP2C), CCCTC-Binding Factor (CTCF), CCCTC-Binding Factor Like (CTCFL or BORIS), Myogenin (MYOG), Nuclear transcription factor Y subunit alpha (NFYA), Nuclear transcription factor Y subunit beta (NFYB), Nuclear transcription factor Y subunit gamma (NFYC), Upstream stimulatory factor 1 (USF1), Upstream stimulatory factor 2 (USF2), Jun Proto-Oncogene/AP-1 Transcription Factor Subunit (JUN), JunD Proto-Oncogene/AP-1 Transcription Factor Subunit (JUND), Fos Proto-Oncogene/AP-1 Transcription Factor Subunit (FOS), FOS Like 1/AP-1 Transcription Factor Subunit (FOSL1), MYC Associated Factor X (MAX), MAF BZIP Transcription Factor F (MAFF), or a combination thereof.

Embodiment 71. The synthetic polynucleic acid of any one of the preceding embodiments, wherein the transposable element transcript is bound by or targeted by an RNA-binding protein.

Embodiment 72. The synthetic polynucleic acid of embodiment 71, wherein the RNA-binding protein comprises of DDX3, RBM3, HuR, or a combination thereof.

Embodiment 73. The synthetic polynucleic acid of any one of embodiments 57 to 72, wherein the transposable element comprises an epigenetic modification or the transposable element transcript comprises an epitranscriptomic modification.

Embodiment 74. The synthetic polynucleic acid of embodiment 73, wherein the epigenetic modification comprises H3K27Ac, H3K4me3, H3K4me1, H4K16ac, H3K27me3, H3K79me2, H3K36me3, H2AFZ, H3K9ac, H3K4me2, H4K20me1, H2BK120ac, H3K56ac, H2AK9ac, H3K18ac, H4K5ac, H2AK5ac, H3K9me1, H3K4ac, H2BK5ac, H3K14ac, H3K79me1, H3K23ac, H2BK15ac, H3K4me2, H2BK12ac, H4K91ac, H4K20me1, H2BK20ac, H4K8ac, ‘or a combination thereof.

Embodiment 75. The synthetic polynucleic acid of embodiment 74, an enhancer-binding transcriptional activator (e.g., bromodomain-containing protein 4 (BRD4)) binds to the epigenetic modification.

Embodiment 76. The synthetic polynucleic acid of embodiment 73, wherein the epitranscriptomic modification comprises N6-methyladenine (m6A), N1-methyladenosine (m1A), inosine (I), pseudouridine (Ψ), 5-methylcytosine (m5C), methylguanosine (m1G), N6,N6-dimethyladenosine (m62A), 4-thiouridine (s4U), or a combination thereof.

Embodiment 77. The synthetic polynucleic acid of any one of embodiments 57 to 76, wherein the transposable element belongs to ERV1, ERV2, or MaLR families.

Embodiment 78. The synthetic polynucleic acid of any one of embodiments 57 to 76, the transposable element belongs to LTR16, LTR16A, LTR16A1, LTR16A2, LTR16B, LTR16B1, LTR16B2, LTR16C, LTR16D, LTR16D1, LTR16D2, LTR16E1, LTR16E2, ERV3-16A3_LTR, MER39, THE1A, THE1B, THE1C, THE1D, MSTA, MSTA1, MSTB, MSTB1, MSTB2, MSTC, MSTD, MLT1A0, MLT1A1, MLT1C, MLT1D, MLT1E, MLT1E1, MLT1E2, MLT1F, MLT1F1, MLT1F2, MLT1G1, MLT1G3, MLT1H, MLT1H1, MLT1I, MLT1J, MLT1J1, MLT1J2, MLT1K, MLT1L, MLT1M, MLT1N2, MLT1O, MLT2A1, MLT2A2, MLT2B1, MLT2B2, MLT2B3, MLT2B4, MLT2B5, MLT2C1, MLT2C2, MLT2D, MLT2E, MLT2F, MLT1A, MLT1B, MLT1E1A, MLT1E3, MLT1G, MLT1H2, MLT1C2, MLT1F-int, MST-int, MLT-int, MLT1H-int, MLT1J-int, THE1-int, HERV16, ERVL-E, HERVL, ERVL-B4, ERV3-16A3_I, LTR33, LTR50, LTR78, LTR41, LTR41B, HERV3-int, LTR13, HERVL18-int, MER66C, MER66D, or LTR19C subfamilies.

Embodiment 79. The synthetic polynucleic acid of embodiments 57 to 78, wherein the transposable element comprises a sequence that is at least 60%, at least 70%, at least 80%, at least 90%, or at least 95% identical to at least a fragment of a sequence selected from of SEQ ID NOs: 4, 10, 14, 15, 18, 27-32, and 34-38.

Embodiment 80. The synthetic polynucleic acid of embodiment 78, wherein the transposable element comprises a sequence at least 60%, at least 70%, at least 80%, at least 90%, or at least 95% identical to at least a fragment of SEQ ID NO: 4.

Embodiment 81. The synthetic polynucleic acid of embodiment 80, wherein the transposable element comprises a sequence of SEQ ID NO: 5, SEQ ID NO: 6, or the transposable element or a fragment thereof is located in chr3: 45,818,736-45,818,762 (hg38) or chr3: 45,818,689-45,818,712 (hg38).

Embodiment 82. A pharmaceutical composition comprising the modulator of any one of embodiments 1-56 or the synthetic polynucleic acid of any of embodiments 57-81, and a pharmaceutically acceptable salt, excipient, or derivative thereof.

Embodiment 83. A kit comprising the modulator of any one of embodiments 1-56 or the synthetic polynucleic acid of any of embodiments 57-81, or the pharmaceutical composition of embodiment 82.

Embodiment 84. A method of preventing, alleviating, or treating pulmonary fibrosis in a subject in need thereof, the method comprising administering to the subject an effective amount of the modulator of any one of embodiments 1-56 or the synthetic polynucleic acid of any of embodiments 57-81, or the pharmaceutical composition of embodiment 82.

Embodiment 85. The method of embodiment 84, wherein the modulator of any one of embodiments 1-56 or the synthetic polynucleic acid of any of embodiments 57-81, or the pharmaceutical composition of embodiment 82 reduces expression of a fibrosis-related marker in a fibroblast affected by the pulmonary fibrosis.

Embodiment 86. The method of embodiment 85, wherein the fibrosis-related marker comprises smooth muscle α actin (ACTA2), alpha 1 chain of collagen type I (COL1A1), alpha 1 chain of collagen type 3 (COL3A1), fibroblast activation protein (FAP), fibronectin 1 (FN1), periostin (POSTN), collagen triple helix repeat containing 1 (CTHRC1), alpha 1 chain of collagen type 10 (COL10A1), matrix metalloproteinase 11 (MMP11), thrombospondin 2 (THBS2), latent transforming growth factor beta binding protein 2 (LTBP2), Integrin-α11 (ITGA11), or a combination thereof.

Embodiment 87. The method of any one of embodiments 84-86, wherein the modulator of any one of embodiments 1-56 or the synthetic polynucleic acid of any of embodiments 57-81, or the pharmaceutical composition of embodiment 82 restores an expression of a set of genes that are aberrantly expressed in idiopathic pulmonary fibrosis (IPF) comparable to a healthy cell.

Embodiment 88. The method of embodiment 87, wherein the set of genes comprises one or more genes identified in Table 3.

Embodiment 89. The method of any one of embodiments 84-88, wherein the modulator of any one of embodiments 1-56 or the synthetic polynucleic acid of any of embodiments 57-81, or the pharmaceutical composition of embodiment 82 releases a cell from the subject from quiescence.

Embodiment 90. The method of any one of embodiments 84-89, wherein a percentage of fibrotic lesion surface is decreased, wherein the percentage of fibrotic lesion surface is measured by picrosirius red (PSR) staining.

Embodiment 91. The method of any one of embodiments 84-90, wherein a parameter reflecting lung functions of the subject is improved.

Embodiment 92. The method of embodiment 91, wherein the parameter comprises enhanced pause (Penh) measured by whole-body plethysmography, tidal volume, a functional lung capacity, lung resistance, lung compliance, functional residual capacity (FRC), vital capacity (VC), inspiratory capacity (IC), forced vital capacity (FVC), forced expiratory volume over 100 ms (FEV100), total lung capacity (TLC), duration of expiration (Te), time the subject was occluded (Tocclude), chord compliance between 0-10 cm H₂O [ml/cmH2O] (Cchord), compliance at 50% vital capacity [ml/cmH2O] (Cfvc50), residual volume (RV), or a combinational thereof.

Embodiment 93. The method of any one of embodiments 84-92, the modulator of any one of embodiments 1-56 or the synthetic polynucleic acid of any of embodiments 57-81, or the pharmaceutical composition of embodiment 82 reduces altered immune response in pulmonary fibrosis.

Embodiment 94. The method of embodiment 93, wherein more M0 macrophages or fewer profibrotic M2 macrophages are observed in the pulmonary tissue.

Embodiment 95. The method of any one of embodiments 93-94, wherein fewer white blood cells or fewer macrophages are observed in a bronchioalveolar lavage of the subject.

Embodiment 96. The method of any one of embodiments 93-95, wherein a number of total T cells or activated T cells is decreased, or a number of immunosuppressive Ctla4-expressing cells is increased in the pulmonary tissue.

Embodiment 97. The method of any one of embodiments 93-96, wherein a cell-cell communication between a fibroblast and an immune cell (e.g., a T cell, an M0, M1, or M2 macrophage) is decreased in the pulmonary tissue.

Embodiment 98. The method of any one of embodiments 84-97, wherein the modulator of any one of embodiments 1-56 or the synthetic polynucleic acid of any of embodiments 57-81 is encapsulated in a liposome or coupled with a nanoparticle.

Embodiment 99. The method of any one of embodiments 84-98, wherein the modulator of any one of embodiments 1-56 or the synthetic polynucleic acid of any of embodiments 57-81 is encoded by a transgene in an expression vector.

Embodiment 100. The method of any one of embodiments 84-99, wherein the administering is performed intratracheally, oropharyngeally, orally, nasally, intravenously, intraperitoneally, or intramuscularly.

Embodiment 101. The method of embodiment 100, wherein the administering is a targeted delivery to a lung tissue of the subject.

Embodiment 102. The method of embodiment 101, wherein the administering is in a form of aerosol or via a microsprayer.

Embodiment 103. A method of reprogramming a myofibroblast into a new cell type, the method comprising contacting the myofibroblast with an effective amount of the modulator of any one of embodiments 1-56, the synthetic polynucleic acid of any of embodiments 57-81, or the pharmaceutical composition of embodiment 82.

Embodiment 104. The method of embodiment 103, wherein the new cell type is selected from an early fibroblast precursor cell, a cell that is Top2a, Cenpa or ki67 positive, a proliferating fibroblast, or a combination thereof.

Embodiment 105. The method of embodiment 104, wherein the early fibroblast precursor cell is a mesothelial cell, an early fibroblast cell, a mid fibroblast cell, or a mesenchymal cell.

Embodiment 106. The method of embodiment 104, wherein the cell that is Top2a, Cenpa or ki67 positive is a mesenchymal progenitor cell.

Embodiment 107. The method of embodiment 106, wherein the mesenchymal progenitor cell is an epithelial progenitor cell, an endothelial progenitor cell, or a fibroblast progenitor cell.

Embodiment 108. The method of any one of embodiments 103-107, wherein an activity of early B cell factor 1 (Ebf1) is decreased.

Embodiment 109. A method of slowing or reversing aging of a pulmonary tissue in a subject, the method comprising administering to the subject an effective amount of the modulator of any one of embodiments 1-56 or the synthetic polynucleic acid of any of embodiments 57-81, or the pharmaceutical composition of embodiment 82.

Embodiment 110. The method of embodiment 109, wherein an expression of a gene specific to a young population that is selected from a supplementary table 7 of Chow et al in Example 5 is increased in the pulmonary tissue.

Embodiment 111. The method of any one of embodiments 109-110, wherein an expression of a gene specific to an old population that is selected from a supplementary table 7 of Chow et al in Example 5 is decreased in the pulmonary tissue.

Embodiment 112. The method of any one of embodiments 109-111, wherein an expression of a gene specific to a proliferating fibroblast as referred to in Example 6 is increased in the pulmonary tissue.

Embodiment 113. The method of any one of embodiments 109-112, wherein an upregulated proteostasis is observed in the pulmonary tissue.

Embodiment 114. The method of embodiment 113, wherein an expression of Zmpste24 or Lamp2 is increased in the pulmonary tissue.

Embodiment 115. The method of any one of embodiments 109-114, wherein an upregulated macro-autophagy is observed in the pulmonary tissue.

Embodiment 116. The method of embodiment 115, wherein the expression of Atg5 or Atg7 is increased in the pulmonary tissue.

Embodiment 117. The method of any one of embodiments 109-116, wherein a more active telomerase is observed in the pulmonary tissue.

Embodiment 118. The method of embodiment 117, wherein an expression of Tert or Terc is increased in the pulmonary tissue.

Embodiment 119. The method of any one of embodiments 109-118, wherein an age-related inflammation or an age-related tissue repair capability is improved in the pulmonary tissue.

Embodiment 120. The method of embodiment 119, wherein more M0 macrophages or fewer profibrotic M2 macrophages are observed in the pulmonary tissue.

Embodiment 121. The method of any one of embodiments 119-120, wherein fewer white blood cells or fewer macrophages are observed in a bronchioalveolar lavage of the subject.

Embodiment 122. The method of any one of embodiments 119-121, wherein a number of total T cells or activated T cells is decreased, or a number of immunosuppressive Ctla4-expressing cells is increased in the pulmonary tissue.

Embodiment 123. The method of any one of embodiments 119-122, wherein a cell-cell communication between a fibroblast and an immune cell (e.g., a T cell, an M0, M1, or M2 macrophage) is decreased in the pulmonary tissue.

Embodiment 124. The method of any one of embodiments 109-123, wherein an activity of sry-related HMG box 2 (Sox2) is increased.

Embodiment 125. The method of any one of embodiments 109-124, wherein an age-related epigenetic alteration (e.g., an altered chromatin plasticity and an altered histone modification) is reversed.

Embodiment 126. The method of any one of embodiments 109-125, wherein cellular senescence measured by Imna nuclear lamina gene, an age-associated alteration in PI3K-AKT pathway, or an age-associated alteration in Ras-MEK-ERK pathways is reversed.

Embodiment 127. A method for diagnosing or monitoring pulmonary fibrosis in a subject, the method comprising:
a) obtaining a biological sample derived from the subject;
b) detecting an amount and/or an activity of a plurality of biomarkers from the biological sample, wherein the plurality of biomarkers comprise a transposable element transcript transcribed from a transposable element, wherein the transposable element comprises a sequence that is at least 60%, at least 70%, at least 80%, at least 90%, or at least 95% identical to at least a portion of a sequence selected from of SEQ ID NOs: 4, 10, 14, 15, 18, 27-32, and 34-38; and
c) diagnosing the subject with pulmonary fibrosis or to have a high/higher chance to contract pulmonary fibrosis if the amount and/or the activity of the plurality of biomarkers is at least 50%, at least 60%, at least 70%, at least 80%, at least 90% higher when compared to a control.

Embodiment 128. A method of predicting severity and progression of pulmonary fibrosis in a subject, the method comprising:
a) obtaining a biological sample derived from the subject;
b) detecting an amount and/or an activity of a plurality of biomarkers from the biological sample, wherein the plurality of biomarkers comprise a transposable element transcript transcribed from a transposable element, wherein the transposable element comprises a sequence that is at least 60%, at least 70%, at least 80%, at least 90%, or at least 95% identical to at least a portion of a sequence selected from of SEQ ID NOs: 4, 10, 14, 15, 18, 27-32, and 34-38; and
c) predicting the subject to have a more severe or a progression of pulmonary fibrosis if the amount and/or the activity of the plurality of biomarkers is at least 50%, at least 60%, at least 70%, at least 80%, at least 90% higher when compared to a control.

Embodiment 129. A method for monitoring an efficacy of a pulmonary therapy in a subject, the method comprising:
a) obtaining a biological sample derived from the subject before and after the pulmonary therapy;
b) detecting an amount and/or an activity of a plurality of biomarkers from the biological sample, wherein the plurality of biomarkers comprise a transposable element transcript transcribed from a transposable element, wherein the transposable element comprises a sequence that is at least 60%, at least 70%, at least 80%, at least 90%, or at least 95% identical to at least a portion of a sequence selected from of SEQ ID NOs: 4, 10, 14, 15, 18, 27-32, and 34-38; and
c) concluding the pulmonary therapy is effective if the amount and/or the activity of the plurality of biomarkers after the pulmonary therapy is at least 50%, at least 60%, at least 70%, at least 80%, at least 90% lower when compared to the amount and/or the activity of the plurality of biomarkers before the pulmonary therapy.

Embodiment 130. A method for evaluating or monitoring pulmonary tissue regeneration in a subject, the method comprising:
a) obtaining a biological sample derived from the subject at an earlier time point and a later time point;
b) detecting an amount and/or an activity of a plurality of biomarkers from the biological sample, wherein the plurality of biomarkers comprise a transposable element transcript transcribed from a transposable element, wherein the transposable element comprises a sequence that is at least 60%, at least 70%, at least 80%, at least 90%, or at least 95% identical to at least a portion of a sequence selected from of SEQ ID NOs: 4, 10, 14, 15, 18, 27-32, and 34-38; and
c) concluding with a positive pulmonary tissue regeneration if the amount and/or the activity of the plurality of biomarkers at the later time point is at least 50%, at least 60%, at least 70%, at least 80%, at least 90% lower when compared to the amount and/or the activity of the plurality of biomarkers at the earlier time point.

Embodiment 131. A method for evaluating or monitoring efficacy of a treatment to induce pulmonary reprogramming in a subject, the method comprising:
a) obtaining a biological sample derived from the subject before and after the treatment;
b) detecting an amount and/or an activity of a plurality of biomarkers from the biological sample, wherein the plurality of biomarkers comprise a transposable element transcript transcribed from a transposable element, wherein the transposable element comprises a sequence that is at least 60%, at least 70%, at least 80%, at least 90%, or at least 95% identical to at least a portion of a sequence selected from of SEQ ID NOs: 4, 10, 14, 15, 18, 27-32, and 34-38; and
c) concluding the treatment is effective if the amount and/or the activity of the plurality of biomarkers after the treatment is at least 50%, at least 60%, at least 70%, at least 80%, at least 90% lower when compared to the amount and/or the activity of the plurality of biomarkers before the treatment.

Embodiment 132. The method of embodiment 127 or 128, wherein the pulmonary fibrosis is idiopathic pulmonary fibrosis (IPF).

Embodiment 133. The method of any one of embodiments 127-132, wherein the sample is an alveolar type 2 cell (AT2 cell) or a lung fibroblast.

Embodiment 134. The method of any one of embodiments 127-133, wherein the method further comprises d) administering to the subject the modulator of any one of embodiments 1-56, the synthetic polynucleic acid of any of embodiments 57-81, or the pharmaceutical composition of embodiment 82.

Embodiment 135. The method of embodiment 134, wherein the modulator, the synthetic polynucleic acid, or the pharmaceutical composition is administered intratracheally, oropharyngeally, orally, nasally, intravenously, intraperitoneally, or intramuscularly.

Embodiment 136. The method of any one of embodiments 127-135, wherein the plurality of biomarkers comprises a fibrosis-related marker.

Embodiment 137. The method of embodiment 136, wherein the fibrosis-related marker comprises smooth muscle α actin (ACTA2), alpha 1 chain of collagen type I (COL1A1), alpha 1 chain of collagen type 3 (COL3A1), fibroblast activation protein (FAP), fibronectin 1 (FN1), periostin (POSTN), collagen triple helix repeat containing 1 (CTHRC1), alpha 1 chain of collagen type 10 (COL10A1), matrix metalloproteinase 11 (MMP11), thrombospondin 2 (THBS2), latent transforming growth factor beta binding protein 2 (LTBP2), Integrin-α11 (ITGA11), or a combination thereof.

Embodiment 138. The method of any one of embodiments 127-137, wherein the plurality of biomarkers comprise one or more genes identified in Table 3 and Table 5.

Embodiment 139. A microarray comprising a plurality of probes that hybridize to a transposable element transcript transcribed from a transposable element, wherein the transposable element comprises a sequence that is at least 60%, at least 70%, at least 80%, at least 90%, or at least 95% identical to at least a fragment of a sequence selected from of SEQ ID NOs: 4, 10, 14, 15, 18, 27-32, and 34-38.

1.-51. (canceled)

52. A method of treating a subject suspected to have pulmonary fibrosis, the method comprising:
a. selecting the subject based on an amount and/or an activity of a plurality of biomarkers from a biological sample derived from the subject,
wherein the plurality of biomarkers comprises a transposable element transcript described from a transposable element, wherein the transposable element comprises a sequence that is at least 60% identical to at least a portion of a sequence selected from any one of SEQ ID NOs: 4, 7-11, and 14-121; and
b. administering to the subject a modulator, wherein the amount and/or the activity of the plurality of biomarkers is at least 50% higher when compared to a control,
thereby treating the subject.

53. The method of claim 52, wherein the transposable element comprises a sequence that is at least 60%, at least 70%, at least 80%, at least 90%, or at least 95% identical to at least a portion of a sequence selected from any one of SEQ ID NOs: 4, 10, 14, 15, 18, 27-32, and 34-38.

54. The method of claim 52, wherein the amount and/or the activity of the plurality of biomarkers is at least 60%, at least 70%, at least 80%, at least 90% higher when compared to a control.

55. The method of claim 52, wherein the pulmonary fibrosis is idiopathic pulmonary fibrosis (IPF).

56. The method of claim 52, wherein the biological sample is a lung biopsy sample.

57. The method of claim 52, wherein the modulator comprises a nucleic acid molecule that hybridizes to the transposable element transcript, and activates or inhibits RNA expression of the transposable element.

58. The method of claim 57, wherein the nucleic acid molecule is an antisense oligonucleotide (ASO).

59. The method of claim 58, wherein the ASO is a mixmer or a Gapmer, wherein the Gapmer comprises a central region of consecutive DNA nucleotides flanked by a 5′-wing region and 3′-wing region, and wherein at least one of 5′-wing region and 3′-wing region comprises a nucleic acid analogue.

60. The method of claim 58, wherein the ASO is about 12-30 nucleotides long.

61. The method of claim 59, wherein the ASO comprises a nucleic acid sequence complementary to a portion of a sequence having a sequence homology of at least 55%, at least 60%, at least 65%, at least 70%, at least 75%, at least 80%, at least 85%, at least 90% of any one of SEQ ID NOs: 4, 7-11, and 14-121.

62. The method of claim 59, wherein the ASO comprises a nucleic acid sequence complementary to a portion of a sequence having a sequence homology of at least 55%, at least 60%, at least 65%, at least 70%, at least 75%, at least 80%, at least 85%, at least 90% of any one of SEQ ID NOs: 4, 10, 14, 15, 18, 27-32, and 34-38.

63. The method of claim 59, wherein the nucleic acid analogue comprises an LNA or 2′-methoxyethyl(2′-MOE).

64. The method of claim 58, wherein the ASO comprises one or more backbone modifications.

65. The method of claim 64, wherein the one or more backbone modifications comprise a phosphorothioate backbone.

66. The method of claim 52, wherein the modulator is administered intratracheally, oropharyngeally, orally, nasally, intravenously, intraperitoneally, intramuscularly, or by inhalation.

67. The method of claim 52, wherein the plurality of biomarkers further comprises a fibrosis-related marker.

68. The method of claim 67, wherein the fibrosis-related marker comprises smooth muscle α actin (ACTA2), alpha 1 chain of collagen type I (COL1A1), alpha 1 chain of collagen type 3 (COL3A1), fibroblast activation protein (FAP), fibronectin 1 (FN1), periostin (POSTN), collagen triple helix repeat containing 1 (CTHRC1), alpha 1 chain of collagen type 10 (COL10A1), matrix metalloproteinase 11 (MMP11), thrombospondin 2 (THBS2), latent transforming growth factor beta binding protein 2 (LTBP2), Integrin-α11 (ITGA11), or a combination thereof.

69. A method for diagnosing or monitoring pulmonary fibrosis in a subject, the method comprising:
a. obtaining a biological sample derived from the subject;
b. detecting an amount and/or an activity of a plurality of biomarkers from the biological sample, wherein the plurality of biomarkers comprises a transposable element transcript transcribed from a transposable element, wherein the transposable element comprises a sequence that is at least 60%, at least 70%, at least 80%, at least 90%, or at least 95% identical to at least a portion of a sequence selected from any one of SEQ ID NOs: 4, 7-11, and 14-121; and
c. diagnosing the subject with pulmonary fibrosis or to have a high/higher chance to contract pulmonary fibrosis if the amount and/or the activity of the plurality of biomarkers is at least 50%, at least 60%, at least 70%, at least 80%, at least 90% higher when compared to a control.

70. The method of claim 69, wherein the biological sample is a lung biopsy sample.

71. The method of claim 69, wherein the transposable element comprises a sequence that is at least 60%, at least 70%, at least 80%, at least 90%, or at least 95% identical to at least a portion of a sequence selected from any one of SEQ ID NOs: 4, 10, 14, 15, 18, 27-32, and 34-38.


SEQUENCE LISTING (121 sequences)

SEQ ID NO: 1 - DNA, 16 bp, synthetic construct
  source 1..16: mol_type = other DNA
cggaggcatgaggtag

SEQ ID NO: 2 - DNA, 16 bp, synthetic construct
  source 1..16: mol_type = other DNA
tgagcaggttagcact

SEQ ID NO: 3 - DNA, 16 bp, synthetic construct
  source 1..16: mol_type = other DNA
ctacctcatgcctccg

SEQ ID NO: 4 - DNA, 489 bp, Homo sapiens
  source 1..489: mol_type = genomic DNA
tgtagcagacgctgtcggtgccccgcccatatcccctcggcccttaccatttcagtgcacgccggcccgacttccaactgccagcacctgcatctctttgcctgagggctttctctggccgccggagcccgctctgcccacgcgcanggcaggccggaagtgccggggaattaacgccccccgggagcagccctcaaccaatgactgacgggagttggtgnataaataccccagctccctcgcccctcgggtgggataactctgaggcgcgtgttctacactgtctcccagagttccccagcgggattgagctccagttgcccacagtggtaacttgctcgataacgcaccctttattggctnccttcccttccctgtctcacttccccactcccctaccggtgtttcctgggatcacctcccaaataaactacttgcactcgaatccttgtctcagggtctgcttctgggggaacccaaactaagaca

SEQ ID NO: 5 - DNA, 182 bp, Homo sapiens
  source 1..182: mol_type = genomic DNA
tcagtgggattgagcgtggtggtccacagtgctaacctgctcattaacacacattttatctcctttcttccctttcctacctcatgcctccgtaccctcaccccactttctgggatcccctccccacttcaaccaagatccctgcctcagggttttgcttttgggggagctgaaataaaaca

SEQ ID NO: 6 - DNA, 334 bp, Homo sapiens
  source 1..334: mol_type = genomic DNA
ctgcctgtctttcctgaagacgttctctctctggccctgttcccagggcaggctgaaggacaaaggacaggaaggtaaggtcccaggagtgacccccagtgtggaggatgagagttgttggataaatgtcccagctccctcctccctccatggggcagttctggggcatgttctgctcagacctcaatgcagtcccagtgggatgaaccgaagttccctctggcagggatcctctctctagggtacctgcactcctgccttcactgtgcttcctgagatcacccccaaaatcaactgcttgcccccaaatctttgtcatagggttttcaagaga

SEQ ID NO: 7 - DNA, 538 bp, Homo sapiens
  source 1..538: mol_type = genomic DNA
tgtagcagacgcctctggtgccccgcgtcacatcccctcggcccacctctgatttcagccgcagctgcggtggacagttccgtgcgagctcagactcacctttgctgacagcgtcccacctcaagcgcgcgccgtgcgtctttctgctttctgccccagggccttctccgacgccgcgggagcccgctcggcccgcgcgcaagcgcagcccggaagtgcgggggagttaacgcccccgggggcaaccctcaaccaatgggggacgggagccggtggataaatgccccagcctcccgtccttcaggtggacaattctgggaggcattctgtacgcttctcaggaggtcccagcgggatcgagccccngttgcccacagcggcgacctcgataacgcacccttatattggcttttcctccttccctgtctcacttccccgctccctcactcctgcttcctgggatcacctcccaaataaactacctgcacccaagtccttgtctcaggctctgctttcgggggaacccaaactaagacag

SEQ ID NO: 8 - DNA, 364 bp, Homo sapiens
  source 1..364: mol_type = genomic DNA
tgatatggtttggctgtgtccccacccaaatctcatcttgaattgtagctcccataattcccacgtgtcgtgggagggacccggtgggaggtaattgaatcatgggggcgggtctttcccgtgctgttctcgtgatagtgaataagtctcacgagatctgatggttttataaaggggagttcccctgcacangctctcttgcctgccgccatgtaagacgtgnctttgctcctccttcgccttccgccatgattgtgaggcctccccagccacgtggaactgtgagtccattaaacctctttcctttataaattacccagtctcgggtatgtctttatcagcagcgtgaaaacggactaataca

SEQ ID NO: 9 - DNA, 1580 bp, Homo sapiens
  source 1..1580: mol_type = genomic DNA
gtaaattggtaccagtagagtggggcgctgctgaaaagatacccgaaaatgtggaagcgactttggaactgggtaacaggcagaggttggaacagtttggagggctcagaagaagacaggaaaatgtgggaaagtttggaacttcctagagacttgttgaatggctttgaccaaaatgctgatagtgatatggacaataaagtccaggctgaggtggtctcagatggagatgaggaacttgttgggaactggagcaaaggtgactcttgttatgttttagcaaagagactggcggcattttgcccctgccctagagatttgtggaactttgaacttgagagagatgatttagggtatctggcggaagaaatttctaagcagcaaagcattcaagaggtgacttgggtgctgttaaaggcattcagttttanaagggaagcagagcataaaagttcggaaaatttgcagcctgacgatgcgatagaaaagaaaancccattttctgaggagaaattcaagccggctgcagaaatttgcataagtaacgaggagccgaatgttaatccccaagacaatggggaaaatgtctccagggcatgtcagaggccttcgcggcagcccctcccatcacaggcccggaggcctaggaggaaaaaatggtttcgtgggccgggcccagggtccccgtgctgtgtgcagcctagggacttggtgccctgcgtcccagccgctccagccgtggctgaaaggggccaacgtacagctcgggccgtggcttcagagggtgcaagccccaagccttggcagcttccacgtggtgttgagcctgcgggtgcacagaagtcaagaattgaggtttgggaacctccgcctagatttcagaggatgtatggaaacgcctggatgcccaggcagaagtttgctgcaggggcggggccctcatggagaacctctgctagggcagtgcggaagggaaatgtggggtcggagcccccacacagagtccctactggggcaccgcctagtggagctgtgagaagagggccaccgtcctccagaccccagaatggtagatccaccgacagcttgcaccgtgcgcctggaaaagccgcagacactcaacgccagcccgtgaaagcagccgggagggaggctgtaccctgcaaagccacaggggcggagctgcccaagaccatgggaacccacctcttgcatcagcgtgacctggatgtgagacatggagtcaaaggagatcattttggagctttaagatttgactgccccgctggatttcggacttgcatggggcctgtagcccctttgttttggccaatttctcccatttggaatggctgtatttacccaatgcctgtacccccattgtatctaggaagtaactaacttgcttttgattttacaggctcataggcggaagggacttgccttgtctcagatgagactttggactgtggacttttgagttaatgctgaaatgagttaagactttgggggactgttgggaaggcatgattggttttgaaatgtgaggacatgagatttgggaggggccaggggcggaa

SEQ ID NO: 10 - DNA, 438 bp, Homo sapiens
  source 1..438: mol_type = genomic DNA
tgtagtggatgctgtggtgcgccgcccagatcgnnccccccttcaggaccgaggcactcattcccccagctgccgggagtgttggctgctgacggctcncagctgagtccctctccgggaattgccctcggccgaagggagctgcctcgcccaaggttacgcccccttcccgggggcagcccgcatccaatgactggtcgatgcgggggtacaaaggcctcagtcctcaattcgggacaactctgaagggccatcccagctccagagctccccgtgggatcggctgaggcctctgttgcgactgcatcgcagttcaacttctccctctgcccaatcctgcttccttcactccctcacaggtgttgntcccgagagcactccccaataaacctcctgcacgcaaatctccgtctcagagtctgtttcccggggaaccca

SEQ ID NO: 11 - DNA, 5226 bp, Homo sapiens
  source 1..5226: mol_type = genomic DNA
catgagtggtaccgggagtggtccgnagaaaacaggcggtaagatggggttttgggactggctcactcaccgcccggcggacagaggataccatcctggcacaaggtggcggcccagttgctaaagatttcaccggtggtgacctgggaaaatgtgtcccagnnnttggaaggggaatgcccttgcaggtgcaatgattcaggcatttgaaagatatggggaganaatgcctacaaggacagcggagttggctggttattgctaagttgtattgatgccctgcagagggataatgagaaactgagggcngttaacaagcagttaaaggctaagtgtgagagccagagggcctctttgntggtagcttacaaagaggcccttatctcctgcagtggaagagcagacanagctgaggagcagactcaggatctaatagttagagtcgcagagctccagagacgtttgaatgctcagccaaggcgaggtctgttatgctaaagntaaggcccttggttgggaaaacctgggacggaacacgggatggggacatctgggtggatgcccccgaagatnttggctctccagactcctctgaaccctcngagcctgcagaagtggcccacccctccctantaagagctagcactcctcttgcgcccgcgctggaagacgntgcagaggcctctccccgcaaggcaacaggtgccccctctcagganctgcccccacctcccctcctggccgccaggccgataactagggttaagtcncagcataacccggctggggacgtgctgggcctgataagggaggaaagggactataccccaaaggagctgcaagaattagccagcatgtaccggcaggagccaggggagtatacncntgggactggattttgagggtgcttgatcaagggggccggaatataagcggtaagactggataanggaagaatttattgacttgggagcactttctcgggatacaggatttaacaccctggcaaggaccccaggagatggtgcaaactcgctgctaggatggctcctagaagcgagaaaaatggcccacgctgagcgaagtngaaatgccngaattgccntggcagacggtagaggaagggattaaaggctcagggaagtgggcatgctggaatggatatnattatgtaaggccggaagacccaccagangattatgttccacgggagggcccacaccattcaccaaggccatnaggaatgcgctggtgagaggggcaccagcanntcactaagaagttcagtggtggctctcctctgcaggccagggctgacggtaggagaggccgttacagaactgggctcgctgatagcaatggggatgataggaccccgaaacaatagaggccaggtggcggcgcttaaccgccagaagccaggnggncgcaattatcgtaatgaccggaaggtcggagtggcagccaagggggcctgacccaagagagttgtggagatggttaatagaacacggcgtccctaggggcaaaatagatngacaagggtgctgcttaacttgtaacagcagaaaaaatctgggaggctgagggcggtcgccccaataaaaaagtcacgatcccttgcccagtttccggacctgagccagttttcagacccggaacccattgactgaagaggnggccgggtccccaggaggaaggacactgcaatgattcttctcnttcctcccgnagggacctatggccatttactcgggtaactgtacactggggaaagggaatacccagacatttcgaggactgttggacacagggtctgagttgacattgatacccggagacccgaagcgtcatcatggccccctgttagagtgggggcatatgggggccaggtaataaatggagtcctggccaaggtccggctcacagtgggtccactgggtccangaacccagtggtcatttccccggtccccgaatgtataattgggtagttggagtaacccccacattggntccttggcctgtggggtaagagctatcatagtggggaaggccaagtggaagcctctgaaactgcccnccnccnccattcctagccaagatagtaaattattatatcctctcgaggaagtggcggatgcagattagtgccaccnttaaagacctaaaggatgcaggggtggtggtccccatcatatctccatttaattcaccagtctggcccctgcagaaaccggatggatcctggagaatgacngtagactaccgcaaactcaaccaagtagtagccccgattgcagctgctgtgccagatgtggtatctttgctagagcagattaacacggcctcaggtacatggtatgcggccattgatttggcgaatgcgttcttttccattccnatcagaaaagaggatcagaaacattcacatgaacaggaaanaatatacatttacagttttgccccagggctatgttatcttctcctctgtcataataagtccgaagagatctggaccgtctggacatcccgcagaacatcacattgatccattacatcgatgacatcatgctgatcggacnggatgagcaagaggtggctacgttaggccttggtaagacacatgcgctgagggtggagataaccctacgaagattcagcgnnnggacctgccacntcagtaaagtttttaggggtccagtggtcnggggcatgccgggacatcccctccaaagtaaaagacaaattgttgcatcttgcatctcctaccacgaagaaggaagcacaacgcctggtaggcctctttgggttctggaggcagcatattccacacctgggaatactgctccggcccatntaccgggtgacacggaaggctgccagctttgagtgggcccagagcaggaaagggctctgcagcaggtccaggctgcggtgcaagcagccctgccgcttgggccatacgacccggcagaccctatggtgttggaggtgtcagtggtgggaaaagatgcngtgtggagtttatggcaagccccagtgggagaatcacaacgcaggcccctggggttctggagcaaggctgcaagtcttttgaaaaacagctcttnnnatgctactggtagaganggaacgcttaccatgggacaccaagtgacacgnancaaaatgtaccccgtcatgagctgggttctgtcggacccaccaagtcataaggtcgggcgggcccagcagcaatccatcgtaagatggaagtggtacatccgggatcgagcacgaggaggaccacaagtaagctgcatgagcaggtagcccagacccccatgtcacccaccacngttgcaccagcgctccttcagctcacacctatggccgtatggagnagaggtgtgntcctgatcgaccagctgaaggaggaggaaaaangcccgagcttggttcacggatgggtcggctcggtatgtgggtgcaagccgaaaatggacggcggcttgcactacagccncactcaggggtggccttgaaagacagtggngagggaaaatcttcccaatgggcagagcttcgggcggtgcacctggtcatccactttgtgtggaaggagaagtggcccgaggtnagaatatatacggactcatgggcagtggcgaatggcttggccggctggtcaggggcctggaaggagaaagattggaagatcggagacaaggaggtctggggantagaggcatgtggatggacntatgggagtgggcacgaagtgtgaagatctttgtatcacatgttaacgcccaccagagagcatccaccacggaagaggcactaaacaaccaagtagacaaaatgactcggccagttgacgtcagccagcctctgtcatcggccaccccangtgctggcacaatgggcacatgaacggagtggccacggtggcagagatggaggctacgcatgggcccaacagcatggactcccactcaccaaggctgatctagctacttgctgccncnnctgaatgtccaacctgccagcaacagagaccgatccccgatatggcaccattcctcgaggagaccaaccagccacttggtggcaagttgactacattgggccccttccatcctggaagggncagcggttcatcctnacaggaatacatattccgggtatgggtttgcctttcctgcccgcagggcctcagccagcaccactatccgagggcttacggagtgcctgatccacnggcatgggatcccacacaacatngcatcngaccagggacccactttacagcaaaggaggtgcgggagtgggcccatgaccatgggatccactggtcntatcacataccgcaccatccagaagctgccggcctgatagagcgntggaacggcctnctgaaggcacagctgaagcgccagctcggagacgatactctgcgaggatggggcgccatccttcaggatgcagtgtccccaataggaagaatacatgggtccgggaaccaaggggtggaagcaggagtgtngccccacttaccatcactcccagtgacccactgggggantttgtgcttcccgtccccgcaactctgggctctgcaggnttagaggtcctggtccccaaagggggnacgctctcgccaggggacacagcgtcccattgaactntanagctacggctgccgcctgggcactttgggctccttgtgtccagggaccagcaggcaagaagaggagtcaccatcttggcaggggtaattgaccctgatcatcaggaggaggtagggctgctnttacacaatgggggcagggaggaatacgtntggaacccaggtgatccacttgggcgcctcttggtactcccttgcccaattntgactgtaaatggacaagtgcagcaaccccggcctgagaagggcatggtnaccaggggctcagacccctcaggaatgagggtctgggtcacgccaccaggtaagccaccgagaccagcagagtgntagctgagggtgaggggaatctagaatggatagtggaggagggagacgatgagtatcagttgcggccccgagaccaactgagcgacgggggctgtagttcgtcccactaacctccctcttctaagtttccctcaggaagagaggcccacnggaatcctggaggagctgctcctgaaccgaacgtgtatggagaagtggatccgngcggcgcaaggggtggac

SEQ ID NO: 12 - DNA, 20 bp, synthetic construct
  source 1..20: mol_type = other DNA
ccacagtgctaacctgctca

SEQ ID NO: 13 - DNA, 20 bp, synthetic construct
  source 1..20: mol_type = other DNA
tcccccaaaagcaaaaccct

SEQ ID NO: 14 - DNA, 5226 bp, Homo sapiens; identical to SEQ ID NO: 11
  source 1..5226: mol_type = genomic DNA

SEQ ID NO: 15 - DNA, 422 bp, Homo sapiens
  source 1..422: mol_type = genomic DNA
tgtggcagccacggaggtgcgccgctcggatctcccttcaagaaagaacttgccgttcagctgcgaggagtgcggttagctgacagcctccagctgttagcgccttcaggatccgcctcagctttcgagccgaggccacgctcttcccgggcagcccccagccaatgactgagcanggcgggggtacnagggcctggccatttctgcccaacgcgggactcctctaacgggcaatctttgctccggaagagctccccgttgggttggccgagactttgtcagatctgcatcgcggtctgaggctctccctgcccaatcctgcttcctctccttttntctttcacaggcgttaccccccaataaacctcttgcactcctaactccgtctcagcgtctgcttcccggaggacccaactgacaca

SEQ ID NO: 16 - DNA, 5714 bp, Homo sapiens
  source 1..5714: mol_type = genomic DNA
gattttggtaccgagagtggttctagaggaacagaattttaaggatgagttttctgaattggttctggggtttctggaattggctctctaatctgattagatttaaagacgctaatgactctatttccagtagtaaagagagcactgatagtccatggcgtgatctggcaatagagatacgcaaaatatcnccattggatactcctaatcaaccacttataagaagcaagganctgggtgactntgtatatgatactttcgaacatttttggnaaactaacgaatataatgagattggctggttgctcctaatgtcgctggacaaagtggngaaagaaaaggatgagctcagggattcgaattcccagctcaagcgccgcataaatgacctgaaagcttctatgtgtgccctgaaggagacccttatctcctgtagccgcagggctgagattgctgaaaatcaaacgcagaatctcatcctgcgactggctgaattacaacgcaagttgaactcccagcctcgcagggtgtctactgttaaagtgagggcattgattgggaaagaatgggatcctgnaagttggaatggggacgtgtgggaagaccctgatgaagctggggacattgagcccctaaattctgatgagtcttctttgccagtggaagnggcctccccacccccagtggaagcggcctccccacccccagtggtagcggcctctccacccccgtctgaggggattaaccctgcattgcctgaggaaactgtaatggcctcccctgaggcagttgccatgcaagacaatgctgattctcctcaggacccacccccaccacccctctttgcttctagacctataactagactcaagtcccagcaggcccctaaaggtgaggtacaaagtgtgacccatgaggaggtgcgctacactccaaaagaactacttgagttttctaatttatacagacagaaatccggggaacatgtgtgggaatggatattaagggtgtgggataatggtggaaggaacataaagttggatcaggccgaatttattgatatgggcccactaagcagagattctgcatttaatgttgcagctcggggagttagaaagggctctaacagtttgtttggttggttggctgaaacatggaccaaaaggtggcccacagtgagcgaattggaaatgccggacctgccttggtttaatgtagaggaagggattcaaaggcttagggagattggaatgttagagtggatttgtcatttaagacctactcacccacactgggagggtccagaagacatacctttcaccannactgtgagaaataaatttgtgaggggagccccagcatccttgaagagctctgtgatcgctcttctctgtaggccagaccttacagtgggaactgcagccactgaattgggaaacctaaatgcaatgggagtaattggatcccggggtggcaggggccaagtggcggcactcaaccgccaaaggcaaggtgggcgtggttaccgtaatggacagcagagtcaaagcagcaatcagaatagtctgactcgcgcagacctatggcgttggctagttgatcatggtgttcctagaagtgaaatagataggaagcctactaaattcttacttgatctgtataagcagaaaagttctaggtcaagtgaacaaaagtctaacttgaatcataaaaacagagagtcacggcccctcaatcaattcccagacttgagccagtttacagacccagaaccccttgaatgaaggggaggccgggtccccttgaggaaggaccccggtacactgccaaaaatttatactgttaatctttctcccagccttccccaaagggacctacggccttttaccagggtaactgtgcattggggaaaaggaaataatcagacctttcggggactactggacactggctctgaactgacactaattccaggagacccaaaacgtcactgtggtccaccagtcagagtaggggcttatggaggtcaggtgatcaatggagttttagctcaggtccgtctcacagtgggcccagtgggtccccgaacccatcctgtggttatttccccagttccggaatgcataattggaatagacatactcagcagctggcagaatccccacattggttccctgacctgtggagtgagggctattatggtgggaaaggccaagtggaagccactagaactgcctctacctaggaaaatagtaaaccaaaagcaataccgcattcctggagggattgcagagattagtgccaccatcaaggacttgaaagatgcaggggtggtgattcccaccacatccccattcaactcgcctatttggcctgtgcagaagacagatggatcttggagaatgacagtggattatcgtaagcttaaccaggtggtgactccaattgcagctgctgtaccagatgtggtttcattgcttgagcaaattaacacatcccctggtacctggtatgcagctattgatctggcaaatgcctttttctccatacctgtcaataaggnccaccagaagcagtttgctttcagctggcaaggccagcaatacaccttcactgtcctacctcaggggtatatcaactctccagccctatgtcataatttagttcgcagggatcttgatcgcctttcccttccacaagatatcacactggtccattacattgatgacattatgctgattggacctagtgagcaagaagtagcaactactctagacttattggtaagacatttgcgtgtcagagggtgggaaataaatccgacaaaaattcaggggccttctacctcagtgaaatttctaggggtccagtggtgtggggcatgtcgagatatcccttctaaggtgaaggataagttgttgcatctggcccctcctacaaccaaaaaagaggcacaatgcctagtgggcctctttggattttggaggcaacatattcctcatttgggtgtgttactccggcccatttaccgagtgacccgaaaagctgctagttttgagtggggcccagaacaagagaaggctctgcaacaggtccaggctgctgtgcaagctgctctgccacttgggccatatgatccagcagatccaatggtgcttgaagtgtcagtggcagatagggatgctgtttggagcctttggcaggcccctataggtgaatcgcagcgcaggcccttaggattttggagcaaagccctgccatcctctgcagataactactctccttttgagaaacagctcttggcctgctactgggccttagtagagactgaacgcttaaccatgggccaccaagttaccatgcgacctgagctgcccatcatgaactgggtgttatctgacccaccaagccataaagttgggcgtgcacagcagcactccatcatcaaatggaagtggtatatacgtgatcgggcccgagcaggccctgaaggcacaagtaagttacatgaagaagtggcccaaatgcccatggtccccactcctgctacactgccttctctctcccagcctgcacctatggcctcatggggagttccctacgatcagttgacagaggaagagaagactcgggcctggtttacagatggttctgcacgatatgcaggcaccacccgaaagtggacagctgcagcactacagcccctttctgggacatccctgaaggacagtggtgaagggaaatcctcccagtgggcagaacttcgagcagtgcacctggttgttcactttgcttggaaggagaaatggccagacgtgcgattatataccgattcatgggctgtggccaatggtttggctggatggtcagggacttggaaggaacatgattggaaaattggtgacaaggaaatttggggaagaggtatgtggatagacctctctgaatgggcaaaaaacgtgaagatatttgtgtcccatgtgaatgctcaccaaagggtgacctcagcagaggaggattttaataatcaagtggataggatgacccgttctgtggataccagtcagcctctttccccagccacccctgtcatcgcccaatgggctcatgaacaaagtggccatggtggcagggatggaggttatgcatgggctcagcaacatggacttccactcaccaaggccgacctggctacggccaccgctgagtgcccaatctgccagcagcagagaccaacactgagtccccgatatggcaccattccccggggtgatcagccagctacctggtggcaggttgattacattggaccgcttccatcatggaaggggcagcgttttgttcttactggaatagacacttactctggatacggatttgccttccctgcacgcaatgcttctgccaaaactaccatccgtggacttacagaatgccttatccaccgtcatggtattccacacagcattgcttctgatcaaggaactcacttcacagcaaangaagtgcggcaatgggcccatgctcatggaattcactggtcttaccatgttccccaccatcctgaagcagctggcttgatagaacggtggaatggccttttgaagactcagttacagcgccagctaggtggcaataccttgcagggctggggcaaggttctccagaaggctgtatatgctctgaatcagcgtccaatatatggtgctgtttctcccatagccaggattcacgggtccaggaatcaaggggtggaaatgggagtggcaccactcactattacccctagtgacccactagcaaaatttttgcttcctgttcccgcgaccttatgctctgctggcctagaggtcttagttccaaagggaggaatgcttccaccaggagacacaacaatgattccattgaactggaagttaagactgccacccggccactttgggctcctcatgcctctgaatcaacaggcaaagaagggagttactgtgctggctggggtgattgatcctgactaccaaggggaaattggactgctactccacaatggaggtaaggaagagtatgtctggaatacaggagatcccttagggcgtctcttagtattaccatgccctgtgattaaggtcaatggaaaactacaacaacccaatccaggcaggactactaatggcccagacccttcaggaatgaaggtttgggtcaccccaccaggtaaagaaccacgaccagctgaggtgcttgctgaaggcaaagggaatacggaatgggtagtggaagaaggtagttataaataccagctacgaccacgtgaccagttacagaaacgaggactgtaattgtcatgagtatttcctccttattttgttatgaatatgtttgtgtgtatatatacatatattaagcaaatatctttgttttctttcctctcttattcccttatcatgtaacataagatgtattgactttatatcatagtatttaagtattgttaattttacatcatagtatttaagttacgggatatcaaggagaagagtaaacatcactcaaggactttacctcctcttctggggaaggggttagtgcgttttcggttgtacgcaggatagttgtatcatgttaggcggaattatgaccttgttattgtctttatttggagattaagtatggtttaaggagatgcgtatgggtgccaagttgacaaggggtggact

SEQ ID NO: 17 - DNA, 5667 bp, Homo sapiens
  source 1..5667: mol_type = genomic DNA
attattggtaccgggagtggttccaggggaacagaaccttaaggatgggaatctggaattggttctctgatctgattagatttaaaggcgctaatgaccctgtttccagtggtaaaggggacactggtagtccatggcatgcagtggcaaaaagttactcaaattatcacctgtggncacctgtaatcaagtgcctatagaaggcaaggctttgggtgaccangtanttgctgccntagaacattttagtggaaataaggagtataatgangttggttggttgcttctaantgcgctggagaacttggagaaagaaaatgatgagctcagggctttaaattcccagctcaagntccgggtaagggaccngaaagcttctatgactgccctgaaagaaacccttatctcctgtagccgcagggctgagatttctgaaaaccaaacccaaagtctnatcctgcgggtggctgaattacaacgcaaattgaattcncaacctcgcagggtntcttntgttaaagttagggcattgattgggaaggaatgggaccctgaaaattggaatggggacatntgggcggattccgatgaagctggggaccttgaacccctaaattctgccgagccttctttgccagtagaagcagccctttcncccctgtctgaggaggttagtctccccttgcctgaagaanctgtaatggcctcccctgaggtagttgccttgcaaggnantgctgattctcctcaggacctacccccaccacctctcnttgcttctagacctataactagactcaagtcccagcaggccccnaggggtnaggtacaaagtgtgacccatgaggaggtannntacacaccaaaagaattgcaagattttnccaatttatatcgacagaaacctggggaatatgtgtgggaatggatnctaagggtgttggatcanggtggaaggaatataacgttggatcgggccgaatttattgatatgggtncactaagcagagattctggattcaatgtgttagctcgagnagctggaagtggctctaacagtttgcttggttggttgactgaaacntggactcaaaggtggcctacantnaatgaagttgagatgccagaacttccttggtatantgtagaggaaggnatccaaaggcttagggagatnggaatgttggagtggatttatcatgtaagacctgctcacctacaccctctaactatgtccccgggagggtccagggacactcccttcaccaaggcnttgagaaatacattngtgaggggagcaccagcatccttgaagagctctgtggtggctnttctctgtaggccaggnatgacggtgggagatgctgccattgaaatgggctccctganttcaatggggatgatgggatcccggggtggcagaggccaagtggcggcacttaaccgccagagacaaggtgggcgcggttaccgtaatgggcagcagagccaaagcggtaatcagaatggtttgacccgcagagatctttggcgntggctaattgatcatggtgtccctaggantgaaatagatgggcagcctactaaagtcttacttgatttgtataagcagaaaagctctaggtctggtgaacagaagtctgacttgagtcaccaacggagagtcacggcccctcanccagttcccagacttgagccagttcacagccagaccccttgaatgaaggggaggccgggtccccttgaggaaggaccctgcnacactgccaaaaatntatactgtaaatcttcctccnagccttccccaaagggacctgcggccatttaccagggtgactgtgcactggggaaagggaaatanccagacttttnagggattactggacactggctctgaactgacgctaattcctggagacccaaaacgccactgtggtccaccagtcagagtaggggcttatggaggtcaggtgatnaatggagttttggctcgagtccgtctcacagtgggcccagtgggtccncgaacccaccctgtggttatttccccagttccngaatgcatagttggaatagacatactcagcaactggcagaatccccacattggttccctgacccgtggagtgagggctattatggtaggaaaggccaagtggaagccnctggaactgcctctncctaccaaaatagtaaaccaaaagcaataccgcatccctggaggaattgcagagattagtgccaccatcaaagacttgaaagatgcaggggtggtgattcctaccacatccccatttaactcgcctgtttggcctgtgcagaagacagatggatcttggagaatgacagtggattatcgtaaacttaatcaggtggtgactccaattgcagctgctgttccagatgtggtntctttactggagcaaatcaacacancccctggcacctggtatgcagctattgatctggcaaatgcttttttctctatacctgttagtaaagaccaccagaagcagtttgctttcacctggcagggncagcagtacaccttcactgtcttgcctcagggctatgtcaactctccngctctctgtcataatntagtccgcagggaccttgatcgtcttnncattccacaggacatcacgctggtccactacattgatgacatcatgctgattggacctggtgagcaggaagtagcaagtactctagacgccttggtaagacacatgcgtgccagagggtgggagataaatcccacgaaaattcaggggcctgccacctcggtgaagtttctaggggtccagtggtctggggcatgtcgagatatcccttccaaggtgaaggacaagttgctgcatctngcncctcctaccactaagaaagaggcacaatgcttggtgggcctctttggattttggaggcaacatataccncatttgggcgtgctgctccgacccatttaccgagtaacccgnaaggctgccagttttgagtggggcccagagcaagagaaggctctgcagcaggtccaggctgcngtgcaagctgctctgccacttgggccntatgacccagcagatccaatggtgctcgaagtgtctgtggcagatagggatgctgtatggagcctctggcaagccccnataggngaatcacagcgcagacccctaggattttggagcaaagccatgccatcttctgcagataactattctccttttgagaaacagctcctggcttgctactgggccctggtagagactgaacgcctgaccatgggncaccaagtnaccatgcgacctgagctgcccatcatgaactgggtgttatctgacccaccaagccataaagttgggcgtgcacagcagcantccatcatcaagtggaagtggtatatacgagatcgggctcgagcaggtccngaaggcacaagtaagttgcatgagcaggtggctcagactcccatggcncctactcctgctgcattgcctcctctccctcaacccgcacctatggcctcatggggagttccctatgaccagttgactgaggaagaaaaaactcgggcctggtttacagatggttctgcacgatatgctggcaccanccgaaagtggacggctgcagcactacagccccactcaggggtggccctgaaggacagtggtgaagggaaatcctcccagtgggcagaacttcgagcagtgcacctggttgtccactttgcctggaaggagagatggccagaggtacggatctacactgattcatgggcagtggctaacggtttggctggatggtcagggacttggaaggaacangattggaagattggtgacaaggaggtctggggaagaggtatgtggatggacctctcggaatgggcacagagtgtgaagatatttgtgtcccatgtgaatgctcaccaaagggcanccncngcagaggaggntctcaataatcaggtggacaagatgacccgttctgtggatgtcagtcagcctctttccccagccaccccngtgcttgctcaatgggctcatgaacaaagtggccatggtggcagggatggaggctatgcatgggctcagcaacatggacttccnctcaccaaggctgatctggctacngccactgctgagtgcccaacctgccaacagcagagaccaacgctgagcccccgatatggcaccattccccggggggaccagccagccacctggtggcaggttgattacattggaccncttccatcatggaaggggcagcgatttgtcctcactggaatagacacttattctggatatggatttgccttccctgcccgcaatgcttctgccagnaccaccatccgtggacttacagaatgccttattcaccgtcatggtattccacacagcattgcttctgaccaaggaactcattttacagcaaangaagtgcggcaatgggctcatgcccatggaattcactggtcttaccacgtnccccatcacccngaagcagctggcctgatagaacggtggaatggcctnttgaagactcagttacggcgccagctgggngacaacaccttgnagggctggggtnntgtcctncaggatgcggtatatgctctgaatcagcgaccaatatatggtgctgtttctcccatagccagaatncacgggtccgggaatcaaggggtggaagtgggagtggctcctctcactattacncctaatgacccactngcaaaatttttgcttcccgtccccgcaactttgggctctgctggtttagaggtcttagttcccaagggaggaatgcttccaccaggggacacaacaatggttccattgaactggaagctgagactgccacctggccactttgggctcctcatgccactgaaccaacaggcaaagaagggagttactgtactggctggggtgattgatcctgattatcaaggggaaattgggttgctgctacacaatgggggcaaggaggantatgtctggaatncaggagatcctctggggcgcctcttagtactcccatgtccngtgataaaagttaatggaaaactacagcaacccaatanaggcaggaccgctaanggctcagacccttcaggaatgaaggtttgggtcaccccaccaggcaaagaaccacgaccagctgaggtgctngctgagggcaaagggaatatggaatgggtagtggaagaaggaagttataaataccagctacgaccncgtgaccagttgcagaaacgaggactgtagtagttatgngtatttcttccttgctttgatatgaatatatttgtgatatatatattaacnaatatctttnttttctttcctctctcattcccctactatctaacataagatgtgttaatagtagttaaccttatatctcagtatttaagttacaggatatcaaagggggantgtgactcagctagaagagnaatgaacatcacccagagatggataaagtgacntntgggactttgtatcctcttttggggagagggttagcgtgttttcggttgtacgagggatagttgcatcatgttaggcggaagcatgattttgctattgtctttatttggaagttaaatatggttnaaagaggtgtgtatggatgccgagttgacaaggggtggac

SEQ ID NO: 18 - DNA, 4996 bp, Homo sapiens
  source 1..4996: mol_type = genomic DNA
gttggtaccaggagtggtccgagaaagcagacgntnctaagatgggattttggagctggatcacccgccgnccggctggcaatgaggaccccatcactggtggtaggtggagcacggatagcccctggcacgaggtagcggtgcaattgttaaaactttcaccggtggtgaactgggatggnataccggtggaaagagaatgcactggcnggtgcaatgtntcaggcgtttgagaaatatgggangaattaantacatgnaaggacaatggaattggatggctgttgctaagcncgactgacgctctggaaaaagacaatgaaaggctgagagcgattaatcgncaattnaaagctaagtgtgaaagccagagggcctccttggcagcatataaagagactctcatctcctgcagccggagggcagagaaagctgaggatcaggcccaggacttaatcagagtagcagagctccagagaaggttgaattctcaaccnaggcaggtctgctatgccaaggtcagggccctggttgggaaagaatgggaccctganacntgggatggggacatctgggtcgatgcncctgaaaatcttgaatccccagattcccctgaaccctctgggcctgcagaagtggcccactcctccctgttaagggctagcactcnctccttgcttcgcgngaagacgatgcagaggcctctnccttngcaagacaacatgcgcccccctcaggatctgcccccacctcccctcctggccaccagaccaataactagggttaagtcacagcataacccggccggggaagtgctgggcctgntaagggaggaaagggactataccccaaaggagctgcaggacncgnctagccagcatgtaccggcaggaccgggagagtacgcatgggactggattctgagggtgctggatcaagggggncggaacataaagttggataagggagagtttatcgatntgggagcactctcccgngatacaggatttaacaccctggcaaggaccccgggagatggtgcnaacacgctgctaggatggctcctggaagcntggagaaagcgatggcccacactaagtgaagtagaaatgccagaactgccgtggcagacggtggaagaagggatcaaaaggctcagagaagtgggcatgctagagtggatatactatgtaaggccggaaaacccaccagatgactatgttccgcgggagggcccagaggacacnccatttaccaaagcnataaggaatgcgctggtgagaggggcaccagcatcactgagaagctcagtggtggctntcctctgcaggccagggctgacggtaggagangccgttacagaactgggctcnctgatagcaatggggatgataggaccccgaaataatagaggccaggtggcggcgcttaaccgtcagaagcaaggtgggcgcaattatcgtaatgnacggcaaggtcggagtggcagccgggggggcctgacccgcagagagctatggagatggttaatagaacacggcgtccctaggggcaagatagatgggcagccaacaggaagncatggacagttaatagaacatggcgtcctaggggcaagatagatgggcagccaacaagggtgtattgctcaactnaaacaancaaaagaaatcaaggatggatgancaggaggctgagggcagtcgccccaataaaaagtcacgatcccttgcccagtttccggacctgagccagttttcagacccggaacccattgactgaaggagaggccgggtccccaggaggaaggaccctgcaacaccacggcaagtgtacacggtaatgattcccccagtccttccccaaagggacctacggccatttactctgggtaaccgtacactggggaaaggggaatacccagacatttcgaggactgttggacacagggtccgagttgacattgatacccggagacccgaagcgtcatcatggccctcccgttagagtgggggcatatgggggccaggtaataaatggagtcctggcccaggtccggctcacagtgggtccactgggtccacggacccacagtggtcatttccccggtccccgaatgtataattggaatggacatacttggtagttggcanaacccccacattggttccttggcctgtggggtaagagctatcatagtggggaaggccaagtggaagcctctgaaactgccctcaccttctccggncaagatagtaaatcaaaaacaatatcgcatcctcggggtggagaatggcagagattagtgccacccttaaagacctaaaggatgcaggagntgtggtccccatcatatctccatttaattcaccagtncnggcccctgcaaaaaccagacggatcctggangacgacagtggactaccgcaaactcaaccaagtagtancccaattgcagctgctgtgccagatgtggtatctttgctagagcagattaacacggcctcaggtacgtggtatgcggccattgatctggcgaatgcgttcttttccatccctatcagaaaggaggatcagaagcagttcgcattcacntggaacggacaacagtatacatttacagtcttgccccagggctntgttaactctcctgccctctgtcataatatagtccgaagggacctggaccatctggacattccgcagaacatcacattggtccactatatcgatgacatcatgctaatcggaccggaatgagcaagaagtggcaagtacgttggaggccttggtaagacacatgcgctccagagggtgggagataaaccctacgaagattcaggggcctgccacatcagtgaagtttttaggggtccagtggtctggggcatgccgggacatccccccaaagtaaaggacaaattattgcatcttgcacctcccaccacnaagaaggaagcacaacgcctggtaggcctcttcgggttctggaggcagcatattccacacttgggaatactgctccgacccatntaccgggtgacgcgaaaggctgccagctttgagtggggcccaagcaggaaagggctctgtagctnaccgcatatcgtgtncgaancgcananncggncctccaagtcataaagtngggcgggcccagcagcagtccatcataaggtggaaatagnatatccgagattaagcccgagnaggaccagagggcgcaggtnagntncatgagcaggtagcccagacccccatgncacccaccacngttgcaccagcgcctctccttcggctcgcacctatggccatatngaggnntgtggtcccgtatgaccagctgaaggaaagctcgagcttggtttacaggatcagctcggtatgtgggcgcaagccaaaatangtggtggctgcactacagccccattcaggggtggccctgaaaggcagcagggagtgaaaatatcttcccagtgggcagagctgcgagcagtgcacctggtcatccactttncgtggaaggagaagtggcccgagatggtaatatatacggattnctggacagtggccaatggcctggtcggctggtcaggagcttggaaggaaaagattagaganagggaagtctggggtagaggcatgtggatagacatatnggaagtggcacgaaatacgaagatttttgtatcgntnntatgttaacgctcacangaaagcatcnaccatgaaagagncactgaacaaccaagtagacaaagtgacttcgaccactgacgttagccagccttcgtcccggcctcctcaganctggtncaacggacatctcaanaaagtagccatggtggcagagatggaggctacgcatgggcccaacagcatggactcctatncaccaagncgatctagctgctgccccgtntgaatgtccaacctgcagcaacagagaccaatgctctacctcnaatacggcactatttatggagaccacttagcggcgaattgactatattggacgccttccatcctggaanggncagngattcattctcacaggaatanatacntattccgggtatgggtttgcctttcctgcctgcanagcctcatcnagcatctgagggncttaggagtgcctgatccacaggcatggaatcccacacaacatagcatctgancagggcgctcacttcacagcaaagaagnggcacaagtgggtccanaataacgggatccacttatcntatcacgtaccgcaccatccagaagnagctgacctnatagaacgcttctgaaggcacagntgaagcaccagtngaggcgaactctgaaagaattgggtgccatcctccaggatgcagtatatacattgaatcanaggcctatncgtngggcnttgnntctaatngggagaatgggattaggaataagaggtgaaagcaggagtgtgcctactccccatcgtagtggcccacaaggatcacagcagtggtttgaacacggattggactactgatatccaggaatcgccgcacaagaaggngaatccacatttggcaggagtaattgaccntgatcancaggaggggtagggctgctntcgcanaatgaggaagaaaggaatatgtntggaaccaggtgatccncttgggtgcctcctggtactcccttgccccatttaactgtaaatggacaatnccaccgaaaagagctcggaccnctccggaatgaaggtttggntcacatcgccnggtaagccaccaagacctgtcgaagtgatagctgngtgaggganatctagaatggatagtagaagagggagacgatgagtaccagttncggccccgagaccaactgcagcaannggggctgtagttcgtcccactaacctccctcttctaagtttcgcttcagaaagagaagcttaaaggaataatggaggaactgctccnngaacctgngtggagaagtagatccgtcgagtacaaaggtggac

SEQ ID NO: 19 - DNA, 8426 bp, Homo sapiens
  source 1..8426: mol_type = genomic DNA
tttctggcgagccagccaggagtggagacgacaggtttgctgtctcctttgcctgtgggnctggagccccgggccgggggagacctgtgaccccaggcgccgcctgggagaacttcagcccggaggggagatcggctctcccgtgacccggcgcccctncccggcagcgcaacggaacctaaggggctacaggacgattccaggaacagcgcgctncaggaccgcggtaaggtttggggcccaaggcaggacccgtcccataaggacggaaggggagcctgatcacctcccggggtgtanctagtagtccgacccaggagctgggggcggcgagagtggctcgccaattcggatgaaacttacaccccaaccaagaanaggaactgggagtggggaagtgtgtgaatgcgtgtgaaagaggcggttccaggaggagccaatgcggggagtgacgtgtggggccgcaggtctcttagcgtggaccgtacgctccgagcgaagtgtggggccgaccgggactagtggcgaacgtcctccgnnnccgccgcatacggctnagggaggcgccccacaactcagtgattgtggtggtccgggttcggggntnatacgaaccctccantaaagctaagcggcgtctgaaaactcccgtaagggagacggtctaatcggtctgaagcgaaagtaaaagagtgagtgtgttgcgccgtaactgggaggaaatgggagggaagtcgtcaaaacccaccccattggaatgcatgttaaagaactttaagaaaggttntgcaggggattatggagtcaagttgaccccccagaggttgagaactctctgtgaattagaatggccctcttttggtgtcggatggccggccgaaggaactatagatagggaaacaattggccgtgtatttaaggtggtgacnggggtcggaggacagccagggcacccagaccaatttccttatattgactcatggctaaatatagtncagacncgaccagcatggntncagccctgcctagcggcttattgcaaaacgctcgtggctcgagccgagccnaaagtgaaagaaaaatcagcttcgccggcagccacggagntaaagggaaagccacaggaaggcaagagaagccagttttgcaggagccgccagaggaaacagaaattcctcctccntatgtcccagcctacccccctttaccgaggccaacggcccccgaggagccagattcaggngntaacacgccccaggtctcaccccgaagggaaggatcggagcctcgggaggccaaggagggaagtcaagatagtcaagcgggccgcctcngatctggccgcgcccgagctatgcaaatgcctctcagggagacgcgaggacccgtctattatgatgancagggccangtccaaggggggcaacggactttcatctaccagcccttttcaaccactgatctcctaaactggaaacaccatactccctcctacacggagaagccccaggccctcatagatctgatgcagtccatctttctgacacacaatccaacctggccagactgcaagcagctcctcctgacgctgtttaacaccgaggagcgccggagggtgacccaggcagccctccgctggctagaagccaatgcgccagaaggcgcanttaatgcccaggcatacgctcagggccagttcccagaagcagaccccnactgggacccagatgatgcaacccagcttcagcgtctgcagaggtaccgagaggcactcctgcaagggctaagaganggcggaaaaaaggcaatcaatataggaaagatctcagaggtgcttcagggagctgacgagagcccnagccagttttatgagagactctgtgaggcattccggctttacaccccgtttgaccctgaggctgctgaaaatcagcgcatggtgaatacggcatttgtagggcaagcccagggtgacatcaggcggaagctgcagaagctagaaggtttcgcaggcatgaatgccacccagcttatagaagtggccaccaaggtgtacgttaaccgtgaccaggaggcaaaaagggaagctgatcggaggcttaggaaaaaggccgatctgctggcggcagccctcacggaaagggaagctagcatcgcgaggggacgcggacgcggacgcggacgtggaaggggccaagctgggcagagacccgagagccggccgaggctaganagggatcaatgtgcgcggtgcaaaaagaagggacactggaaggatgagtgtccagagggcaatgaaggaaatggccaaggccgtgagacgagaaggccgccggccaagggctgccgcaccctgggggaaccagacaccgacctgatcgggctggcaggggctgaaggatatgaggactaggacagaccgggctccntctccttaggcccccaggagcccatggtcacattagaagtngggggccaactgatggactttatggtagacaccggggctgaacactcggtagtgacccggcccatagggccactatccaagaactatacaactattgtnggggctacnggggtcccagagaagaggccattctgccggccaaggaggtgtgtcataggaggacgagaagtccaacatgaattcctatacctcccaaattgcccagttcccttgctgggaagagacctactccaaaaactgcaagcacagattgcttttgggccgcaaggggatatgactttaaacctgactcacccaaaggccatggtgttaacccttaccgtcccgcaggctgaggaatggagactatacgcaaaanagncgccagaaccgggantaaatgaantgtntgggctacttagtaaaattcctggagtatgggctgaagataacccacccgggctggctgtaaatcaggcaccggtggtagtagagctaaaaccgggagcaactccggttcgggttcgccagtacccgcttccccgagaggccgtacggggcattcgcaaacacttagagcggctctncaaacacggaatcttagtccgatgccagtcaccctggaacactccactcttgccagtacggaagccagggtctggtgaatataggccggtgcaggacttgcgtgctgtaaaccaggccacggtgaccatccacccggtggtaccaaacccgtatactttaatgggactcattccggcaagtgccgcctggtttacnngcctagacttaaaggatgcnttcttctgtctccgcctggcaccaattagtcagcccatctttgcatttcaatgggacgattnagtcacaggcacagnggngcagctcacctggactagactcccacaagggttcaaaaactctcccacaatctttggagaagcactggcctcagacctcaaggcctacaccccgccaaatgacaactgcgccttgctncagtacgtagacgaccttcttctggcagccccaacccgagaggactgctaccaaggaacccaagacctcctccacctcctgtggaaggcaggntataaagtgtccaggaagaaggctcaaatctgccnngaaagngtcaagtatttaggcttcatagtaagccaaggggaacgncggcttggcagtgaacgaaagcaggctgtttgtgcacttccnactccaaccacccggcgtcaaataagagagttcttaggggcagcagggttctgccgcatctggatcccaaatttctcgctnatggctaagccattatatgaagccacaaagaggggggaaaaggagcccctcctctgggaggccgaccaggagaaggcntttaaacaaatcaaagaagccttaactcaggccccagccttaggactgccagatntaactaagcctttctttctgtatgtccacgagcgaaagggaatggctataggggtcctgactcaagtcataggatcatggcatcgcccggtggcgtacttatccaggcaattggactccgtggcgctnggatggcctccttgccttaaggcactagctgccaccgccctactggcacaagaagctaacaaactgactctaggacagcaactgaccatccgggtacgccgcactcggttataactttaatggaccagagagggcaccattggttatcaaatccgagaatgactcggtaccaggggctcctatgcgaaaatccccgcataactttagaaacngtgaacaccctnaacccggctaccttgctcccgatcgaaccgggagncccccttcatnactgtgtgganacggtagatgaggtgttctcaagccggagagatctnacagaccggcccctcggggacccggacgttgaatacttcacagatggaagcagtttcgtactagaaggggtccgccgagccgggtatgcagtggtgacnttggactcagtggtagaggcncagcctctgcctaccggaacntcngcccagaaggcagagctaatagccctgacaagagctcttttgctagcaaaagacaaaaaggtcaatatttacacngattccaaatatgcttttgccacnttgcatgttcatggggctatatacaaagaaagaggactcttaactgcggagggaaaaagaaataaagtacaaggaagaaattctacagctcttagatgctgtatgggccccaaagaaggtggctgtnatgcactgcagggggcaccaaaaggcaggaacactagaggccaaaggaaacagaaaggcagacagggaggcaaagcgggcagcaatgactactccgcattctaaagaggaagccctagctatgcctctcctcccggagnctcccctcccggagatcccaagttacnctccaaatgaaagggcctggtttgcccaagaanctggaaantacattgaaggaggatggtggaaattctccgatgggaggctagccatncctgaaatggtggcccccaaatttgtaaaacaattccaccaaggaactcacatgggaaaaacggcactagaaacgctactaggacgccatttctatgtgccacggctcactgccatcactcgagccgtttgcgaacaatgtctaacttgtgcccagaacaacccacgacaagggcccactcggccccccgggaattcaggaaataggagccacgccctgtgaaaacctgcttatggacttcaccgaactgccccgagcngggggctatcggtacatgctggtgctcgtttgcaccttttcaggatgggtcgaggctttccccaccaggacagagaaagcacgagaagtgactaaagtactgttaagagacattatccccagatttggactgcctctaactctagggtcagacaatggaccggcatttgtagctgaaatagttcagganctaacacggctgttaaaaataaaatggaagttacacacagcctaccggccgcagagctcaggaaaagtggagcgcatgaaccggacactcaagcagctactgaagaaatattgccaggaaactcatctgagatgggatcaggtcttgcccatggtcctcctccgagtcaggtgcacccccaccaaacaaactgggtattcgccctatgagattttgttcggccggccacccccaatcataggtcaaattaaaggtgatctccgngaactaggggaattaaccttaagaaggcaaatgcaggctttagggatagccatgcaaaangtccatggctgggtacgggaaagaatgcctataagcctgacagacccagcacacccctttaaacctggggactctgtttaggttaagaaatggaatccaaccactctaggacccatatgggatgggccccatactgtaatcttgtccactcccactgctgttaaagttgcaggnatcgtgccttggatccaccacagtcggctgaaaccggcagcccaggacaagtggaccagccagcaggacccagaccatccgacccggctgatcctgcgacgagaccgagttgccgctgagagacgacgacagccctgctctggtcactccggaagctgaccagtctacgcacggctgaagcttgaggagacaacagccctgctctagtcaccccggaagctgactagtctacgcacggccgaagcttgagtantcatcaagnaagtaaatgtggttagaaatcttaagnccagtagttttccttgtaatactaattgttttactattgttctgtcgctttgctcaacctcctcccccgggtaaagacctcttctgtccntgctgggtataaanatgctactctttacttnnttgttccggcgnncgnngctacnnntttcctcctnattatgncactccccttanccgtgtcagaagaagagcccatagaagggtgcccccactgcacncacactacntggtcagggagcacnataactagaaccctgttgtaccatacttattatgagtgtacagggacccgcctaggaacttgtactcacaaccagacgacctactcagtctgtgacccaggaaatggccagccttatatatgttatgaccccaagtccttacctgggacctggtttgaaattcatgtgggtcaaaagaaggaaaccttctaaaccaaaccaaggtctctccctcccacgggggggctatntccttgtactttgatgtttgccagntaacatccatgggctcaacctttcccgtaatctctagttccgnagagtactatagnagctgccacaaaaatatatgcgcaccccctgcttgctccgccaggtccccagaaacagcttgctgggactgcacaatcggtccnctgacccgcaatcaccggggccagtcatgcttaccaaaatgccagcaaaaccagattgtaagacaagcacttgcaatcctgtaaatctcaccatcttaaagccggatctgcccatatggactacaggtttannngcaccccgggantacaagtcagcggtcagaaaacngacccagaagcctatttatatattatcaagaaanctcggacccgtccgcccganaattccagtctttaagtcattctntgagcatatnaaccagaagttgccngagccccctcctttgccagaaacctattcgncgcgnnncgtttgcntgctcagctggctgaaaacatngccggcagcctaggcgtctcctcatgttatgtttgtggagggaccaacatgggagaccaatggccntgggaagcaaaagagttaatgccccaagataacttcactctactgnctcttcccccgaaccgacgcccacaagttcgagcgtctggctcttaaaaacctctattatcgggagatnctgcgttgctcgctggggaaaagcctttacagacccagtaggagaattaacctgcctaggacagcaatattacaatgaaacactaggaaaaactttatggcggggcaaaaataattccaaatcgccccanccaagcccattctcccgtttcccttctttaaaccactcttggtaccaacttgaagctccaaatacctggcaggcgccctctggcctctactggatctgtgggccacgggcatatcggcagttgccggctaaatggacaggggcctgtgtactnggaacaattaggccgtctttcttcctaatcccnctgnaacagggagaagctttagggtaccccgtctatgatgaaactaaaaggagaaacaaaaagagacataaccataggaaattggaaagacaatnaatggccccctgaaagaataatccaatactatgggccagccacctgggcagaagatgggncgtggggataccgcacccctatttacatgctcaaccgcatcataaggttgcaggcagtgcttgaaatcatcactaatganacngcaaangccttagatctgctggcccagcaagccacaaaaatgaggaacgctatctatcaaaatagactggctttagactacctcctagcccaggaaggaggagtatgtggaaagttcaatctaactaattgctgcctagaaatcgatgacaacggaaaggccattangganataactgcaagaatncgaaaattagcccatgttccagtccagacttggaaagggtggtctccagattccctcttcggaggctggttttcatccttcggaggattcaagaccttaataggagtagttctggccatactaggagnctgcctaatactcccttgtctcttacccctccttgttagaagcattcaatcagccatagaggcnnttgtagccaggcaaactaccactcagctaatggctctaactaaatatcaacctttgccaaangaagaaaactgcctcttcatgaagaattaagtaatagtgatgctttctattaaacttcatttataaaaagcatcaaaggggggaa

SEQ ID NO: 20 - DNA, 7202 bp, Homo sapiens
  source 1..7202: mol_type = genomic DNA
agtggcgtccgaacacagggacttcgaggacgtgaacgaagaaggtctgctggagcagaggaactgaaattgacaaggcgaacggggaccccgggacgagtctgccggcagcggatataaggtcagtgccctaaagaggtactgggagcaatataaggtcagtgccctaaagaggtactgggaacgggaagttttctgaatcagnggtaacatggggcagaatttgtctattgaagaaaaacattatcgtgcagttgcttaaagttctgttgagacagtctggngctcaggttagttcncagacactaactaagatgccgcaggaggttattacgcataacccatggtttccacaggcaggcactcttgatgtggaaaattgggacagagcaggagaaggattaaaacgggctcatcaaaaaggtcttaaagttgattcttctgttttctccacttggagtttagttcgtactgtncttctgccattatctccttnttattctgcnggacagcaggagtcatgttctgagtctaaaaatctgaaagaatctgttgtcccacccacagctccaattgaaaataaaaaacaggagagggaggataaaaattggcctataccgcctcctccagttgcagaaacatctgtaccgcctccttcggtagcagaaatagagaccccaatacaaagaattttacgctctgctgccatagctggagagcccttaggacctntgcgcttttcctatttccgtaaggcctgatccaaataatccacagcagnttattcatgaacacaccccactagagtttaagttgttgaaggaattaaaagctaagtgtggtnaataatggcgtacagagcccattcactttaggattgctagaatctgtgtttggtgctatgcgtcttctaccctttgatgtaaaacacttggcncgaacttgcttgtctgctagtgcatatctgacatggaatttaaattggcaagaaatgtgtgcagaccaggctagacagaaccgtgctgctggacacggagacattacagaggatatgctgttaggtaatggcccttnattcagacctggaacgtcaaatggcactcccagacgctgcttatcagcagtgtgcacaggccgctaaacgcgcctgggccacaattcctgaagagggagtcccagtacaatcctttttacatatcatgcaagggtcgcaggaaccctatgcgcaatttcttgcaagattacaagaggcagtgaagcgtcagattcctcataccgctgccgcagaaatgctaaccttaactctagcttttgagaatgcaaacgcggattgtaaacgtgcactggcacctgtgaggtgtaaaaaaacttgggaaattttctcagagcttgtcaggatgtaggaactgagcttcatcgctctgcaatgttagcncaagcaatggctaatttagcagttgacaaatctaaaaggagccaagggtcaaaccctaaaatgggaaaatgttataattgtggaaaaactggacattttaaaaaggaatgccgccagatctcaggacagaaaggaccttacaatgcagtnccccccacccccgcgnnccagcggaaaaaaacgccaggactttgtcctcgctgtaacaaaggaaatcactgggctaatcagtgccgctcaaaatttcatcagaatggcacccccctgtcgggaaacgagangggggcctggacccgggcccctcaaacaatgagggcattcccagtccagaccacaaccccgtttcagggatgggttcccggaggnacattgattccctcaccccaggaacaccaggaagtgcaggattagatctccccgccagagaaagaattacgttagttggnggagacaaacccaccaaagttcccactggcatttggggacctttaccagcaggatacatgggactaattttaggcaaaagccgccttaacttgcaaggcattactgtagtcccaggagtngttgactccgattatgaaggagaaattcaagtagttttaatgtcacaagatctttgggtttttgaaccgggagaatatattgctcaattattgcttattccctgcaaattacacccttctccacgaaaggagaaacgaggaaataaagggtttgggagcacaactacatgggaaatctatctatccncaacccatagcctctaatagacccacctgtgtagtacaaattaaaggaaagaaattttatgggcttatggatacgggagctgatgtgtcagtaatatctagnaacgactggcccccatcctggcccctgcgattaacttctacatccctagtgggagtaggaacagctcaaagtgttcaacagagtgctgagattttaccttgtcttggtccggatggacagtcatgtacttttcagccttatgttgcaaatatagctatcaatttatggggtcgagacttacttacagcatgggatatgagacttacaaatgaaaactttgataacccaggatttaaaatgttgaaggacatgggatatcagagtggaaaaggtttagggaaattcctacaaggaaaccctaacccgatatcagtaactggaaaaacagatagaaaagggctaggacgtcaggatttctgacgggggtcattgatatttctcctccgcccactgccttaccattagaatggctnagtgacaaacctgtgtgggtggatcaatggcccctancacaggagaagctagntcaacttcatcngctagtaaaagagcaattggatgcaggacatatagagaagagttncagcccctggaattcaccggtgtttgttattccaaaaaagtccggaagatggtgactgctgcatgatttgagagctattaatgcacaaattaaaccgatgggtgcattacagcaaggtctgccatccccagcggccattccaagagactggcctctcgtagtaatagatcttaaggattgtttctttactataccnttacacgagaaggataagcctcgatttgccttctctgtgccttctattaatcaaagagaacctgtttctcgttatcaatggaaagttttaccccaaggcatgcttaacagtcctacgctatgtcagcattttgtaggacgggcattaaaggagcctcggaatatgtttcccactgcttacatcattcattntatggatgatattcttttggccgctcctacagatcaaatcttacatcagttattcagagaaacaaagcgggctttgactaaatggaatctcaaaatngctccagagaaggtgcaaacaacttccccataccantacttaggaactattgttacggagagaagtgtacggcctcagaaagtagttctccgtaaagacaggttacagactttaaatgattttcaacaattattaggggatattaattggctgcgcccgatgctaggtattgctacctatcaacttacacatctttaccaaaccctgcaaggagattcttcnttaaattccccgcggcaactnactaaagaggcagaagccgagttacggcttgtagagcagatgcttcagcagagacatgcctcncggctacagccgcaaaaacctttgcttttgtttattcttcctaccccccactctccaacaggacttttgggccagttcatagacaagtctgtaacagtaatagaatggctctttctacctaatcagtcaaaaccttgcaagtttatctttctttaattacacaaattgtgactatgggcaggcataggtcaaaaatgcttacgggatatgatccngacaaaattattgttcccttagactcccagcaacaggccgcagcntgggaaatgtcgactgcntggcaaatcgctttcgcagacttcgtgggaataatagataaccactatccctcagacaaaattttgcagttttataaagtccattctttcattcttcctgtgattactcatcacaagcctattccaggtggacagacttattttactgatggctcttccaaaggtcgtgcagctatctatggacctaaacatactcaaacaataatgacctctggggtttcagctcaacgctcagagctaattgcagtcattcaggttttacagctcacagcttcagatcctatcaacattgtctgtgattcagcttatgttgtaaatgtagccagtcgcatagaaactgctacaattaaaagtacactagacccagaactgcttaatttgtttctaagacttcanacagctattcgctctcgtgcagctccttttcatatttctcatattcgctctcacacacaacttcctggaccactatctctaggtaatgatagagcagataaactgattggttctgtgtttcagcaagctcaagcntctcnatgcgctactgcaccaaaacacctccgcccttactcgcatgtttcatctgcctcgcagccaagctagggctatngtacaagcctgtcctacttgccagcatgtccctggngccgcacctgtagaaggntgtaacccacgaggtttggctccaaatgaaatctggcaaatggatgttacacacatagcagcctttggcaagcttagctatgttctgtganctatagacacttattctcatatgctgcatgctacatgccaaacaggtgagacagctggtcatgtacggcgacattgtctgtcatcatttgctcatatggggatacctaaacaattaaaaactgacaatggacccgcttatactagtcatgcttttcaaaatttcttacagctttgggctataacccataaaacaggaattccttataatcctagaggacaaggcattatagagcgggcacatcaaacattacaacgcatgttgaaaaaacaaaaagggnggtataggaggccaactaccacctcaatcaaaactacatttagccttatttactttaaattttttngactcctggtacggatggtaagactccagcagaaagacattggcaagtgttagaggaaaagaggaaagtttatccgaaagtgttatggaaatccccggaagaagngacaatggaaaggtccggtggatttactgacgtggggangagggtatgcttgtgtttttacaggagatggacaaaccgtgtgggtgccctcaaggtgcgtgcgaccatggaacgggagactggaggaacccagggtggccaaccatgggcccggtccctccggtacgagccatgagccagctgagcctgagtgcaaagacggagagaaggccgaccggagtcacgacgacatcaacccccataacctggggacaactcaagaaaaccacgcaggaagctgagaaactactggagcgtcagggncaggcaaaaacccctgattccatgttcttggccatgttagccataatgtcctgtgcggtatgttttccctgtgcagaggcaaaaacatattgggcatatgttcccaatcccccagcagtacgacctgtactttggagtgacactcctcctgagatttatcatgatcagggagcgtgggctccaggacccctaactcccctgacantagaacagttagactctcagaacaatgtcatcaattataccgctccactggaaggactccctttgtgtatcaccacaaagacgtcgctcagccgtagctgtcttacaattcaagctcaagcatggttgagtcactatggaaaagtcatgtacttattaggtcttggttctattaatgtaactggtgtgctaaccaaccattcccggcccaatcgccctaattgtgctgactatacggaatggattcccttcaatagttcctaccccccctcncgtggacccagtgtcttggcccactggctagaaaacaatctatgttaactggagacattgtggattggggacctaaaggtcaattagatggaaaagatgaaaatcagaaatcatggcacaaacttcgctggcattggtggcaagcttttaatgcttcttctttatacnacaccgggatccaatcccagtctgccgcccagattgcttggcatggagcaggctttagcccgcctcttcctcagtggcattatctagggaggaaaggaccaattcaagagacgatatggaaggcagcactcccatttacgaatggagcatctgggttngggatactatccaataatagcaatagtaagcgacacagtcttaatgttacatttgtaaagaatatcaccactcaatttacggtttgtgtttttaatccttatgtctttttggcagctaagaaggaccagctccaggtaaacaatacccaattgacctgtaaatcttgccagttatatcactgcattaatcatagcacattgcaaacacataatatctctactttgatgattttgggtcgcatccctgggctatggattcctgttaatctgtccgagccttgggctgccacacctgctttgcattttgtgaaacttcttctaactcagcttactcatcgtgtccgtagagccttaggcatgataatttttgctattgtttccttggtcacactaataacttctgttgtgatgtcctctgtagctttgcatagttctgttcaaacagctcagtacgtggagaactggacgcgcacagccgaccaagcgtggctacttcagaataaaattaacactgagttacaaactgaagtggcaatgttgaaatccacggttctatggttaggggaacaagtacaaagcttgcagttgcagcagcaattgcgttgtcattttaatcacactcatatttgtgtaaccaacttagaatataaccaaagtgagtatccgtgggaccttgtgaaagcccatttgcagggagctttcacatccaacatcacctttgatattggtgaattacaaaacaaaattcttgatttaaataggcaaactcaagagtttcagccttctttagaagactggaccgaattccagcaaggcctggagagcctcaacccttggacctatctaaggcaccacattaacatcttatatgtagttcttggaataatgttgttttgtctctgtcttctgttcatagtctgtaaaatcggatggaccgccaatcggagaatgagagctgcccagcctggccttacattctttcaattaatncataaacagaaagggggata

SEQ ID NO: 21 - DNA, 5654 bp, Homo sapiens
  source 1..5654: mol_type = genomic DNA
gattttggtaccaggagtggttctagaggaacagaatattaaggatggagttctttcgttggttttggggtttctggagttggctgcttaatatgattagacccaaaaatgctaaggactctacttctaatagtatggagaacactgatagtccttggcgtgaactgtttagagagttatgcaaaataaatgcatttgacactcctgattcaccgctcgtgagaggcaaggagtttagtgactctatacataatacctttgaccatatgtggagaaccaaggaacataatgaagctggttggttgctcctaagttcagtggacaaagtgatgaaagaaaatgatgaactcagggattctatctcccggcttcagaagcagatactgagcctcaaatctgctaagattgccctgagtgagagtcttatctcctgtagagaaagagctgaaattgtggaaaaacagacacaagctcttatcatgcgagtggctgacctgcaacgaaaggtgcatgcacagcctcgccaggtgtctactgttaaagtgagggcattgattggaaaagaatgggaccctgcaacttggaatggggacgtgtgggaggaccctgatgaagctggggacactgagtttgtaaactctgatgaaccttttttgccagaagaaacagcttccccatccccagtagtggcaacatcccctccccgacccatgctgccatcagcctttccacctttgtctgaggagataaaccctgcgctgcctgaggcaacagtgatggcctcccctgaggcagttgccaggcaagataatgttgattctcctcaggagccacccccaacacccctgtttgcttctagacctataactagactaaagtcccggcgggcccctagaggtgaggttgagagtgtgacccatgaggaggtgcgctacactcgaaaagaactgcttgagttttctaatttatataaacagaaatctggagaacaggcatgggaatggatattaagggtgtgggataatggtggaaggaacatagagttggatcaggctgaatttattgatttgggcccactaagtagggactctgcatttaatgttgcagctcggggagttaaaaaaggttctaatagtttatttgcttggttagctgaaatatggattaaaagatggcccactgtgagcgagctggaaatgcctgatctcccttggtttaatgtagaggaagggatccaaaggcttagggagattgggatggtggagtggattagtcactttagacctactcatcccagctgggagggtccagaagatatacccttgaccaatgccttgcgaaatagatttgtgagggcagcacctgcatctttgaagagccctgtaattgctcttctctgtatgtcagatctaacagtgggaaccgcagtcactcaactacaaaatttaaatacaatgggaataattggatcccgaggtggcaggggccaagtggcggcactcaaccgtcaaaggcaaggtgggcgtagctaccgtaatggacagcagaggcaaagcggcaatcagaatagtctgactcgtgtagagctctggcattggctaattaatcacggtgttcctagaagtgaaattgataggaagcctactgcattcctacttaatttatataagcagaaaacttctaggtcgaatggacaaaagactaatttgaattataaaaacagagaatcacggcccctcaatcaatttccagacttgagccagtttacagacccagaaccccttgaatgaaggggaggccgggtccccttgaggaaggaccccactacactaccgacaatttatgcngtgaatctttctcccatccttccccaaggagacctccggccttttaccagggtaactgtgcactggggaaagggaaatgatcagacatttcggggactactggacactggctctgagctgacgttgattccaggggacccaaaacgtcattgtggtcctccagttaaagtaggggcttatggaggtcaggtaattaatggagttttagctcaggtccgacttacagtgggtccagtgggtccccggactcatcctgtggtcatttccccagtgccagaatgcataattggcatagacatacttagcagctggcagaacccccacattggctccctgactggtagggtgagggctattatggtgggaaaggccaaatggaagccattagagctgcctctacctagaaaaatagtaaatcaaaaacaatatcgcatccctggagggattgcggagattagtgccaccatcaaggacttgaaagacgcaggggtggtgattcccaccacatccccgttcaactctcccatttggcctgtgcagaagacagatggatcttggagaatgacagtggattatcgtaagcttaaccaagtggtgactccaattgcagctgctgtaccagatgtggtttcattgcttgagcaaattaacacatctcctggtacctggtatgcagccattgacttggcaaatgcctttttctccattcctgtccataaggcccaccagaagcaatttgccttcagctggcaaggccagcaatatacctttactgtcctacctcaggggtatatcaactctccggctttgtgtcataatcttattcggagagaccttgatcgcttttcgcttccgcaagatatcacactggtccattacattgatgacattatgctgattggatccagtgagcaagaagtagcaaacacactggacttattggtgagacatttgcgtgccagaggatgggaaataaatccgactaaaattcagggaccttctacctcagtaaaatttctaggggtccagtggtgtggggcctgtcgagatattccttctaaggtgaaggataagttgctgcatttggcccctcctacaaccaagaaagaggcacaacgcctagtgggcctatttggattttggaggcaacacattcctcatttgggtgtgttactccggcccatttatcgagtgacccgaaaggctgccagttttgagtggggtccagaacaggagaaggctctgcaacaggtccaggctgctgtgcaagctgctctgccacttgggccatatgacccagcagatccaatggtgcttgaggtgtcagtggcagatagggatgctgtttggagcctttggcaggcccccataggtgaatcacagcggaggcctctaggattttggagcaaggccctgccatcttctgcagataactactctccttttgagagacagctcttggcctgttactgggctttggtggaaactgaacgtttgactatgggtcatcaagtcaccatgcgacctgaactgcctatcatgaactgggtgctttctgacccatctagccataaagtgggtcgtgcacagcagcattccatcatcaaatggaagtggtatatacgtgatcgggctcgagcaggtcctgaaggcacaagtaagttacatgaggaagtggctcaaatgcccatggtctccactcctgccaccctgccttctctcccccagcctgcaccgatggcctcatggggagttccctatgatcagttgacagaggaagagaagactagggcctggttcacagatggttctgcacgatatgcaggcaccacccgaaagtggacagctgcagcactacagcccctttctaggacatccctgaaggacagcggtgaagggaaatcttcccagtgggcagaacttcgagcagtgcacctggttgtgcactttgcatggaaggagaaatggccagatgtgcgattatatactgattcatgggctgtagccaatggtttggctggatggtcagggacttggaagaagcatgattggaaaattggtgacaaagaaatttggggaagaggtatgtggatggacctctctgagtggtcaaaaactgtgaagatatttgtatcccatgtgagtgctcaccaacgggtgacctcagcagaggaggattttaataatcaagtggataggatgacccgttctgtggacaccactcagcctctttccccagccacccctgtcatcgcccaatgggcccatgaacaaagtggccatggtggcagggatggaggttacgcatgggctcagcaacatggacttccactcaccaaggctgacctggctacggccactgctgagtgcccaatttgccagcagcagagaccaacactgagccctcgatatggcaccattcctcggggtgatcagccagctacctggtggcaggttgattatattggacctcttccatcatggaaagggcagaggtttgtcctcactggaatagacacttactccggatatgggtttgcctatcctgcacgcaatgcttctgccaagactaccatccgtggactcacggaatgccttatccaccgtcatggtattccacacagcattgcctctgaccaaggcactcactttacggctaaagaagtgcggcagtgggctcatgctcatggaattcactggtcttaccatgttccccatcatcctgaagcagctggattgatagaacggtggaatggccttttgaagtcacaattacaacgccaactaggtgacaatactttgcagggctggggcaaagttctccagaaggccgtgtatgctctgaatcagcgtccaatatatggtactgtttctcccatagccaggattcacgggtccaggaatcaaggggtggaagtggaagtggcaccactcaccatcacccctagtgatccactagcaaaatttttgcttcctgttcccgcgacattacgttctgctggcctagaggtcttagttccagagggaggaacgctgccaccaggagacacaacaacgattccattaaactggaagttaagattgccacctggacactttgggctcctcctacctttaagtcaacaggctaagaagggagttacagtgttggctggggtgattgacccggactatcaagatgaaatcagtctactactccacaacggaggtaaggaagagtatgcatggaatacaggagatccattagggcgtctcttagtattaccatgccctgtgattaaggtcaatgggaaactacaacagcccaatccaggcaggactacaaatggcccagacccttcaggaatgaaggtttgggtcactccaccaggaaaaaaaccacgacctgctgaggtgcttgctgaaggcaaagggaatacagaatgggtagtagaagaaggtagtcatcaataccagctacgaccacgtgaccagctgcagaaacgaggactgtaattgtcatgagtatttcctccttcttttgttaaaaacatgtttgtgcatgtatacacttgtactaagaaaatatcttcattttatttcctttctcctttatcatgtgacataagatttattgacttcatatcagcatttaagtattgttaactttatgtaatagtatttgggttggggattggtgcgtttccggttgtacgaaggatagttgtattatgttaggcgtaattatgaccttattattgtctttatttgaagattatgtatgatctcaggagatgtgtatgggttcaagttgacaaggggtggact

SEQ ID NO: 22 - DNA, 6047 bp, Homo sapiens
  source 1..6047: mol_type = genomic DNA
atagtcggcaggatcccgaggtgagtgagccttcggcccccgatgatcccgggtcggccatgtggccgcagcatgggctgtggtacccggtggcagccgtgctgctcggatgggccccggtggaaacntgggcggcggtggacgggtcccccgcgagcgtggagaaggcgctgaagcagctggaagcgcagagcaccgagaaggagcgcgcctttgccggcagagtcggatgggcgtttttgactgcgctacgggaagtacacgcccagtccctgcgggatgcagcgcaggtaagggacctccaggcgcaagcggggcgcctggaggcccggntacacagctcggaanaaganttgggggtaagggacctccaggcgcaggcggggcgcctggaggcccggntacacagcttggaanaggaattagaggctgccgtnagtgcgggcctgggcccgtcgtcccggccggagacccccgctcggtctgataccgaggaggaagaaccccccgttgcgggctcgcccagtggtccgtcagaaggtagancatgaacagccgntggggccccaagggcgggctcagggaccccccaccgtgacggaacacacttcatatagtgcctataccccaactgagttgcgggaattaggcaagcagtgccggcagcgtccgggggaacccctgcccgcctggatgcttcgtctntgggacgagggagcngacagtatttcctgctccgcctctgagatggaaaagctggcctctatcacgactcacccctccctccgtcagcggttgcaggtgagcaggcggttggcacaagggcaaggtgaccacaccctgattgagtggctgatggcagccatacggactgtgtggaacgacgccggngaaataccagaaactgtgagtaaatggcagtcgtataccgatttggtgcaagtnatccgggagatgggtatgcggcaggctatgtttgatctgaatacccgggggccagatgatgaacgctttacctcccacatgagggatctcgtgttgggctctgcgcccccgagtgcctttggctccctagccgctgtcctcaccccgtacgtggggcgccgcatacatgaagtgactactgctatggcggccctcggggaagcagaaggccatcggcgggaccggggggtccgcgccataaagaaggggaaggtgccccncccgcagggggccaccccacggganaaaagggggccccagcgggtgacncgcncgcagatgtggatngatttgatttcggccggggttgaccgagagaaaattgataggcagcccaatgaagtgctgttaactttgtggaggcaattgtccccggagcagcaattccggaaaatgcccaagggggggnaggacantgctgcgcgacccagtcccgcccgggcgctccagctcaaggactacttgcagncgggcggagatgtagagccttttctgtttgattagggaactggccgaggtgcccggcttggggggacaccggacgaccggaggccacatgtggaattggcaatccactggtcccccaccaatgtacagcgggtgctggcgctggtagatactggcgcagattgcagcctcgtttatgggaacccggataagtttccgggcaaagctgcatacattgacggttatggaggccggtcagtgaaagtgaaacctgtatctctgcaccttggcatcggccgcttggctccccgcttatacactgtgtatgtctctcccatacctgaatacattctgggggtggacgttttacacggcttggncttacaaaccacggccggagaattcagactccgagtncgtgtggtgaagccggtgctgcgtggacatacgcatcaccagccccaggtcctgccacaaccccgacgggttacttccacccgtcaataccgtttgccgggtgggcatacggagataactgagacnattaagaagctggaggaggtgcagatagtgcgtggcacccacagcccctacaattctccggtatggccagtcagaaagcctgatggaacttggcggatgacggtggactatcgggaactgaataaagtaacaccccctttgcatgcagctgtgccgtctatcacggatttgatggaccgcttgacgacggaactgggacagtaccactatgtggtggacttggccaatgcattcttctccatcgacattgctccagagagccaggaacagtttgccttcacgtgggaggggcgacaatggactttcacagtgttgccgcagggctatgtgcatagccccaccatatgtcatggtcttgttgccacggatttagccgcctggnaatgtccagaaggggtccgcctattccattatattgatgatattatgttaacctctgattctcttgcagatttagaagcggcggcgcccctcttgcggcaacatttggcagcatgcggttgggccgtcaacgaatccaaggtccaagggcctggattgtctgccaaattcttgggagttatctggtcgggtaagacgaaggccataccagaggccatcattgataaaattcaggcatatccccggcccaccacggtgaggcagctgcagacttttgtgggcctcctgggatattggcgggcatttgtgccccatttggctcaaatgataaaaccgttgtatcggttaacaaagaagggagctacctgggattgggatgatgcggctgagacngccttcctggcagccaagcgggctattcagcaggcacaagccctacgggtagttgaccggggcgcccatttgagctggatgtgcatgtgaccacagatggtttcggctggggcctatggcagcgcacggagcgcttgagaacgccagtaggcttttggtcccaactatggaagggagctgagctccggtattcnttgatagagaagcagttagcagctgcatatgccgcccttcaggctcgtgagagcgtgacaggacgggctacagtcgtcgtgcggacgacttacccaatagcgggatgggtgcgttcatgggtaacgaccccccggactgggacggcgcagacatccactttagcaaagtggggcgcctacttggagcagcggagtacgctgagtacaagtcccttagcagcagagttgcaagaggtcttgggacctgtagtcctaatgcaagataaggccatggggcctgaggcacccctagaccctgagccttcaccgtttaaggaagggcgtccccccattcccgatggggcatggtacacagatgggtctagccggggtgctactgctgcctggaccgctgtcgcagtccagcctagtactgacaccatatggtttganaccgggtgtggacaaagtagccaatgggctgaactcagagcagtgtggatggtgatcaccaaggaggtgacacctatggtaatctgcaccgatagctgggcagtttatcgaggcttaacttgtggttaactacctggaagttacagaantggctagttggccaccggcccatntggggccaagccatgtggcaagacctatgggaaatgggtcatcagaaagangtaactatttatcatgtgtcaggccatatgcctttggccacccccggcaatgatgaggcagatgccttggccaaggtccgatggttagagtcggcacctacacgagatgtggccttgtggctacaccggaaactgggacatgcggggggtaaactgatgcaacaggtcaataagcgttggggtctgtccctgcccacgcaagacatttgggaggcttgtcagaaatgcccggcatgtgctcaggcataccctaaacggaggcagctgcccagtgttacacaacaagtgacgatagggcgggtgcccttgaccaggtggcaagtagactacatcgggccgctgccgaagtcgcaggggtatacgcatgcgctgacggctgtggacacggccacaggcctgttgttcgcctacccttgcagggtggccgaccaacagcacaccatccgggccctgcaacacttatgtgccctgtatggtcgccctctggccgttgagagtgataggggaacacatttcactggacaacaggtacaacagtgggcacaacagatggacataaagtggggattccatgtgccatacaacccgcaagccgcgggtatgattgagcgatataacgggctcttgaagaatgggttacgcttgcatgtcacncccccgtctttgcggggctggagttccaggctggacctggtgctccaaaccttgaatgaacggccacggaaaggcggcccggccccggtggaggcnttgttacaccgggccgccgcccccattcagttgcagatacacaccaaggatgacctcctccgaccaggtatggggacgaacggtaacctgttgttgcctgccccaacgcccctgaaggcaggggaacagaaaacctggctttggccatggaccctccaagccccccactgccggtggttggccatcgtagccccctggggggagggcctacagtatgacttgcatgtcactccttgggtattcaatgcgtggcccccgcggttgaccgttcgtaggggaacggccagggaaggaaccctcctccgggggacatatgtactgtctgtgtggcctattatgagctcccccgtgactttggcacggatacaggacccaaaggaaccatggggagctgagaaggtgtggtaccatcgcccagggcagaagcccttggcggctgcattgttatccagggatgaaaggttagcctgtattttgcctgagggacgtgatttacccctgttagtacctgtgcctgctctgtcgtttcggccgtaggttgacatgctccaacngcattgtggactgggcccacacctacgctgaggtgaccaatgtttccaactgttggatctgcaccgcccttccagcagcagctgcggacggcttgccctggcacatacatccagcntctgcggagaactggacatggctggagacttggggtcccatggccgacgcctggaatgcaacgcggcaagctttggacagggggcgccgcaagacccatggcancgcccgccccctggctggcccgtagcgtctatgatgggtggggctggctagtgggggaacacgtggtgcccccancgcaggtaccacggtgcatagagcaacactggggtaacgccactgtgggatggntgcccgccgcggcctgtgcaaacataacacgtgtcaccacaccgaaggtgtggtggaacaagcggccccaccaaggccgggccccgatggactttgtgccccctgggagtttatgggtctgtggggacacagggtggccntacctgccagcgaactggactggacgttgtacctgggggtggccttatgtgcctgccactgttctccccacattgcccagacgcccgcataactgggaggcgctacgctcccggtttttgcgagtgcggcgagccccctggtggttctaccccttggcaatgactatccctggagcgggtgtcatnactgtagaagcgcaagttacngcccttgcagagcacaccgctcgggccctgaattacacccgagtngccctcctcctgttaacngatgaggttgatcagatcagaaaggtggtgctgcaaaaccggatggccttagacatagtaactgctgcccaaggnggcacctgtgcccttttaggaacacaatgttgtacctttatccctgacaatcggcagaacataacagcagccctgcaaggggtntcacgggagattaaggcggtcgagagccttactgacgaccccctgcagagatggtgggcatccctgggctctggcctacgctgggccctaatagtcataggtagcatagcngggatnctagtagtgagctgttgctctctgtattgttgctgtggcctatgggtccagggctccgccctatgggcacgtgtccccgccnagaggacnccctcggcctagggggtgga

SEQ ID NO: 23 - DNA, 609 bp, Homo sapiens
  source 1..609: mol_type = genomic DNA
tgttaaatacagtgagttccgagtttctcttcaaagaaccagtatgtcagtatgttcagctcccctgttctttgttctccattttaaagtttaacttccgtnnnttcgnctcncctgctccttgttctccattnnaaantttnncccgccagttctaatcagtagttcacatctgttcccctggtcacctgctccgtcctgagtcacccctggtcacctgctctgacctgagtcacccttagtcacctgttctgtaaccgtccttcccgccaaactactcaccccgccactccggctcgtacccctgctctctttaaaatagccaatcggaattagcttagactgtgcggtccaaccctagccaataggggaacgacacagcagtaggggctacctgcgtcaggaataagaaccccttcccctcccttgtccaggtgtgctctcgccattgctccatccgcgagncgcacccttctatagaagtaaaattgccttgctgagaaaattaaatttatgttcgagtgctatttcttttgcggcaccgaaacaagcattaaatttatgttcaagtgctatttctttcgcggcaccgaaactttacgtataaca

SEQ ID NO: 24 - DNA, 633 bp, Homo sapiens
  source 1..633: mol_type = genomic DNA
tgtagaaagtaaaaagtttcctcttcaaagtttcccttcttgttaaagaataaatcataagtgttagaaataatagtttcttttaaagactaacttccttcaagcctccttgctttgtgctaataactctttgttaagccctatcctatgtagctgttagacatagggaataagtacattctatgtccttgtactttaaccaagatatttgtgctggacgtgctcacaggcacgtcccagctcgcagcctatgccccttccttatttggaaatgttattgcttctctaagtcctttcgtaagcaacttcctcttttcctttgttctcccttgcntttacctatttaggaaagttttaagttattagccaatcgggtntcagtttagattgtgaggtccggctccagccaatggagacaggacacagcagcaaggacaaactgcgtaagggataaanattgcttccctcctttgttcaggtgtgctctcgccattgttccatctgcgangagcaccctttctgcagaaagtaaagattgccttgctgagagaactttttgtctgagtgctgacttttctttgcggcaccgaggaacaagcattctgtttctaaataaacattttacttataaca

SEQ ID NO: 25 - DNA, 1007 bp, Homo sapiens
  source 1..1007: mol_type = genomic DNA
tgtgggcggcaagccacccaggcgccgaggcaagagaccgagggcacgagctgttccagtataataaaatatataaaacaagaatagttataccagatatagatcttagatatgattatatatgaatatcattaatcattagtttgtagcaattactctttattccaatattataataatcctcgctctataatcataacctaggaaaaaccaggccatacagagataggagctgaggggacatagtgaggagtgaccagaagacaagagtgcgagccttctgttatgcccggacagggccaccagagggctccttggtctagcggtaacgccagcgtctgggaagacgcccgttgccaggcggaccgtggtctagcggtagcgtnagtgtcaaggaaaaacacccgctacttagcagaccgggaaagggagtctccctttccccgggggagtttagagaagactctgctcctccacctcttgtggagggcctgacatcagtcaggcccgcccgcagttatccggaggcctaaccgtctccctgtgatgctgtgcttcagtggtcacgctcctagtccgccttcatgttccatcctgtacacctggctctgccttctagatagcagtagcaaattagtgaaagtactaaaagtctctgatatgcagaaataatggcgtaagctgtctctctctctctcctctctctctctgcctcggctgccaggcagggaagggccccctgtccagtggacacgtgacccacgtgaccttacctatcattggagatggctcacactccttaccctgcccctttgccttgtatccaataaatancagcgcagccnggcattcggggccactaccggtctccgcgccttggtggtagtggtcccccgggcccagctgtcttttcttttatctctttgtcttgtgtctttatttctacactctctcgtctccgcacacggggagaaaancccaccgaccctgtggggctggtccctaca

SEQ ID NO: 26 - DNA, 438 bp, Homo sapiens; identical to SEQ ID NO: 10
  source 1..438: mol_type = genomic DNA

SEQ ID NO: 27 - DNA, 451 bp, Homo sapiens
  source 1..451: mol_type = genomic DNA
tgtggtggacgctgtgatgcgccgcccagatcccccttcaggantgaaggacttattcccccagctgctgggagtgctgccggcagacagccctcagctgtcagccctcttcggggattgcctcagctgaagagagccgcctcgcccaaggtcacgcccccttcccggggcagcccgcatccaatgactgatcgatgcgggggtataaaggcccggccccctcgccccaactcgggacaactctgaagggccatcccagctccagagctccccgtggggtcggctgaggccttcgttgggactgcatcgcagctcaacttctccctctgcccaatcctgcttccttcccttcccttccacaggtgttgatcccaagagcactccctaataaacntcctgcacgctaatctccgtctcagagtctgcttcccggggaacccaacctgcgaca

SEQ ID NO: 28 - DNA, 456 bp, Homo sapiens
  source 1..456: mol_type = genomic DNA
tgtagtagatgctgttggtgccccgcccagatcccctttaccgggccggtgcacccatcccccagctgctgtgagtgttggctgctaacggctcacagctgcccccttctccggagaattgccctcggccgacgggagccgcctcgcctgggaggttaccccccaccgggcagcccgcagccaatgactgactgatacgggggtacaaaaggccggcccccttgcctcaaggtgggacaactcactctgtggtgcaattcatgctccagagctccccgtgggatcaggctgaggctagacttcagctgagaccacatccttgcttagcttcttcccctgccctatcctgcttccctcactcccttacaggtttctcctgagagcactccctcaataaatcacttgcacaagaatccccgtctcaggctctgcttctagggaacccgacctaagaca

SEQ ID NO: 29 - DNA, 495 bp, Homo sapiens
  source 1..495: mol_type = genomic DNA
tgtagcggacactgtcggtgcccgcccagatcccctcggatcccttttaccgtttctgtgcgccccttccccagcttctgtgtgcttttgcttctaacggcccgcacctgcgactcttttcggaggactgccctcgggctactggagccgctttgcccgcatgcagagagccggaagtgcctgggagtttatgncctctttccctccagggcggcccttagccaatgactgactggtgcgggagtatgaaagcccagctcccttgcctcgagtcgggacaaactctgaggcgtaatttacactccagagctcccctgcgggatcaggctgaagctgggactttgcctgaaatcgcacccttgcttggcttcttccccttccctgtcctgcttccccactcccttaccggtttctcctgggagcacttccttaataaatcacttgcacacgaatcctcgtctcagggtctgcttctggggaacccgacctaagaca

SEQ ID NO: 30 - DNA, 463 bp, Homo sapiens
  source 1..463: mol_type = genomic DNA
tgtggcggccatgaaaatgcgccgctcagatctcctgctgcggggagcatagttgactgacggccccagctgctgcccctctggatccaccaccgcgttcgcgccgaggccacgcttcccncgggctgctcccagccaatgactgagcacggcgggggtactaatgcaggcccattcctgcgagacgcgggactcctctaacgggcgactttggctcgaggactccccatcggcctggccgaaactttcttagaactgcgctgcagtctgagactcttcctacccaatcctccttccttccccctctccttcacaggtgtcagacctgcatcgcggtctgaaggctctccctgcctnctcctgctccctcccctttatccttcacaggcgtttcccccaataaatctcttgcacgtctaatcccgtcttggcgtctgcttctcggaggacccgaactaacaca

SEQ ID NO: 31 - DNA, 480 bp, Homo sapiens
  source 1..480: mol_type = genomic DNA
tgtggcggccatggaggtgcgccgctcagatctcccttcaagagaacctgctgcggggagcgtagttggctgacagcctccagctgccgcaccttcggatccgccgcagcgttcacgccgaggccacgcttcccccgggctgctcccagccaatgactgagcacggcgggggtactagagccgggccattcctgcccgacgcgggactcctctaacgggcaatctttgctcngggactccccatcggcctggccgagactttctcagagctgcgctgcagtctgaggctcttcctacccaatcctccttccttccctctctcctttcacaggtgtcagacctgcatcgcggtctgaaggctctccctgcctactcctgctccctctcccctttatccttcacaggcgtttcccccaataaatctcttgcacgtctaattccgtcttggcgtctgcttctcggaggacccgaactgacaca

SEQ ID NO: 32 - DNA, 462 bp, Homo sapiens
  source 1..462: mol_type = genomic DNA
tgtggcggccatgagaatgcgcctctcagatctccnactgcagggagcgtaattgaccganggccccagctgctgcgctctgaatccatcaccgcgtttgcgccgaggccacgcttcccacgggctgctcccagccaatgactgagcgcggcagggatactaaggcaggcccgttcctgggagacgcgggactcctctgacgggcgactttggctcgaggactccccgncggccttgccgaancttccttagaactgcacngcagtctaagacgcttcctacccaaccttccttccttccctctctccttcacnggggtcagacctgcatcgcggtctgacggctctcccagcctcctccggctccctccccattttccctcacaggcgtttcccctaataaatctcttgcacgtttaatcccgtcttggcgtctgcttctcggaggacccggactaacaca

SEQ ID NO: 33 - DNA, 489 bp, Homo sapiens; identical to SEQ ID NO: 4
  source 1..489: mol_type = genomic DNA

SEQ ID NO: 34 - DNA, 651 bp, Homo sapiens
  source 1..651: mol_type = genomic DNA
tgtatcggacacatcttatgcgcctcctcagattctctcggcctcacctgtctcccggaccctcggccgcttgctctgcctcagccgccgccgcggccgaccgactccgtgcgggcccatgcggttccgccgcctgaggccgaccagccacatgcccggagtctctggctcctcccgccacttccggactcggatgaaacgccacaccgcagggcgtgggatctggcttcccnagcggctgccgaaggggccggatgacgcaacccggaagtgcgggggagttaactccccgtggggcgaactttgaccaatgggagacaggagacgggagggagccgggcagataaattccctctccttccctccctccatggactgttccgaggcacggtttctccgtacagcctgtccggagacgtcccgcgtggccgagcggatgcacctgccgagcgaccggctgtgtctcttcgcggctcgtcgtgaagcngtggccagcncggtaacgcatcaccttgcattgcttcccntccttccctgcctcacttcccttttccctcactctcgctgccctgggattgcacctcccaaataaagcattagcacntaanctttgcctcaggctctgttttctagggaacccgggctaagaca

SEQ ID NO: 35 - DNA, 675 bp, Homo sapiens
  source 1..675: mol_type = genomic DNA
tgtatcggacacaaaccttgtgcgccttctcagattccctcgactgcctccttcttccgaaggtgccgcctcctncccgggccactccgctgccaccgcctttgctgagctccctgagctggctggagtccgagttgaagccccgggcctgcatggccctcagtcccacacgccgagccccatccgcgccgagtcgcccctccacttccggacttggatgaaacgccacaccgcggggcatgggatctggcttcctgagcggccgccgaaggggccggatgacgcaacccggaagtgtaggggagttagctccccgtggggtgaaccttgaccaatgggaaacgggagacgggagggagccgggcagataaattccccctcctttctcccttccgtggactgctccgaggtgtggtttctccttgcagcccttccggagaagtcccgcgtgccgagcgaacacgcctgccgagcgacctgctgtgtctcttcgcggctcgtcgtgaagcggtggccagcgcggtaacgcatcgcatcgcattgcttcgcatctttccttgcctcacttccctttttcctcaccctcaccgccctgggcttgcacctcccaaataaagtgtcagcactttaatccttgcctcaggctctgctttctagaggacccgggctaagaca

SEQ ID NO: 36 - DNA, 572 bp, Homo sapiens
  source 1..572: mol_type = genomic DNA
tgtattggacacaaaccttgtgcgccttctcagattccctcgaccgcctccttctttctctctcttggccggcgccccacccgcgccgggtcgcccctccactttcggacttggatgaaacgccacaccgcggggcatgggatctggcttcctgagcggccgccgaaggggccggatgacgcaacccggaagtgcaggggagttagctccccgtggggtgaaccttgaccaatgggaaacgggagacgggagggagccgggcagataaattccccctcctttctctcttccgtggactactccgaggtgcggttcctccttgcaacccttccggagaagtcccgcgtgccgagcgaacacgcctgctgagcgacctgctgtgtctcttcgcggctcgttgtgaagcggtagccagcgcggtaacgcatcgcatcgcattgcttcgcatctttccttgcctcacttccctttttcctcaccctcgccgccctgggcttgcacctcccaaataaagtgttagcaccttaatccttgcctcaggctctgctttctagaggacccgggctaagaca

SEQ ID NO: 37 - DNA, 538 bp, Homo sapiens; identical to SEQ ID NO: 7
  source 1..538: mol_type = genomic DNA

SEQ ID NO: 38 - DNA, 568 bp, Homo sapiens
  source 1..568: mol_type = genomic DNA
tgtatcggacacctcttgtgcaccacttcanatcctctcggccccacctcttactccagccactgctgcggcgaccagttccgcgcaggcttcgaccagcttcgcgcaggtgcaacctgacagcgcctcgcctcaggcccgcgccgcgtacctctcgcttcctgccccggggcttctctgacgccgcggcgtgggacgcctgcgggaacccgctcggcactcacgcatgcgcaacccggaagtgcgggggagttaacgccccgtggggcaacccttgaccaatgggggacgggagccgatggataaatgcttccccctttcgtcccccgggcggacagttctgagacgcatttcatanggctcctcagaaggtcccggcgggatcgagcaccagtcgcccacagcggtggccaactcgataacgcatccttgtattggctttccctccttccctgtttcactccccctgtccctcactcctgctccctgggatcacttcccaaaataaactacctgcacgcaagcctttgtctcaggctctgctttcgggggaacccaggctaagaca

SEQ ID NO: 39 - DNA, 779 bp, Homo sapiens
  source 1..779: mol_type = genomic DNA
tgttataggagatagaaagaaattatttaggtagacagttagggtaaagngagtccccggcagaaaactttccttctaacaaaaagcagctcagaaatagctccctttctaaccncacgcagttcaaagaaatcacttctcttctaacaaagagcagcctggaagatcaggctgtaaaacacagataagcaactcnggcacagaaggagnggggagtctcctgggtaatcaccaaacttcacacncatacgatgggccccagtaaaaacagtgggccttaataagcacattcctttccctttaggcacactaagatagggaagctggaagcggactggggggggatgcctgcagctgcaagaagatgcctgggaacaggcacggaaactctccctcccagataagcaagacaaagcagcgcggagcagcagactaagagcccgcctgcgtgatcaaggaatggggtgggggctgntagaaaactctgctctatgcagatggcacacctggtcccaaccgaatcttcgggccctangnggataagacaccccctcctcactagccccctcctcactagcccatttataaaaaccctgacatttttactgcagcncggcaacccgttcgggacccctctctgtgacagagagctgttctttcctttcgcctattaaactcctgctccaanctcaccctgtgtgtgtgtgtccgcgncctcgatttccttggccgtgagaccaagaaccttggtatttaccccagacaacgaggctgcttca

SEQ ID NO: 40 - DNA, 635 bp, Homo sapiens
  source 1..635: mol_type = genomic DNA
tgtgaaaataagtaattcaaaatctaagctgttggaactttaaattattttgagccttaaaggaatgtgattatggggcctgagtcacgtgacaggcagctgtaacctaggcagctgtaacctttgtttctctgattatagattangccttcttccttacctacattgttttgtaaaatgttgtaaatgactaaagggcgccagggaagaccccttccctcttcactgttgatcttcattatagattaacttccctcttacctctctcacacaaagacttcatgactatcacattgtcttaagatggaatgttaaatacactcttttaaattggaaaggaaatgaaaacaagctgtaaggaaaagaaaacaagctgtacggaaaagaaaanaaancaaactgtaactaactaattaaattgttgtaactcataaaccagccttgtatagaaaatgttataatcctgctaaatttctttgttttctgcctatataagcaagaccttaacttttaacttcggagcactgaccccatttctctggagtctgtgtttcccggatggccattcccagcttttcgcttgaataaactctttaaaactggattctgatcctttcgattatttcaggttgaca

SEQ ID NO: 41 - DNA, 603 bp, Homo sapiens
  source 1..603: mol_type = genomic DNA
tgaaaccgtccctataaactttataaaattaatcagggaagaagggagggggagaaacgaaaataaaccaagcttgcagcacattcagcattaatcattaggtcagcttgctctctgacctgcttcctcatagttgtttggtgcctattgccccagaatcacgtagaccctgttacaagattatagttccccttaactgctctatagataacaacttgaacattatgaaacgttaagttttccctttgagatattctttcaggtcctgcataccggtgaaactactgacanncgncgtcagctggtctgaaggaccccacgaggagctgactcaccaaagaatgcagtttccacatcctgatgatttcatcccccttaccccgaccaatcaacgaccccaattttccagcccctcgccctccacgatccccttaaaaaccccagcccagaactcctcggggagatggatttgagggtctcctcccatctcctcgctcggcgccctgcgatcattaaactctttctctgctgcaaaccctgctgtctcagtgtaattggtctgttactgcgcagcgggcatacgaacctgttggtcctataaca

SEQ ID NO: 42 - DNA, 515 bp, Homo sapiens
  source 1..515: mol_type = genomic DNA
tgtgctggatattttccgtttgcccctccagatccactctccacccttctccaccctgctctgtgccccgggaggctgacctctatggactgcatcaacgggctcccttgccctctggcttccggttgggttcggccaatgggaggcaccggcaggagatcggagggcgggaggagagtgaggtcggggtatttattcccccggctccctccctgccgggccgcggnttggcagtggctgcgttcctctaccgaaggccacagctcctgtcgggcggccctctcctacagctacagctctctccgggttccggtaaccgctccctccccttgccccttcaggcctaggggtggtaacggctccccgctgttgctagccccggggtgcttcaccatcccttgttggtttcccttaaccctgcccacacctttgtaaatagtcccttcattaaactctcctcaattacccccgtttgagtgtgccatctgtttcctgccgggaccctgactgataca

SEQ ID NO: 43 - DNA, 465 bp, Homo sapiens
  source 1..465: mol_type = genomic DNA
tgttgggagacaatcctccatgggcccctngcgctcctgcacgtcttgctgggtatgccaagaatgcaaggccctgaccgctctttacctcgggccatttctcagggttgtgtttgcagcgagcaaccttgagggatgaggtaatgtctccctccgggacaaagagcaggcttgcttactgcttgctataaaagcggtggattccccaagctcagtgttcctcngctgtaacgcaaacccactgcgtgcgcagcatccatctgggccctccgcgtcgcccccgtgggacttggggggncanggggaaccgacgcaaacatgctgatgctcatgctgcttgctgtgccgtgagtaataaagtcctttgtctctgacccaggagtctcgtgtcttctgccagcatccatgaaacagtaacaggctaacttattagcttgtaagtagggtaaaatcccagacctgaca

SEQ ID NO: 44 - DNA, 588 bp, Homo sapiens
  source 1..588: mol_type = genomic DNA
ggtgtgcttctctcacttggcaggctcctgcagccaggcacggcttctccagcgcccggctcctgcagcgcgcgcagcttctccagcgcccggctcctgcagcatggcacttctcagcatgcagctccgcagcagcagtgccagcagcttcccccggcaccctcctcgggcggttttgtagcggagtgcctccggtgagacacctccccgtgaacagctttccccggcaccctagagggcggatttccggcaagttccagagggcggatttccagcaagttccgccggcgcggcaccacagcgacttctctgccatccagtgagccacggccgtgccctctccaacaaggtctggatctcagccctggggggagggggctcttccttgggcgctctatctcagccctaggggtagtggctgctccttatatctgctattcctatattctttagagttctctttacttcttactagccaatccctcgttactccaatcccctgttatagttaataattctttatattaaactttccctgttcaaattactgtgtggtttctntctcctgattggacccngactgataca

SEQ ID NO: 45 - DNA, 758 bp, Homo sapiens
  source 1..758: mol_type = genomic DNA
tgtgccagttatcaatttattgcctctcagctccaaattcacccttcaatacctgctctgcgataatggactggactctttaagcatttctcctttacagtgagcacgatgttaagctttntcagtagagggcgctggagggacattgcaggaggaagggggcttctcttcctggttccggtgtgctgcgttttgctttttcttgctcctgctgcacggtcggtcagcggtgcgggtgtgtggggacatccggtggtgctctgccccagccgcgcgcccagagcgcgcagtccctcggcgacctcgcagccccggcctgggcccggtgaccaccttcccgcggccctcccgacgcggacaccgcgtgctccaggcctcgcgcccgcagcggcgccccgactccctctgcgcgcccgcccaccagcctcggctcgcctgcaccccggagggttgcttcctgcttgcccagcgactgcggaccagctctggcccgggcaanccagcgaacttctctgccatccagtgggctgcaaccacaccttctccaacgaggtctgaaccccagccttggggagggggccccccttccaagtttgtccttccttgggtactctccctcagccctagggtaccctttagagttctctttacatcttatagttactctcctatcatagcttaataattctttatattaaacttcccctgtttaaattactgtgtggtttctgtctcctgattggacccagactgataca

SEQ ID NO: 46 - DNA, 519 bp, Homo sapiens
  source 1..519: mol_type = genomic DNA
tgataacctacaggtcacatttggcaggcttccaaattaacccgcctnggggaggtcttgtgattcatggcnacatcctgtccctgagtaaagaatcttgtgagtccctcaaatcttatcgtgagttcctcaaactgttgacgtactgattaatacgtaacctactgacactgaaaaggacgctgatttgtttctgaatcatgaagttttgctgatttntttctgaatcatgaagttttactgattgtcttgcacgtagacattttagcctgtatgttgcaatctgtagccaatgattgtaacctctgtattgtaccctccaatgaaaaaggacaactccgatatgaggagtccccctcccttctcctaaactttcttataaaagccttccaacttgtaacagactctggaacacgcccaactttgttggtgtgtcttcccgggtcgatcctcacatttggcttccaataaacctttatcaaattatttctgcctcaacagccttaatttcggtcgaca

SEQ ID NO: 47 - DNA, 434 bp, Homo sapiens
  source 1..434: mol_type = genomic DNA
tgtggggacaagagtgactttattttaaatgctaatccgccatgtaacttctgactaaccccgagtccgggaatgcctccaaaatgtctagttgatgtattactctttatgtaggaacacctattcactgtaagtttcctccaaaacaacccttgatgctgttgcagaaatcataggctgtgacgcccgtagccacctacacattccttccagagcacgtatactttttccccaagatataagccctgggtctggggggttgcggtgcggagatctacctgtcttgcggccgcccaagaccacgcttctgtctgtaagttcccctaataaatcacccnanaccgacaaactggatttgtctgcctccttctttggtttctcggctccttcggcatttgggggtcgctttgcatatacggccctttcacggaaca

SEQ ID NO: 48 - DNA, 795 bp, Homo sapiens
  source 1..795: mol_type = genomic DNA
tgtgccggttattaagctattgtctctcagctccaaacccacccttctatactctgctttgtgatgctggggctgggactcgnnnntgcaaaccacatttctgctttgccagctggctctccctgttaggctctgccaatagggggcgctagagggagactgcaaggggctggaggggaggaagaagggacttgctccttcctgtctgctnctgtttcctgtctgcttcctgttcctgtcagcgtcaccccagcaacgcttcttcaccctggcagcagtngcttccagttnnttccagcagcagcagttgantccagtttgcgnngttccagtttccancantttttcccaacactcncagaaccagcctcatcgcgccccctcnnnnncctcagagacaccagcaccagccgggcagcgccccctcctcagaggtctgggtcccagctccgcggggcccctcctccgagctcagagacaccagcaccagccgggcagcgccccctcctcagaggtctgagntcccagctccgcggggcccctcctccaagcttctaagttttaataattccaacctcttcccttttgttcccccagccctaggggncgntggtagctgcttcctgcagttgctacctccgtgataccttagtgttctctttttgccttttcagttctctaatnnacaactttatacctagttaacccgnnttaacaattctttatattaaattctctctgttaaaataactggtgtggtttctgtcttctcctgactggaccctgactgataca

SEQ ID NO: 49 - DNA, 1304 bp, Homo sapiens
  source 1..1304: mol_type = genomic DNA
tgtaacaggatgttaaantggaagtttcggctgaggcaccaagatacaatagataccanattccaaagtgaggtgccagaccacnatgcagantttcaaaggaggtggccgaaaaccggttnnacgactcttaaacccctcacccacatcgagggtataaaagggtcgggangttggagatgaggggagatttgtgaagaggatttctttggagagagctgttggtgtgctgtgagtcccaccccacccccaaggagagaggagggggggtgctcccccttcacctcaagccnagngagaggggcttcaacgggaccactcggagagcttgatatgtgtcccctgcagtttgggggacacagtggactggtgtctgactcncgcctgggaaatctaaagggcgagagacgggctggccagctgctctgacggcggagcaaaggagaggtggctgcgctgggatcggcctgcactcccagagtttgtcggggcaaaggatgcgtgagtgtttcccgtggaccagatgtgggccacgcgngagagagagccggcgtggggtcgtcttaggtcctgtccaagagggccgcctggaggggcgaagggacctcagcagagagaagctggaggtaccgccggattcccaggggctgaggaaggagtaagcgaccagccggaagagagatgcattcgcccgcgtcaagggaactgcaggtgagagatccccagcgatggggggggggatctccgaagaacccacgaaagcgcccacgagagaaagagtcagctttaaacacctgccaggcccagagagcgcgaagccagctcacagacagtaccagtcaagtaagaactttcctgctccccttncctctcctccctccccncgctccaaccctggaggggtcagaaaccgcggttagcaagtgggggaggaggagnagggaaagagaaaaaagaagccaccacacccccttccccggctgcaggcttccngcctgcagcaggcccgagctgggggaggggagaagctttaactttaaatcaagttcggagttttgattattacatgggactggacattttaattactgaattgagactgtgttttgtgacttaaagtgaccgtaggactttttattacctaagagtgaccagaaaagtcatgggacctgcccgagttttcatccaggggcaggggaagaactanccccactgaataaatttaaagggacagtgggagacaaaaataaagttgctttntgattacatcccatgagtcntgcttgttcaacgtaccggttaca

SEQ ID NO: 50 - DNA, 820 bp, Homo sapiens
  source 1..820: mol_type = genomic DNA
tgaaaccgcctttgcaaagattatgacagtgagagaaatctgacatggctgactccatcttgcttctagcctcacaggctggctgtcctcgctcattcctgggcgtaggccaagctaaccntgggaggaatttagtttatagtttaactttgaagcaaggatgataatagtccctccctaaaactaaccccctccttgctcggggaccgaaaccgcctttgtaagactaatgaaaggccacaagattaggattatgggaggggcctgaattctgctaagatgtaggcatagttaaacgataaccagccattgtncccgnnncgnntttctataatcccttactgctcaggagtcatgtggccagaggtcacaagatttgtgacttccccaattgctcctatagataacatcactattgtagaacctaagattggtcttttgagatgtttttcagacttttgcattctggcgaccgactgaccccacccggacccgtgactcatgactcaaccggtcctgtggccgnnnncgnnncccacccagaggcggactcagcgcacgaggacccgttttccacacccctatgatttncatccccaaccaatcagcagcacccattccctagccccctgcccaccaaattatccntgaaaaaccctagcctccgagccttcggggagactgatttgagtaataanctccagtcctcccgcntggccggcctcgcgttaattaaactctttctctactgcaataccgcggtctcagtgaattggttttgtctgtgcagcgggcaggaagaacccgtcgggcgattaca

SEQ ID NO: 51 - DNA, 976 bp, Homo sapiens
  source 1..976: mol_type = genomic DNA
tgttagaaatttgatcggggatttcctgtgcagtctggagctgagaanaaggagatcatgacttgggctttgcagcccaaatatctttccttccccatcccccaaaatacacccaatcaaataaggttaggatgatggttaccaggcagcttgcaaaagcaagggcattccaccctccgaatttctggtcagaganagtanatatgtaaaacagaatatgctccattgttaagcaaacaccctntttattgatagtgggtcaacaagagcataggcaggtttagctatacagacaactaacagaatttgagatgcagaatattaaagctaagaatccaagatcaatcattcaaaatacaagtagatacgcagagaaaatatcacagcataagtagcagtnctaaaacgcaaagggctagagtcctgcnaaggcctttcctacgcagaagngccaaagcggagattcaggatcaacgctgttaaaatgcaaaatataatcaggagcaagcnctancgaatcctgcaaaggcctttcttaattaagcanttctganatacatncaactcctggttatagaaggnnnnnnnnnnnnnnnnnnnnnnnnnnnnnnnnccaagggaaaagcggngagaaagcagagagaaagagnagccggaaaatnccagctccttaaaagctctaaacaaggggcggagactcgctcctccctggccaatccttgatggctccggagagggcgccctgattaatactttgttacgtcattaaaaggggtcagcttaaggtaaggtaatggagntcccatgacctattgaccttgttacgtcattagagaggccanctttggcccagaaaatggcggattccttccctgtgattcacacattacccagaagcctctggcttcctggaattcttaataaaaattccaaatctctcggcaataactttcaaacaccacaattttcgctaaca

SEQ ID NO: 52 - DNA, 4166 bp, Homo sapiens
  source 1..4166: mol_type = genomic DNA
acattttctggcgacgaggatgggatcctgttaaaaagaaagctggacaacagcaggcagtgtaaatccactctcaagtactactggtaagcaattgaatacggcctccaccctggaacttgagcagaccctgtgcttaaaatggctgctttggagcatattgaagagtttgatgtcagtcancctgcttcctgggattcatatgctgagaggctttcattctatcttgaagcaaacaaaatagagggccctgaacagaagcatgctgtgctcctgacngtctgtggggcccaaacatntgccataataaggtctctcaccgtccctgcttcacctaagactaggacatttgatgaacttgtgggattattgaaggcgcacttctcaccagcaccatcagtcattgttcagagatttaaatttcacaaatggaatcaacagcctggtgagggcattgctgcttatattgcagagctgcgccatctgtctcaacactgcaattttggggatttcttagatgacatgttgcgggatcgccttgtatgcggagttcacgatgaagccctccaacggcgcctcttgactgaaccaaatttgacatttataatggcccaagaaaaggccctagccagtgagactgcatcactacatgtcacagaaattaaaggagcagcttctaatataaatgctgtccataacacgcaggcagagagctcttgccaacagggtcagcagaacacaggaaatgcncgttccagaatatctgcagatcagttcagacataccaacccctgctcaggctgcggagggttacaccaacgctctgcttgcagattcaaggatgcggagtgcagattctgcaagaggaagggccaccttgagagagtatgccgttcaaaatccattagaatgaagtccaggaactgtcgtagcagttcacatcacctggcatcaggtgatcttgattattcacatgacagtgaagcccaggaattgtatcatattcagagggtccagggcaagatgcctcaggatactgcagtatttgcaacagtactcataaatggccatgagtgcaagatggaagtcgactccggttgtggtatatcaataatcggtaaagataccttccacaatattttctcacaaggccctttgccaaagttgttgaggccaccatgcattcttcaagattacaatgatcacacagttgatgttgctggaagctgtgttgtgaatgtgcagcgtggctccttcacggagaagcttccacttatcatcgcaagggggcagcgcaaaagcctcttgggaaggaactggttcacaccactaggaatcagtgtccaaggcattcataccattagaacagcccgactacaggaagttgtcaacagatttcccaaggtattctccgaggaactgggagcttacaagagggagccagtgtcattccatctggatccagccattgcaccaattcacttgaaagctagaaatgtaccttttgctctaaggaaaaaaantgaagaagagcttgagcatctaatcaaacaaggtgccctcgaaccagttacacatacccggtgggctataccaattgcgccggtactaaagcctaatggtaaggtgagactttgtggagactacaagtgcactgtgaataaggcacttaagcagcacccatatccagtgcctgcagtcaatcaactccttgctgttcttgctggggaaaaggtctttgcgaaacttgaccttgcccaagcttatcaacagctgatcgtcagtgatgctactgcagacgcccaaacaatcatcactcatcgaggggcattccgagttaagcgccttcagttcggaatttctgttgcccctggaattttccagtgcttcatggagaccctgctttctaggattccaggtgtggtaccatatttcgatgatgtcctgatcatgggatcaacagaggatgaactcgcggaaagacttcaggaagtcctacatcgtttcgatacatcaggtatccgagtgaaaaaggagaaatgtgaaatcggaacttccagtgtcaccttcttaggctaccgcatcaatgctgctggtattcaccccactcaagataaggtacgtgcaatccatgacgcgcctactccaaaaacaaagcaagagcttcaagccttcctggggctactcaatttctaccatgtttttcttccacaaaaggcaaccattgctgagccacttcatcgcctactgggaaaaggcgttccgtggcgttggactcgccagcatgaggacgctttccggaatgtcaagcagttattgacttccgagtctgtgctagtacattacgatgaacataaaccactgtttcttaccactgacgcttcaccttacggtgctggagctgtgcttagccacaaaatgccagatggcactgaggcaccaattgcttactactcaagaacaatgtcatctacagaatggaactacgcacaaattgacaaagaagcccttgctgttatcgcgggagtcaagaagttccacaactacctctacggccgtgcctttacaatttgtactgatcacaagcctctttggggactcttttctaaggataagcctacgcccttaattatgtcaccacgtatgcagcgctggagtcttttgctcagtgcttatgactacgcgcttgtctacaagcctggaagagcaatatcgaatgctgatgccctgagccgtctgccactgcagatcccagattatgttgtgccacttccactggaagttttgatgttagaagacctccctgacccacctcttcaggctgatcaaatcgcacgattaactgcgtgggatcccagattggtccgtgtcttaaactgggtgtggagggggtgtccagtagagaaactgcctgaggaattcaagccattcacaagccatcaacatgagttatctgcccataaaggatgccttttatggggaaatcgcattgttattccagaaggcggtcatcacgttatcttggccacactacatgcagcacatccgggaattgttcgcatgaaagcattggctagaagctatgtctggtggcctggaattgattctgatattgaaaagactgtaaaggagtgtagcacatgccaggctactcggcataacccgccaaaggcacctacttttccatggaaagtgaccaagaagccttggtcaagaatccatattgattttgcaggaccattccaggggaagaacttcttgattgtggtagactccttctcaaaatggctcgaagtgctcctagttccatctcaaacctctgctgcaacaatcacagctctgcgccagttatttgcaacacacggattgccagacactatcgtttctgacaatgggtctgcatttacctcagttgagtttaaggaatttgtggacaggaaccttatccgagctgtcaccatcgcgcctcatcaccctcaagcaaatggtcaggctgaaaggatggtgcagactactaaggatgccctgaagcggattgttggaggagattggccaacaagacttgccaggtttctcatcgctcaacatgtcactccatgttcaacaacaggagtcagtcctgctgaattactaatggggcgacgtctcaagacttgcctcgaccgtctccatccagacttagatgaggacttacaagacaaacaagaatgtattctggatgctgctctgtctgcacctactcttcgctcatttttaccacaagagttagtctttgctaggaactatggagctggaccaaggtgggttcctgccactataattgaggtcacggggcctgtgtcatacaaggtccagagtgcagatgggccggtgtggcatcgccgtgttgatcaactgcgaaggcgttcctcccagggtgacttgacaacagcagaaccattagaagcatctcctctagagccatctggcgttgatttttcagctatacctcctgaggactcaccagagccacaaagtgaaggaagcaataattcttctctggccaccgcaactgctgaagtggagtcacctggtatttctacacccactagtataacacagagacagcctcgcaaaagggtacccccttgctggttaaaagattatgttagctaagggggaagggg

SEQ ID NO: 53 - DNA, 849 bp, Homo sapiens
  source 1..849: mol_type = genomic DNA
tgttgtcagtacttgggggctacagtgaggcgtcccccaagaacttggaaataagggaaataagattgccctctgagagttaaactataaaatcaacacataatggatcagagcagagatatgcaaagtcaccttaaagagacagaatggctcccaggccagcaagacttgactgtaacctgattgaatgtattaacatatctaaagaaagaatgtgtcaatcagaagggaggttggtcagccatggaagcatagaaaaaggagagtcacagaggcaaacgttggcagtgcccatccaagcactgtccaggggagagggagccttggccaacggacttcgagtattccggtcccattggcaaggccatccctactgtggagatggatgcatcaggctatcttagcctgatgccattaaacattcgaatcatntaactaactccccttgttctgattccatgctcaccaagcaaccttctgattataatttggcttcccatgtaggtctgcctacagggagagaatgcgctggtcatgcngacncgagtattctggtccncgtgacagttatatttctcattgtaaattaataaattggcattctggttttatacatcagtctcgtgagcttggttaatgtgagtccgcccctgagagatggatccgctctcggtgtgctgacaaaatcaggtttctgagcctgcaaggctgagggccagaccctggaccgtggtaaattcaccaaggtaatgccaccctgtgaggcctcttgggagacagccagccggcctttcccctggtgacactccantcnccacgatgttgccattggcattttaaattcttaca

SEQ ID NO: 54 - DNA, 938 bp, Homo sapiens
  source 1..938: mol_type = genomic DNA
tgtgatattgtgatataataagaaatatatatttggtctctgcccccggttcctggcacagagctcctaaaacccttgtaatttcctgagcgataggggtgctaggagcatcttttgttctaatatttggtctttgnccccggttcctgacacagagctcctaaaacccttggaatttcctgggtgataggagcgtcttttgttctaatgaggtgactcttggctgggggctcctggatagcctcaggatgggggctggtcgccagaggaaccaaccatgtgattagagggttggaactttcagccccacccccgacctccggggaggggagaggggctggagattgagttaatcaccaatggccaatgatttaatcaatcatgcctacgtaatgaagcctccataaaaaccctaaacgacggggttcggagagcttccgggttggtgaacacatngaggtgccgggagggtggcgcgcccggagagggcacggaagctccgcgccccttcccacataccttgccctatgcatctcttccatctggctgttcatctgtatcctttgtaatatcctttataataaaccggtaaacgtaagtaaagtgtttccctgagttctgtgagccgttctagcaaattatcgaacccgaggagggggtcgtgggaacccccgatttatagccggtcggtcagaagtacgggtnacaacctgggacttgcgactggcgtctgaagtgggggcagtcttgtgggactgagcccttaacctgtggggtctgacgctaactccaggtagntagtgtcagaattgaattgaattgtaggacacccagttggtgtccgctggagaattgnttggtgtgtgggaaaaaccccacacatttggtgtcagaagtgttgtgagagtagagaaaaacagtttgttttttcctacaca

SEQ ID NO: 55 - DNA, 707 bp, Homo sapiens
  source 1..707: mol_type = genomic DNA
tgttggggctcagaanacgataccccaaagtatggcgccttggcatgctgagtactttgaactgaaggagattggaaggcctcagaagcagcctcagaagcaaagtctctctctgaccttctcccgccctcctgtctcccgcccccgttctctcctccccgaagcgagtcatagaaaccagaattcctcttccccaaggcgggtcatagaaactagaacccctctcccccaaagcaagccataaaacctagaaangtcactctgaccttcccccgcctttctgtntnggagcnggccataaagaaattctctgacctnccttgtctganagtaggtcataagaccctcattccagaaggggtcctgccctatacccgggaggaaggaatgctacacagagaggccaagaagaatctgaacagacaggccttgctgggtttccccnctcagtttattaccattagatcataccccttttgtccaatcacatttctncacgactgtccattcttcatcgaacctaagcataaaaatanagttttccctgngtctttgggtcttcatttctgaaggctcccgtgtcacgtaaaactttgattaaataaatttgttatgcttttctcttgttaatctgtcttttgttataggggtgtcggccgtganccttgcgatgggtaggaaaggtatcacacctttctncccctaca

SEQ ID NO: 56 - DNA, 634 bp, Homo sapiens
  source 1..634: mol_type = genomic DNA
tgtcagaggcgtttgaaccagagcgactccatcttgaataggggctgggtaaaataaggctgagacctactgggctgcattcccaggaggttaggcattctnagtcacaggatgagataggaggtcggcacaagatacaggtcacaaagaccttgctgataaaacaggntgcggtaaagaagccggccaaaacccaccaaaaccaagatggcgacgaaagtgacctctggtcgtcctcactgctcattatacgctaattataatgcattagcatgctaaaagacactcccaccagcgccatgacagtttacaaatgccatggcaacgtcaggaagttaccctatatggtctaaaaaggggaggaaccctcagttccgggaattgcccacccctttcccggaaaactcatgaataatccaccccttgtttagcatataatcaagaaataaccataagnatagncagccagcagcccacgccgctgctctgcctatggagtagccattcttttattcctttactttcttaataaacttgctttcactttactctatggactcgccccgaattctttcttgcgcgagatccaagaaccctctcttggggtctggatcgggacccctttccggtaaca

SEQ ID NO: 57 - DNA, 487 bp, Homo sapiens
  source 1..487: mol_type = genomic DNA
tgagaccctgcttgngacacatgtgaaaaatgcaggggaaaaccagtcccctgtggagtgtgaaaataattaagtggcaggcaattagactgaggtggctctagtgccctgggttcctacttaaaaaaaaatctaactcaaatgcattttttgtaaattactacnttaggggaaaacaaaattcaggcttaaccaaccataaaccgccaattaanctctgattacataaccaggaaatttccacctggatngtacaaatnaagaaactacgtaactgtacctaaccaattattgaatttggtttgcttcntcatgcaccttataaaagcctttccttcaagcccctcccgtggaccacaaactacaaaccatagctgggtgctctacgattcntgaatcactctttgattaaattctttaatatttttgcggtgactcccataaatttttaacaggagaaaggagggactggggaccccacggacca

SEQ ID NO: 58 - DNA, 555 bp, Homo sapiens
  source 1..555: mol_type = genomic DNA
tgaggtaggagaccggcaggacttgttttctggtcacaaccctgctgaccaaaacaggatctggtccagacaggataaagtgaagaaaccggcaggaaccagcagatggcgacgaaagcgatccctagctgccctcattgctcattagcataagacactcccaccagcgccatgacagtttacaaatgccatggcaacgacccggaagttaccacccctttccatggcaacgacccggaagttaccgcccctttcctagaaagttctaaataacccgcccctcaatttgcattgacccgccccttaatttgcatgtaattgaaagtgggtntaagtgagtataaatacagttgccaagagcccatacgttgccgactctgggcgcactgcctatgagttagccctgctccgcaaggagcagtaccgttcaataaaagattgctgtctaacaccaccggctcgcccttgaattctttcctgggcgaagccaagaaccctcccgggctaagccccaattttggggctcgcctgtcctgcatcatctggctaccatca

SEQ ID NO: 59 - DNA, 484 bp, Homo sapiens
  source 1..484: mol_type = genomic DNA
tgaggtaggagatcagcaggacttgttttccgagcaccggtcacgaccctgctgatcaaaacaggatgtagcaaagaaaccggccaaaaccagctaggaccaggaattataatgcattngcataagacactcccaccagcgccatgacagtttacaaatgccatggcaacgacccggaagttaccttatatggttccgggaactccccgccccttttccagaaagttcgtgaataacccgccccttatttagcatataattaagagtaggtataaatatagctagccagcaatccacgagtgctactctgggccgctctgcctatggggtagccctgctctgtctatggagcagccattttgctgtacactgttgctctaataaacttgctttctttcactgtcggctcgctcttgaattctttcctgngcgaagccaagaaccctcccgggctgagccccaattttggggttcgcctgcatca

SEQ ID NO: 60 - DNA, 563 bp, Homo sapiens
  source 1..563: mol_type = genomic DNA
tgtgcagaaaagagttaacatagcaggcctgagactgctatccttagaaaggcctgcttgcaaggttggcccttggctggcgtctgggaacttggatttcgggagggttcccaccattcccgnaactgataagagtggctcactgtgcctaaactgtttgtacaaacaatgtggtttatgctgaacacctgctttccttctgggagtctggaattttggtacgtgctaggcagagggtgcctacgtgaccagcccccaataaaaaccctgggcactgagtctctaatgagcttccctggtagacaacatttcacacgtgttgtcacaactcgttgctggaggaattaagcgcgtcctgtgtgactccactgggagaggactcttggaagcttgcgcctggtttcctccggacttcgccccatgcgccttttccctttgctgattttgctttgtatcctttcgctgtaataaatcatagccgtgagtacgactatatgctgagtcctgtgagtcctcctagcgaatcaccgaacctgggggtggtcttggggacccccgacaca

SEQ ID NO: 61 - DNA, 700 bp, Homo sapiens
  source 1..700: mol_type = genomic DNA
tgtgtagtaaagaatttaaccttgcccaaagagaggtctggcctttgccctcggctcctgggaggtaatctctaagcccttggaatgtcntgcctgataggagtgtctttgtttgcctgggggccttgggccacgccggatagtctaacaatgtgatttagggtgggggctggccacgccagaaagaccaacnatgtgatttagggtgggggctttgggtcacgcggtatcagcccgactccggagggnctggagactgagntcagccacgtgggcaatcaatcaatcatgcctacgtgatggagccccaataaaaactctggacaccgaggctcgggtgagcttccctggttggcaatactccgtgcgtattgtcacacatcgatgccgggagagtaacgctgtccctgactccacggggagaggacaactggaagctccgcgtttggaaccctcccggactctgccctatgtaccgcgcgcctcttcccttggctgattttaatctgtatcctttccctgtaataaaccgtaaccgtgagtataacagctttcagtgagttctncgtgagtccttctagcgaattatcgaacctgagggtggttttgggaaccccccgaacttgcagttggtgtcagaagtgagggcggtcttgtgtggactgtgtccctctaactttgcagttggctaactttcaca

SEQ ID NO: 62 - DNA, 1735 bp, Homo sapiens
  source 1..1735: mol_type = genomic DNA
gaaattggtaccgagagtgggggtgctgctataacaaatacctaaaaatgtggaagcggctttggaactgggtaatgggtagaggctggaagagtttngaggtgcatgctagaaaaagcctanattgccgtgaacggancgttaagggcgattctggtgagggctcagaagaagaggagagctgtagagaaagcctcaatcttcttagagattacctaagtggtcgtgancagaatgttggtagaaatatggacggtaaaggccattctgatgaggtctcagacggaaatgaggaacatgttattggaaactggaggaaaggccatccttgttataaagtggcaaagaacttggctgaattgtgtccgtgtcctagngctttgtggaaggcagaacttangagcgatgaantaggatatttggcggaagaaatntctaagcaaagtgttgagggtgcngcgtggcttctcttgactgcttatagtaaaatgcgagaagagagaaatganttaaagacggaatttataatcaaaagggaagcagaacntaaagatttggaaaattctcagcctggccatgttgnaaagaatgaaaaagcgtgttcgggagagaanaccaagggtgtggccaagcgaccgtttgataaggagattagtatggatagaaggaagccaggtgctattcatcaagacaatggaagaatgaccccgaaggcatttcggagatcttcgaggctgcccctcccatcacaggcccagagtgcnagggcctngagggcagaacggtttcaagggcaggnnccantccccactgcccagngccgcctcagtctgctccccgtcttcggccgcccgnncagctgtggctcaagcgggcccaggtgcggctcgggccgccgctccggaaggcacaagccgtaaaccttggcggcgtccacgtggtgctaactctgcaggcgcgcagagtgcangagctgtgggggcatggctncctccacctagatttcaaagatgcgagacctggggcccaggcagaganctcgcggggcagggccaccgcagagagcccccactagggcaatgcccagtggagccgtggggtcgggnctgcagagagcccccactagggcaatgcctagtggagccgtgggggcggggccgcccccgagaccccagaactgtagagccaccagcgtgcaacnccagcctgggagagccgcaggcacgngactccaacccgtgagagctgccgcgtgggctgcgcccagcaaagccatgggggcggggctgcccggggccttgggggcccaacccccgccccagtgtgtccggaaggcgggacatggagtcaaagaagattattctcgagccttaagatttaatgttgtttgccctgttgggttttggacttacttgggacctgttacccctttcttctttcctatttctcccttttggaatgggaatgtctatcctatgcctgtcccaccattgtattttggaagcacataacttgtttgatttcacaggctcacagctggagaggaattttgcctcaggatgaatcgtaccttgagtctcacccatatctgatttagatgatatttagatgagactttggactttagactttngagttgatgctggaacgagttaagactttnggggctattgggatggaatgaatgtattttgcatgtgagaaggacatgaattttggggggccaggggcggaa

SEQ ID NO: 63 - DNA, 374 bp, Homo sapiens
  source 1..374: mol_type = genomic DNA
tgctatggactgaatgtttgtgtccccccaaaattcatatgttgaagccctaacccccaatgtgatggtatttggaggtggggcctttgggaggtaattaggnttagatgaggtcatgagggtggggccctcatgatgggattagtgcccttataaaagagacaccagagagctctcttnccccttccgccatgtgaggacacagcgagaaggcgccgtctgcaagccaggaagagagccctcaccagaaaccgaatctgccggcaccttgatcttggacttcccagcctccagaactgtgagaaataaatttctgttgtttaagccacccagtctatggtattttgttatggcagcccgagcngactaagaca

SEQ ID NO: 64 - DNA, 365 bp, Homo sapiens
  source 1..365: mol_type = genomic DNA
tgctatggtctgaatgtttgtgtccccccaaaattcatatgttgaaacctaacccccaangtgatggtattaggaggtggggcctttgggaggtgattaggtcatgagggcggagccctcatgaatgggattagtgcccttataaaagaggccccagagagctccctcgccccttccgccatgtgaggacacagcgagaaggcgccgtctatgaaccaggaagcgggccctcaccagacaccgaatctgccggcgccttgatcttggacttcccagcctccagaactgtgagaaataaatttctgttgtttataagccacccagtctatggtattttgttatagcagcccgaacggactaagaca

SEQ ID NO: 65 - DNA, 408 bp, Homo sapiens
  source 1..408: mol_type = genomic DNA
tgctatggtttgaatgtttgtgtcccctccaaaattcatgtgttgaaacttaatccccaatgcgatagtattaagaggtggggcctttaggaggtgattaggtcatgagggctccgccctcatgaatgggattagtgcccttataaaaggggcttgagggagcgagttcggcccttcttgcccttccgctcttccgccatgtgaggacacagcgttcctcccctccggaggatgcagcaacaaggcgccatcttggaagcagagagcaggccctcaccagacaccgaacctgccggcgccttgatcttggacttcccagcctccagaactgtgagaaataaatttctgttctttataaattacccagtctcaggtattttgttatagcagcacaaacggactaagaca

SEQ ID NO: 66 - DNA, 390 bp, Homo sapiens
  source 1..390: mol_type = genomic DNA
tgttatgggctgaattgtgtccccccaaaattcatatgttgaagtcctaacccccagtacctcagaatgtgactgtatttggagatagggcctttaaagaggtaattaagttaaaatgaggtcattagggtgggccctaatccaatatgactggtgtccttataagaagaggagattaggacacagacacgcacagagggaagaccatgtgaggacacagggagaaggcggccatctgcaagccaaggagagaggcctcagaagaaaccaaccctgccgacaccttgatctcggacttccagcctccagaactgtgagaaaataaatttctgttgtttaagccacccagtctgtggtactttgttatggcagccctagcaaactaataca

SEQ ID NO: 67 - DNA, 467 bp, Homo sapiens
  source 1..467: mol_type = genomic DNA
tgttatgggttgaattgtgtccccccaaaattcatatgttgaagtcctaacccccagtacctcagaatgtgaccttatttggaaatagggtcnttgcagatgtaattagttaagatgaggtcatactggagtagggtgggcccctaatccaatatgactggtgtccttataaaaaggggaaatttggacacagacacgcacacagggagaacgccatgtgaagatgaaggcagagatcggggtgatgcntctacaagccaaggaacgccaaagattgccagcaaaccaccagaagctaggagagaggcatggaacagattctccctcacagccctcagaaggaaccaaccctgccgacaccttgatctcggacttctagcctccagaactgtgagacaataaatttctgttgtttaagccacccagtttgtggtactttgttacggcagccctagnaaactaataca

SEQ ID NO: 68 - DNA, 461 bp, Homo sapiens
  source 1..461: mol_type = genomic DNA
tgttgtgggttgaattgtgtcccccaaaaagatatgttcaagtcctaacccccggnacctgtgaatgtgaccttatttggaaatagggtctttgcagatgtaatcaagttaagatgaggtcatactggattagggtgggccctaatccaatgactggtgtccttataagaagagggaaatntggacacagagacacacagggagaaggccatgtgaagacggaggcagagattggagtgatgcggctacaagccaaggaacgccaaggattgccggcaaccaccagaagctaggaagaggcaaggaaggattctcccctagagccttcagagggagcacggccctgccgacaccttgatttcggacttctggcctccagaactgtgagagaataaatttctgttgttttaagccacccagtttgtggtantttgttacggcagccctaggaaactaataca

SEQ ID NO: 69 - DNA, 505 bp, Homo sapiens
  source 1..505: mol_type = genomic DNA
tgtggtaggcagaataatggccccccaaagatgtccacgtcctaatccccggaacctgtgaatatgttaccttacatggcaaaagggactttgcagatgtgattaagttaaggatcttgagatggggagattatcctggattatccgggtgggcccaatgtaatcacaagggtccttataagagggaggcaggagggtcagagtcagagaaggagatgtgacgacggaagcagaggtcggagtgacgacgttgctggctttgaagatggaggaaggggccacgagccaaggaatgcgggcggcctctagaagctggaaaaggcaaggaaacggattctcccctagagcctccagaaggaacgcagccctgccgacaccttgattttagcccagtgagacccatttcggacttctgacctccagaactgtaagataataaatttgtgttgttttaagccactaagtttgtggtaatttgttacagcagcaataggaaactaataca

SEQ ID NO: 70 - DNA, 653 bp, Homo sapiens
  source 1..653: mol_type = genomic DNA
tgtggtaggcagaattctaagatggcccccaagattcccgccccctggtgtacacgccctgtataatcccctccccttgagtgtgggcgggacctgtgaatatgatgggatntcactcccgtgattaggttacgttatatggcaaaggtgaagggattttgcagatgtaattaaggtccctaatcagttgactttgagttaatcaaaagggagattatcctgggtgggcctgacctaatcaggtgagccctttaaaagagggtctagaggtcagagacggaagaagtcagagagattcgaagcagcagagacgctctcctgctggccttgaagaagcaaactgccatgttgtggagagggccacgtggcagggaacggcgggcggcctctaggagctgagggcctcagtcctacaaccgcaaggaactgaattctgccaacaaccagtgagcttggaagaggaccccgagcctcagatgagaccgcagccccggccgacaccttgatttcagcctngtgagaccctgagcagaggacccagctaagccgtgcccggactcctgacccacggaaactgtgagataataaatgtgtgttgttttaagccgctaagtttgtggtaatttgttacgcagcaatagaaaactaataca

SEQ ID NO: 71 - DNA, 654 bp, Homo sapiens
  source 1..654: mol_type = genomic DNA
tgtggtaggcagaattctaagatggcccccaagattcccaccccctggtgtacatgccctgtataatcccctccccttgagtgtgggcgggacctgtgaatatgatgggatatcactcctgtgattaggttacgttatatggcaaaggtgaagggattttgcagatgtaattaaggtccctaatcagttgactttgagttaatcaaaagggagattatcctgggtgggcctgacctaatcaggtgagccctttaaaagagggtctggaggtctttctgaagaagtcagagagattcgaagcagcagagatgctctcctgctggccttgaagaagcaagctgccatgttgtggagagggccacgtggcagggaacngcgggcggcctctaggagctgagggcctcagtcctacagccgcaaggaantgaattctgccaacaacctgagtgagcttggaagnggaccctgagcctccagatgagaacgcagccccggccgacaccttgattncagccttgtgagaccctgagcagaggacccagctaagccgtgcccggactcctgacccacggaaactgtgagataataaatgtgtgttgttttaagccgctaagtttgtggtaatttgttacgcagcaatagaaaactaataca

SEQ ID NO: 72 - DNA, 680 bp, Homo sapiens
  source 1..680: mol_type = genomic DNA
tgtggtaggcagaattctaagatggcccccaagattcccgccccctggtgtacacgccctgtataatcccctccccttgagtgtgggcgggacctgtgaatatgatgggatatcactcccgtgattaggttacattatatggcaaaggtgaagggattttgcagatgtaattaaggtccctaatcagttgactttgagttaatcaaaagggagattatcctgggtgggcctgacctaatcaggtgagcccttaaaagaggcatgggccctccagagagaagaacagagagattctcctgctggccttgaagaagcaagctgccatgttgtgagagggcctatggagagggccacgtggcaagganctgngggcggcctctaggagctgagagcggcccccggccgacagccagcaagaaaacggggacctcagtcctacagccgcaaggaantgaattctgccaacaacctgaatgagcttggaagnggaccctaagcctcagatgagaacgcagccctagccgacaccttgattncagccttgtgagaccctgagcagaggacccagctaagccgtgcccggactcctgacccacagaaactgtgagataataaatgtgtgttgttttaagccgctaagtttgtggtaatttgttacgcagcaatagaaaactaataca

SEQ ID NO: 73 - DNA, 627 bp, Homo sapiens
  source 1..627: mol_type = genomic DNA
tgtggtaggcagaatggccccccaaagatgtccacgccctaatccccggaacctgtgaatatgttacgttacatggcaaaagggactttgcagatgtaattaaggttacggaccttaaaatagggagattatcctggattatccgggtgggcccaatctaatcacatgagcccttaaaagcagagaactttctccggctggagtcagagagatgcggcagaaggggaagtcagagagattcgaagcgtgagaaggactcgacgcgccgttgctggctttgaagatggagggggccacgtgncaaggaatgcgggcggcctctaggagctgagagcggcccccggctgacagccagcaaggaaacggggacctcagtcctacaaccgcaaggaactgaattctgccaacaacctgaatgagcttggaagcggattcttccccagagcctccaganaagagcncagcccngccgacaccttgatttcggccttgtgagaccctgagcagagaanccagccgagcccacccggacttctgacctacagaactgtgagataataaatntgtgttgttttaagccgctaagtttgtggtaatttgttacggcagcaatagaaaactaataca

SEQ ID NO: 74 - DNA, 624 bp, Homo sapiens
  source 1..624: mol_type = genomic DNA
tgtggtaggcagaattctaagatggcccccaagattcccggcccctggtgtacacacaccttctcccagttattcaatcaaacactaatctaggtgctgctgtgaagggattttgcagatgtaattaaggtcccaaatcagttgaccttaagatagggagattatcctgggtgggcctgacctaatcacgtgagcccttaaaagggactgggctcttcctggagaaagagattcgaagcgtgagagggattcgacgcgagggagattctccgttgctggctttgaagatggagggggccacgtggcaaggaatgcgggcggcctctaggagctgagagcggcccccggctgacagccagcaaggaaacggggacctcagtcctacaaccgcaaggaactgaattctgccaacaacctgaatgagcttggaagcggaccctgagctccagatgagaacncagcccggccgacaccttgatttcagccttgtgagaccctgagcagagaacccagccacgccgtgcccggacttctgacctacagaactgtgagctaataaatgggtgttgttttaagccgctaagtttgtggtaatttgttacgcagcaatagaaaactaataca

SEQ ID NO: 75 - DNA, 548 bp, Homo sapiens
  source 1..548: mol_type = genomic DNA
tgtggtagccagcctccaagatggcccccaatgatccccgcctcctggtattcacgcccttgtgtagtcccctcccacactgaatagggctggcctgtgtgaccaataggatatngcggaagtgacggtgtgtgacttccgaggctaggtcataaaagacattgcggcttccgccttgctctctcttggatcactcgctctgggggaagccagctgccatgtcgtgaggacactcaagcagccctgtggagaggcccacgtggcgaggaactgaggcctcctgccaacagccagcaccaacttgccagccatgtgagtgagccatcttggaagcggatcctccagccccagtcaagccttcagatgactgcagccccggccgacatcttgactgcaacctcatgagagaccctgagccagaaccacccagctaagccgctcccgaattcctgacccacagaaactgtgtgagataataaatgtttattgttgttttaagccgctaagttttggggtaatttgttacgcagcaatagataactaataca

SEQ ID NO: 76 - DNA, 1489 bp, Homo sapiens
  source 1..1489: mol_type = genomic DNA
gattttggtacctggaagtggggtgctgccgtaacaaatacctaaaaatgtgggagtggctttggaaccgggcagtgggcggaagctggaaggattttgaggagcgtgntagngaaagcctaaantgccttgaacagactgttagtagaaatctggactttgaggangctgccggtgagggctcaaagggaagtgaggaanatgttattggaaactggaggaaaggggatccttgttatgtagtggcagaaagcttagcaanactgtcgcctgcagttatgtggaaagtagaaaatgtanctaatgaactnggtgatctagctaaggagatttccaagcaaagtgttgaaggtgccgcctggtttcttcttgctgcttatagtaaaatgcgagaggagagagataaactgagggaagaactgttaaacaaaaaggagccaggacttgatggttttgaaaattctcagcctctccagatggcaaangatgctaaaattaagaaatggcttccgagcaaagatcaaatccagggcactgtcaggaaaacatggtctaaagatgaagccgagggtgtgactgtaaaatcctttgttaagacctcagaaagatcaaaggtggtgcctcagagtactattcagtcanacaaaaggccctttaaagagattaagggtgtgcctcacagatcctctcaatcaaacaatagggcttctaagaagcttaagggcgttgtccctcagccgtctcagcagaagcccaaggtagagaagggcttatctcgaagagatttgtgggtgtggcttttgtctaatggagtgaaccccagtaagattcacaggagacccacaaagtttttaagagaattatatcagcagaaacactgccagcttggactgaaagggacagagacagtacaaaatgaaaagaggcctttggacccccaaanttctacnggcaggaagcaggctganaaaactactcagctgcaaacacgtgctacctttcatgaaaaaggaaggatgactcagagggcggaaccaagagcccagagggcggagccaagagccacggagaattattcccaggccttgagacctaatcaaggaacttccaacatttgcccggctggatttcagaattgctatggaccagtgactcctttgtgcctcccattttccccctttttgaacgggaatgtctatagcggttatcctatgcctgtcccaccattgtatgttgggtgtgtngggggcagataacttgtctctttagtttcacaggtctncagatcgagaggaactgtactcgaggagctgtacttaaggaactacacccgaggagcctcatccacacctggacctgatttagatgatgagattctggactttgagctgatgctgtaatgggatgagacttttggggatcttgggaggggggtgagtgtattttgcatgtgggagggacgtgaatcattgggggccagagggcagac

SEQ ID NO: 77 - DNA, 569 bp, Homo sapiens
  source 1..569: mol_type = genomic DNA
tgtggtggttttaaaatatgtccacaaattctttgatactcctcccttcaagaggtggagcctaattcccctccccttgagtgtgggctggacttagtgactcgcttctaacgaatagaatatggcggaagtgacggtgtgtgacttccgagactaggtcataaaaggcattgcggcttcctccttgctctctctcttggatcactcgctctgggggaagccagctgccatgtcgtgaggacactcaagcagccctatggagaggcccacgtggcgaggaactgaggcctcctgccaacagccagcaaggaactgaggcctcctgccaacagccatgtgagtgagccatcttggaagcggatcctccagccccagtcaagccttcagatgactgcagccccggccgacatcttgactgcaacctcatgagagaccctgagccagaaccacccagctaagccgctcccgaattcctgacccacagaaactgtgtgagataataaatgtttgttgttttaagccgctaagttttggggtaatttgttacgcagcaatagataactaataca

SEQ ID NO: 78 - DNA, 560 bp, Homo sapiens
  source 1..560: mol_type = genomic DNA
tgtggtaggcagcctctaagatggcccccaatgatccccgcctcctggtattcacgcccttgtgtaatcccctccccttgagtgtgggctggacctagtgactcgcttctaacgaatagaatacggcaaaagtgatgggatgtcacttccgagattaggttacaaaaagactgtggcttccgtcttgcgcgccctctcttgctctctcgcttgctcgctctganggaagcnagctgccatgttgtgagctgccctatggagaggcccacgtggcaaggaactgagggcggcctccggccaacagccagcgaggaactgagncctgccaacaaccacgtgagtgagcttggaagcggatcctcccccagtcgagccttnagatgaccgcagccccggccgacaccttgattgcagcctcgtgagagaccctgagccagaggcacccagctaagccgcgcccggattcctgacccacagaaactgtgagataataaatgtntgttgttttaagccgctaagttttggggtaatttgttacgcagcaatagataactaataca

SEQ ID NO: 79 - DNA, 591 bp, Homo sapiens
  source 1..591: mol_type = genomic DNA
tgtggcggtttttaaaacatggccgcaaattctttgacactcctcccatcgagaggtggggtctatgtcccctccccttgaatctgggcgggcttgtgactgcttcgaccaatagagtacggcggaagtgacgctgtgtgacttccgaggctaggtcataaaaggccatgcagcttccgccttgttcgctggaacactcgctcttggagccctgagccgccatgtaagaagtccgactaccctgaggccgccatgctgtgaggaagcccaagccacatggagaggccacgtgtaggcgctccggtcgacagtcccagctgagcccagccttcgagtcatcccagcccaggcgccagacatgtgagtgaagaagcctccagatgattccagcccccagccgttcgagtcacccccagccgttcgagtcttcccagctgaggccccagacatcgtggagcagagacaagccatccccgctgtgccctgtccgaattcctgacccacagaatccgtgagcataataaaatggttgttgttttangccactaagttttggggtggtttgttacgcagcaatagataactggaaca

SEQ ID NO: 80 - DNA, 579 bp, Homo sapiens
  source 1..579: mol_type = genomic DNA
tgtggcagattgtattttccaaagatggccgcaacaatatctcccatcccacatgctcttcttacaatgtgacnttgacactcctcccatcgagaggtggggtctatgttccctccccttgaatctgggcgggcctttgtgactgcctcgaccaatagaatgcggcggaagtgacgctgtgtgacttccgaggctaggtcataaaagtgccatgcagcttccgccttgctctcttgggacgctcgctcttggaacccagccgccatgctgtgaggaagcccaagcagcccatggagaggcccacgtggagaggaactgaggcttccggccgacagccccagctgagntcccagccgacagccagcatcaacttgccagncatgtgagtgagccatcttgnaggtgattccagcccccagcctttgagccgccccagctgacgccgcgtggagcagagacgagccgtccccgccgagccctgcccaaattgcagattcgtgagcaaaataaatgattgttgttgttttaagccactaagttttggggtggtttgttacgcagcaatagataaccggaaca

SEQ ID NO: 81 - DNA, 595 bp, Homo sapiens
  source 1..595: mol_type = genomic DNA
tgtggcagattgtattttccaaagatggccgcaacaatatctcccatcccacatgctcttcttacaatgtgaccttgacactcctcccatcgagaggtggggtctatgtcccctccccttgaatctgggcgggcctttgtgactgcctcgaccaatagaatacggcggaagtgacgctgtgtgacttccgaggctaggtcataaaaaggccatgcagcttccgccttgctctcttgggacgctcgctcttggaacccagccgccatgctgtgaggaagcccaagccacatggagaggccacgtgtaggtgttccggccgacagccccagctgaggtcccagccgacagccagcatcaaccgccagacatgtgagtgaagaagcctccagatgattccagcccccagccgtcgagtcacccccagccttcgagtcttcccagctgaggccccagacatcgtggagcagagacaagccatccccgctgtgccctgtccgaattcctgacccacagaatccgtgagcataataaaatggttgttttangccactaagttttggggtggtttgttacgcagcaatagataactggaaca

SEQ ID NO: 82 - DNA, 549 bp, Homo sapiens
  source 1..549: mol_type = genomic DNA
tgtggtagattgattgcaaaaatggccncaattcttcacccctccctgtatccacgccctttgcaatgtgactttgcagctcctcccatcaagaggtggagtctatttccccaccccttgaatctgggctggccttgtgacttgctttggccaatagaatgcggcggaagtgacgntgtgccagttccgagcctaggcctcaagaggccttgcacgcttccgctctctctcttggaaccctgccaccgccatgtgaacaagcccgggctagcctgctggaggatgagagaccacgtggagcagagccgagccgtcccagctgaggccatcctagaccagccagcccccagccaagatcagcagagccgcctacccgacccgcagctgaccgcagacgcatgagtgagcccagccgagaccagaagaaccgcccagctgagcccagcccaaattgccgacccacagaatcgtgagctaaaataaatggttgttgttttaagccactaagttttggggtggtttgttacgcagcaatagctaactgataca

SEQ ID NO: 83 - DNA, 1409 bp, Homo sapiens
  source 1..1409: mol_type = genomic DNA
gaaattggtacctagaagtggggtgctgccgtaacaaaaacctaaaacatgtggcattggctttgggaccgggcggcgggcagaggctggaaaagcgncgaggagactgttagtggaggctggaagagcagtgaggaaactgctattggaggctggaaaaanggcgacccgtgttatgtagtggcggaacanttggcaaaactgtcgcctgcggtaacntggaagatagaaaatgtacctaatgaacttgtggatctggctaaggagatttccaggcagaatgttgaaagtgccaactggcttcttttagctgcgtatgataaggtacggnaagagagagatgagctaaagaaggaactgttcagtttncaagcagaatttagaggaaatatagagganccaggacttgctgggttggaaaataaaactgtttctcatctccagtctctccagccggcaaaagattctcaaagtaagaaatggcctcagggcaaagatcaaatcaagggtgtggctgtaagaccctttgttaagacctcagaaagatttaaggcggtgcctngtagaccctctcagctagacaaaagggcttctaagaatcttaagggcgttgtcccacagcagcctgacacgcagcccaaagtagagagaggcctgtctcgaaaagaattgtgggtgtggcttttgtcnaatggagtggactnnaatnngatncataggaaacccacaaagtttttaagagaattgtattggcaaaagcaccgccagcttggactaaaagggacagagacagttcaaaatgaaaagaggcctctgggcccccaactttctacgggcaggaagcaggctgagaaagctactcagctgcaaacacgggccatttcttatggaaaaggaaggacgnctcagagggcagagccaagagcccagagggcggagccaagagccanggagaacaatggactagggaaccactcccagggagcagaaccgggccctaatcaaggaacattccctgcccccggagtaggggaacctggcaacatgtgcccggctggatttcagaattgctatggaccagtgactgctatgtgcctcccattcttcccctttttgaatgggagtgtctattgcggttatcctgtccctgtctcaccattgtatgttgggtgtgtggggggcagataacttgtctttttagttcacaggtctccggatcaagaggagccacaccctgaggagctgcacccgaggagcctcatccgcatccggacctgatntagatnacgagatcctggacttcgagcctgatgccgtaattggatgagacttttgggggtcttgggangggggtgagcgtattttgcatgtgggaggaatgtgaataatttgtggccagagggcagact

SEQ ID NO: 84 - DNA, 555 bp, Homo sapiens
  source 1..555: mol_type = genomic DNA
tgtggtagattaaagatggccgcaaattctttgacactcctcccatcgagaggtggggtctatntcccctccccttgaatctgggctggccttagtgactgctttgaccaatagaatgcggcggaagtgacgctgtgcgacttccgaggctaggtcntaagaagccttgcagcttccgcctnggtctcttggaacgctcgctctgggagccctgagccgccatgtaagaagtccgactaccctgagaccgccatgctggagaggccacgtgnaggcgctctggtcgacagtcccagctgagcccagccttccagccatccccgccaaggcgccagacatgtgagtgaagccgtcttggaccctccagaccagcccanccgccagctgaataccaccgagtgacctcagtcgacgccacgtggagcagaagaaccgcccagctgagccctgcccgaattcctgacccacaaaatcgtgaganataataaaatggttgttgttttaagccactaagttttggggtagtttgttacgcagcaatagataaccggaaca

SEQ ID NO: 85 - DNA, 489 bp, Homo sapiens
  source 1..489: mol_type = genomic DNA
tgtggtagactgttacattggtggcccccaatgaaccacgcctcccggtattcacgcccttgtgtagtcccctcccacattgactctgggcttggccatgtgacttgctttggccaatgggacattagcaaacgtgatgcaagcagaggcttganaagcgcttgcgcattggggcttgtcctcttggaacgctccctcttggaanccagctgccatgctgtgaagaagcccaggctagnctgctggangatgagaggccacgtggagagaggccctggaggatgagaggccatcttggacgttccagccccagccgagctcccagctgaatgcagccacatgagtgaccccagctanaccacgtggagcagaagaaccgcccagctgagcccagccaacccacagaatcgtgagaaataataaatcgttgttgttttaagccactaagttttggggtggtttgttacgcagcaatagataactgaaaca

SEQ ID NO: 86 - DNA, 411 bp, Homo sapiens
  source 1..411: mol_type = genomic DNA
tgtggtagtcattagtgctgttcaccaaatatttccggctctctnccttccgggcacatggtaggattgcacttccccgcccccttgaagttaggcgtggccatgtgacttgctttggccaatgaaatgtgagcggaagtgacgtgtgtcacttccgggcggaagctttaagagccagtgcgtgattcgccacgttcccctttccctctgccacggcgaccggcaatgttccagatggtggctgctccgtcagcctgggtcccggagtgaggacgacgtggagcagagcccccagccgacccgcgatggacatgtagcatgagcgagaaataaacctttgttgttttaagccactgagatttgggggttgtttgttaccgcagcataacctagcctatcctgactgataca

SEQ ID NO: 87 - DNA, 512 bp, Homo sapiens
  source 1..512: mol_type = genomic DNA
tgtggcagagactgctagttgtcccccaatatccattctccccttcttccttagtaatagaacccccgatttttagctgggcacatggccgcccagaataaagactacatttcccagcctcccttgcagctaggtgtggccatgtgactaagttctggccaatgggatgtaagcggaagtgncgtgtgcaacttccgggaagtgtccttaaagggagggggcgtgcccttcttttccccttcctccttcctgctgnctggaatgcggacgtgatggctggagctcnagcagccatcttggaccatgaggtggaagccacgtgctgaggatggcggagcaacaagatagaaggagcctgggtccctgatgaccgtggagccgccataccagccctggactgcctacctccggacttcttttacgtgagagagaaataaacttctatcttgtttaagccactgttattttgggttttctgttactcgcagccgaacctaatcctaactgataca

SEQ ID NO: 88 - DNA, 1387 bp, Homo sapiens
  source 1..1387: mol_type = genomic DNA
gaaattggtacctagaagtggggtgctgccataacaaaaacctaaaatatgtggcattggcttagcggtcgggcggcgggcggcgaggaaacngatatcgnaggctggaaagntggngacccntgttatgcagtggcaaaacatttggtaaaactgtcgcctgcgataacttggaaggcagaccacgtgcctactgagcctgtagctctaggggaagnggttggaaaaantcagaatgttagtgtgtgttggctgctnctngctgcntttagcaaggtattacaagaaagagatgagctcaggaaagaattggccggtttgcaagcagaaatgaaagggaatagagagagtccagaaatttggggccttgcagggttggaaaagccaactgcttctgnaccccaaacagtaagagataagactgaaaaaggctttgagcaacaaaggcccattaagacttctcgccagacaaagggactcagccctgcggcaaagatcagattaagggtgttgccttcccacccaagcctattgtttcagatggcctcaaggtagccgccattaagttgagagagaggggatgggcagagcacagaggccagnaaataaaagantaaagcaggcttgagaactatgtctaggaaagaactttggntgtggttactggcacatggaactgactggaagcaaatagatcagaagcctactaagtttttgagggaattgtattgccaaagaaaccacaagcctggcctgnaaaagcctgtgactgttcganccctaaaacaacccttgggcccccaaacttgcaccagcaggaagcgggctgcgaaagctgtgcagcccccaaggagggcatactccccaacgcccacttcagatgtggccanggaggataatggacaaggaagaacctcccagagggcggagccaggggccacggagaacaatggacaagggagttcctcccagagagcagaatcagggtctaatcaaggaacttcccccactgccagggcagggggtcttcacaatncctgcccagcaggatttcanaattgctatggaccagtgactgctgtgtgtctcccattcttcccttttccgaatgggagtttttattgcggttatcctgtccctgctccaccattgtatattgggtgtgtggggggcagataacttgtcttttagttcataggtcgccggaccacgaggagccacatccggacctgatggagaggactgcgcatcacccagagatcctggactttgagctggatgcagtaactggatgggactttgggttgtctcccttggggagggggtgagtgtgttctatgtgtgggaagaagggtgnaacggatatttggtggccagaggggcagac

SEQ ID NO: 89 - DNA, 444 bp, Homo sapiens
  source 1..444: mol_type = genomic DNA
tgtggcagagactggctagctgttcaccaaacccgtttcctcttcttcctgggcacacagctagactacatttcccagcctcccttgcagttaggtgtggccatgtgactgagttctggccaatggaatgtgagcagaagtgatgtgtgccacttccaggcctggcccataaaaacctcccatgcatnctcctctctattctctcttccctctctgctggctggatgtcgacgcccagggcgaccttggaagccacgtgttgaagatggcagagcctccgtcagcctgggtccctgaatgactgcgtggagcagagccccccccnacccccccagcacatccaattggactttacgtgagcgagaaataaacttctattgtgttaagccactgagattttggggtttatctgttacagcagctagcgttaccttaactaataca

SEQ ID NO: 90 - DNA, 448 bp, Homo sapiens
  source 1..448: mol_type = genomic DNA
tgtggcagagactggctagntgttcaccaaancccgtttccttttcctcctgggcacacagctagactacatttcccagcctcccttgcagttaggtgnggccatgtgactgagttctggccaatggaatgtgggcggaagtgatgtgcgccacttccaggcctggcccataaaaacctcccacgcgatcctccacgctctctctctttccccatctgccggctggatgcagaggatccagtggaggactccgaggccctaggggatggcggagccacaagatggaaggagcctgggtccctgaatgaccgcgtggagcagagccgcccaccccccagaaccacccgcattggactgtgacgtgagcgagaaataaacttttattgtgttaagccactgagatttgggggttgtttgttacagcagttagcctaccctgactaataca

SEQ ID NO: 91 - DNA, 595 bp, Homo sapiens
  source 1..595: mol_type = genomic DNA
tgtagtggacatctgttgtttttgcctgcccagcatccattcccccttcttctggtaacagcaccccgattttcctttggggaaccacccctcccccactctcagtccatgtggttcgggtggggctgaccccacccctcggctccaggggtgggcacgtgacccaggcctggccaatcagagcattccatccccctggccacagtgattggttcagggatgggcacgtgacccaagccgggccaatgagagtcagccctgggacttttgctggaactattgggaaagagangctctctttccgctggggttgctaagctggnaggatgtaagcctggagctgctggcggccatcttgccaccacgtggggagagcctgcctgagaatgaagccaacacagaggaaagcagagccgagagatggagagagacagantcctgatgacatcgtttgagcncctggatccagccgtgcctgaagccaganctacccctggacttttcagttacgtgagccaataaattcccttttttgcttaagccagtttgagttgggtttctgtcacttgcaaccgaaagagtcctgactaataca

SEQ ID NO: 92 - DNA, 610 bp, Homo sapiens
  source 1..610: mol_type = genomic DNA
tgtggcagacactgttggttgcctacccaacagccattctccccttcttccttgctaacagaaccccgattttgttcaggtatcnggcggccatgtgcttcaggggaggctggncccctccccagccccagggggtgaatcntgattggtctaagccaatcatggtaatcccattccccttgccagtgattggtttaggaatgggcatgtgacncaattctggccaatgagacgtgaggggaagtctgctggggggcttctgggaaagntttcctcgctcttaaaaaaaggacataaggaagagatgtcctcttttcttgcctctggacgttgtcgtgtgnggatgtgatgcctggagctgcngcagccatcttgcgaccatgaggggacaagcctgagaggaaaagccaacacgctgaggatggcagagcggaaagatggaaagaacctgggtccttgatgacatcgttgagccgctgaattaaccaaccctggaaccgccctacctccggacttcttgttatgtgagataataaattttccttattgtttaagccantttgagttgggttttctgttacttgcagccgaaagcatcctaactgataca

SEQ ID NO: 93 - DNA, 672 bp, Homo sapiens
  source 1..672: mol_type = genomic DNA
tgtactagacatgtcaatcacggtgcctgccacgcccacagccccttctaagggaaactgccccgcccacagcccctgctgaaggggcagccctaggcggccatgttttgtaccacgtgaccctgccctcccctggccacagctgattggaccaggggtgggcacctgacccaagggcagccaatccataggctggccagcgacctatgacgtggcctggcgcgaaaagatgagctgggccaatcagattctctctctcgggaatttgaactgggaaacacggagagaatgaggcagttagcagcgggagctgaagctgaaaggatgcatagagagaagccatgaggtagagtcggggccatgatgggccatgtgcaagccgaagttatgaggaagcagaaactatgagtaagcagaggaagccggtcggtagagagaagagaacggagcagacgcgcagagagaagcngagacgcgtgatgagagagggaccggacgagagcngcngaggtccctagagctgccccggttccggccgcttccagtccctgttcctggcgntttcccagttccagttccggtccacgtgtatccttacaataaaccccccttttcttgagntaacttgagtgagtctctgttccttgcaaccaaaagagcctaactaaaaca

SEQ ID NO: 94 - DNA, 562 bp, Homo sapiens
  source 1..562: mol_type = genomic DNA
tgtagtggacatttgtcattctttttggccgcccagcatctgaacncccttcctatgtttggggaattccccaccttatgagtcccgcctccccaaggtagaagccagaaactcgctttcccagcctcccttgcagctagggcgcgggcacgtgacctaggctccgccaatcagacgcacccgcnccagactttgaatcggaagctagtgacgcaaggaagcagggaccgcgcggaatccattctctggcgagggtggcggcagctggcatccagtttccagaggcagcagtggcagnggttctagcggcggcgtccagcgctcatgtgtggtgcaagctgcggtgtctgtgcccagcggcggcagcagtggtgtcctcaccggaccagttctgcggcgtgattttgggcattgttcctggctgcgtagcctccaagcctggttctccggccctcccggagattctgtgagctacccaatatcctttcaataaattccttttctgcttaaattagccagagtcggtttctgttgcttgcaactaagaaccctgactgataca

SEQ ID NO: 95 - DNA, 542 bp, Homo sapiens
  source 1..542: mol_type = genomic DNA
tgtagtggacaccttttggggtgtctgcccagccccccttctctgggaaccgcccccccacccacaggggtgggcccccggcagccatgtttgtactacgtgaccccgcccccctggccacagctgattggaccaggggtggacacctgacccaagctgggccaatcagattctctctcccgggaatttggaattgggactnagagatnctagtcagtctctgctggtcgcttgaactgggganatgtaaactcgggagctgtggggcggccatnttccgccatgtgnacggagaagcagagaaagccggtctgcagagagaggagaatgaagcagacgcgcagagagaagcagagacgagagaccgggtggcctcagagacagagaaagatggagccgattctctcctgaggcccggctgcattcctgcccttgggttccgtgagacacccctgtatccttataataaattccccttttttgcttaagctagctcgagtgggtttctgttacttgcaaccaaaagagccttgactaagaca

SEQ ID NO: 96 - DNA, 444 bp, Homo sapiens
  source 1..444: mol_type = genomic DNA
tgtgatggttaatactgagtgtcaacttgattggattgaaggatacaaagtattgatcctgggtgtgtctgtgagggtgttgccaaaggagattaacatttgagtcagtgggctgggaaaggcagacccacccttaatctgggtgggcacnatctaatcagctgccagcgcggctagaatataagcaggcagaaaaacgtgaaaagagagactggcctagcctcccagcctacatctttctcccgtgctggatgcttcctgccctcgaacatcggactccaagttcttcagttttgggactcggactggctctccttgctcctcagcttgcagacggcctattgtgggaccttgtgatcgtgtgagttaatacttaataaactcccctttatatatatatntattccattagttctgtccctctagagaaccctgactaataca

SEQ ID NO: 97 - DNA, 453 bp, Homo sapiens
  source 1..453: mol_type = genomic DNA
tgtgatggttaatactgagtgtcaacttgattggattgaaggatgcaaagtattgttcctgggtgtgtctgtgagggtgttgccaaaggagattaacatttgagtcagtggactgggagaggcagacccaccctcaatctgggtgggcaccatctaatcagctgccagcgcggccagaatanaaagcaggcagaagaacgtggaaagactagactggctnagtcttccggcctncatctttctcccgtgctggatgcttcctgccctcgaacatcggactccaagttcttcagctttgggactcggactggcttccttgctcctcagcttgcagacggcctattgtgggacctcaccttgtgatcgtgtgagtcaatactccttaataaactcccctttatatatacatctatcctattagttctgtccctctagagaaccctgactaataca

SEQ ID NO: 98 - DNA, 520 bp, Homo sapiens
  source 1..520: mol_type = genomic DNA
tgtgatggttaattttatgtgtcaacttgactgggccangggatgcccagatagctggtnaaacattatttctgggtgtgtctgtgagggtgtttccggaagagattagcatttgaatcggtagactgagtaaagnagatcgccctccccaatgtgggtgggcatcatccaatccgttgagggcctgaatagaacaaaaaggcggaggaagggngaattcgctctctctgcttgagctgggacatccgtcttctcctgccctcggacatcggcgctcctggttctcgggccttcggactcggactgggacttacaccatcggctcccctggttctcaggccttcggactcggactggaactacaccaccggctttcctgggtctccagcttgcagacggcagatcgtgggacttctcagcctccataatcgcgtgagccaattcctcataataaatctctttctatatatctatatatatcctattggttctgtttctctggagaaccctgactaataca

SEQ ID NO: 99 - DNA, 515 bp, Homo sapiens
  source 1..515: mol_type = genomic DNA
tgtgatggttaattttatgtgtcaacttgactgggctaagggatgcccagatagctggtaaaacattatttctgggtgtgtctgtgagggtgtttccggaagagattagcatttgaatcagtagactgagtaaagaagatccgccctcaccaatgtgggcgggcatcatccaatccgttgagggcccgaatagaacaaaaaggcggaggaagggcgaattcnctctctcttcttgagctgggacatccatcttctcctgccctcggacatcggagctcctggttctcgggccttcggactccgggacttacaccagcggcccccctggttctcaggccttcggactcggactgaattacaccaccggctttcctggttctccagcttgcagacggcanatcgtgggacttctcggcctccataatcgcgtgagccaattcccataataaatctcctcttatatatctctatatatcctattggttctgtttctctggagaaccctgactaataca

SEQ ID NO: 100 - DNA, 554 bp, Homo sapiens
  source 1..554: mol_type = genomic DNA
tgtgatggttaatattaggtgtcaacttgattggattgaaggatgccnagatagctggtaaagtattgtttctgggtgtgtctgtgagggtgttgccagaggagattaacatttgagtcagtggactgggagaggaagacccaccctcancgatgtgggtgggcaccatccaatcggctgccggcncggctagaacaaaaaggcagaggaagggcgaatttgctctctctcctggagctgggacacccttctcctcctgccctcggacatcaganctccaggttcttcggcctttggactctnggacttacaccagcggtttnccgggctctcgggccttcggccacagactgaaggctgcaccgtcggcttccctgcttttgaggctttnggactcggactgagccacgctaccggcttccctggctccccagcttgcagacggcctatcgtgggacttcncggccttcgtgatcgtgtgagccaattcccctaataaatcccctttatctatcctattagttctgtccctctggagaaccctgactaataca

SEQ ID NO: 101 - DNA, 556 bp, Homo sapiens
  source 1..556: mol_type = genomic DNA
tgtgatggttaattttatgtgtcaacttgactgggccacggggtgcccagatatttggtcaaacattattctgggtgtgtctgtgagggtgtttctggnatgagattaacatttgaatcggtagactgagtaaagcagattgccctccctaatgtgggtgggcctcatccaatcagttgaaggcctgaatagaacaaaaaggctgaccctcccccgagtaagagggaattcctcctgcctgactgccttcgagctgggacatcggtctttttcctgccttcggactcgaactgaaacatcggctcttcctgggtctcgagcctgccggccttcggactggaactacaccatcggctctcctggttctcaggccttcggactcggactggaactacaccatcggctctcctgggtctccagcttgccgactcgnnntgcagatcttgggacttntcagcctccataatcgcgtgagccaattccttataataaatctctctcttntatatatataccacatcctattggttctgtttctctggagaaccctaataca

SEQ ID NO: 102 - DNA, 717 bp, Homo sapiens
  source 1..717: mol_type = genomic DNA
tgtgatggttgattttgggtgtcaacttgactggattaagggatacccagatagctggtaaagcattatttattctcaatcancgcatnaattattctcaatgcttcagtaggcactgagcccgtccctcttctgctgaaagggaaacccaggtggtttggcatttgattagaatgattgggctgccccaggtgtgtctgtgagggtgtttccggaggagattggcatgtgagtcggtggactgagtggggaagatccgccctcaatgtgggcgggcaccatccaatcggctgggggcccggatggaacaaaaaggcagaggaagggcgaattcttgcnctctctcttccggagccgggacgcccttcttctcctgcccttggacgtcagaactccaggttctccggcctttggactccgggacttgcaccagcggcccccgggctctcaggccttcggcctcggactgagnnactgngagttacaccatcggcttccctggttctnaggccttcggacttggactgagccacgccaccggcttccctggntctccagcttgcagatgcagncggcctatcgtgggacttctcagcctccgtaatcgcgtgagccaattcccctaataaatcccttctcatatatctntctatatatatatatatntntntatatatcctattggttctgtctctctggagaaccctgactaataca

SEQ ID NO: 103 - DNA, 397 bp, Homo sapiens
  source 1..397: mol_type = genomic DNA
tgtgatggttagttttatgtgtcaacttggctaggctatagtacccagttatttaatcaaacactaatctaggtgttgctgtgaaggtattttgtagatgtggttaacatctacaatcagttgactttaagtaaaggagattaccctcgataatgtgggtgggcctcatccaatcagttgaaggccttaagagcaaaaactgaggtttcccggagaagaagaaattctgcctcaagactgcagcatcaactcctgcctgagtttccagcctgccggcctgccctacggatttcggacttgccagcccccacaatcgcgtgagccaattccttaaaataaatctctttctacacacatcctattggttctgtttctctggagaaccctgactgataca

SEQ ID NO: 104 - DNA, 464 bp, Homo sapiens
  source 1..464: mol_type = genomic DNA
tgtgatggttaattttatgtgtcaacttggctaggctatggtgcccagttgtttggtcaaacactagtctagatgttgctgtgaaggtattttttagatgtgattaacatttacaatcagtngactttgagtaaagcagattaccctccataatgtgggtgggcctcatccaatcagttgaaggccttaagagcaaagactgaggtttcccgaagaagaaggaattctgcctcaagactgcaacatagaaatcctgcctgagtttccagcctgctggcctgccctgcggatttcggactcaagactgcaacatcaactcttacctgaatttccagcctgccggcctgccctacagatttcggacttgccagcccccacaatcgcgtgagccaattccttaaaataaatctctctctacacatatcctattggttctgtttctctggagaaccctgactaataca

SEQ ID NO: 105 - DNA, 414 bp, Homo sapiens
  source 1..414: mol_type = genomic DNA
tgtgatggttaattttatgtgtcaacttggctaggccacggtgcccagatatttggtcaaacattantctggatgtttctgtgaaggtattttttagatgagattaacatttaaatcagtagactttgagtaaagcagattaccctccataatgtgggtgggcctcatccaatcagttgaaggccttaagagaaaaagactganntcccccgaggaagagggaattctgccnncagactgccttcggactcgagctgcaacatcaactcttccctgggtctccagcctgccggcctaccctgcagattttggacttgccagcctccacaatcgcgtgagccaattccttaaaataaatctctctctacacacatcctattggttctgtttctctggagaaccctgactaataca

SEQ ID NO: 106 - DNA, 628 bp, Homo sapiens
  source 1..628: mol_type = genomic DNA
tgtgctggattgtnctgttgtcaacttggttaagctgggaactacgtttcccagaatccccttccctgtatggttctgggttagagttggccaaagaggaacttgcgcgagatttgggaggcggaagtgaagcagcagccattactctctgaaggtcgtcgcggtcagacgcggtgagagacagacgcagaggtgcccagcgggttccagcttgtcctcgctctcccccgctccgcgtccagctcttcttcccgactgccggccctgctgaccaacagcggccccaggcccaccaccagacgcttggctgcggacccacagaggcggtagccacgcagaggcaacagctttccatagacctctccacnagctccccttcatggtcccacttcagcggctggacgtgcctggcttctcagatttccccgcaagctccgactcgtccacccgcgccagtgcttcaggaggggctggttagtgactttctctgatcctccaactcccccttccagaccttcacttccccagctcctcccacaattgtgtaaggtctaattcctataataaatcccttatcccataacactcatagtggctctgcttccctgactgaaccctgactgataca

SEQ ID NO: 107 - DNA, 663 bp, Homo sapiens
  source 1..663: mol_type = genomic DNA
tgtggtggctttgtaatgtgtcaacttggctaggctgaactacgtttcccagaattcccttccctgtatgtttccggttagggtgggccacaagaganattctgcgcgagatttggagggcggaagtgaagcagcagccatnttgtttttnatgctcggaaggtcggngcagggnccgcaggcgctnttgcagctcacgcacgttgtcgctnatctgctggctcacctcgttggcgtggggcagcagccgggcctgcagctgctccaccttcccctggatcctccttcagcttctccgactcctgggccaggtgtgtgtttagctccgtgacgaagggcgccagcttctcctgcaggacacccgcatcatcaaggtcggaggcagcgagagactgacacgggttccagtccgtcctcgtgggttccagctcgtgctcgtgggttccagcttgtccttgctctcccccacttcacatccatcttcccttcccgactgcctgccctgcggacttcaagctccagcatcagacgcaaagacaacagccttacagagactgcttaaccagctcccacaattgcgtaaggtcaaatccctgtaacaaatcccttattatatatatctcctagtggttctgcttctctgattgaaccctgactgataca

SEQ ID NO: 108 - DNA, 1651 bp, Homo sapiens
  source 1..1651: mol_type = genomic DNA
gaaaattggtaccgaggagtggggcattgctataaagatacctgaaaatgtggaagcggctttggaactgggtaacgggcagaggttggaagagtttggagggctcagaagaagacaggaagatgagggaaagtttggaacttcttagagactggttaaatggttgtgaccaaaatgctgatagtgatatggacagtgaagnccaggctgacgaggtctcagatggaaatgaggaacttattgggaactggagcaaaggtcacccntgttatgccttagcaaagaacttggctgcattgtgtccatgccctagggatctgtggaagtttgaacttaagagtgatgacttagggtatctggcggaagaaatttctaagcagcaaagcgttcaagatgtggcctggctgcttctaacagcctacgntcagatgcgggagcaaagaaatgacttaaagttggaacttatatttaaaagggaagcagagcgtaaaagtttggaaaatttgcagcctggccatgtggcagaaaagaaaaanccattttcaggagaggaattcaagcaggctgcggagcaaccacttgctagagagatttgcatgactaaaagggagccaagtgctaatanccaagacaatgggaaaaaggcctcgaaggcatttcagagatcttcgaggcagcccctcccatcacaggcccagaggcctaggaggaaagaatggtttcgtgggccaggcccagggccccgctgccctgcgcagcctcgggacactgctccccgcatcccggccgctccggctccagccgcggctcaaagggccccaggtacagctcgggccgccgcttcggagggcgcaagccataagccttggcggcttccacgtggtgttaagcctgcaggcgcacagaatgcaagagtgaaggaggcttggcagcctccgcctagatttcagaggatgtatgggaaagcctgggtgcccaggcagaagcctgctgcaggggcggagccctcacagagaacctctactagggcagtgccgaggggaaatgtggggttggagcccccacacagagtccccaccggggcactgcctagtggagctgtgggaagggggccaccgccctccagaccccagaatggtaganccaccggcagcttgcaccctgagcctggaaaagccgcaggcactcaactccaacccgtgagagcagccacgggggctgnaccctgcaaagccacaggggcggagctgcccaaggccttgggagcccaccccttgcaccagtgtgccctggatgcgggacatggagtcaaaggagattattttggagctttaagatttaatgactgccctgctgggtttcggacttgcgtggggcctgtagcccctttcttttggccgatttctcccttttggaatgggaatgtttacccaatgcctgtaccnccattgtatcttggaagtaaataacttgtttttgattttacaggctcataggtggaaggaacttgccttgtctcagatgagactttggactttggacttttgagttaatgctggaatgagttaagactttgggggactattgggaaggcatgattgtattttgcaatgtgagaaggacatgagatttgggggggccaggggcggaa

SEQ ID NO: 109 - DNA, 428 bp, Homo sapiens
  source 1..428: mol_type = genomic DNA
tgatatggtttggatctgtgtccccgcccaaatctcatgtcgaattgtaatccccagtgttggaggnggggcctggtgggaggtgattggatcatgggggcggatttctcatgancggtttagcaccatccccttggtgctgttctcgtgatagtgagtgagttctcacgagatctggttgtttaaaagtgtgtggcacctcccccctcgctctctcttnctcctgctccggccatgtgacgtgcctgcttccccttcgccttccgccatgattgtaagtttcctgaggcctccccagaagccgannagatgccancgccatgcttcctgtacagcctgcggaaccgtgagccaattaaacctcttttctttataaattacccagtctcaggtatttctttatagcagtgcgagaacggactaataca

SEQ ID NO: 110 - DNA, 465 bp, Homo sapiens
  source 1..465: mol_type = genomic DNA
tgctatggtttggatgtttgtccccgccaaaactcatgttgaaatttgatccccaatgtggcagtgttggaggtggggcctagtgggaggtgtttgggtcatgggggcggatccctcatgaatagattaatgccctccctcgnggtgggngtgagtgagttctcgctctnncgcgggaatggattagttcccgcgagagcgggttgttaaaaagagtctggcgnctccctcctctctctctcttgcttgcttcctctctcgccatgtgatctctgcacacgcccgctccccttccncttcactttccgccatgagtngaagcagcctgaggccctcaccagatgcagctgcccgnaccntgctttttnnccagccaccagaatcgtgagccaaataaacctcttttctttataaattacccagcctcaggtattccgttatagcaacacaaaacggactaagaca

SEQ ID NO: 111 - DNA, 426 bp, Homo sapiens
  source 1..426: mol_type = genomic DNA
tgatatggtttggatgtttgtcccctccaaatctcatgttgaaatgtgatccccagtgttggaggtggggcctggtgggaggtgtttgggtcatgggggcggatccctcatgaatggcttggcgccgtccccntggtgatgagtgagttctcgctctgttagttcacgcgagatctggttgtttaaaagagtntggcacctcccccctctctctcttgctcccgctctcgccatgtgacgcgcctgctcccccttcgccttccgccatgattgnaagcttcctgaggcctcaccagaagccgagcagatgccggcgccatgcttcctgtacagcctgcagaaccgtgagccaantaaacctcttttctttataaattacccagcctcaggtattcctttatagcaacgcaagaacggactaacaca

SEQ ID NO: 112 - DNA, 432 bp, Homo sapiens
  source 1..432: mol_type = genomic DNA
tgctatagtttggatgtttgtcccctccaaacctcatgttgaaatttgatccccagtgttggaggtggggcctaatgggaggtgtttgggtcatgggggcggatccctcatgaatggnttggtgccctccctcggggtggggatgagtgagttctcgctctattagttcccgcgagagctggttgttaaaaagagcctggcacctccctcctctctctcttgcttcctctctcgccatgtgatctctgcacacgccggctccccttcgccttccgccatgagtggaagcagcctgaggccctcaccagaagcagatgctggcgccatgcttcttgtacagcctgcagaaccgtgagccaaataaacctcttttctttataaattacccagcctcaggtattcctttatagcaacacaaaacggactaagaca

SEQ ID NO: 113 - DNA, 457 bp, Homo sapiens
  source 1..457: mol_type = genomic DNA
tgctatggtttggatatggtttgtttgtccccaccaaaactcatgttgaaatttgatccccaatgtggcggtgttgggaggtggggcctagtgggaggtgtttgggtcatgggggcggatccctcatgaatggcttggtgccgttctcgcggtagtgagtgagtgagttctcgctctcgcgagactggattagttctcgcgggaatggattagttcccgcgagagtgggttgttataaagccaggacgcccctcgggttttgcctcttcgcacgtgtccgcttcccctttgaccttctccgccatgttntgacgcagcacgaaagccctcaccagaagccagggccatgcccttgaacttcccagcctgcagaaccgtgagctaaataaacctcttttctttataaattacccagtctcaggtattctgttatagcaacacaaaacggactaagaca

SEQ ID NO: 114 - DNA, 428 bp, Homo sapiens
  source 1..428: mol_type = genomic DNA
tgctatggtttgaatgtttgtcccctccaaaactcatgttgaaacttaatccccaatgtggcagtattgagaggtggggcctttaagaggtgattgggtcatgagggctctgccctcatgaatggattaatccattcatggattaatggattaatggattaatgggttatcatgggagtgggactggtggctttataagaagaggaagagagacctgagctagcacgctcagccccctcgccatgtgatgccctgcgccgcctcgggactctgcagagagtccccaccagcaagaaggccctcaccagatgcggcccctcgaccttggacttctcagcctccanaactgtaagaaataaattccttttctttataaattacccagtttcaggtattctgttataagcaacagaaaacggactaagaca

SEQ ID NO: 115 - DNA, 396 bp, Homo sapiens
  source 1..396: mol_type = genomic DNA
tgctatggtttgaatgtgtcccccaaagttcatgtgttggaaacttaatccccaatgcaacagtgttgggaggtggggcctnntaagaggtgattaggtcatgagggctccgccctcatgaatggattaatgccgttatcgcgggagtgggttagttatcgcgggagtgggttcgttataaaagnaagttcggcccccttttgctctctctctctcgctctcttgcccttccgccttccgccatgggatgacgcagcaagaaggccctcgccagatgccggcncctcgatcttggacttcccagcctccagaaccgtgagccaaataaatttctgttctttataaattacccagtctgtggtattctgttatagcagcacaaaacggactaagaca

SEQ ID NO: 116 - DNA, 1580 bp, Homo sapiens; identical to SEQ ID NO: 9
  source 1..1580: mol_type = genomic DNA

SEQ ID NO: 117 - DNA, 355 bp, Homo sapiens
  source 1..355: mol_type = genomic DNA
tgatatggtttggctgtgtccccacccaaatctcaacttgaattgtatctcccagaattcccacgtgttgtgggagggacccagggggaggtaattgaatcatgggggccggtctttcccgtgctattctcgtgatagtgaataagtctcacgagatctgatgggtttatcaggggtttccgcttttgcttcttcctcattttcctcttgccgccgccatgtaagaagtgcctttcgcctcccgccatgattctgaggcctccccagccatgtggaactgtaagtccaattaaacctctttttcttcccagtctcgggtatgtctttatcagcagcgtgaaaacggactaataca

SEQ ID NO: 118 - DNA, 364 bp, Homo sapiens; identical to SEQ ID NO: 8
  source 1..364: mol_type = genomic DNA

SEQ ID NO: 119 - DNA, 375 bp, Homo sapiens
  source 1..375: mol_type = genomic DNA
tgatatggtttggctgtgtccccacccaaatctcatcttgaattgtagttcccataatccccacgtgtcgtgggagggacccggtgggaggtaattgaatcatgggggcggttacccccatgctgttctcgtgatagtgagtgagttctcacgagatctgatggttttataaggggcttttcccccttcgctcggcacttctccttcctgccgccatgtgaagaaggacgtgtttgcttccccttccgccatgattgtaagtttcctgaggcctccccagccatgcngaactgtgagtcaattaaacctctttcctttataaattacccagtctcgggtatgtctttattagcagcgtgagaacggactaataca

SEQ ID NO: 120 - DNA, 381 bp, Homo sapiens
  source 1..381: mol_type = genomic DNA
tgatatggtttggctgtgtccccacccaaantctcatctcgaattgtaatccccataatccccacgtgtcgagggagggacctggtggaggtgattggatcatgggggcggtttcccccatgctgttctcgtgatagtgagtgagttctcacgagatctgatggttttataagtgtctggcagntttcccctgcnctcacncttctctctcctgccgccntgtgaagaaggtgcttgcttccccttcgccttccgccatgattgtaagtttcctgaggcctccccagccatgcggaactgtgagtcaattaaacctctttcctttataaattacccagtctcgggtagttctttatagcagtgtgaaaacggactaataca

SEQ ID NO: 121 - DNA, 1631 bp, Homo sapiens
  source 1..1631: mol_type = genomic DNA
gaaaattggtaccgggagagtggggcactgctataaagatacctgaaaatgtggaagcgactttggaactgggtaacgggcagaggttggaacagtttggagggctcagaagaagacaggaagatgtgggaaagtttggaacttcctagagacttgttgaatggttttgaccaaaatgctgatagtgatatggacaatgaagtccaggctgaggtggtctcagatggagatgaggaacttattgggaactggagtaaaggtcactcttgctatgctttagcaaagagactggcggcattttgcccctgccctagagatctgtggaactttgaacttgagagagatgatttagggtatctggcggaagaaatttctaagcagcaaagcattcaagatgtgacctggctgcttctaaaagcgtacggtcatatgcgttcacaaagagatggtctgaaattggaacttatgtttaaaagggaagcagagcataaaagtttggaaaatttgcagcctgaccatgcggtagaaaagaaaaacccattttctggggagaaattcaagccggctgcagaaatttgcataagtaacgaggagccgaatgttaatagccaagacaatggggaaaatgtctccagggcatntcagagaccttcgcggcagcccctcccatcacaggcccggaggcctaggagggaaaaatggtttcgtgggccgggcccagggccccgctgctctgtgcagcctcgggacatggcgccctgcgtcccagccgctccagctccagccgtggctaaaaggggccaaggtacagctcgggccgttgcttcagagggtgcaagccccaagccttggcggcttccacgtggtgttgggcctgcgggtgcgcagaagncaagagttgaggtttgggaacctccgcctagatttcagaggatgtatggaaacgcctggatgtccaggcagaagtctgctgcaggggcggagccctcatggagaacctctgctagggcagtgcggaggggaaatgtggggttggagcccccacacagagtccccactggggcactgcctagtggagctgtgagaagagggccaccgtcctccagaccccagaatggtagatccaccgacagcttgcaccgtgcgcctggaaaagccgcaggcactcaacgccagcccgtgaaagcagccgcgggggctgtaccctgcagagccacaggggcggagctgcccaaggccntgggagcccaccccttgcatcagcgtgncctggatgtgagacatggagtcaaaggagattattttggagctttaagatttaatgactgccctgctgggtttcggacttgcatggggcctgtagcccctttgttttggccaatttctcccatttggaatgggagcatttacccaatgcctgtacccccattgtatcttggaagtaactaacttgcttttgattttacaggctcataggcggaagggacttgccttgtctcagatgagactttggacttggacttttgagttaatgctggaatgagttaagactttgggggactgttgggaaggcatgattgtgttttgaaatgtgagaaggacatgagatttgggaggggccaggggcggaa